(19) United States
(12) Patent Application Publication
(10) Pub. No.: US 2024/0270740 A1
(43) Pub. Date: Aug. 15, 2024

(54) INHIBITORS OF CYCLIC GMP-AMP SYNTHASE AND USES THEREOF

(71) Applicant: Ventus Therapeutics U.S., Inc., Waltham, MA (US)

(72) Inventors: Ramsay Beveridge, Pointe-Claire (CA); Jason Burch, Pointe-Claire (CA); Stephane Ciblat, Montreal (CA); Patrick Cyr, Saint-Constant (CA)

(21) Appl. No.: 18/544,945

(22) Filed: Dec. 19, 2023

Related U.S. Application Data
(60) Provisional application No. 63/501,320, filed on May 10, 2023.
(60) Provisional application No. 63/433,987, filed on Dec. 20, 2022.

(51) Int. Cl. C07D 417/14; A61K 31/433

(52) U.S. Cl. CPC C07D 417/14; A61K 31/433

(57) ABSTRACT

The present disclosure relates to compounds of Formula (I):

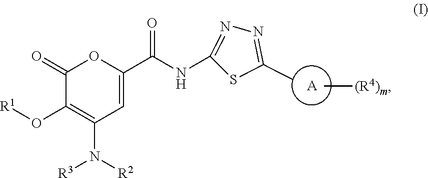

or a pharmaceutically acceptable salt thereof, wherein Ring A, R¹, R², R³, R⁴, and m are described herein, methods of preparation, methods of treatment, and pharmaceutical compositions comprising same. The present disclosure further relates to the use of the compounds of Formula (I), and pharmaceutically acceptable salts thereof, in the treatment of cGAS-related diseases and disorders.

### RELATED APPLICATIONS

This application claims priority under 35 U.S.C. § 119(e) to U.S. provisional patent applications. U.S. Ser. No. 63/433,987, filed Dec. 20, 2022, and U.S. Ser. No. 63/501,320, filed May 10, 2023, the entire contents of each of which are incorporated herein by reference.

### BACKGROUND

Aberrant accumulation of cytosolic DNA induces type I interferons and other cytokines that are important for antimicrobial defense but can also induce autoimmunity. This DNA signaling pathway requires the stimulator of interferon genes (STING) adapter protein and the transcription factors NF-κB and IRF3, but the mechanism of DNA sensing was unclear until recently. It is now understood that mammalian cytosolic extracts synthesize cyclic GMP-AMP (cGAMP) in vitro from ATP and GTP in the presence of DNA rather than RNA (WO 2014099824). DNA transfection or DNA virus infection of mammalian cells also trigger the production of cGAMP. cGAMP binds to STING, leading to IRF3 activation and induction of interferon-β (IFNβ). Thus, cGAMP is the first cyclic dinucleotide in metazoans, and cGAMP functions as an endogenous secondary messenger that induces interferon production in response to cytosolic DNA.

cGAMP synthase (cGAS) is an enzyme that intervenes in the synthesis of cyclic GMP-AMP and belongs to the nucleotidyltransferase family. Overexpression of cGAS activates the transcription factor IRF3 and induces IFNβ in a STING-dependent manner. Knockdown of cGAS inhibits IRF3 activation and IFNβ induction by DNA transfection or DNA virus infection. cGAS binds to DNA in the cytoplasm and catalyzes cGAMP synthesis. These findings indicate that cGAS is a cytosolic DNA sensor that induces interferons by producing the second messenger cGAMP.

The critical role of cGAS in cytosolic DNA sensing has been established in different pathogenic bacteria, viruses, and retroviruses (US 20210155625). Additionally, cGAS is essential in various other biological processes, such as cellular senescence and recognition of ruptured micronuclei in the surveillance of potential cancer cells.

There is a need for therapeutic agents that target cGAS. Small molecule inhibitors that are specific for cGAS would be of great value in treating diseases that arise from inappropriate cGAS activity and the resulting undesired type I interferon activity. This present disclosure is intended to fill this unmet need associated with current cGAS inhibition therapy.

### SUMMARY

Provided herein are cGAS inhibitors of Formula (I):

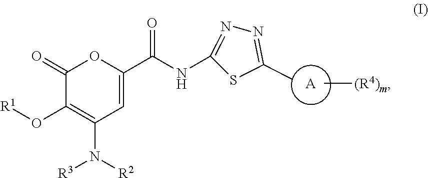

or pharmaceutically acceptable salts thereof, wherein Ring A, R¹, R², R³, R⁴, and m are described herein.

Further provided are methods of preparation, methods of treatment, and pharmaceutical compositions comprising same.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistoy, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, IN 1972). Compounds described herein can additionally encompasses individual isomers substantially free of other isomers, and alternatively, as mixtures of various isomers.

Unless otherwise stated, compounds described herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In some embodiments, the alkyl group is an unsubstituted C₁₋₁₀ alkyl (e.g., —CH₃). In some embodiments, the alkyl group is a substituted C₁₋₁₀ alkyl.

“Haloalkyl” refers to a substituted alkyl group, as defined herein, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 10 carbon atoms and one or more carbon-carbon double bonds (e.g., 1, 2, 3, or 4 double bonds) (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In some embodiments, the alkenyl group is an unsubstituted C₂₋₁₀ alkenyl. In some embodiments, the alkenyl group is a substituted C₂₋₁₀ alkenyl.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₉ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₃), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In some embodiments, the alkynyl group is an unsubstituted C₂₋₁₀ alkynyl. In some embodiments, the alkynyl group is a substituted C₂₋₁₀ alkynyl.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 9 ring carbon atoms (“C₃₋₉ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₇ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in some embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons designate the number of carbons in the polycyclic ring system. Unless otherwise specified, each instance of a carbocyclyl group is independently unsubstituted (an “unsubstituted carbocyclyl”) or substituted (a “substituted carbocyclyl”) with one or more substituents. In some embodiments, the carbocyclyl group is an unsubstituted C₃₋₁₄ carbocyclyl. In some embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In some embodiments, the cycloalkyl group is an unsubstituted C₃₋₁₄ cycloalkyl. In some embodiments, the cycloalkyl group is a substituted C₃₋₁₄ cycloalkyl.

“Heterocyclyl” or “heterocyclic” refers to a radical of a 3- to 14-membered non-aromatic ring system having ring carbon atoms and 1 to 4 ring heteroatoms, wherein each ring heteroatom is independently selected from nitrogen, oxygen, and sulfur (“3-14 membered heterocyclyl”). It is understood that the ring sulfur or ring nitrogen may exist in an oxygenated state, such as an N-oxide (N—O), sulfonyl (S(═O)₂) or sulfinyl (S═O) ring heteroatom. In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes (i) polycyclic ring systems wherein the heterocyclyl ring, as defined above, is fused (e.g., spiro-fused or ring fused) or bridged with one or more carbocyclyl groups wherein the point of attachment is either on the carbocyclyl or heterocyclyl ring, or (ii) polycyclic ring systems wherein the heterocyclyl ring, as defined above, is fused with one or more aryl or heteroaryl groups, wherein the point of attachment is on the heterocyclyl ring, and in such instances (i) and (ii), the number of ring members designate the number of ring members in the polycyclic ring system. Unless otherwise specified, each instance of heterocyclyl is independently unsubstituted (an “unsubstituted heterocyclyl”) or substituted (a “substituted heterocyclyl”) with one or more substituents. In some embodiments, the heterocyclyl group is an unsubstituted 3-14 membered heterocyclyl. In some embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetra-hydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydro-furo[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₋₄ aryl”; e.g., anthracyl). “Aryl” also includes polycyclic ring systems wherein the aryl ring, as defined above, is fused with one or more carbocyclyl or heterocyclyl groups wherein the radical or point of attachment is on the aryl ring, and in such instances, the number of carbon atoms designate the number of carbon atoms in the polycyclic ring system. Unless otherwise specified, each instance of an aryl group is independently unsubstituted (an “unsubstituted aryl”) or substituted (a “substituted aryl”) with one or more substituents. In some embodiments, the aryl group is an unsubstituted C₆₋₁₄ aryl. In some embodiments, the aryl group is a substituted C₆₋₁₄ aryl.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” also includes polycyclic ring systems wherein the heteroaryl ring, as defined above, (i) is fused with one or more carbocyclyl or heterocyclyl groups wherein the point of attachment is on the heteroaryl ring, or (ii) is fused with one or more aryl groups wherein the point of attachment is either on the aryl or heteroaryl ring, and in such instances (i) and (ii), the number of ring members designate the number of ring members in the fused polycyclic ring system. Polycyclic heteroaryl groups wherein one ring does not contain a ring heteroatom (e.g., indolyl, quinolinyl, carbazolyl, and the like), the point of attachment can be on either ring, i.e., either the ring bearing a ring heteroatom (e.g., 2-indolyl) or the ring that does not contain a ring heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In some embodiments, the heteroaryl group is an unsubstituted 5-14 membered heteroaryl. In some embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Halo” or “halogen” refers to fluorine (fluoro, —F), chlorine (chloro, —Cl), bromine (bromo, —Br), or iodine (iodo, —I) radicals.

“Partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties).

“Saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, haloalkylene is the divalent moiety of haloalkyl alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl. By way of example, alkylene may be a C₁₋₆ alkylene, which may be linear or branched. An alkylene may further be a C₁₋₄ alkylene. Exemplary C₁₋₄ alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Salt” refers to any and all salts.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Examples of pharmaceutically acceptable acid addition salts include, but are not limited to, salts formed from inorganic acids, such as hydrochloric acid, hydrobromic acid, phosphoric acid, sulfuric acid and perchloric acid salts, or salts formed from organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “free base” refers to a neutral non-ionized form of a compound which is not a salt or pharmaceutically acceptable salt.

A “leaving group” is an art-understood term referring to a molecular fragment that departs with a pair of electrons in heterolytic bond cleavage, wherein the molecular fragment is an anion or neutral molecule. See, for example, Smith, March Advanced Organic Chemistry 6th ed. (501-502). Exemplary leaving groups include, but are not limited to, halo (e.g., chloro, bromo, iodo) and sulfonyl substituted hydroxyl groups (e.g., —O-tosyl, —O-mesyl, and —O-besyl).

A “patient” or “subject” is used interchangeably herein, and refers to a mammal, e.g., a human, mouse, rat, guinea pig, dog, cat, horse, cow, pig, or non-human primate, such as a monkey, chimpanzee, baboon, or rhesus. In certain embodiments, the patient or subject is a human.

“Effective amount” or “therapeutically effective amount” are used interchangeably herein, and refer to an amount of the compound sufficient to provide a therapeutic benefit in the treatment of a disease, disorder or condition, or to delay or minimize one or more symptoms associated with the disease, disorder or condition in a subject in need thereof. An effective amount can encompass an amount that improves overall therapy, reduces or avoids symptoms or causes of disease or condition, or enhances the therapeutic efficacy of another therapeutic agent. The effective amount of a compound may vary depending on such factors as the desired biological endpoint, the pharmacokinetics of the compound, the disease being treated, the mode of administration, and the age, health, and condition of the subject.

“Disease”, “disorder”, “condition”, or “state” are used interchangeably herein.

“Treating” or “treat” or “treatment” describes the management and care of a subject in need thereof, for the purpose of combating a disease, condition, or disorder in the subject, and includes the administration of a compound, or a pharmaceutically acceptable salt thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition, and therefore includes: (1) delaying the appearance of clinical symptoms of the state, disorder or condition developing in a subject that may be afflicted with or predisposed to the state, disorder or condition but does not yet experience or display clinical or subclinical symptoms of the state, disorder or condition, (2) arresting, reducing or delaying the development of the disease or a relapse thereof (in case of maintenance treatment) or at least one clinical or subclinical symptom thereof, or (3) relieving or attenuating the disease. i.e., causing regression of the state, disorder or condition or at least one of its clinical or subclinical symptoms.

“Modulate”, “modulating” and the like, refer to the ability of a compound to change the activity of a particular biological process (e.g., cGAS activity) in a cell relative to vehicle.

“Inhibition”, “inhibiting”, “inhibit” and “inhibitor”, and the like, refer to the ability of a compound to reduce, slow, halt or prevent activity of a particular biological process (e.g., cGAS activity) in a cell relative to vehicle.

The phrase “at least one” refers to one instance or more than one instance.

The articles “a” and “an” are used in this disclosure to refer to one or more than one (i.e., to at least one) of the grammatical object of the article.

The term “and/or” is used in this disclosure to mean either “and” or “or” unless indicated otherwise.

### DETAILED DESCRIPTION

### i. Compounds

Provided herein are compounds of Formula (I):

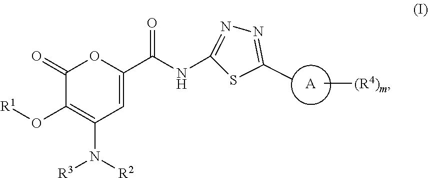

or pharmaceutically acceptable salts thereof, wherein:
Ring A is a 5-membered monocyclic heteroaryl;
R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄alkyl, or C₁-C₄ haloalkyl, wherein the alkyl and haloalkyl are independently substituted with 0, 1, 2, 3, or 4 R^{1D}; each R^{1C} is independently hydrogen, C₁-C₃alkyl, C₁-C₃ haloalkyl, or —OR^{1F}; each R^{1D} is independently halogen, —OR^{1F}, or —N(R^{1F})₂; and each R^{1F} is independently hydrogen, C₁-C₃alkyl, or C₁-C₃haloalkyl;
L₃ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}), or two instances of R^{1F} are taken together to form ═O;
R² is hydrogen or C₁-C₆ alkyl substituted with 0, 1, 2, 3, or 4 R^{2A}, and each R^{2A} is independently halogen, —OR^{2B}, or —N(R^{2B})₂, wherein each R^{2B} is independently hydrogen, C₁-C₃ alkyl, or C₁-C₃ haloalkyl;
or R¹ and R² are joined, with the atoms to which they are attached, to form a 6- or 7-membered heterocyclyl independently substituted with 0, 1, 2, 3, or 4 R^{1A};
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, —CN, C₁-C₃ alkyl, or C₁-C₃ haloalkyl;
each R^{3E} is independently hydrogen, C₁-C₄ alkyl, or C₁-C₄ haloalkyl;
each R⁴ is independently halogen, —CN, -L₂-OR^{4A}, -L₂-N(R^{4B})₂, C₁-C₆ alkyl, or C₁-C₆ haloalkyl, wherein each R^{4A} and R^{4B} are independently hydrogen, C₁-C₃ alkyl, C₁-C₃ haloalkyl, or —C(═O)R^{4C}, wherein R^{4C} is C₁-C₆ alkyl or C₁-C₆ haloalkyl;
each L₁ and L₂ is independently a bond, C₁-C₃ alkylene, or C₁-C₃ haloalkylene; and
m is 0, 1 or 2.

In some embodiments, provided herein are compounds of Formula (I):

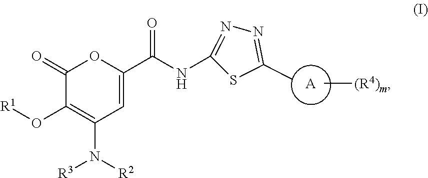

or pharmaceutically acceptable salts thereof, wherein:
Ring A is a 5-membered monocyclic heteroaryl;
R¹ is C₁-C₆ alkyl, C₂-C₆ alkenyl, or C₂-C₆ alkynyl, wherein the alkyl, alkenyl, and alkynyl are independently substituted with 0, 1, 2, 3, or 4 R^{1A}; each R^{1A} is independently halogen, —OR^{1B}, or —N(R^{1B})₂; and each R^{1B} is independently hydrogen, C₁-C₃ alkyl or C₁-C₃ haloalkyl;
R² is hydrogen or C₁-C₆ alkyl substituted with 0, 1, 2, 3, or 4 R^{2A}, and each R^{2A} is independently halogen, —OR^{2B}, or —N(R^{2B})₂, wherein each R^{2B} is independently hydrogen. C₁-C₃ alkyl, or C₁-C₃ haloalkyl;
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, C₁-C₃ alkyl, or C₁-C₃ haloalkyl;
each R^{3E} is independently hydrogen, C₁-C₃ alkyl, or C₁-C₃ haloalkyl;
each R⁴ is independently halogen, —CN, -L₂-OR^{4A}, -L₂-N(R^{4B})₂, C₁-C₆ alkyl, or C₁-C₆ haloalkyl, wherein each R^{4A} and R^{4B} are independently hydrogen, C₁-C₃ alkyl, or C₁-C₃ haloalkyl;
each L₁ and L₂ is independently a bond, C₁-C₃ alkylene, or C₁-C₃ haloalkylene; and
m is 0, 1 or 2.

In some embodiments, the compound is a compound of Formula (I), or a pharmaceutically acceptable salt thereof, wherein:
R¹ is C₁-C₆ alkyl, substituted with 0, 1, 2, 3, or 4 R^{1A}; each R^{1A} is independently —OR^{1B}; and each R^{1B} is independently hydrogen or C₁-C₃ alkyl;
R² is hydrogen or C₁-C₆ alkyl substituted with 0, 1, 2, 3, or 4 R^{2A}, and each R^{2A} is independently —OR^{2B}, wherein each R^{2B} is independently hydrogen or C₁-C₃ alkyl;
R³ is C₁-C₁₀ alkyl, C₃-C₁₀ carbocyclyl, or 4- to 10-membered heterocyclyl, wherein the alkyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, halogen, ═O, -L₁-CN, -L₁-SO₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, carbocyclyl, heterocyclyl, aryl, and heteroaryl are independently substituted with 0, 1, 2, 3, or 4 R^{3D};
each R^{3C} is independently C₁-C₃ alkyl;
each R^{3D} is independently halogen or —OR^{3E};
R^{3E} is C₁-C₃ alkyl;
each R⁴ is independently halogen, —CN, -L₂-OR^{4A}, -L₂-N(R^{4B})₂, C₁-C₆ alkyl, or C₁-C₆ haloalkyl; each R^{4A} and R^{4B} are independently hydrogen, C₁-C₃ alkyl, or C₁-C₃ haloalkyl;
each L₁ and L₂ is independently a bond or C₁-C₃ alkylene; and
m is 1 or 2.

Applicants have found that compounds of Formula (I), comprising the combination of an —OR¹ group at the C₃ position of the pyrone ring, an amino moiety at the C₄ position of the pyrone ring, and a 5-membered monocyclic heteroaryl Ring A, show improvement in one or more desirable drug-like properties, such as improvement in unbound clearance, permeability, bioavailability, hcGAS potency and inhibitory activity, and/or solubility, compared to compounds absent that combination. Applicants have additionally found that incorporating an -L¹-OR^{3B} group, which is an exemplary substituent of group R³, may show additional improvements in one or more of these desirable properties. As a non-limiting example, as shown in Table D of the Examples, inclusion of —CH₂OCH₃ to Compound 103 provides Compound 114 with improved inhibitory activity in both the hcGAS Kinase glo and hcGAS LCMS assays.

In some embodiments, compounds of Formula (I) comprise at least one R^{3A} substituent -L₁-OR^{3B}, wherein L₁ is a bond, C₁-C₃ alkylene, or C₁-C₃ haloalkylene, and R³ is C₁-C₁₀, alkyl, C₂-C₁₀, alkenyl, C₂-C₁₀ alkynyl, C₃-C₁₀ carbocyclyl, or 4-10 membered heterocyclyl, wherein the alkyl, alkenyl, alkynyl, carbocyclyl, or heterocyclyl are independently substituted with 0, 1, 2, or 3 additional R^{3A} substituents.

For example, in some embodiments, the amino moiety

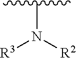

at the C₄ position of Formula (I) may be a group of formula (i-a), (ii-a), or (iii-a):

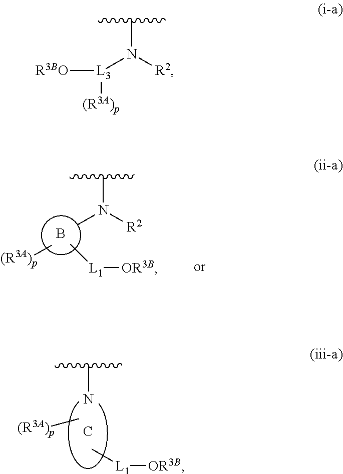

wherein L¹, R^{3A}, and R^{3B} are as defined herein:
   L₃ is C₁-C₁₀ alkylene, C₂-C₁₀ alkenylene, or C₂-C₁₀ alkynylene;
   Ring B is a C₃-C₁₀ carbocyclyl or 4- to 10-membered heterocyclyl;
   Ring C is a 5- to 10-membered heterocyclyl; and
   p is 0, 1, 2, or 3.

In some embodiments of Formula (I), the compound is of Formula (I′):

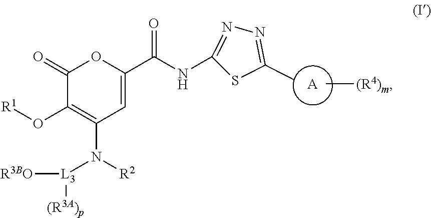

or a pharmaceutically acceptable salt thereof, wherein L₃ is C₁-C₁₀ alkylene, C₂-C₁₀ alkenylene, or C₂-C₁₀ alkynylene, and p is 0, 1, 2, or 3.

In some embodiments of Formula (I), the compound is of Formula (I″):

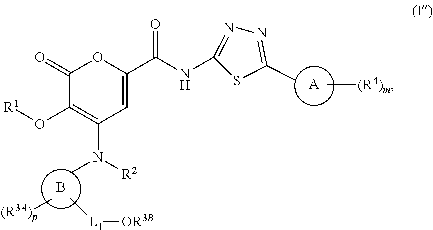

or a pharmaceutically acceptable salt thereof, wherein Ring B is C₃-C₁₀ carbocyclyl or 4- to 10-membered heterocyclyl, and p is 0, 1, 2, or 3.

In some embodiments of Formula (I), the compound is of Formula (I′″):

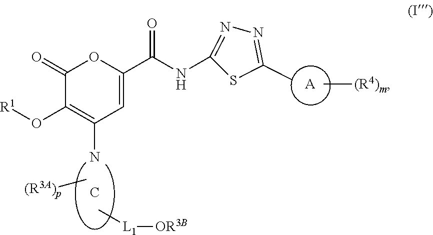

or a pharmaceutically acceptable salt thereof, wherein Ring C is a 5- to 10-membered heterocyclyl, and p is 0, 1, 2, or 3.

In some additional embodiments of Formula (I), the compound is of Formula (I″″):

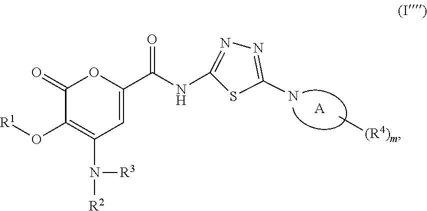

or a pharmaceutically acceptable salt thereof, wherein the nitrogen atom of the heteroaryl Ring A is directly linked to the thiadiazole moiety.

Additional embodiments are further described below and herein.

(a) R¹, R^{1A}, R^{1B}, R^{1C}, R^{1D}, R^{1E}, R^{1F}, R², R^{2A}, R^{2B}, x, R³, R^{3A}, R^{3B}, R^{3C}, R^{3D}, R^{3E}, L₁ and L₃

As generally described herein, R¹ is C₁-C₆ alkyl, C₂-C₆ alkenyl, C₁-C₆ alkynyl, -L₁-(C₁-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A};
each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O;
each R^{1B} is independently hydrogen, C₁-C₄alkyl, or C₁-C₄haloalkyl, wherein the alkyl and haloalkyl are independently substituted with 0, 1, 2, 3, or 4 R^{1D};
each R^{1C} is independently hydrogen, C₁-C₃alkyl, C₁-C₃haloalkyl, or —OR^{1F};
each R^{1D} is independently halogen, —OR^{1F}, or —N(R^{1F})₂; and
each R^{1F} is independently hydrogen, C₁-C₃alkyl, or C₁-C₃haloalkyl;
L₃ is a bond, C₁-C₃ alkylene, or —(C₁-C₃ alkylene)-O—, wherein the alkylene is independently substituted with 0, 1, 2, 3, or 4 R^{1E}; and
each R^{1E} is independently —(C₁-C₃ alkylene)-OR^{1B} or OR^{1B}, or two instances of R^{1E} are taken together to form ═O.

In some embodiments, R¹ is C₁-C₆ alkyl, C₂-C₆ alkenyl, or C₁-C₆ alkynyl, wherein the alkyl, alkenyl, and alkynyl are independently substituted with 0, 1, 2, 3, or 4 R^{1A}; each R^{1A} is independently halogen, —OR^{1B}, or —N(R^{1B})₂; and each R^{1B} is independently hydrogen, C₁-C₃alkyl or C₁-C₃haloalkyl.

In some embodiments, R¹ is C₁-C₆ alkyl substituted with 0, 1, 2, 3, or 4 R^{1A}. In some embodiments, R¹ is C₁-C₄ alkyl substituted with 0, 1, 2, 3, or 4 R^{1A}. In some embodiments, R¹ is C₁-C₃ alkyl substituted with 0, 1, 2, 3, or 4 R^{1A}. In some embodiments, R¹ is C₁-C₂ alkyl substituted with 0, 1, 2, 3, or 4 R^{1A}.

In some embodiments, R¹ is C₂-C₆ alkenyl substituted with 0, 1, 2, 3, or 4 R^{1A}. In some embodiments, R¹ is C₂-C₄ alkenyl substituted with 0, 1, 2, 3, or 4 R^{1A}. In some embodiments, R¹ is C₂-C₃ alkenyl substituted with 0, 1, 2, 3, or 4 R^{1A}.

In some embodiments, R¹ is C₂-C₆ alkynyl substituted with 0, 1, 2, 3, or 4 R^{1A}. In some embodiments, R¹ is C₂-C₄ alkynyl substituted with 0, 1, 2, 3, or 4 R^{1A}. In some embodiments, R¹ is C₂-C₃ alkynyl substituted with 0, 1, 2, or 3 R^{1A}.

In some embodiments, R¹ is C₁-C₆ alkyl substituted with 0 R^{1A}.

In some embodiments, R¹ is C₁-C₆ alkyl substituted with 1 R^{1A}; R^{1A} is halogen, —OR^{1B}, or —N(R^{1B})₂; and each R^{1B} is independently hydrogen, C₁-C₃ alkyl or C₁-C₃ haloalkyl.

In some embodiments, R¹ is C₁-C₆ alkyl substituted with 1 R^{1A}; R^{1A} is —OR^{1B}; and R^{1B} is hydrogen, C₁-C₃ alkyl or C₁-C₃ haloalkyl.

In some embodiments, R¹ is C₁-C₆ alkyl substituted with 1 R^{1A}; R^{1A} is —OR^{1B}; and R^{1B} is hydrogen.

In some embodiments, R¹ is C₁-C₆ alkyl substituted with 1 R^{1A}; R^{1A} is —OR^{1B}; and R^{1B} is C₁-C₃ alkyl.

In some embodiments, R¹ is C₁-C₆ alkyl substituted with 1 R^{1A}; R^{1A} is —C(═O)N(R^{1C})₂; and each R^{1C} is independently hydrogen or —OR^{1B}.

In some embodiments, R¹ is C₁-C₆ alkyl substituted with 1 R^{1A}; R^{1A} is —C(═O)N(R^{1C})₂; and one instance of R¹ is hydrogen, and the other is —OR^{1B}.

In some embodiments, R¹ is -L₃-(C₁-C₆ carbocyclyl), wherein the carbocyclyl is substituted with 0, 1, 2, 3, or 4 R^{1A}. In some embodiments, R¹ is -L₃-(C₃-C₄ carbocyclyl), wherein the carbocyclyl is substituted with 0, 1, 2, 3, or 4 R^{1A}. In some embodiments, R¹ is -L₃-(C₃ carbocyclyl), wherein the carbocyclyl is substituted with 0, 1, 2, 3, or 4 R^{1A}.

In some embodiments, R¹ is -L₃-(C₃-C₆ carbocyclyl), wherein the carbocyclyl is substituted with 1 R^{1A}; and R^{1A} is —OR^{1B}. In some embodiments, R¹ is -L₃-(C₃-C₄ carbocyclyl), wherein the carbocyclyl is substituted with 1 R^{1A}; and R^{1A} is —OR^{1B}. In some embodiments, R¹ is -L₃-(C₃ carbocyclyl), wherein the carbocyclyl is substituted with 1 R^{1A}; and R^{1A} is —OR^{1B}.

In some embodiments, R¹ is -L₃-(C₃-C₆ carbocyclyl), wherein the carbocyclyl is substituted with 1 R^{1A}; L₃ is C₁-C₃ alkylene; and R^{1A} is —OR^{1B}. In some embodiments, R¹ is -L₃-(C₃-C₄ carbocyclyl), wherein the carbocyclyl is substituted with 1 R^{1A}; L₃ is C₁-C₃ alkylene; and R^{1A} is —OR^{1B}. In some embodiments, R¹ is -L₃-(C₃ carbocyclyl), wherein the carbocyclyl is substituted with 1 R^{1A}; L₃ is C₁-C₃ alkylene; and R^{1A} is —OR^{1B}.

In some embodiments, when R¹ is -L₃-(C₃₋₆ carbocyclyl), the carbocyclyl ring is:

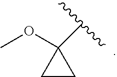 [SMILES: *C1(OC)CC1]

In some embodiments, R¹ is -L₃-(4- to 10-membered heterocyclyl), wherein the heterocyclyl is substituted with 0, 1, 2, 3, or 4 R^{1A}. In some embodiments, R¹ is -L₃-(5- to 6-membered heterocyclyl), wherein the heterocyclyl is substituted with 0, 1, 2, 3, or 4 R^{1A}. In some embodiments, R¹ is -L₃-(6-membered heterocyclyl), wherein the heterocyclyl is substituted with 0, 1, 2, 3, or 4 R^{1A}. In some embodiments, R¹ is -L₁-(5-membered heterocyclyl), wherein the heterocyclyl is substituted with 0, 1, 2, 3, or 4 R^{1A}.

In some embodiments, R¹ is -L₃-(4- to 10-membered heterocyclyl), wherein the heterocyclyl is substituted with 0 R^{1A}; and L₃ is C₁-C₃ alkylene, wherein the alkylene is substituted with 0 R^{1E}. In some embodiments, R¹ is -L₃-(5- to 6-membered heterocyclyl), wherein the heterocyclyl is substituted with 0 R^{1A}; and L₃ is C₁-C₃ alkylene, wherein the alkylene is substituted with 0 R^{1E}. In some embodiments, R¹ is -L₃-(6-membered heterocyclyl), wherein the heterocyclyl is substituted with 0 R^{1A}; and L₃ is C₁-C₃ alkylene, wherein the alkylene is substituted with 0 R^{1E}.

In some embodiments, R¹ is -L₃-(4- to 10-membered heterocyclyl), wherein the heterocyclyl is substituted with 0 R^{1A}; and L₃ is a bond. In some embodiments, R¹ is -L₃-(5- to 6-membered heterocyclyl), wherein the heterocyclyl is substituted with 0 R^{1A}; and L₃ is a bond. In some embodiments, R¹ is -L₃-(5-membered heterocyclyl), wherein the heterocyclyl is substituted with 0 R^{1A}, and L₃ is a bond.

In some embodiments, when R¹ is -L₃-(4- to 10-membered heterocyclyl), the heterocyclyl ring comprises 1, 2, or 3 ring heteroatoms independently selected from O, N, and S. In some embodiments, when R¹ is -L₁-(4- to 10-membered heterocyclyl), the heterocyclyl ring comprises 1 or 2 ring heteroatoms independently selected from O, N, and S.

In some embodiments, when R¹ is -L₃-(7- to 10-membered heterocyclyl), the heterocyclyl ring comprises 1, 2, or 3 ring heteroatoms independently selected from O, N, and S. In some embodiments, when R¹ is -L₃-(7- to 10-membered heterocyclyl), the heterocyclyl ring comprises 1 or 2 ring heteroatoms independently selected from O, N, and S.

In some embodiments, when R¹ is -L₃-(4- to 6-membered heterocyclyl), the heterocyclyl ring comprises 1, 2, or 3 ring heteroatoms independently selected from O, N, and S. In some embodiments, when R¹ is -L₃-(4- to 6-membered heterocyclyl), the heterocyclyl ring comprises 1 or 2 ring heteroatoms independently selected from O, N, and S.

In some embodiments, when R¹ is -L₃-(5- to 6-membered heterocyclyl), the heterocyclyl ring comprises 1, 2, or 3 ring heteroatoms independently selected from O, N, and S. In some embodiments, when R¹ is -L₃-(5- to 6-membered heterocyclyl), the heterocyclyl ring comprises 1 or 2 ring heteroatoms independently selected from O, N, and S.

In some embodiments, when R¹ is -L₃-(6-membered heterocyclyl), the heterocyclyl ring comprises 1, 2, or 3 ring heteroatoms independently selected from O, N, and S. In some embodiments, when R¹ is -L₃-(6-membered heterocyclyl), the heterocyclyl ring comprises 1 or 2 ring heteroatoms independently selected from O, N, and S.

In some embodiments, when R¹ is -L₃-(5-membered heterocyclyl), the heterocyclyl ring comprises 1 or 2 ring heteroatoms independently selected from O, N, and S. In some embodiments, when R¹ is -L₃-(5-membered heterocyclyl), the heterocyclyl ring comprises 1 ring 0 atom.

In some embodiments, when R¹ is -L₃-(4- to 10-membered heterocyclyl), the heterocyclyl ring is selected from:

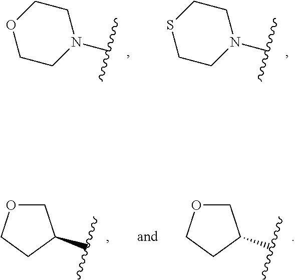 [SMILES: *N1CCOCC1 ; *N1CCSCC1 ; *[C@H]1CCOC1 ; *[C@@H]1CCOC1]

As generally described herein, L₃ is a bond, C₁-C₃ alkylene, or —(C₁-C₃ alkylene)-O—, wherein the alkylene is independently substituted with 0, 1, 2, 3, or 4 R^{1E}.

In some embodiments, L₃ is a bond.

In some embodiments, L₃ is C₁-C₃ alkylene independently substituted with 0, 1, 2, 3, or 4 R^{1E}. In some embodiments, L₃ is C₁-C₂ alkylene independently substituted with 0, 1, 2, 3, or 4 R^{1E}. In some embodiments, L₃ is C₁ alkylene independently substituted with 0, 1, or 2 R^{1E}.

In some embodiments, L₃ is C₁-C₃ alkylene independently substituted with 0 R^{1E}. In some embodiments, L₃ is C₁-C₂ alkylene independently substituted with 0 R^{1E}. In some embodiments, L₃ is C₁ alkylene substituted with 0 R^{1E}.

In some embodiments, at least one R^{1A} is independently halogen, —OR^{1B}, or —N(R^{1B})₂.

In some embodiments, at least one R^{1A} is independently halogen.

In some embodiments, at least one R^{1A} is independently —OR^{1B} or —N(R^{1B})₂.

In some embodiments, at least one R^{1A} is independently —OR^{1B}.

In some embodiments, at least one R^{1A} is independently —OH. In some embodiments, at least one R^{1A} is independently —O(C₁-C₃ alkyl).

In some embodiments, at least one R^{1A} is independently —N(R^{1B})₂.

In some embodiments, at least one instance of R^{1A} is —C(═O)OR^{1B}.

In some embodiments, at least one instance of R^{1A} is —C(═O)N(R^{1C})₂.

In some embodiments, at least one R^{1B} is independently hydrogen, C₁-C₃ alkyl or C₁-C₃ haloalkyl.

In some embodiments, at least one R^{1B} is independently hydrogen.

In some embodiments, at least one R^{1B} is independently C₁-C₃ alkyl or C₁-C₃ haloalkyl.

In some embodiments, at least one R^{1B} is independently C₁-C₃ alkyl.

In some embodiments, at least one R^{1B} is independently C₁-C₃ haloalkyl.

In some embodiments, at least one R^{1C} is independently hydrogen.

In some embodiments, at least one R^{1C} is independently C₁-C₃alkyl.

In some embodiments, at least one R^{1C} is independently C₁-C₃haloalkyl.

In some embodiments, at least one R^{1C} is independently —OR^{1B}. In some embodiments, at least one R^{1C} is independently —OCH₃.

In some embodiments, R¹ is —CH₃, —CH₂—C(CH₃)₂—CH₂OCH₃, —CH₂CH₂OH, or —CH₂CH₂OCH₃.

In some embodiments, R¹ is —CH₃. In some embodiments, R¹ is —CH₂—C(CH₃)₂—CH₂OCH₃. In some embodiments, R¹ is —CH₂CH₂OH. In some embodiments, R¹ is —CH₂CH₂OCH₃.

In some embodiments, R¹ is selected from:

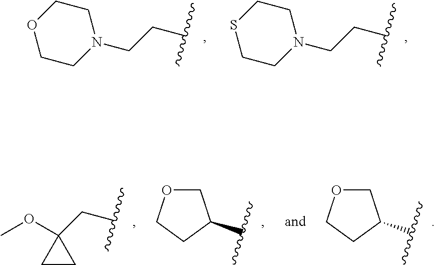

As generally defined herein, R² is hydrogen or C₁-C₆ alkyl substituted with 0, 1, 2, 3, or 4 R^{2A}, each R^{2A} is independently halogen, —OR^{2B}, or —N(R^{2B})₂, wherein each R^{2B} is independently hydrogen, C₁-C₃ alkyl, or C₁-C₃ haloalkyl; R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A}; or R² and R³ are joined, with the atom to which they are attached, to form a 4- to 10-membered heterocyclyl independently substituted with 0, 1, 2, 3, or 4 R^{3A}.

In some embodiments, R² is hydrogen or C₁-C₆ alkyl, wherein the alkyl is independently substituted with 0, 1, 2, 3, or 4 R^{2A}, and each R^{2A} is independently halogen, —OR^{2B}, or —N(R^{2B})₂, wherein each R^{2B} is independently hydrogen, C₁₋₃ alkyl, or C₁₋₃ haloalkyl.

In some embodiments, R² is hydrogen or C₁-C₆ alkyl substituted with 0, 1, 2, 3, or 4 R^{2A}.

In some embodiments, R² is hydrogen. In some embodiments, R² is C₁-C₆ alkyl substituted with 0, 1, 2, 3, or 4 R^{2A}.

In some embodiments, R² is C₁-C₆ alkyl substituted with 0 R^{2A}.

In some embodiments, R² is C₁-C₆ alkyl substituted with 1 R^{2A}.

In some embodiments, R² is C₁-C₆ alkyl substituted with 2 R^{2A}. In some embodiments, R² is C₁-C₆ alkyl substituted with 3 R^{2A}. In some embodiments, R² is C₁-C₆ alkyl substituted with 4 R^{2A}.

In some embodiments, at least one R^{2A} is independently halogen, —OR^{2B}, or —N(R^{2B})₂.

In some embodiments, at least one R^{2A} is independently halogen.

In some embodiments, at least one R^{2A} is independently —OR^{2B}.

In some embodiments, at least one R^{2A} is independently —OH.

In some embodiments, at least one R^{2A} is independently —O(C₁-C₃ alkyl).

In some embodiments, at least one R^{2A} is independently —O(CH₃).

In some embodiments, at least one R^{2A} is independently —N(R^{2B})₂.

In some embodiments, at least one R^{2B} is independently hydrogen, C₁-C₃ alkyl, or C₁-C₃ haloalkyl.

In some embodiments, at least one R^{2B} is independently hydrogen.

In some embodiments, at least one R^{2B} is independently C₁-C₃ alkyl or C₁-C₃ haloalkyl.

In some embodiments, at least one R^{2B} is independently C₁-C₃ alkyl.

In some embodiments, at least one R^{2B} is independently methyl. In some embodiments, at least one R^{2B} is independently ethyl. In some embodiments, at least one R^{2B} is independently propyl.

In some embodiments, at least one R¹ is independently C₁-C₃ haloalkyl.

In some embodiments, R¹ and R² are joined, with the atoms to which they are attached, to form a 6- or 7-membered heterocyclyl independently substituted with 0, 1, 2, 3, or 4 R^{1A}.

In some embodiments, R¹ and R² are joined, with the atoms to which they are attached, to form a 6-membered heterocyclyl independently substituted with 0, 1, 2, 3, or 4 R^{1A}.

In some embodiments, R¹ and R² are joined, with the atoms to which they are attached, to form a 6-membered heterocyclyl substituted with 0 R^{1A}. In some embodiments, R¹ and R² are joined, with the atoms to which they are attached, to form a 6-membered heterocyclyl substituted with 1 R^{1A}. In some embodiments, R¹ and R² are joined, with the atoms to which they are attached, to form a 6-membered heterocyclyl substituted with 2 R^{1A}. In some embodiments, R¹ and R² are joined, with the atoms to which they are attached, to form a 6-membered heterocyclyl substituted with 3 R^{1A}. In some embodiments, R¹ and R² are joined, with the atoms to which they are attached, to form a 6-membered heterocyclyl substituted with 4 R^{1A}.

In some embodiments, R¹ and R² are joined, with the atoms to which they are attached, to form:

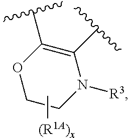

wherein x is 0, 1, 2, 3, or 4. In some embodiments, R¹ and R² are joined, with the atoms to which they are attached, to form:

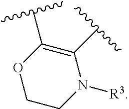

In some embodiments, R¹ and R² are joined, with the atoms to which they are attached, to form:

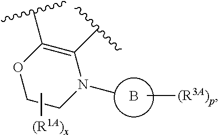

wherein x is 0, 1, 2, 3, or 4. In some embodiments, R¹ and R² are joined, with the atoms to which they are attached, to form:

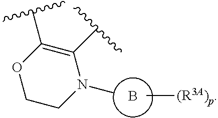

As generally defined herein, x is 0, 1, 2, 3, or 4. In some embodiments, x is 0, 1, 2, or 3. In some embodiments, x is 0. In some embodiments, x is 1. In some embodiments, x is 2. In some embodiments, x is 3. In some embodiments, x is 4.

In some embodiments, R³ is C₁-C₁₀ alkyl, C₂-C₁₀ alkenyl, C₂-C₁₀ alkynyl, C₃-C₁₀ carbocyclyl, or 4-10 membered heterocyclyl, wherein the alkyl, alkenyl, alkynyl, carbocyclyl, or heterocyclyl are independently substituted with 0, 1, 2, 3, or 4 R^{3A}.

In some embodiments, R³ is C₁-C₁₀ alkyl, C₃-C₁₀ carbocyclyl, or 4-10 membered heterocyclyl, wherein the alkyl, carbocyclyl, or heterocyclyl are independently substituted with 0, 1, 2, 3, or 4 R^{3A}.

In some embodiments, R³ is C₁-C₁₀ alkyl substituted with 0, 1, 2, 3, or 4 R^{3A}.

In some embodiments, R³ is C₁-C₁₀ alkyl.

In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is tert-butyl.

In some embodiments, R³ is C₁-C₁₀ alkyl substituted with 1 R^{3A}.

In some embodiments, R³ is methyl substituted with 1 R^{3A}. In some embodiments, R³ is ethyl substituted with 1 R^{3A}. In some embodiments, R³ is propyl substituted with 1 R^{3A}. In some embodiments, R³ is isopropyl substituted with 1 R^{3A}. In some embodiments, R³ is butyl substituted with 1 R^{3A}. In some embodiments, R³ is isobutyl substituted with 1 R^{3A}. In some embodiments, R³ is tert-butyl substituted with 1 R^{3A}.

In some embodiments, R³ is C₁-C₁₀ alkyl substituted with 2 R^{3A}.

In some embodiments, R³ is methyl substituted with 2 R^{3A}. In some embodiments, R³ is ethyl substituted with 2 R^{3A}. In some embodiments, R³ is propyl substituted with 2 R^{3A}. In some embodiments, R³ is isopropyl substituted with 2 R^{3A}. In some embodiments, R³ is butyl substituted with 2 R^{3A}. In some embodiments, R³ is isobutyl substituted with 2 R^{3A}. In some embodiments, R³ is tert-butyl substituted with 2 R^{3A}.

In some embodiments, R³ is C₁-C₁₀ alkyl substituted with 3 R^{3A}.

In some embodiments, R³ is methyl substituted with 3 R^{3A}. In some embodiments, R³ is ethyl substituted with 3 R^{3A}. In some embodiments, R³ is propyl substituted with 3 R^{3A}. In some embodiments, R³ is isopropyl substituted with 3 R^{3A}. In some embodiments, R³ is butyl substituted with 3 R^{3A}. In some embodiments, R³ is isobutyl substituted with 3 R^{3A}. In some embodiments, R³ is tert-butyl substituted with 3 R^{3A}.

In some embodiments, R³ is C₁-C₁₀ alkyl substituted with 4 R^{3A}.

In some embodiments, R³ is C₃-C₁₀ carbocyclyl substituted with 0, 1, 2, 3, or 4 R^{3A}.

In some embodiments, R³ is C₃-C₁₀ carbocyclyl.

In some embodiments, R³ is a fused C₃-C₁₀ carbocyclyl. In some embodiments, R³ is a spiro C₃-C₁₀ carbocyclyl. In some embodiments, R³ is a bridged C₃-C₁₀ carbocyclyl.

In some embodiments, R³ is C₃ carbocyclyl. In some embodiments, R³ is C₄ carbocyclyl. In some embodiments, R³ is C₅ carbocyclyl. In some embodiments, R³ is C₆ carbocyclyl. In some embodiments, R³ is C₇ carbocyclyl. In some embodiments, R³ is C₈ carbocyclyl. In some embodiments, R³ is C₉ carbocyclyl. In some embodiments, R³ is C₁₀ carbocyclyl.

In some embodiments, R³ is C₃-C₁₀ carbocyclyl substituted with 1 R^{3A}.

In some embodiments, R³ is C₃ carbocyclyl substituted with 1 R^{3A}. In some embodiments, R³ is C₄ carbocyclyl substituted with 1 R^{3A}. In some embodiments, R³ is C₅ carbocyclyl substituted with 1 R^{3A}. In some embodiments, R³ is C₆ carbocyclyl substituted with 1 R^{3A}. In some embodiments, R³ is C₇ carbocyclyl substituted with 1 R^{3A}. In some embodiments, R³ is C₈ carbocyclyl substituted with 1 R^{3A}. In some embodiments, R³ is C₄ carbocyclyl substituted with 1 R^{3A}. In some embodiments, R³ is C₁₀ carbocyclyl substituted with 1 R^{3A}.

In some embodiments, R³ is C₃-C₁₀ carbocyclyl substituted with 2 R^{3A}.

In some embodiments, R³ is C₃ carbocyclyl substituted with 2 R^{3A}. In some embodiments, R³ is C₄ carbocyclyl substituted with 2 R^{3A}. In some embodiments, R³ is C₃ carbocyclyl substituted with 2 R^{3A}. In some embodiments, R³ is C₆ carbocyclyl substituted with 2 R^{3A}. In some embodiments, R³ is C₇ carbocyclyl substituted with 2 R^{3A}. In some embodiments, R³ is C₈ carbocyclyl substituted with 2 R^{3A}. In some embodiments, R³ is C₉ carbocyclyl substituted with 2 R^{3A}. In some embodiments, R³ is C₁₀ carbocyclyl substituted with 2 R^{3A}.

In some embodiments, R³ is C₃-C₁₀ carbocyclyl substituted with 3 R^{3A}.

In some embodiments, R³ is C₃ carbocyclyl substituted with 3 R^{3A}. In some embodiments, R³ is C₄ carbocyclyl substituted with 3 R^{3A}. In some embodiments, R³ is C₃ carbocyclyl substituted with 3 R^{3A}. In some embodiments, R³ is C₆ carbocyclyl substituted with 3 R^{3A}. In some embodiments, R³ is C₇ carbocyclyl substituted with 3 R^{3A}. In some embodiments, R³ is C₈ carbocyclyl substituted with 3 R^{3A}. In some embodiments, R³ is C₉ carbocyclyl substituted with 3 R^{3A}. In some embodiments, R³ is C₁₀ carbocyclyl substituted with 3 R^{3A}.

In some embodiments, R³ is C₃-C₁₀ carbocyclyl substituted with 4 R^{3A}.

In some embodiments, R³ is 4- to 10-membered heterocyclyl substituted with 0, 1, 2, 3, or 4 R^{3A}.

In some embodiments, R³ is 4- to 10-membered heterocyclyl.

In some embodiments, R³ is a fused 6- to 10-membered heterocyclyl. In some embodiments, R³ is a spiro 6- to 10-membered heterocyclyl. In some embodiments, R³ is a bridged 4- to 10-membered heterocyclyl.

In some embodiments, R³ is 4-membered heterocyclyl. In some embodiments, R³ is 5-membered heterocyclyl. In some embodiments, R³ is 6-membered heterocyclyl. In some embodiments, R³ is 7-membered heterocyclyl. In some embodiments, R³ is 8-membered heterocyclyl. In some embodiments, R³ is 9-membered heterocyclyl. In some embodiments, R³ is 10-membered heterocyclyl.

In some embodiments, R³ is 4- to 10-membered heterocyclyl substituted with 1 R^{3A}.

In some embodiments, R³ is 4-membered heterocyclyl substituted with 1 R^{3A}. In some embodiments, R³ is 5-membered heterocyclyl substituted with 1 R^{3A}. In some embodiments, R³ is 6-membered heterocyclyl substituted with 1 R^{3A}. In some embodiments, R³ is 7-membered heterocyclyl substituted with 1 R^{3A}. In some embodiments, R³ is 8-membered heterocyclyl substituted with 1 R^{3A}. In some embodiments, R³ is 9-membered heterocyclyl substituted with 1 R^{3A}. In some embodiments, R³ is 10-membered heterocyclyl substituted with 1 R^{3A}.

In some embodiments, R³ is 4- to 10-membered heterocyclyl substituted with 2 R^{3A}.

In some embodiments, R³ is 4-membered heterocyclyl substituted with 2 R^{3A}. In some embodiments, R³ is 5-membered heterocyclyl substituted with 2 R^{3A}. In some embodiments, R³ is 6-membered heterocyclyl substituted with 2 R^{3A}. In some embodiments, R³ is 7-membered heterocyclyl substituted with 2 R^{3A}. In some embodiments, R³ is 8-membered heterocyclyl substituted with 2 R^{3A}. In some embodiments, R³ is 9-membered heterocyclyl substituted with 2 R^{3A}. In some embodiments, R³ is 10-membered heterocyclyl substituted with 2 R^{3A}.

In some embodiments, R³ is 4- to 10-membered heterocyclyl substituted with 3 R^{3A}.

In some embodiments, R³ is 4-membered heterocyclyl substituted with 3 R^{3A}. In some embodiments, R³ is 5-membered heterocyclyl substituted with 3 R^{3A}. In some embodiments, R³ is 6-membered heterocyclyl substituted with 3 R^{3A}. In some embodiments, R³ is 7-membered heterocyclyl substituted with 3 R^{3A}. In some embodiments, R³ is 8-membered heterocyclyl substituted with 3 R^{3A}. In some embodiments, R³ is 9-membered heterocyclyl substituted with 3 R^{3A}. In some embodiments, R³ is 10-membered heterocyclyl substituted with 3 R^{3A}.

In some embodiments, R³ is 4- to 10-membered heterocyclyl substituted with 4 R^{3A}.

In certain embodiments, R³ is

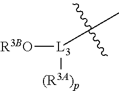

wherein L₃ is C₁-C₁₀ alkylene, C₂-C₁₀ alkenylene, or C₂-C₁₀ alkynylene, and p is 0, 1, 2, or 3.

In certain embodiments, R³ is

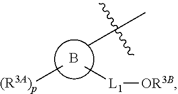

wherein Ring B is the C₃-C₁₀ carbocyclyl or 4- to 10-membered heterocyclyl, and p is 0, 1, 2, or 3.

In certain embodiments, R³ is —CH₃, —CH₂CH₃, —CH₂CHF₂, —CH₂CF₃, —CH(CH₃)₂,

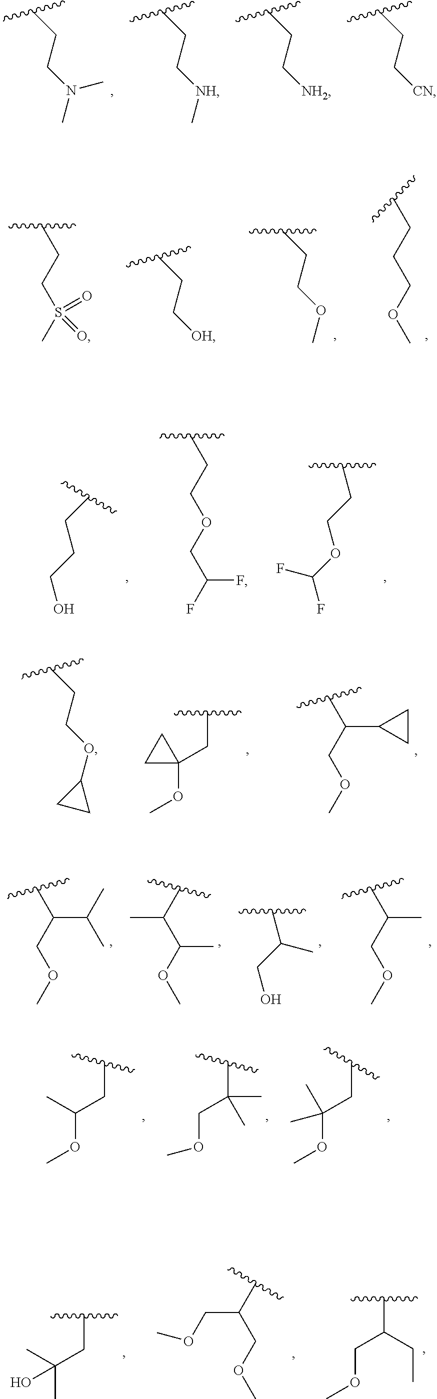

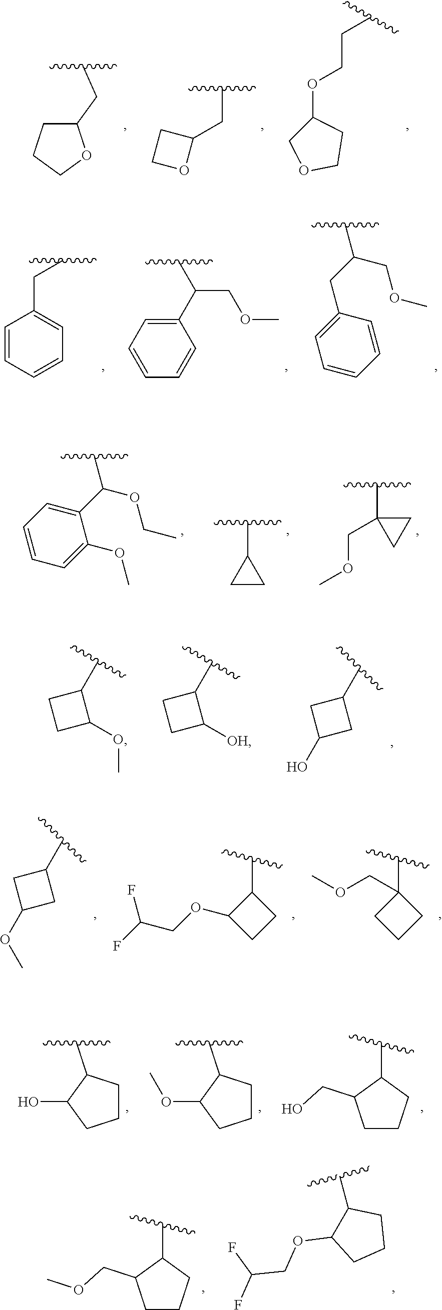

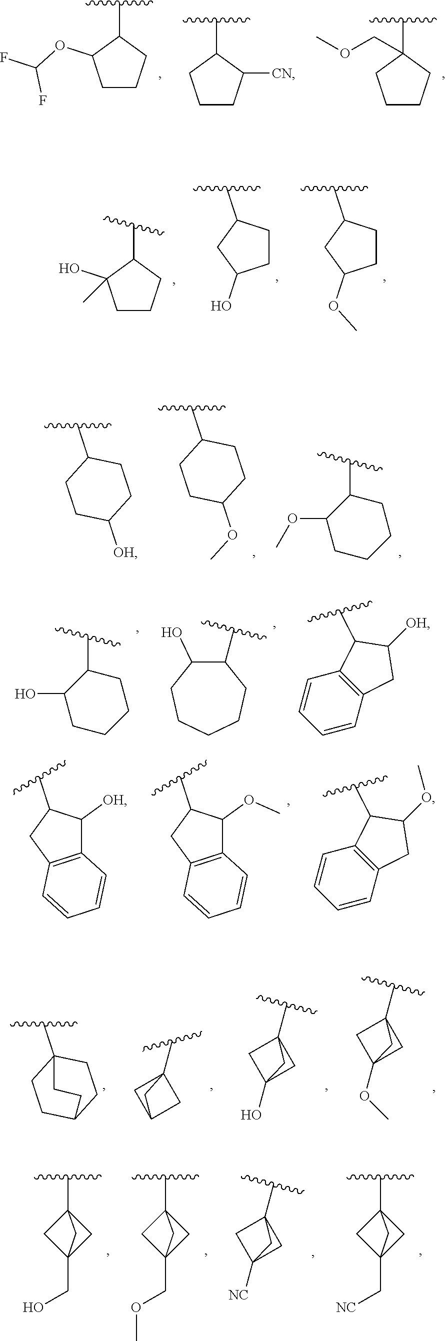

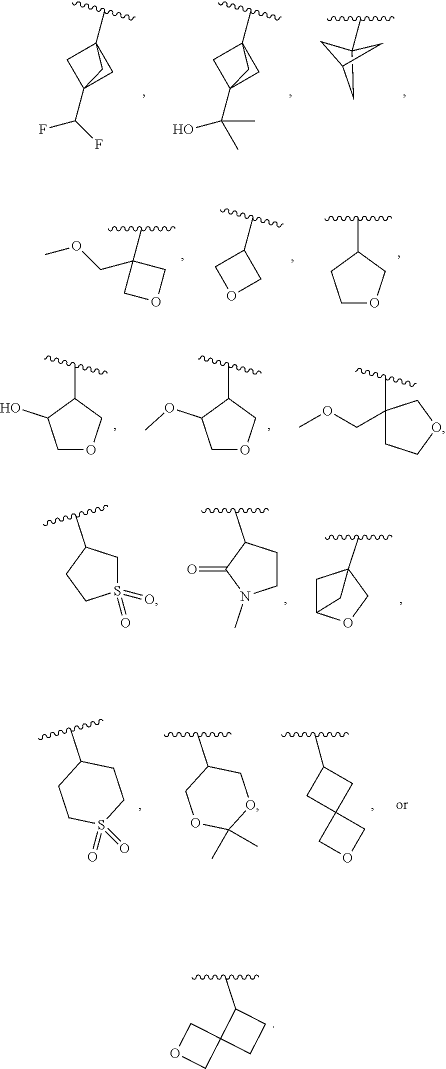

In some embodiments, R² is hydrogen or C₁-C₆ alkyl, and R³ is C₁-C₁₀ alkyl.

In some embodiments, the amino moiety

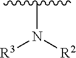

is selected from the group consisting of:

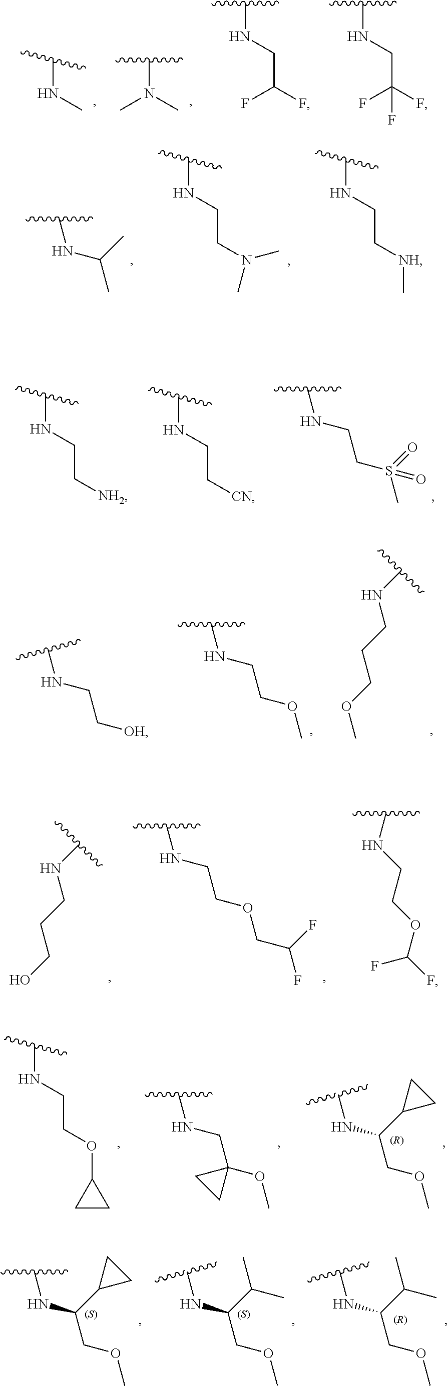

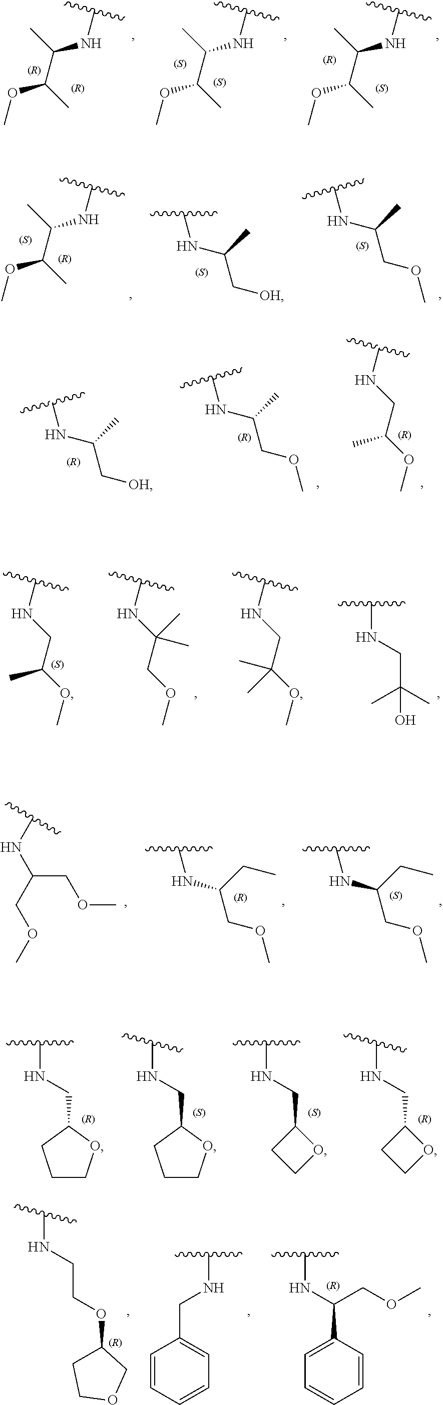

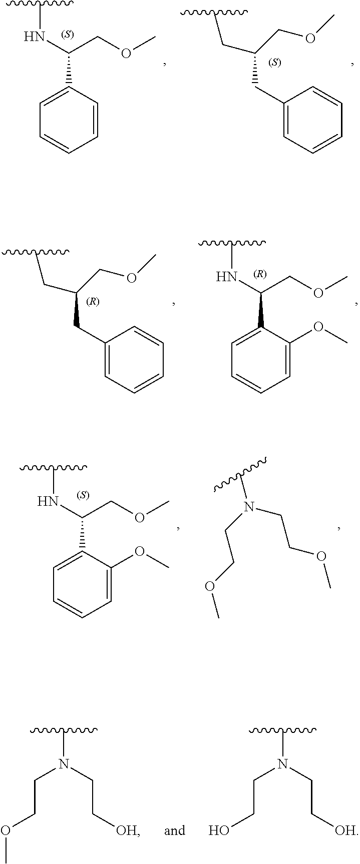

In some embodiments, the amino moiety

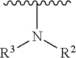

is selected from the group consisting of:

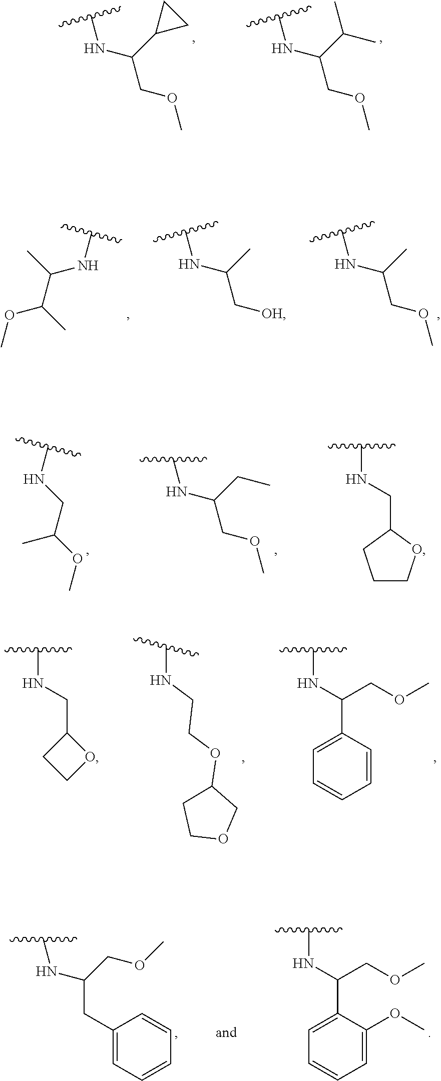

In some embodiments, the amino moiety

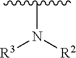

is selected from the group consisting of:

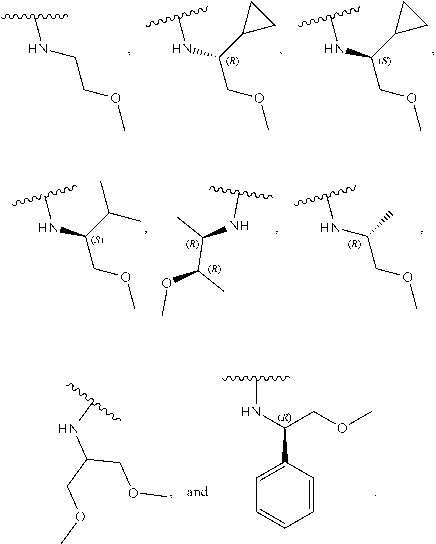

In some embodiments,

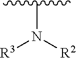

is

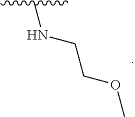 [SMILES: *NCCOC]

In some embodiments,

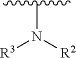

is

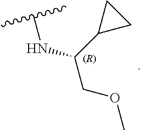

In some embodiments,

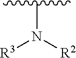

is

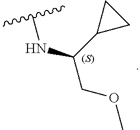

In some embodiments,

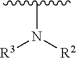

is

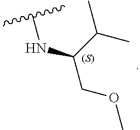

In some embodiments,

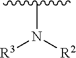

is

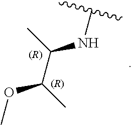

In some embodiments,

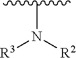

is

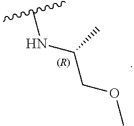

In some embodiments,

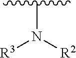

is

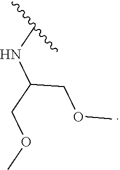

In some embodiments,

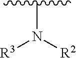

is

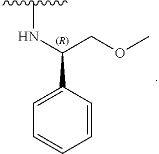

In some embodiments, group R³ is of formula

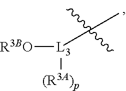

wherein L₃ is C₁-C₁₀ alkylene, C₁-C₁₀ alkenylene, or C₂-C₁₀ alkynylene, and p is 0, 1, 2, or 3.

In some embodiments, R² is hydrogen or C₁-C₆ alkyl, and R³ is

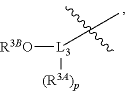

to provide an amino moiety of formula (i-a):

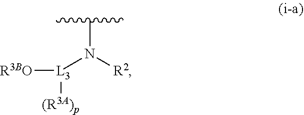

wherein L₃ is C₁-C₁₀ alkylene, C₂-C₁₀ alkenylene, or C₂-C₁₀ alkynylene, and p is 0, 1, 2, or 3.

In some embodiments, L₃ is C₁-C₁₀ alkylene, C₂-C₁₀ alkenylene, or C₂-C₁₀ alkynylene.

In some embodiments, L₃ is C₁-C₁₀ alkylene. In some embodiments, L₃ is C₁-C₆ alkylene. In some embodiments, L₃ is C₁-C₄ alkylene. In some embodiments, L₃ is C₁-C₃ alkylene.

In some embodiments, L₃ is C₂-C₁₀ alkenylene. In some embodiments, L₃ is C₂-C₁₀ alkynylene.

In some embodiments, amino moieties of formula

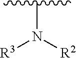

which fall within the scope of formula (i-a) include, but are not limited to:

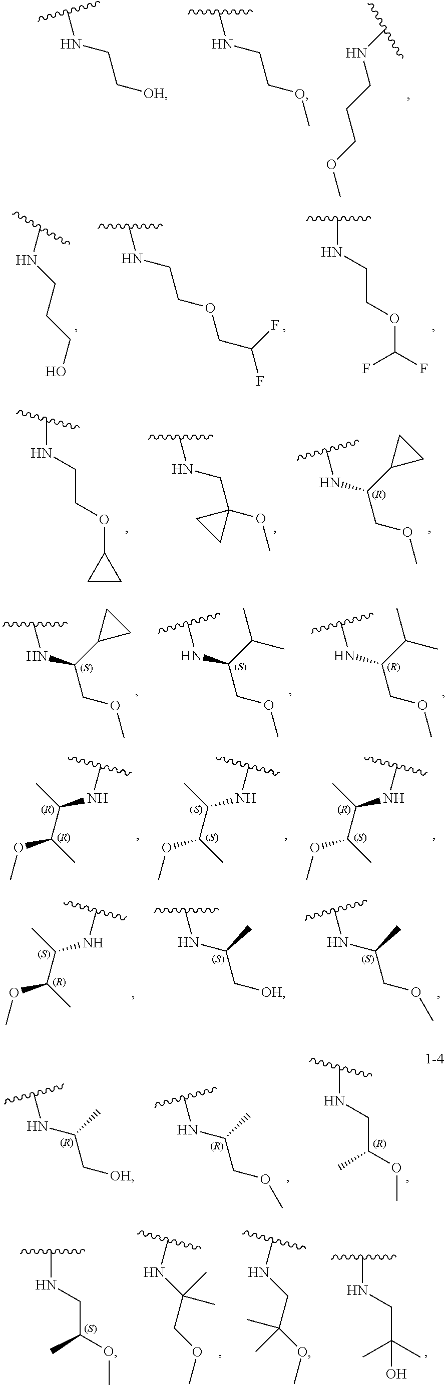

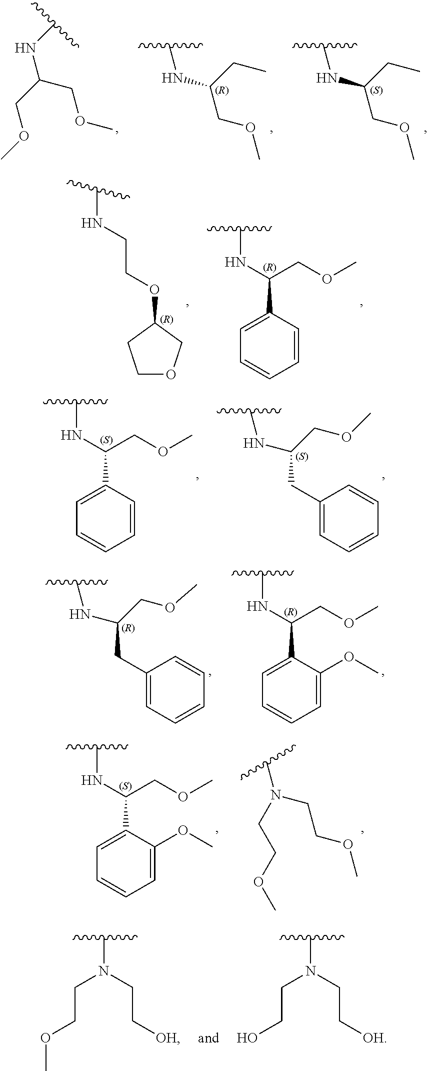

In some embodiments, amino moieties of formula

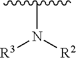

which fall within the scope of formula (i-a) include, but are not limited to:

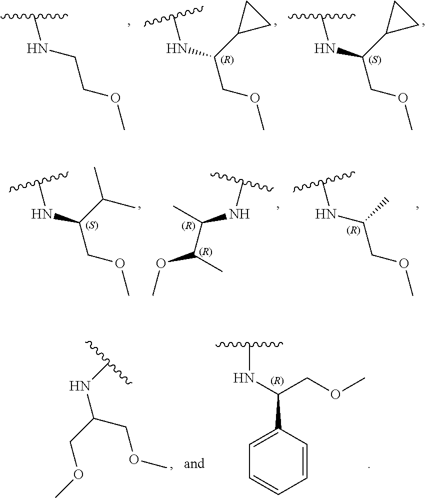

In some embodiments, R² is hydrogen or C₁-C₆ alkyl, and R³ is C₁-C₁₀ carbocyclyl.

In some embodiments, the amino moiety

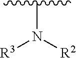

is selected from the group consisting of:

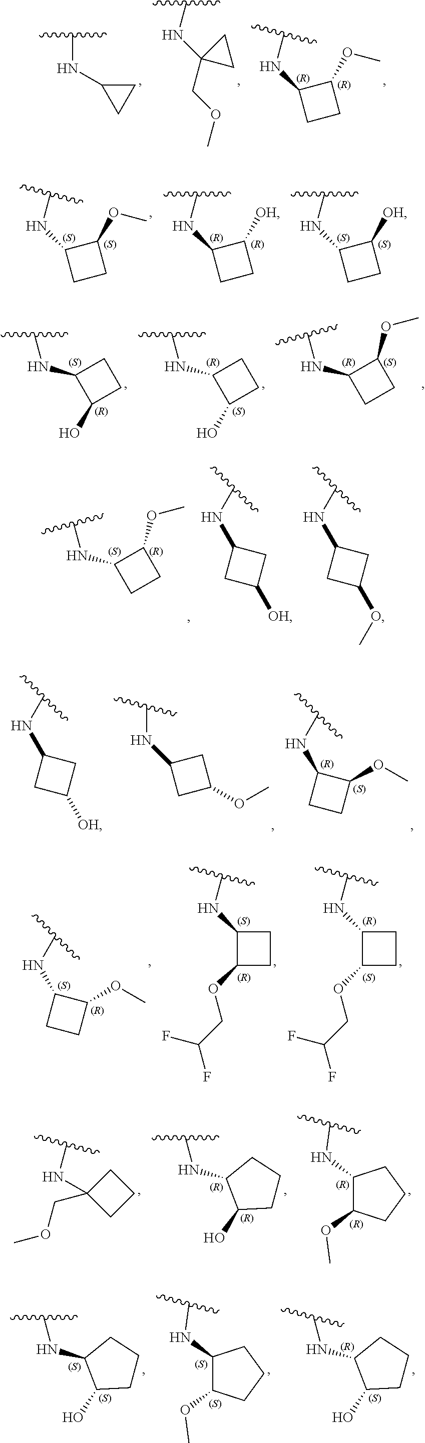

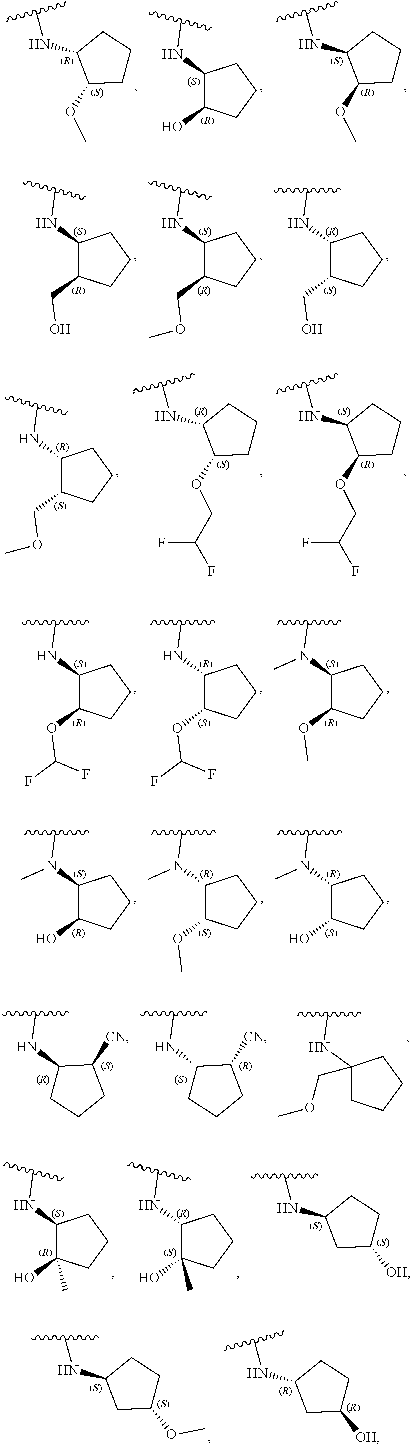

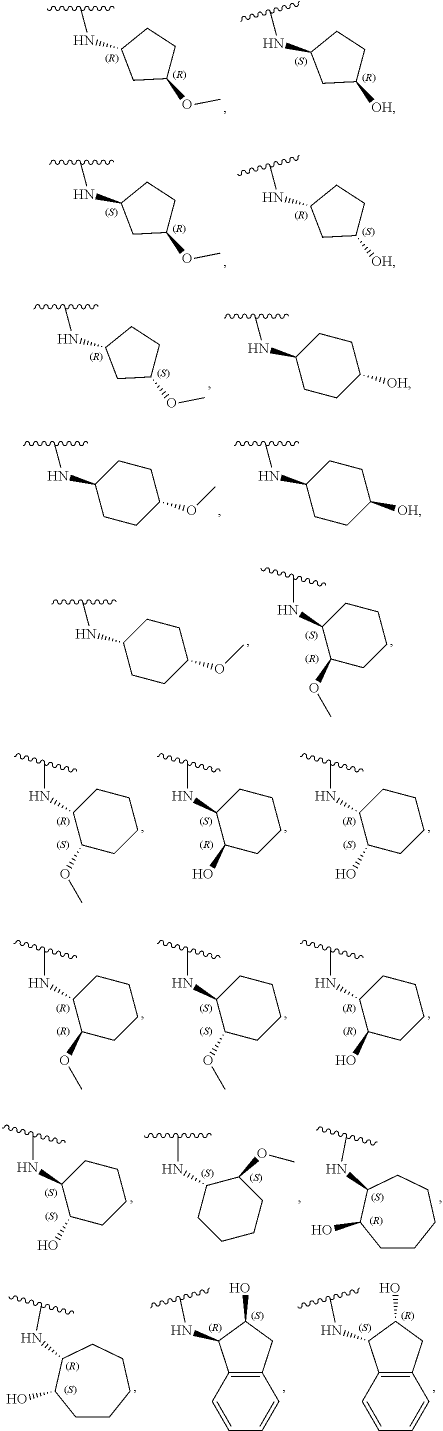

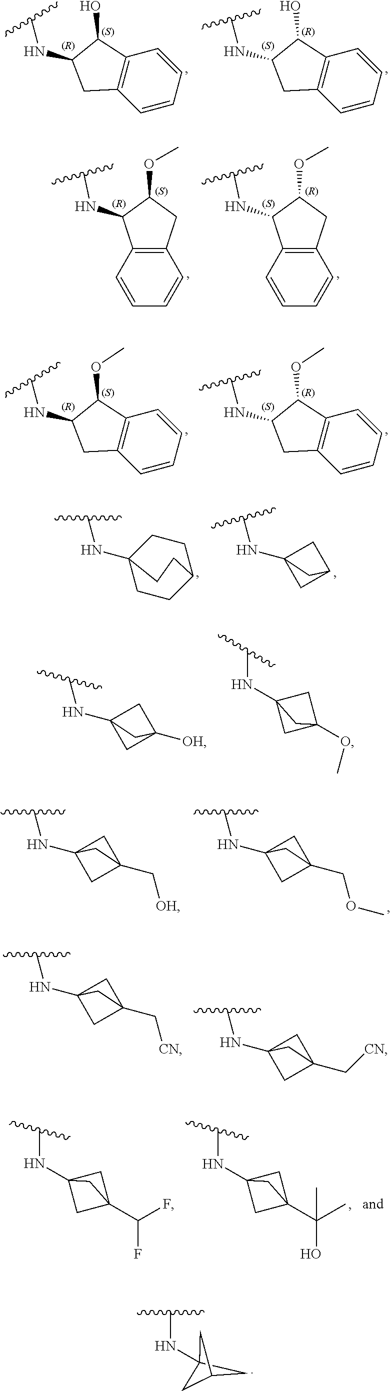

In some embodiments, the amino moiety

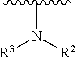

selected from the group consisting of:

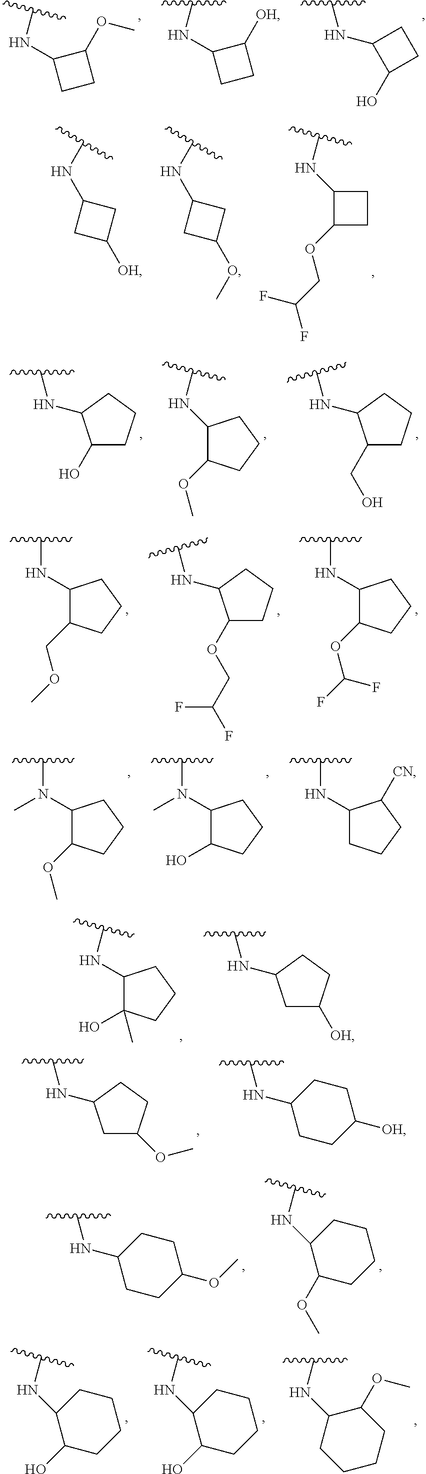

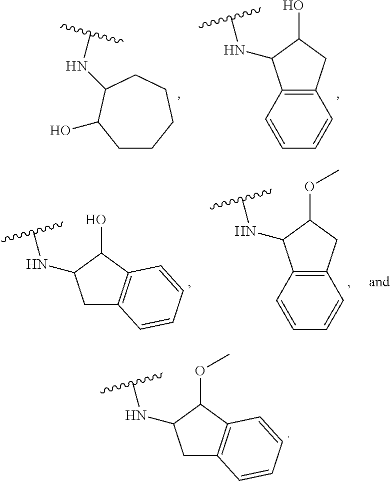

In some embodiments, the amino moiety

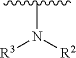

is selected from the group consisting of:

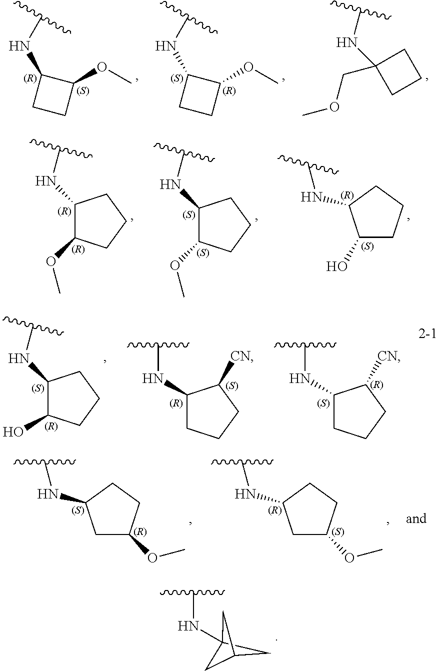

In some embodiments,

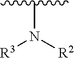

is

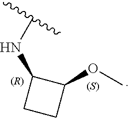

In some embodiments,

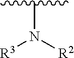

is

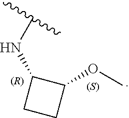

In some embodiments,

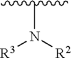

is

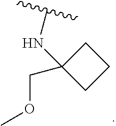 [SMILES: *NC1(COC)CCC1]

In some embodiments,

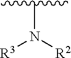

is

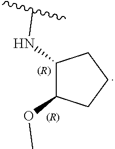

In some embodiments,

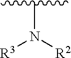

is

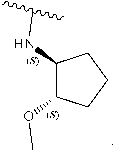

In some embodiments,

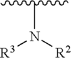

is

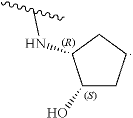

In some embodiments,

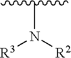

is

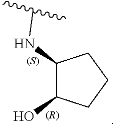

In some embodiments,

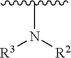

is

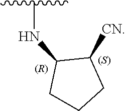

In some embodiments,

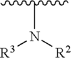

is

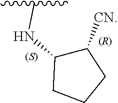

In some embodiments,

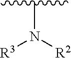

In some embodiments,

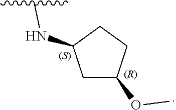

In some embodiments,

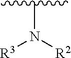

is

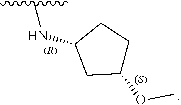

In some embodiments,

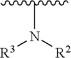

is

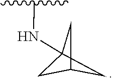 [SMILES: *NC12CC(C1)C2]

In some embodiments, R² is hydrogen or C₁-C₆ alkyl, and R³ is 4-10 membered heterocyclyl.

In some embodiments, the amino moiety

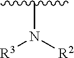

is selected from the group consisting of:

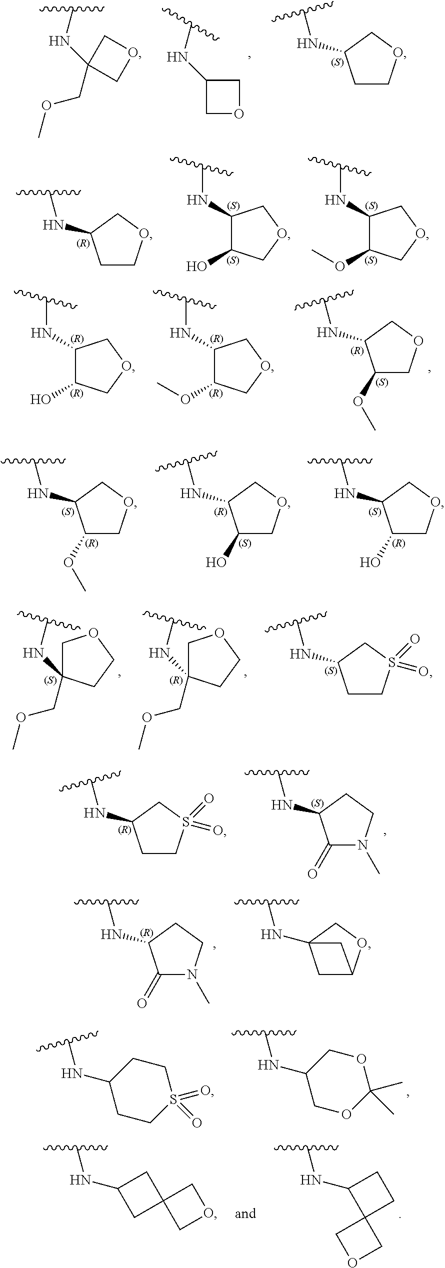

In some embodiments, the amino moiety

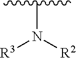

is selected from the group consisting of:

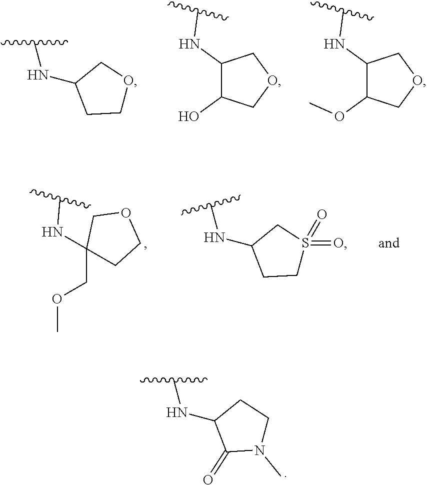

In some embodiments, R³ is

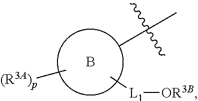

wherein Ring B is the C₃-C₁₀ carbocyclyl or 4- to 10-membered heterocyclyl, and p is 0, 1, 2, or 3.

In some embodiments, R² is hydrogen or C₁-C₆ alkyl and R³ is

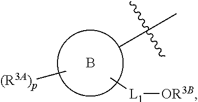

to provide an amino moiety

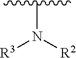

of formula (ii-a):

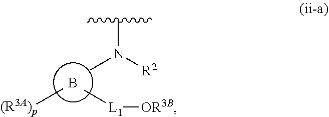

wherein Ring B is the C₃-C₁₀ carbocyclyl or 4- to 10-membered heterocyclyl, and p is 0, 1, 2, or 3.

In some embodiments, Ring B is a C₃-C₁₀ carbocyclyl or 4- to 10-membered heterocyclyl.

In some embodiments, Ring B is a C₃-C₁₀ carbocyclyl. In some embodiments, Ring B is a monocyclic C₃-C₈ carbocyclyl. In some embodiments, Ring B is a monocyclic C₅-C₇ carbocyclyl. In some embodiments, Ring B is a bicyclic C₅-C₈ carbocyclyl. In some embodiments, Ring B is a bicyclic C₉-C₁₀ carbocyclyl. In some embodiments, Ring B is 4- to 10-membered heterocyclyl. In some embodiments, Ring B is monocyclic 4- to 8-membered heterocyclyl. In some embodiments, Ring B is monocyclic 4- to 6-membered heterocyclyl. In some embodiments, Ring B is monocyclic 5- to 6-membered heterocyclyl.

In some embodiments, amino moieties

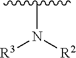

which fall within the scope of formula (ii-a) include, but are not limited to:

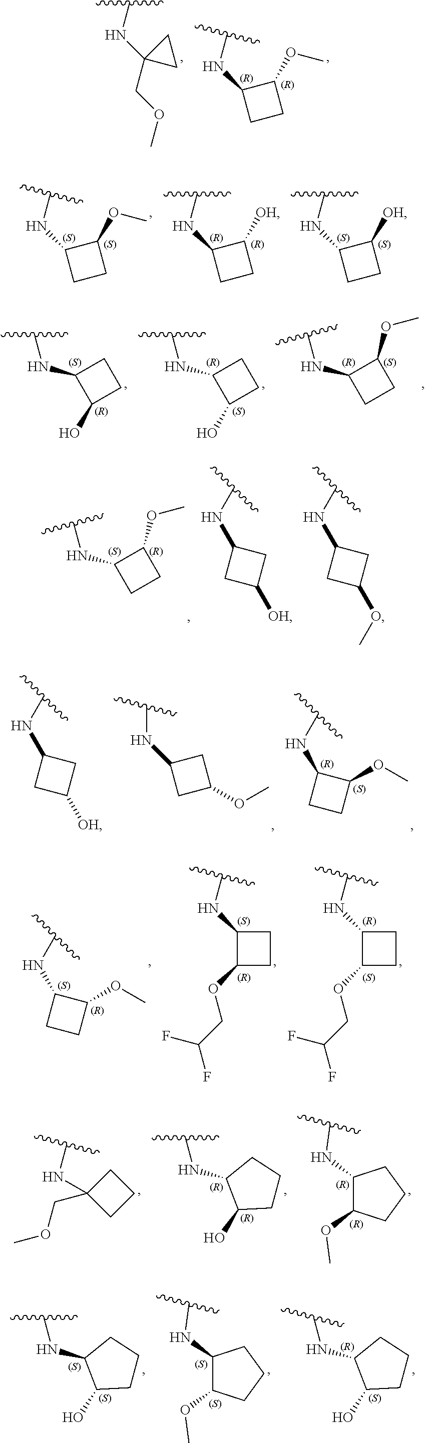

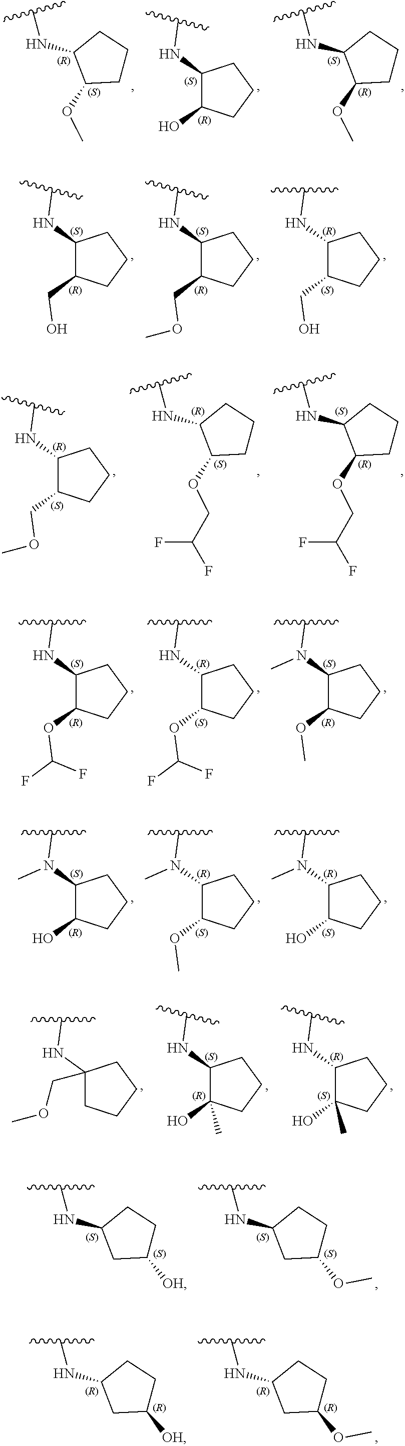

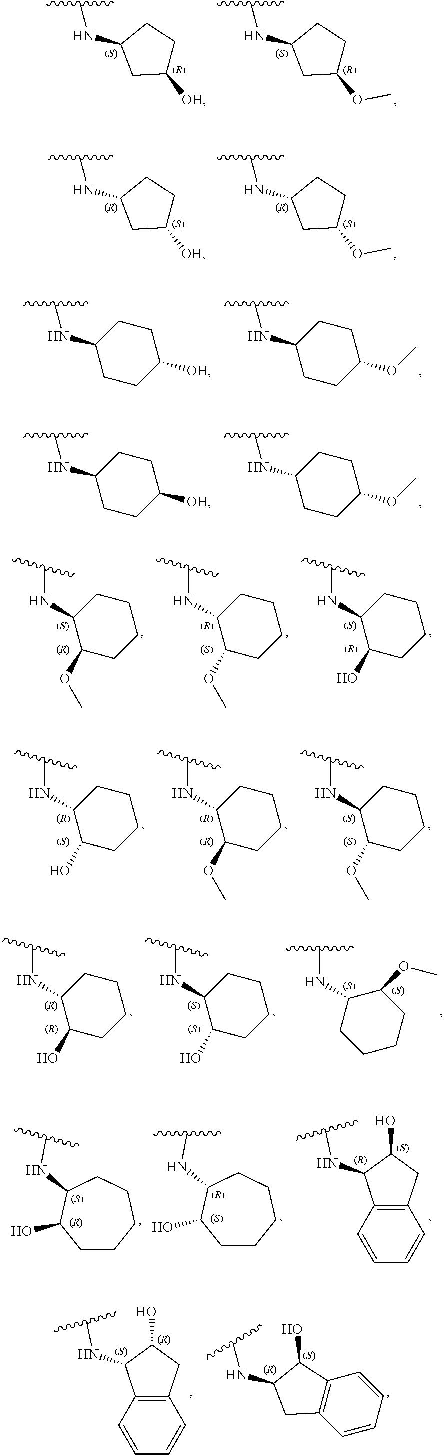

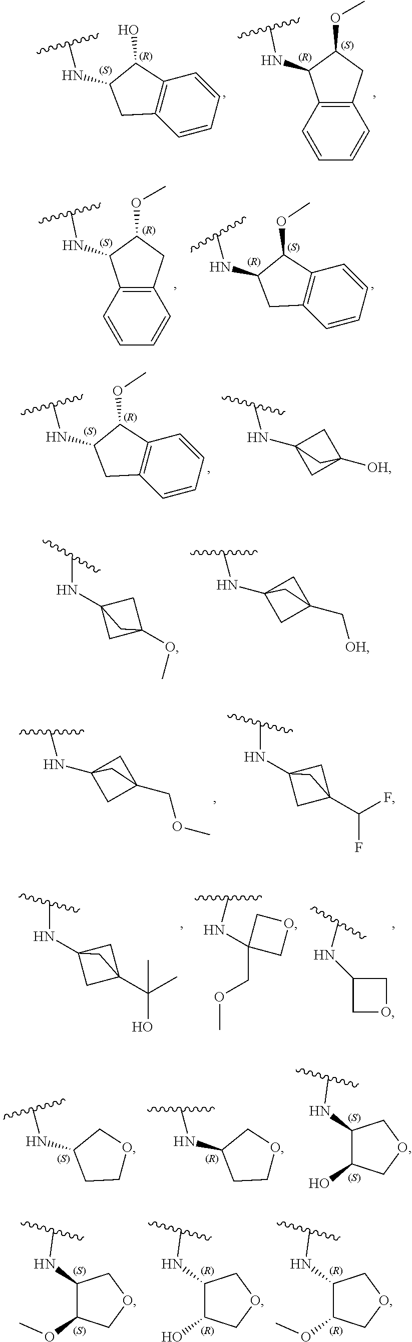

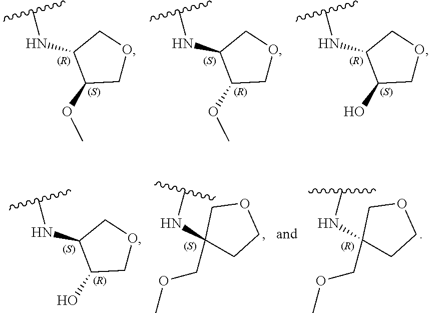

In some embodiments, amino moieties

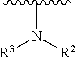

which fall within the scope of formula (ii-a) include, but are not limited to:

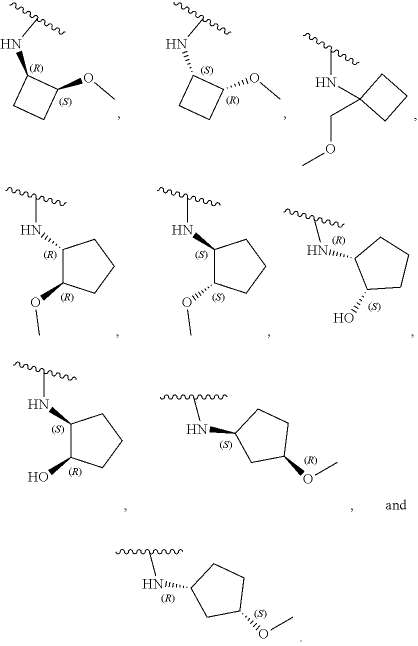

In some embodiments, R² and R³ are joined, with the atom to which they are attached, to form a 4- to 10-membered heterocyclyl independently substituted with 0, 1, 2, 3, or 4 R^{3A}.

In some embodiments, R² and R³ are joined, with the atom to which they are attached, to form a 4-membered heterocyclyl substituted with 0, 1, 2, 3, or 4 R^{3A}.

In some embodiments, R² and R³ are joined, with the atom to which they are attached, to form a 4-membered heterocyclyl.

In some embodiments, R² and R³ are joined, with the atom to which they are attached, to form a 4-membered heterocyclyl substituted with 1 R^{3A}.

In some embodiments, R² and R³ are joined, with the atom to which they are attached, to form a 4-membered heterocyclyl substituted with 2 R^{3A}.

In some embodiments, R² and R³ are joined, with the atom to which they are attached, to form a 4-membered heterocyclyl substituted with 3 R^{3A}.

In some embodiments, R² and R³ are joined, with the atom to which they are attached, to form a 5-membered heterocyclyl.

In some embodiments, R² and R³ are joined, with the atom to which they are attached, to form a 5-membered heterocyclyl substituted with 1 R^{3A}.

In some embodiments, R² and R³ are joined, with the atom to which they are attached, to form a 5-membered heterocyclyl substituted with 2 R^{3A}.

In some embodiments, R² and R³ are joined, with the atom to which they are attached, to form a 5-membered heterocyclyl substituted with 3 R^{3A}.

In some embodiments, R² and R³ are joined, with the atom to which they are attached, to form a 6-membered heterocyclyl.

In some embodiments, R² and R³ are joined, with the atom to which they are attached, to form a 6-membered heterocyclyl substituted with 1 R^{3A}.

In some embodiments, R² and R³ are joined, with the atom to which they are attached, to form a 6-membered heterocyclyl substituted with 2 R^{3A}.

In some embodiments, R² and R³ are joined, with the atom to which they are attached, to form a 6-membered heterocyclyl substituted with 3 R^{3A}.

In some embodiments, the amino moiety

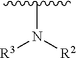

is selected from the group consisting of:

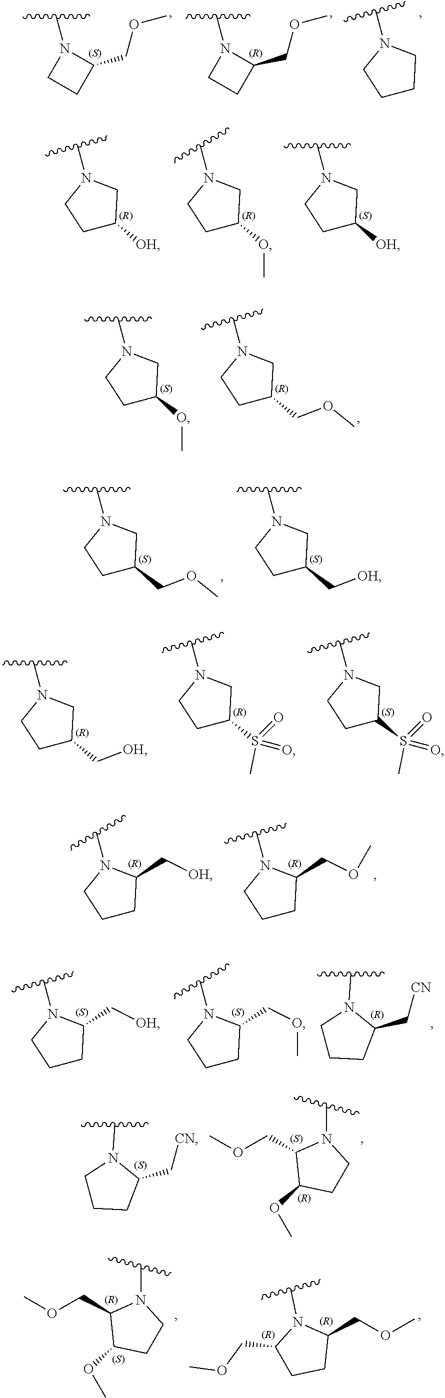

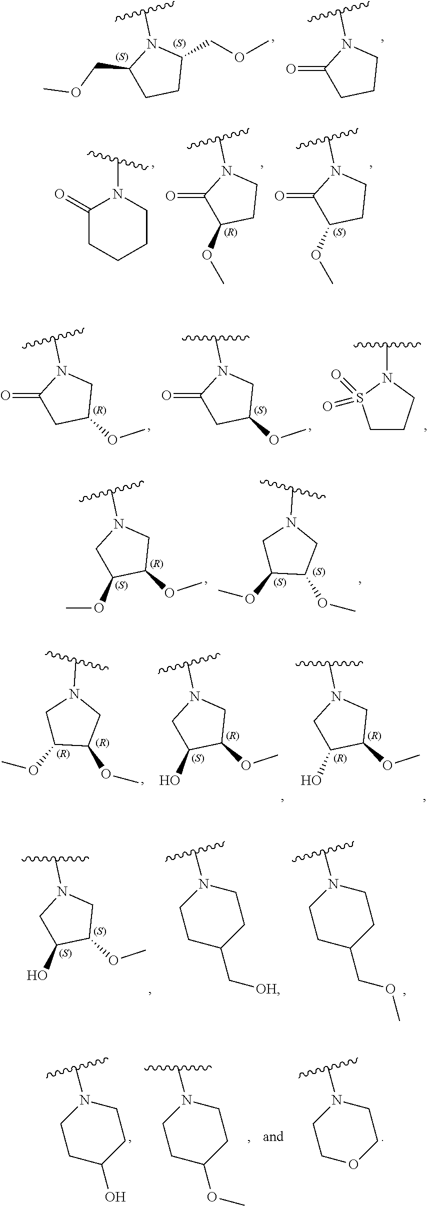

In some embodiments, the amino moiety

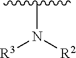

is selected from the group consisting of:

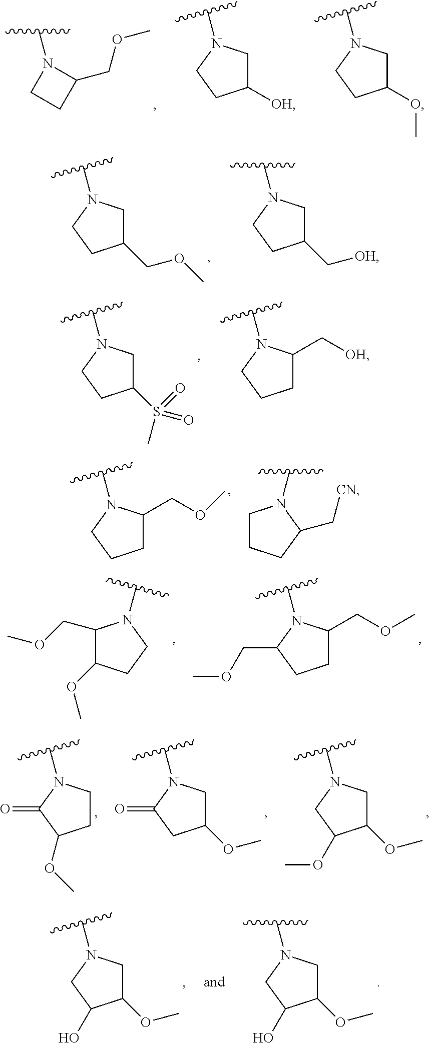

In some embodiments, R² and R³ of the amino moiety of formula

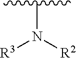

are joined to form an amino moiety of formula

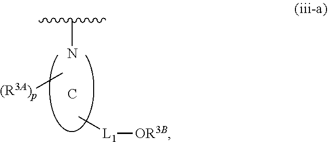

wherein L₁ is a bond, C₁-C₃ alkylene, or C₁-C₃ haloalkylene, Ring C is a 5- to 10-membered heterocyclyl, and p is 0, 1, 2, or 3.

In some embodiments, amino moieties

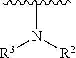

which fall within the scope of formula (iii-a) include, but are not limited to:

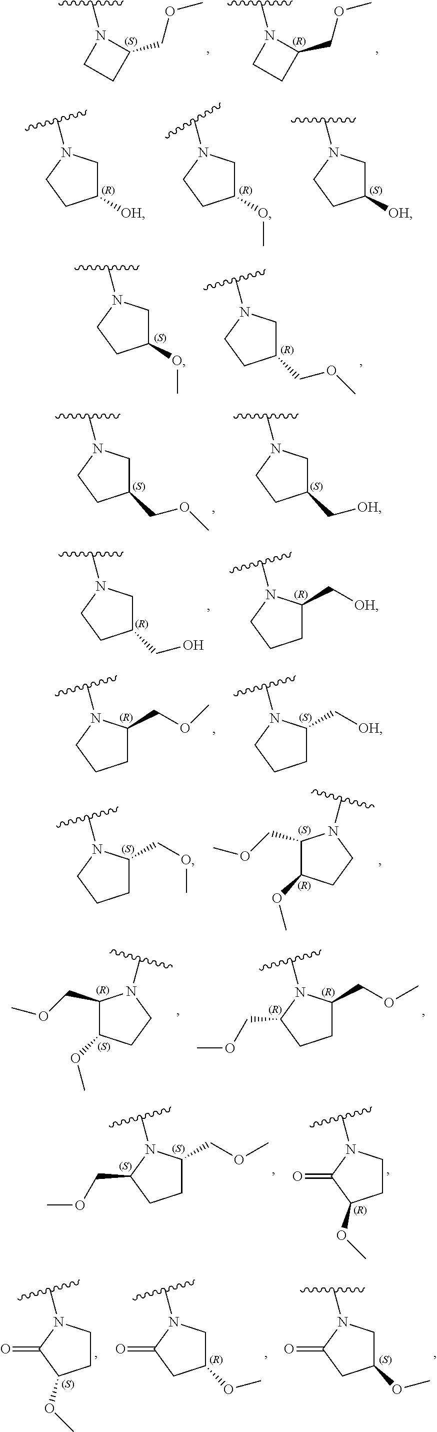

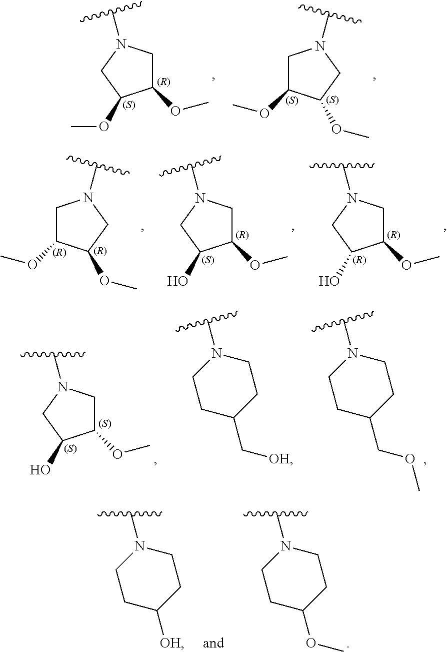

As generally defined herein, each R^{3A} is independently C₁-C₃ alkyl, C₂-C₃ alkenyl, C₂-C₃ alkynyl, halogen. ═O, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D}.

In some embodiments, each R^{3A} is independently C₁-C₃ alkyl, C₂-C₃ alkenyl, C₂-C₃ alkynyl, halogen. ═O, -L₁-CN, -L₁-SOR^{3C}, -L₁-SO₂R^{3C}, -L₁-S₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D}.

In some embodiments, each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), wherein the alkyl, alkenyl, alkynyl, carbocyclyl, heterocyclyl, aryl, and heteroaryl are independently substituted with 0, 1, 2, 3, or 4 R^{3D}.

In some embodiments, each R^{3A} is independently C₁-C₃ alkyl, halogen, ═O, -L₁-CN, -L₁-SO₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), wherein the alkyl, carbocyclyl, heterocyclyl, aryl, and heteroaryl are independently substituted with 0, 1, 2, 3, or 4 R^{3D}.

In some embodiments, at least one R^{3A} is independently C₁-C₃ alkyl substituted with 0, 1, 2, 3, or 4 R^{3D}.

In some embodiments, at least one R^{3A} is independently C₁-C₃ alkyl.

In some embodiments, at least one R^{3A} is independently C₁-C₃ alkyl substituted with 1 R^{3D}.

In some embodiments, at least one R^{3A} is independently C₁-C₃ alkyl substituted with 2 R^{3D}. In some embodiments, at least one R^{1A} is independently C₁-C₃ alkyl substituted with 3 R^{3D}. In some embodiments, at least one R^{3A} is independently C₁-C₃ alkyl substituted with 4 R^{3D}.

In some embodiments, at least one R^{3A} is independently halogen or ═O.

In some embodiments, at least one R^{3A} is independently halogen.

In some embodiments, at least one R^{3A} is independently F or Cl.

In some embodiments, at least one R^{3A} is independently F. In some embodiments, at least one R^{3A} is independently Cl.

In some embodiments, at least one R^{3A} is independently ═O.

In some embodiments, at least one R^{3A} is independently -L₁-CN, -L₁-SO₂R^{3C}, -L₁-OR^{3B}, or -L₁-N(R^{3B})₂.

In some embodiments, at least one R^{3A} is independently -L₁-CN.

In some embodiments, at least one R^{3A} is independently —CN.

In some embodiments, at least one R^{3A} is independently —(C₁-C₃ alkylene)-CN.

In some embodiments, at least one R^{3A} is independently -L₁-SO₂R^{3C}.

In some embodiments, at least one R^{3A} is independently —SO₂R³⁷.

In some embodiments, at least one R^{3A} is independently —(C₁-C₃ alkylene)-SO₂R^{3C}.

In some embodiments, at least one R^{3A} is independently -L₁-PO(R^{3C})₂.

In some embodiments, at least one R^{3A} is independently —PO(R^{3C})₂.

In some embodiments, at least one R^{3A} is independently -L₁-OR^{3B}.

In some embodiments, at least one R^{3A} is independently —OR^{3B}.

In some embodiments, at least one R^{3A} is independently -L₁-C(═O)N(R^{3B})₂ or -L₁-C(═O)OR^{3B}.

In some embodiments, at least one R^{3A} is independently —C(═O)N(R^{3B})₂ or —C(═O)OR^{3B}.

In some embodiments, at least one R^{3A} is independently —(C₁-C₃ alkylene)-OR^{3B}.

In some embodiments, at least one R^{3A} is independently -L₁-(C₃-C₆ carbocyclyl), -L₁-(4- to 6-membered heterocyclyl), -L₁-(C₆₋₁₀ aryl), or -L₁-(5- to 10-membered heteroaryl), wherein the carbocyclyl, heterocyclyl, aryl, and heteroaryl are independently substituted with 0, 1, 2, 3, or 4 R^{3D}.

In some embodiments, at least one R^{3A} is independently -L₁-(C₃-C₆ carbocyclyl) substituted with 0, 1, 2, 3, or 4 R^{3D}.

In some embodiments, at least one R^{3A} is independently -L₁-(C₃-C₆ carbocyclyl).

In some embodiments, at least one R^{3A} is independently -L₁-(C₃-C₆ carbocyclyl) substituted with 1 R^{3D}.

In some embodiments, at least one R^{3A} is independently -L₁-(C₃-C₆ carbocyclyl) substituted with 2 R^{3D}. In some embodiments, at least one R^{3A} is independently -L₁-(C₃-C₆ carbocyclyl) substituted with 3 R^{3D}. In some embodiments, at least one R^{3A} is independently -L₁-(C₃-C₆ carbocyclyl) substituted with 4 R^{3D}.

In some embodiments, at least one R^{3A} is independently —(C₃-C₆ carbocyclyl) substituted with 0, 1, 2, 3, or 4 R^{3D}.

In some embodiments, at least one R^{3A} is independently —(C₃-C₆ carbocyclyl).

In some embodiments, at least one R^{3A} is independently —(C₃-C₆ carbocyclyl) substituted with 1 R^{3D}.

In some embodiments, at least one R^{3A} is independently —(C₃-C₆ carbocyclyl) substituted with 2 R^{3D}. In some embodiments, at least one R^{3A} is independently —(C₃-C₆ carbocyclyl) substituted with 3 R^{3D}. In some embodiments, at least one R^{3A} is independently —(C₃-C₆ carbocyclyl) substituted with 4 R^{3D}.

In some embodiments, at least one R^{3A} is independently —(C₁-C₃ alkylene)-(C₃-C₆ carbocyclyl) substituted with 0, 1, 2, 3, or 4 R^{3D}.

In some embodiments, at least one R^{3A} is independently —(C₁-C₃ alkylene)-(C₃-C₆ carbocyclyl).

In some embodiments, at least one R^{3A} is independently —(C₁-C₃ alkylene)-(C₃-C₆ carbocyclyl) substituted with 1 R^{3D}.

In some embodiments, at least one R^{3A} is independently —(C₁-C₃ alkylene)-(C₃-C₆ carbocyclyl) substituted with 2 R³¹. In some embodiments, at least one R^{3A} is independently —(C₁-C₃ alkylene)-(C₁-C₆ carbocyclyl) substituted with 3 R^{3D}. In some embodiments, at least one R^{3A} is independently —(C₁-C₃ alkylene)-(C₃-C₆ carbocyclyl) substituted with 4 R^{3D}.

In some embodiments, at least one R^{3A} is independently -L₁-(4- to 6-membered heterocyclyl) substituted with 0, 1, 2, 3, or 4 R^{3D}.

In some embodiments, at least one R^{3A} is independently -L₁-(4- to 6-membered heterocyclyl).

In some embodiments, at least one R^{3A} is independently -L₁-(4- to 6-membered heterocyclyl) substituted with 1 R^{3D}.

In some embodiments, at least one R^{3A} is independently -Le-(4- to 6-membered heterocyclyl) substituted with 2 R^{3D}. In some embodiments, at least one R^{3A} is independently -L₁-(4- to 6-membered heterocyclyl) substituted with 3 R^{3D}. In some embodiments, at least one R^{3A} is independently -L₁-(4- to 6-membered heterocyclyl) substituted with 4 R^{3D}.

In some embodiments, at least one R^{3A} is independently -(4- to 6-membered heterocyclyl) substituted with 0, 1, 2, 3, or 4 R^{3D}.

In some embodiments, at least one R^{3A} is independently -(4- to 6-membered heterocyclyl).

In some embodiments, at least one R^{3A} is independently -(4- to 6-membered heterocyclyl) substituted with 1 R^{3D}.

In some embodiments, at least one R^{3A} is independently -(4- to 6-membered heterocyclyl) substituted with 2 R^{3D}. In some embodiments, at least one R^{3A} is independently -(4- to 6-membered heterocyclyl) substituted with 3 R^{3D}. In some embodiments, at least one R^{3A} is independently -(4- to 6-membered heterocyclyl) substituted with 4 R^{3D}.

In some embodiments, at least one h R^{3A} is independently —(C₁-C₃ alkylene)-(4- to 6-membered heterocyclyl) substituted with 0, 1, 2, 3, or 4 R^{3D}.

In some embodiments, at least one R^{3A} is independently —(C₁-C₃ alkylene)-(4- to 6-membered heterocyclyl).

In some embodiments, at least one R^{3A} is independently —(C₁-C₃ alkylene)-(4- to 6-membered heterocyclyl) substituted with 1 R^{3D}.

In some embodiments, at least one R^{3A} is independently —(C₁-C₃ alkylene)-(4- to 6-membered heterocyclyl) substituted with 2 R^{3D}. In some embodiments, at least one R^{3A} is independently —(C₁-C₃ alkylene)-(4- to 6-membered heterocyclyl) substituted with 3 R^{3D}. In some embodiments, at least one R^{3A} is independently —(C₁-C₃ alkylene)-(4- to 6-membered heterocyclyl) substituted with 4 R^{3D}.

In some embodiments, at least one R^{3A} is independently -L₁-(C₆₋₁₀ aryl) substituted with 0, 1, 2, 3, or 4 R^{3D}.

In some embodiments, at least one R^{3A} is independently -L₁-(C₆ aryl) substituted with 0, 1, 2, 3, or 4 R^{3D}.

In some embodiments, at least one R^{3A} is independently -L₁-(C₆ aryl).

In some embodiments, at least one R^{3A} is independently -L₁-(C₆ aryl) substituted with 1 R^{3D}.

In some embodiments, at least one R^{3A} is independently -L₁-(C₆ aryl) substituted with 2 R³¹. In some embodiments, at least one R^{3A} is independently -L₁-(C₆ aryl) substituted with 3 R^{3D}. In some embodiments, at least one R^{3A} is independently -L₁-(C₆ aryl) substituted with 4 R^{3D}.

In some embodiments, at least one R^{3A} is independently —(C₆ aryl) substituted with 0, 1, 2, 3, or 4 R^{3D}.

In some embodiments, at least one R^{3A} is independently —(C₆ aryl).

In some embodiments, at least one R^{3A} is independently —(C₆ aryl) substituted with 1 R^{3D}.

In some embodiments, at least one R^{3A} is independently —(C₆ aryl) substituted with 2 R^{3D}. In some embodiments, at least one R^{3A} is independently —(C₆ aryl) substituted with 3 R^{3D}. In some embodiments, at least one R^{3A} is independently —(C₆ aryl) substituted with 4 R^{3D}.

In some embodiments, at least one R^{3A} is independently —(C₁-C₃ alkylene)-(C₆ aryl) substituted with 0, 1, 2, 3, or 4 R^{3D}.

In some embodiments, at least one R^{3A} is independently —(C₁-C₃ alkylene)-(C₆ aryl).

In some embodiments, at least one R^{3A} is independently —(C₁-C₃ alkylene)-(C₆ aryl) substituted with 1 R^{3D}.

In some embodiments, at least one R^{3A} is independently —(C₁-C₃ alkylene)-(C₆ aryl) substituted with 2 R^{3D}. In some embodiments, at least one R^{3A} is independently —(C₁-C₃ alkylene)-(C₆ aryl) substituted with 3 R^{3D}. In some embodiments, at least one R^{3A} is independently —(C₁-C₃ alkylene)-(C₆ aryl) substituted with 4 R^{3D}.

In some embodiments, at least one R^{3A} is independently -L₁-(5- to 10-membered heteroaryl) substituted with 0, 1, 2, 3, or 4 R^{3D}.

In some embodiments, at least one R^{3A} is independently -L₁-(5- to 10-membered heteroaryl).

In some embodiments, at least one R^{3A} is independently -L₁-(5- to 10-membered heteroaryl) substituted with 1 R^{3D}.

In some embodiments, at least one R^{3A} is independently -L₁-(5- to 10-membered heteroaryl) substituted with 2 R^{3D}. In some embodiments, at least one R^{3A} is independently -L₁-(5- to 10-membered heteroaryl) substituted with 3 R^{3D}. In some embodiments, at least one R^{3A} is independently -L₁-(5- to 10-membered heteroaryl) substituted with 4 R^{3D}.

In some embodiments, at least one R^{3A} is independently -(5- to 10-membered heteroaryl) substituted with 0, 1, 2, 3, or 4 R^{3D}.

In some embodiments, at least one R^{3A} is independently -(5- to 10-membered heteroaryl).

In some embodiments, at least one R^{3A} is independently -(5- to 10-membered heteroaryl) substituted with 1 R^{3D}.

In some embodiments, at least one R^{3A} is independently -(5- to 10-membered heteroaryl) substituted with 2 R^{3D}. In some embodiments, each R^{3A} is independently -(5- to 10-membered heteroaryl) substituted with 3 R^{3D}. In some embodiments, at least one R^{3A} is independently -(5- to 10-membered heteroaryl) substituted with 4 R^{3D}.

In some embodiments, at least one R^{3A} is independently —(C₁-C₃ alkylene)-(5- to 10-membered heteroaryl) substituted with 0, 1, 2, 3, or 4 R^{3D}.

In some embodiments, at least one R^{3A} is independently —(C₁-C₃ alkylene)-(5- to 10-membered heteroaryl).

In some embodiments, at least one R^{3A} is independently —(C₁-C₃ alkylene)-(5- to 10-membered heteroaryl) substituted with 1 R^{3D}.

In some embodiments, at least one R^{3A} is independently —(C₁-C₃ alkylene)-(5- to 10-membered heteroaryl) substituted with 2 R^{3D}. In some embodiments, at least one R^{3A} is independently —(C₁-C₃ alkylene)-(5- to 10-membered heteroaryl) substituted with 3 R^{3D}. In some embodiments, at least one R^{3A} is independently —(C₁-C₃ alkylene)-(5- to 10-membered heteroaryl) substituted with 4 R^{3D}.

In some embodiments, two R^{3A} groups are joined, with the atoms to which they are attached, to form C₆ aryl, 5- to 6-membered heteroaryl, C₃-C₆ carbocyclyl, or 4- to 6-membered heterocyclyl.

In some embodiments, two R^{3A} groups are joined, with the atoms to which they are attached, to form C₆ aryl.

In some embodiments, two R^{3A} groups are joined, with the atoms to which they are attached, to form 5- to 6-membered heteroaryl.

In some embodiments, two R^{3A} groups are joined, with the atoms to which they are attached, to form C₁-C₆ carbocyclyl.

In some embodiments, two R^{3A} groups are joined, with the atoms to which they are attached, to form 4- to 6-membered heterocyclyl.

As generally defined herein, each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D}.

In some embodiments, at least one R^{3B} is independently hydrogen.

In some embodiments, each R^{3B} is independently C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D}.

In some embodiments, each R^{3B} is independently C₁-C₃ alkyl, C₃-C₆ carbocyclyl, or 4- to 6-membered heterocyclyl.

In some embodiments, at least one R^{3B} is independently C₁-C₃ alkyl substituted with 0, 1, 2, 3, or 4 R^{3D}.

In some embodiments, at least one R^{3B} is independently C₁-C₃ alkyl.

In some embodiments, at least one R^{3B} is independently C₁-C₃ alkyl substituted with 1 R^{3D}.

In some embodiments, at least one R^{3B} is independently C₁-C₃ alkyl substituted with 2 R^{3D}.

In some embodiments, at least one R^{3B} is independently C₁-C₃ alkyl substituted with 3 R^{3D}.

In some embodiments, at least one R^{3B} is independently C₁-C₃ alkyl substituted with 4 R^{3D}.

In some embodiments, at least one R^{3B} is independently C₃-C₆ carbocyclyl substituted with 0, 1, 2, 3, or 4 R^{3D}.

In some embodiments, at least one R^{3B} is independently C₃-C₆ carbocyclyl.

In some embodiments, at least one R^{3B} is independently C₃-C₆ carbocyclyl substituted with 1 R^{3D}.

In some embodiments, at least one R^{3B} is independently C₃-C₆ carbocyclyl substituted with 2 R^{3D}.

In some embodiments, at least one R^{3B} is independently C₃-C₆ carbocyclyl substituted with 3 R^{3D}.

In some embodiments, at least one R^{3B} is independently C₃-C₆ carbocyclyl substituted with 4 R^{3D}.

In some embodiments, at least one R^{3B} is independently 4- to 6-membered heterocyclyl substituted with 0, 1, 2, 3, or 4 R^{3D}.

In some embodiments, at least one R^{3B} is independently 4- to 6-membered heterocyclyl.

In some embodiments, at least one R^{3B} is independently 4- to 6-membered heterocyclyl substituted with 1 R^{3D}.

In some embodiments, at least one R^{3B} is independently 4- to 6-membered heterocyclyl substituted with 2 R^{3D}.

In some embodiments, at least one R^{3B} is independently 4- to 6-membered heterocyclyl substituted with 3 R^{3D}.

In some embodiments, at least one R^{3B} is independently 4- to 6-membered heterocyclyl substituted with 4 R^{3D}.

As generally defined herein, each R^{3C} is independently C₁-C₃ alkyl or C₁-C₃ haloalkyl.

In some embodiments, at least one R^{3C} is independently C₁-C₃ alkyl.

In some embodiments, at least one R^{3C} is independently C₁-C₃ haloalkyl.

As generally defined herein, each R^{3D} is independently halogen, —OR^{3E}, C₁-C₃ alkyl, or C₁-C₃ haloalkyl.

In another aspect, each R^{3D} is independently halogen, —OR^{3E}, —CN, C₁-C₃ alkyl, or C₁-C₃ haloalkyl.

In some embodiments, each R^{3D} is independently halogen or —OC₁-C₃ alkyl.

In some embodiments, at least one R^{3D} is independently halogen.

In some embodiments, at least one R^{3D} is independently F or Cl.

In some embodiments, at least one R^{3D} is independently F. In some embodiments, at least one R^{3D} is independently Cl.

In some embodiments, at least one R^{3D} is independently —OR^{3E}.

In some embodiments, at least one R^{3D} is independently —OC₁-C₃ alkyl.

In some embodiments, at least one R^{3D} is —CN.

As generally defined herein, each R^{3E} is independently hydrogen, C₁-C₄ alkyl, or C₁-C₄ haloalkyl.

In some embodiments, each R^{3E} is independently hydrogen, C₁-C₃ alkyl, or C₁-C₃ haloalkyl.

In some embodiments, at least one R^{3E} is independently hydrogen.

In some embodiments, at least one R^{3E} is independently C₁-C₃ alkyl.

In some embodiments, at least one R^{3E} is independently C₁-C₃ haloalkyl.

As generally defined herein, each L₁ is independently a bond, C₁-C₃ alkylene, or C₁-C₃ haloalkylene.

In some embodiments, each L₁ is independently a bond or C₁-C₃ alkylene.

In some embodiments, at least one L₁ is independently a bond.

In some embodiments, at least one L₁ is independently C₁-C₃ alkylene.

In some embodiments, at least one L₁ is independently branched C₁-C₃ alkylene.

In some embodiments, at least one L₁ is independently C₁ alkylene. In some embodiments, at least one L₁ is independently C₂ alkylene. In some embodiments, at least one L₁ is independently C₃ alkylene.

(b) Ring A, R⁴, L₂, and m

As generally defined herein, Ring A is a 5-membered monocyclic heteroaryl.

In some embodiments, Ring A is a 5-membered monocyclic heteroaryl comprising 1 or 2 ring heteroatoms independently selected from nitrogen, oxygen, and sulfur.

In some embodiments, Ring A is a 5-membered monocyclic heteroaryl comprising 1 ring nitrogen atom.

In some embodiments, Ring A is a 5-membered monocyclic heteroaryl comprising 2 ring nitrogen atoms.

In some embodiments, Ring A is a 5-membered monocyclic heteroaryl comprising 1 ring oxygen atom.

In some embodiments, Ring A is a 5-membered monocyclic heteroaryl comprising 1 ring nitrogen atom and 1 ring oxygen atom.

In some embodiments, Ring A is a 5-membered monocyclic heteroaryl comprising 1 ring sulfur atom.

In some embodiments, Ring A is a 5-membered monocyclic heteroaryl comprising 1 ring nitrogen atom and 1 ring sulfur atom.

In some embodiments, Ring A is a pyrrole, furan, thiophene, pyrazole, imidazole, isoxazole, oxazole, isothiazole, or thiazole ring.

In some embodiments, Ring A is:

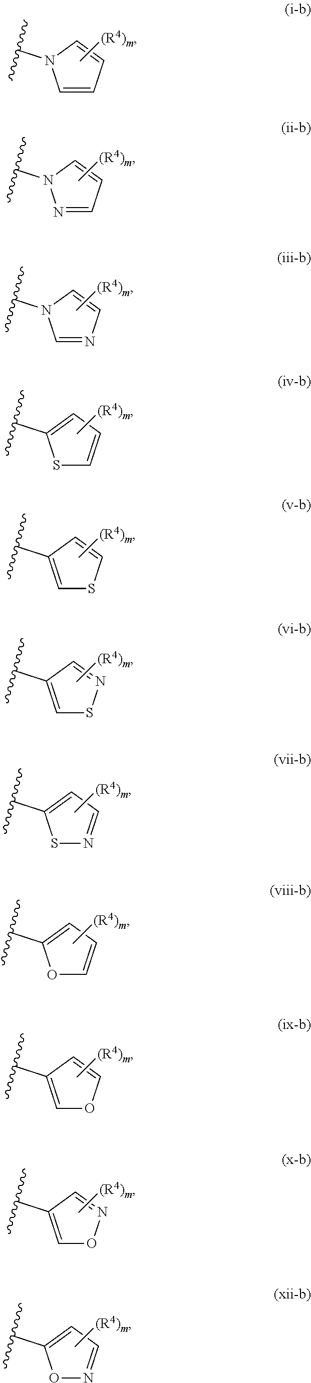

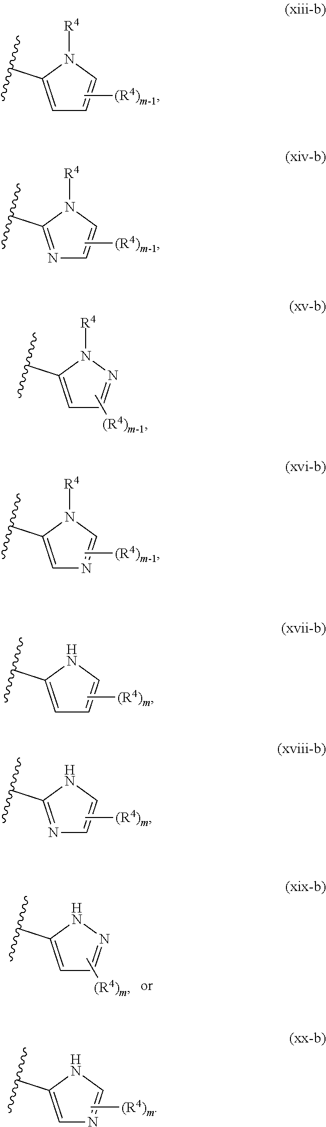

In some embodiments, Ring A is:

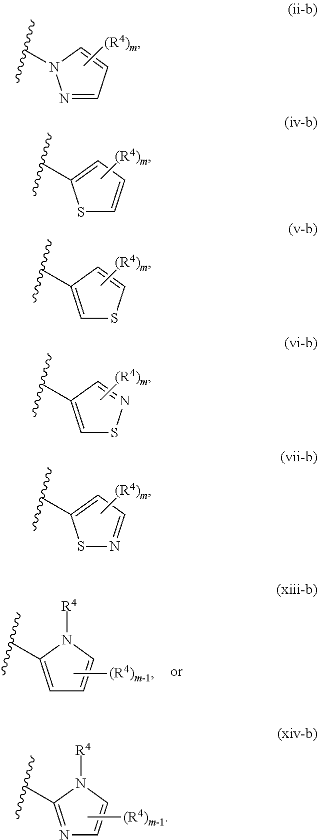

In some embodiments, Ring A is:

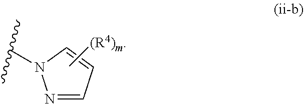

In some embodiments, Ring A is:

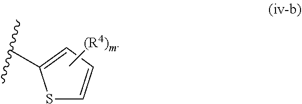

In some embodiments, Ring A is:

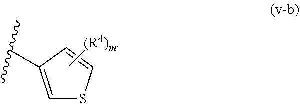

In some embodiments, Ring A is:

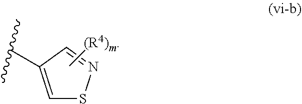

In some embodiments, Ring A is:

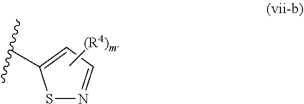

In some embodiments, Ring A is:

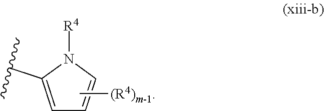

In some embodiments, Ring A is:

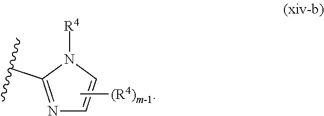

In some embodiments, Ring A is a 5-membered monocyclic heteroaryl directly linked to the thiadiazole via an N atom, as provided in formula (xvii-b):

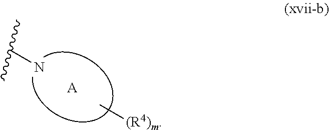

Exemplary Ring A ring systems that fall within the scope of formula (xvii-b) include, but are not limited to:

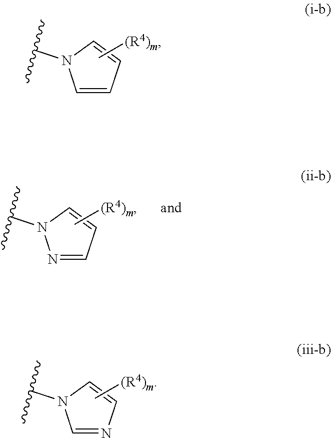

As generally defined herein, each R⁴ is independently halogen, —CN, -L₂-OR^{4A}, -L₂-N(R^{4B})₂, C₁-C₆ alkyl, or C₁-C₆ haloalkyl, wherein R^{4A} and R^{4B} are each independently hydrogen, C₁₋₃alkyl, C₁₋₃ haloalkyl, —C(═O)R^{4C}, wherein R^{4C} is C₁-C₆ alkyl or C₁-C₆ haloalkyl; each L₂ is a bond, C₁-C₃ alkylene, or C₁-C₃ haloalkylene; and m is 0, 1 or 2.

As generally defined herein, each R⁴ is independently halogen, —CN, -L₂-OR^{4A}, -L₂-N(R^{4B})₂, C₁-C₆ alkyl, or C₁-C₆ haloalkyl, wherein R^{4A} and R^{4B} are each independently hydrogen, C₁₋₃ alkyl, or C₁₋₃ haloalkyl; each L₂ is a bond, C₁-C₃ alkylene, or C₁-C₃ haloalkylene; and m is 0, 1 or 2.

In some embodiments, each R⁴ is independently halogen, —CN, -L₂-OR^{4A}, -L₂-N(R^{4B})₂, C₁-C₆ alkyl, or C₁-C₆ haloalkyl.

In some embodiments, at least one R⁴ is independently halogen.

In some embodiments, at least one R⁴ is independently —F or —Cl.

In some embodiments, at least one R⁴ is independently —F. In some embodiments, at least one R⁴ is independently —Cl.

In some embodiments, at least one R⁴ is independently —CN.

In some embodiments, at least one R⁴ is independently -L₂-OR^{4A}.

In some embodiments, at least one R⁴ is independently —OR^{4A}.

In some embodiments, at least one R⁴ is independently —(C₁-C₃ alkylene)-OR^{4A}.

In some embodiments, at least one R⁴ is independently —(C₁ alkylene)-OR^{4A}.

In some embodiments, at least one R⁴ is independently —(C₂ alkylene)-OR^{4A}.

In some embodiments, at least one R⁴ is independently —(C₃ alkylene)-OR^{4A}.

In some embodiments, at least one R⁴ is independently —OH.

In some embodiments, at least one R⁴ is independently —(C₁-C₃ alkylene)-OH.

In some embodiments, at least one R⁴ is independently —(C₁ alkylene)-OH.

In some embodiments, at least one R⁴ is independently —(C₂ alkylene)-OH.

In some embodiments, at least one R⁴ is independently —(C₃ alkylene)-OH.

In some embodiments, at least one R⁴ is independently —O(C₁-C₃ alkyl).

In some embodiments, at least one R⁴ is independently —(C₁-C₃ alkylene)-O(C₁-C₃ alkyl).

In some embodiments, at least one R⁴ is independently —(C₁ alkylene)-O(C₁-C₃ alkyl).

In some embodiments, at least one R⁴ is independently —(C₂ alkylene)-O(C₁-C₃ alkyl).

In some embodiments, at least one R⁴ is independently —(C₃ alkylene)-O(C₁-C₃ alkyl).

In some embodiments, at least one R⁴ is independently —N(R^{4B})₂.

In some embodiments, at least one R⁴ is independently -L₂-N(R^{4B})₂.

In some embodiments, at least one R⁴ is independently —(C₁-C₃ alkylene)-N(R^{4B})₂.

In some embodiments, at least one R⁴ is independently —NH₂.

In some embodiments, at least one R⁴ is independently -L₂-NH₂.

In some embodiments, at least one R⁴ is independently —(C₁-C₃ alkylene)-NH₂.

In some embodiments, at least one R⁴ is independently —NH(R^{4B}).

In some embodiments, at least one R⁴ is independently -L₂-NH(R^{4B}).

In some embodiments, at least one R⁴ is independently —(C₁-C₃ alkylene)-NH(R^{4B}).

In some embodiments, at least one R⁴ is independently —N(C₁-C₃ alkyl)₂.

In some embodiments, at least one R⁴ is independently -L₂-N(C₁-C₃ alkyl)₂.

In some embodiments, at least one R⁴ is independently —(C₁-C₃ alkylene)-N(C₁-C₃ alkyl)₂.

In some embodiments, at least one R⁴ is independently C₁-C₆ alkyl.

In some embodiments, at least one R⁴ is independently methyl. In some embodiments, at least one R⁴ is independently ethyl. In some embodiments, at least one R⁴ is independently propyl. In some embodiments, at least one R⁴ is independently isopropyl. In some embodiments, at least one R⁴ is independently butyl. In some embodiments, at least one R⁴ is independently isobutyl. In some embodiments, at least one R⁴ is independently tert-butyl.

In some embodiments, at least one R⁴ is independently C₁-C₆ haloalkyl.

In some embodiments, at least one R⁴ is independently halomethyl. In some embodiments, at least one R⁴ is independently haloethyl. In some embodiments, at least one R⁴ is independently halopropyl. In some embodiments, at least one R⁴ is independently halo-isopropyl. In some embodiments, at least one R⁴ is independently halobutyl. In some embodiments, at least one R⁴ is independently halo-isobutyl. In some embodiments, at least one R⁴ is independently halo-tert-butyl.

In some embodiments, each instance of R⁴ is independently selected from the group consisting of —CH₃, —CH₂CH₃, —CHF₂, —CF₃, —Cl, —CN, —NH₂, and —CH₂OH.

In some embodiments, at least one instance of R⁴ is independently —CH₃ or —CH₂CH₃.

In some embodiments, at least one instance of R⁴ is independently —CHF₂ or —CF₃.

In some embodiments, Ring A is selected from the group consisting of:

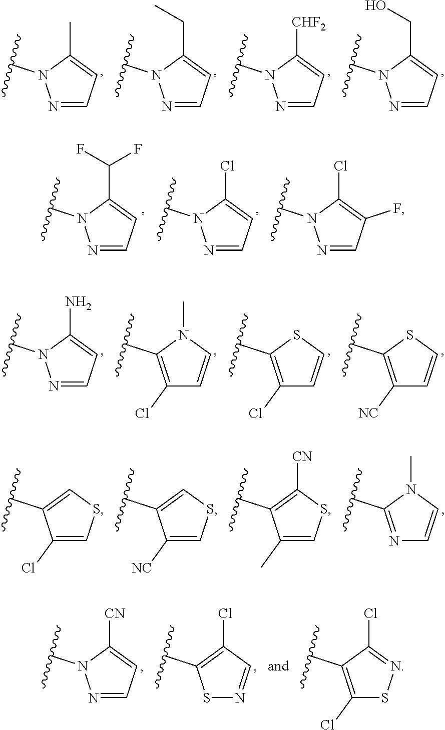

In some embodiments, Ring A ring systems of formula (xvii-b) are selected from the group consisting of:

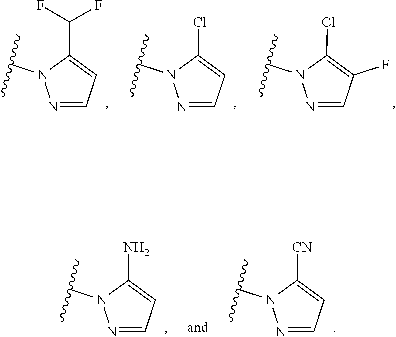

In some embodiments, Ring A is:

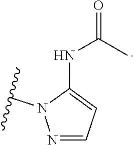 [SMILES: *n1nccc1NC(C)=O]

As generally defined herein, each L₂ is independently a bond, C₁-C₃ alkylene, or C₁-C₃ haloalkylene.

In some embodiments, each L₂ is independently a bond or C₁-C₃ alkylene.

In some embodiments, at least one L₂ is independently a bond.

In some embodiments, at least one L₁ is independently C₁-C₃ alkylene.

In some embodiments, at least one L₂ is independently C₁ alkylene. In some embodiments, at least one L₂ is independently C₂ alkylene. In some embodiments, at least one L₂ is independently C₃ alkylene.

As generally defined herein, m is 0, 1, or 2.

In some embodiments, m is 0.

In some embodiments, m is 1 or 2.

In some embodiments, m is 1. In some embodiments, m is 2.

### (c) Subgenera

It is understood that, for a compound of the present disclosure, variables Ring A, R¹, R^{1A}, R^{1B}, R^{1C}, R^{1D}, R^{1E}, R^{1F}, R², R^{2A}, R^{2B}, x, R³, R^{3A}, R^{3B}, R^{3C}, R^{3D}, R^{3E}, R⁴, R^{4A}, R^{4B}, R^{4C}, L₁, L₂ and L₃, and m can each be, where applicable, selected from the groups described herein, and any group described herein for any of variables Ring A, R¹, R^{1A}, R^{1B}, R^{1C}, R^{1D}, R^{1E}, R^{1F}, R², R^{2A}, R^{2B}, x, R³, R^{3A}, R^{3B}, R^{3C}, R^{3D}, R^{3E}, R⁴, R^{4A}, R^{4B}, R^{4C}, L₁, L₂ and L₃, and m can be combined, where applicable, with any group described herein for one or more of the remainder of variables Ring A, R¹, R^{1A}, R^{1B}, R^{1C}, R^{1D}, R^{1E}, R^{1F}, R², R^{2A}, R^{2B}, x, R³, R^{3A}, R^{3B}, R^{3C}, R^{3D}, R^{3E}, R⁴, R^{4A}, R^{4B}, R^{4C}, L₁, L₂ and L₃, and m. Additional exemplary combinations of the above described embodiments are further contemplated herein.

For example, in some embodiments, wherein Ring A is a group of formula (ii-b), the compound of Formula (I) is of Formula (I-a):

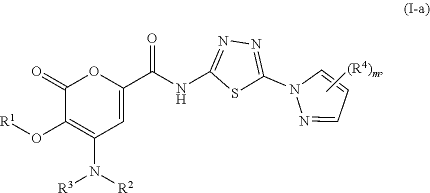

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-a) wherein R¹ is methyl, R² is hydrogen, and m is 1 or 2.

In some embodiments, wherein Ring A is a group of formula (iv-b), the compound of Formula (I) is of Formula (I-b):

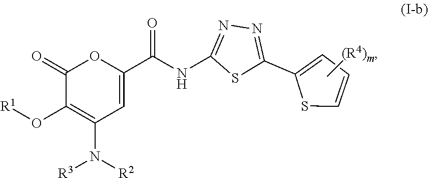

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-b) wherein R¹ is methyl, R² is hydrogen, and m is 1 or 2.

In some embodiments, wherein Ring A is a group of formula (v-b), the compound of Formula (I) is of Formula (I-c):

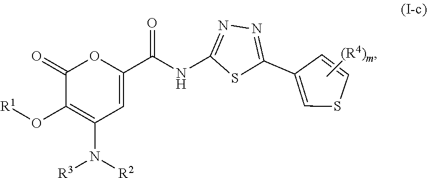

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-c) wherein R¹ is methyl, R² is hydrogen, and m is 1 or 2.

In some embodiments, wherein Ring A is a group of formula (i-b), the compound of Formula (I) is of Formula (I-d):

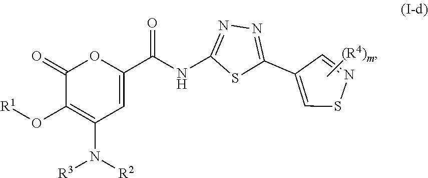

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-d) wherein R¹ is methyl, R² is hydrogen, and m is 1 or 2.

In some embodiments, wherein Ring A is a group of formula (vii-b), the compound of Formula (I) is of Formula (I-e):

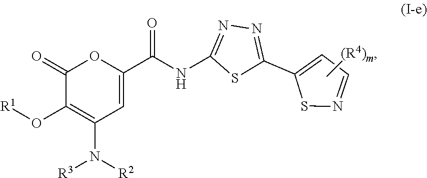

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-e) wherein R¹ is methyl, R² is hydrogen, and m is 1 or 2.

In some embodiments, wherein Ring A is a group of formula (xiii-b), the compound of Formula (I) is of Formula (I-f):

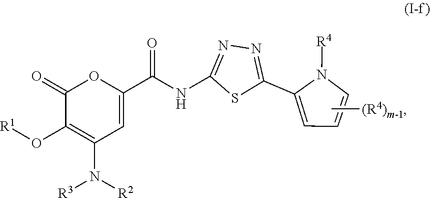

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-f) wherein R¹ is methyl, R² is hydrogen, and m is 1 or 2.

In some embodiments, wherein Ring A is a group of formula (xiv-b), the compound of Formula (I) is of Formula (I-g):

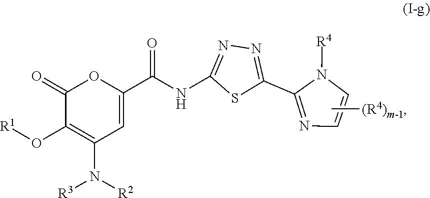

or a pharmaceutically acceptable salt thereof. In certain embodiments, the amino moiety at the C₄ position is a group of formula (i-a). In certain embodiments, the amino moiety at the C₄ position is a group of formula (i-b). In certain embodiments, the amino moiety at the C₄ position is a group of formula (i-c). In certain embodiments, R¹ is methyl (—CH₃). In certain embodiments, R² is hydrogen. In certain embodiments, m is 1 or 2. In some embodiments, the compound is of Formula (I-g) wherein R¹ is methyl, R² is hydrogen, and m is 1 or 2.

In some embodiments, wherein Ring A is a group of formula (ii-b) and the amino moiety at the C₄ position is a group of formula (i-a), the compound of Formula (I) is of Formula (I-a-1):

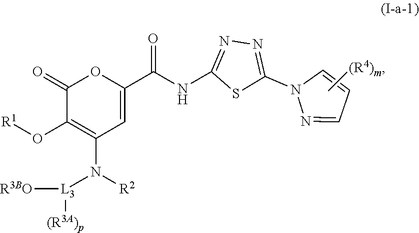

or a pharmaceutically acceptable salt thereof, wherein L₃ is C₁-C₁₀ alkylene, C₂-C₁₀ alkenylene, or C₁-C₁₀ alkynylene, and p is 0, 1, 2, or 3. In some embodiments, the compound is of Formula (I-a-1) wherein R¹ is methyl, R² is hydrogen, and m is 1 or 2.

In some embodiments, wherein Ring A is a group of formula (ii-b) and the amino moiety at the C₄ position is a group of formula (ii-a), the compound of Formula (I is of Formula (I-a-2):

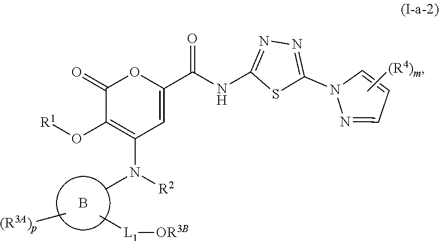

or a pharmaceutically acceptable salt thereof, wherein Ring B is C₃-C₁₀ carbocyclyl, or 4- to 10-membered heterocyclyl, and p is 0, 1, 2, or 3. In some embodiments, the compound is of Formula (I-a-2) wherein R¹ is methyl, R² is hydrogen, and m is 1 or 2.

In some embodiments, wherein Ring A is a group of formula (ii-b) and the amino moiety at the C₄ position is a group of formula (iii-a), the compound of Formula (I) is of Formula (I-a-3):

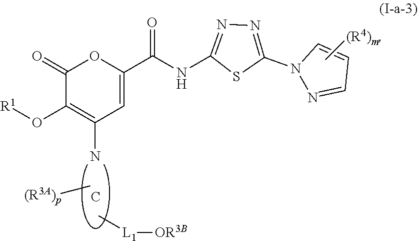

or a pharmaceutically acceptable salt thereof, wherein Ring C is a 4- to 10-membered heterocyclyl and p is 0, 1, 2, or 3. In some embodiments, the compound is of Formula (I-a-3) wherein R¹ is methyl and m is 1 or 2.

In some embodiments, wherein Ring A is a group of formula (xiii-b) and the amino moiety at the C₄ position is a group of formula (i-a), the compound of Formula (I) is of Formula (I-f-1):

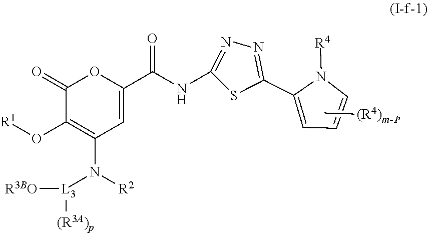

or a pharmaceutically acceptable salt thereof, wherein L₃ is C₁-C₁₀ alkylene, C₂-C₁₀ alkenylene, or C₂-C₁₀ alkynylene, and p is 0, 1, 2, or 3. In some embodiments, the compound is of Formula (I-f-1) wherein R¹ is methyl, R² is hydrogen, and m is 1 or 2.

In some embodiments, wherein Ring A is a group of formula (xiii-b) and the amino moiety at the C₄ position is a group of formula (ii-a), the compound of Formula (I) is of Formula (I-f-2):

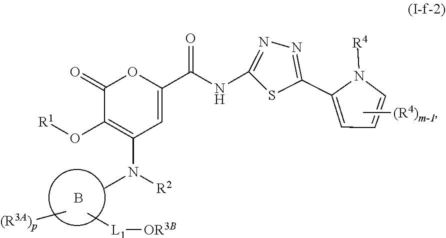

or a pharmaceutically acceptable salt thereof, wherein Ring B is C₃-C₁₀ carbocyclyl, or 4- to 10-membered heterocyclyl, and p is 0, 1, 2, or 3. In some embodiments, the compound is of Formula (I-f-2) wherein R¹ is methyl, R² is hydrogen, and m is 1 or 2.

In some embodiments, wherein Ring A is a group of formula (xiii-b) and the amino moiety at the C₄ position is a group of formula (iii-a), the compound of Formula (I) is of Formula (I-f-3):

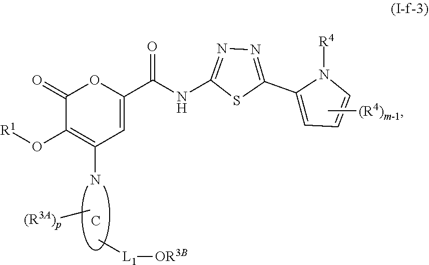

or a pharmaceutically acceptable salt thereof, wherein Ring C is a 4- to 10-membered heterocyclyl and p is 0, 1, 2, or 3. In some embodiments, the compound is of Formula (I-f-3) wherein R¹ is methyl and m is 1 or 2.

In some embodiments, wherein R¹ and R² are cyclized to form a 6-membered heterocyclic ring, provided is a compound of Formula (I-BC-a) or (I-BC-b):

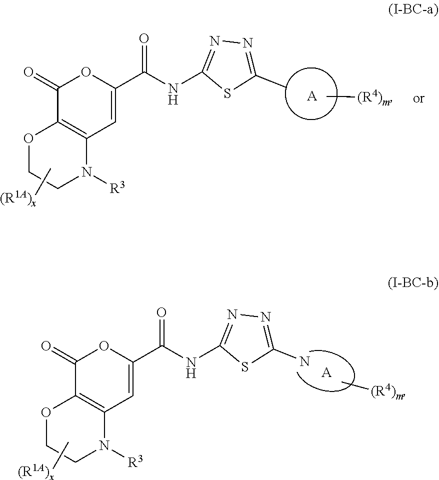

or a pharmaceutically acceptable salt thereof, wherein x is 0, 1, 2, 3, or 4.

In some embodiments, the compound of Formula (I) is selected from any one of the compounds of Table 1, or a pharmaceutically acceptable salt thereof.

In some embodiments, the compound of Formula (I) is selected from any one of the compounds of Table 2, or a pharmaceutically acceptable salt thereof.

In some embodiments, the compound of Formula (I) is selected from a pharmaceutically acceptable salt of any one of the compounds of Table 1 or Table 2.

In some embodiments, the compound of Formula (I) is a free base selected from any one of the compounds of Table 1 or Table 2.

The below Table 1 and Table 2 also provides the location of the compound in the Examples (Ex) by Example Number (Ex) or as provided in Table A (TA) of the Examples. The Asterix (*) next to the Compound Number (#) signifies that arbitrary stereochemistry has been assigned.

TABLE 1
Compounds of Formula (I)

| Ex | # | Compound structure and name |
|---|---|---|
| TA | 1 | 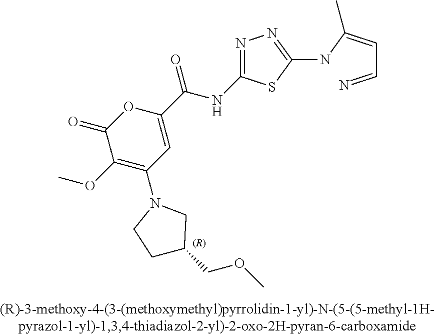 |
| TA | 2 | 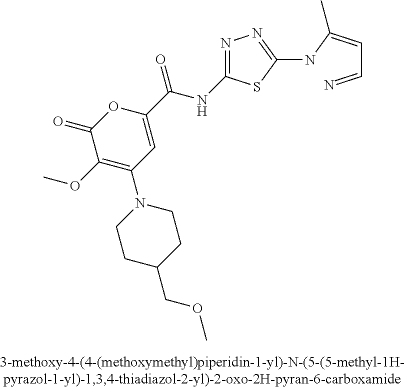 [SMILES: COCC1CCN(c2cc(C(=O)Nc3nnc(N/C(C)=C\C=N)s3)oc(=O)c2OC)CC1] |
| 2 | 3* | 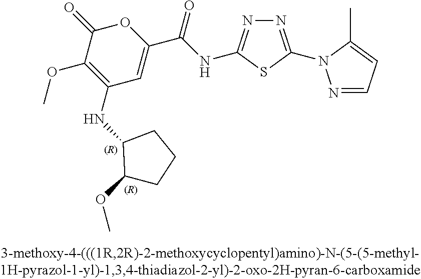 |
| 2 | 4* | 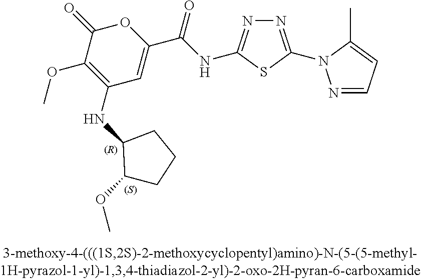 |
| TA | 5 | 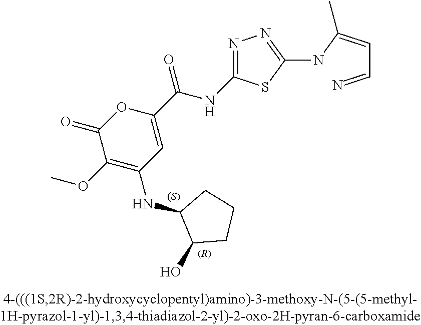 |
| TA | 6a* | 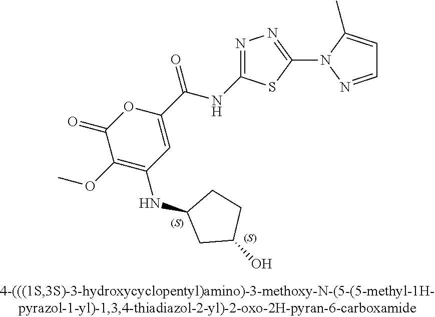 |
| TA | 6b* | 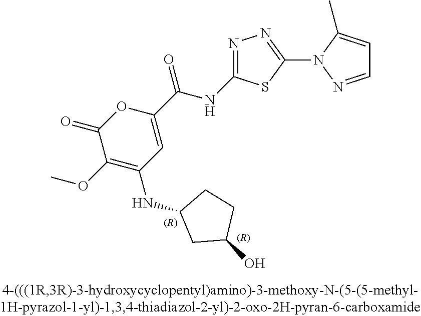 |
| 5 | 7a* | 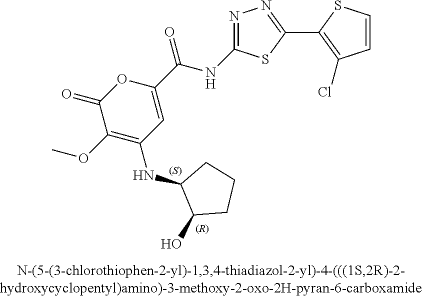 |
| 5 | 7b* | 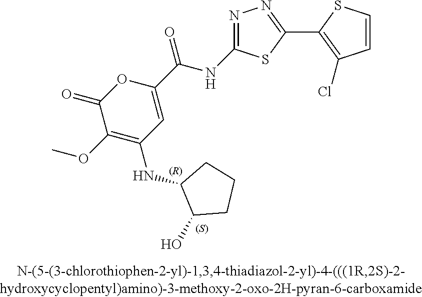 |
| TA | 8 | 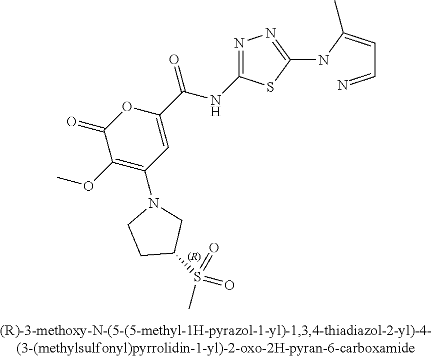 |
| TA | 9 | 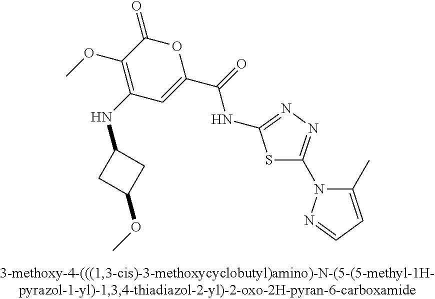 |
| 1 | 10 | 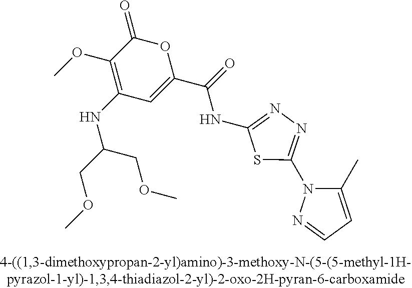 |
| TA | 11 | 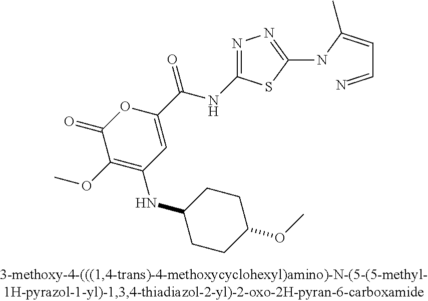 |
| TA | 12 | 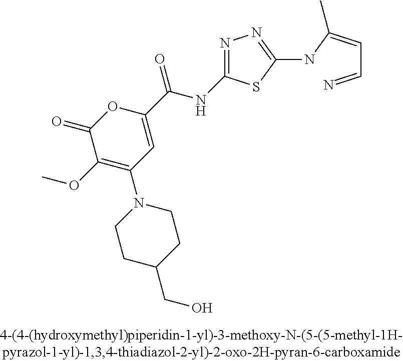 [SMILES: COc1c(N2CCC(CO)CC2)cc(C(=O)Nc2nnc(N/C(C)=C\C=N)s2)oc1=O] |
| TA | 13 | 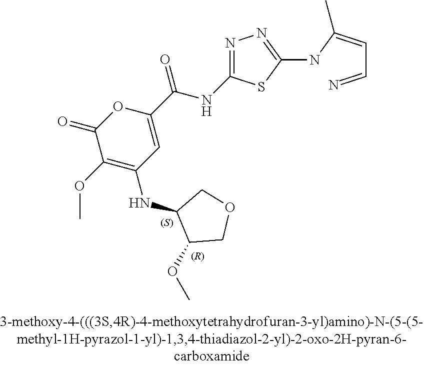 |
| TA | 14a* | 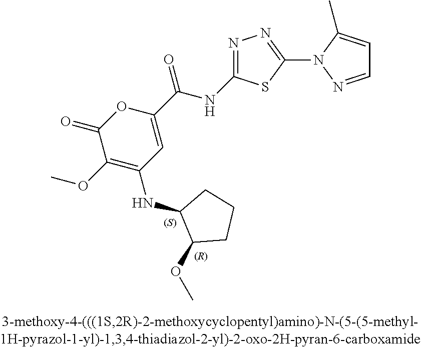 |
| TA | 14b* | 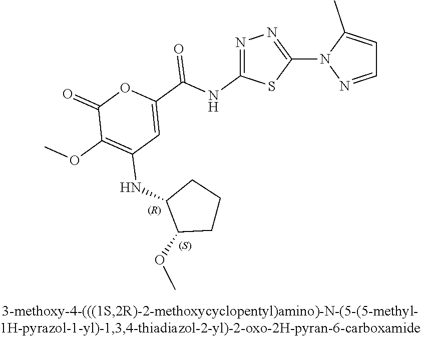 |
| TA | 15a* | 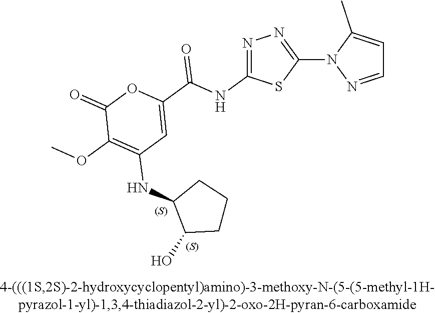 |
| TA | 15b* | 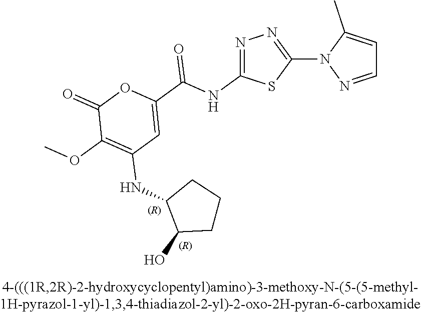 |
| TA | 16 | 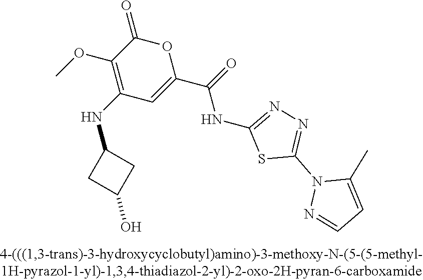 |
| TA | 17 | 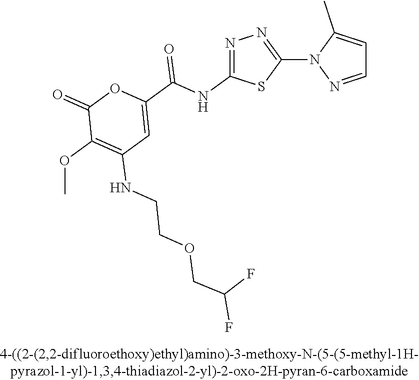 [SMILES: COc1c(NCCOCC(F)F)cc(C(=O)Nc2nnc(-n3nccc3C)s2)oc1=O] |
| TA | 18 | 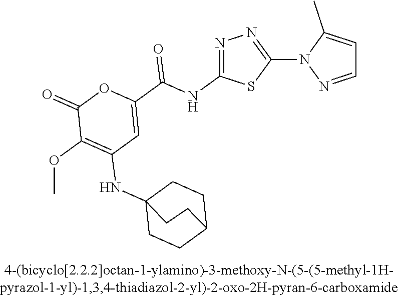 [SMILES: COc1c(NC23CCC(CC2)CC3)cc(C(=O)Nc2nnc(-n3nccc3C)s2)oc1=O] |
| TA | 19 | 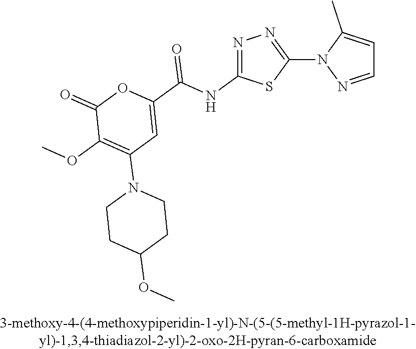 [SMILES: COc1c(N2CCC(OC)CC2)cc(C(=O)Nc2nnc(-n3nccc3C)s2)oc1=O] |
| TA | 20 | 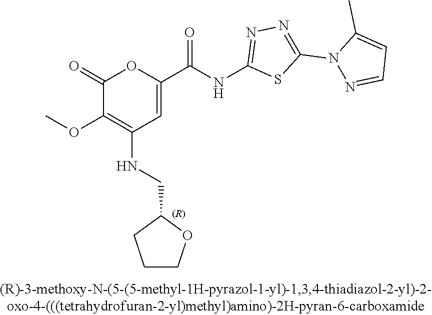 |
| 6 | 21* | 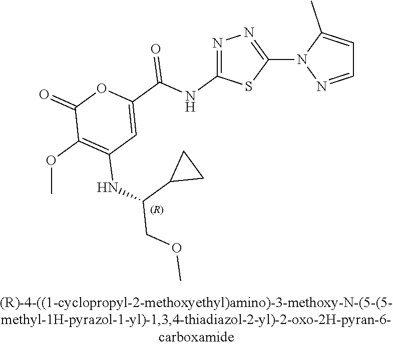 |
| 6 | 22* | 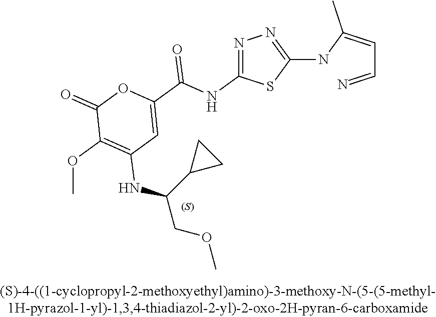 |
| 3 | 23a* | 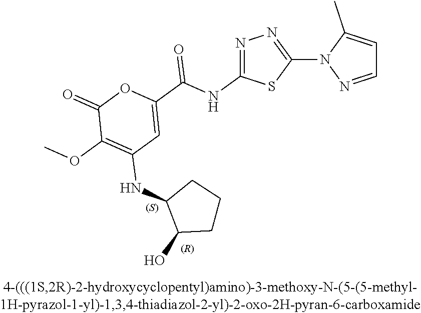 |
| 3 | 23b* | 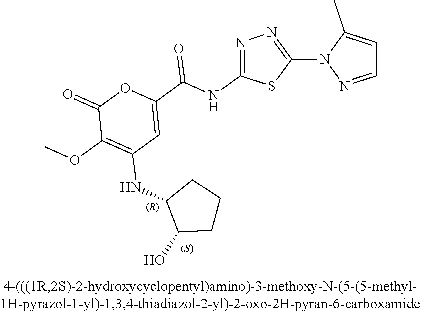 |
| TA | 24 | 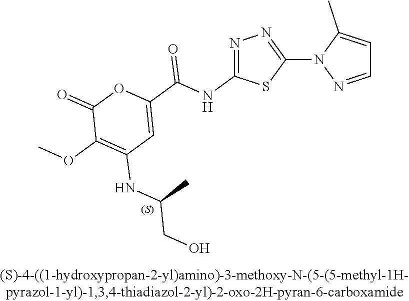 |
| TA | 25 | 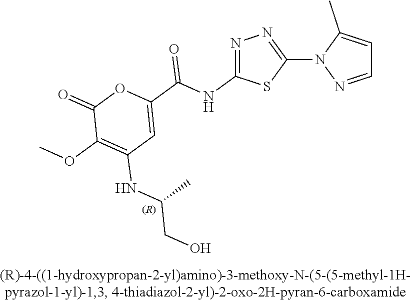 |
| 7 | 26a* | 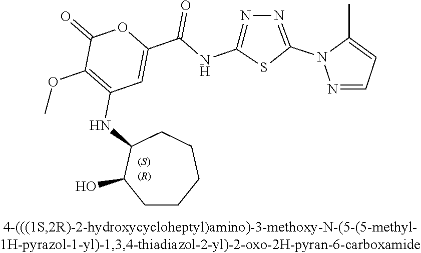 |
| 7 | 26b* | 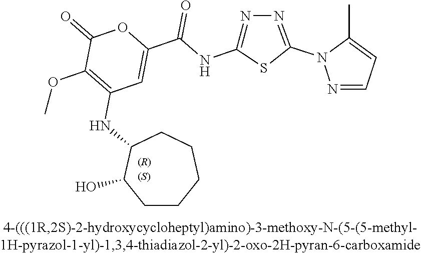 |
| TA | 27 | 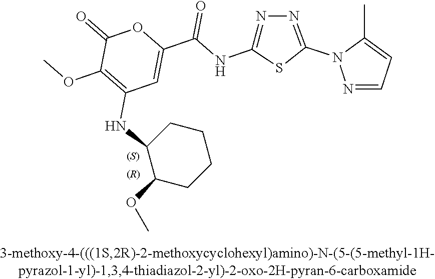 |
| 8 | 28a* | 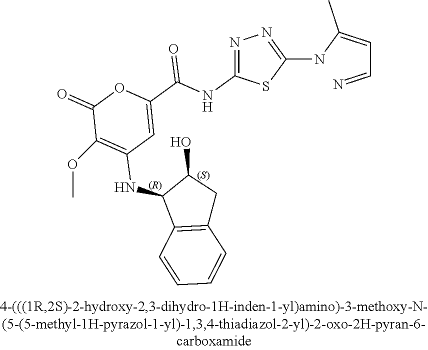 |
| 8 | 28b* | 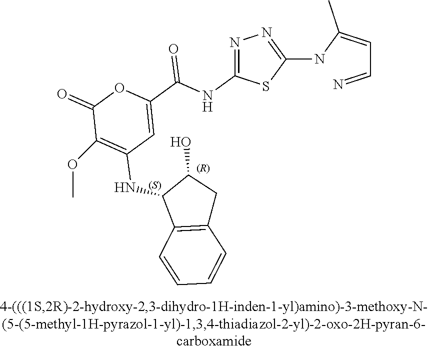 |
| 9 | 29 | 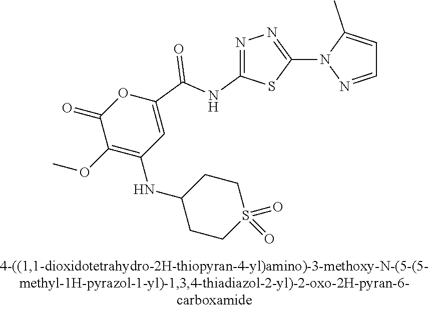 [SMILES: COc1c(NC2CCS(=O)(=O)CC2)cc(C(=O)Nc2nnc(-n3nccc3C)s2)oc1=O] |
| TA | 30 | 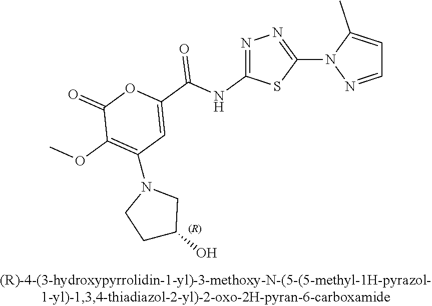 |
| TA | 31 | 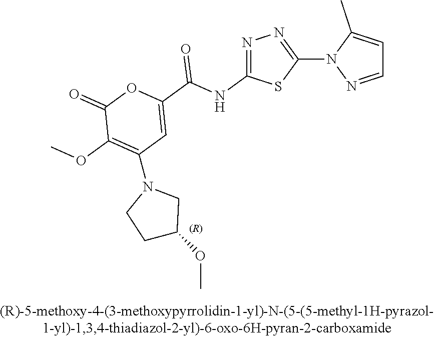 |
| TA | 32 | 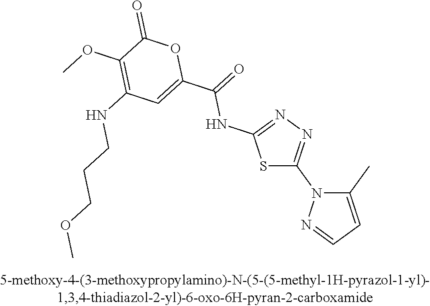 |
| TA | 33 | 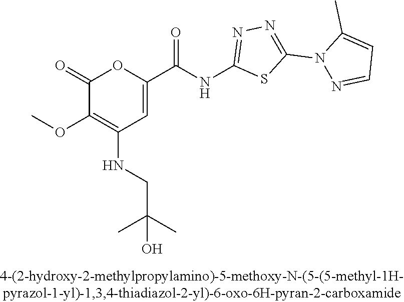 [SMILES: COc1c(NCC(C)(C)O)cc(C(=O)Nc2nnc(-n3nccc3C)s2)oc1=O] |
| TA | 34 | 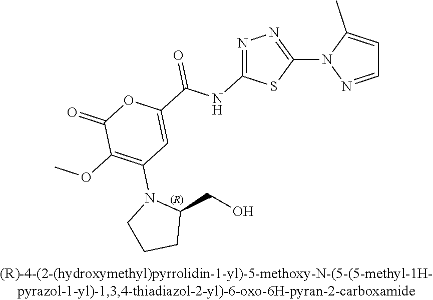 |
| TA | 35 | 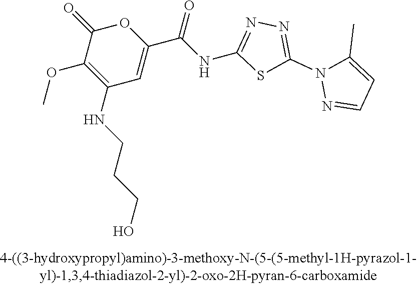 [SMILES: COc1c(NCCCO)cc(C(=O)Nc2nnc(-n3nccc3C)s2)oc1=O] |
| TA | 36a* | 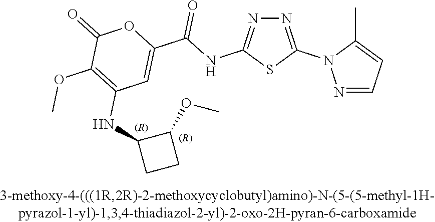 |
| TA | 36b* | 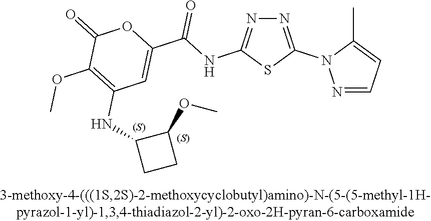 |
| TA | 37a* | 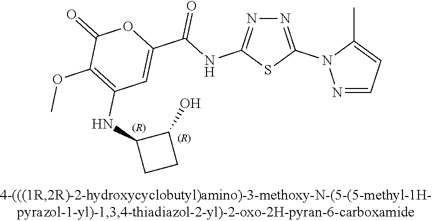 |
| TA | 37b* | 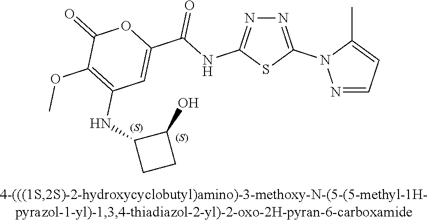 |
| TA | 38 | 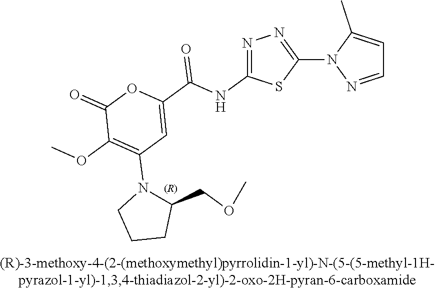 |
| TA | 39 | 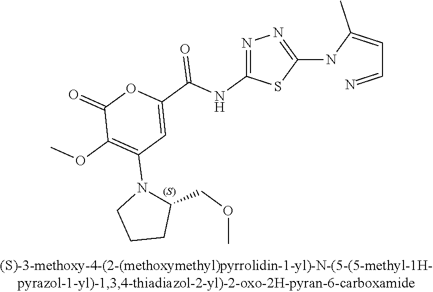 |
| TA | 40 | 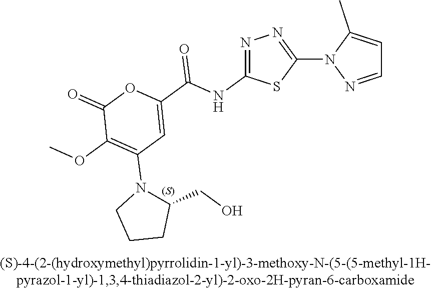 |
| TA | 41 | 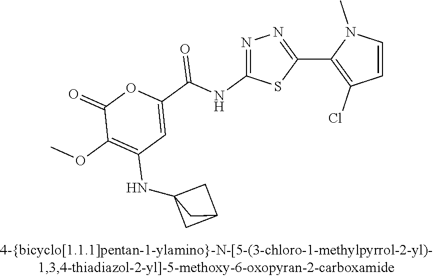 [SMILES: COc1c(NC23CC(C2)C3)cc(C(=O)Nc2nnc(-c3c(Cl)ccn3C)s2)oc1=O] |
| TA | 42 | 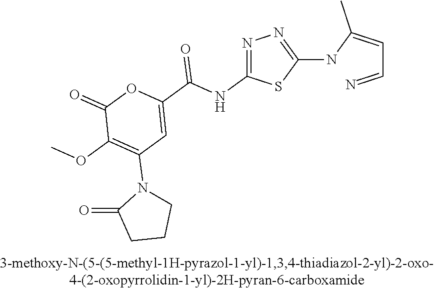 [SMILES: COc1c(N2CCCC2=O)cc(C(=O)Nc2nnc(N/C(C)=C\C=N)s2)oc1=O] |
| TA | 43a* | 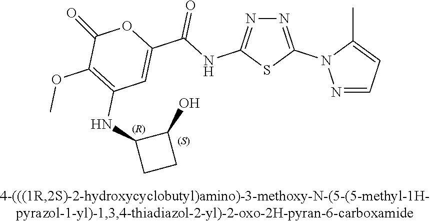 |
| TA | 43b* | 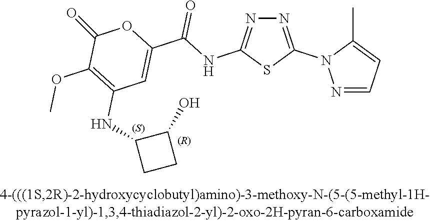 |
| TA | 44 | 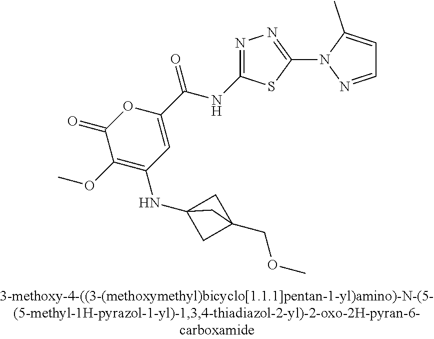 [SMILES: COCC12CC(Nc3cc(C(=O)Nc4nnc(-n5nccc5C)s4)oc(=O)c3OC)(C1)C2] |
| TA | 45 | 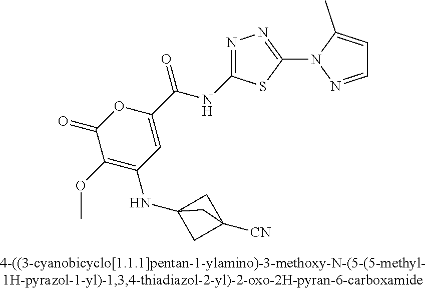 [SMILES: COc1c(NC23CC(C#N)(C2)C3)cc(C(=O)Nc2nnc(-n3nccc3C)s2)oc1=O] |
| TA | 46 | 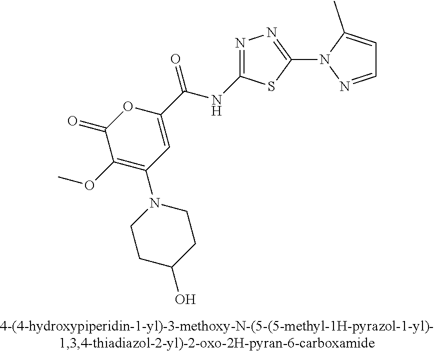 [SMILES: COc1c(N2CCC(O)CC2)cc(C(=O)Nc2nnc(-n3nccc3C)s2)oc1=O] |
| TA | 47* | 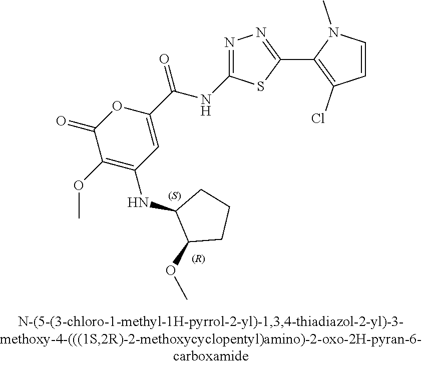 |
| TA | 48* | 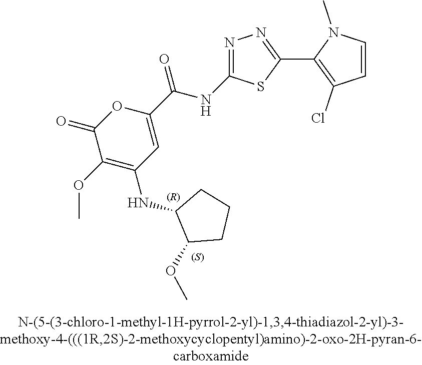 |
| TA | 49 | 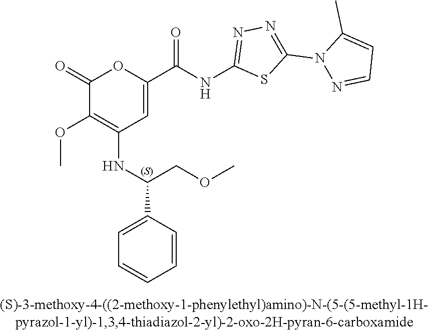 |
| TA | 50 | 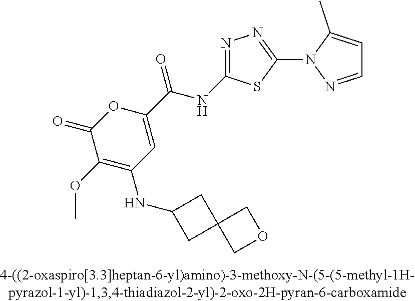 [SMILES: COc1c(NC2CC3(COC3)C2)cc(C(=O)Nc2nnc(-n3nccc3C)s2)oc1=O] |
| TA | 51 | 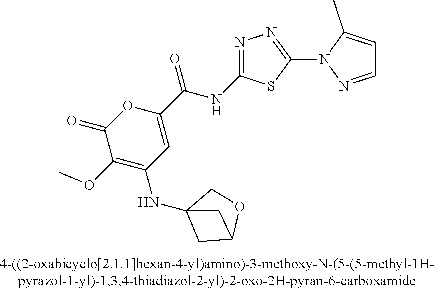 [SMILES: COc1c(NC23COC(C2)C3)cc(C(=O)Nc2nnc(-n3nccc3C)s2)oc1=O] |
| TA | 52 | 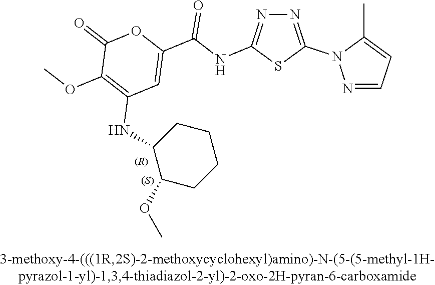 |
| 11 | 53 | 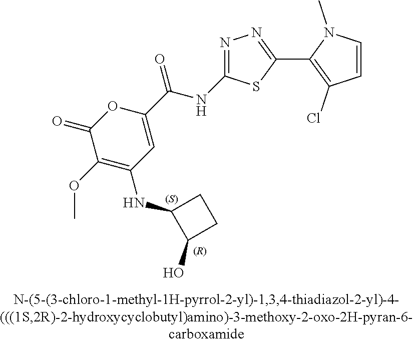 |
| 11 | 54 | 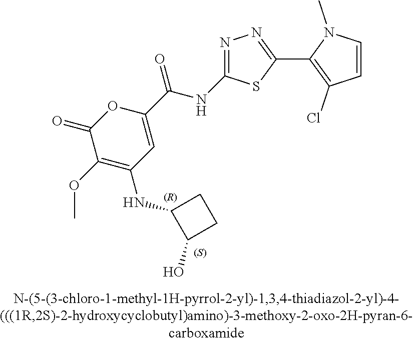 |
| TA | 55a* | 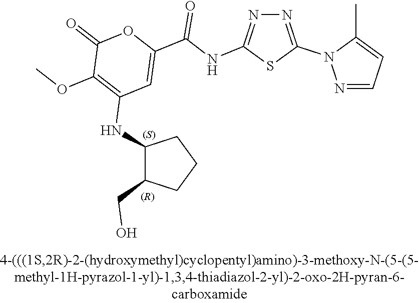 |
| TA | 55b* | 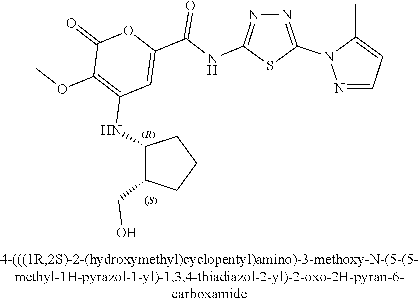 |
| 63 | 56 | 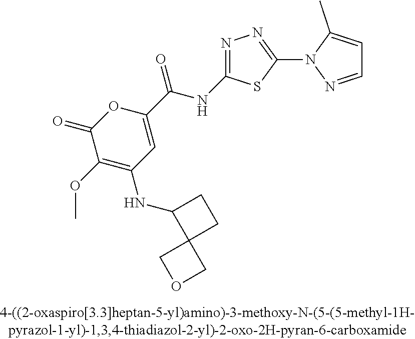 [SMILES: COc1c(NC2CCC23COC3)cc(C(=O)Nc2nnc(-n3nccc3C)s2)oc1=O] |
| 63 | 56a* | 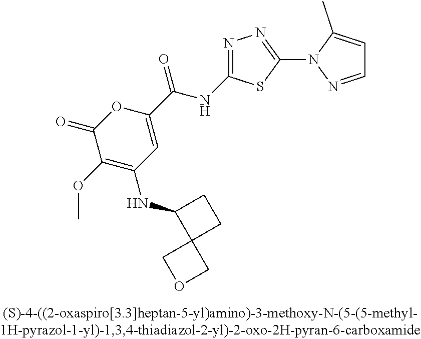 [SMILES: COc1c(N[C@H]2CCC23COC3)cc(C(=O)Nc2nnc(-n3nccc3C)s2)oc1=O] |
| TA | 57 | 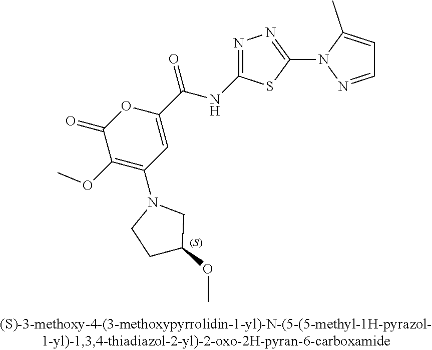 |
| TA | 58 | 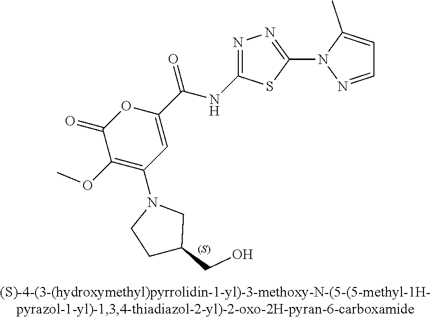 |
| TA | 59 | 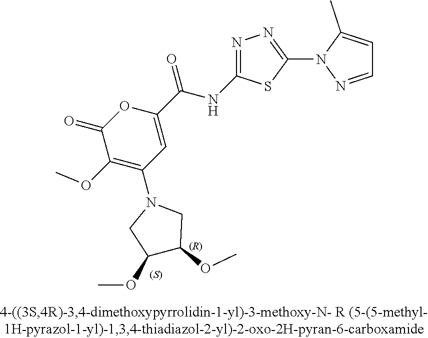 |
| TA | 60 | 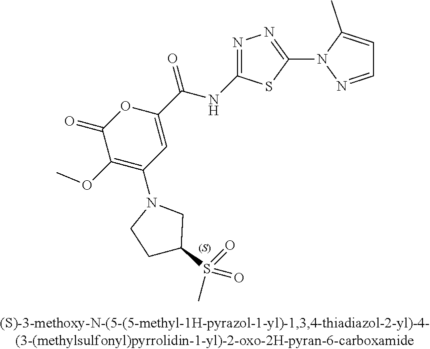 |
| TA | 61a* | 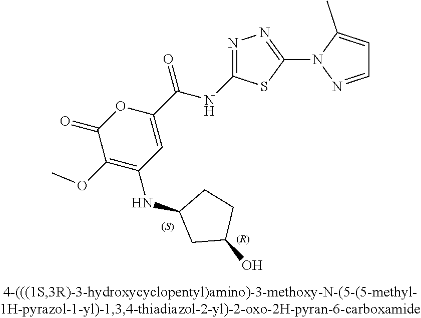 |
| TA | 61b* | 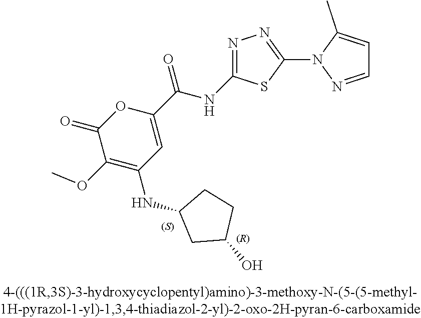 |
| TA | 62 | 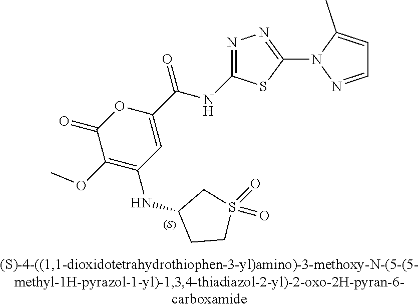 |
| TA | 63 | 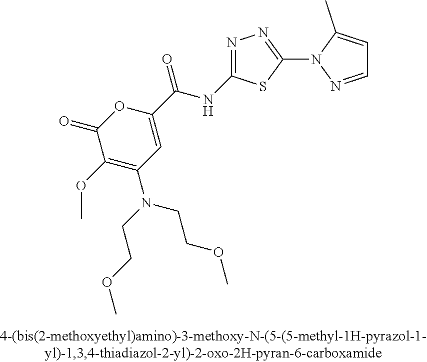 [SMILES: COCCN(CCOC)c1cc(C(=O)Nc2nnc(-n3nccc3C)s2)oc(=O)c1OC] |
| TA | 64 | 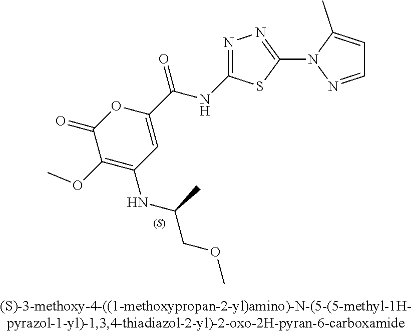 |
| TA | 65 | 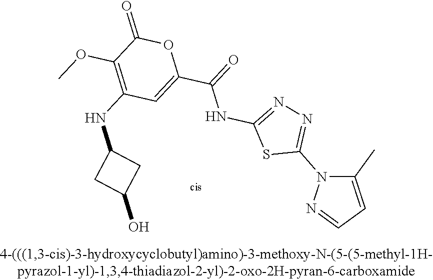 |
| TA | 66 | 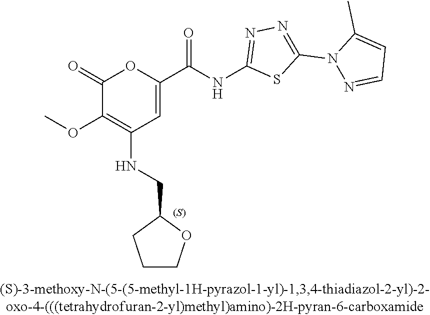 |
| TA | 67a* | 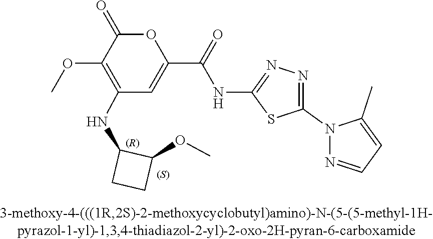 |
| TA | 67b* | 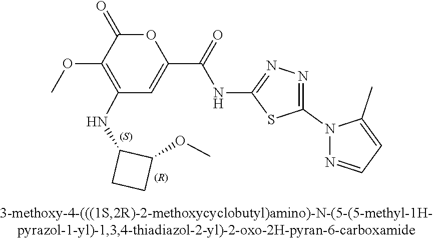 |
| TA | 68a* | 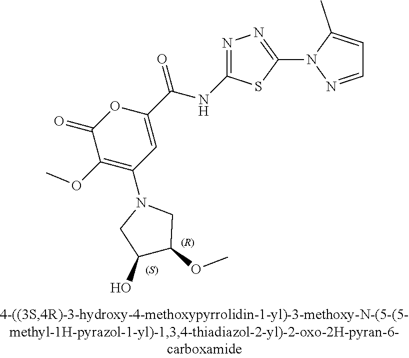 |
| TA | 68b* | 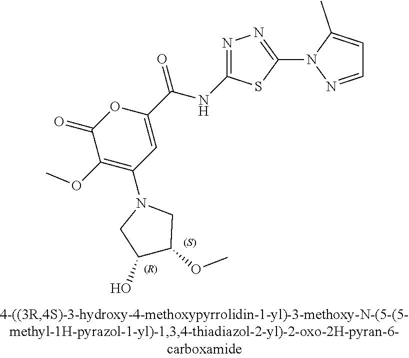 |
| TA | 69 | 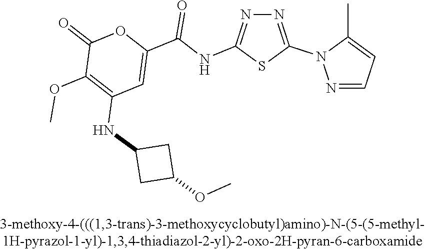 |
| TA | 70 | 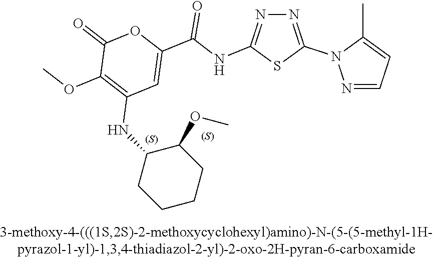 |
| TA | 71* | 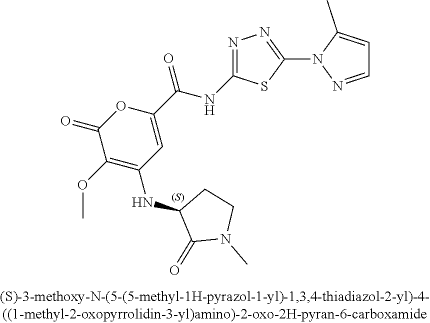 |
| TA | 72* | 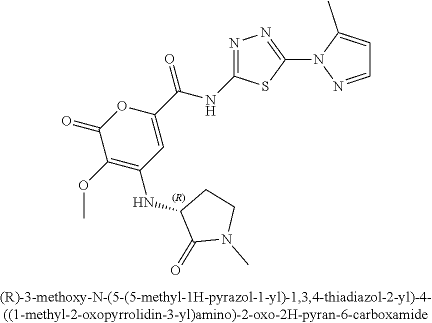 |
| 12 | 73 | 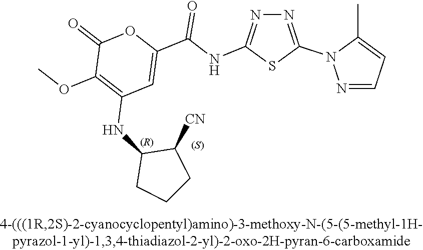 |
| 12 | 74 | 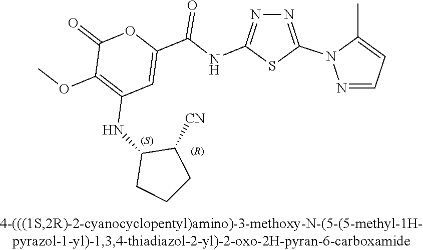 |
| 13 | 75 | 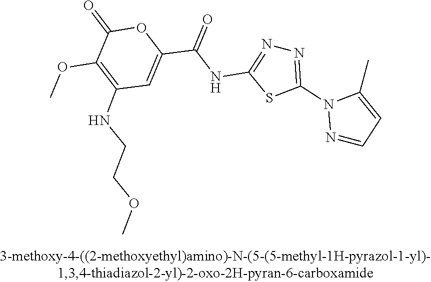 [SMILES: COCCNc1cc(C(=O)Nc2nnc(-n3nccc3C)s2)oc(=O)c1OC] |
| TA | 76 | 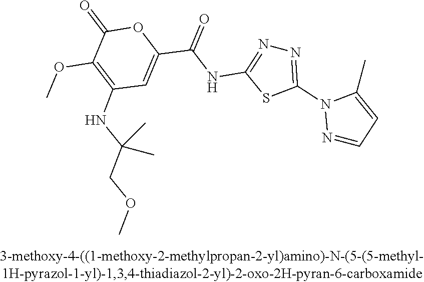 [SMILES: COCC(C)(C)Nc1cc(C(=O)Nc2nnc(-n3nccc3C)s2)oc(=O)c1OC] |
| TA | 77 | 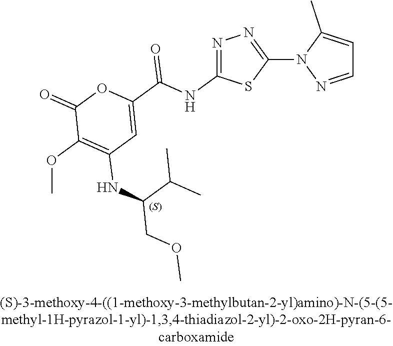 |
| TA | 78 | 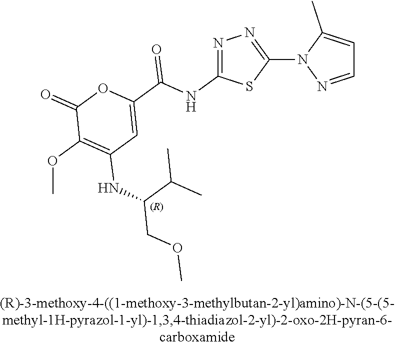 |
| 14 | 79* | 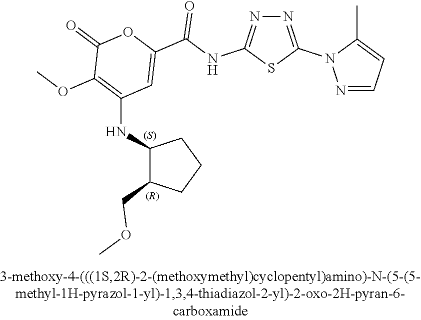 |
| 14 | 80* | 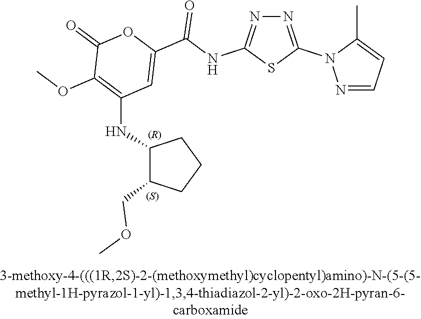 |
| TA | 81a* | 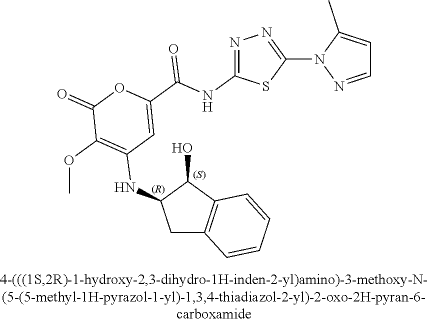 |
| TA | 81b* | 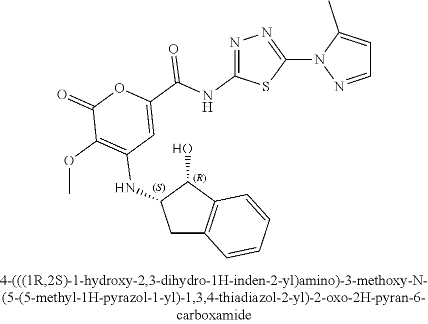 |
| TA | 82 | 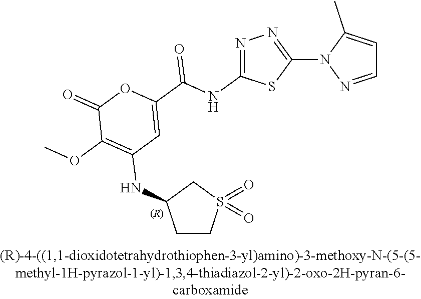 |
| TA | 83 | 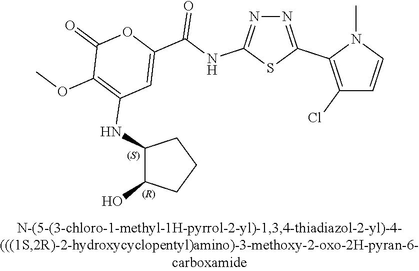 |
| TA | 84 | 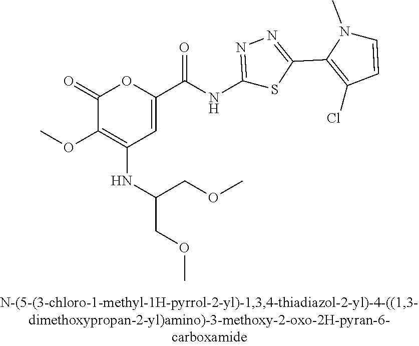 [SMILES: COCC(COC)Nc1cc(C(=O)Nc2nnc(-c3c(Cl)ccn3C)s2)oc(=O)c1OC] |
| TA | 85 | 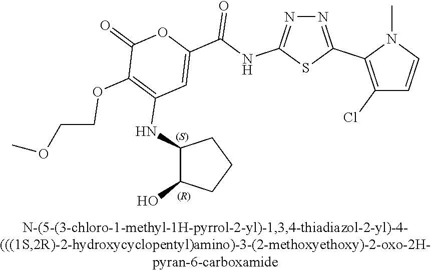 |
| TA | 86 | 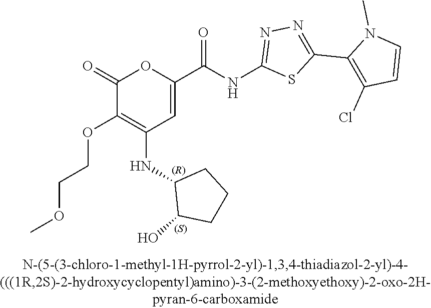 |
| 17 | 87 | 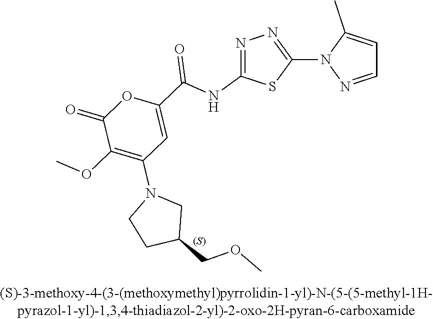 |
| TA | 88 | 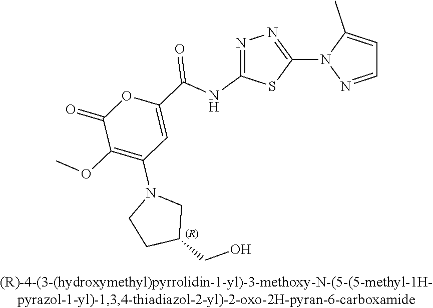 |
| TA | 89 | 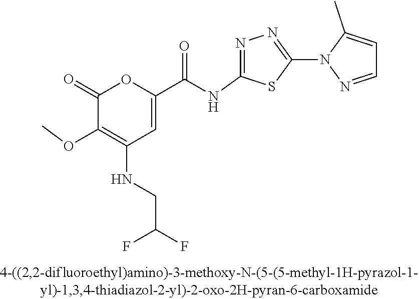 [SMILES: COc1c(NCC(F)F)cc(C(=O)Nc2nnc(-n3nccc3C)s2)oc1=O] |
| TA | 90 | 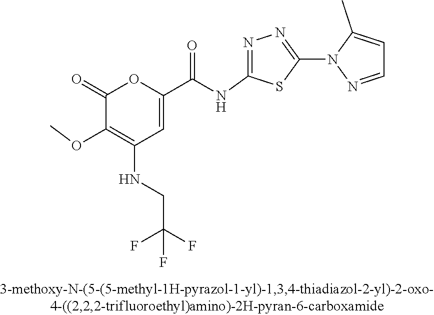 [SMILES: COc1c(NCC(F)(F)F)cc(C(=O)Nc2nnc(-n3nccc3C)s2)oc1=O] |
| TA | 91a* | 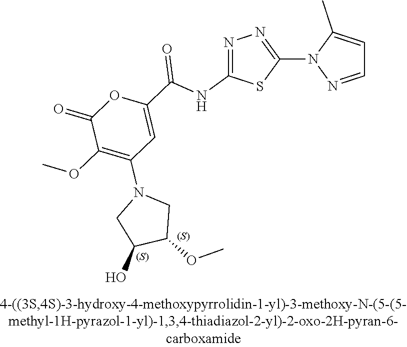 |
| TA | 91b* | 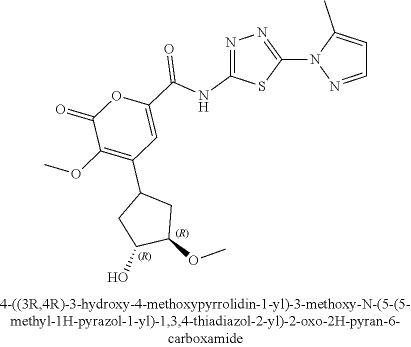 |
| TA | 92a* | 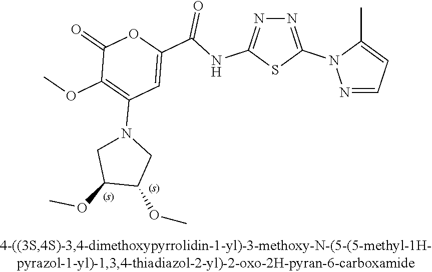 |
| TA | 92b* | 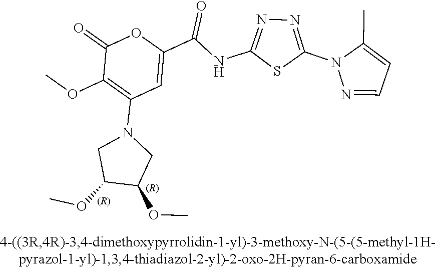 |
| TA | 93 | 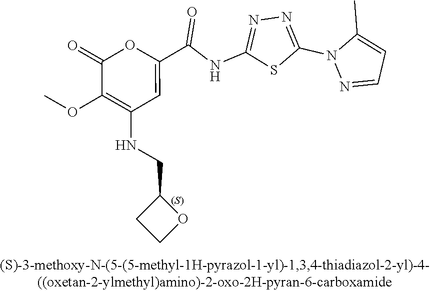 |
| 18 | 94 | 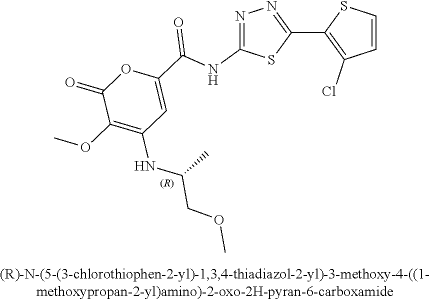 |
| TA | 95 | 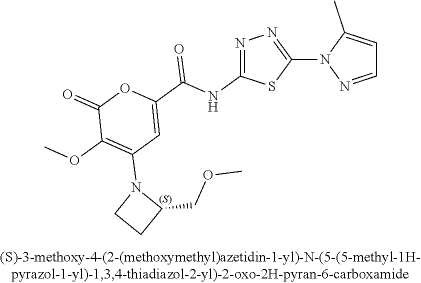 |
| TA | 96 | 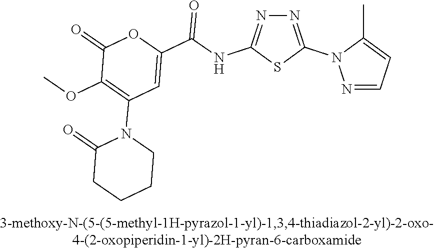 [SMILES: COc1c(N2CCCCC2=O)cc(C(=O)Nc2nnc(-n3nccc3C)s2)oc1=O] |
| TA | 97 | 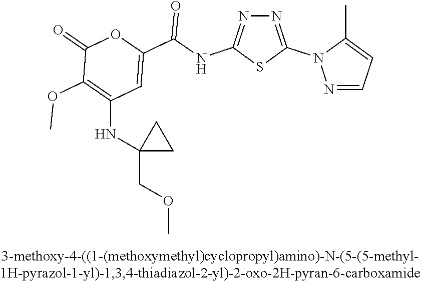 [SMILES: COCC1(Nc2cc(C(=O)Nc3nnc(-n4nccc4C)s3)oc(=O)c2OC)CC1] |
| TA | 98 | 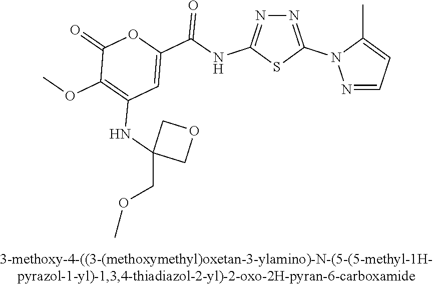 [SMILES: COCC1(Nc2cc(C(=O)Nc3nnc(-n4nccc4C)s3)oc(=O)c2OC)COC1] |
| TA | 99 | 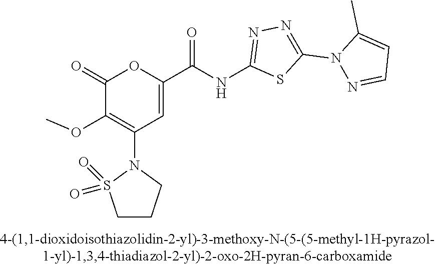 [SMILES: COc1c(N2CCCS2(=O)=O)cc(C(=O)Nc2nnc(-n3nccc3C)s2)oc1=O] |
| 19 | 100* | 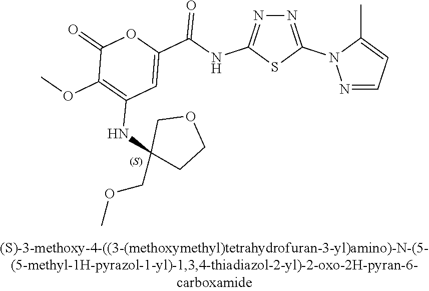 |
| 19 | 101* | 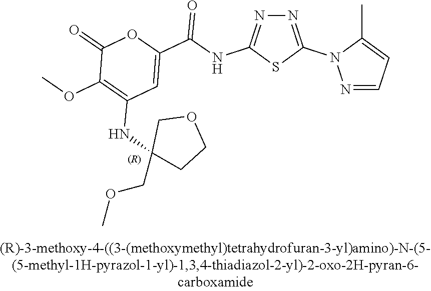 |
| TA | 102 | 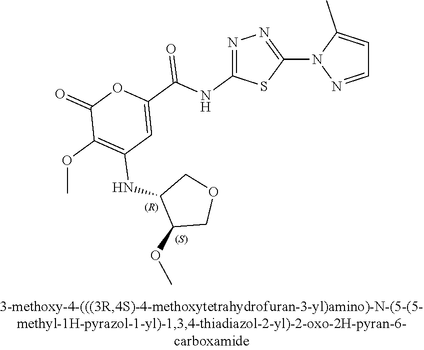 |
| TA | 103 | 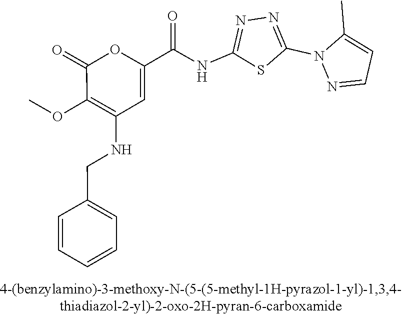 [SMILES: COc1c(NCc2ccccc2)cc(C(=O)Nc2nnc(-n3nccc3C)s2)oc1=O] |
| TA | 104 | 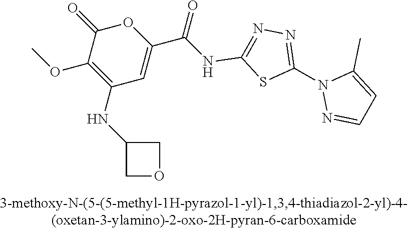 [SMILES: COc1c(NC2COC2)cc(C(=O)Nc2nnc(-n3nccc3C)s2)oc1=O] |
| TA | 105 | 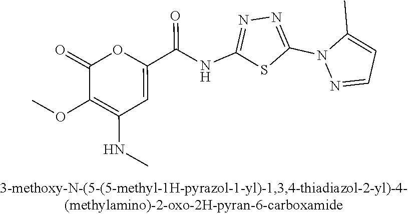 [SMILES: CNc1cc(C(=O)Nc2nnc(-n3nccc3C)s2)oc(=O)c1OC] |
| TA | 106 | 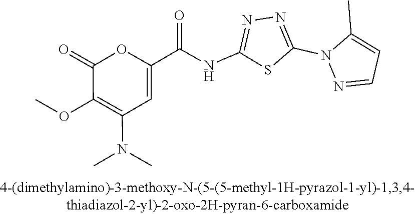 [SMILES: COc1c(N(C)C)cc(C(=O)Nc2nnc(-n3nccc3C)s2)oc1=O] |
| 50 | 174-OH | 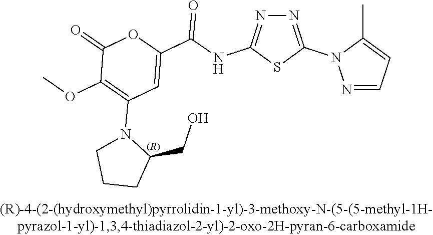 |
| 21 | 126-OMe | 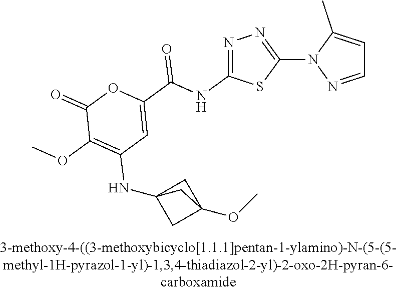 [SMILES: COc1c(NC23CC(OC)(C2)C3)cc(C(=O)Nc2nnc(-n3nccc3C)s2)oc1=O] |
| 22 | 129-OMe | 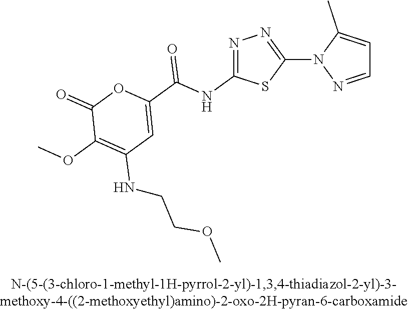 [SMILES: COCCNc1cc(C(=O)Nc2nnc(-n3nccc3C)s2)oc(=O)c1OC] |
| 59 | 210-Ac | 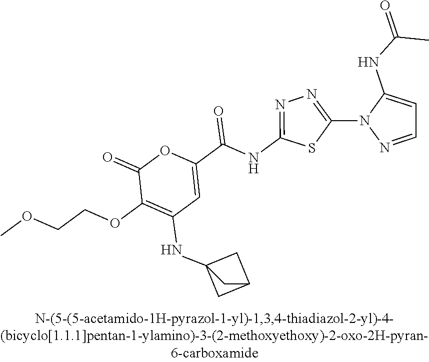 [SMILES: COCCOc1c(NC23CC(C2)C3)cc(C(=O)Nc2nnc(-n3nccc3NC(C)=O)s2)oc1=O] |
| TA | 107a* | 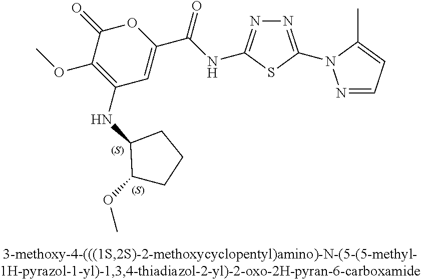 |
| TA | 107b* | 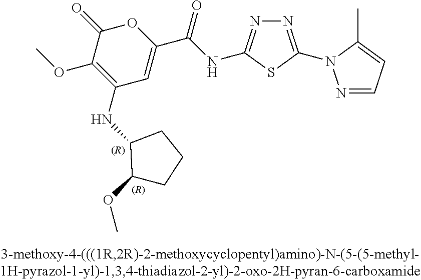 |
| 20 | 108a* | 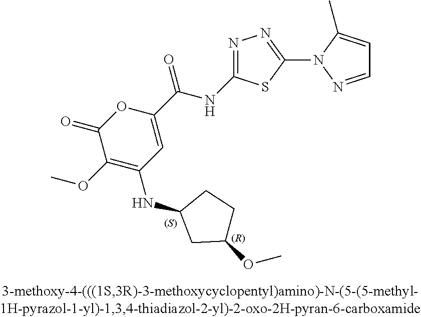 |
| 20 | 108b* | 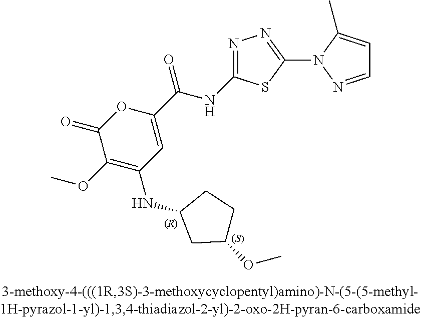 |
| 20 | 109a* | 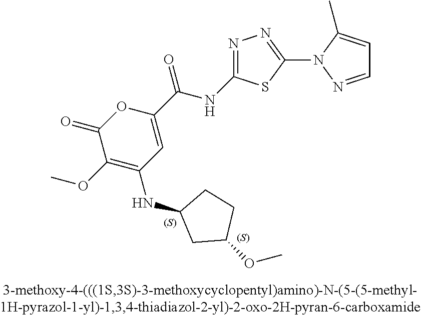 |
| 20 | 109b* | 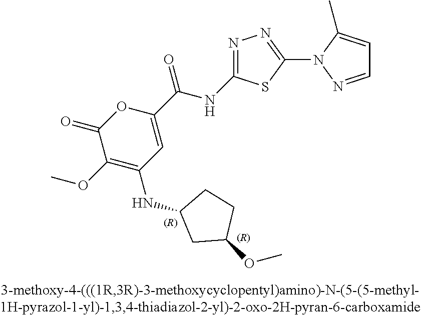 |
| TA | 110 | 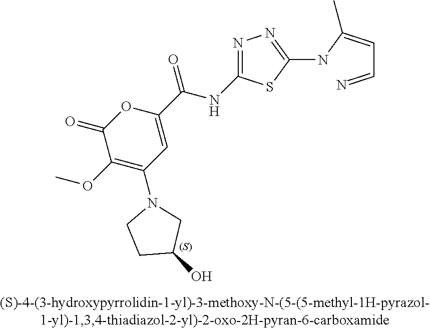 |
| TA | 111 | 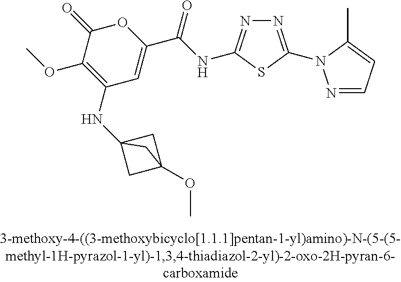 [SMILES: COc1c(NC23CC(OC)(C2)C3)cc(C(=O)Nc2nnc(-n3nccc3C)s2)oc1=O] |
| TA | 112 | 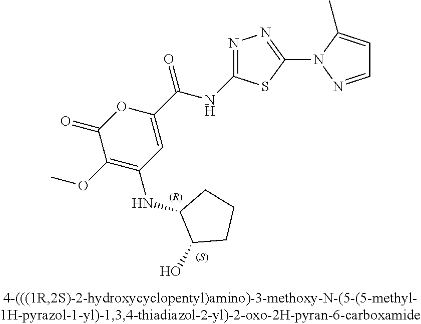 |
| TA | 113 | 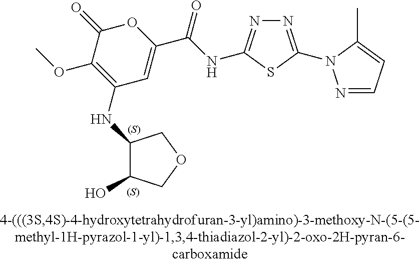 |
| TA | 114 | 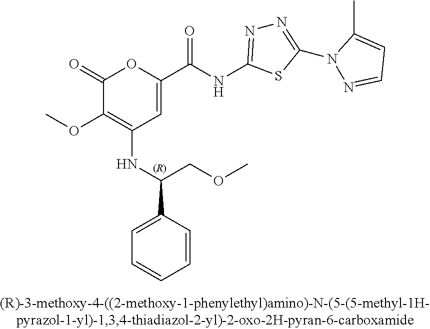 |
| TA | 115 | 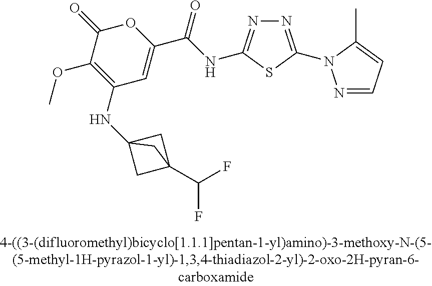 [SMILES: COc1c(NC23CC(C(F)F)(C2)C3)cc(C(=O)Nc2nnc(-n3nccc3C)s2)oc1=O] |
| TA | 116 | 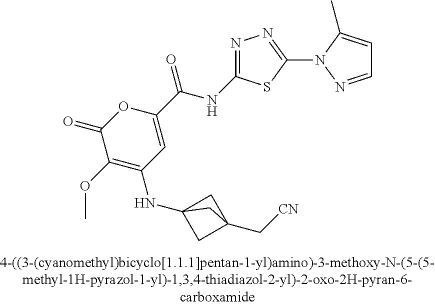 [SMILES: COc1c(NC23CC(CC#N)(C2)C3)cc(C(=O)Nc2nnc(-n3nccc3C)s2)oc1=O] |
| TA | 117 | 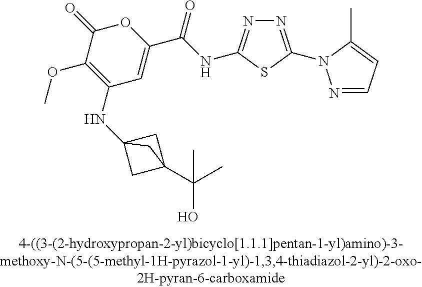 [SMILES: COc1c(NC23CC(C(C)(C)O)(C2)C3)cc(C(=O)Nc2nnc(-n3nccc3C)s2)oc1=O] |
| TA | 118 | 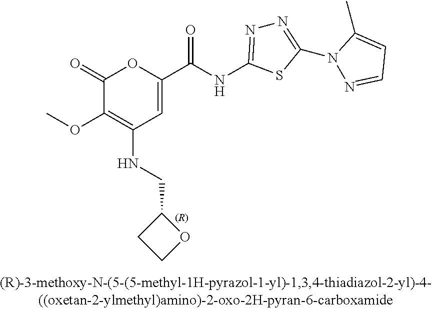 |
| TA | 119 | 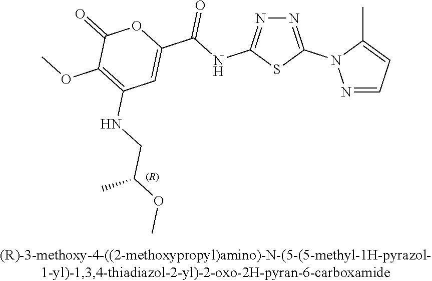 |
| TA | 120 | 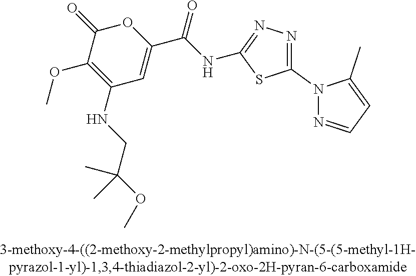 [SMILES: COc1c(NCC(C)(C)OC)cc(C(=O)Nc2nnc(-n3nccc3C)s2)oc1=O] |
| TA | 121 | 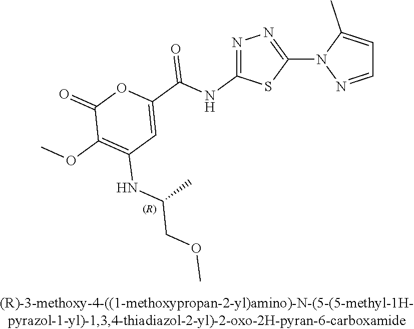 |
| TA | 122* | 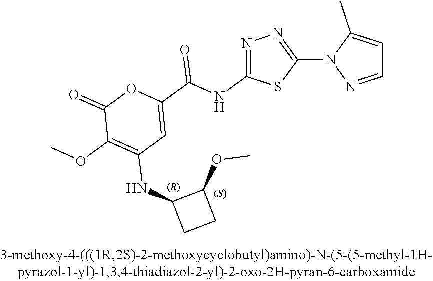 |
| TA | 123* | 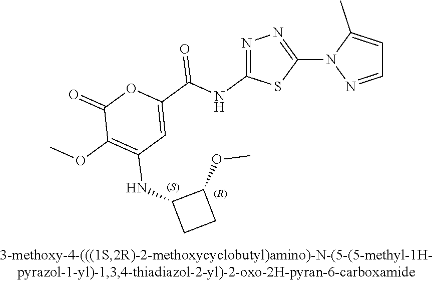 |
| TA | 124* | 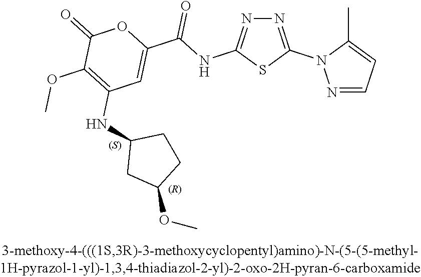 |
| TA | 125* | 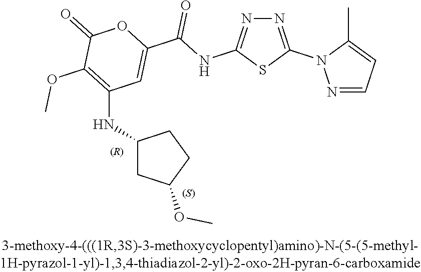 |
| 21 | 126 | 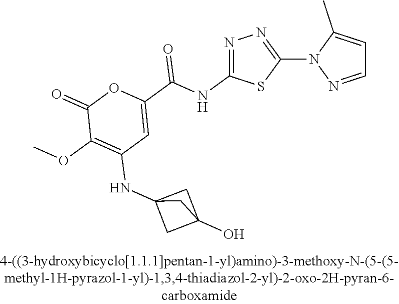 [SMILES: COc1c(NC23CC(O)(C2)C3)cc(C(=O)Nc2nnc(-n3nccc3C)s2)oc1=O] |
| TA | 127 | 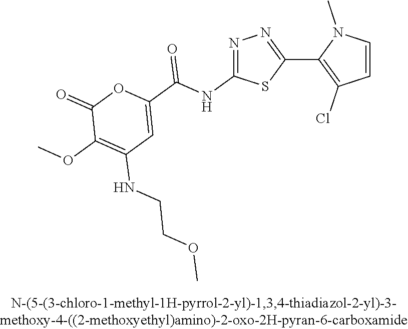 [SMILES: COCCNc1cc(C(=O)Nc2nnc(-c3c(Cl)ccn3C)s2)oc(=O)c1OC] |
| TA | 128 | 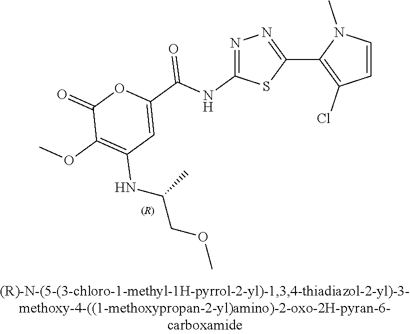 |
| 22 | 129 | 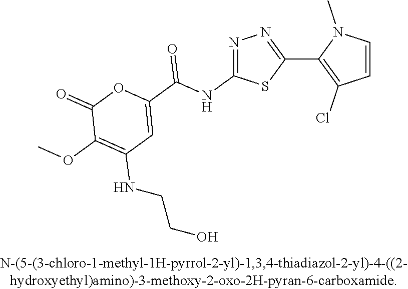 [SMILES: COc1c(NCCO)cc(C(=O)Nc2nnc(-c3c(Cl)ccn3C)s2)oc1=O] |
| 23 | 130 | 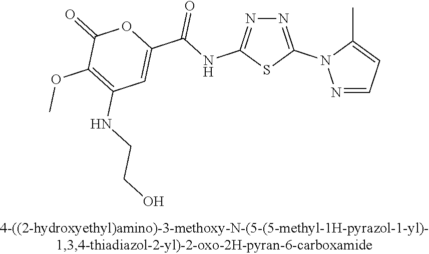 [SMILES: COc1c(NCCO)cc(C(=O)Nc2nnc(-n3nccc3C)s2)oc1=O] |
| 10 | 131 | 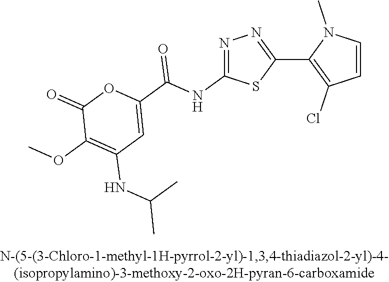 [SMILES: COc1c(NC(C)C)cc(C(=O)Nc2nnc(-c3c(Cl)ccn3C)s2)oc1=O] |
| 24 | 132* | 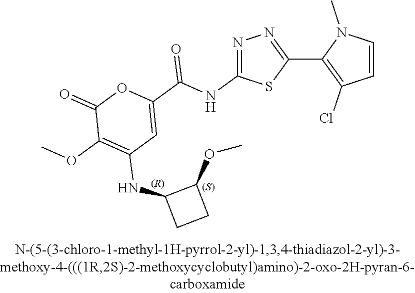 |
| 24 | 133* | 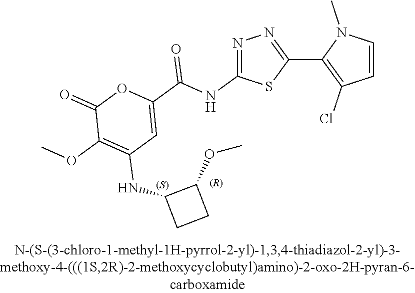 |
| 25 | 134 | 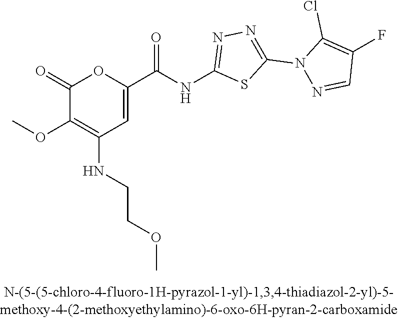 [SMILES: COCCNc1cc(C(=O)Nc2nnc(-n3ncc(F)c3Cl)s2)oc(=O)c1OC] |
| 26 | 135 | 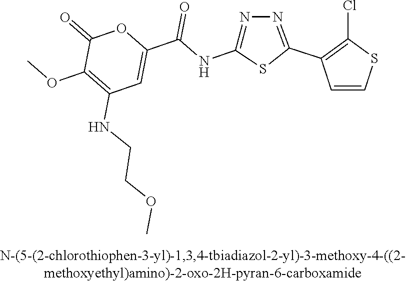 [SMILES: COCCNc1cc(C(=O)Nc2nnc(-c3ccsc3Cl)s2)oc(=O)c1OC] |
| 4 | 136 | 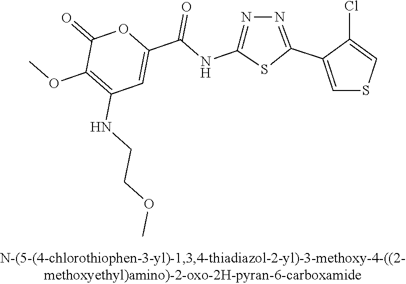 [SMILES: COCCNc1cc(C(=O)Nc2nnc(-c3cscc3Cl)s2)oc(=O)c1OC] |
| 27 | 137 | 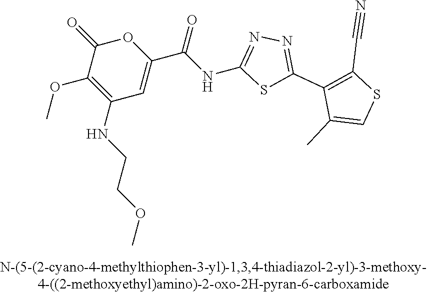 [SMILES: COCCNc1cc(C(=O)Nc2nnc(-c3c(C)csc3C#N)s2)oc(=O)c1OC] |
| 28 | 138 | 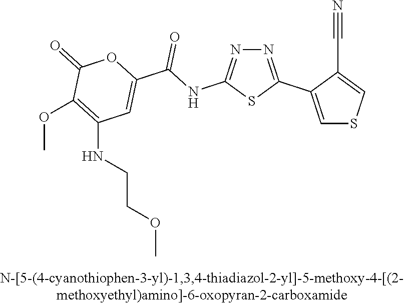 [SMILES: COCCNc1cc(C(=O)Nc2nnc(-c3cscc3C#N)s2)oc(=O)c1OC] |
| 29 | 139 | 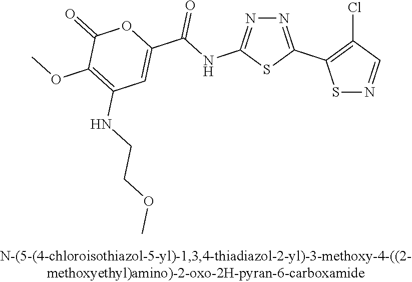 [SMILES: COCCNc1cc(C(=O)Nc2nnc(-c3sncc3Cl)s2)oc(=O)c1OC] |
| 30 | 140 | 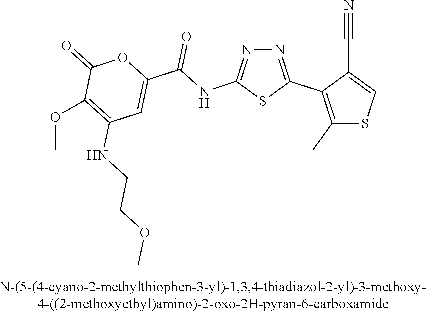 [SMILES: COCCNc1cc(C(=O)Nc2nnc(-c3c(C#N)csc3C)s2)oc(=O)c1OC] |
| 31 | 141 | 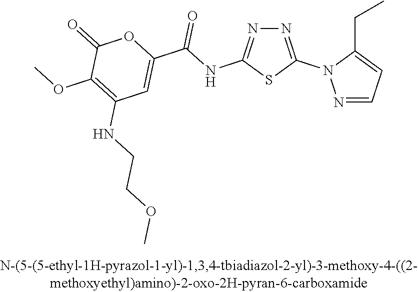 [SMILES: CCc1ccnn1-c1nnc(NC(=O)c2cc(NCCOC)c(OC)c(=O)o2)s1] |
| 32 | 142 | 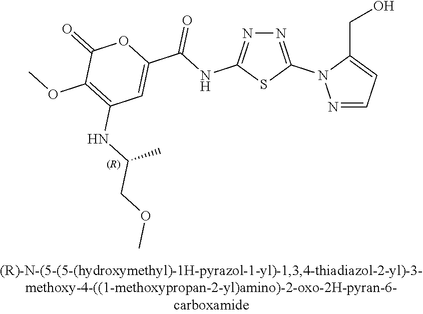 |
| TA | 143 | 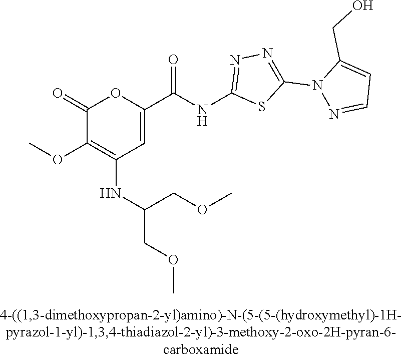 [SMILES: COCC(COC)Nc1cc(C(=O)Nc2nnc(-n3nccc3CO)s2)oc(=O)c1OC] |
| 33 | 144 | 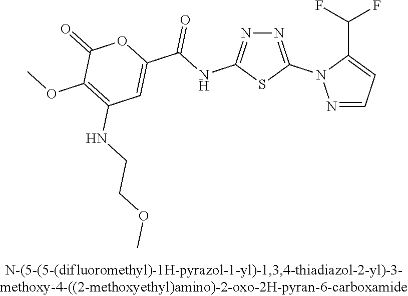 [SMILES: COCCNc1cc(C(=O)Nc2nnc(-n3nccc3C(F)F)s2)oc(=O)c1OC] |
| 34 | 145 | 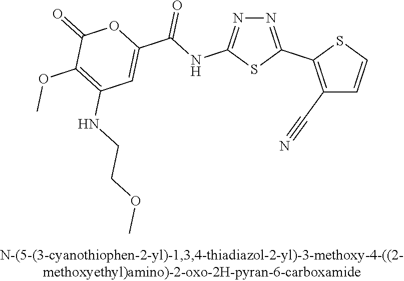 [SMILES: COCCNc1cc(C(=O)Nc2nnc(-c3sccc3C#N)s2)oc(=O)c1OC] |
| TA | 146 | 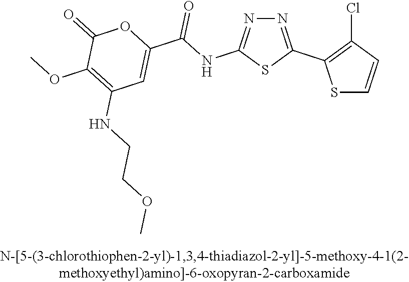 [SMILES: COCCNc1cc(C(=O)Nc2nnc(-c3sccc3Cl)s2)oc(=O)c1OC] |
| TA | 147 | 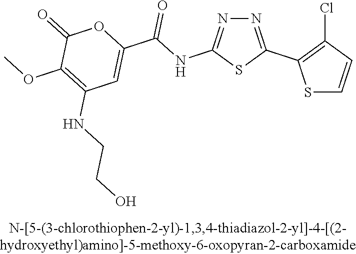 [SMILES: COc1c(NCCO)cc(C(=O)Nc2nnc(-c3sccc3Cl)s2)oc1=O] |
| 35 | 148 | 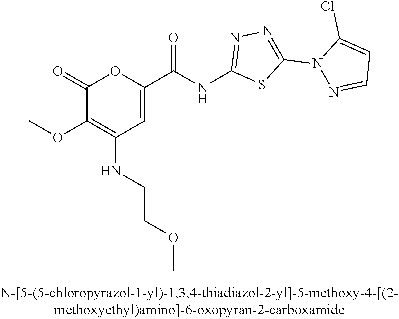 [SMILES: COCCNc1cc(C(=O)Nc2nnc(-n3nccc3Cl)s2)oc(=O)c1OC] |
| 36 | 149 | 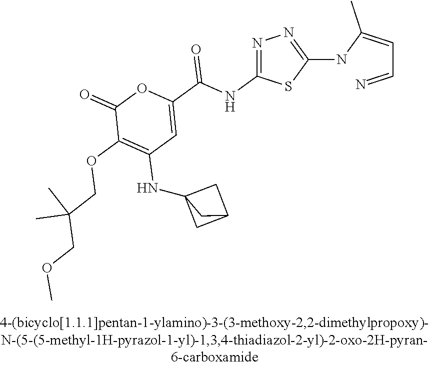 [SMILES: COCC(C)(C)COc1c(NC23CC(C2)C3)cc(C(=O)Nc2nnc(N/C(C)=C\C=N)s2)oc1=O] |
| 37 | 150 | 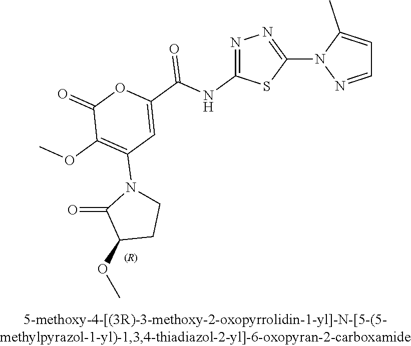 |
| TA | 151 | 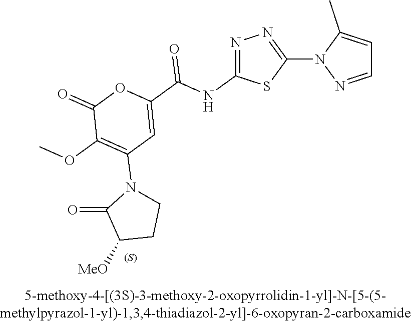 |
| 38 | 152 | 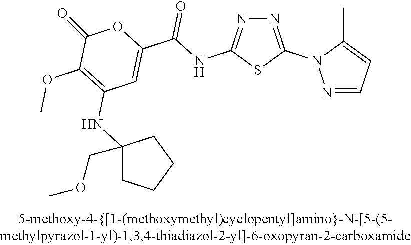 [SMILES: COCC1(Nc2cc(C(=O)Nc3nnc(-n4nccc4C)s3)oc(=O)c2OC)CCCC1] |
| 39 | 153a* | 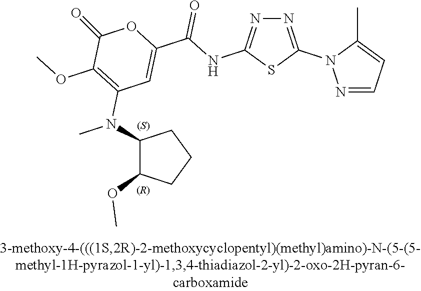 |
| 39 | 153b* | 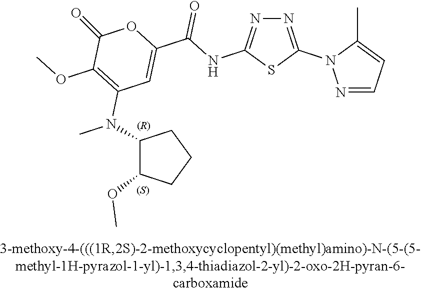 |
| TA | 154 | 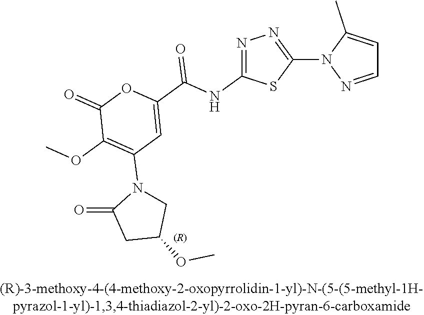 |
| TA | 155 | 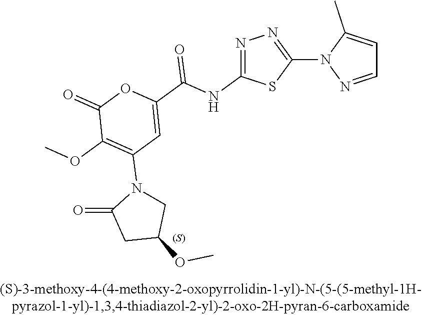 |
| 40 | 156 | 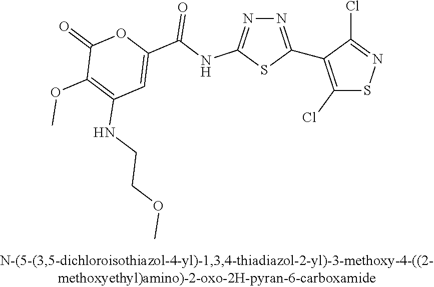 [SMILES: COCCNc1cc(C(=O)Nc2nnc(-c3c(Cl)nsc3Cl)s2)oc(=O)c1OC] |
| 41 | 157 | 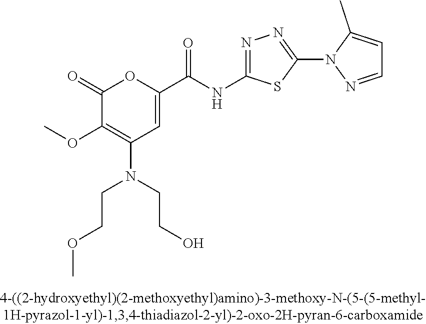 [SMILES: COCCN(CCO)c1cc(C(=O)Nc2nnc(-n3nccc3C)s2)oc(=O)c1OC] |
| 41 | 158 | 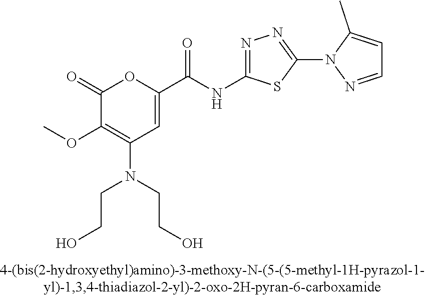 [SMILES: COc1c(N(CCO)CCO)cc(C(=O)Nc2nnc(-n3nccc3C)s2)oc1=O] |
| 42 | 159a* | 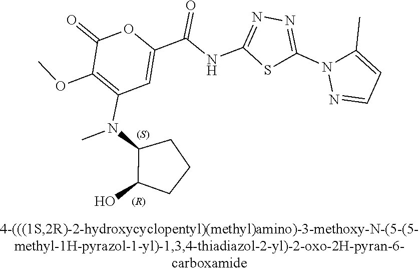 |
| 42 | 159b* | 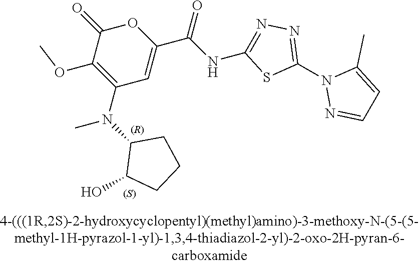 |
| 43 | 160 | 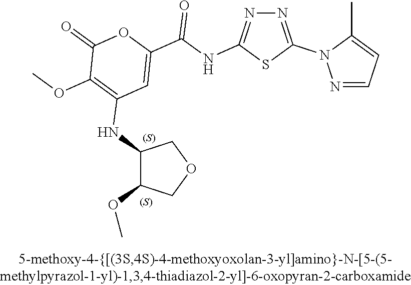 |
| TA | 161 | 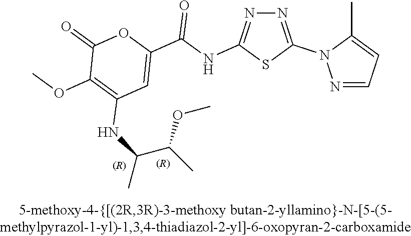 |
| TA | 162 | 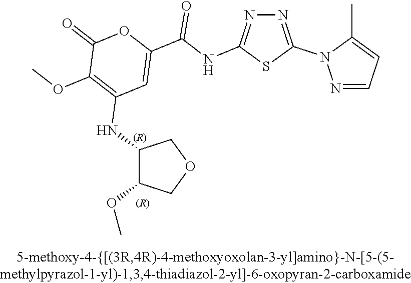 |
| TA | 163 | 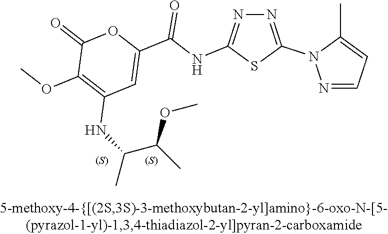 |
| TA | 164a* | 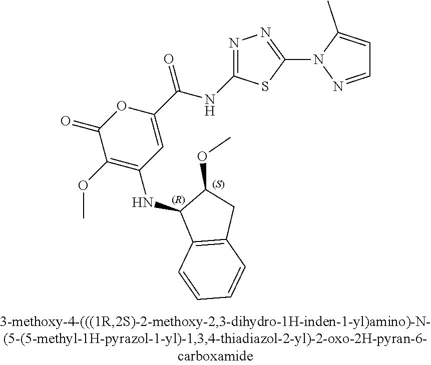 |
| TA | 164b* | 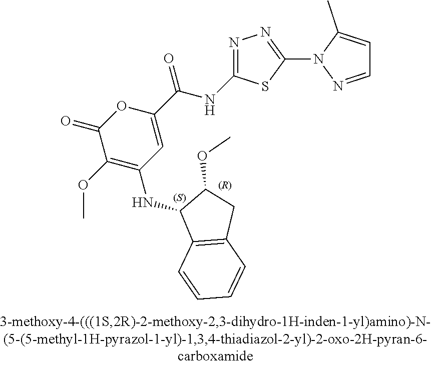 |
| 44 | 165 | 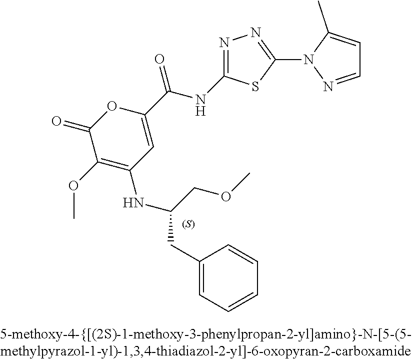 |
| 45 | 166 | 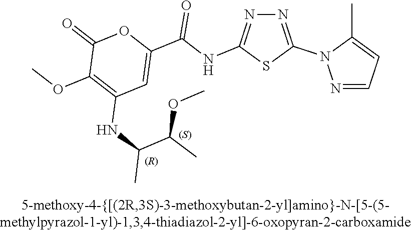 |
| 46 | 167a* | 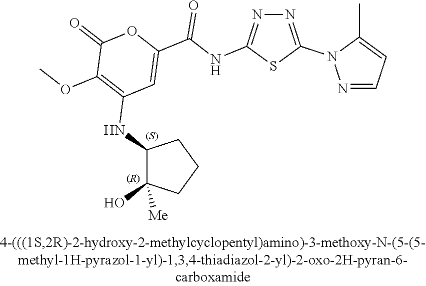 |
| 63 | 56b* | 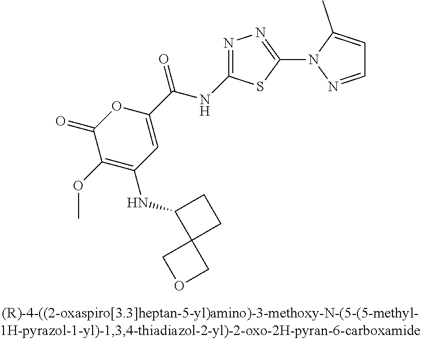 [SMILES: COc1c(N[C@@H]2CCC23COC3)cc(C(=O)Nc2nnc(-n3nccc3C)s2)oc1=O] |
| 46 | 167b* | 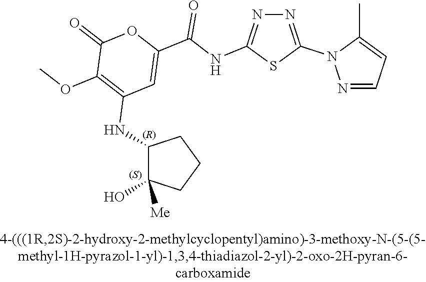 |
| 47 | 168 | 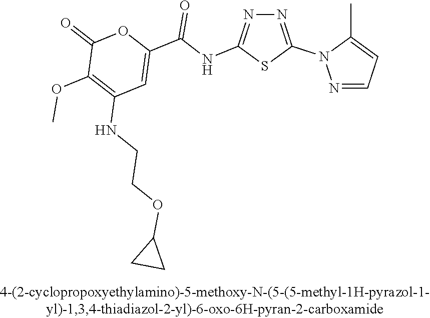 [SMILES: COc1c(NCCOC2CC2)cc(C(=O)Nc2nnc(-n3nccc3C)s2)oc1=O] |
| TA | 169 | 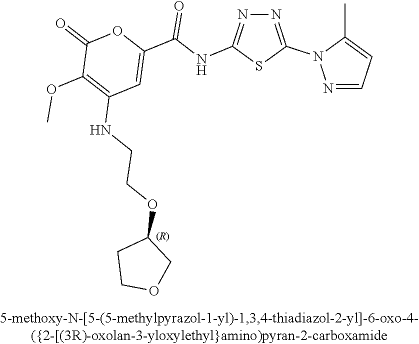 |
| TA | 170 | 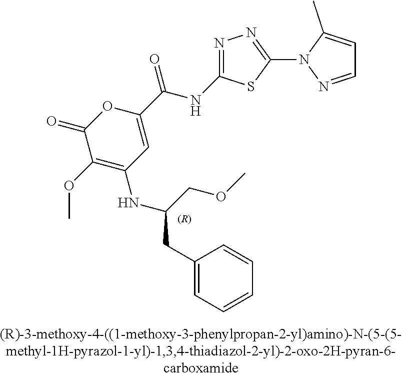 |
| 48 | 171 | 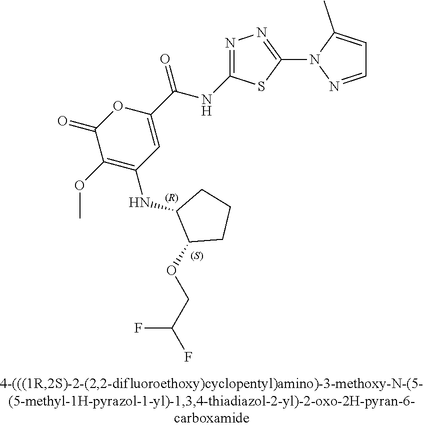 |
| 49 | 172* | 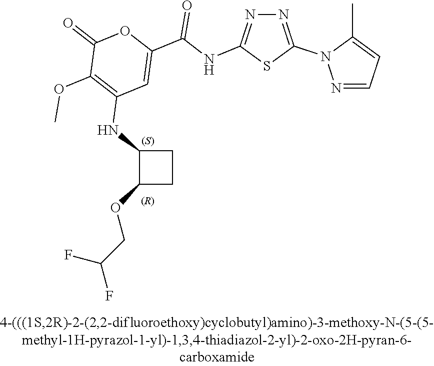 |
| 49 | 173* | 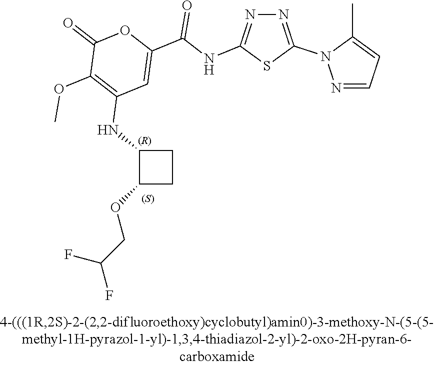 |
| 50 | 174 | 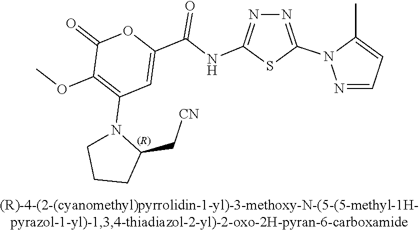 |
| TA | 175 | 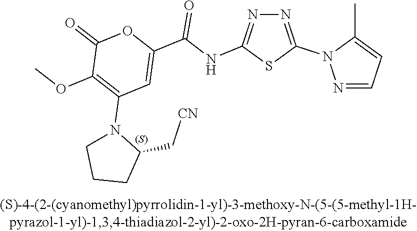 |
| TA | 176 | 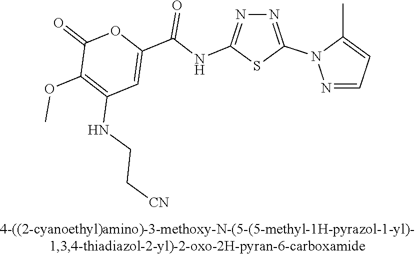 [SMILES: COc1c(NCCC#N)cc(C(=O)Nc2nnc(-n3nccc3C)s2)oc1=O] |
| 51 | 177 | 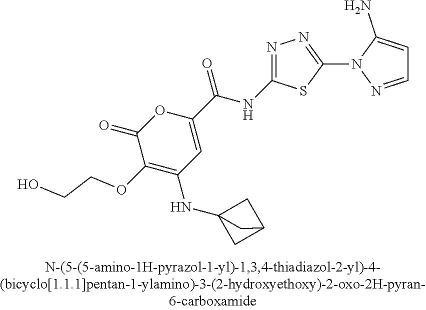 [SMILES: Nc1ccnn1-c1nnc(NC(=O)c2cc(NC34CC(C3)C4)c(OCCO)c(=O)o2)s1] |
| 52 | 178 | 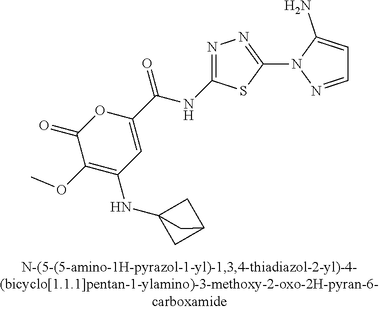 [SMILES: COc1c(NC23CC(C2)C3)cc(C(=O)Nc2nnc(-n3nccc3N)s2)oc1=O] |
| TA | 179 | 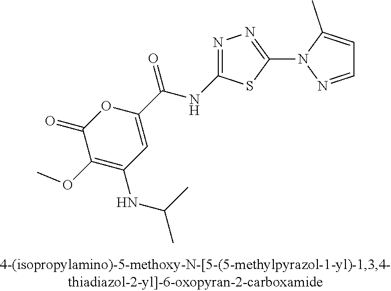 [SMILES: COc1c(NC(C)C)cc(C(=O)Nc2nnc(-n3nccc3C)s2)oc1=O] |
| TA | 180 | 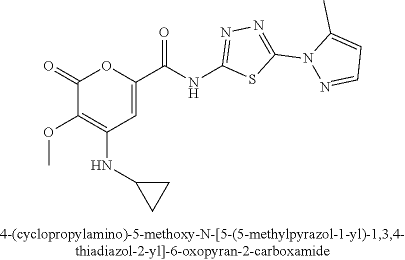 [SMILES: COc1c(NC2CC2)cc(C(=O)Nc2nnc(-n3nccc3C)s2)oc1=O] |
| TA | 181 | 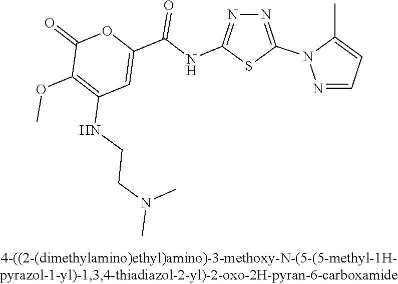 [SMILES: COc1c(NCCN(C)C)cc(C(=O)Nc2nnc(-n3nccc3C)s2)oc1=O] |
| TA | 182 | 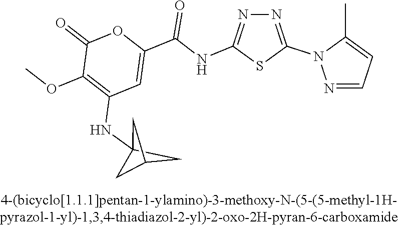 [SMILES: COc1c(NC23CC(C2)C3)cc(C(=O)Nc2nnc(-n3nccc3C)s2)oc1=O] |
| TA | 183 | 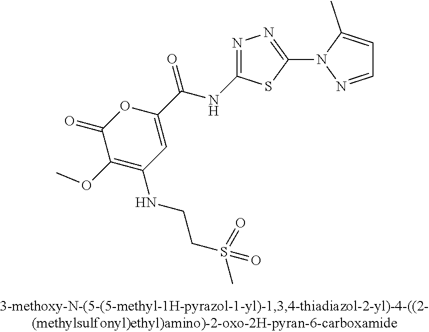 [SMILES: COc1c(NCCS(C)(=O)=O)cc(C(=O)Nc2nnc(-n3nccc3C)s2)oc1=O] |
| TA | 184 | 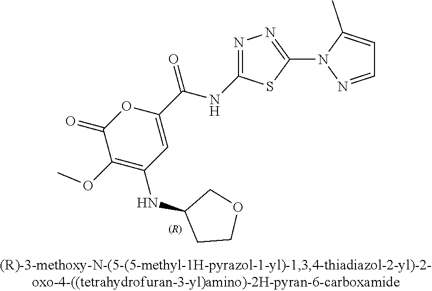 |
| TA | 185 | 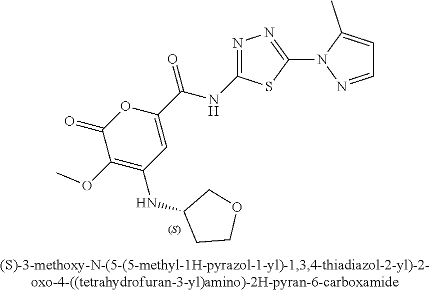 |
| TA | 186 | 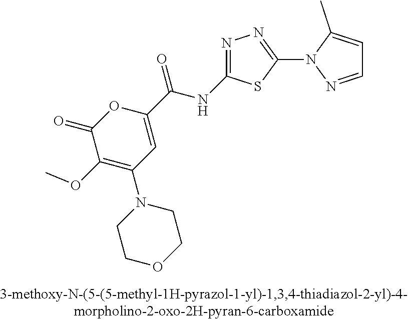 [SMILES: COc1c(N2CCOCC2)cc(C(=O)Nc2nnc(-n3nccc3C)s2)oc1=O] |
| TA | 187 | 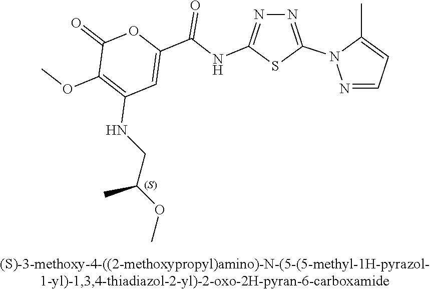 |
| TA | 188a* | 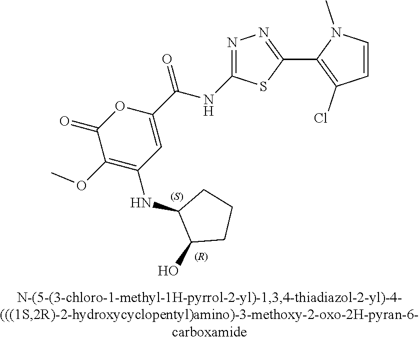 |
| TA | 188b* | 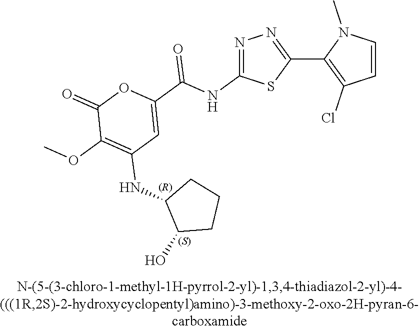 |
| TA | 189a* | 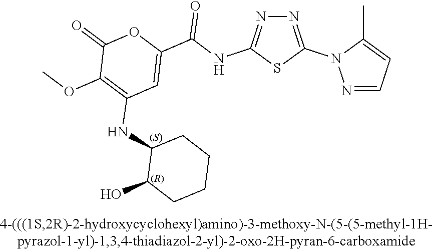 |
| TA | 189b* | 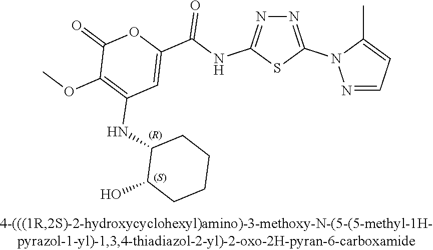 |
| TA | 190 | 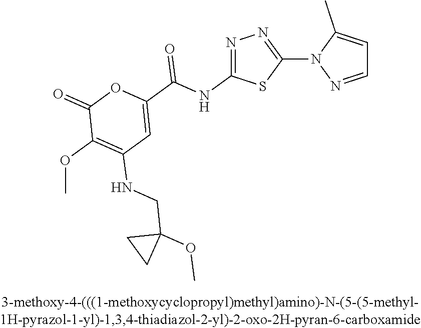 [SMILES: COc1c(NCC2(OC)CC2)cc(C(=O)Nc2nnc(-n3nccc3C)s2)oc1=O] |
| 53 | 191* | 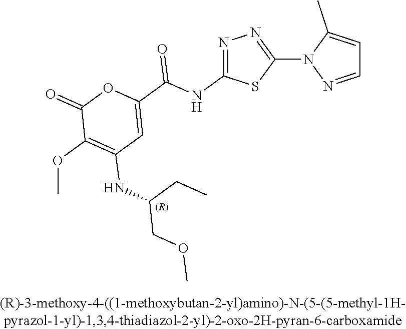 |
| 53 | 192* | 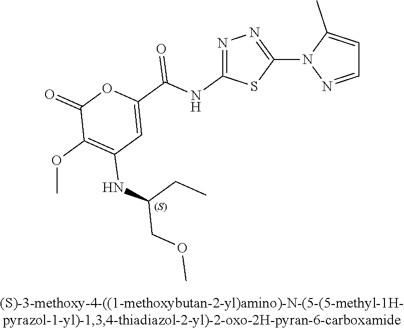 |
| TA | 193 | 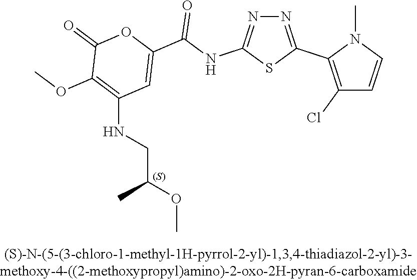 |
| TA | 194 | 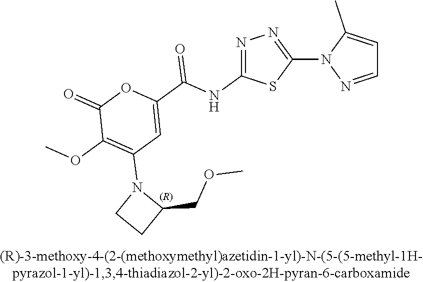 |
| TA | 195 | 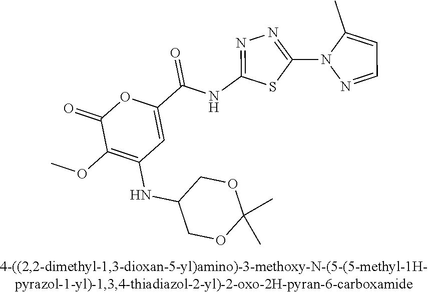 [SMILES: COc1c(NC2COC(C)(C)OC2)cc(C(=O)Nc2nnc(-n3nccc3C)s2)oc1=O] |
| TA | 196 | 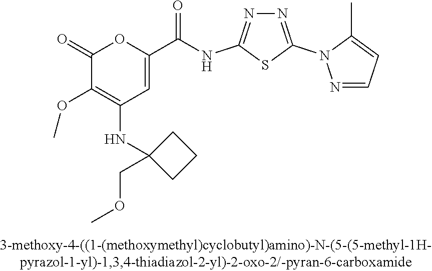 [SMILES: COCC1(Nc2cc(C(=O)Nc3nnc(-n4nccc4C)s3)oc(=O)c2OC)CCC1] |
| TA | 197 | 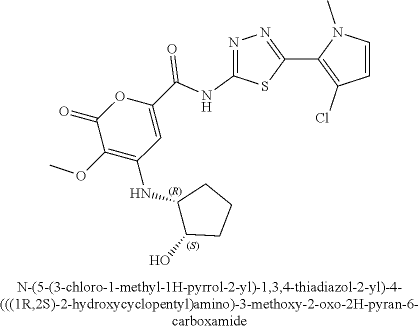 |
| 54 | 198* | 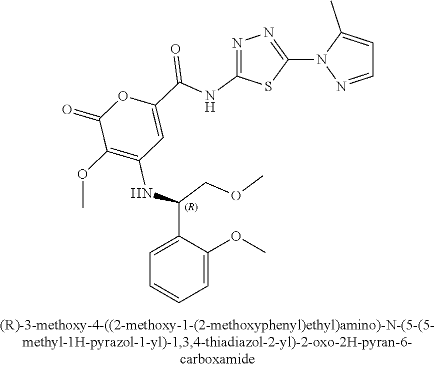 |
| 54 | 199* | 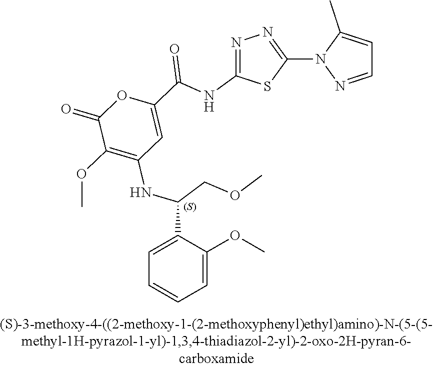 |
| 15 | 200 | 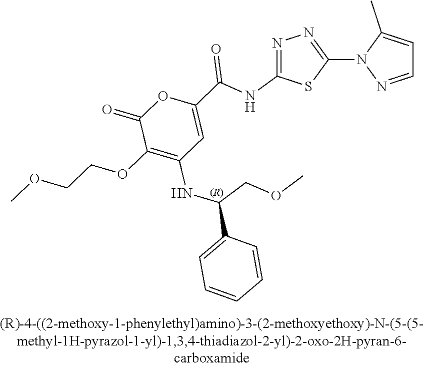 |
| 16 | 201 | 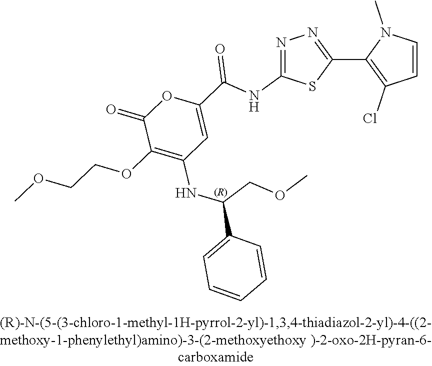 |
| TA | 202 | 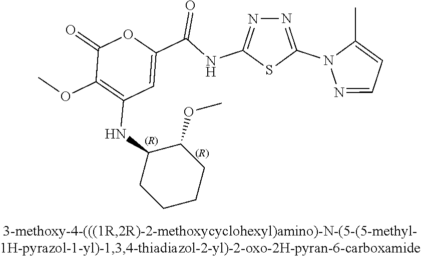 |
| 55 | 203 | 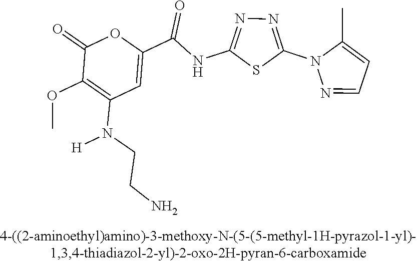 [SMILES: COc1c(NCCN)cc(C(=O)Nc2nnc(-n3nccc3C)s2)oc1=O] |
| TA | 204 | 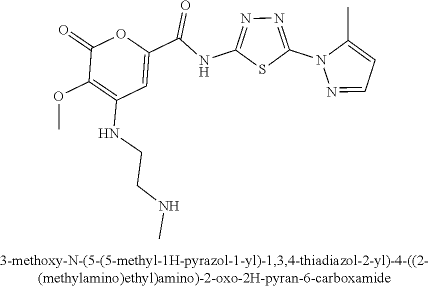 [SMILES: CNCCNc1cc(C(=O)Nc2nnc(-n3nccc3C)s2)oc(=O)c1OC] |
| 56 | 205 | 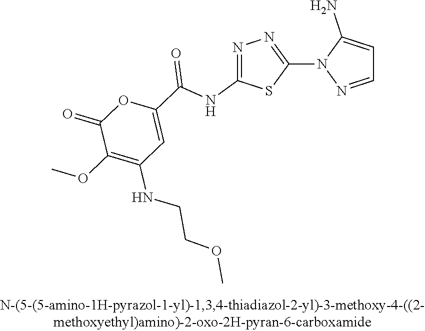 [SMILES: COCCNc1cc(C(=O)Nc2nnc(-n3nccc3N)s2)oc(=O)c1OC] |
| TA | 206 | 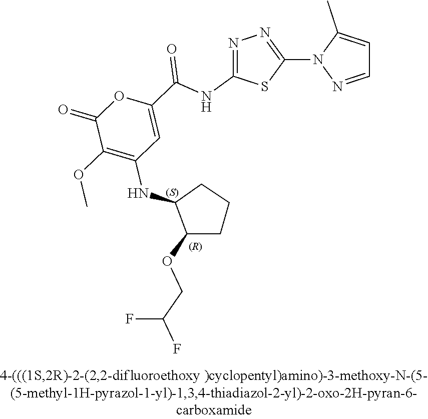 |
| TA | 207 | 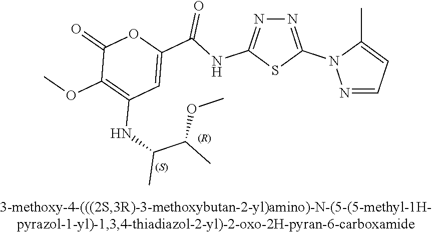 |
| 57 | 208 | 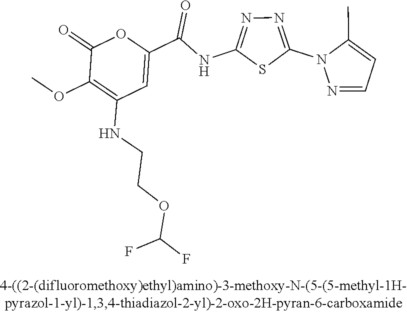 [SMILES: COc1c(NCCOC(F)F)cc(C(=O)Nc2nnc(-n3nccc3C)s2)oc1=O] |
| 58 | 209a* | 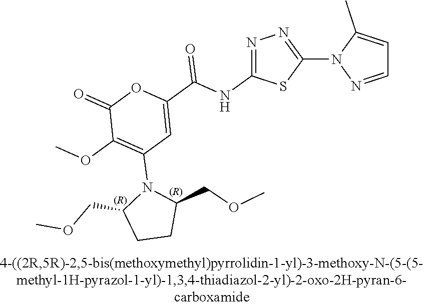 |
| 58 | 209b* | 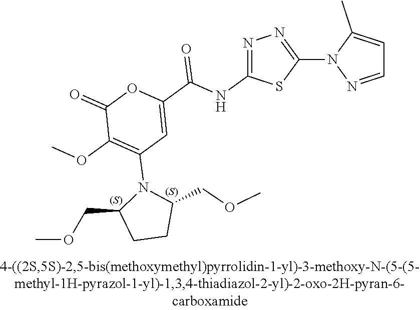 |
| 59 | 210 | 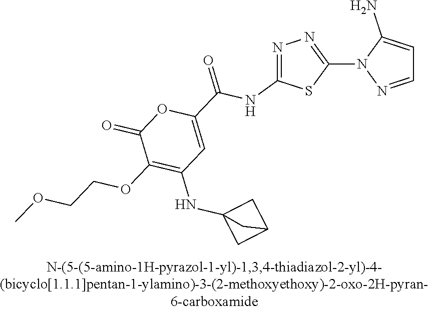 [SMILES: COCCOc1c(NC23CC(C2)C3)cc(C(=O)Nc2nnc(-n3nccc3N)s2)oc1=O] |
| 60 | 211 | 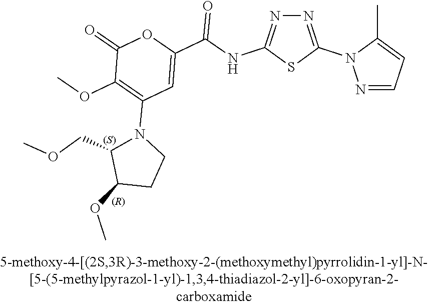 |
| 61 | 212 | 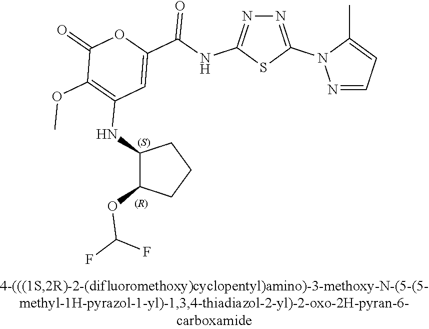 |
| TA | 213 | 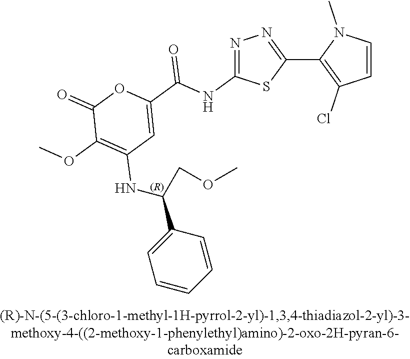 |
| 62 | 214* | 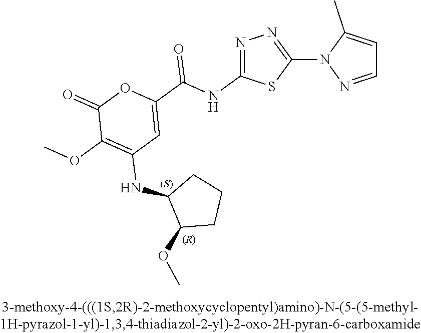 |
| 62 | 215* | 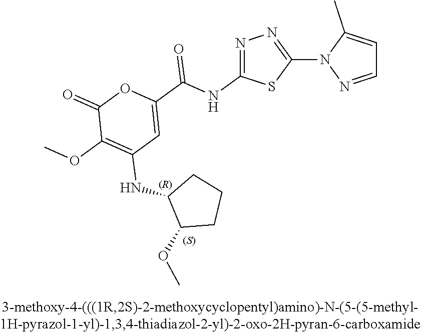 |
| TA | 216 | 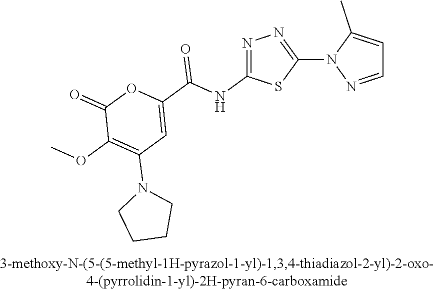 [SMILES: COc1c(N2CCCC2)cc(C(=O)Nc2nnc(-n3nccc3C)s2)oc1=O] |
| TA | 217 | 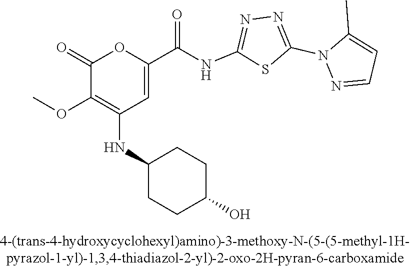 [SMILES: COc1c(N[C@H]2CC[C@H](O)CC2)cc(C(=O)Nc2nnc(-n3nccc3C)s2)oc1=O] |
| TA | 218 | 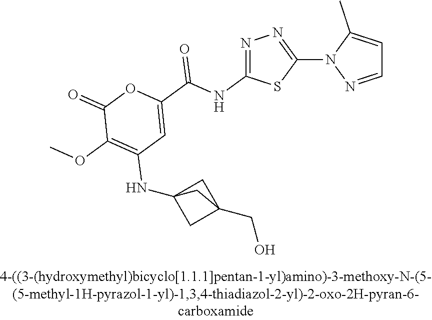 [SMILES: COc1c(NC23CC(CO)(C2)C3)cc(C(=O)Nc2nnc(-n3nccc3C)s2)oc1=O] |
| TA | 219 | 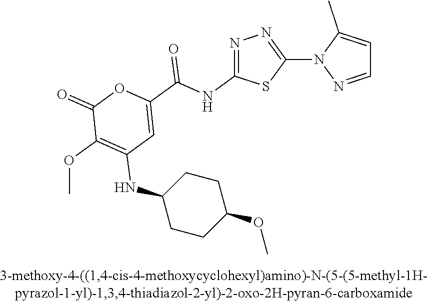 [SMILES: COc1c(N[C@H]2CC[C@@H](OC)CC2)cc(C(=O)Nc2nnc(-n3nccc3C)s2)oc1=O] |
| TA | 220 | 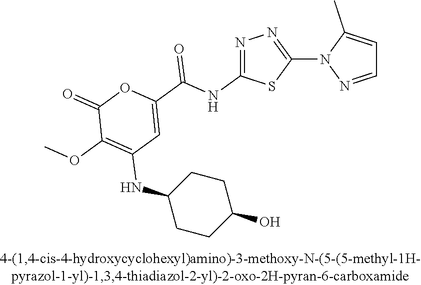 [SMILES: COc1c(N[C@H]2CC[C@@H](O)CC2)cc(C(=O)Nc2nnc(-n3nccc3C)s2)oc1=O] |
| 5 | 177-Ac-OtBu | 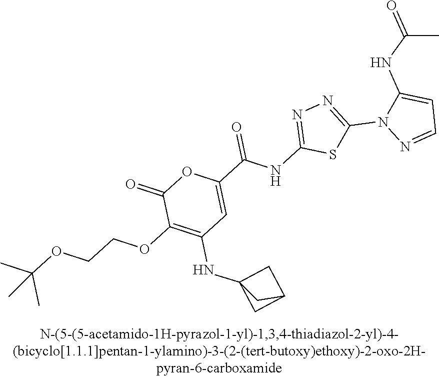 [SMILES: CC(=O)Nc1ccnn1-c1nnc(NC(=O)c2cc(NC34CC(C3)C4)c(OCCOC(C)(C)C)c(=O)o2)s1] |
| 52 | 178-Ac | 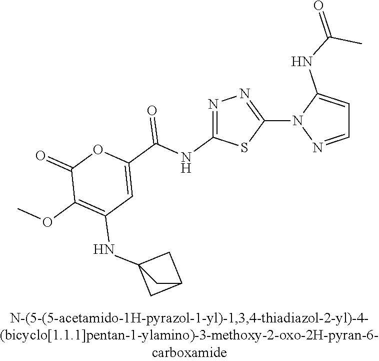 [SMILES: COc1c(NC23CC(C2)C3)cc(C(=O)Nc2nnc(-n3nccc3NC(C)=O)s2)oc1=O] |
| 56 | 205-Ac | 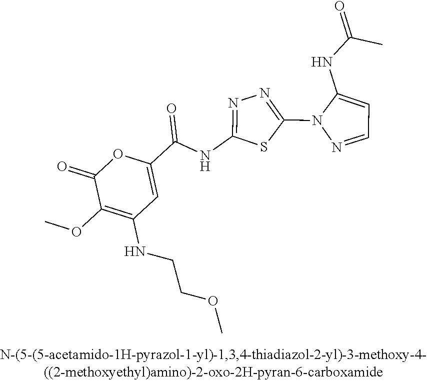 [SMILES: COCCNc1cc(C(=O)Nc2nnc(-n3nccc3NC(C)=O)s2)oc(=O)c1OC] |

TABLE 2
Compounds of Formula (I)

| | | Compound structure and |
|---|---|---|
| Ex | # | name |
| 64 | 221 | 1-(1,3-dimethoxypropan-2-yl)- |
| | | N-(5-(5-methyl-1H-pyrazol-1- |
| | | yl)-1,3,4-thiadiazol-2-yl)-5-oxo- |
| | | 1,2,3,5-tetrahydropyrano[3,4- |
| | | b][1,4]oxazine-7-carboxamide |
| | | 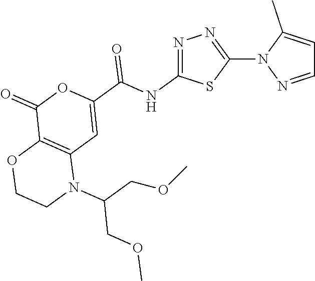 [SMILES: COCC(COC)N1CCOc2c1cc(C(=O)Nc1nnc(-n3nccc3C)s1)oc2=O] |
| TA | 222 | (R)-1-(1-methoxypropan-2-yl)- |
| | | N-(5-(5-methyl-1H-pyrazol-1- |
| | | yl)-1,3,4-thiadiazol-2-yl)-5-oxo- |
| | | 1,2,3,5-tetrahydropyrano[3,4- |
| | | b][1,4]oxazine-7-carboxamide |
| | | 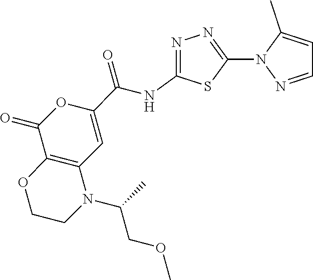 [SMILES: COC[C@@H](C)N1CCOc2c1cc(C(=O)Nc1nnc(-n3nccc3C)s1)oc2=O] |
| TA | 223 | 1-((1R,2S)-2- |
| | | cyanocyclopentyl)-N-(5-(5- |
| | | methyl-1H-pyrazol-1-yl)-1,3,4- |
| | | thiadiazol-2-yl)-5-oxo-1,2,3,5- |
| | | tetrahydropyrano[3,4- |
| | | b][1,4]oxazine-7-carboxamide |
| | | 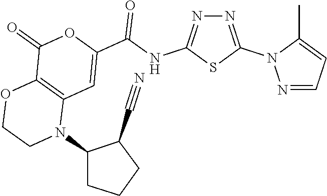 [SMILES: Cc1ccnn1-c1nnc(NC(=O)c2cc3c(c(=O)o2)OCCN3[C@@H]2CCC[C@@H]2C#N)s1] |
| TA | 224 | 1-((1S,2R)-2- |
| | | cyanocyclopentyl)-N-(5-(5- |
| | | methyl-1H-pyrazol-1-yl)-1,3,4- |
| | | thiadiazol-2-yl)-5-oxo-1,2,3,5- |
| | | tetrahydropyrano[3,4- |
| | | b][1,4]oxazine-7-carboxamide |
| | | 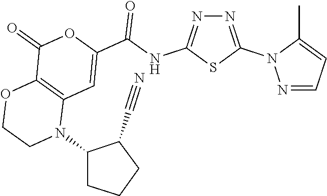 [SMILES: Cc1ccnn1-c1nnc(NC(=O)c2cc3c(c(=O)o2)OCCN3[C@H]2CCC[C@H]2C#N)s1] |
| TA | 225 | (R)-1-(1-cyclopropyl-2- |
| | | methoxyethyl)-N-(5-(5-methyl- |
| | | 1H-pyrazol-1-yl)-1,3,4- |
| | | thiadiazol-2-yl)-5-oxo-1,2,3,5- |
| | | tetrahydropyrano[3,4- |
| | | b][1,4]oxazine-7-carboxamide |
| | | 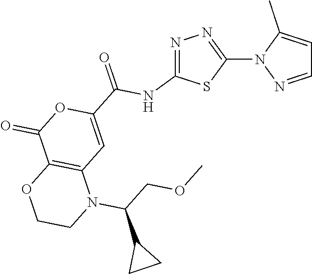 [SMILES: COC[C@@H](C1CC1)N1CCOc2c1cc(C(=O)Nc1nnc(-n3nccc3C)s1)oc2=O] |
| TA | 226 | (S)-1-(1-cyclopropyl-2- |
| | | methoxyethyl)-N-(5-(5-methyl- |
| | | 1H-pyrazol-1-yl)-1,3,4- |
| | | thiadiazol-2-yl)-5-oxo-1,2,3,5- |
| | | tetrahydropyrano[3,4- |
| | | b][1,4]oxazine-7-carboxamide |
| | | 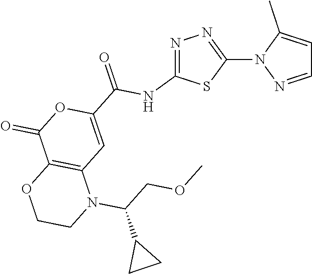 [SMILES: COC[C@H](C1CC1)N1CCOc2c1cc(C(=O)Nc1nnc(-n3nccc3C)s1)oc2=O] |
| TA | 227 | (R)-1-(2-methoxy-1- |
| | | phenylethyl)-N-(5-(5-methyl- |
| | | 1H-pyrazol-1-yl)-1,3,4- |
| | | thiadiazol-2-yl)-5-oxo-1,2,3,5- |
| | | tetrahydropyrano[3,4- |
| | | b][1,4]oxazine-7-carboxamide |
| | | 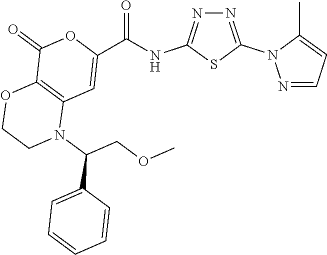 [SMILES: COC[C@@H](c1ccccc1)N1CCOc2c1cc(C(=O)Nc1nnc(-n3nccc3C)s1)oc2=O] |
| TA | 228 | 4-((2-methoxyethyl)amino)-N- |
| | | (5-(5-methyl-1H-pyrazol-1-yl)- |
| | | 1,3,4-thiadiazol-2-yl)-3-(2- |
| | | morpholinoethoxy)-2-oxo-2H- |
| | | pyran-6-carboxamide |
| | | 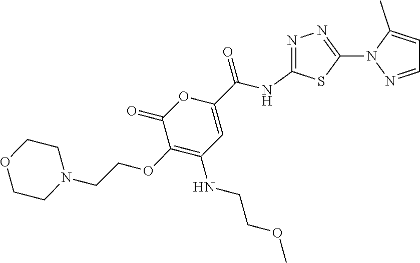 [SMILES: COCCNc1cc(C(=O)Nc2nnc(-n3nccc3C)s2)oc(=O)c1OCCN1CCOCC1] |
| TA | 229 | 4-((2-methoxyethyl)amino)-N- |
| | | (5-(5-methyl-1H-pyrazol-1-yl)- |
| | | 1,3,4-thiadiazol-2-yl)-2-oxo-3- |
| | | (2-thiomorpholinoethoxy)-2H- |
| | | pyran-6-carboxamide |
| | | 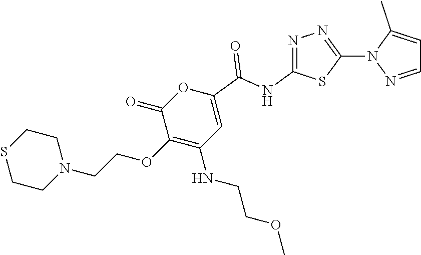 [SMILES: COCCNc1cc(C(=O)Nc2nnc(-n3nccc3C)s2)oc(=O)c1OCCN1CCSCC1] |
| TA | 230 | 3-((1- |
| | | methoxycyclopropyl)methoxy)- |
| | | 4-((2-methoxyethyl)amino)-N- |
| | | (5-(5-methyl-1H-pyrazol-1-yl)- |
| | | 1,3,4-thiadiazol-2-yl)-2-oxo-2H- |
| | | pyran-6-carboxamide |
| | | 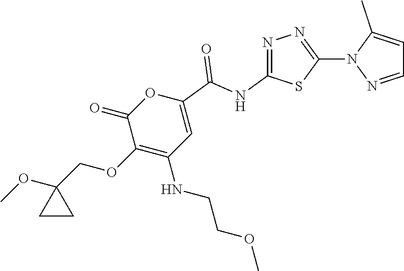 [SMILES: COCCNc1cc(C(=O)Nc2nnc(-n3nccc3C)s2)oc(=O)c1OCC1(OC)CC1] |
| TA | 231A | (S)-4-((2-methoxyethyl)amino)- |
| | | N-(5-(5-methyl-1H-pyrazol-1- |
| | | yl)-1,3,4-thiadiazol-2-yl)-2-oxo- |
| | | 3-((tetrahydrofuran-3-yl)oxy)- |
| | | 2H-pyran-6-carboxamide |
| | | 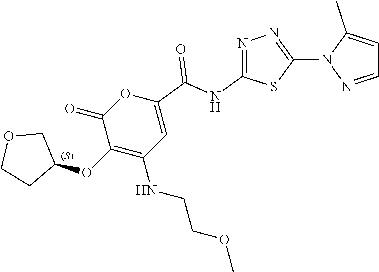 |
| TA | 231B | (R)-4-((2-methoxyethyl)amino)- |
| | | N-(5-(5-methyl-1H-pyrazol-1- |
| | | yl)-1,3,4-thiadiazol-2-yl)-2-oxo- |
| | | 3-((tetrahydrofuran-3-yl)oxy)- |
| | | 2H-pyran-6-carboxamide |
| | | 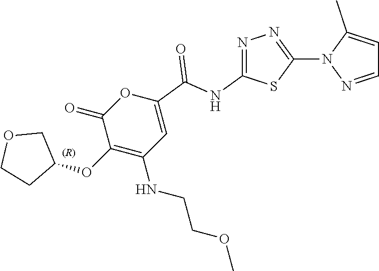 |

In some embodiments, the compound is Compound 3*, Compound 4*, Compound 10, Compound 21*, Compound 22*, Compound 67a*, Compound 67b*, Compound 73, Compound 74, Compound 77, Compound 83, Compound 107a*, Compound 107b*, Compound 108a*, Compound 108b*, Compound 114, Compound 121, Compound 127, Compound 161, Compound 182, Compound 16, Compound 197, Compound 213, or a pharmaceutically acceptable salt of any of the foregoing.

### ii. Pharmaceutical Compositions

In some aspects, the present disclosure provides a pharmaceutical composition comprising a compound of Formula (I), or a pharmaceutically acceptable salt thereof, and a pharmaceutically acceptable carrier. Exemplary pharmaceutical acceptable carriers include excipients, diluents, and surfactants.

In some embodiments, the compound of the present disclosure, or pharmaceutical composition comprising same, can be administered in an amount effective to treat a disorder in a subject.

Administration can be accomplished via any mode of administration. Exemplary modes include systemic or local administration such as oral, nasal, parenteral, transdermal, subcutaneous, vaginal, buccal, rectal or topical administration modes.

Depending on the intended mode of administration, the disclosed compounds and compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

Likewise, the disclosed compounds and compositions can also be administered by intravenous (both bolus and infusion), intraperitoneal, subcutaneous or intramuscular in a form suitable for these types of administration. For example, parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Illustrative pharmaceutical compositions may be tablets or gelatin capsules comprising a compound of the present disclosure, or a pharmaceutically acceptable salt thereof, and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant. e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, or PEG200.

### iii. Methods of Treatment

In some aspects, the present disclosure provides a method of treating a disease or disorder disclosed herein in a subject in need thereof, comprising administering to the subject an amount of a compound of the present disclosure, or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject an effective amount of a compound of the present disclosure, or a pharmaceutically acceptable salt thereof, or a pharmaceutical composition of the present disclosure.

In some aspects, the present disclosure provides a method of modulating cGAS activity (e.g., in vitro or in vivo), comprising contacting a cell with an effective amount of a compound of the present disclosure or a pharmaceutically acceptable salt thereof.

In some embodiments, the disease or disorder is associated with implicated cGAS activity. In some embodiments, the disease or disorder is a disease or disorder in which cGAS activity is implicated.

In some aspects, the present disclosure provides a compound of the present disclosure, or a pharmaceutically acceptable salt thereof, for use in modulating cGAS activity (e.g., in vitro or in vivo).

In some aspects, the present disclosure provides a compound of the present disclosure, or a pharmaceutically acceptable salt thereof, for use in treating a disease or disorder disclosed herein.

In some aspects, the present disclosure provides use of a compound of the present disclosure, or a pharmaceutically acceptable salt thereof, in the manufacture of a medicament for modulating cGAS activity (e.g., in vitro or in vivo).

In some aspects, the present disclosure provides use of a compound of the present disclosure, or a pharmaceutically acceptable salt thereof, in the manufacture of a medicament for treating a disease or disorder disclosed herein.

In some aspects, the present disclosure provides use of a compound of the present disclosure, or a pharmaceutically acceptable salt thereof, in the manufacture of a medicament for treating a disease or disorder disclosed herein.

The present disclosure provides compounds that function as modulators of cGAS activity.

In some embodiments, modulation is inhibition.

In some embodiments, the disease or disorder is inflammation, an autoimmune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an cGAS-related disease in a subject that has been determined to carry a germline or somatic non-silent mutation in cGAS.

In some aspects, the disease or disorder is cancer. In some embodiments, the cancer is bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, pancreatic cancer, penile cancer, testicular germ cell cancer, thymoma carcinoma, thymic carcinoma, lung cancer, ovarian cancer, or prostate cancer.

In some aspects, the disease or disorder is a central nervous system disorder. In certain embodiments, the central nervous system is Parkinson's disease, Alzheimer's disease, traumatic brain injury, spinal cord injury, amyotrophic lateral sclerosis (ALS), multiple sclerosis, ataxia telangiectasia, or age-related macular degeneration.

In some aspects, the disease or disorder is kidney disease. In certain embodiments, the kidney disease is acute kidney disease, chronic kidney disease, or a rare kidney disease. In certain embodiments, the chronic kidney disease is diabetic nephropathy.

In some aspects, the disease or disorder is a skin disease. In certain embodiments, the skin disease is psoriasis, hidradenitis suppurativa (HS), or atopic dermatitis.

In some aspects, the disease or disorder is a rheumatic disease. In certain embodiments, the rheumatic disease is dermatomyositis, Still's disease, or juvenile idiopathic arthritis. In some aspects, the disease or disorder is a liver disease. In certain embodiments, the liver disease is nonalcoholic steatohepatitis (NASH).

In some aspects, the disease or disorder is a cardiovascular disease. In certain embodiments, the cardiovascular disease is cardiomyopathy, atherosclerosis or peripheral artery disease (PAD).

In some embodiments, the disease or disorder is a metabolic disease. In certain embodiments, the metabolic disease is obesity-induced insulin-resistance.

In some aspects, the disease or disorder is a cGAS-related disease in a subject that has been determined to carry a germline or somatic non-silent mutation in cGAS.

In some embodiments, the disease or disorder is an inflammatory, allergic or autoimmune disease such as systemic lupus erythematosus (SLE), cutaneous lupus erythematosus (CLE), Chilblain lupus, psoriasis, insulin-dependent diabetes mellitus (IDDM), scleroderma, Aicardi Goutières syndrome, dermatomyositis, systemic sclerosis, inflammatory bowel diseases, multiple sclerosis, rheumatoid arthritis, chronic kidney disease, or Sjogren's syndrome (SS).

In some embodiments, the disease or disorder is inflammation of any tissue or organ of the body, including musculoskeletal inflammation, vascular inflammation, neural inflammation, digestive system inflammation, ocular inflammation, inflammation of the reproductive system, and other inflammation.

In some embodiments, musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic). Ocular inflammation refers to inflammation of any structure of the eye, including the eye lids. Examples of ocular inflammation include blepharitis, blepharochalasis, conjunctivitis, dacryoadenitis, keratitis, keratoconjunctivitis sicca (dry eye), scleritis, trichiasis, and uveitis. Examples of inflammation of the nervous system include encephalitis. Guillain-Barre syndrome, meningitis, neuromyotonia, narcolepsy, multiple sclerosis, myelitis and schizophrenia.

Examples of inflammation of the vasculature or lymphatic system include arthrosclerosis, arthritis, phlebitis, vasculitis, and lymphangitis.

Examples of inflammatory conditions of the digestive system include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system include cervicitis, chorioamnionitis, endometritis, epididymitis, omphalitis, oophoritis, orchitis, salpingitis, tubo-ovarian abscess, urethritis, vaginitis, vulvitis, and vulvodynia.

In some embodiments, the disease or disorder is an autoimmune conditions having an inflammatory component. Such conditions include systemic lupus erythematosus, cutaneous lupus erythematosus, acute disseminated alopecia universalise, Bechet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, Goodpasture's syndrome, Grave's disease, Guillain-Barre syndrome, Hashimoto's disease, Henoch-Schonlein purpura, Kawasaki's disease, microscopic colitis, microscopic polyarteritis, mixed connective tissue disease, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, Aicardi Goutières syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

In some embodiments, the disease or disorder is a T-cell mediated hypersensitivity diseases having an inflammatory component. Such conditions include contact hypersensitivity, contact dermatitis (including that due to poison ivy), urticaria, skin allergies, respiratory allergies (hay fever, allergic rhinitis) and gluten-sensitive enteropathy (Celiac disease).

In some embodiments, other inflammatory conditions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sezary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukemia and lymphomas in adults, acute leukemia of childhood, regional enteritis, autoimmune vasculitis, multiple sclerosis, chronic obstructive pulmonary disease, solid organ transplant rejection, sepsis.

### iv. Methods of Preparation

Compounds of Formula (I) may be synthesized following General Schemes 1-10, as provided below. The Examples further described non-limiting examples of this general syntheses.

For example, as depicted in General Scheme 1,3-hydroxy-2-oxo-2H-pyran-6-carboxylic acid of Formula (A), or salt thereof, may be protected as the alkyl ester of Formula (B), or salt thereof, wherein R^{a} is C₁₋₆ alkyl or C₁₋₆ haloalkyl, followed by halogenation at the C4 position to provide a compound of Formula (C), or salt thereof, wherein X is Cl, Br, or I. Hydroxyl protection with group R¹, as defined herein, may provide a compound of Formula (D), or salt thereof. Deprotection of the alkyl ester of Formula (D), or salt thereof, may provide a carboxylic acid compound of Formula (D), or salt thereof, wherein R¹ is hydrogen.

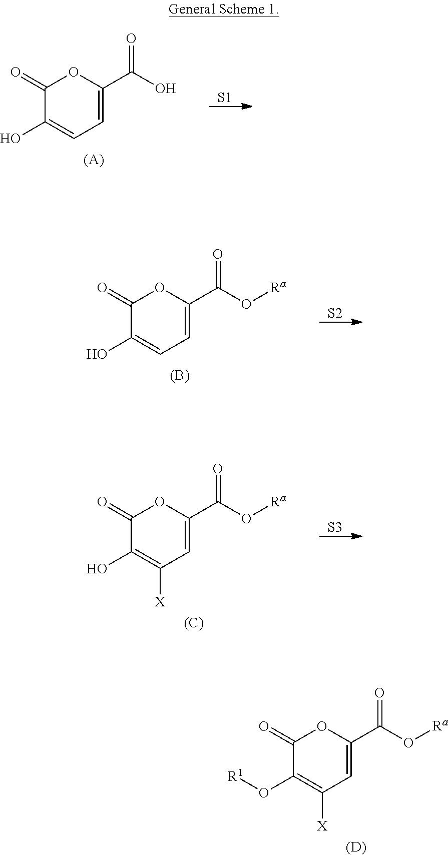

As depicted in General Scheme 2, reacting a hydrazine carbothioamide of Formula (G), or salt thereof, with a carboxylic acid containing compound of Formula (F-1), or a cyano containing compound of Formula (F-2), or salts thereof, wherein R⁴ and m are as defined herein, may provide a 1,3,4-thiadiazol-2-amine of Formula (H-1), or salt thereof. Alternatively, as depicted in General Scheme 3, amine compounds of Formula (H-2), wherein the nitrogen atom of the heteroaryl Ring A is directly linked to the thiadiazole moiety and wherein R⁴ and m are as defined herein, may be prepared by coupling a 5-halo-1,3,4-thiadiazol-2-amine of Formula (M), or salt thereof, wherein Y is Cl, Br, or I, with an amine of Formula (L), or salt thereof.

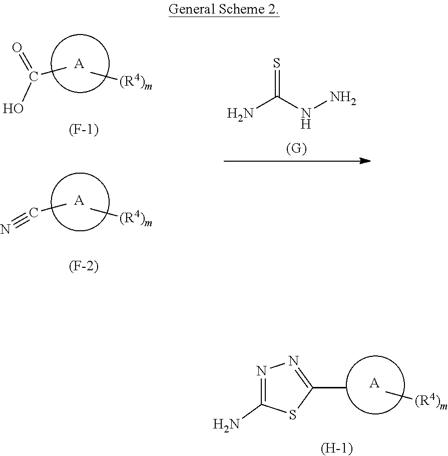

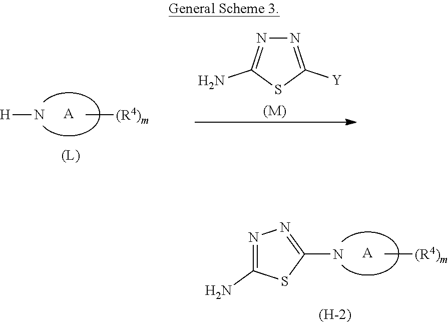

As depicted in General Scheme 4, cross-coupling of an amine of Formula (K), or salt thereof, with an alkyl ester of Formula (D), or salt thereof, wherein R^{a} is C₁₋₆ alkyl or C₁₋₆ haloalkyl, may provide an amine compound of Formula (N), or salt thereof. The amine compound of Formula (N), or salt thereof, may then be deprotected to provide an amine compound of Formula (N), or salt thereof, wherein R^{a} is hydrogen.

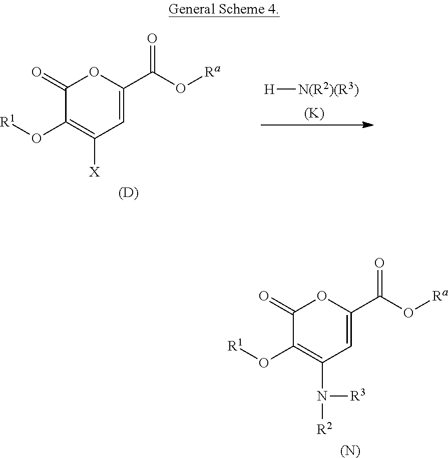

The above-described compounds of Formula (D) and (N), or salts thereof, wherein R^{a} is hydrogen, C₁₋₆ alkyl or C₁₋₆ haloalkyl, and amine compounds of Formula (H-1) or (H-2), or salts thereof, each may be used as intermediates in preparing compounds of Formula (I), or salts thereof.

For example, as depicted in General Scheme 5, peptide coupling the amine of Formula (H-1), or salt thereof, with the compound of Formula (D), or salt thereof, wherein R^{a} is hydrogen, C₁₋₆ alkyl or C₁₋₆ haloalkyl, may provide an amide compound of Formula (J-1), or salt thereof. The amide compound of Formula (J-1), or salt thereof, may then be cross-coupled with an amine of Formula (K), or salt thereof, to provide a compound of Formula (I), or salt thereof.

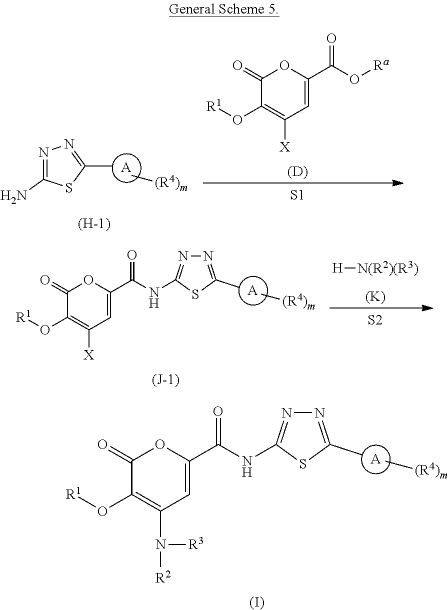

Alternatively, as depicted in General Scheme 6, peptide coupling of the amine of Formula (H-2), or salt thereof, with the compound of Formula (D), or salt thereof, wherein R^{a} is hydrogen, C₁₋₆ alkyl or C₁₋₆ haloalkyl, may provide an amide compound of Formula (J-2), or salt thereof. The amide compound of Formula (J-2), or salt thereof, may then be cross-coupled with an amine of Formula (K), or salt thereof, to provide a compound of Formula (I″″), or salt thereof, wherein the nitrogen atom of the heteroaryl Ring A is directly linked to the thiadiazole moiety.

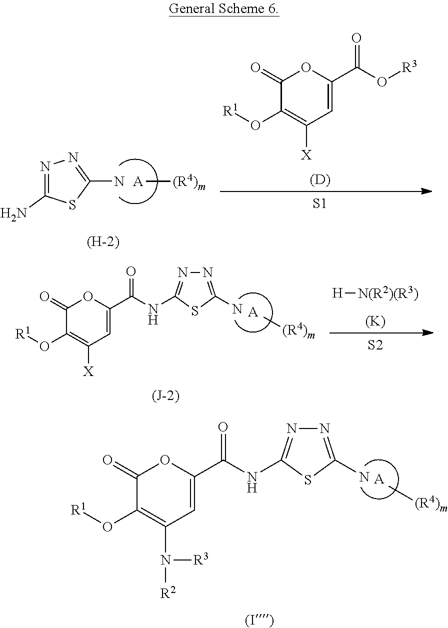

Compounds of Formula (J-1) and (J-2), and salts thereof, are also referred to herein as the “halo-pyrone reagent”s, and compounds of Formula (K), and salts thereof, are also referred to herein as the “amine reagent”s.

In other embodiments, such as depicted in General Scheme 7, peptide coupling of the amine of Formula (H-1), or salt thereof, with a compound of Formula (N), or salt thereof, wherein R^{a} is hydrogen, C₁₋₆ alkyl or C₁₋₆ haloalkyl, may provide a compound of Formula (I), or salt thereof.

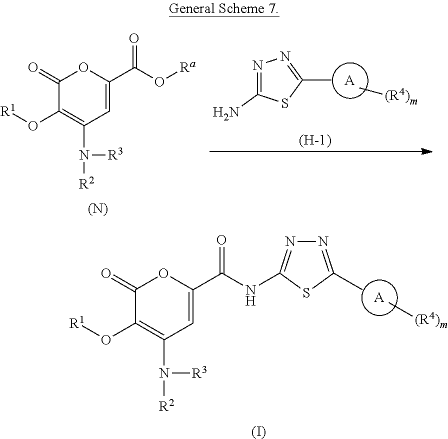

In yet other embodiments, such as depicted in General Scheme 8, peptide coupling of the amine of Formula (H-2), or salt thereof, with a compound of Formula (N), or salt thereof, wherein R^{a} is hydrogen, C₁₋₆ alkyl or C₁₋₆ haloalkyl, may provide a compound of Formula (I″″), or salt thereof, wherein the nitrogen atom of the heteroaryl Ring A is directly linked to the thiadiazole moiety.

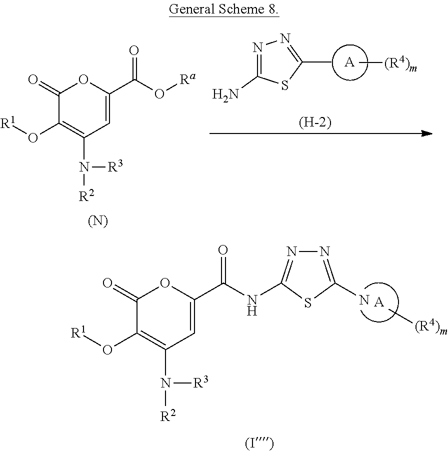

Compounds of Formula (N), and salts thereof, are also referred to as the “amino-pyrone reagent”s, and compounds of Formula (H-1) and (H-2), and salts thereof, are also referred to as the “ADT amine reagent”s.

In still yet other embodiments, such as depicted in General Schemes 9 and 10, wherein R¹ is the group —CH₂CH₂—OH and R² is hydrogen, a bicyclic compound of Formula (I-BC-a) and (I-BC-b) may be formed from conversion of the terminal —OH of R¹ to a leaving group (LG), as defined herein, followed by cyclization. In certain embodiments, the leaving group is a sulfonyl substituted hydroxyl group, such as —O-tosyl, —O-mesyl, or O-besyl.

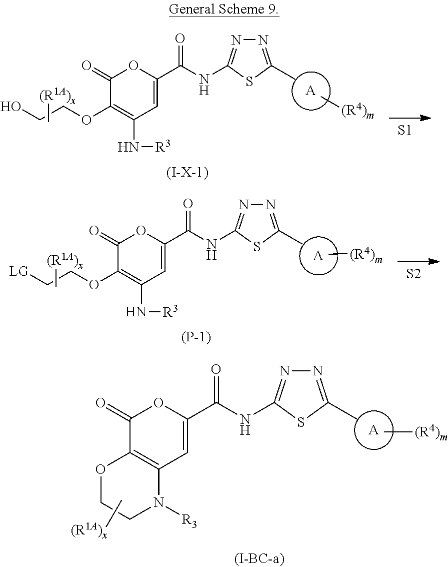

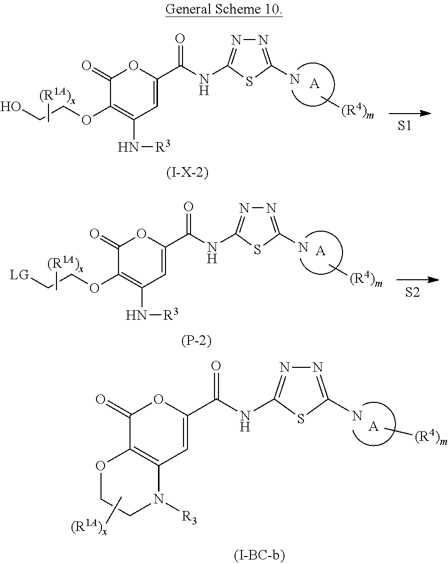

### v. Biological Assays

Compounds designed, selected and/or optimized by methods described above, once produced, can be characterized using a variety of assays known to those skilled in the art to determine whether the compounds have biological activity. For example, the compounds described herein can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the compounds described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, as well as assays for determining hcGAS potency and inhibitory activity, unbound clearance, solubility, and permeability.

In some embodiments, the compounds of the instant disclosure may be tested for their human-cGAS (h-cGAS) inhibitory activity using known procedures, such as the methodology reported in Lama et al., Nature Communications (2019) 10:2261 (2019). See also Examples, Biological Assay Methods.

In some embodiments, the compounds of the instant disclosure may be tested for unbound clearance (Clu) following known procedures, such as described in Miller et al., J. Med Chem. (2020) 63:12156-12170. For example, unbound clearance (Clu) may be calculated by dividing total clearance (‘CL’ in ml/min/kg) as measured in blood or plasma by the unbound fraction in plasma (fu).

In some embodiments, the solubility of compounds of the instant disclosure may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two hour incubation period, followed by filtration, and reported in uM by LC-MS/MS. Thermodynamic solubility in physiologically relevant media may be measured by LC-MS/MS, after a twenty-four hour incubation, followed by filtration, and reported in mg/mL.

In some embodiments, the permeability of compounds of the instant disclosure may be determined following known procedures, such as described in Wang et al. J Mass Spectrom. (2000) 35:71-76. For example, permeability across cell membranes may be measured using either Caco-2 or MDCK-MDR1 cell lines in Transwell plates, after measuring the compound in both apical and basolateral chambers, and reported as an apparent permeability Papp A-B in 10⁻⁶ cm/s.

### vi. Additional Embodiments

Embodiment 1. A compound of Formula (I):

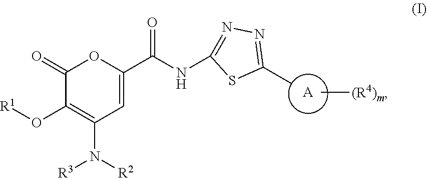

or pharmaceutically acceptable salts thereof, wherein:
Ring A is a 5-membered monocyclic heteroaryl;
R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄ haloalkyl, wherein the alkyl and haloalkyl are independently substituted with 0, 1, 2, 3, or 4 R^{1D}; each R^{1C} is independently hydrogen, C₁-C₃alkyl, C₁-C₃ haloalkyl, or —OR^{1F}; each R^{1D} is independently halogen, —OR^{1F}, or —N(R^{1F})₂; and each R^{1F} is independently hydrogen, C₁-C₃alkyl, or C₁-C₃haloalkyl;
L₃ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O;
R² is hydrogen or C₁-C₆ alkyl substituted with 0, 1, 2, 3, or 4 R^{2A}, and each R^{2A} is independently halogen, —OR^{2B}, or —N(R²)₂, wherein each R¹ is independently hydrogen, C₁-C₃ alkyl, or C₁-C₃ haloalkyl;
or R¹ and R² are joined, with the atoms to which they are attached, to form a 6- or 7-membered heterocyclyl independently substituted with 0, 1, 2, 3, or 4 R^{1A};
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂ or -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₆ alkyl or C₁-C₃ haloalkyl;
each R^{3D} is independently halogen, —OR^{3E}, —CN, C₁-C₃ alkyl, or C₁-C₃ haloalkyl;
each R^{3E} is independently hydrogen, C₁-C₄ alkyl, or C₁-C₄ haloalkyl;
each R⁴ is independently halogen, —CN, -L₂-OR^{4A}, -L₂-N(R^{4B})₂, C₁-C₆ alkyl, or C₁-C₆ haloalkyl, wherein each R^{4A} and R^{4B} are independently hydrogen, C₁-C₃ alkyl, C₁-C₃ haloalkyl, or —C(═O)R^{4C}, wherein R¹ is C₁-C₆ alkyl or C₁-C₃ haloalkyl;
each L₁ and L₂ is independently a bond, C₁-C₃ alkylene, or C₁-C₃ haloalkylene; and
m is 0, 1 or 2.

Embodiment 2. The compound of Embodiment 1, or a pharmaceutically acceptable salt thereof, wherein:
Ring A is a 5-membered monocyclic heteroaryl;
R¹ is C₁-C₆ alkyl, C₂-C₆ alkenyl, or C₂-C₆ alkynyl, wherein the alkyl, alkenyl, and alkynyl are independently substituted with 0, 1, 2, 3, or 4 R^{1A}; each R^{1A} is independently halogen, —OR^{3B}, or —N(R^{1B})₂; and each R^{1B} is independently hydrogen, C₁-C₃ alkyl or C₁-C₃haloalkyl;
R² is hydrogen or C₁-C₆ alkyl substituted with 0, 1, 2, 3, or 4 R^{2A}, and each R^{2A} is independently halogen, —OR^{2B}, or —N(R^{2B})₂, wherein each R^{2B} is independently hydrogen, C₁-C₃ alkyl, or C₁-C₃ haloalkyl;
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₁-C₃ alkynyl, halogen, ═O, -L₁-CN, -L₁-SOR^{3C}, -L₁-SO₂R^{3C}, -L₁-SR^{3B}, -L₁-OR^{3B}, -L₁-N(RM)₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, C₁-C₃ alkyl, or C₁-C₃ haloalkyl;
each R^{3E} is independently hydrogen, C₁-C₃ alkyl, or C₁-C₃ haloalkyl;
each R⁴ is independently halogen, —CN, -L₂-OR^{4A}, -L₂-N(R⁴)₂, C₁-C₆ alkyl, or C₁-C₆ haloalkyl, wherein each R^{4A} and R^{4B} are independently hydrogen, C₁-C₃ alkyl, or C₁-C₃ haloalkyl;
each L₁ and L₂ is independently a bond, C₁-C₃ alkylene, or C₁-C₃ haloalkylene; and
m is 0, 1 or 2.

Embodiment 3. The compound of Embodiment 1 or 2, or a pharmaceutically acceptable salt thereof, wherein the amino moiety

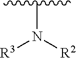

is a group of formula (i-a), (ii-a), or (iii-a):

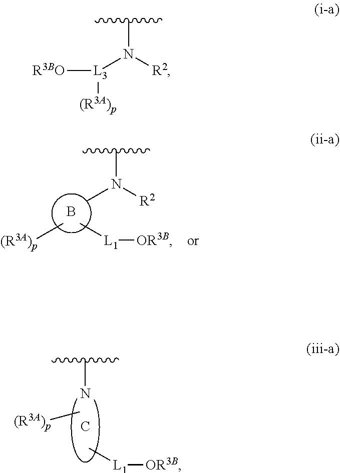

wherein:
L₃ is C₁-C₁₀ alkylene, C₂-C₁₀ alkenylene, or C₂-C₁₀ alkynylene;
Ring B is a C₃-C₁₀ carbocyclyl or 4- to 10-membered heterocyclyl;
Ring C is a 5- to 10-membered heterocyclyl; and
p is 0, 1, 2, or 3.

Embodiment 4. The compound of Embodiment 1 or 2, wherein the compound is of Formula (I′):

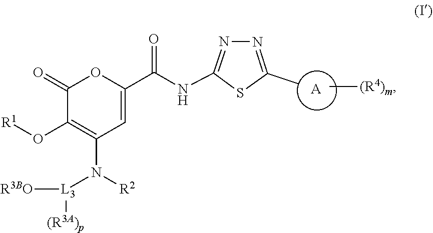

or a pharmaceutically acceptable salt thereof, wherein L₃ is C₁-C₁₀ alkylene, C₂-C₁₀ alkenylene, or C₁-C₁₀ alkynylene, and p is 0, 1, 2, or 3.

Embodiment 5. The compound of Embodiment 1 or 2, wherein the compound is of Formula (I″):

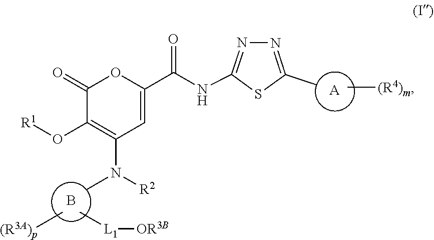

or a pharmaceutically acceptable salt thereof, wherein Ring B is C₃-C₁₀ carbocyclyl or 4- to 10-membered heterocyclyl, and p is 0, 1, 2, or 3.

Embodiment 6. The compound of Embodiment 1 or 2, wherein the compound is of Formula (I′″):

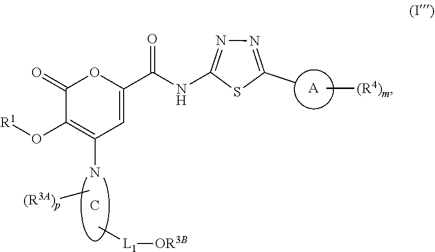

or a pharmaceutically acceptable salt thereof, wherein Ring C is a 5- to 10-membered heterocyclyl, and p is 0, 1, 2, or 3.

Embodiment 7. The compound of Embodiment 1 or 2, wherein the compound is of Formula (I″″):

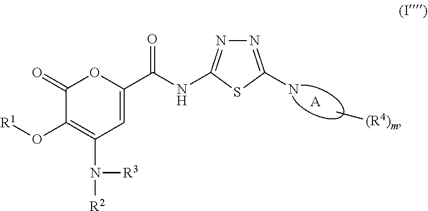

or a pharmaceutically acceptable salt thereof, wherein the nitrogen atom of the heteroaryl Ring A is directly linked to the thiadiazole moiety.

Embodiment 8. The compound of any one of Embodiments 1-7, or a pharmaceutically acceptable salt thereof, wherein R¹ is C₁-C₆ alkyl substituted with 0, 1, 2, 3, or 4 R^{1A}.

Embodiment 9. The compound of Embodiment 8, or a pharmaceutically acceptable salt thereof, wherein R¹ is —CH₃, —CH₂—C(CH₃)₂—CH₂OCH₃, —CH₂CH₂OH, —CH₂CH₂OCH₃,

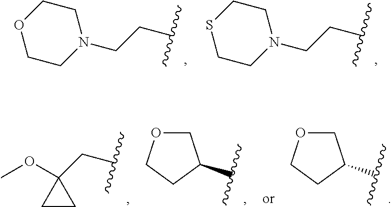

Embodiment 10. The compound of any one of Embodiments 1-9, or a pharmaceutically acceptable salt thereof, wherein R² is hydrogen.

Embodiment 11. The compound of any one of Embodiments 1-10, or a pharmaceutically acceptable salt thereof, wherein R³ is C₁-C₁₀ alkyl substituted with 0, 1, 2, 3, or 4 R^{3A}.

Embodiment 12. The compound of any one of Embodiments 1-10, or a pharmaceutically acceptable salt thereof, wherein R³ is C₃-C₁₀ carbocyclyl substituted with 0, 1, 2, 3, or 4 R^{3A}.

Embodiment 13. The compound of any one of Embodiments 1-10, or a pharmaceutically acceptable salt thereof, wherein R³ is 4- to 10-membered heterocyclyl substituted with 0, 1, 2, 3, or 4 R^{3A}.

Embodiment 14. The compound of any one of Embodiments 1-10, or a pharmaceutically acceptable salt thereof, wherein R² and R³ are joined, with the atom to which they are attached, to form a 4- to 10-membered heterocyclyl independently substituted with 0, 1, 2, 3, or 4 R^{3A}.

Embodiment 15. The compound of any one of Embodiments 1-10, or a pharmaceutically acceptable salt thereof, wherein R² and R³ are joined, with the atom to which they are attached, to form a 4- to 10-membered heterocyclyl independently substituted with 0, 1, 2, 3, or 4 R^{3A}.

Embodiment 16. The compound of any one of Embodiments 1-15, or a pharmaceutically acceptable salt thereof, wherein

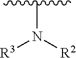

is selected from the group consisting of:

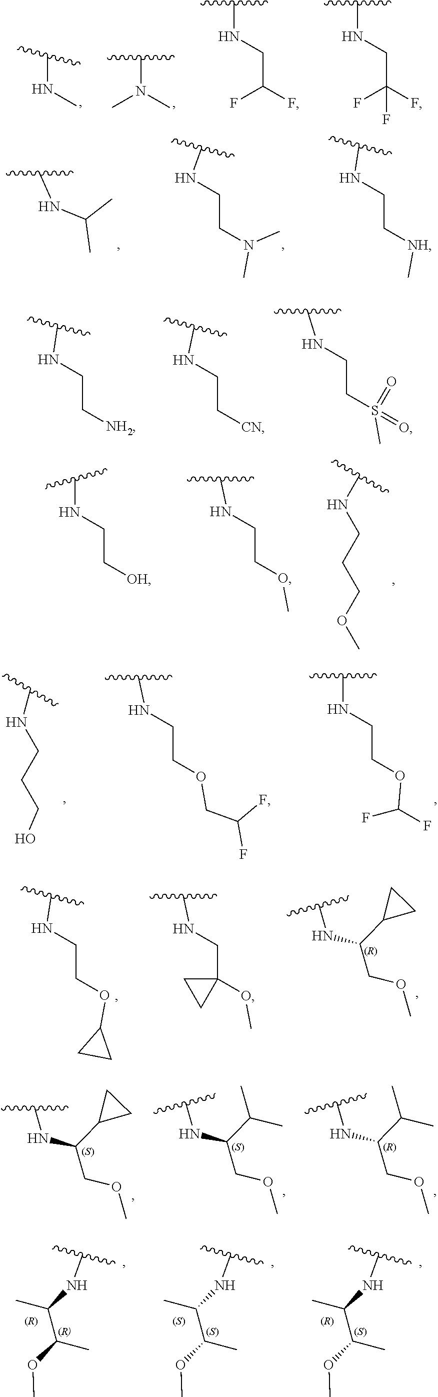

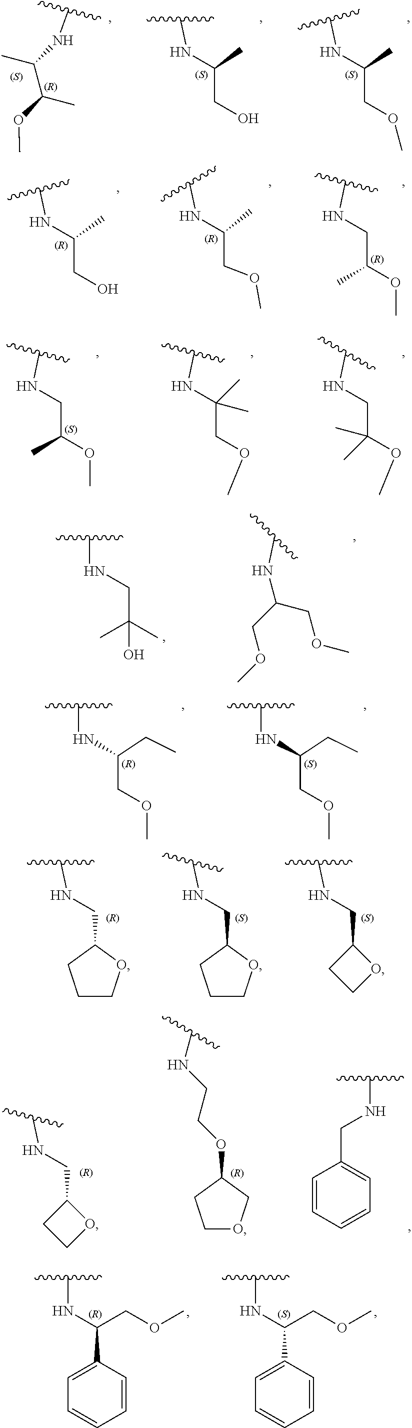

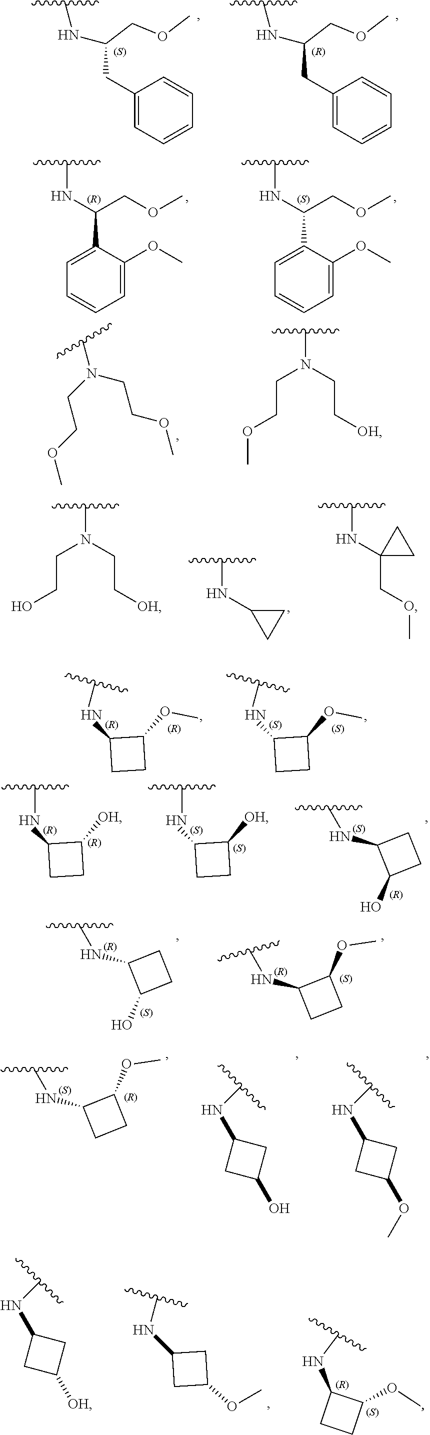

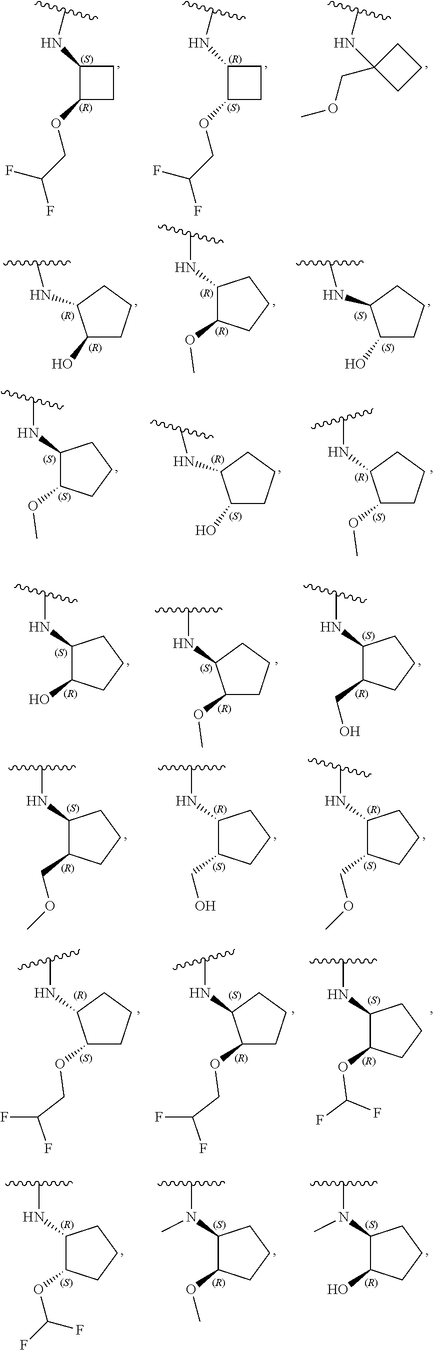

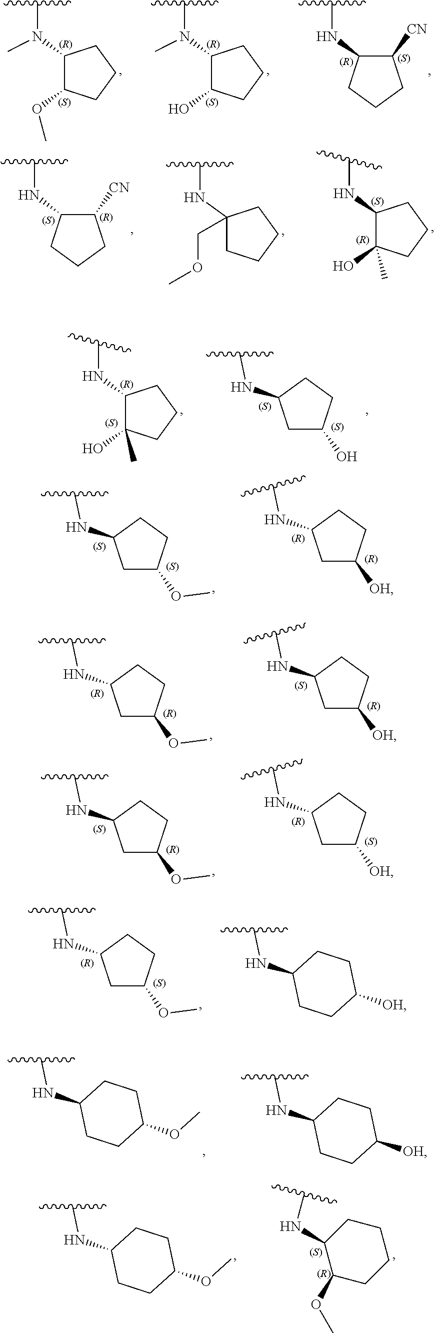

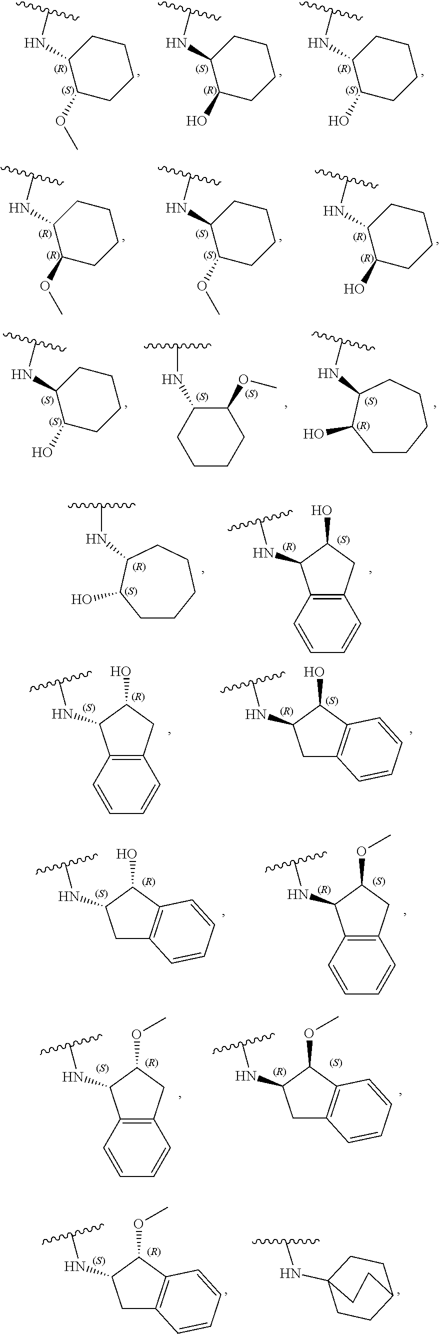

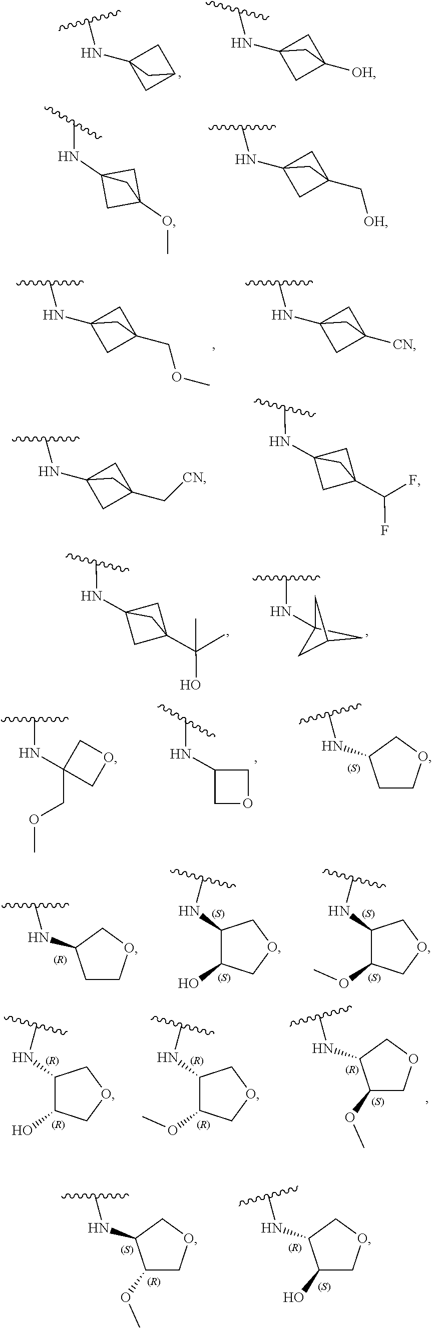

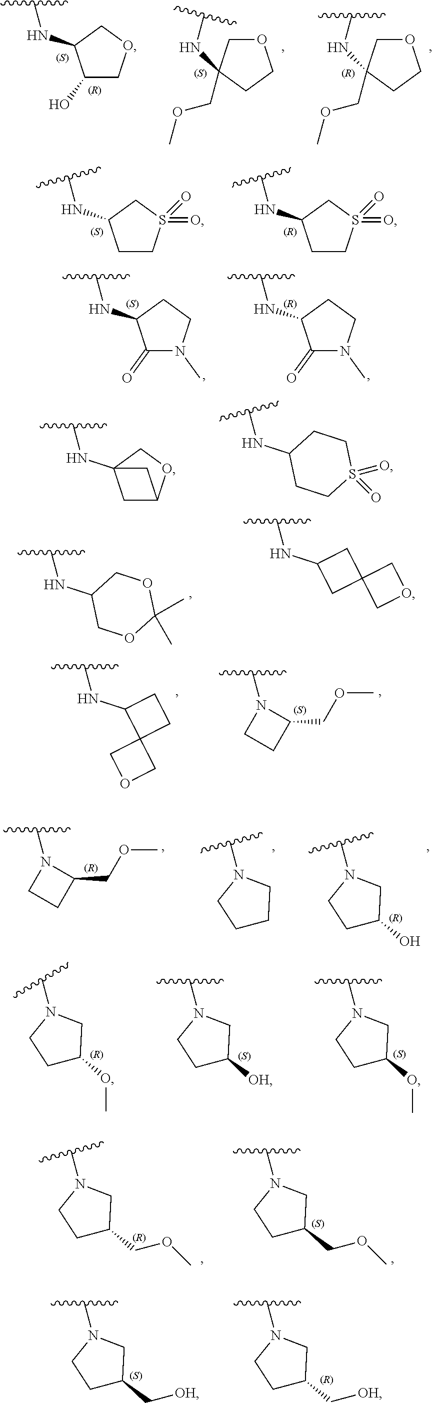

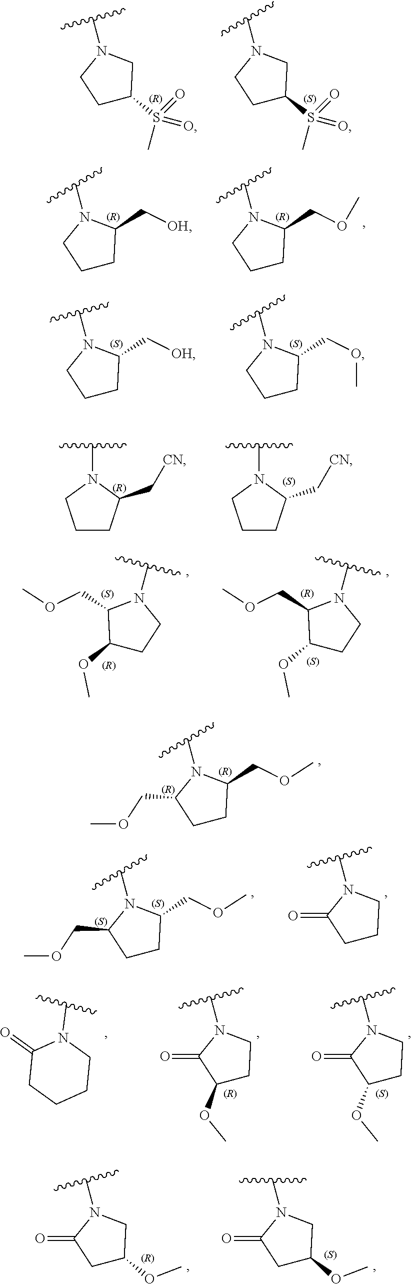

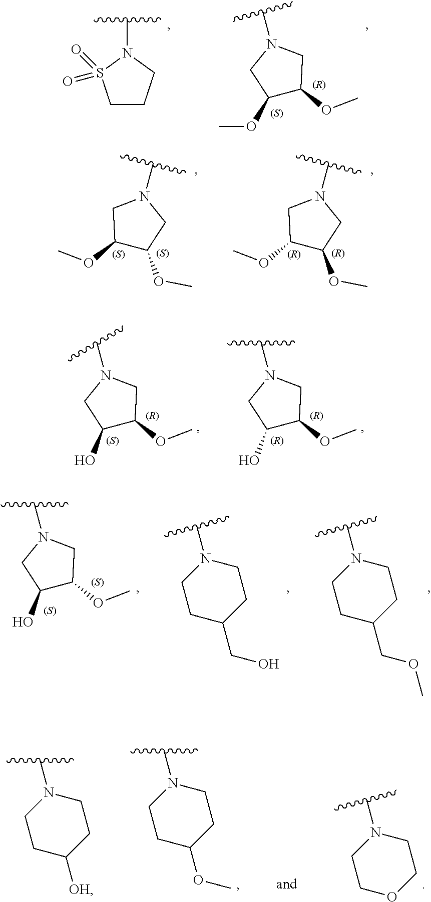

Embodiment 17. The compound of Embodiment 16, or a pharmaceutically acceptable salt thereof, wherein

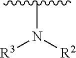

is selected from the group consisting of:

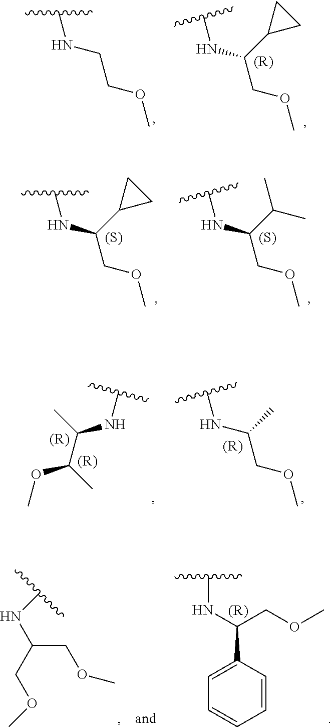

Embodiment 18. The compound of Embodiment 16, or a pharmaceutically acceptable salt thereof, wherein

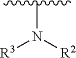

is selected from the group consisting of:

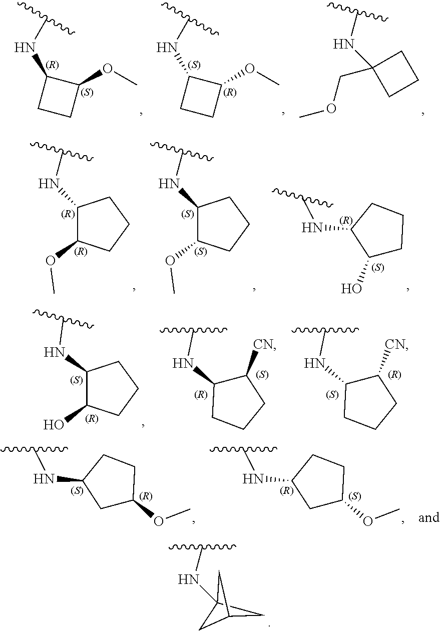

Embodiment 19. The compound of Embodiment 16, or a pharmaceutically acceptable salt thereof, wherein

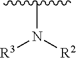

is selected from the group consisting of:

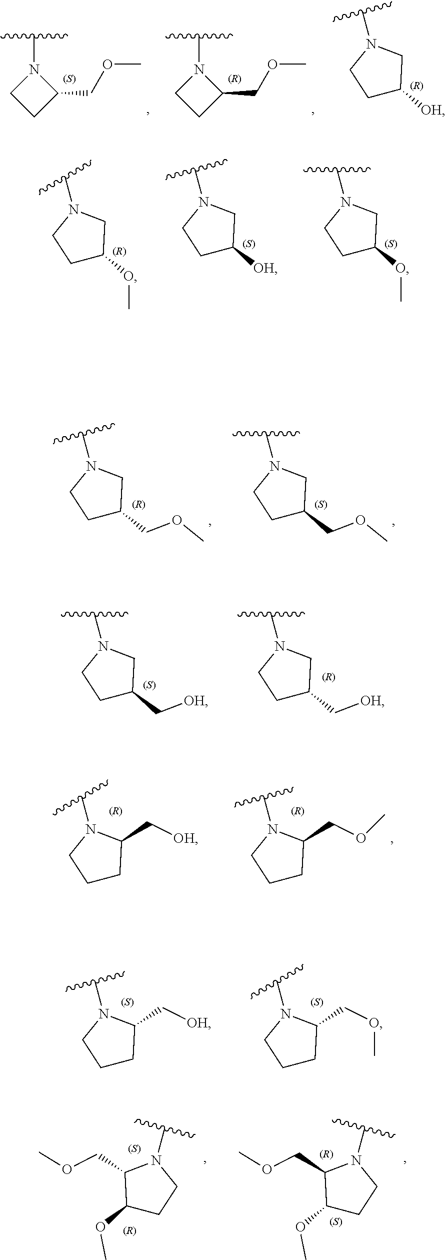

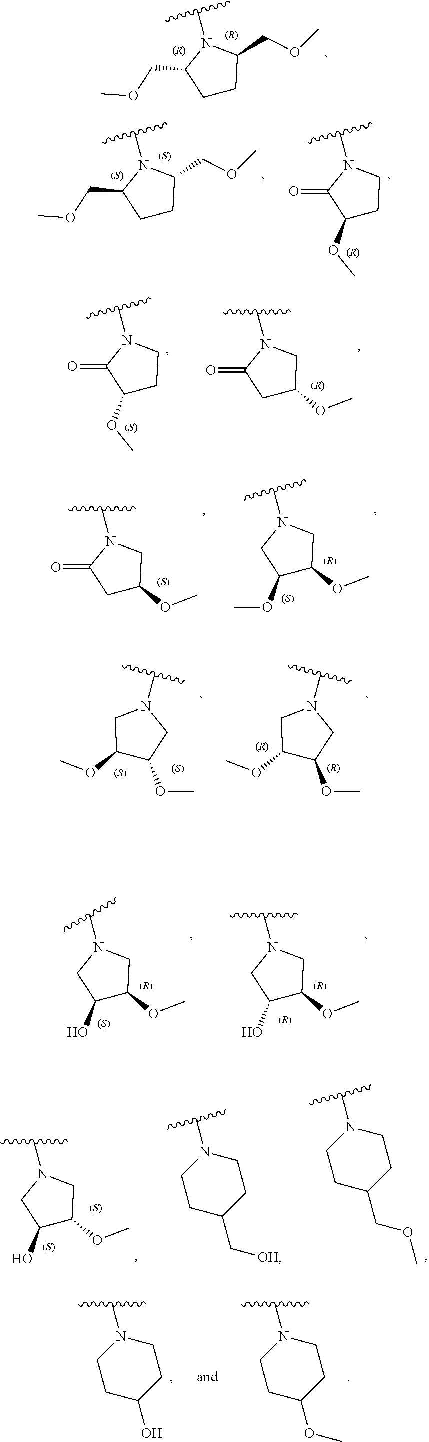

Embodiment 20. The compound of any one of Embodiments 1-19, or pharmaceutically acceptable salt thereof, wherein Ring A is:

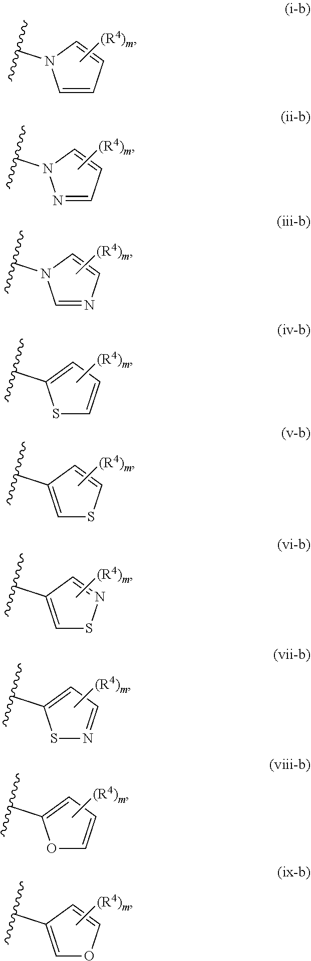

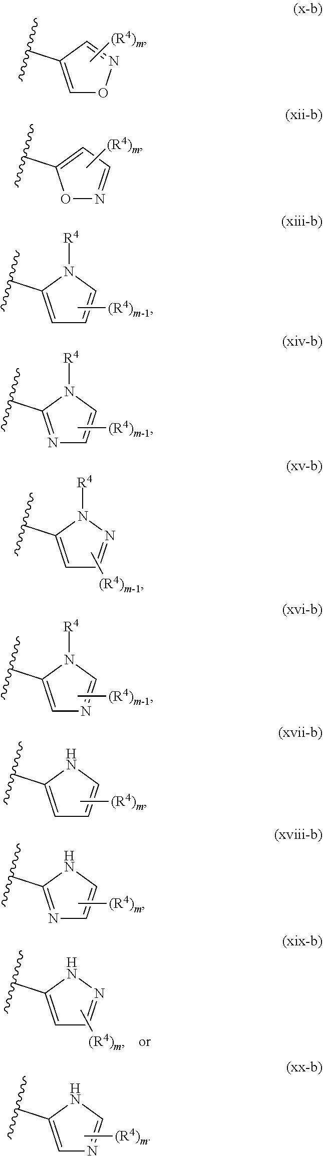

Embodiment 21. The compound of Embodiment 20, or a pharmaceutically acceptable salt thereof, wherein Ring A is:

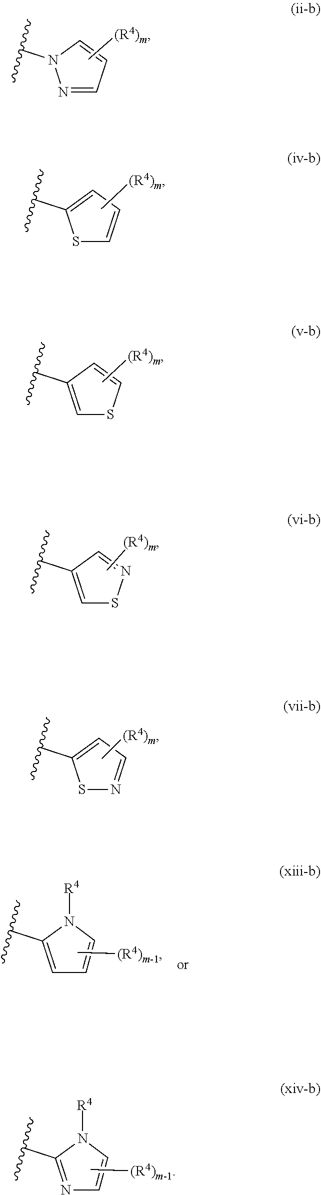

Embodiment 22. The compound of any one of Embodiments 1-21, or a pharmaceutically acceptable salt thereof, wherein each R⁴ is independently halogen, —CN, -L₂-OR^{4A}, -L₂-N(R^{4B})₂, C₁-C₆ alkyl, or C₁-C₆ haloalkyl.

Embodiment 23. The compound of Embodiment 22, or a pharmaceutically acceptable salt thereof, wherein each R⁴ is independently —CH₃, —CH₂CH₃, —CHF₂, —CF₃, —Cl, —CN, —NH₂, or —CH₂OH.

Embodiment 24. The compound of any one of Embodiments 1-23, or a pharmaceutically acceptable salt thereof, wherein L₁ is a bond or C₁-C₃ alkylene.

Embodiment 25. The compound of any one of Embodiments 1-24, or a pharmaceutically acceptable salt thereof, wherein L₂ is a bond or C₁-C₃ alkylene.

Embodiment 26. The compound of any one of Embodiments 1-25, or a pharmaceutically acceptable salt thereof, wherein m is 1.

Embodiment 27. The compound of any one of Embodiments 1-26, or a pharmaceutically acceptable salt thereof, wherein m is 2.

Embodiment 28. The compound of Embodiment 20, or a pharmaceutically acceptable salt thereof, wherein Ring A is:

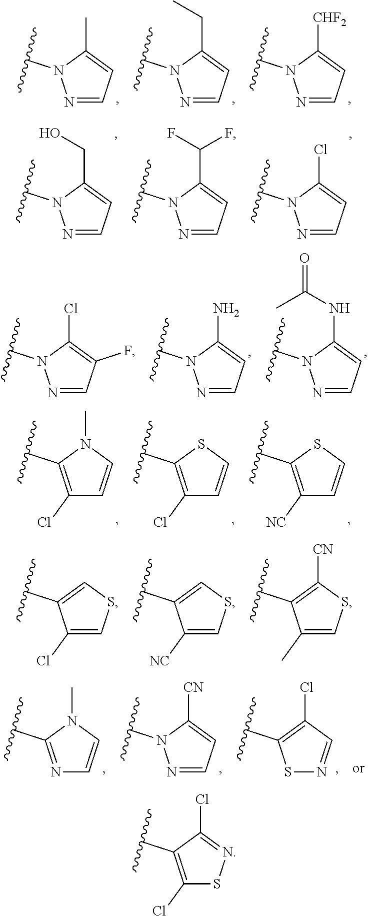

Embodiment 29. The compound of Embodiment 28, or a pharmaceutically acceptable salt thereof, wherein Ring A is:

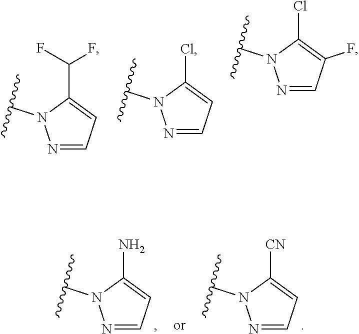

Embodiment 30. The compound of Embodiment 1 or 2, wherein the compound is of Formula (I-a), (I-b), (I-c), (I-d), (I-e), (I-f), (I-g), (I-a-1), (I-a-2), (I-a-3), (I-f-1), (I-f-2), (I-f-3), (I-BC-a), or (I-BC-b):

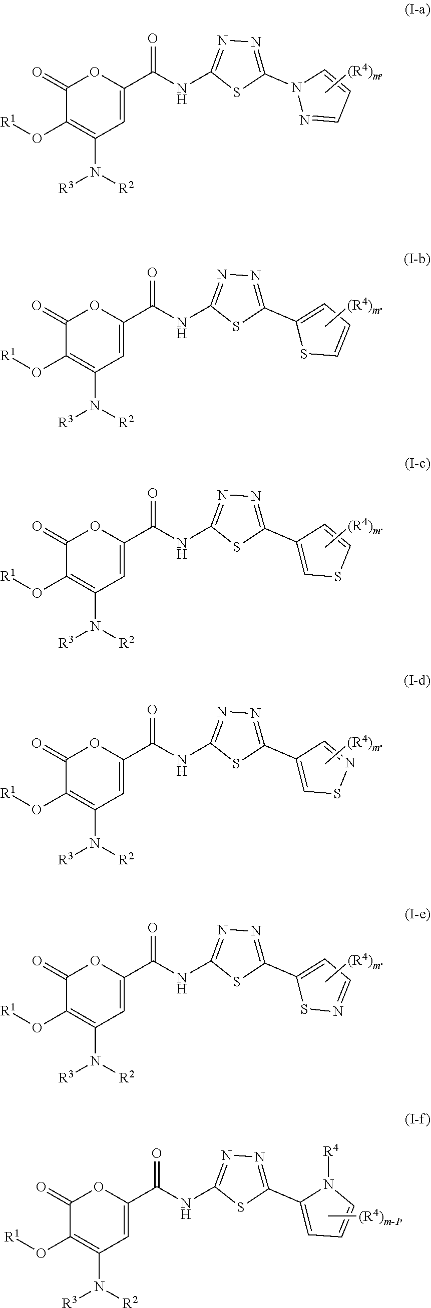

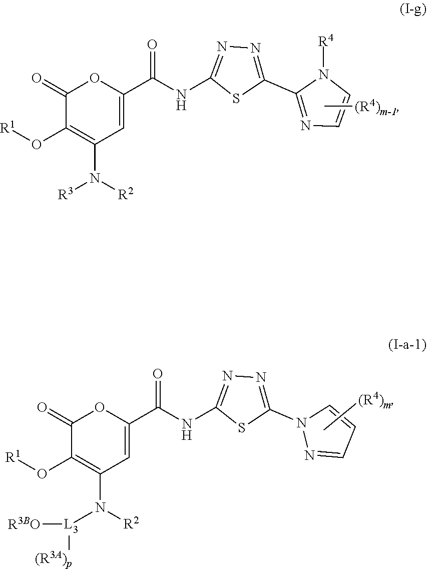

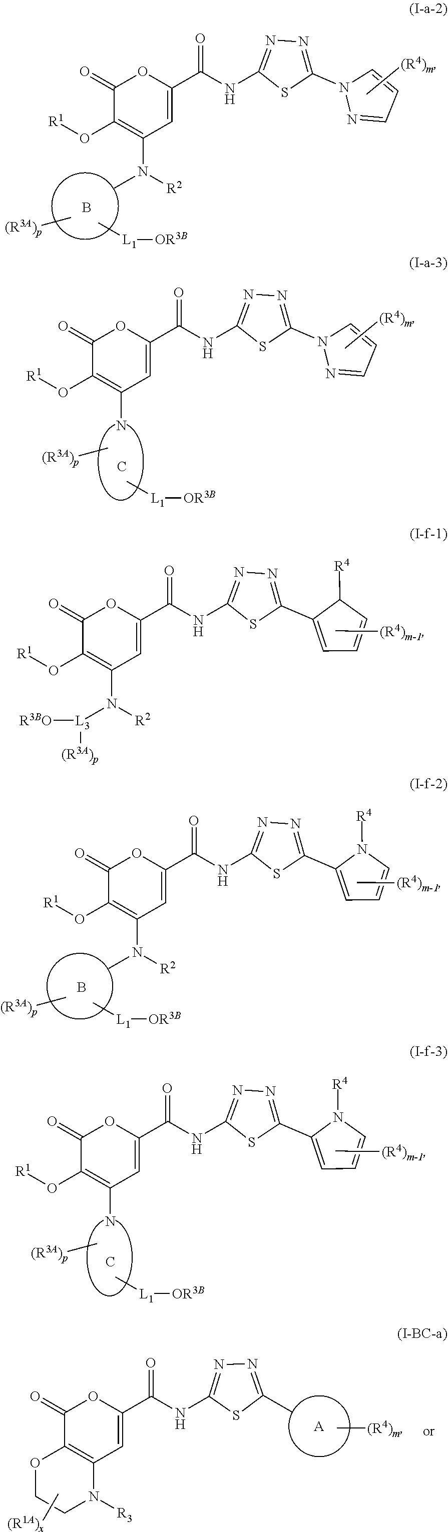

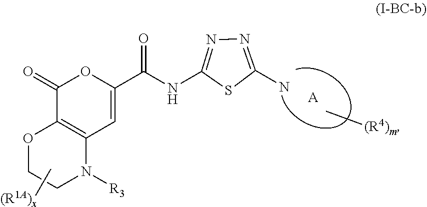

or a pharmaceutically acceptable salt thereof, wherein x is 0, 1, 2, 3, or 4.

Embodiment 31. The compound of Embodiment 2, or pharmaceutically acceptable salt thereof, wherein:
R¹ is C₁-C₆ alkyl, substituted with 0, 1, 2, 3, or 4 R^{1A}; each R^{1A} is independently-OR^{1B}; and
   each R^{1B} is independently hydrogen or C₁-C₃alkyl;
R² is hydrogen or C₁-C₆ alkyl substituted with 0, 1, 2, 3, or 4 R^{2A}, and each R^{2A} is independently —OR^{2B}, wherein each R^{2B} is independently hydrogen or C₁-C₃ alkyl;
R³ is C₁-C₁₀ alkyl, C₃-C₁₀ carbocyclyl, or 4- to 10-membered heterocyclyl, wherein the alkyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, halogen, ═O, -L₁-CN, -L₁-SO₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, carbocyclyl, heterocyclyl, aryl, and heteroaryl are independently substituted with 0, 1, 2, 3, or 4 R^{3D};
each R^{3C} is independently C₁-C₃ alkyl;
each R^{3D} is independently halogen or —OR^{3E};
R^{3E} is C₁-C₃ alkyl;
each R⁴ is independently halogen, —CN, -L₂-OR^{4A}, -L₂-N(R⁴)₂, C₁-C₆ alkyl, or C₁-C₆ haloalkyl;
each L₁ and L₂ is independently a bond or C₁-C₃ alkylene; and
m is 1 or 2.

Embodiment 32. The compound of Embodiment 1 or 2, wherein the compound is a compound of Table 1 or Table 2, or a pharmaceutically acceptable salt thereof.

Embodiment 33. The compound of Embodiment 32, wherein the compound is Compound 3, Compound 4, Compound 10, Compound 21, Compound 22, Compound 67a, Compound 67b, Compound 73, Compound 74, Compound 77, Compound 83, Compound 107a, Compound 107b, Compound 108a, Compound 108b, Compound 114, Compound 121, Compound 127, Compound 161, Compound 182, Compound 196, Compound 197, Compound 213, or a pharmaceutically acceptable salt thereof.

Embodiment 34. A method of preparing a compound of Formula (I):

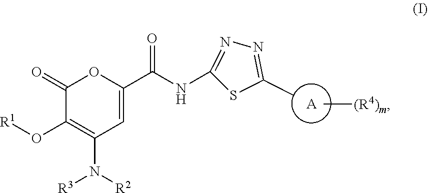

or a salt thereof, wherein Ring A, R¹, R², R³, R⁴, and m are defined in Embodiments 1 or 2, the method comprising peptide coupling of a compound of Formula (H-1), or salt thereof, with a compound of Formula (N), or salt thereof:

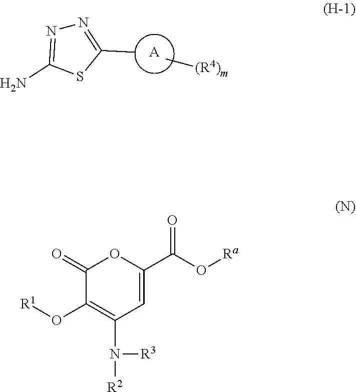

wherein R^{a} is hydrogen, C₁₋₆ alkyl or C₁₋₆ haloalkyl, to provide a compound of Formula (I), or salt thereof.

Embodiment 35. The method of Embodiment 34, wherein the compound of Formula (H-1), or salt thereof, is of Formula (H-2):

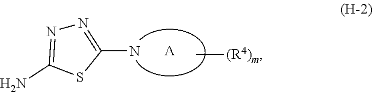

or salt thereof,
and wherein the method provides a compound of Formula (I″″):

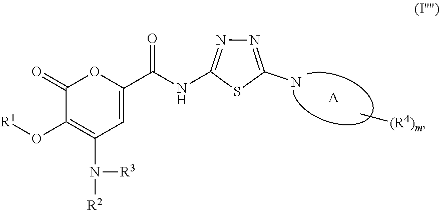

or salt thereof.

Embodiment 36. The method of Embodiment 34 or 35, further comprising cross-coupling a compound of Formula (K), or salt thereof, with a compound of Formula (D), or salt thereof,

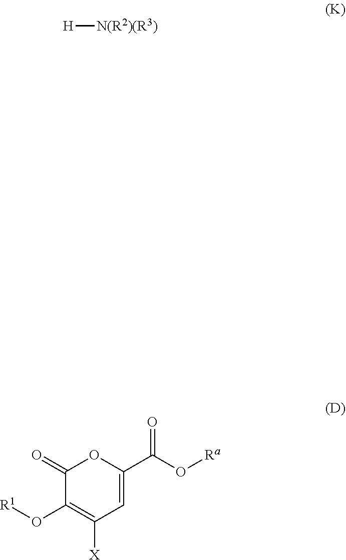

wherein R^{a} is C₁₋₆ alkyl or C₁₋₆ haloalkyl and X is Cl, Br, or I, to provide a compound of Formula (N), or salt thereof.

Embodiment 37. A method of preparing a compound of Formula (I):

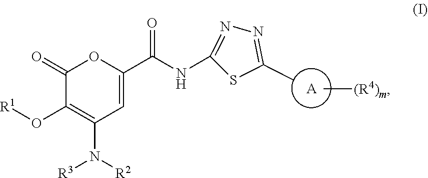

or a salt thereof, wherein Ring A, R¹, R², R³, R⁴, and m are defined in Embodiments 1 or 2, the method comprising cross-coupling of the amine of Formula (K), or salt thereof, with a compound of Formula (J-1), or salt thereof:

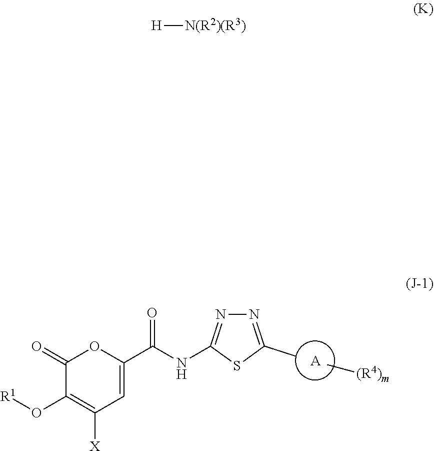

wherein X is Cl, Br, or I, to provide a compound of Formula (I), or salt thereof.

Embodiment 38. The method of Embodiment 37, wherein the compound of Formula (J-1), or salt thereof, is of Formula (J-2):

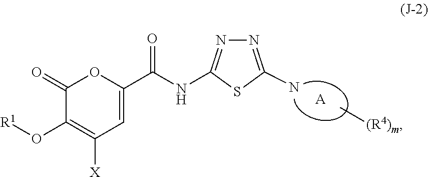

or salt thereof.
and wherein the method provides a compound of Formula (I″″):

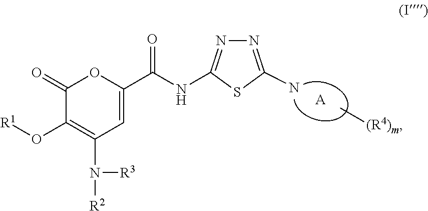

or salt thereof.

Embodiment 39. The method of Embodiment 37, further comprising peptide coupling of a compound of Formula (H-1), or salt thereof, with a compound of Formula (D), or salt thereof:

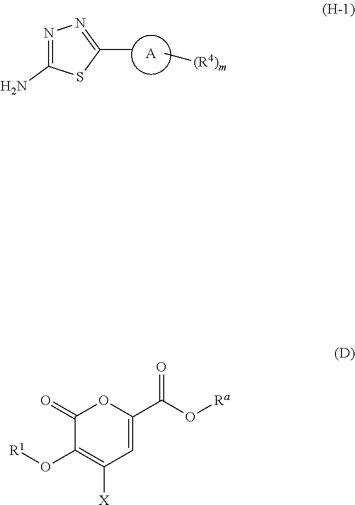

to provide a compound of Formula (J-1), or salt thereof.

Embodiment 40. The method of Embodiment 38, further comprising peptide coupling of a compound of Formula (H-2), or salt thereof, with a compound of Formula (D), or salt thereof:

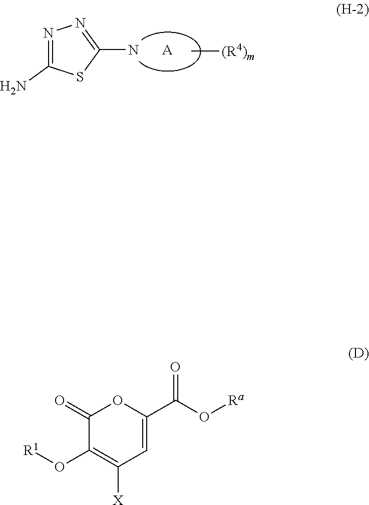

to provide a compound of Formula (J-2), or salt thereof.

Embodiment 41. The method of Embodiment 33 or 39, further comprising reacting a hydrazine carbothioamide of Formula (G), or salt thereof, with a carboxylic acid containing compound of Formula (F-1), or salt thereof, or nitrile containing compound of Formula (F-2), or salt thereof:

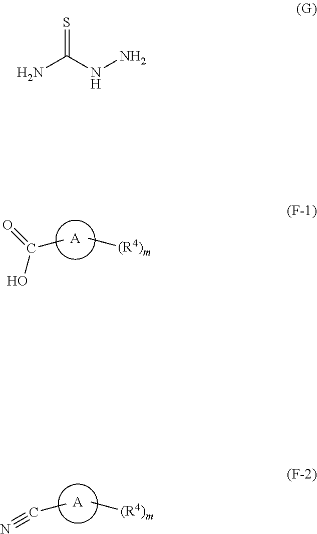

to provide a compound of Formula (H-1), or salt thereof.

Embodiment 42. The method of Embodiment 35 or 40, further comprising coupling a compound Formula (M), or salt thereof, wherein Y is Cl, Br, or I, with an amine of Formula (L), or salt thereof:

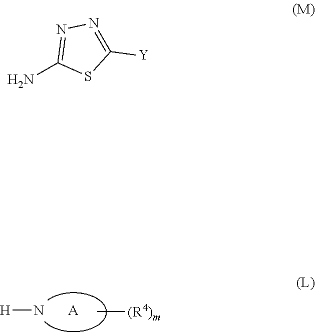

to provide a compound of Formula (H-2), or salt thereof.

Embodiment 43. The method of any one of Embodiments 36, 39, and 40, further comprising:
(a) protecting a compound of Formula (A), or salt thereof, to provide an alkyl ester of Formula (B):

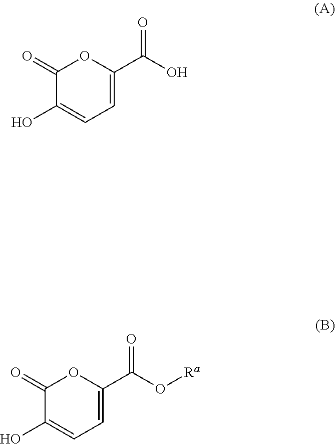

or salt thereof, wherein R^{a} is C₁₋₆ alkyl or C₁₋₆ haloalkyl;
(b) halogenating the compound of Formula (B), or salt thereof, to provide a compound of Formula (C):

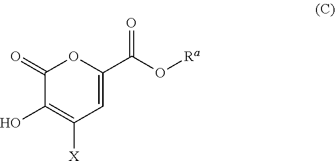

or salt thereof, wherein X is Cl, Br, or I;
(c) protecting the hydroxyl group of the compound of Formula (C), or salt thereof, to provide a compound of Formula (D):

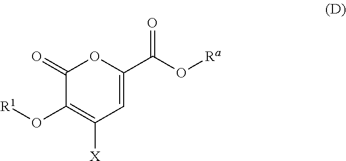

or salt thereof, wherein R¹ is as defined in Embodiment 1; and
(d) optionally, deprotecting the compound of Formula (D), or salt thereof, to provide a carboxylic acid of Formula (D), wherein R^{a} is hydrogen.

Embodiment 44. A method of preparing a compound of Formula (I-BC-a):

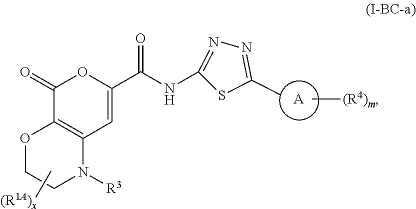

or salt thereof;
wherein Ring A, R¹, R³, R⁴, and m are defined in Embodiment 1, the method comprising cyclizing a compound of Formula (P-1):

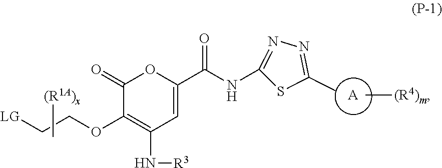

or salt thereof.
wherein LG is a leaving group.

Embodiment 45. The method of Embodiment 44, wherein the compound of Formula (I-BC-a), or salt thereof, is of Formula (I-BC-b):

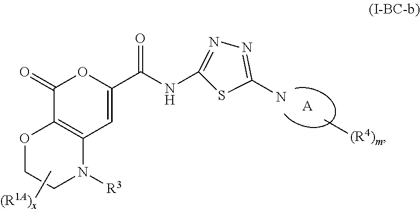

or salt thereof;
and the compound of Formula (P-1) is of Formula (P-2):

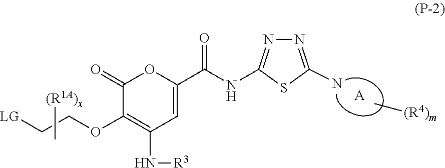

or salt thereof.

Embodiment 46. The method of Embodiment 44 or 45, wherein the compound of Formula (P-1), or salt thereof, or compound of Formula (P-2), or salt thereof, is prepared by conversion of the terminal —OH of Formula (I-X-1) or of Formula (I-X-2):

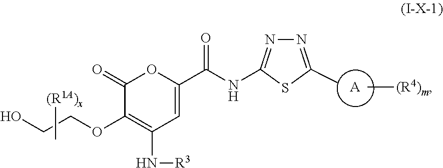

or salt thereof,

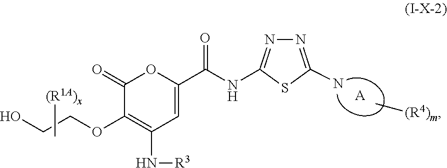

or salt thereof, to a leaving group.

Embodiment 47. A pharmaceutical composition comprising the compound of any one of Embodiments 1-33, or a pharmaceutically acceptable salt thereof, and a pharmaceutically acceptable carrier.

Embodiment 48. A method of treating a disease or disorder in a subject in need thereof, comprising administering to the subject a compound of any one of Embodiments 1-33, or a pharmaceutically acceptable salt thereof, or a pharmaceutical composition of Embodiment 47.

Embodiment 49. The method of Embodiment 48, wherein the disease or disorder is inflammation, an autoimmune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an cGAS-related disease in a subject that has been determined to carry a germline or somatic non-silent mutation in cGAS.

Embodiment 50. A method of modulating cGAS activity, comprising contacting a cell with a compound of any one of Embodiments 1-33, or a pharmaceutically acceptable salt thereof, or a pharmaceutical composition of Embodiment 47.

### EXEMPLIFICATION

In order that this disclosure may be more fully understood, the following Examples are set forth. It should be understood that these examples are for illustrative purposes only and are not to be construed as limiting this disclosure in any manner.

### Analytical Methods

Nuclear magnetic resonance (NMR) spectra were recorded at 400 MHz as stated and at 300.3 K unless otherwise stated; the chemical shifts (δ) are reported in parts per million (ppm). Spectra were recorded using a Bruker Avance 400 instrument with 8, 16 or 32 scans. Typical NMR solvents include deuterated dimethylsulfoxide (DMSO-d₆) and deuterated methanol (CD₃OD).

Liquid Chromatography-Mass Spectrometry (LCMS) chromatograms and spectra were recorded using a Shimadzu LCMS-2020. Injection volumes were 0.7-8.0 μl and the flow rates were typically 0.8 or 1.2 mL/min. Detection methods were diode array (DAD) or evaporative light scattering (ELSD) as well as positive ion electrospray ionization. MS range was 100-1000 Da. Mobile phases of water and/or acetonitrile (MeCN) may contain a modifier (typically 0.01-0.04%) such as trifluoroacetic acid (TFA), formic acid (FA), or ammonium carbonate (NH₄HCO₃). ESI or ES=electrospray ionization; m/z=mass/charge; RT=retention time (minutes).

Purification/Separation Methods. The Synthetic methods describe purification and/or separation chromatographic methods which have been employed in the purification and/or isolation of the exemplified compounds. Rf=retention factor; RT=retention time (minutes); Prep-HPLC=Preparative High-performance liquid chromatography.

The Asterix (*) next to the Compound Number (#) signifies that arbitrary stereochemistry has been assigned. Future tense (“may be” prepared/synthesized) language signify examples to be conducted.

### Synthetic Methods

### Example 1: 4-((1,3-dimethoxypropan-2-yl)amino)-3-methoxy-N-(5-(5-methyl-1H-pyrazol-1-yl)-1,3,4-thiadiazol-2-yl)-2-oxo-2H-pyran-6-carboxamide (Compound 10)

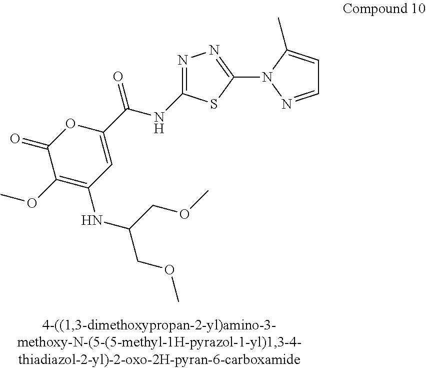 [SMILES: COCC(COC)Nc1cc(C(=O)Nc2nnc(-n3nccc3C)s2)oc(=O)c1OC]

### Example 1—Part A: Preparation of 4-bromo-3-methoxy-2-oxo-2H-pyran-6-carboxylic acid

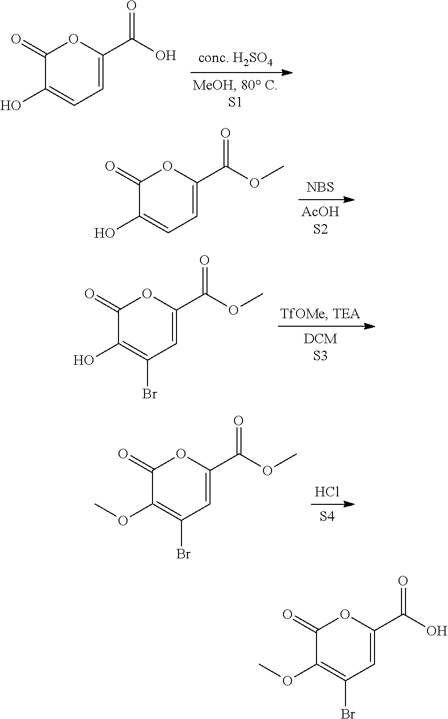

Step 1: Into a solution of 5-hydroxy-6-oxopyran-2-carboxylic acid (180 g, 1153 mmol, 1 equiv) in methanol (MeOH) (2000 mL) was added H₂SO₄ (10 mL, 56 mmol) at room temperature. Then the resulting mixture was stirred overnight at 80° C. The resulting mixture was concentrated under reduced pressure. The residue was then dissolved in ethyl acetate (EtOAc) (1000 mL) and the organic phase was washed water (3×300 mL), and dried over anhydrous Na₂SO₄. After filtration, the filtrate was concentrated under reduced pressure to provide methyl 5-hydroxy-6-oxopyran-2-carboxylate (85 g, 43% yield). LCMS (ES, m/z)=171 [M+1]+.

Step 2: To a stirred solution of methyl 5-hydroxy-6-oxopyran-2-carboxylate (1.0 g, 5.9 mmol, 1.0 equiv) in acetic acid (AcOH) (25 mL, 323 mmol) was added N-bromosuccinimide (NBS) (1.25 g, 7.02 mmol, 1.19 equiv) at room temperature. The resulting mixture was stirred for 2 h at 80° C. then diluted with water (70 mL). The resulting mixture was extracted with ethyl acetate (EtOAc) (3×70 mL). The combined organic layers were washed with brine (2×10 mL), and dried over anhydrous Na₂SO₄. After filtration, the filtrate was concentrated under reduced pressure. The resulting residue was purified by silica gel column chromatography, eluted with petroleum ether/ethyl acetate (PE/EtOAc) (3:7) to afford methyl 4-bromo-5-hydroxy-6-oxopyran-2-carboxylate (800 mg, 55% yield). LCMS (ES, m/z)=247 [M−1]−.

Step 3: To a stirred solution of methyl 4-bromo-5-hydroxy-6-oxopyran-2-carboxy late (4.0 g, 16 mmol, 1.0 equiv) in dichloromethane (DCM) (50 mL) was added diisopropylethylamine (DIEA) (11.0 g, 85.1 mmol, 5.30 equiv) and methyl trifluoromethanesulfonate (TfOMe) (13.0 g, 79.2 mmol, 4.93 equiv) dropwise at room temperature. The resulting mixture was stirred for 4 h at room temperature. The mixture was then diluted with water (200 mL) and extracted with DCM (3×200 mL). The combined organic layers were washed with brine (2×30 mL), and dried over anhydrous Na₂SO₄. After filtration, the filtrate was concentrated under reduced pressure. The resulting residue was purified by silica gel column chromatography, eluted with petroleum ether/ethyl acetate (PE/EtOAc) (3:2) to afford methyl 4-bromo-5-methoxy-6-oxopyran-2-carboxylate (3.0 g, 71% yield). LCMS (ES, m/z)=263 [M+1]+.

Step 4: To methyl 4-bromo-5-methoxy-6-oxopyran-2-carboxylate (10.0 g, 38.02 mmol, 1.00 equiv) was added HCl (6M) (200 mL, 65.8 mmol). The mixture was stirred for 4 h at 80° C. then concentrated under reduced pressure to provide 4-bromo-5-methoxy-6-oxopyran-2-carboxylic acid (9.5 g) which was used directly without further purification. LCMS (ES, m/z)=249 [M+1]+.

### Example 1—Part B: Preparation of 5-(5-methyl-1H-pyrazol-)-yl)-1,3,4-thiadiazol-2-amine

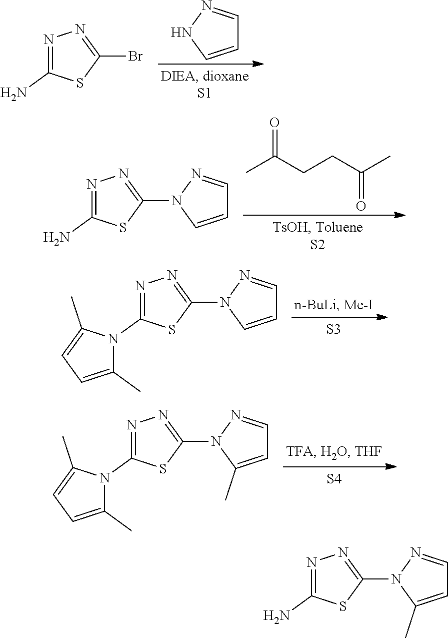

Step 1: A mixture of 5-bromo-1,3,4-thiadiazol-2-amine (200 g, 1110 mmol, 1.0 equiv), diisopropylethylamine (DIEA) (431 g, 3333 mmol, 3.0 equiv) and pyrazole (90.76 g, 1333 mmol, 1.2 equiv) in 1,4-dioxane was stirred for 3 h at 80° C. The resulting mixture was concentrated under vacuum and the residue was dissolved in tetrahydrofuran (THF). The mixture was filtered, and the filter cake was washed with tetrahydrofuran (THF). The filtrate was concentrated under reduced pressure to afford 5-(pyrazol-1-yl)-1,3,4-thiadiazol-2-amine (100 g, 54% yield), which was used directly in the next step without further purification.

Step 2: A mixture of 5-(pyrazol-1-yl)-1,3,4-thiadiazol-2-amine (100 g, 598 mmol, 1.0 equiv), tosic acid (TsOH) (20.60 g, 119.6 mmol, 0.2 equiv) and 2,5-hexanedione (102 g, 897 mmol, 1.5 equiv) in toluene was stirred for 2 h at 110° C. The resulting mixture was concentrated under vacuum and the resulting residue was purified by silica gel column chromatography eluting with petroleum ether/ethyl acetate (PE/EtOAc) (9:1) to afford 2-(2,5-dimethylpyrrol-1-yl)-5-(pyrazol-1-yl)-1,3,4-thiadiazole (90.3 g, 61% yield). LCMS (ES, m/z)=246.1 [M+1]+.

Step 3: A solution of 2-(2,5-dimethylpyrrol-1-yl)-5-(pyrazol-1-yl)-1,3,4-thiadiazole (50.0 g, 204 mmol, 1.0 equiv) in THF was treated with n-butyl lithium (n-BuLi) (97.8 mL, 245 mmol, 1.2 equiv) for 1 h at −78° C. under N₂ (nitrogen gas) followed by the addition of methyl iodide (CH₃I) (34.7 g, 245 mmol, 1.2 equiv) dropwise at −78° C. The resulting mixture was stirred for 2 h at room temperature under N₂. The reaction was quenched with sat. NH₄Cl (aq.) at 0° C. The resulting mixture was extracted with EtOAc. The combined organic layers were washed with brine and dried over anhydrous Na₂SO₄. After filtration, the filtrate was concentrated under reduced pressure. The residue was purified by silica gel column chromatography eluting with petroleum ether/ethyl acetate (PE/EtOAc) (9:1) to afford 2-(2,5-dimethylpyrrol-1-yl)-5-(5-methylpyrazol-1-yl)-1,3,4-thiadiazole (40 g, 76% yield). LCMS (ES, m/z)=260.0 [M+1]+.

Step 4: To a solution of 2-(2,5-dimethylpyrrol-1-yl)-5-(5-methylpyrazol-1-yl)-1,3,4-thiadiazole (7.0 g, 27 mmol, 1.0 equiv) in tetrahydrofuran (THF) (14 mL) and H₂O (28 mL) at room temperature was added trifluoroacetic acid (TFA) (28 mL). The resulting mixture was stirred for 2 h at 50° C. then concentrated under reduced pressure. The residue was purified by C18 reverse phase flash chromatography (acetonitrile (MeCN) in water, 10% to 20% gradient in 10 min; Wave Length: 254 nm) to afford 5-(5-methylpyrazol-1-yl)-1,3,4-thiadiazol-2-amine (also referred to as 5-(5-methyl-1H-pyrazol-1-yl)-1,3,4-thiadiazol-2-amine) (3.0 g, 58% yield). LCMS (ES, m/z)=181.95 [M+1]+.

### Example 1—Part C: Preparation of 4-((1,3-dimethoxypropan-2-yl)amino)-3-methoxy-N-(5-(5-methyl-1H-pyrazol-1-yl)-1,3,4-thiadiazol-2-yl)-2-oxo-2H-pyran-6-carboxamide

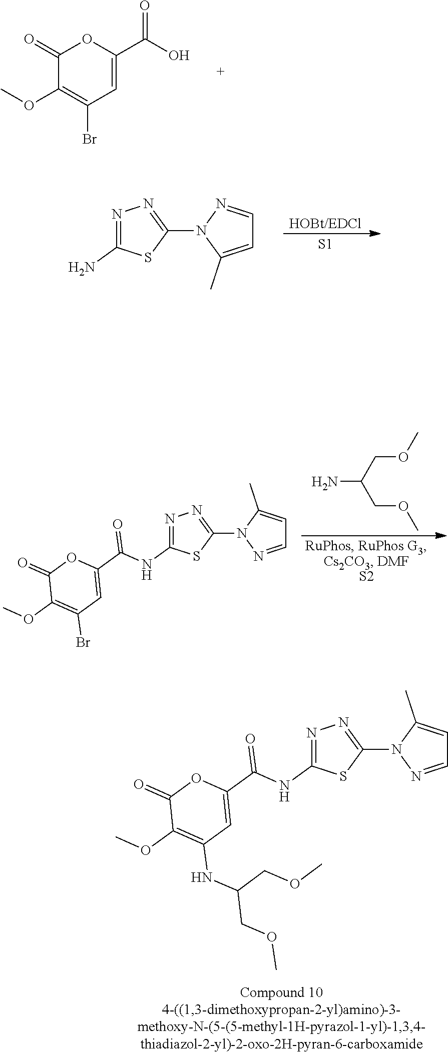

Step 1: To a stirred solution of 4-bromo-5-methoxy-6-oxopyran-2-carboxylic acid (product of Example 1, Part A, Step 4) (12.0 g, 48.2 mmol, 1.00 equiv) in N,N-dimethylformamide (DMF) (150 mL) was added hydroxybenzotriazole (HOBt) (13.02 g, 96.38 mmol, 2.00 equiv), 1-ethyl-3-(3-dimethylaminopropyl)carbodiimide (EDCI) (27.81 g, 145.05 mmol, 3.01 equiv) and 5-(5-methylpyrazol-1-yl)-1,3,4-thiadiazol-2-amine (product of Example 1, Part B, Step 4) (9.00 g, 49.7 mmol, 1.03 equiv) at room temperature. The resulting mixture was stirred for 1 h at room temperature then quenched by the addition of water (70 mL). The precipitated solids were collected by filtration and washed with acetonitrile (5×3 mL) to provide 4-bromo-5-methoxy-N-[5-(5-methylpyrazol-1-yl)-1,3,4-thiadiazol-2-yl]-6-oxopyran-2-carboxamide (11.0 g, 55% yield). LCMS (ES, m/z)=412 [M+1]+.

Step 2: To a stirred solution of 4-bromo-5-methoxy-N-[5-(5-methylpyrazol-1-yl)-1,3,4-thiadiazol-2-yl]-6-oxopyran-2-carboxamide (also referred to herein as 4-bromo-3-methoxy-N-(5-(5-methyl-1H-pyrazol-1-yl)-1,3,4-thiadiazol-2-yl)-2-oxo-2H-pyran-6-carboxamide) (100 mg, 0.243 mmol, 1.0) equiv) (“halo-pyrone reagent”) in N,N-dimethylformamide (DMF) (3.5 mL) was added 1,3-dimethoxypropan-2-amine (“amine reagent”) (60 mg, 0.50 mmol, 2.1 equiv), 2-dicyclohexylphosphino-2′,6′-diisopropoxybiphenyl (RuPhos) (40 mg, 0.086 mmol, 0.35 equiv), (2-dicyclohexylphosphino-2′,6′-diisopropoxy-1,1′-biphenyl)[2-(2′-amino-1,1′-biphenyl)]palladium(II) methanesulfonate (RuPhos Palladacycle Gen3) (40 mg, 0.048 mmol, 0.20 equiv) and Cs₂CO₃ (240 mg, 0.737 mmol, 3.04 equiv) at room temperature. The resulting mixture was stirred for 3 h at 100° C. under N₂ (nitrogen gas). The resulting mixture was filtered, and the filter cake was washed with acetonitrile (1×3 mL) and the filtrate was concentrated under reduced pressure. The resulting residue was purified by C18 reverse phase flash chromatography (acetonitrile (MeCN) in water, 10% to 50% gradient in 10 min; Wave Length: 254 nm) followed by additional purification by Chiral-Prep-HPLC (conditions: Xselect CSH C18 OBD Column 30*150 mm 5 um; Mobile phase, acetonitrile (MeCN) and water (29% Water+0.05% trifluoroacetic acid (TFA)) up to 39% in 10 min, hold 39% in 2 min); Wave Length: 254 nm) to provide 4-((1,3-dimethoxypropan-2-yl)amino)-3-methoxy-N-(5-(5-methyl-1H-pyrazol-1-yl)-1,3,4-thiadiazol-2-yl)-2-oxo-2H-pyran-6-carboxamide (Compound 10) (25.4 mg, 23.2% yield). LCMS (ES, m/z)=451.20 [M+1]+. ¹H NMR (400 MHz, DMSO-d6) δ 7.78 (d, J=1.6 Hz, 1H), 7.44 (s, 1H), 6.44 (d, J=1.6 Hz, 1H), 4.14-4.09 (m, 1H), 3.69 (s, 3H), 3.50-3.43 (m, 4H), 3.38 (s, 6H), 2.68 (s, 3H).

### Example 2: 3-methoxy-4-(((1R, 2R)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 3*) and 3-methoxy-4-(((1S, 2S)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 4*)

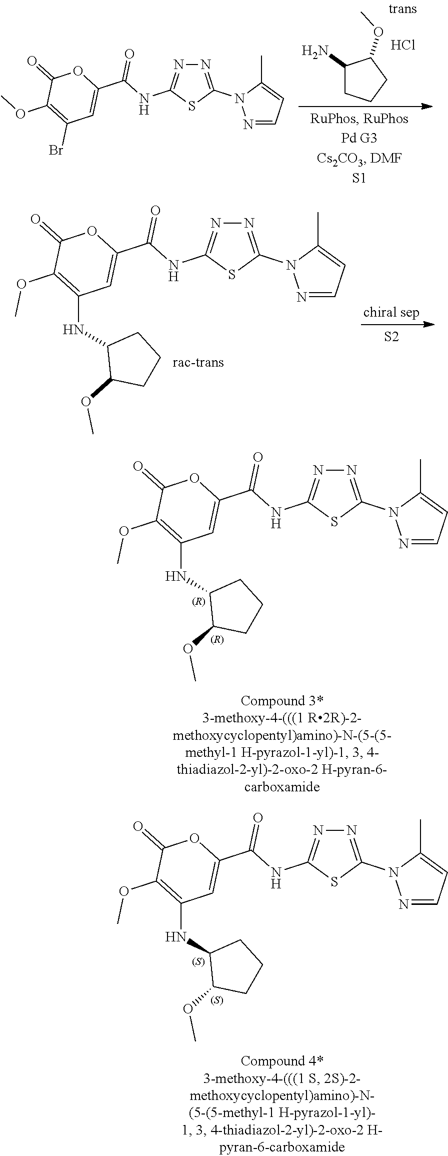

Racemic trans-3-methoxy-4-((-2-methoxycyclopentyl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using trans-2-methoxycyclopentan-1-amine hydrochloride as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1, as the “halo-pyrone reagent”). Separation of constituent enantiomers of racemic trans-3-methoxy-4-((-2-methoxycyclopentyl)amino)-N-(5-(5-methyl-1H-pyrazol-1-yl)-1,3,4-thiadiazol-2-yl)-2-oxo-2H-pyran-6-carboxamide by prep-Chiral-HPLC (conditions: Column: CHIRAL ART Amylose-SA, 2*25 cm, 5 μm; Mobile Phase A: Hexanes (0.1% trifluoroacetic acid (TFA)), Mobile Phase B: methanol (MeOH):dichloromethane (DCM)=1:1; Flow rate: 20 mL/min; Gradient: 60% B to 60% B in 10 min; Wave Length: 220/254 nm; RT1 (min): 6.99; RT2 (min): 9.03; Sample Solvent: MeOH) provided two enantiomers with arbitrarily assigned stereochemistry: 3-methoxy-4-(((1R, 2R)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 3*), first eluting peak. LCMS (ES, m/z)=447.10 [M+1]+, ¹H NMR (400 MHz, DMSO-d6) δ 13.32 (br, 1H), 7.79 (d, J=1.6 Hz, 1H), 7.42 (s, 1H), 7.01 (d, J=8.4 Hz, 1H), 6.45 (d, J=1.6 Hz, 1H), 3.96-3.92 (m, 1H), 3.79-3.68 (m, 1H), 3.69 (s, 3H), 3.25 (s, 3H), 2.68 (s, 3H), 2.10-1.90 (m, 2H), 1.73-1.51 (m, 4H); and 3-methoxy-4-(((1S, 2S)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 4*), second eluting peak. LCMS (ES, m/z)=447.10 [M+1]+. ¹H NMR (400 MHz, DMSO-d6) δ 13.32 (br, 1H), 7.79 (d, J=1.6 Hz, 1H), 7.42 (s, 1H), 7.01 (d, J=8.4 Hz, 1H), 6.45 (d, J=1.2 Hz, 1H), 3.93 (d, J=8.0 Hz, 1H), 3.75 (q, J=5.6 Hz, 1H), 3.69 (s, 3H), 3.25 (s, 3H), 2.68 (s, 3H), 2.10-1.90 (m, 2H), 1.73-1.51 (m, 4H).

### Example 3: 4-(((cis)-2-hydroxycyclopentyl)amino)-3-methoxy-N-(5-(5-methyl-1H-pyrazol-1-yl)-1,3,4-thiadiazol-2-yl)-2-oxo-2H-pyran-6-carboxamide (Compound 23, Rac-23), 4-(((1S,2R)-2-hydroxycyclopentyl)amino)-3-methoxy-N-(5-(5-methyl-1H-pyrazol-1-yl)-1,3,4-thiadiazol-2-yl)-2-oxo-2H-pyran-6-carboxamide (Compound 23a*) and 4-(((1R,2S)-2-hydroxycyclopentyl)amino)-3-methoxy-N-(5-(5-methyl-1H-pyrazol-1-yl)-1,3,4-thiadiazol-2-yl)-2-oxo-2H-pyran-6-carboxamide (Compound 23b*)

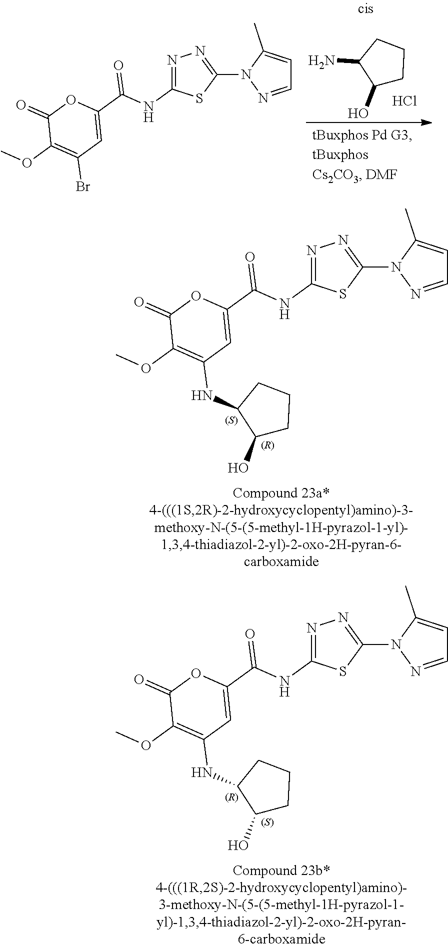

To a stirred solution of 4-bromo-5-methoxy-N-[5-(5-methylpyrazol-1-yl)-1,3,4-thiadiazol-2-yl]-6-oxopyran-2-carboxamide (100 mg, 0.243 mmol, 1 equiv) (product of Example 1, Part C, Step 1; “halo-pyrone reagent”) in N,N-dimethylformamide (DMF) (1.5 mL) was added cis-2-aminocyclopentan-1-ol hydrochloride (81 mg, 0.59 mmol, 2.45 equiv) (“amine reagent”), [(2-di-tert-butylphosphino-2′,4′,6′-triisopropyl-1,1′-biphenyl)-2-(2′-amino-1,1′-biphenyl)] palladium(II) methanesulfonate (tBuxphos Pd G3) (39 mg, 0.049 mmol, 0.20 equiv), di-tert-butyl-[2-(2,4,6-triisopropylphenyl)phenyl]phosphane (tBuxphos) (32 mg, 0.075 mmol, 0.31 equiv) and cesium carbonate (230 mg, 0.71 mmol, 2.91 equiv) at room temperature. The resulting mixture was stirred for 1 h at 100° C. under N₂ (nitrogen gas). The resulting mixture was filtered and the filter cake was washed with acetonitrile (MeCN) (3×3 mL). The filtrate was concentrated under reduced pressure and the residue was purified by reverse flash chromatography (conditions: Mobile phase: MeCN in water, 10% to 50% gradient in 10 min; Wave Length: 254 nm). Additional purification by Prep-HPLC (conditions: XBridge Prep Phenyl OBD Column, 19*150 mm, 5 μm; Mobile Phase A: water (10 mmol/L NH₄HCO₃). Mobile Phase B: MeCN; Flow rate: 60 mL/min; Gradient: 13% B to 27% B in 8 min. 27% B; Wave Length: 254 nm) provided a racemic mixture of the title compounds (Compound 23a* and Compound 23b*) (7.2 mg, 6.8% yield). LCMS (ES, m/z)=433.15 [M+1]+. ¹H NMR (400 MHz, DMSO-d₆) δ 7.75 (d, J=1.6 Hz, 1H), 7.32 (s, 1H), 6.42 (d, J=1.6 Hz, 1H), 6.15 (br, 1H), 5.14 (d, J=4.8 Hz, 1H), 4.10-4.05 (m, 1H), 3.92-3.85 (m, 1H), 3.73 (s, 3H), 2.67 (s, 3H), 2.04-1.97 (m, 11H), 1.89-1.77 (m, 2H), 1.62-1.57 (m, 311).

### Example 4: N-(5-(4-chlorothiophen-3-yl)-1,3,4-thiadiazol-2-yl)-3-methoxy-4-((2-methoxyethyl)amino)-2-oxo-2H-pyran-6-carboxamide (Compound 136)

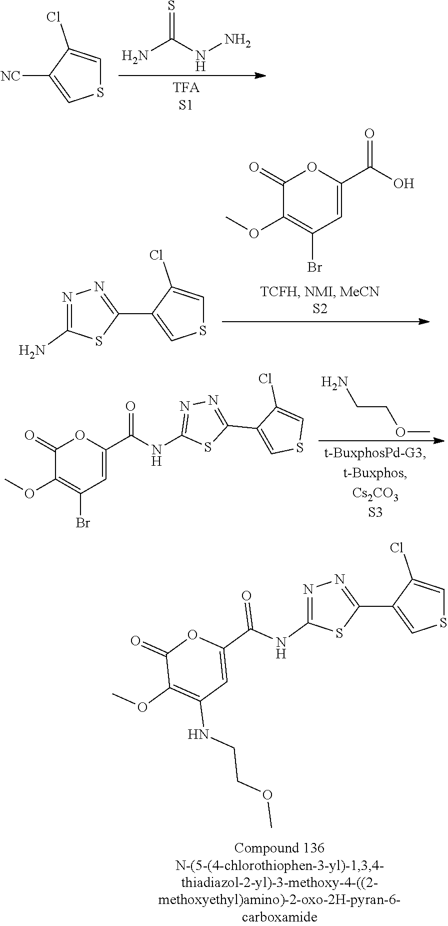

Step 1: To a stirred solution of 4-chlorothiophene-3-carbonitrile (270 mg, 1.88 mmol, 1.00 equiv) in trifluoroacetic acid (TFA) (3.00 mL) was added thiosemicarbazide (257 mg, 2.82 mmol, 1.50 equiv) at room temperature. The resulting mixture was stirred for 2 h at 80° C. The resulting mixture was concentrated under reduced pressure. The crude product was purified by C18 reverse phase flash chromatography (acetonitrile (MeCN)/H₂O=3:2) to afford 5-(4-chlorothiophen-3-yl)-1,3,4-thiadiazol-2-amine (260 mg, 64% yield). LCMS (ES, m/z)=218.0 [M+1]+.

Step 2: To a stirred solution of 5-(4-chlorothiophen-3-yl)-1,3,4-thiadiazol-2-amine (200 mg, 0.92 mmol, 1.00 equiv) and 4-bromo-5-methoxy-6-oxopyran-2-carboxylic acid (product of Example 1, Part A, Step 4) (343 mg, 1.38 mmol, 1.50 equiv) in acetonitrile (MeCN) (2.00 mL) was added N-methylimidazole (NMI) (377 mg, 4.60 mmol, 5.00 equiv) and chloro-N,N,N′,N′-tetramethylformamidinium hexafluorophosphate (TCFH) (335 mg, 1.20 mmol, 1.3 equiv) at room temperature. The resulting mixture was stirred for 1 h at room temperature then concentrated under reduced pressure. The residue was purified by C18 reverse phase chromatography (MeCN/Water=3:1) to afford 4-bromo-N-[5-(4-chlorothiophen-3-yl)-1,3,4-thiadiazol-2-yl]-5-methoxy-6-oxopyran-2-carboxamide (80 mg, 18% yield). LCMS (ES, m/z)=448.0 [M+1]+.

Step 3: N-(5-(4-Chlorothiophen-3-yl)-1,3,4-thiadiazol-2-yl)-3-methoxy-4-((2-methoxyethyl)amino)-2-oxo-2H-pyran-6-carboxamide (Compound 136) was prepared from 4-bromo-N-[5-(4-chlorothiophen-3-yl)-1,3,4-thiadiazol-2-yl]-5-methoxy-6-oxopyran-2-carboxamide as the “halo-pyrone reagent” and 2-methoxyethan-1-amine as the “amine reagent” according to the procedure outlined for the preparation of Compound 23 in Example 3. LCMS (ES, m/z)=443.0 [M+1]+. ¹H NMR (400 MHz, Methanol-d₆) δ 8.10 (s, 1H), 7.57 (s, 1H), 7.35 (s, 1H), 3.79 (s, 3H), 3.66-3.57 (m, 4H), 3.40 (s, 3H).

### Example 5: N-(5-(3-chlorothiophen-2-yl)-1,3,4-thiadiazol-2-yl)-4-(((cis)-2-hydroxycyclopentyl)amino)-3-methoxy-2-oxo-2H-pyran-6-carboxamide (Compound 7, Rac-7), N-(5-(3-chlorothiophen-2-yl)-1,3,4-thiadiazol-2-yl)-4-(((1S,2R)-2-hydroxycyclopentyl)amino)-3-methoxy-2-oxo-2H-pyran-6-carboxamide (Compound 7a*) and N-(5-(3-chlorothiophen-2-yl)-1,3,4-thiadiazol-2-yl)-4-(((1R,2S)-2-hydroxycyclopentyl)amino)-3-methoxy-2-oxo-2H-pyran-6-carboxamide (Compound 7b*)

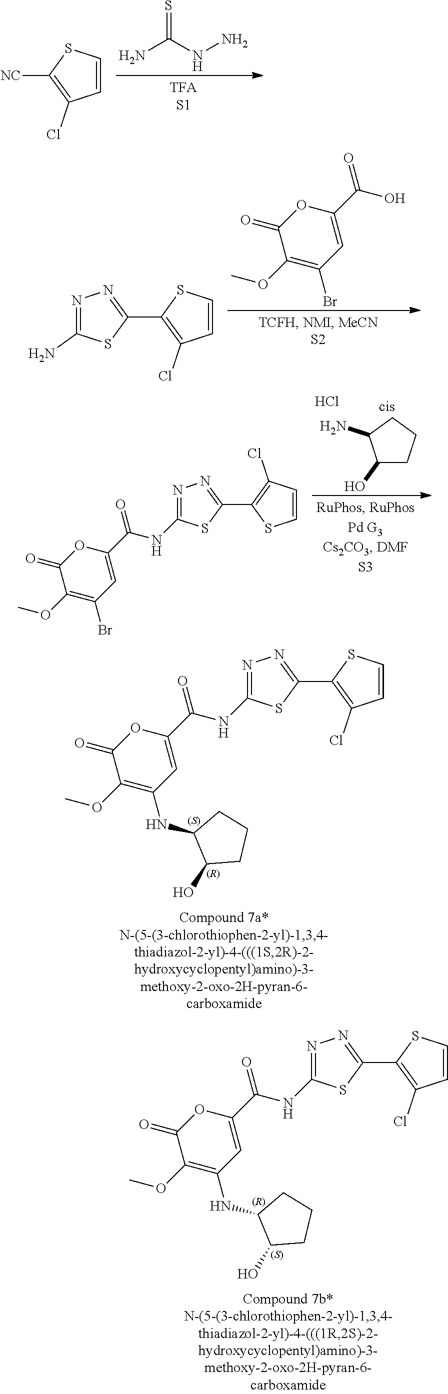

Steps 1-2: 4-Bromo-N-(5-(3-chlorothiophen-2-yl)-1,3,4-thiadiazol-2-yl)-3-methoxy-2-oxo-2H-pyran-6-carboxamide was prepared according to Example 4 Steps 1-2 using 3-chlorothiophene-2-carbonitrile instead of 4-chlorothiophene-3-carbonitrile. LCMS (ES, m/z)=448.0 [M+1]+.

Step 3: N-(5-(3-chlorothiophen-2-yl)-1,3,4-thiadiazol-2-yl)-4-(((1S,2R)-2-hydroxycyclopentyl)amino)-3-methoxy-2-oxo-2H-pyran-6-carboxamide (Compound 7a*) and N-(5-(3-chlorothiophen-2-yl)-1,3,4-thiadiazol-2-yl)-4-(((1R,2S)-2-hydroxycyclopentyl)amino)-3-methoxy-2-oxo-2H-pyran-6-carboxamide (Compound 7b*) were prepared as a racemic mixture according to Example 1, Part C, Step 2 using cis-2-aminocyclopentan-1-ol hydrochloride as the “amine reagent” and 4-bromo-N-(5-(3-chlorothiophen-2-yl)-1,3,4-thiadiazol-2-yl)-3-methoxy-2-oxo-2H-pyran-6-carboxamide as the “halo-pyrone reagent”. LCMS (ES, m/z)=468.85 [M+1]+. ¹H NMR (400 MHz, DMSO-d4) δ 13.47 (br, 1H), 7.93 (d, J=5.6 Hz, 1H), 7.43 (s, 1H), 7.32 (d, J=5.6 Hz, 1H), 6.25-6.18 (m, 1H), 5.20-5.09 (m, 1H), 4.11-4.07 (m, 1H), 3.97-3.81 (m, 1H), 3.74 (s, 3H), 2.08-1.98 (m, 1H), 1.92-1.71 (m, 2H), 1.71-1.44 (m, 3H).

### Example 6: (R)-4-((1-cyclopropyl-2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 21*) and (S)-4-((1-cyclopropyl-2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 22*)

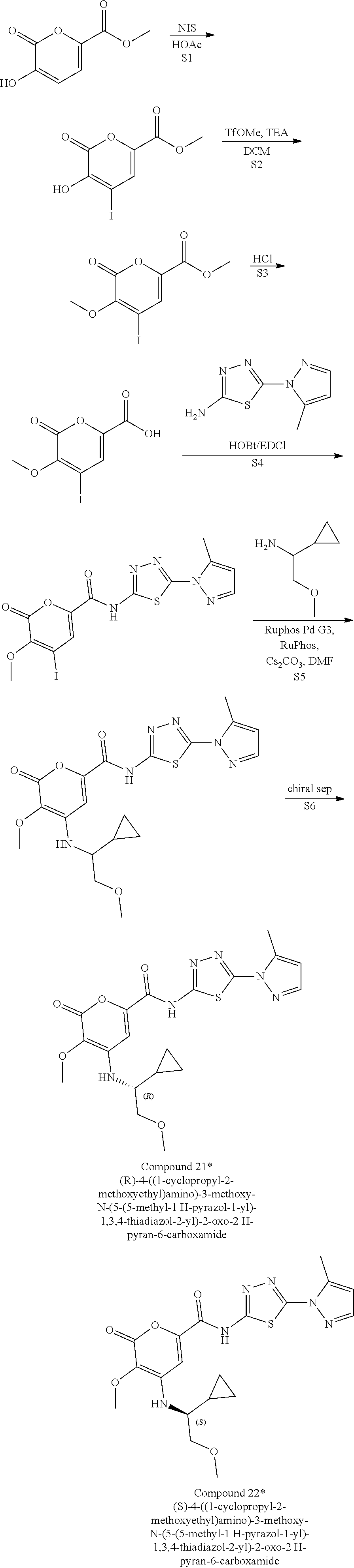

Step 1: Into a solution of methyl 5-hydroxy-6-oxopyran-2-carboxylate (25.0 g, 147 mmol, 1.00 equiv) in acetic acid (AcOH) (300 mL) was added N-iodosuccinimide (NIS) (39.0 g, 173 mmol, 1.18 equiv) in portions at room temperature. The resulting mixture was stirred for 20 h at 80° C. then concentrated under reduced pressure. The residue was dissolved in ethyl acetate (EtOAc) (1 L) and washed with water (3×100 mL), and dried over anhydrous Na₂SO₄. After filtration, the filtrate was concentrated under reduced pressure. The residue was purified using silica gel column chromatography, eluted with petroleum ether/ethyl acetate (PE/EtOAc) (3:2), to afford methyl 5-hydroxy-4-iodo-6-oxopyran-2-carboxylate (20.0 g, 46% yield). LCMS (ESI, m/z)=295 [M−1]⁻.

Step 2: Into a solution of methyl 5-hydroxy-4-iodo-6-oxopyran-2-carboxylate (20.0 g, 67.6 mmol, 1.00 equiv) in dichloromethane (DCM) (250 mL) was added diisopropylethylamine (DIEA) (26.0 g, 201 mmol, 2.98 equiv) at room temperature. To the above mixture was added triflate ester (33.0 g, 201 mmol, 2.98 equiv) dropwise at 0° C. The resulting mixture was stirred overnight at room temperature then poured into water and extracted with DCM (3×500 mL). The combined organic layers were washed with water (3×100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (4:1), to afford methyl 4-iodo-5-methoxy-6-oxopyran-2-carboxylate (19.0 g, 91% yield). LCMS (ESI, m/z)=311 [M+1]+.

Step 3: A solution of methyl 4-iodo-5-methoxy-6-oxopyran-2-carboxylate (5.0 g, 16 mmol, 1.0 equiv) in HCl (6M) (100 mL) was stirred for 4 h at 80° C. The resulting mixture was then concentrated under reduced pressure to provide 4-iodo-5-methoxy-6-oxopyran-2-carboxylic acid (3.8 g, 80/yield). LCMS (ESI, m/z)=295 [M−1]⁻.

Step 4: A solution of 4-iodo-5-methoxy-6-oxopyran-2-carboxylic acid (2.60 g, 8.78 mmol, 1.00 equiv), hydroxybenzotriazole (HOBT) (1.80 g, 13.3 mmol, 1.52 equiv), 1-ethyl-3-(3-dimethylaminopropyl)carbodiimide (EDCI) (3.60 g, 18.8 mmol, 2.14 equiv) and 5-(5-methylpyrazol-1-yl)-1,3,4-thiadiazol-2-amine (product of Example 1, Part B, Step 4) (1.40 g, 7.72 mmol, 0.88 equiv) in N,N-dimethylformamide (DMF) (40 mL) was stirred for 1 h at room temperature. The reaction was quenched by the addition of water (20 mL). The precipitated solids were collected by filtration and washed with acetonitrile (MeCN) (5×1 mL) to provide 4-iodo-3-methoxy-N-(5-(5-methyl-1H-pyrazol-1-yl)-1,3,4-thiadiazol-2-yl)-2-oxo-2H-pyran-6-carboxamide (also referred to herein as 4-iodo-5-methoxy-N-[5-(5-methylpyrazol-1-yl)-1,3,4-thiadiazol-2-yl]-6-oxopyran-2-carboxamide) (3.1 g, 77% yield). LCMS (EST, m/z)=460 [M+1]+.

Step 5: A mixture of 4-iodo-3-methoxy-N-(5-(5-methyl-1H-pyrazol-1-yl)-1,3,4-thiadiazol-2-yl)-2-oxo-2H-pyran-6-carboxamide as the “halo-pyrone reagent” (100 mg, 0.218 mmol, 1 equiv), (2-dicyclohexylphosphino-2′,6′-diisopropoxy-1,1′-biphenyl)[2-(2′-amino-1,1′-biphenyl)]palladium(II) methanesulfonate (RuPhos Palladacycle Gen3) (37 mg, 0.044 mmol, 0.20 equiv), (2-dicyclohexylphosphino-2′,6′-diisopropoxy-1,1′-biphenyl)[2-(2′-amino-1,1′-biphenyl) (RuPhos) (20 mg, 0.043 mmol, 0.20 equiv). Cs₂CO₃ (212 mg, 0.651 mmol, 2.99 equiv) and 1-cyclopropyl-2-methoxyethanamine as the “amine reagent” (25 mg, 0.217 mmol, 1.00 equiv) in N,N-dimethylformamide (DMF) (5.0 mL) was stirred for 2 h at 100° C. under nitrogen atmosphere. The mixture was allowed to cool down to room temperature. The residue was directly purified by reversed-phase flash chromatography (C18 silica gel; mobile phase, MeCN in water (10 mmol/L NH₄HCO₃), 10% to 50% gradient in 10 min; detector, UV 254 nm) and then dried in an oven under reduced pressure to give 140 mg of crude material. The crude material was further purified by Prep-HPLC with the following conditions (X Select CSH Prep C18 OBD Column, 19*250 mm; mobile phase A: water (10 mmol/L NH₄HCO₃). Mobile Phase B: acetonitrile (MeCN); flow rate: 20 mL/min; gradient: 25% B to 30% B in 8 min, 30% B; wave length: 254 nm; RT1 (min): 8), and then dried in an oven under reduced pressure to afford 70 mg of racemic 4-((1-cyclopropyl-2-methoxyethyl)amino)-3-methoxy-N-(5-(5-methyl-1H-pyrazol-1-yl)-1,3,4-thiadiazol-2-yl)-2-oxo-2H-pyran-6-carboxamide. Separation of its constituent enantiomers was performed by chiral-Prep-HPLC using the following conditions: (CHIRALPAK IF, 2*25 cm, 5 μm; Mobile Phase A: Hexanes (0.1% trifluoracetic acid (TFA)), Mobile Phase B: methanol:dichloromethane (methanol (MeOH):dichloromethane (DCM))=1:1; Flow rate: 20 mL/min; Gradient: 70% B to 70% B in 17 min; Wave Length: 254/220 nm; RT1 (min): 7.81; RT2 (min): 12.17; Sample Solvent: MeOH:DCM=1:1) provided two enantiomers with arbitrarily assigned stereochemistry: (R)-4-((1-cyclopropyl-2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 21*), first eluting peak, LCMS (ES, m/z)=447.16 [M+1]+, ¹H NMR (400 MHz, DMSO-d6) δ 7.78 (d, J=1.6 Hz, 1H), 7.33 (s, 1H), 6.44 (d, J=1.6 Hz, 1H), 3.68 (s, 3H), 3.53 (d, J=6.0 Hz, 2H), 3.38-3.33 (m, 1H), 3.25 (s, 3H), 2.67 (s, 3H), 1.06-1.02 (m, 1H), 0.54-0.48 (m, 1H), 0.46-0.42 (m, 1H), 0.39-0.30 (m, 2H); and (5)-4-((1-cyclopropyl-2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 22*), second eluting peak, LCMS (ES, m/z)=447.16 [M+1]+, ¹H NMR (400 MHz, DMSO-d6) δ 7.78 (d, J=1.6 Hz, 1H), 7.32 (s, 11H), 6.44 (d, J=1.6 Hz, 1H), 3.68 (s, 3H), 3.53 (d, J=5.6 Hz, 2H), 3.49-3.46 (m, 1H), 3.26 (s, 3H), 2.67 (s, 3H), 1.07-1.02 (m, 1H), 0.56-0.50 (m, 1H), 0.49-0.42 (m, 1H), 0.35-0.28 (m, 2H).

### Example 7: 4-(((cis)-2-hydroxycycloheptyl)amino)-3-methoxy-N-(5-(5-methyl-1H-pyrazol-1-yl)-1,3,4-thiadiazol-2-yl)-2-oxo-2H-pyran-6-carboxamide (Compound 26, Rac-26), 4-(((1S,2R)-2-hydroxycycloheptyl)amino)-3-methoxy-N-(5-(5-methyl-1H-pyrazol-1-yl)-1,3,4-thiadiazol-2-yl)-2-oxo-2H-pyran-6-carboxamide (Compound 26a*) and 4-(((1R,2S)-2-hydroxycycloheptyl)amino)-3-methoxy-N-(5-(5-methyl-1H-pyrazol-1-yl)-1,3,4-thiadiazol-2-yl)-2-oxo-2H-pyran-6-carboxamide (Compound 26b*)

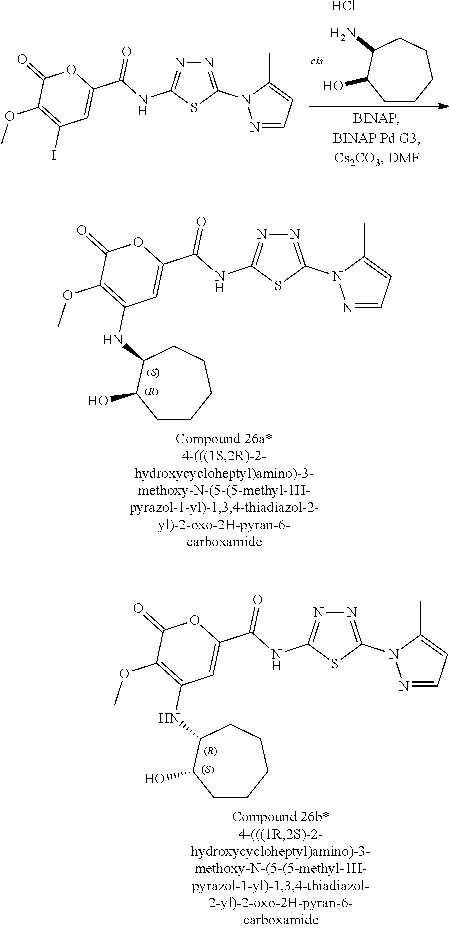

Into a stirred solution of 4-iodo-5-methoxy-N-[5-(5-methylpyrazol-1-yl)-1,3,4-thiadiazol-2-yl]-6-oxopyran-2-carboxamide (50 mg, 0.11 mmol, 1.00 equiv) (product of Step 4 of Example 6; “halo-pyrone reagent”) in N,N-dimethylformamide (DMF) (1.25 mL) were added (±)-2,2′-bis(diphenylphosphino)-1,1′-binaphthalene (rac-BINAP) (10 mg, 0.016 mmol, 0.15 equiv), rac-BINAP-Pd-G3 ([1-(2-diphenylphosphanylnaphthalen-1-yl)naphthalen-2-yl]-diphenylphosphane methanesulfonic acid, palladium, 2-phenylaniline) (15 mg, 0.015 mmol, 0.14 equiv), cesium carbonate (174 mg, 0.534 mmol, 4.90 equiv) and cis-2-aminocycloheptan-1-ol hydrochloride (20 mg, 0.121 mmol, 1.11 equiv) (“amine reagent”) at room temperature. The resulting mixture was stirred for 1 h at 100° C. under N₂ (nitrogen gas). The mixture was then directly purified by C18 reverse phase flash chromatography (acetonitrile (MeCN) in water (5 mmol/L NH₄HCO₃), 20% to 50% gradient over 15 min; detector, UV 254 nm). The crude product (20 mg) was then additionally purified by Prep-HPLC (XBridge Prep OBD C18 Column, 30*150 mm, 5 μm; mobile phase, water (10 mmol/L NH₄HCO₃) and acetonitrile (MeCN) (23% MeCN up to 31% in 8 min)) to provide a racemic mixture of the title compounds (Compound 26a* and Compound 26b*) (3.9 mg, 7.6% yield). LCMS (ES, m/z)=461.05 [M+1]+. ¹H NMR (400 MHz, DMSO-d6) δ 7.76 (d, J=1.6 Hz, 1H), 7.24 (s, 1H), 6.42 (d, J=1.6 Hz, 1H), 6.22 (br, 1H), 5.10-5.02 (m, 1H), 3.95-3.90 (m, 1H), 3.78-3.66 (m, 1H), 3.65 (s, 3H), 2.67 (s, 3H), 1.96-1.61 (m, 6H), 1.61-1.33 (m, 4H).

### Example 8: 4-(((cis)-2-hydroxy-2,3-dihydro-1H-inden-1-yl)amino)-3-methoxy-N-(5-(5-methyl-1H-pyrazol-1-yl)-1,3,4-thiadiazol-2-yl)-2-oxo-2H-pyran-6-carboxamide (Compound 28, Rac-28), 4-(((1R,2S)-2-hydroxy-2,3-dihydro-1H-inden-1-yl)amino)-3-methoxy-N-(5-(5-methyl-1H-pyrazol-1-yl)-1,3,4-thiadiazol-2-yl)-2-oxo-2H-pyran-6-carboxamide (Compound 28a*) and 4-(((1S,2R)-2-hydroxy-2,3-dihydro-1H-inden-1-yl)amino)-3-methoxy-N-(5-(5-methyl-1H-pyrazol-1-yl)-1,3,4-thiadiazol-2-yl)-2-oxo-2H-pyran-6-carboxamide (Compound 28b*)

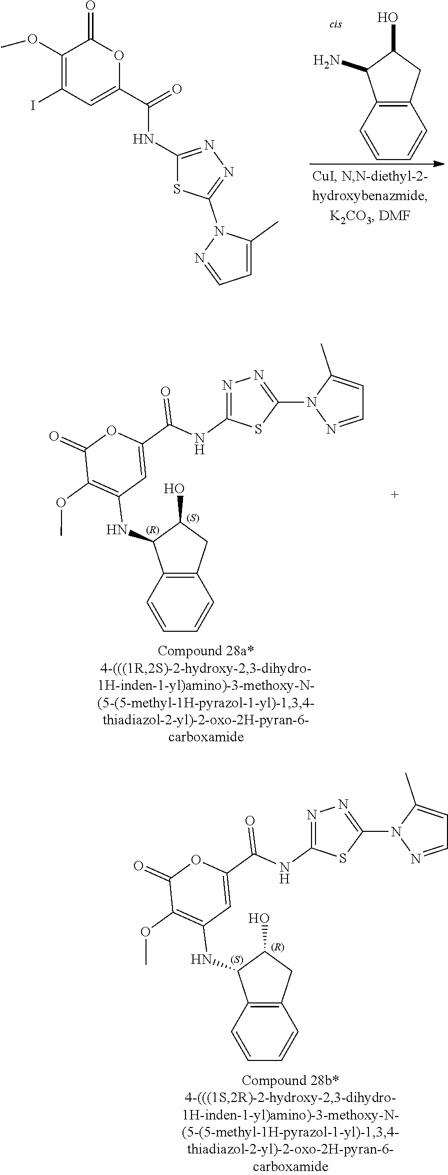

To a stirred solution of 4-iodo-5-methoxy-N-[5-(5-methylpyrazol-1-yl)-1,3,4-thiadiazol-2-yl]-6-oxopyran-2-carboxamide (product of Step 4 of Example 6) (200 mg, 0.436 mmol, 1 equiv) (“halo-pyrone reagent”) in N,N-dimethylformamide (DMF) (2.5 mL) was added CuI (20 mg, 0.105 mmol, 0.24 equiv), N,N-diethyl-2-hydroxybenzamide (20 mg, 0.103 mmol, 0.24 equiv), K₂CO₃ (120 mg, 0.868 mmol, 1.99 equiv) and cis-1-amino-2,3-dihydro-1H-inden-2-ol (80 mg, 0.536 mmol, 1.23 equiv) (“amine reagent”) at room temperature. The resulting mixture was stirred for 40 mins at 80° C. under N₂ (nitrogen gas). The mixture was then directly purified by C18 reverse phase flash chromatography (acetonitrile (MeCN) in water (5 mmol/L NH₄HCO₃), 10% to 50% gradient in 10 min; detector, UV 254 nm). The crude product (40 mg) was then further purified by Prep-HPLC (Xselect CSH C18 OBD Column 30*150 mm 5 um; mobile phase, water (0.05% trifluoroacetic acid (TFA)) and acetonitrile (MeCN) (31% MeCN up to 41% in 10 min)) to afford a racemic mixture of 4-(((1R,2S)-2-hydroxy-2,3-dihydro-1H-inden-yl)amino)-3-methoxy-N-(5-(5-methyl-1H-pyrazol-1-yl)-1,3,4-thiadiazol-2-yl)-2-oxo-2H-pyran-6-carboxamide (Compound 28a*) and 4-(((1S,2R)-2-hydroxy-2,3-dihydro-1H-inden-1-yl)amino)-3-methoxy-N-(5-(5-methyl-H-pyrazol-1-yl)-1,3,4-thiadiazol-2-yl)-2-oxo-2H-pyran-6-carboxamide (Compound 28b*) (3.4 mg, 1.6% yield). LCMS (ES, m/z)=481.05 [M+1]+. ¹H NMR (400 MHz, DMSO-d6) δ 11.42 (br, 1H), 7.89 (d, J=8.8 Hz, 1H), 7.83 (d, J=1.6 Hz, 1H), 7.28-7.19 (m, 4H), 7.10 (s, 1H), 6.48 (d, J=1.6 Hz, 1H), 5.41-5.25 (m, 2H), 4.54-4.96 (m, 1H), 3.39 (s, 3H), 3.16-3.09 (m, 1H), 2.90-2.82 (m, 1H), 2.71 (s, 3H).

### Example 9: 4-((1,1-dioxidotetrahydro-2H-thiopyran-4-yl)amino)-3-methoxy-N-(5-(5-methyl-1H-pyrazol-1-yl)-1,3,4-thiadiazol-2-yl)-2-oxo-2H-pyran-6-carboxamide (Compound 29)

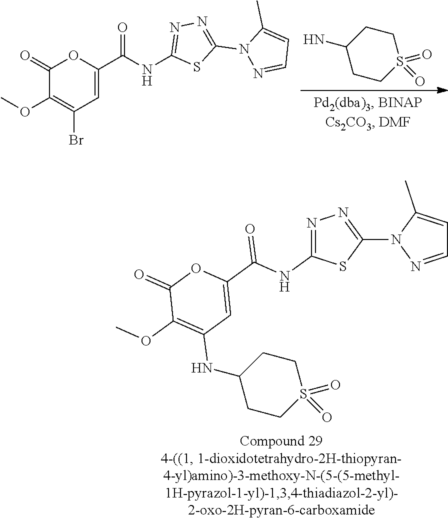

To a stirred solution of 4-bromo-5-methoxy-N-[5-(5-methylpyrazol-1-yl)-1,3,4-thiadiazol-2-yl]-6-oxopyran-2-carboxamide (product of Example 1, Part C, Step 1; “halo-pyrone reagent”) (100 mg, 0.24 mmol, 1.00 equiv) (“halo-pyrone reagent”), 4-aminotetrahydro-2H-thiopyran 1,1-dioxide (125 mg, 0.84 mmol, 3.45 equiv) (“amine reagent”) in N,N-dimethylformamide (DMF) (5 mL) was added tris(dibenzylidenaceton)dipalladium(0) dibenzylidenacetone (Pd₂(dba)₃) (20 mg, 0.04 mmol, 0.14 equiv), (±)-2,2′-bis(diphenylphosphino)-1,1′-binaphthalene (rac-BINAP) (32 mg, 0.05 mmol, 0.21 equiv), cesium carbonate (240 mg, 0.74 mmol, 3.04 equiv) at room temperature under N₂ (nitrogen gas). The resulting mixture was stirred at 120° C. for 2 h under N₂. The resulting mixture was diluted with water (40 mL) and extracted with ethyl acetate (EtOAc) (3×50 mL). The organic extracts were concentrated under reduced pressure and the residue was purified by Prep-HPLC (XSelect CSH Prep C18 OBD Column, 19*250 mm, 5 μm; Mobile Phase A: water (0.05% trifluoroacetic acid (TFA)), Mobile Phase B: methanol (MeOH); Flow rate: 20 mL/min; Gradient: 53% B to 58% B in 8 min, 58% B; Wave Length: 254 nm) to afford 4-(0,1-dioxidotetrahydro-2H-thiopyran-4-yl)amino)-3-methoxy-N-(5-(5-methyl-1H-pyrazol-1-yl)-1,3,4-thiadiazol-2-yl)-2-oxo-2H-pyran-6-carboxamide (Compound 29) (10.6 mg, 9.0% yield). LCMS (ESI, m/z)=[M+1]+=481.1. ¹H NMR (400 MHz, CD₃OD) δ 7.68 (d, J=1.6 Hz, 1H), 7.40 (s, 1H), 6.35 (d, J=1.6 Hz, 1H), 4.10-4.02 (m, 1H), 3.83 (s, 3H), 3.40-3.35 (m, 4H), 2.74 (s, 3H), 2.35-2.26 (m, 4H).

### Example 10: N-(5-(3-chloro-1-methyl-1H-pyrrol-2-yl)-1,3,4-thiadiazol-2-yl)-4-(isopropylamino)-3-methoxy-2-oxo-2H-pyran-6-carboxamide (Compound 131)

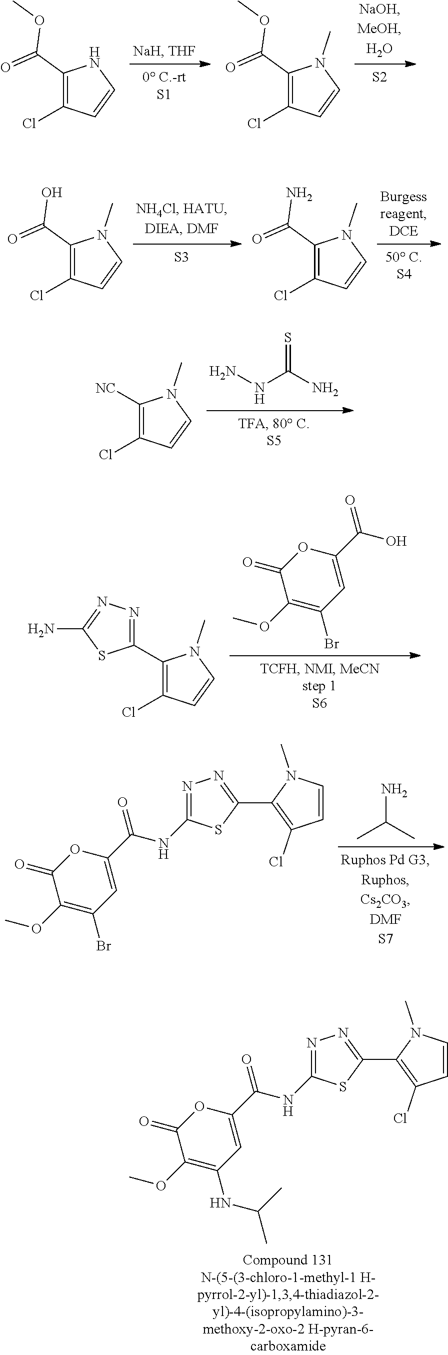

Step 1: Into a 250 mL round-bottom flask were added methyl 3-chloro-1H-pyrrole-2-carboxylate (2.40 g, 15.0 mmol, 1.00 equiv) and tetrahydrofuran (THF) (20 mL, 247 mmol, 16.4 equiv) at room temperature. To the above mixture was added NaH (1.44 g, 60.0 mmol, 3.99 equiv) in portions at 0° C. The resulting mixture was stirred for an additional 1 h at 0° C. To the above mixture was then added methyl iodide (MeI) (6.48 g, 45.6 mmol, 3.04 equiv) dropwise at 0° C. The resulting mixture was then stirred overnight at room temperature. The reaction was then quenched by the addition of HCl (1N) (40 mL) at 0° C. The resulting mixture was extracted with ethyl acetate (EtOAc) (3×40 mL). The combined organic layers were washed with water (2×20 mL), dried over anhydrous Na₂SO₄. After filtration, the filtrate was concentrated under reduced pressure. The residue was purified by silica gel column chromatography, eluting with petroleum ether/ethyl acetate (PE/EtOAc) (1:1) to afford methyl 3-chloro-1-methyl-1H-pyrrole-2-carboxylate (1.7 g, 71% yield). LCMS (ESI, m/z)=173.85 [M+1]+.

Step 2: Into a 40 mL vial was added methyl 3-chloro-1-methyl-1H-pyrrole-2-carboxylate (750 mg, 4.32 mmol, 1.00 equiv) and methanol (MeOH) (4.0 mL, 99 mmol, 23 equiv) at room temperature. To the above mixture was added NaOH (330 mg, 8.25 mmol, 1.91 equiv) in H₂O (4.0 mL, 222 mmol, 51.4 equiv) at room temperature. The resulting mixture was stirred for an additional 2 h at 50° C. The mixture was then acidified to pH 6 with HCl (3 M). The resulting mixture was extracted with ethyl acetate (EtOAc) (3×30 mL). The combined organic layers were washed with water (2×10 mL), dried over anhydrous Na₂SO₄. After filtration, the filtrate was concentrated under reduced pressure to afford 3-chloro-1-methylpyrrole-2-carboxylic acid (580 mg, 80% yield). LCMS (ESI, m/z)=160.00 [M+1]+.

Step 3: Into a 40 mL vial was added 3-chloro-1-methylpyrrole-2-carboxylic acid (2.20 g, 13.8 mmol, 1.00 equiv), N,N-dimethylformamide (DMF) (20 mL), 1-[Bis(dimethylamino)methylene]-1H-1,2,3-triazolo[4,5-b]pyridinium 3-oxid hexafluorophosphate (15.7 g, 41.4 mmol, 3.00 equiv), diisopropylethylamine (DIEA) (5.42 g, 41.9 mmol, 3.04 equiv) and NH₄Cl (2.97 g, 55.6 mmol, 4.03 equiv) at room temperature. The resulting mixture was stirred for 3 h at 80° C. The reaction was then quenched with water at room temperature and the aqueous layer was extracted with ethyl acetate (EtOAc) (3×30 mL). The combined organic layers were washed with brine (3×30 mL) then concentrated under reduced pressure. The resulting residue was purified by C18 reverse phase flash chromatography (acetonitrile (MeCN) in water, 20% to 50% gradient in 10 min; detector, UV 254 nm) to afford 3-chloro-1-methylpyrrole-2-carboxamide (1.47 g, 60% yield). LCMS (ESI, m/z)=159.05 [M+1]+.

Step 4: Into a 40 mL vial was added 3-chloro-1-methylpyrrole-2-carboxamide (1.40 g, 8.83 mmol, 1.00 equiv), dichloroethane (DCE) (20 mL) and methyl N-(triethylammoniumsulfonyl)carbamate (Burgess reagent) (6.29 g, 26.4 mmol, 2.99 equiv) at room temperature. The resulting mixture was stirred for 2 h at 50° C. The reaction was then quenched with water at room temperature and extracted with dichloromethane (DCM) (3×20 mL). The combined organic layers were washed with water (3×20 mL), dried over anhydrous Na₂SO₄. After filtration, the filtrate was concentrated under reduced pressure and the residue purified by silica gel column chromatography, eluted with petroleum ether/ethyl acetate (PE/EtOAc) (70:30) to afford 3-chloro-1-methylpyrrole-2-carbonitrile (940 mg, 68% yield). ¹H NMR (400 MHz, DMSO-d6) δ 7.24 (d, J=2.8 Hz, 1H), 6.34 (d, J=2.8 Hz, 1H), 3.74 (s, 3H).

Step 5: A mixture of 3-chloro-1-methylpyrrole-2-carbonitrile (100 mg, 0.71 mmol, 1.00 equiv) and thiosemicarbazide (200 mg, 2.19 mmol, 3.09 equiv) in trifluoroacetic acid (TFA) (5.00 mL, 67.3 mmol, 94.6 equiv) was stirred for 16 h at 80° C. The resulting mixture was concentrated under reduced pressure and the residue was purified by C18 reverse phase flash chromatography (acetonitrile (MeCN) in water, 30% to 40% gradient in 10 min; detector, UV 254 nm) to afford 5-(3-chloro-1-methylpyrrol-2-yl)-1,3,4-thiadiazol-2-amine (45 mg, 26% yield). LCMS (EST, m/z)=215.00 [M+1]+.

Step 6: Into a solution of 4-bromo-5-methoxy-6-oxopyran-2-carboxylic acid (product of Example 1, Part A, Step 4) (770 mg, 3.09 mmol, 1.00 equiv) in acetonitrile (MeCN) (20 mL) was added chloro-N,N,N′,N′-tetramethylformamidinium hexafluorophosphate (TCFH) (960 mg, 3.42 mmol, 1.11 equiv), N-methylimidazole (NMI) (900 mg, 11.0 mmol, 3.54 equiv) and 5-(3-chloro-1-methylpyrrol-2-yl)-1,3,4-thiadiazol-2-amine (616 mg, 2.87 mmol, 0.93 equiv) at room temperature. The resulting mixture was stirred for 1 h at room temperature. The precipitated solids were collected by filtration and washed with acetonitrile (3×10 mL) to afford 4-bromo-N-[5-(3-chloro-1-methylpyrrol-2-yl)-1,3,4-thiadiazol-2-yl]-5-methoxy-6-oxopyran-2-carboxamide (also referred to herein as 4-bromo-N-(5-(3-chloro-1-methyl-1H-pyrrol-2-yl)-1,3,4-thiadiazol-2-yl)-3-methoxy-2-oxo-2H-pyran-6-carboxamide) (910 mg, 65% yield). ¹H NMR (400 MHz, DMSO-d6) δ 13.67 (s, 1H), 7.66 (s, 1H), 7.18 (d, J=2.8 Hz, 1H), 6.34 (d, J=2.8 Hz, 1H), 4.00 (s, 3H), 3.95 (s, 3H).

Step 7: N-(5-(3-Chloro-1-methyl-1H-pyrrol-2-yl)-1,3,4-thiadiazol-2-yl)-4-(isopropylamino)-3-methoxy-2-oxo-2H-pyran-6-carboxamide (Compound 131) was prepared according to Example 1, Part C, Step 2 using 4-bromo-N-[5-(3-chloro-1-methylpyrrol-2-yl)-1,3,4-thiadiazol-2-yl]-5-methoxy-6-oxopyran-2-carboxamide as the “halo-pyrone reagent” and propan-2-amine as the “amine reagent”. LCMS (ES, m/z)=424.05 [M+1]+. ¹H NMR (400 MHz, DMSO-d6) δ 7.30 (s, 1H), 7.13 (d, J=2.8 Hz, 1H), 6.70 (d, J=8.8 Hz, 1H), 6.31 (d, J=2.8 Hz, 1H), 3.94 (s, 3H), 3.91-3.84 (m, 1H), 3.68 (s, 3H), 1.23 (d, J=5.6 Hz, 6H).

### Example 11: N-(5-(3-chloro-1-methyl-1H-pyrrol-2-yl)-1,3,4-thiadiazol-2-yl)-4-(((1S,2R)-2-hydroxycyclobutyl)amino)-3-methoxy-2-oxo-2H-pyran-6-carboxamide (Compound 53*) and N-(5-(3-chloro-1-methyl-1H-pyrrol-2-yl)-1,3,4-thiadiazol-2-yl)-4-(((1R,2S)-2-hydroxycyclobutyl)amino)-3-methoxy-2-oxo-2H-pyran-6-carboxamide (Compound 54*)

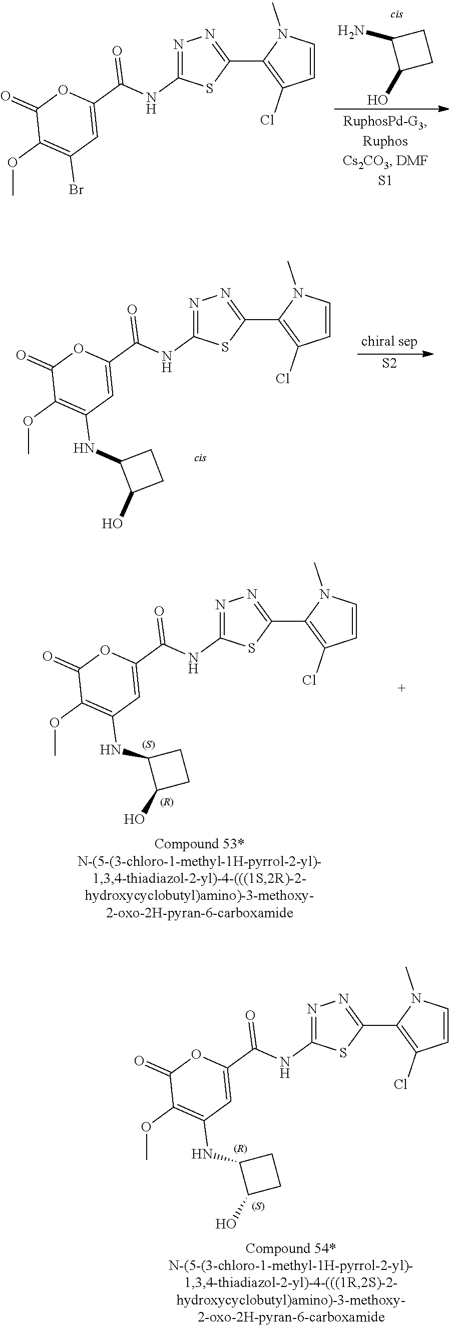

Racemic N-(5-(3-chloro-1-methyl-1H-pyrrol-2-yl)-1,3,4-thiadiazol-2-yl)-4-(((1,2-cis)-2-hydroxycyclobutyl)amino)-3-methoxy-2-oxo-2H-pyran-6-carboxamide prepared according to Example 1, Part C, Step 2 using 4-bromo-N-(5-(3-chloro-1-methyl-1H-pyrrol-2-yl)-1,3,4-thiadiazol-2-yl)-3-methoxy-2-oxo-2H-pyran-6-carboxamide (product of Example 10, Step 6) as the “halo-pyrone reagent” and (1,2-cis)-2-aminocyclobutan-1-ol as the “amine reagent”. Separation of constituent enantiomers of racemic N-(5-(3-chloro-1-methyl-1H-pyrrol-2-yl)-1,3,4-thiadiazol-2-yl)-4-(((1,2-cis)-2-hydroxycyclobutyl)amino)-3-methoxy-2-oxo-2H-pyran-6-carboxamide by Prep-Chiral-HPLC (condition: Column: CHIRAL ART Cellulose-SC, 2*25 cm, 5 μm; Mobile Phase A: Hexanes (0.1% trifluoroacetic acid (TFA)). Mobile Phase B: methanol:dichloromethane (MeOH/DCM)=1:1; Flow rate: 20 mL/min; Gradient: 50% B to 50% B in 21.5 min; Wave Length: 220/254 nm; RT1 (min): 10.66; RT2 (min): 13.55; Sample Solvent: MeOH:DCM=1:1) provided two enantiomers with arbitrarily assigned stereochemistry: N-(5-(3-chloro-1-methyl-1H-pyrrol-2-yl)-1,3,4-thiadiazol-2-yl)-4-(((1S,2R)-2-hydroxycyclobutyl)amino)-3-methoxy-2-oxo-2H-pyran-6-carboxamide (Compound 53′), first eluting peak, LCMS (ES, m/z)=452.0 [M+1]+. ¹H NMR (400 MHz, DMSO-d₆) 7.23-7.14 (m, 2H), 6.59 (br, 1H), 6.34 (d, J=2.8 Hz, 1H), 5.50-5.43 (d, J=5.6 Hz, 1H), 4.42-4.34 (t, J=3.6 Hz, 1H), 4.21-4.11 (d, 1H), 3.95 (s, 3H), 3.75 (s, 3H), 2.21-2.06 (m, 2H), 2.01-1.91 (m, 1H), 1.89-1.88 (m, 1H); and N-(5-(3-chloro-1-methyl-1H-pyrrol-2-yl)-1,3,4-thiadiazol-2-yl)-4-(((1R,2S)-2-hydroxycyclobutyl)amino)-3-methoxy-2-oxo-2H-pyran-6-carboxamide (Compound 54*), second eluting peak, LCMS (ES, m/z)=452.0 [M+1]+, ¹H NMR (400 MHz, DMSO-d₆) 7.23-7.14 (m, 2H), 6.59 (br, 1H), 6.34 (d, J=2.8 Hz, 1H), 5.50-5.43 (d, J=5.6 Hz, 1H), 4.42-4.34 (t, J=3.6 Hz, 1H), 4.21-4.11 (d, 1H), 3.95 (s, 3H), 3.75 (s, 3H), 2.21-2.06 (m, 2H), 2.01-1.91 (m, 1H), 1.89-1.88 (m, 1H).

### Example 12: 4-(((1R,2S)-2-cyanocyclopentyl)amino)-3-methoxy-N-(5-(5-methyl-1H-pyrazol-1-yl)-1,3,4-thiadiazol-2-yl)-2-oxo-2H-pyran-6-carboxamide (Compound 73*) and 4-(((1S,2R)-2-cyanocyclopentyl)amino)-3-methoxy-N-(5-(5-methyl-1H-pyrazol-yl)-1,3,4-thiadiazol-2-yl)-2-oxo-2H-pyran-6-carboxamide (Compound 74*)

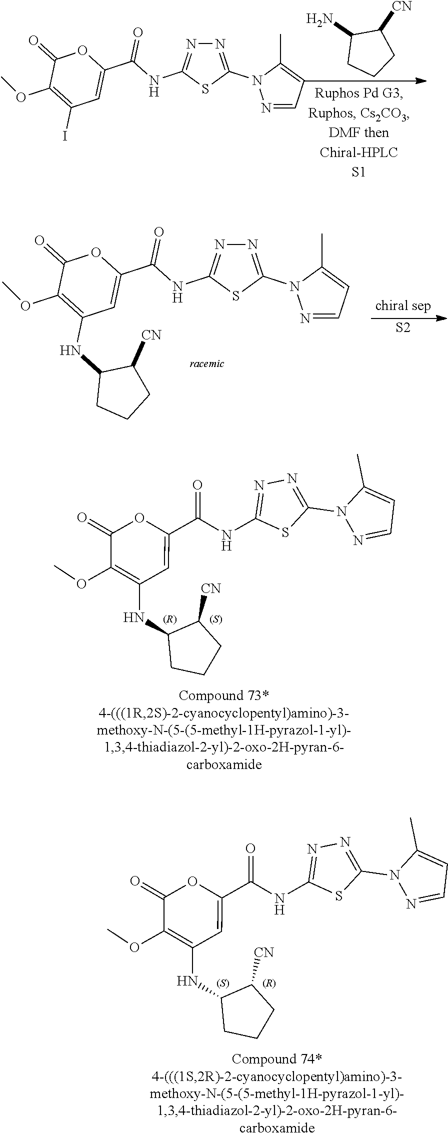

Racemic 4-(((1,2-cis)-2-cyanocyclopentyl)amino)-3-methoxy-N-(5-(5-methyl-1H-pyrazol-1-yl)-1,3,4-thiadiazol-2-yl)-2-oxo-2H-pyran-6-carboxamide was prepared according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and (1,2-cis)-2-aminocyclopentane-1-carbonitrile 2,2,2-trifluoroacetate as the “amine reagent”. Separation of constituent enantiomers of racemic 4-(((1,2-cis)-2-cyanocyclopentyl)amino)-3-methoxy-N-(5-(5-methyl-1H-pyrazol-1-yl)-1,3,4-thiadiazol-2-yl)-2-oxo-2H-pyran-6-carboxamide by Prep-Chiral-HPLC (Column: CHIRALPAK ID, 2*25 cm, 5 su; Mobile Phase A: Hexanes (0.1% trifluoroacetic acid (TFA)), Mobile Phase B: methanol (MeOH):dichloromethane (DCM)=1:1; Flow rate: 20 mL/min; Gradient: 90% B to 90% B in 17 min; Wave Length: 220/254 nm; RT1 (min); 6.85; RT2 (min): 10.82; Sample Solvent: MeOH:DCM=1:1) provided two enantiomers with arbitrarily assigned stereochemistry: 4-(((1R,2S)-2-cyanocyclopentyl)amino)-3-methoxy-N-(5-(5-methyl-1H-pyrazol-1-yl)-1,3,4-thiadiazol-2-yl)-2-oxo-2H-pyran-6-carboxamide (Compound 73*), first eluting peak, LCMS (ES, m/z)=442.1 [M+1]+, ¹HNMR (400 MHz, DMSO-d6) δ 13.35 (s, 1H), 7.79 (s, 1H), 7.45 (s, 1H), 7.19 (d, J=8.8 Hz, 1H), 6.45 (s, 1H), 4.43-4.38 (m, 1H), 3.72 (s, 3H), 3.46-3.40 (m, 1H), 2.68 (s, 3H), 2.14-2.03 (m, 2H), 2.02-1.91 (m, 2H), 1.62-1.46 (m, 1H), 1.61-1.47 (m, 1H); and 4-(((1S,2R)-2-cyanocyclopentyl)amino)-3-methoxy-N-(5-(5-methyl-1H-pyrazol-1-yl)-1,3,4-thiadiazol-2-yl)-2-oxo-2H-pyran-6-carboxamide (Compound 74*), second eluting peak, LCMS (ES, m/z)=442.10 [M+1]+, ¹H NMR (400 MHz, DMSO-d&) δ 13.35 (s, 1H), 7.79 (s, 1H), 7.45 (s, 1H), 7.19 (d, J=8.8 Hz, 1H), 6.45 (s, 1H), 4.43-4.38 (m, 1H), 3.72 (s, 3H), 3.46-3.40 (m, 1H), 2.68 (s, 3H), 2.14-2.03 (m, 2H), 2.02-1.91 (m, 2H), 1.62-1.46 (m, 1H), 1.61-1.47 (m, 1H).

### Example 13: 3-methoxy-4-((2-methoxyethyl)amino)-N-(5-(5-methyl-1H-pyrazol-1-yl)-1,3,4-thiadiazol-2-yl)-2-oxo-2H-pyran-6-carboxamide (Compound 75)

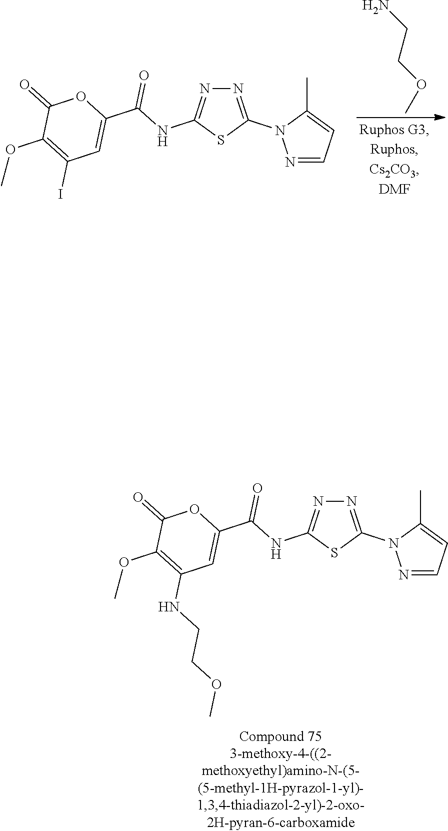

3-Methoxy-4-((2-methoxy ethyl)amino)-N-(5-(5-methyl-1H-pyrazol-1-yl)-1,3,4-thiadiazol-2-yl)-2-oxo-2H-pyran-6-carboxamide (Compound 75) was prepared according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and 2-methoxyethan-1-amine as the “amine reagent”. LCMS (ES, m/z)=407.05 [M+1]+. ¹H NMR (400 MHz, DMSO-d₆) δ 13.31 (s, 1H), 7.78 (d, J=1.6 Hz, 1H), 7.39 (s, 1H), 7.06 (br, 1H), 6.44 (d, J=1.6 Hz, 1H), 3.69 (s, 3H), 3.52-3.48 (m, 4H), 3.29 (s, 3H), 2.51 (s, 3H).

### Example 14: 3-methoxy-4-(((1S,2R)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 79*) and 3-methoxy-4-(((1R,2S)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 80*)

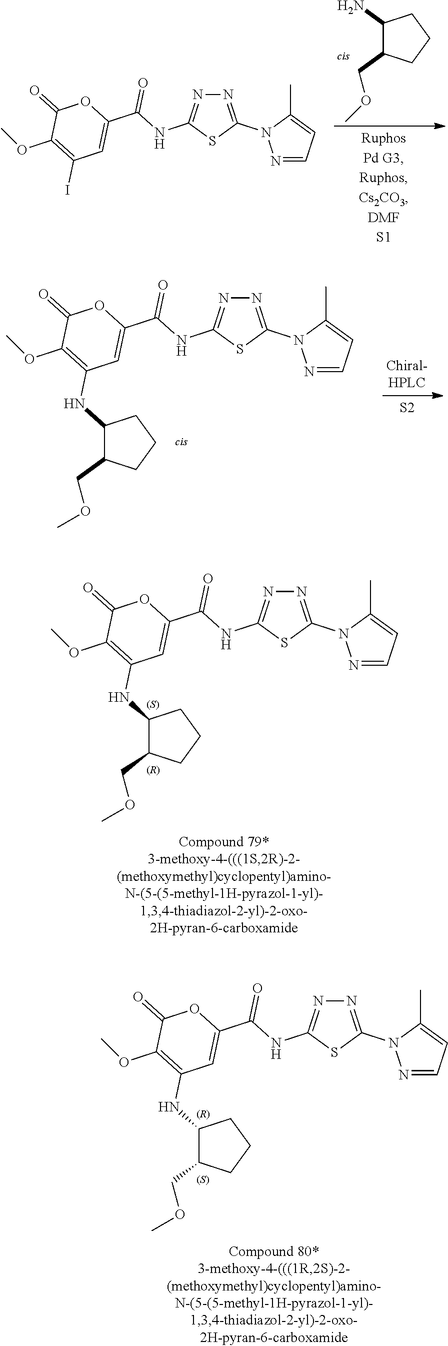

Racemic 3-methoxy-4-(((1,2-cis)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and cis-2-(methoxymethyl)cyclopentanamine as the “amine reagent”. Separation of constituent enantiomers of racemic 3-methoxy-4-(((1,2-cis)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide by Prep-Chiral-HPLC (conditions: CHIRAL ART Amylose-SA, 2*25 cm, 5 μm; Mobile Phase A: Hexanes (0.1% trifluoroacetic acid (TFA)), Mobile Phase B: ethanol (EtOH):dichloromethane (DCM)=1:1; Flow rate: 20 mL/min; Gradient: 50/B to 50% B in 9.5 min; RT1 (min): 7.14; RT2 (min): 8.71; Sample Solvent: ethanol/dichloromethane (EtOH:DCM)=1:1) provided two enantiomers with arbitrarily assigned stereochemistry: 3-methoxy-4-(((1S,2R)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 79*), first eluting peak, LCMS (ES, m/z)=461.10 [M+1]+, ¹H NMR (400 MHz, DMSO-d₆) δ 7.78 (d, J=1.6 Hz, 1H), 7.41 (s, 1H), 6.73 (d, J=8.4 Hz, 1H), 6.43 (d, J=1.6 Hz, 1H), 4.22-4.12 (m, 1H), 3.71 (s, 3H), 3.31 (s, 2H), 3.18 (s, 3H), 2.68 (s, 3H), 2.39-2.28 (m, 1H), 2.07-1.99 (m, 1H), 1.83-1.62 (m, 2H), 1.58-1.44 (m, 2H); and 3-methoxy-4-(((1R,2S)-2-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 80*), second eluting peak. LCMS (ES, m/z)=461.10 [M+1]+, ¹H NMR (400 MHz, DMSO-4) δ 7.78 (d, J=1.6 Hz, 1H), 7.41 (s, 1H), 6.73 (d, J=8.4 Hz, 1H), 6.43 (d, J=1.6 Hz, 1H), 4.22-4.12 (m, 1H), 3.71 (s, 3H), 3.31 (s, 2H). 3.18 (s, 3H), 2.68 (s, 3H), 2.39-2.28 (m, 1H), 2.07-1.99 (m, 1H), 1.83-1.62 (m, 2H), 1.58-1.44 (m, 2H).

### Example 15: (R)-4-((2-methoxy-1-phenylethyl)amino)-3-(2-methoxyethoxy)-N-(5-(S-methyl-1H-pyrazol-1-yl)-1,3,4-thiadiazol-2-yl)-2-oxo-2H-pyran-6-carboxamide (Compound 200)

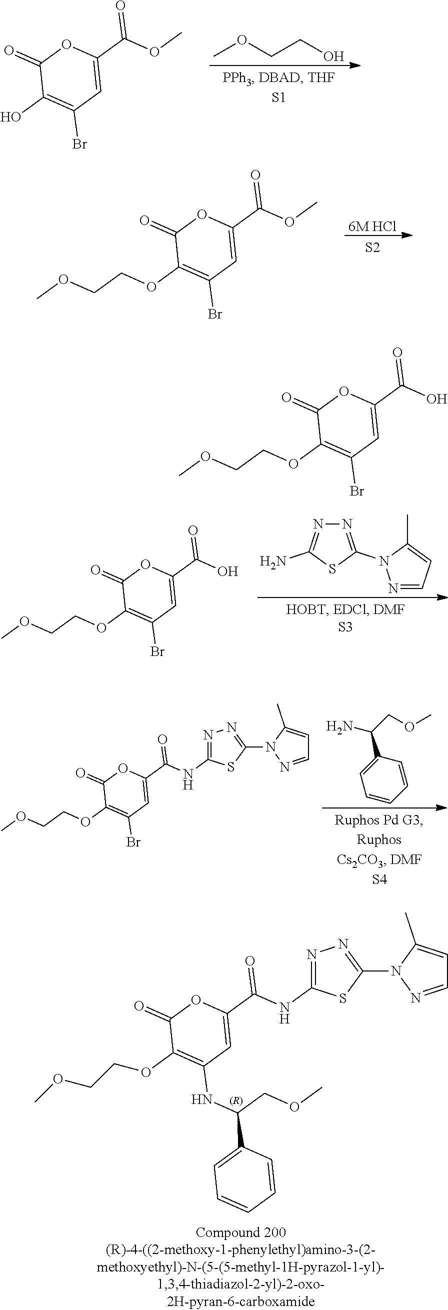

Step 1: To a stirred solution of methyl 4-bromo-5-hydroxy-6-oxopyran-2-carboxylate (product of Example 1, Part A, Step 2) (5.0 g, 20 mmol, 1.0 equiv) and 2-methoxyethanol (2.0 g, 26 mmol, 1.3 equiv) in tetrahydrofuran (THF) was added triphenyl phosphine (PPh₃) (8.0 g, 30 mmol, 1.5 equiv) in portions at room temperature. The resulting mixture was stirred for 10 min at 0° C. To this was then added di-tert-butyl azodicarboxylate (DBAD) (7.0 g, 30 mmol, 1.5 equiv) dropwise at room temperature. The resulting mixture was stirred overnight at room temperature. The mixture was then diluted with ethyl acetate (EtOAc) (600 mL), washed with water (3×200 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (1:3), to afford methyl 4-bromo-5-(2-methoxyethoxy)-6-oxopyran-2-carboxylate (3.7 g, 60% yield). LCMS (ESI, m/z)=307.309 [M+1]+.

Step 2: A solution of methyl 4-bromo-5-(2-methoxyethoxy)-6-oxopyran-2-carboxylate (1700 mg, 5.54 mmol, 1.00 equiv) in HCl (6M) (30 mL) was stirred for 3 h at 80° C. The resulting mixture was then concentrated under reduced pressure and diluted with ethyl acetate (EtOAc) (20) mL), washed with brine (3×10 mL), and dried over anhydrous Na₂SO₄. After filtration, the filtrate was concentrated under reduced pressure to provide 4-bromo-5-(2-methoxyethoxy)-6-oxopyran-2-carboxylic acid which was used directly in the next step without further purification. LCMS (ESI, m/z)=293.0 [M+1]+.

Step 3: To a stirred solution of 4-bromo-5-(2-methoxyethoxy)-6-oxopyran-2-carboxylic acid (1200 mg, 4.09 mmol, 1.00 equiv) and 5-(5-methylpyrazol-1-yl)-1,3,4-thiadiazol-2-amine (also referred to as 5-(5-methyl-1H-pyrazol-1-yl)-1,3,4-thiadiazol-2-amine) (product of Example 1, Part B, Step 4) (820 mg, 4.52 mmol, 1.11 equiv) in N,N-dimethylformamide (DMF) (21 mL) was added hydroxybenzotriazole (HOBT) (0110 mg, 8.22 mmol, 2.01 equiv) and 1-ethyl-3-(3-dimethylaminopropyl)carbodiimide (EDCI) (2355 mg, 12.3 mmol, 3.00 equiv) at room temperature. The resulting mixture was stirred for 1 h at room temperature before being diluted with water (40 mL). The precipitated solids were collected by filtration to afford 4-bromo-3-(2-methoxyethoxy)-N-(5-(5-methyl-1H-pyrazol-1-yl)-1,3,4-thiadiazol-2-yl)-2-oxo-2H-pyran-6-carboxamide (also referred to herein as 4-bromo-5-(2-methoxyethoxy)-N-[5-(5-methylpyrazol-1-yl)-1,3,4-thiadiazol-2-yl]-6-oxopyran-2-carboxamide) (1.3 g, 70% yield). LCMS (ESI, m/z)=458.05 [M+1]+.

Step 4: (R)-4-((2-Methoxy-1-phenylethyl)amino)-3-(2-methoxyethoxy)-N-(5-(5-methyl-1H-pyrazol-1-yl)-1,3,4-thiadiazol-2-yl)-2-oxo-2H-pyran-6-carboxamide (Compound 200) was prepared according to Example 1, Part C, Step 2 using 4-bromo-3-(2-methoxyethoxy)-N-(5-(5-methyl-1H-pyrazol-1-yl)-1,3,4-thiadiazol-2-yl)-2-oxo-2H-pyran-6-carboxamide as “halo-pyrone reagent” and (R)-2-methoxy-1-phenylethan-1-amine as the “amine reagent”. LCMS (ESI, m/z)=527.20 [M+1]+. ¹H NMR (400 MHz, DMSO-d6) δ 13.38 (br, 1H), 7.74 (s, 1H), 7.44-7.40 (m, 4H), 7.31-7.27 (m, 1H), 7.15 (s, 1H), 6.82-6.76 (m, 1H), 6.40 (s, 1H), 5.15-5.06 (m, 1H), 4.14-4.02 (m, 2H), 3.70-3.59 (m, 4H), 3.34-3.24 (s, 6H), 2.65 (s, 3H).

### Example 16: (R)—N-(5-(3-chloro-1-methyl-1H-pyrrol-2-yl)-1,3,4-thiadiazol-2-yl)-4-((2-methoxy-1-phenylethyl)amino)-3-(2-methoxyethoxy)-2-oxo-2H-pyran-6-carboxamide (Compound 201)

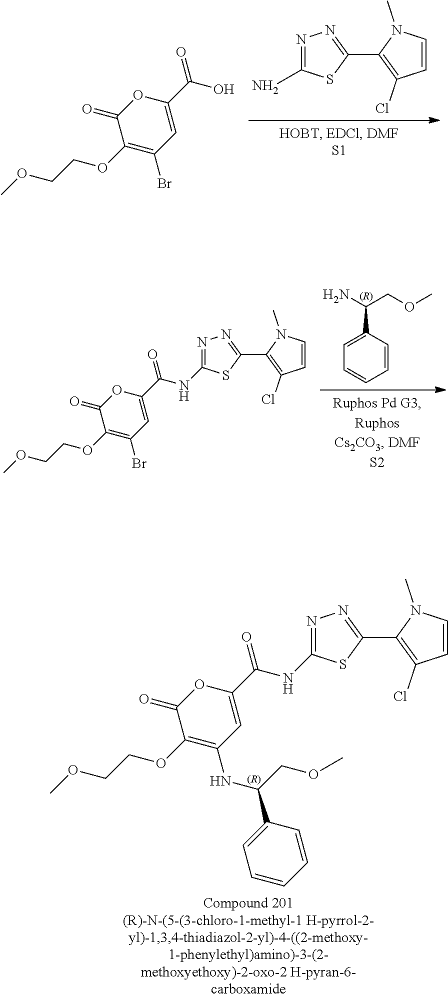

(R)—N-(5-(3-Chloro-1-methyl-1H-pyrrol-2-yl)-1,3,4-thiadiazol-2-yl)-4-((2-methoxy-1-phenylethyl)amino)-3-(2-methoxyethoxy)-2-oxo-2H-pyran-6-carboxamide (Compound 201) was prepared according to Example 15 steps 3-4 using 5-(3-chloro-1-methyl-1H-pyrrol-2-yl)-1,3,4-thiadiazol-2-amine in place of 5-(5-methyl-1H-pyrazol-1-yl)-1,3,4-thiadiazol-2-amine and 4-bromo-5-(2-methoxyethoxy)-6-oxopyran-2-carboxylic acid (product of Example 15, Step 2) to provide 4-bromo-N-(5-(3-chloro-1-methyl-1H-pyrrol-2-yl)-1,3,4-triadiazol-2-yl)-3-(2-methoxyethoxy)-2-oxo-2H-pyran-6-carboxamide as the “halo-pyrone reagent”, followed by coupling with (R)-2-methoxy-1-phenylethan-1-amine as the “amine reagent”. LCMS (ESI, m/z)=560.20 [M+1]+. ¹H NMR (300 MHz, DMSO-d6) δ 13.33 (br, 1H), 7.48-7.30 (m, 5H), 7.31-7.14 (m, 2H), 6.82-6.76 (m, 1H), 6.32 (d, J=2.8 Hz, 1H), 5.15-5.06 (m, 1H), 4.16-4.03 (m, 2H), 3.94 (s, 3H), 3.70-3.58 (m, 4H), 3.33 (s, 6H).

### Example 17: (S)-3-methoxy-4-(3-(methoxymethyl)pyrrolidin-1-yl)-N-(5-(5-methyl-1H-pyrazol-1-yl)-1,3,4-thiadiazol-2-yl)-2-oxo-2H-pyran-6-carboxamide (Compound 87)

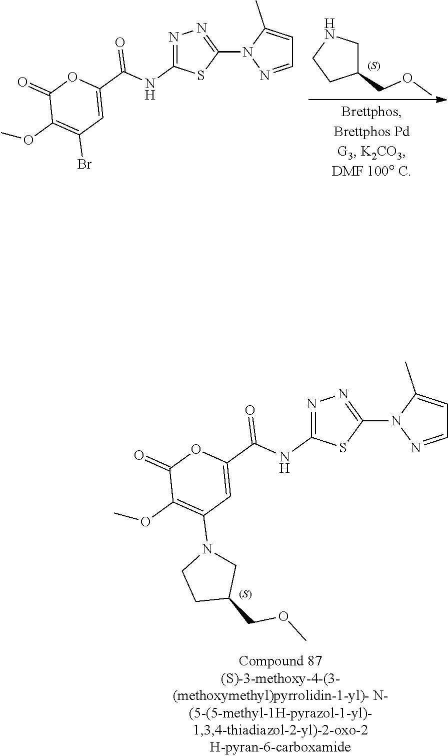

Into a mixture of 4-bromo-5-methoxy-N-[5-(5-methylpyrazol-1-yl)-1,3,4-thiadiazol-2-yl]-6-oxopyran-2-carboxamide (product of Example 1, Part C, Step 1; “halo-pyrone reagent”) (50 mg, 0.12 mmol, 1.0 equiv) and (3S)-3-(methoxymethyl)pyrrolidine (27 mg, 0.24 mmol, 2.0) equiv) (“amine reagent”) in N,N-dimethylformamide (DMF) (0.5 mL) was added 2-(dicyclohexylphosphino)3,6-dimethoxy-2′,4′,6′-triisopropyl-1,1′-biphenyl (BrettPhos) (26 mg, 0.048 mmol, 0.40 equiv), [(2-di-tert-butylphosphino-3,6-dimethoxy-2′,4′,6′-triisopropyl-1,1′-biphenyl)-2-(2′-amino-1,1′-biphenyl)]palladium(11) methanesulfonate (BrettPhos Pd G3) (21 mg, 0.024 mmol, 0.20 equiv) and K₂CO₃ (50 mg, 0.36 mmol, 3.0 equiv). The resulting mixture was stirred for 1 h at 100° C. under N₂ (nitrogen gas). The mixture was then purified by C18 reverse phase flash chromatography (acetonitrile (MeCN) in water (0.1% trifluoroacetic acid (TFA)), 10% to 50% gradient in 10 min; detector, UV 254 nm) to afford (S)-3-methoxy-4-(3-(methoxymethyl)pyrrolidin-1-yl)-N-(5-(5-methyl-1H-pyrazol-yl)-1,3,4-thiadiazol-2-yl)-2-oxo-2H-pyran-6-carboxamide (Compound 87) (20 mg, 38% yield). LCMS (ESI, m/z)=447.1 [M+1]+¹H NMR (400 MHz, DMSO-d6) δ 7.61 (s, 1H), 6.92 (s, 1H), 6.30 (s, 1H), 3.79-3.64 (m, 2H), 3.61 (s, 4H), 3.42-3.38 (m, 3H), 3.38-3.35 (m, 1H), 3.29 (s, 3H), 2.60 (s, 3H), 2.02-1.98 (m, 1H), 1.68-1.66 (m, 1H).

### Example 18: (R)—N-(5-(3-chlorothiophen-2-yl)-1,3,4-thiadiazol-2-yl)-3-methoxy-4-((1-methoxypropan-2-yl)amino)-2-oxo-2H-pyran-6-carboxamide (Compound 94)

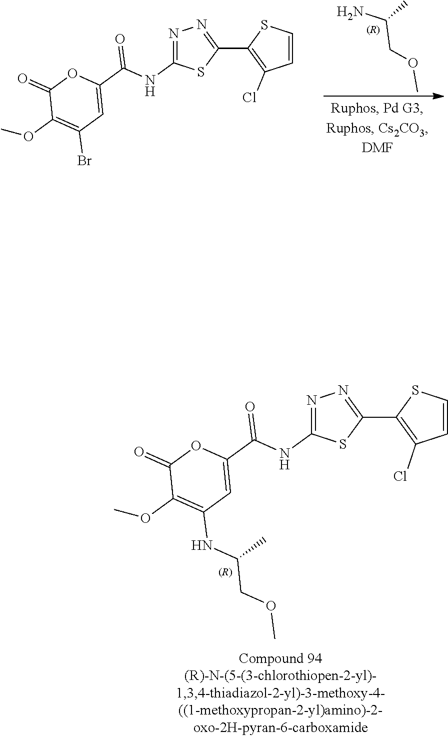

(R)—N-(5-(3-Chlorothiophen-2-yl)-1,3,4-thiadiazol-2-yl)-3-methoxy-4-((1-methoxypropan-2-yl)amino)-2-oxo-2H-pyran-6-carboxamide (Compound 94) was prepared according to Example 1, Part C, Step 2 using 4-bromo-N-(5-(3-chlorothiophen-2-yl)-1,3,4-thiadiazol-2-yl)-3-methoxy-2-oxo-2H-pyran-6-carboxamide (product of Example 5, Step 2) as “halo-pyrone reagent” and (R)-1-methoxypropan-2-amine as the “amine reagent”. LCMS (ESI, m/z)=457.00 [M+1]+. ¹H NMR (400 MHz, DMSO-d6) δ 7.68 (d, J=5.2 Hz, 1H), 7.16 (d, J=5.2 Hz, 1H), 7.06 (s, 1H), 6.38-6.36 (m, 11H), 3.91-3.85 (m, 1H), 3.65 (s, 3H), 3.41-3.38 (m, 2H), 3.29 (s, 3H), 3.29 (s, 3H), 1.18 (d, J=6.4 Hz, 1H, 3H).

### Example 19: (S)-3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide (Compound 100*) and (R)-3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide (Compound 101*)

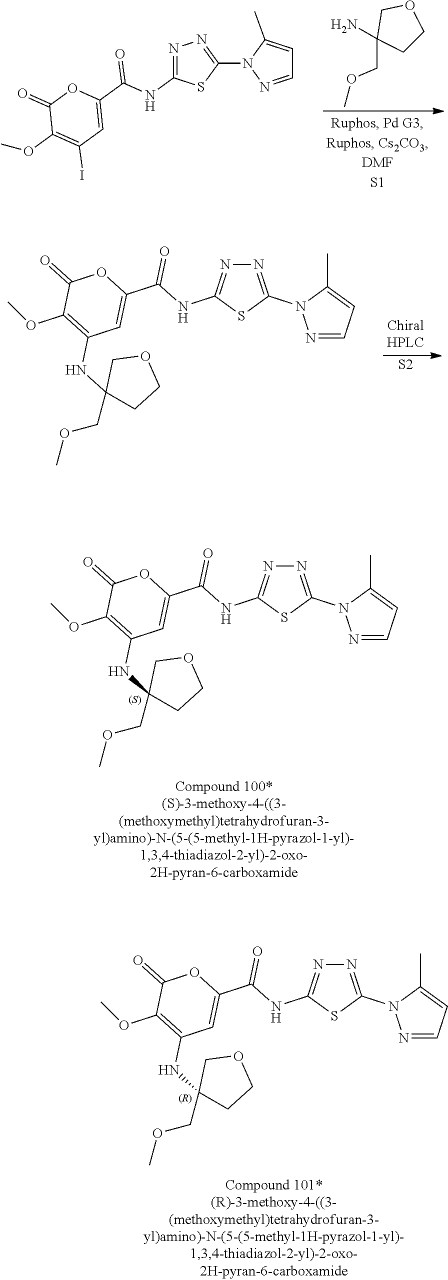

Racemic 3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and 3-(methoxymethyl)tetrahydrofuran-3-amine as the “amine reagent”. Separation of constituent enantiomers of racemic 3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide by Prep-Chiral-HPLC (Chiral ART Cellulose-SA, 2*25 cm, 5 μm. Mobile Phase A: Hexanes (0.1% trifluoroacetic acid (TFA)). Mobile Phase B: ethanol (EtOH):dichloromethane (DCM)=1:1; Flow rate: 20 mL/min; Gradient: 50% B to 50% B in 9.5 min; Wave Length: 220/254 nm; RT1 (min): 7.15; RT2 (min): 9.27; Sample Solvent: methanol (MeOH):DCM=1:1) provided two enantiomers with arbitrarily assigned stereochemistry: (S)-3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide (Compound 100*), first eluting peak. LCMS (ESI, m/z)=463.1 [M+1]+, 1H NMR (400 MHz, DMSO-d6) δ 7.79 (s, 1H), 7.21 (s, 1H), 6.63 (s, 1H), 6.45 (s, 1H), 3.99-3.78 (m, 4H), 3.72 (s, 3H), 3.61 (d, J=9.6 Hz, 1H), 3.53 (d, J=9.6 Hz, 1H), 3.33 (s, 3H), 2.68 (s, 3H), 2.22-2.18 (m, 2H); and (R)-3-methoxy-4-((3-(methoxymethyl)tetrahydrofuran-3-yl)amino)-N-(5-(5-methyl-1H-pyrazol-1-yl)-1,3,4-thiadiazol-2-yl)-2-oxo-2H-pyran-6-carboxamide (Compound 101*), second eluting peak. LCMS (ESI, m/z)=463.1 [M+1]+, ¹H NMR (400 MHz, DMSO-d6) δ 7.79 (s, 1H), 7.21 (s, 1H), 6.63 (s, 1H), 6.45 (s, 1H), 3.99-3.78 (m, 4H), 3.72 (s, 3H), 3.61 (d, J=9.6 Hz, 1H), 3.53 (d, J=9.6 Hz, 1H), 3.33 (s, 3H), 2.68 (s, 3H), 2.22-2.18 (m, 2H).

### Example 20: 3-methoxy-4-(((cis)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 108, Rac-108), 3-methoxy-4-(((trans)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 109, Rac-109), 3-methoxy-4-(((1S,3R)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 108a*) and 3-methoxy-4-(((1R,3S)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 108b*) and 3-methoxy-4-(((1S,3S)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 109a*) and 3-methoxy-4-(((1R,3R)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 109b*)

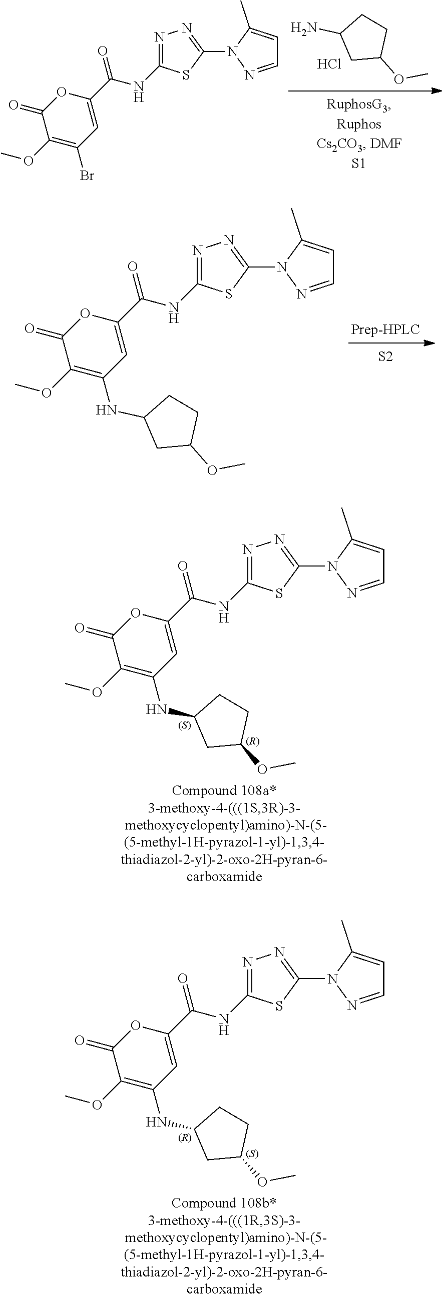

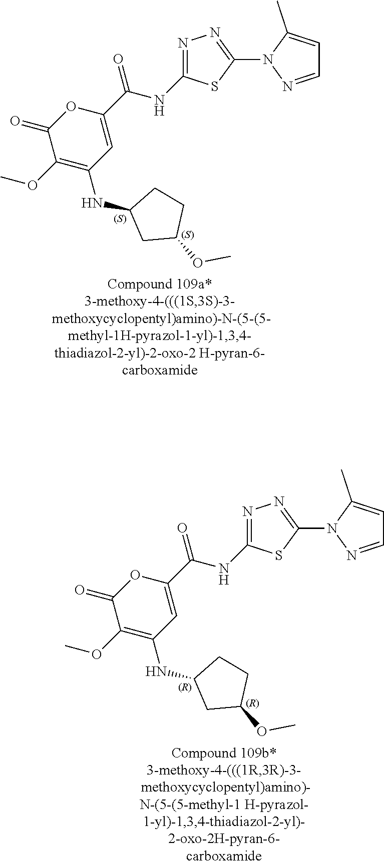

3-Methoxy-4-((3-methoxycyclopentyl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using 3-methoxycyclopentan-1-amine hydrochloride as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent”. Separation of diastereoisomers using Prep-HPLC (XBridge Prep Phenyl OBD Column, 19*150 mm, 5 μm; Mobile Phase A: Water (10 mmol/L NH4HCO3). Mobile Phase B: acetonitrile (MeCN); Flow rate: 60 mL/min; Gradient: 15% B to 20% B in 8 min. 20% B; Wave Length: 254 nm) provided two diastereomeric sets of enantiomers with arbitrarily assigned stereochemistry: 3-methoxy-4-(((1S,3R)-3-methoxycyclopentyl)amino)-N-(5-(5-methyl-H-pyrazol-yl)-1,3,4-thiadiazol-2-yl)-2-oxo-2H-pyran-6-carboxamide (Compound 108a*) and 3-methoxy-4-(((1R,3S)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 108b*) isolated as a racemic mixture. LCMS (ESI, m/z)=447.15 [M+1]+. ¹H NMR (400 MHz, DMSO-d6) δ 7.66 (d, J=1.6 Hz, 1H), 7.11 (s, 1H), 6.70 (d, J=8.0 Hz, 1H), 6.35 (d, J=1.6 Hz, 1H), 4.15-4.10 (m, 1H), 3.91-3.87 (m, 1H), 3.65 (s, 3H), 3.21 (s, 3H), 2.62 (s, 3H), 2.12-1.90 (m, 3H), 1.83-1.78 (m, 1H), 1.71-1.50 (m, 2H); and 3-methoxy-4-(((1S,3S)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 109a*) and 3-methoxy-4-(((1R,3R)-3-methoxycyclopentyl)amino)-N-(5-(5-methyl-1H-pyrazol-1-yl)-1,3,4-thiadiazol-2-yl)-2-oxo-2H-pyran-6-carboxamide (Compound 109b*) isolated as a racemic mixture. LCMS (ESI, m/z)=447.20 [M+1]+. ¹H NMR (400 MHz, DMSO-d6) δ 7.68 (d, J=1.6 Hz, 1H), 7.15 (s, 1H), 6.52 (br, 1H), 6.36 (d, J=1.6 Hz, 1H), 4.09-4.04 (m, 1H), 3.86-3.82 (m, 1H), 3.66 (s, 3H), 3.22 (s, 3H), 2.63 (s, 3H), 2.22-2.15 (m, 1H), 1.97-1.91 (m, 1H), 1.85-1.64 (m, 4H).

### Example 21: 4-((3-hydroxybicyclo[1.1.1]pentan-1-yl)amino)-3-methoxy-N-(5-(5-methyl-1H-pyrazol-1-yl)-1,3,4-thiadiazol-2-yl)-2-oxo-2H-pyran-6-carboxamide (Compound 126) and 3-methoxy-4-((3-methoxybicyclo[1.1.1]pentan-1-yl)amino)-N-(5-(5-methyl-1H-pyrazol-1-yl)-1,3,4-thiadiazol-2-yl)-2-oxo-2H-pyran-6-carboxamide (Compound 126-OMe)

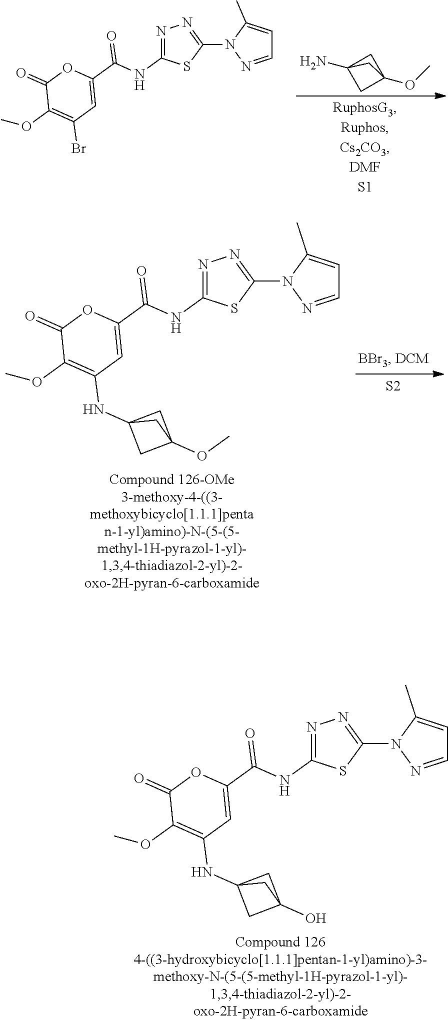

Step 1: 5-methoxy-4-({3-methoxybicyclo[1.1.1]pentan-1-yl}amino)-N-[5-(5-methylpyrazol-1-yl)-1,3,4-thiadiazol-2-yl]-6-oxopyran-2-carboxamide (Compound 126-OMe) was prepared according to Example 1, Part C, Step 2 using 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent” and 3-methoxybicyclo[1.1.1]pentan-1-amine as the “amine reagent”. LCMS (ESI, m/z)=445.1 [M+1]+. ¹H NMR (400 MHz, DMSO-d6) δ 7.67 (d, J=1.8 Hz, 1H), 7.21 (s, 1H), 6.36 (s, 1H), 3.64 (d, J=2.4 Hz, 3H), 3.24 (d, J=2.3 Hz, 3H), 2.60 (s, 3H), 2.24 (d, J=2.3 Hz, 6H).

Step 2: Into a solution of 5-methoxy-4-({3-methoxybicyclo[1.1.1]pentan-1-yl}amino)-N-[5-(5-methylpyrazol-1-yl)-1,3,4-thiadiazol-2-yl]-6-oxopyran-2-carboxamide (Compound 126-OMe) (80 mg, 0.180 mmol, 1.00 equiv) in dichloromethane (DCM) (3 mL) was added a solution of BBr₃ (0.80 mL, 0.80 mmol, 4.44 equiv) in DCM (1 M) at 0° C. The resulting mixture was stirred for 30 min at 0° C. The reaction was quenched with methanol (MeOH) at 0° C., and then directly purified by C18 reverse phase flash chromatography (acetonitrile (MeCN) in water, 10% to 50% gradient in 10 min; detector, UV 254 nm) to afford 4-((3-hydroxybicyclo[1.1.1]pentan-1-yl)amino)-5-methoxy-N-[5-(5-methylpyrazol-1-yl)-1,3,4-thiadiazol-2-yl]-6-oxopyran-2-carboxamide (Compound 126) (12 mg, 15% yield). LCMS (ESI, m/z)=431.10 [M+1]+. ¹H NMR (400 MHz, DMSO-d6) δ 13.50 (s, 1H), 7.79 (d, J=1.6 Hz, 1H), 7.68 (s, 1H), 7.24 (s, 1H), 6.45 (d, J=1.6 Hz, 1H), 6.38 (s, 1H), 3.69 (s, 3H), 2.68 (s, 3H), 2.21 (s, 6H).

### Example 22: N-(5-(3-chloro-1-methyl-1H-pyrrol-2-yl)-1,3,4-thiadiazol-2-yl)-4-((2-hydroxyethyl)amino)-3-methoxy-2-oxo-2H-pyran-6-carboxamide (Compound 129) and N-(5-(3-chloro-1-methyl-1H-pyrrol-2-yl)-1,3,4-thiadiazol-2-yl)-3-methoxy-4-((2-methoxyethyl)amino)-2-oxo-2H-pyran-6-carboxamide (Compound 129-OMe)

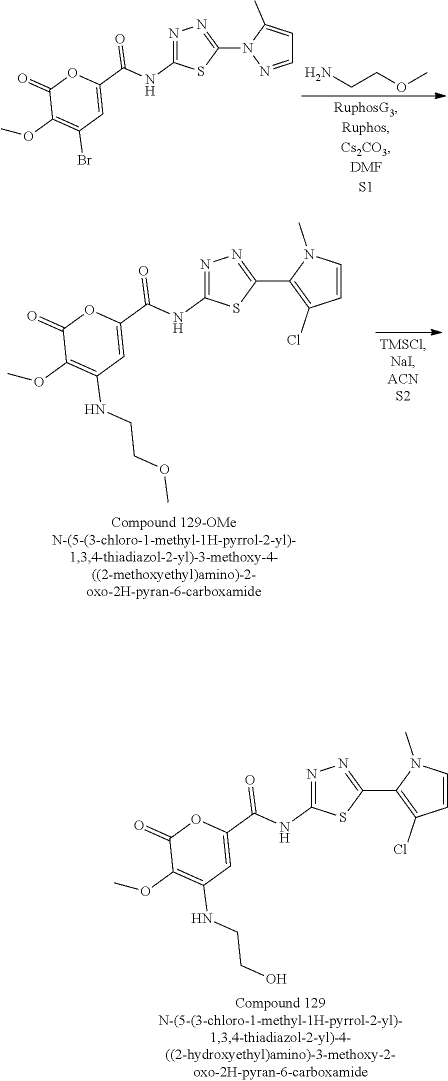

Step 1: N-[5-(3-chloro-1-methylpyrrol-2-yl)-1,3,4-thiadiazol-2-yl]-5-methoxy-4-[(2-methoxyethyl)amino]-6-oxopyran-2-carboxamide (Compound 129-OMe) was prepared according to Example 10, Step 7 using 4-bromo-N-(5-(3-chloro-1-methyl-1H-pyrrol-2-yl)-1,3,4-thiadiazol-2-yl)-3-methoxy-2-oxo-2H-pyran-6-carboxamide (product of Example 10, Step 6) as the “halo-pyrone reagent” and 2-methoxyethan-1-amine as the “amine reagent”. LCMS (ESI, m/z)=440.0 [M+1]+. ¹H NMR (400 MHz, DMSO-d6) δ 13.36 (s, 1H), 7.32 (s, 1H), 7.13 (d, J=3.0 Hz, 1H), 7.01 (s, 1H), 6.31 (d, J=2.9 Hz, 1H), 3.94 (s, 3H), 3.68 (s, 3H), 3.48 (s, 2H), 2.48 (s, 1H).

Step 2: Into a solution of N-[5-(3-chloro-1-methylpyrrol-2-yl)-1,3,4-thiadiazol-2-yl]-5-methoxy-4-[(2-methoxyethyl)amino]-6-oxopyran-2-carboxamide (Compound 129-OMe) (180 mg, 0.41 mmol, 1.00 equiv) in acetonitrile (MeCN) (3 mL) were added NaI (307 mg, 2.04 mmol, 5.00 equiv) and trimethylsilyl chloride (TMSCl) (222 mg, 2.04 mmol, 5.00 equiv) at room temperature. The resulting mixture was stirred overnight at 80° C. then purified directly by C18 reverse phase flash chromatography (acetonitrile (MeCN) in water, 10% to 50% gradient in 10 min; detector, UV 254 nm) to afford a crude product (40 mg) which was further purified by Prep-HPLC (YMC-Actus Triart C18 ExRS, 30*150 mm, 5 μm; mobile phase, water (10 mmol/L NH₄HCO₃) and MeCN (15% MeCN up to 25% in 10 min); Detector, UV 254 nm) to afford N-(5-(3-chloro-1-methyl-1H-pyrrol-2-yl)-1,3,4-thiadiazol-2-yl)-4-((2-hydroxyethyl)amino)-3-methoxy-2-oxo-2H-pyran-6-carboxamide (Compound 129) (4.3 mg, 2.5% yield). LCMS (ESI, m/z)=426.00 [M+1]+. ¹H NMR (400 MHz, DMSO-d6) δ 7.26 (s, 1H), 7.09 (d, J=2.8 Hz, 1H), 6.89 (s, 1H), 6.28 (d, J=2.8 Hz, 1H), 4.87 (t, J=5.6 Hz, 1H), 3.92 (s, 3H), 3.68 (s, 3H), 3.56-3.49 (m, 2H), 3.38-3.35 (m, 2H).

### Example 23: 4-((2-hydroxyethyl)amino)-3-methoxy-N-(5-(5-methyl-1H-pyrazol-1-yl)-1,3,4-thiadiazol-2-yl)-2-oxo-2H-pyran-6-carboxamide (Compound 130)

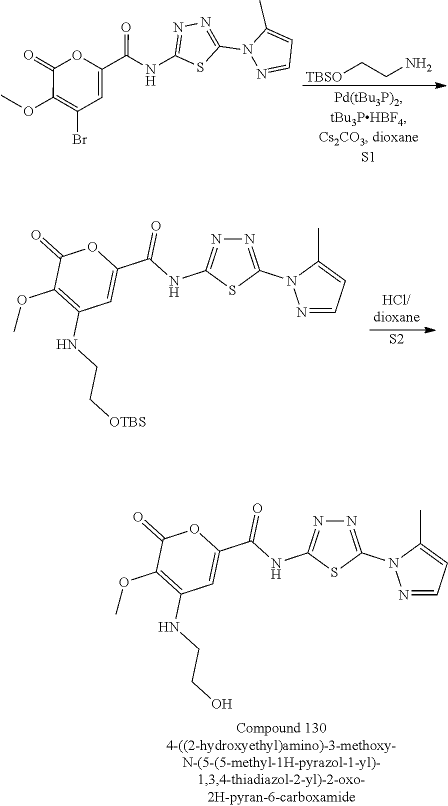 [SMILES: COc1c(Br)cc(C(=O)Nc2nnc(-n3nccc3C)s2)oc1=O ; CC(C)(C)[Si](C)(C)OCCN ; COc1c(NCCO[Si](C)(C)C(C)(C)C)cc(C(=O)Nc2nnc(-n3nccc3C)s2)oc1=O ; "S2" ; COc1c(NCCO)cc(C(=O)Nc2nnc(-n3nccc3C)s2)oc1=O]

Step 1: To a stirred mixture of 4-bromo-5-methoxy-N-[5-(5-methylpyrazol-1-yl)-1,3,4-thiadiazol-2-yl]-6-oxopyran-2-carboxamide (product of Example 1, Part C, Step 1; “halo-pyrone reagent”) (200 mg, 0.485 mmol, 1.00 equiv) and (2-aminoethoxy)(tert-butyl)dimethylsilane (“amine reagent”) (851 mg, 4.85 mmol, 10.0 equiv) in dioxane (5 mL) was added bis(tri-tert-butylphosphine)palladium(0) (Pd(t-Bu₃P)₂) (37 mg, 0.073 mmol, 0.15 equiv), tri-tert-butylphosphonium tetrafluoroborate (t-Bu₃P—HBF₄) (21 mg, 0.073 mmol, 0.15 equiv) and cesium carbonate (316 mg, 0.97 mmol, 2.0 equiv) at room temperature. The resulting mixture was stirred for 4 h at 120° C. under N₂ (nitrogen gas). The mixture was then poured into water and extracted with ethyl acetate (EtOAc) (3×10 mL). The combined organic layers were washed with water and brine (3×10 mL), and dried over anhydrous Na₂SO₄. After filtration, the filtrate was concentrated under reduced pressure and the residue was purified by C18 reverse phase flash chromatography (acetonitrile (MeCN) in water (10 mmol/L NH₄HCO₃), 10% to 50% gradient in 10 min; detector, UV 254 nm) to afford 4-({2-[(tert-butyldimethylsilyl)oxy]ethyl}amino)-5-methoxy-N-[5-(5-methylpyrazol-1-yl)-1,3,4-thiadiazol-2-yl]-6-oxopyran-2-carboxamide (48 mg, 19% yield). LCMS (ESI, m/z)=507.2 [M+1]+. TBS=tertbutyldimethylsilyl.

Step 2: A solution of 4-({2-[(tert-butyldimethylsilyl)oxy]ethyl}amino)-5-methoxy-N-[5-(5-methylpyrazol-1-yl)-1,3,4-thiadiazol-2-yl]-6-oxopyran-2-carboxamide (30 mg, 0.059 mmol, 1.00 equiv) in HCl (4 M in 1,4-dioxane) (1.5 mL) was stirred for 1 h at room temperature. The mixture was then concentrated under reduced pressure and purified by Prep-HPLC (conditions (SHIMADZU: Column, XBridge Shield RP18 OBD Column, 30*150 mm, 5 μm; mobile phase, Water (10 mmol/L NH₄HCO₃) and acetonitrile (MeCN) (8% MeCN up to 20% in 10 min); Detector, UV 254 nm) to afford 4-((2-hydroxyethyl)amino)-3-methoxy-N-(5-(5-methyl-1H-pyrazol-1-yl)-1,3,4-thiadiazol-2-yl)-2-oxo-2H-pyran-6-carboxamide (Compound 130) (9.3 mg, 40% yield). LCMS (ESI, m/z)=393.0 [M+1]+. ¹H NMR (400 MHz, DMSO-d6) δ 7.70 (d, J=1.6 Hz, 1H), 7.22 (s, 1H), 6.85 (t, J=6.4 Hz, 1H), 6.38 (d, J=1.6 Hz, 1H), 4.88 (t, J=5.6 Hz, 1H), 3.68 (s, 3H), 3.56-3.53 (m, 2H), 3.37-3.34 (m, 2H), 2.64 (s, 3H).

### Example 24: N-(5-(3-chloro-1-methyl-1H-pyrrol-2-yl)-1,3,4-thiadiazol-2-yl)-3-methoxy-4-(((1R,2S)-2-methoxycyclobutyl)amino)-2-oxo-2H-pyran-6-carboxamide (Compound 132*) and N-(5-(3-chloro-1-methyl-1H-pyrrol-2-yl)-1,3,4-thiadiazol-2-yl)-3-methoxy-4-(((1S,2R)-2-methoxycyclobutyl)amino)-2-oxo-2H-pyran-6-carboxamide (Compound 133*)

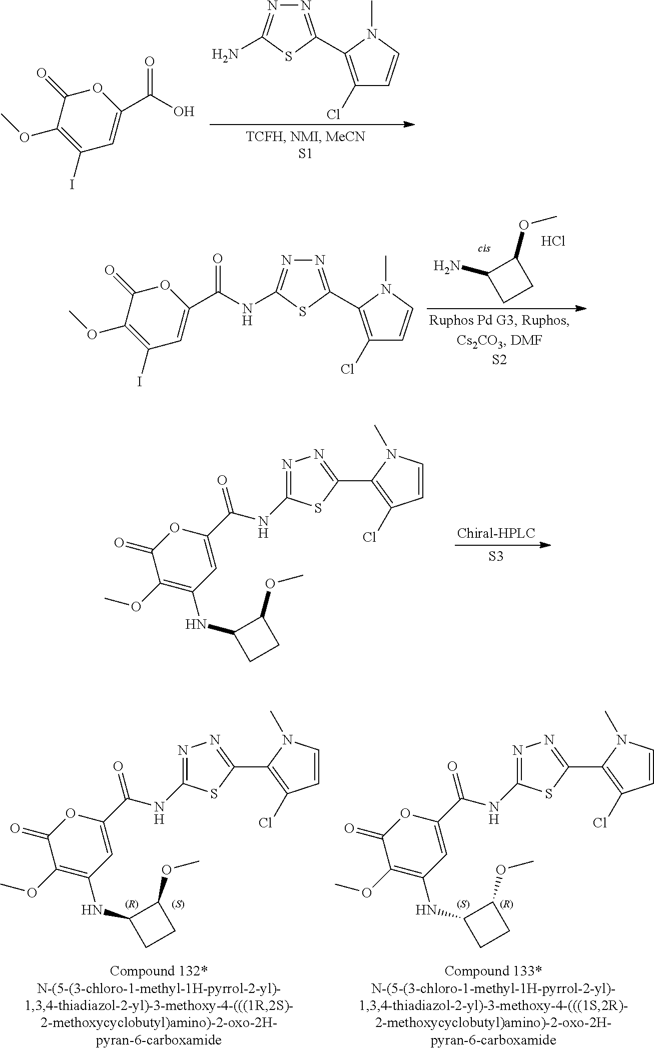

Step 1: Into a solution of 4-iodo-5-methoxy-6-oxopyran-2-carboxylic acid (product of Example 6, Step 3) (150 mg, 0.51 mmol, 1.00 equiv) in acetonitrile (MeCN) (2 mL) was added chloro-N,N,N′,N′-tetramethylformamidinium hexafluorophosphate (TCFH) (200 mg, 0.713 mmol, 1.41 equiv) and N-methylimidazole (NMI) (166 mg, 2.02 mmol, 3.99 equiv) at room temperature. To the mixture was added 5-(3-chloro-1-methylpyrrol-2-yl)-1,3,4-thiadiazol-2-amine (product of Example 10, step 5) (109 mg, 0.51 mmol, 1 equiv) and the resulting mixture was stirred for 0.5 h at room temperature then concentrated under reduced pressure. The resulting residue was purified by C18 reverse phase flash chromatography (eluted with acetonitrile/water (5:1)) to afford N-[5-(3-chloro-1-methylpyrrol-2-yl)-1,3,4-thiadiazol-2-yl]-4-iodo-5-methoxy-6-oxopyran-2-carboxamide (120 mg, 47% yield).

Step 2: Racemic N-(5-(3-chloro-1-methyl-1H-pyrrol-2-yl)-1,3,4-thiadiazol-2-yl)-3-methoxy-4-(((1,2-cis)-2-methoxycyclobutyl)amino)-2-oxo-2H-pyran-6-carboxamide was prepared and its constituent enantiomers separated according to Example 14 using N-[5-(3-chloro-1-methylpyrrol-2-yl)-1,3,4-thiadiazol-2-yl]-4-iodo-5-methoxy-6-oxopyran-2-carboxamide as the “halo-pyrone reagent” and rac-(1,2-cis)-2-methoxycyclobutan-1-amine hydrochloride as the “amine reagent” to provide two enantiomers with arbitrarily assigned stereochemistry: N-(5-(3-chloro-1-methyl-1H-pyrrol-2-yl)-1,3,4-thiadiazol-2-yl)-3-methoxy-4-(((1R,2S)-2-methoxycyclobutyl)amino)-2-oxo-2H-pyran-6-carboxamide (Compound 132*), first eluting peak. LCMS (ESI, m/z)=466.05 [M+1]+, ¹H NMR (4001 MHz, DMSO-d6) δ 7.20 (s, 1H), 7.12 (d, J=2.8 Hz, 1H), 6.58 (br, 1H), 6.31 (d, J=2.8 Hz, 1H), 4.41-4.35 (m, 1H), 4.10-4.06 (m, 1H), 3.93 (s, 3H), 3.72 (s, 3H), 3.22 (s, 3H), 2.19-1.97 (m, 4H); and N-(5-(3-chloro-1-methyl-1H-pyrrol-2-yl)-1,3,4-thiadiazol-2-yl)-3-methoxy-4-(((1S,2R)-2-methoxycyclobutyl)amino)-2-oxo-2H-pyran-6-carboxamide (Compound 133*), second eluting peak, LCMS (ESI, m/z)=466.05 [M+1]+, ¹H NMR (400 MHz, DMSO-d6) δ 7.20 (s, 1H), 7.12 (d, J=2.8 Hz, 1H), 6.58 (br, 1H), 6.31 (d, J=2.8 Hz, 1H), 4.41-4.35 (m, 1H), 4.10-4.06 (n, 1H), 3.93 (s, 3H), 3.72 (s, 3H), 3.22 (s, 3H), 2.19-1.97 (m, 4H).

### Example 25: N-(5-(5-chloro-4-fluoro-1H-pyrazol-1-yl)-1,3,4-thiadiazol-2-yl)-5-methoxy-4-(2-methoxyethylamino)-6-oxo-6H-pyran-2-carboxamide (Compound 134)

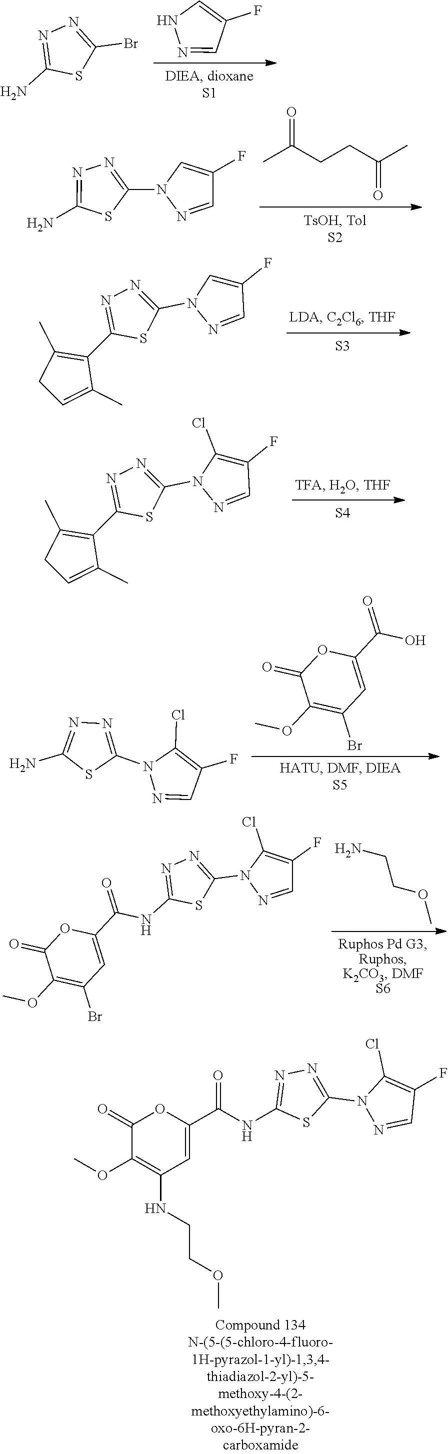

Step 1: To a stirred solution of 5-bromo-1,3,4-thiadiazol-2-amine (13.8 g, 76.6 mmol, 1.00 equiv) and 4-fluoro-1H-pyrazole (7.96 g, 92.5 mmol, 1.21 equiv) in dioxane (80 mL) was added diisopropylethylamine (DIEA) (29.8 g, 231 mmol, 3.01 equiv.) at room temperature. The resulting mixture was stirred overnight at 80° C. The mixture was then diluted with water and extracted with ethyl acetate (EtOAc) (3×1 L). The combined organic layers were washed with water (3×100 mL) and dried over anhydrous Na₂SO₄. After filtration, the filtrate was concentrated under reduced pressure. The residue was diluted in EtOAc (50 mL) and the precipitated solids were collected by filtration and washed with EtOAc (3×10 mL) to provide 5-(4-fluoropyrazol-1-yl)-1,3,4-thiadiazol-2-amine (5.0 g, 35% yield). LCMS (ESI, m/z)=186 [M+1]+.

Step 2: To a stirred solution of 5-(4-fluoropyrazol-1-yl)-1,3,4-thiadiazol-2-amine (5.0 g, 27 mmol, 1.0 equiv) and 2,5-hexanedione (4.62 g, 40.5 mmol, 1.50 equiv) in toluene (20 mL) was added tosic acid (TsOH) (0.93 g, 5.40 mmol, 0.20 equiv) at room temperature. The resulting mixture was stirred for 1 h at 100° C. then concentrated under reduced pressure. The residue was then purified by silica gel column chromatography, eluted with petroleum ether/ethyl acetate (PE/EtOAc) (7:1), to afford 2-(2,5-dimethylpyrrol-1-yl)-5-(4-fluoropyrazol-1-yl)-1,3,4-thiadiazole (3.0 g, 42% yield). LCMS (ESI, m/z): 264 [M+1]+.

Step 3: To a stirred solution of 2-(2,5-dimethylpyrrol-1-yl)-5-(4-fluoropyrazol-1-yl)-1,3,4-thiadiazole (1.0 g, 3.8 mmol, 1.0 equiv) in tetrahydrofuran (THF) (5 mL) was added lithium diisopropyl amide (LDA) (3.80 mL, 3.80 mmol, 1.00 equiv) at −78° C. under N₂ (nitrogen gas). After 30 min, into the above mixture was added hexachloroethane (900 mg, 3.80 mmol, 1.00 equiv) in THF (2 ml) at −78° C. under N₂. The resulting mixture was stirred for 2 h at −78° C. under N₂. The mixture was then quenched with sat. NH₄Cl and extracted with ethyl acetate (EtOAc) (3×100 mL). The combined organic layers were washed with water (3×10 mL), and dried over anhydrous Na₂SO₄. After filtration, the filtrate was concentrated under reduced pressure and the residue was purified by C18 reverse phase flash chromatography (acetonitrile (MeCN) in water (0.1% trifluoroacetic acid (TFA)), 10% to 50% gradient in 10 min; detector, UV 254 nm) to provide 2-(5-chloro-4-fluoropyrazol-1-yl)-5-(2,5-dimethylpyrrol-1-yl)-1,3,4-thiadiazole (600 mg, 53% yield). LCMS (ESI, m/z)=298 [M+1]+.

Step 4: To a stirred solution of 2-(5-chloro-4-fluoropyrazol-1-yl)-5-(2,5-dimethylpyrrol-1-yl)-1,3,4-thiadiazole (200 mg, 0.672 mmol, 1.00 equiv) in H₂O/THF (2:1, 3 mL) was added trifluoroacetic acid (TFA) (2 mL) dropwise at room temperature. The resulting mixture was stirred for 2 h at 50° C. then concentrated under vacuum. The resulting residue was purified by C18 reverse phase flash chromatography (acetonitrile (MeCN) in water, 0% to 100% gradient in 10 min; detector, UV 254 nm) to provide 5-(5-chloro-4-fluoropyrazol-1-yl)-1,3,4-thiadiazol-2-amine (70 mg, 47% yield). LCMS (ESI, m/z)=220 [M+1]+. ¹H NMR (400 MHz, DMSO-d₆) δ 8.16 (d, J=4.3 Hz, 1H), 7.55 (s, 2H).

Step 5: To a stirred solution of 4-bromo-5-methoxy-6-oxopyran-2-carboxylic acid (product of Example 1, Part A, Step 4) (113 mg, 0.45 mmol, 1.00 equiv) and 5-(5-chloro-4-fluoro-1H-pyrazol-1-yl)-1,3,4-thiadiazol-2-amine (100 mg, 0.45 mmol, 1.00 equiv) in N,N-dimethylformamide (DMF) (10 mL) was added 1-((dimethylamino)(dimethyliminio)methyl)-1H-[1,2,3]triazolo[4,5-b]pyridine 3-oxide hexafluorophosphate(V) (HATU) (258 mg, 0.68 mmol, 1.50 equiv) and diisopropylethylamine (DIEA) (176 mg, 1.36 mmol, 3.00 equiv) at 0° C. under N₂ (nitrogen gas). The resulting mixture was stirred at room temperature for 16 h under N₂. The mixture was then diluted with water and extracted with ethyl acetate (EtOAc) (3×50 mL), and the combined organic layers were dried over anhydrous Na₂SO₄. After filtration, the filtrate was concentrated under reduced pressure and the resulting residue was purified by silica gel column chromatography, eluted with methanol/dichloromethane (MeOH/DCM) (1:4), to afford 4-bromo-N-(5-(5-chloro-4-fluoro-1H-pyrazol-1-yl)-1,3,4-thiadiazol-2-yl)-3-methoxy-2-oxo-2H-pyran-6-carboxamide (80 mg, 29% yield). LCMS (EST, m/z)=450.1 [M+1]+.

Step 6: N-(5-(5-Chloro-4-fluoro-1H-pyrazol-1-yl)-1,3,4-thiadiazol-2-yl)-5-methoxy-4-(2-methoxyethylamino)-6-oxo-6H-pyran-2-carboxamide (Compound 134) was prepared according to Example 1, Part C, Step 2 using 4-bromo-N-(5-(5-chloro-4-fluoro-1H-pyrazol-1-yl)-1,3,4-thiadiazol-2-yl)-3-methoxy-2-oxo-2H-pyran-6-carboxamide as the “halo-pyrone reagent” and 2-methoxyethan-1-amine as the “amine reagent”. LCMS (ESI, m/z)=445.0 [M+1]+. 1H NMR (400 MHz, CD₃OD) δ 7.97 (d, J=4.4 Hz, 1H), 7.42 (s, 1H), 3.83 (s, 3H), 3.58 (s, 3H), 3.39 (s, 4H).

### Example 26: N-(5-(2-chlorothiophen-3-yl)-1,3,4-thiadiazol-2-yl)-3-methoxy-4-((2-methoxyethyl)amino)-2-oxo-2H-pyran-6-carboxamide (Compound 135)

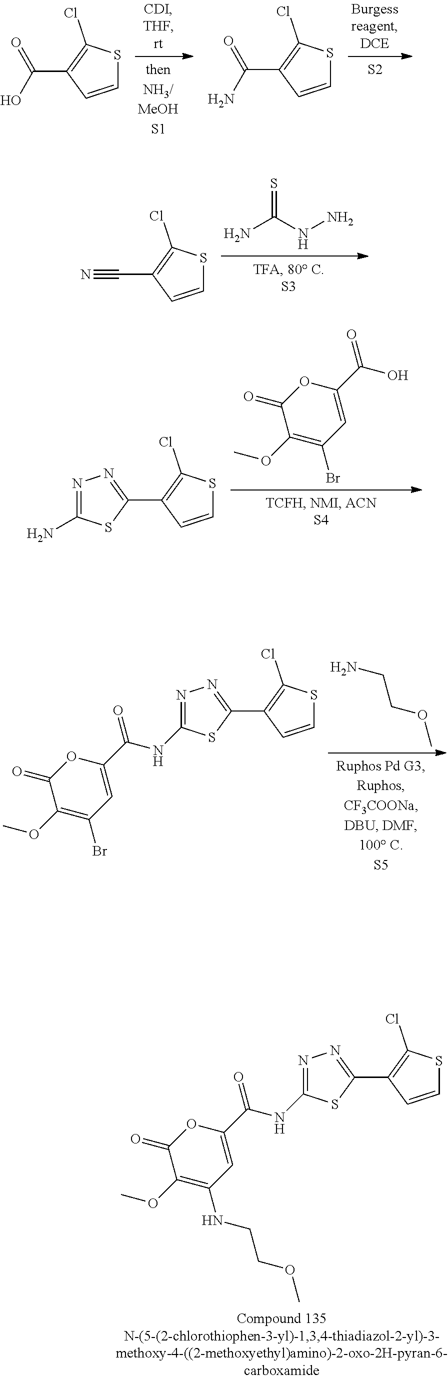

Step 1: To a stirred solution of 2-chlorothiophene-3-carboxylic acid (1.0 g, 6.15 mmol, 1.0 equiv) in tetrahydrofuran (THF) (10 mL) was added carbonyldiimidazole (CDI) (1.50 g, 9.22 mmol, 1.5 equiv) in portions at room temperature. The resulting mixture was stirred for 1 h at room temperature. To the above mixture was added an ammonia in methanol solution (7N NH₃/MeOH) (10 mL) dropwise over 1 h at room temperature. The resulting mixture was stirred for an additional 30 min at room temperature then concentrated under reduced pressure. The residue was then purified by C18 reverse phase flash chromatography (acetonitrile (MeCN) in water (0.1% trifluoroacetic acid (TFA)), 10% to 50% gradient in 10 min) to provide 2-chlorothiophene-3-carboxamide (770 mg, 77% yield). LCMS (ESI, m/z)=162 [M+1]⁺.

Step 2: To a stirred solution of 2-chlorothiophene-3-carboxamide (350 mg, 2.17 mmol, 1.00 equiv) in dichloroethane (DCE) (5 mL) was added methyl N-(triethylammoniumsulfonyl)carbamate (Burgess reagent) (1.50 g, 6.50 mmol, 3.00 equiv) at room temperature. The resulting mixture was stirred for 1 h at 60° C. then concentrated under reduced pressure. The resulting residue was then purified by C18 reverse phase flash chromatography (MeCN in water (0.1% trifluoroacetic acid (TFA)), 10% to 50% gradient in 10 min) to provide 2-chlorothiophene-3-carbonitrile (200 mg, 64% yield). LCMS (ESI, m/z)=144 [M+1]+.

Step 3: To a stirred solution of 2-chlorothiophene-3-carbonitrile (1.20 g, 8.36 mmol, 1.00 equiv) in trifluoroacetic acid (TFA) (10 mL) was added thiosemicarbazide (1.14 g, 12.5 mmol, 1.50 equiv) at room temperature. The resulting mixture was stirred for 1 h at 80° C. then concentrated under reduced pressure. The residue was then purified by C18 reverse phase flash chromatography (MeCN in water, 30% to 40% gradient in 10 min) to provide 5-(2-chlorothiophen-3-yl)-1,3,4-thiadiazol-2-amine (800 mg, 44% yield). LCMS (ESI, m/z)=218 [M+1]+.

Step 4: To a stirred solution of 5-(2-chlorothiophen-3-yl)-1,3,4-thiadiazol-2-amine (100 mg, 0.46 mmol, 1.0 equiv) in acetonitrile (3 mL) was added chloro-N,N,N′,N′-tetramethylformamidinium hexafluorophosphate (TCFH) (385 mg, 1.37 mmol, 2.99 equiv), N-methylimidazole (NMI) (376 mg, 4.58 mmol, 9.97 equiv) and 4-bromo-5-methoxy-6-oxopyran-2-carboxylic acid (product of Example 1, Part A, Step 4) (137 mg, 0.55 mmol, 1.20 equiv) at room temperature. The resulting mixture was stirred for 1 h at room temperature. The resulting solids were collected by filtration and washed with acetonitrile (5×4 mL). The collected solids were then purified by C18 reverse phase flash chromatography (MeCN in water, 10% to 50% gradient in 10 min) to provide 4-bromo-N-[5-(2-chlorothiophen-3-yl)-1,3,4-thiadiazol-2-yl]-5-methoxy-6-oxopyran-2-carboxamide (70 mg, 34% yield). LCMS (ESI, m/z)=448 [M+1]+.

Step 5: To a stirred solution of 4-bromo-N-[5-(2-chlorothiophen-3-yl)-1,3,4-thiadiazol-2-yl]-5-methoxy-6-oxopyran-2-carboxamide (“halo-pyrone reagent”) (160 mg, 0.357 mmol, 1.00 equiv) and 2-methoxyethan-1-amine (“amine reagent”) (32 mg, 0.43 mmol, 1.19 equiv) in N,N-dimethylformamide (DMF) (4 mL) was added CF₃COONa (160 mg, 1.18 mmol, 3.30 equiv), 2-dicyclohexylphosphino-2′,6′-diisopropoxybiphenyl (RuPhos) (63 mg, 0.14 mmol, 0.38 equiv), 1,8-diazabicyclo[5.4.0]undec-7-ene (DBU) (160 mg, 1.05 mmol, 2.95 equiv) and (2-dicyclohexylphosphino-2′,6′-diisopropoxy-1,1′-biphenyl)[2-(2′-amino-1,1′-biphenyl)]palladium(II) methanesulfonate (RuPhos Palladacycle Gen3) (63 mg, 0.075 mmol, 0.21 equiv) at room temperature. The resulting mixture was stirred for 1 h at 100° C. under N₂ (nitrogen gas). The resulting solids were filtered and washed with acetonitrile (MeCN) (10×3 mL). The filtrate was then concentrated under reduced pressure and the resulting residue was purified by C18 reverse phase flash chromatography (MeCN in water, 10% to 50% gradient in 10 min) to afford a crude product which was further purified by Prep-HPLC (Column: XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: 20% B to 28% B in 8 min, 28% B; Wave Length: 220 nm; RT1 (min): 6.58) to afford N-(5-(2-chlorothiophen-3-yl)-1,3,4-thiadiazol-2-yl)-3-methoxy-4-((2-methoxyethyl)amino)-2-oxo-2H-pyran-6-carboxamide (Compound 135) (4.4 mg, 2.8% yield). LCMS (ESI, m/z)=442.95 [M+1]+. ¹H NMR (400 MHz, DMSO-dr) δ 7.68-7.56 (m, 2H), 7.21 (s, 1H), 3.66 (s, 3H), 3.49-3.40 (m, 4H), 3.28 (s, 3H).

### Example 27: N-(5-(2-cyano-4-methylthiophen-3-yl)-1,3,4-thiadiazol-2-yl)-3-methoxy-4-((2-methoxyethyl)amino)-2-oxo-2H-pyran-6-carboxamide (Compound 137)

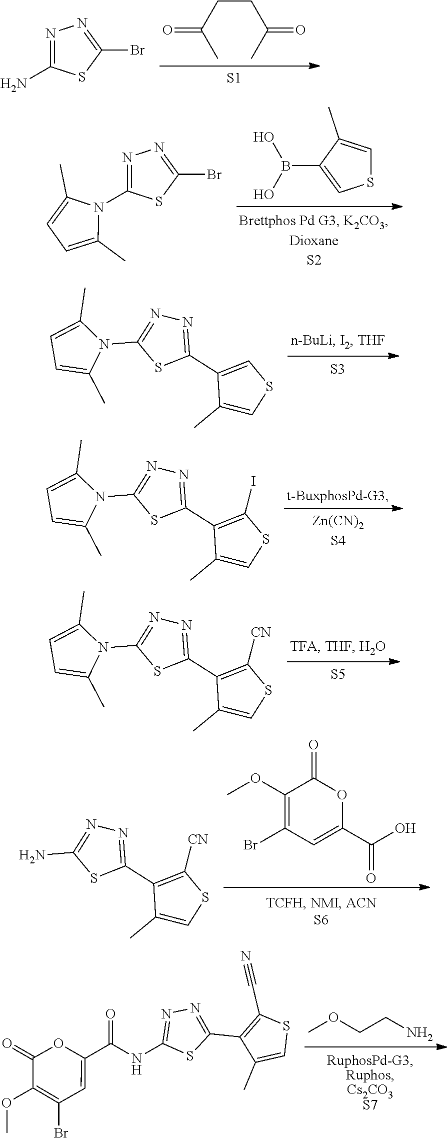

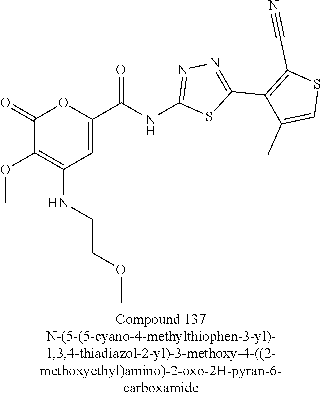 [SMILES: COCCNc1cc(C(=O)Nc2nnc(-c3c(C)csc3C#N)s2)oc(=O)c1OC]

Step 1: To a stirred solution of 5-bromo-1,3,4-thiadiazol-2-amine (10.0 g, 55.5 mmol, 1.00 equiv) and 2,5-hexanedione (8.93 g, 78.2 mmol, 1.41 equiv) in toluene (100 mL) was added tosic acid (TsOH) (1.92 g, 11.15 mmol, 0.20 equiv) at room temperature. The resulting mixture was stirred for 2 h at 120° C. The resulting mixture was concentrated under reduced pressure and diluted with water (300 mL). The mixture was extracted with ethyl acetate (EtOAc) (3×400 mL) and the combined organic layers were washed with water (3×100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (5:1), to afford 2-bromo-5-(2,5-dimethylpyrrol-1-yl)-1,3,4-thiadiazole (6.0 g, 42% yield).

Step 2: To a stirred solution of 2-bromo-5-(2,5-dimethylpyrrol-1-yl)-1,3,4-thiadiazole (1.00 g, 3.87 mmol, 1.00 equiv), 4-methylthiophen-3-ylboronic acid (0.83 g, 5.81 mmol, 1.50 equiv) in dioxane (10 mL) was added [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate (BrettPhos Pd G3) (1.40 g, 1.55 mmol, 0.40 equiv), K₂CO₃ (1.07 g, 7.75 mmol, 2.00 equiv) at room temperature. The resulting mixture was stirred overnight at 80° C. under N₂ (nitrogen gas) then concentrated under vacuum. The resulting residue was purified by C18 reverse phase flash chromatography (acetonitrile (MeCN):H₂O=1:1)) to provide 2-(2,5-dimethylpyrrol-1-yl)-5-(4-methylthiophen-3-yl)-1,3,4-thiadiazole (460 mg, 39% yield). LCMS (ESI, m/z)=276.0 [M+1]+.

Step 3: To a stirred solution of 2-(2,5-dimethylpyrrol-1-yl)-5-(4-methylthiophen-3-yl)-1,3,4-thiadiazole (400 mg, 1.45 mmol, 1.00 equiv) in tetrahydrofuran (THF) (60 mL) was added dropwise a solution of n-butyllithium (nBuLi) (0.87 mL, 2.18 mmol, 1.50 equiv) in THF (2.5 M) at −78° C. under N₂ (nitrogen gas). The reaction mixture was stirred at −78° C. for 1 h then a solution of I₂ (184 mg, 0.73 mmol, 0.50 equiv) in 2 mL THF was added dropwise and the mixture was stirred for another 1 h. The reaction was then quenched by the addition of sat. NH₄Cl (aq.) (10 mL) at 0° C. and extracted with ethyl acetate (EtOAc) (3×50 mL). The resulting mixture was concentrated under vacuum and the residue was purified by C18 reverse phase flash chromatography (acetonitrile (MeCN):H₂O=9:1) to provide 2-(2,5-dimethylpyrrol-1-yl)-5-(2-iodo-4-methylthiophen-3-yl)-1,3,4-thiadiazole (160 mg, 25% yield). LCMS (ESI, m/z)=402.0 [M+1]+.

Step 4: To a stirred solution of 2-(2,5-dimethylpyrrol-1-yl)-5-(2-iodo-4-methylthiophen-3-yl)-1,3,4-thiadiazole (120 mg, 0.31 mmol, 1.00 equiv) in N,N-dimethylacetamide (DMAC) (3 mL) was added Zn(CN)₂ (72 mg, 0.61 mmol, 2.00 equiv), XantPhos (266 mg, 0.46 mmol, 1.50 equiv). PdCl₂ (82 mg, 0.46 mmol, 1.50 equiv) and diisopropylethylamine (DIEA) (79 mg, 0.61 mmol, 2.0) equiv) at room temperature. The resulting mixture was stirred overnight at 100° C. under N₂ (nitrogen gas). The mixture was then diluted with water and extracted with ethyl acetate (EtOAc) (3×20 mL). The combined organic layers were concentrated under reduced pressure and the resulting residue purified by silica gel column chromatography, eluted with ethyl acetate/petroleum ether (EtOAc/PE) (3:1), to afford 3-[5-(2,5-dimethylpyrrol-1-yl)-1,3,4-thiadiazol-2-yl]-4-methylthiophene-2-carbonitrile (80 mg, 78% yield). LCMS (ESI, m/z)=301.0 [M+1]+.

Step 5: To a stirred solution of 3-[5-(2,5-dimethylpyrrol-1-yl)-1,3,4-thiadiazol-2-yl]-4-methylthiophene-2-carbonitrile (50 mg, 0.17 mmol, 1.00 equiv) in H₂O (0.7 mL) was added trifluoroacetic acid (TFA) (0.7 mL). The resulting mixture was stirred overnight at room temperature under air atmosphere. The mixture was then concentrated under reduced pressure to provide 3-(5-amino-1,3,4-thiadiazol-2-yl)-4-methylthiophene-2-carbonitrile (50 mg, 95% yield). LCMS (ESI, m/z)=223 [M+1]+.

Step 6: To a stirred solution of 3-(5-amino-1,3,4-thiadiazol-2-yl)-4-methylthiophene-2-carbonitrile (35 mg, 0.16 mmol, 1.00 equiv), 4-bromo-5-methoxy-6-oxopyran-2-carboxylic acid (product of Example 1, Part A, Step 4) (118 mg, 0.47 mmol, 3.00 equiv) in acetonitrile (MeCN) (1 mL) was added N-methylimidazole (NMI) (65 mg, 0.79 mmol, 5.00 equiv) and chloro-N,N,N′,N′-tetramethylformamidinium hexafluorophosphate (TCFH) (53 mg, 0.19 mmol, 1.20 equiv). The resulting mixture was stirred overnight at room temperature under air atmosphere then concentrated under reduced pressure. The residue was purified by C18 reverse phase flash chromatography (MeCN:H₂O=1:1) to provide 4-bromo-N-[5-(2-cyano-4-methylthiophen-3-yl)-1,3,4-thiadiazol-2-yl]-5-methoxy-6-oxopyran-2-carboxamide (30 mg, 38% yield). LCMS (ESI, m/z)=453 [M+1]+.

Step 7: N-(5-(2-Cyano-4-methylthiophen-3-yl)-1,3,4-thiadiazol-2-yl)-3-methoxy-4-((2-methoxyethyl)amino)-2-oxo-2H-pyran-6-carboxamide (Compound 137) was prepared according to Example 1, Part C, Step 2 using 4-bromo-N-[5-(2-cyano-4-methylthiophen-3-yl)-1,3,4-thiadiazol-2-yl]-5-methoxy-6-oxopyran-2-carboxamide as the “halo-pyrone reagent” and 2-methoxyethan-1-amine as the “amine reagent”. LCMS (ESI, m/z)=448 [M+1]+; ¹H NMR (400 MHz, Methanol-d₆) δ 7.65 (s, 1H), 7.37 (s, 1H), 3.79 (s, 3H), 3.62-3.58 (m, 4H), 3.41 (s, 3H), 2.47 (s, 3H).

### Example 28: N-[5-(4-cyanothiophen-3-yl)-1,3,4-thiadiazol-2-yl]-5-methoxy-4-[(2-methoxyethyl)amino]-6-oxopyran-2-carboxamide (Compound 138)

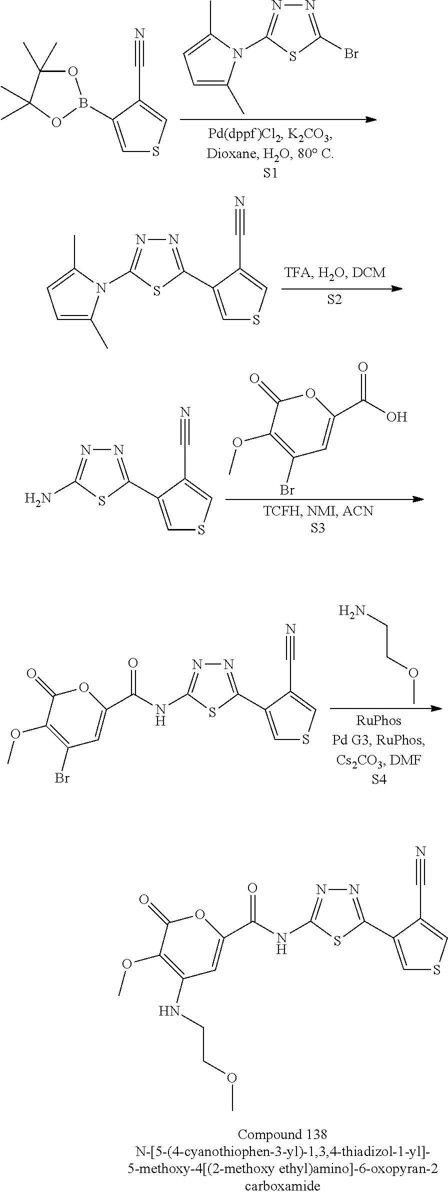

Step 1: To a stirred solution of 4-(4,4,5,5-tetramethyl-1,3,2-dioxaborolan-2-yl)thiophene-3-carbonitrile (210 mg, 0.89 mmol, 1.00 equiv) in dioxane (1.4 mL) and water (7 mL) was added 2-bromo-5-(2,5-dimethylpyrrol-1-yl)-1,3,4-thiadiazole (product of Example 27, Step 1) (272 mg, 1.05 mmol, 1.18 equiv), [1,1′-bis(diphenylphosphino)ferrocene]dichloropalladium(II) (Pd(dppf)Cl₂) (65 mg, 0.089 mmol, 0.10 equiv) and potassium carbonate (378 mg, 2.74 mmol, 3.06 equiv) at room temperature. The resulting mixture was stirred for 3 h at 90° C. under nitrogen (N₂) then cooled to room temperature and diluted with water (200 mL). The mixture was extracted with ethyl acetate (EtOAc) (3×200 mL) and the combined organic layers were washed with water (100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (5:1), to afford 4-[5-(2,5-dimethylpyrrol-1-yl)-1,3,4-thiadiazol-2-yl]thiophene-3-carbonitrile (128 mg, 38% yield). LCMS (ESI, m/z)=287 [M+1]+.

Step 2: To a stirred solution of 4-[5-(2,5-dimethylpyrrol-1-yl)-1,3,4-thiadiazol-2-yl]thiophene-3-carbonitrile (120 mg, 0.419 mmol, 1.00 equiv) in dichloromethane (DCM) (0.24 mL) was added water (0.24 mL) and trifluoroacetaldehyde (0.6 mL) at room temperature. The resulting mixture was stirred for 3 h at 50° C. The resulting solids were collected by filtration and washed with acetonitrile (MeCN) (0.5 mL) to provide 4-(5-amino-1,3,4-thiadiazol-2-yl)thiophene-3-carbonitrile (32 mg, 36% yield). LCMS (ESI, m/z)=209 [M+1]+.

Step 3: To a solution of 4-bromo-5-methoxy-6-oxopyran-2-carboxylic acid (product of Example 1, Part A, Step 4) (137 mg, 0.550 mmol, 1.00 equiv) in acetonitrile (MeCN) (4.7 mL) was added chloro-N,N,N′,N′-tetramethylformamidinium hexafluorophosphate (TCFH) (233 mg, 0.83 mmol, 1.51 equiv) and N-methylimidazole (NMI) (136 mg, 1.66 mmol, 3.01 equiv) at room temperature. The resulting mixture was stirred for 30 min at room temperature. To the above mixture was then added 4-(5-amino-1,3,4-thiadiazol-2-yl)thiophene-3-carbonitrile (103 mg, 0.495 mmol, 0.90 equiv) and the resulting mixture was stirred overnight at room temperature. The resulting solids were collected by filtration and washed with MeCN (1×2 mL) to provide 4-bromo-N-[5-(4-cyanothiophen-3-yl)-1,3,4-thiadiazol-2-yl]-5-methoxy-6-oxopyran-2-carboxamide (63 mg, 25% yield). LCMS (ESI, m/z)=439 [M+1]+.

Step 4: N-[5-(4-Cyanothiophen-3-yl)-1,3,4-thiadiazol-2-yl]-5-methoxy-4-[(2-methoxyethyl)amino]-6-oxopyran-2-carboxamide (Compound 138) was prepared according to Example 1, Part C, Step 2 using 4-bromo-N-[5-(4-cyanothiophen-3-yl)-1,3,4-thiadiazol-2-yl]-5-methoxy-6-oxopyran-2-carboxamide as the “halo-pyrone reagent” and 2-methoxyethan-1-amine as the “amine reagent”. LCMS (ES, m/z)=433.95 [M+1]+. ¹H NMR (400 MHz, DMSO-d6) δ 13.51 (br, 1H), 8.70 (s, 1H), 8.45 (s, 1H), 7.137 (s, 1H), 7.04 (s, 1H), 3.65 (s, 3H), 3.48-3.39 (m, 4H), 3.27 (s, 3H).

### Example 29: N-(5-(4-chloroisothiazol-5-yl)-1,3,4-thiadiazol-2-yl)-3-methoxy-4-((2-methoxyethyl)amino)-2-oxo-2H-pyran-6-carboxamide (Compound 139)

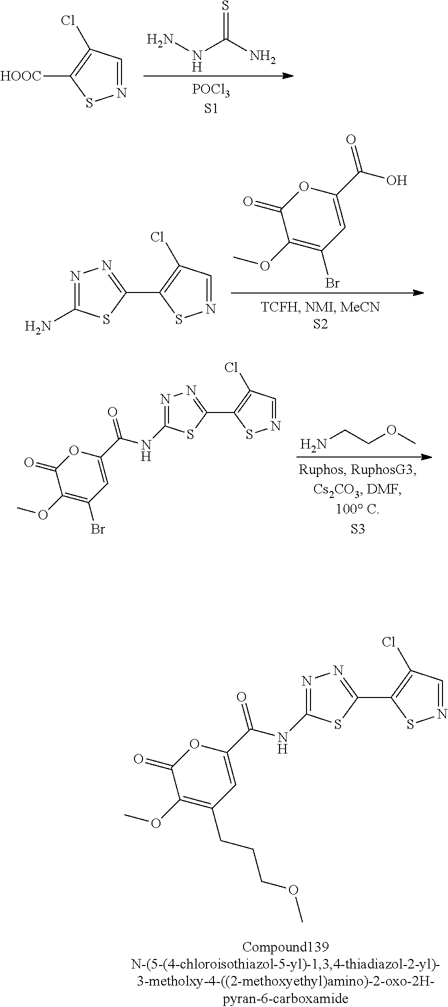

Step 1: To a stirred solution of 4-chloroisothiazole-5-carboxylic acid (600 mg, 3.67 mmol, 1.00 equiv) in POCl₃ (5 mL) was added thiosemicarbazide (540 mg, 5.926 mmol, 1.62 equiv) at room temperature. The resulting mixture was stirred for 5 h at room temperature then concentrated under vacuum and diluted with ethyl acetate (EtOAc) (200 mL). The resulting solution was neutralized to pH 8 with NaOH (aq, 1M) and extracted with ethyl acetate (EtOAc) (3×30 mL). The combined organic layers were washed with water and brine (3×5 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (5:1), to afford 5-(4-chloro-1,2-thiazol-5-yl)-1,3,4-thiadiazol-2-amine (200 mg, 25% yield). LCMS (EST, m/z)=218.9 [M+1]+.

Step 2: To a stirred solution of 5-(4-chloro-1,2-thiazol-5-yl)-1,3,4-thiadiazol-2-amine (70 mg, 0.32 mmol, 1.0 equiv) and 4-bromo-5-methoxy-6-oxopyran-2-carboxylic acid (product of Example 1, Part A, Step 4) (160 mg, 0.64 mmol, 2.01 equiv) in acetonitrile (MeCN) (5 ml) was added chloro-N,N,N′,N′-tetramethylformamidinium hexafluorophosphate (TCFH) (270 mg, 0.96 mmol, 3.01 equiv) and N-methylimidazole (NMI) (270 mg, 3.29 mmol, 10.3 equiv) at room temperature. The resulting mixture was stirred for 3 h at room temperature. The resulting solids were collected by filtration and washed with acetonitrile to provide 4-bromo-N-[5-(4-chloro-1,2-thiazol-5-yl)-1,3,4-thiadiazol-2-yl]-5-methoxy-6-oxopyran-2-carboxamide (100 mg, 69% yield). LCMS (ESI, m/z)=448.85 [M+1]+.

Step 3: N-(5-(4-chloroisothiazol-5-yl)-1,3,4-thiadiazol-2-yl)-3-methoxy-4-((2-methoxyethyl)amino)-2-oxo-2H-pyran-6-carboxamide (Compound 139) was prepared according to Example 1, Part C, Step 2 using 4-bromo-N-[5-(4-chloro-1,2-thiazol-5-yl)-1,3,4-thiadiazol-2-yl]-5-methoxy-6-oxopyran-2-carboxamide as “halo-pyrone reagent” and 2-methoxyethan-1-amine as the “amine reagent”. LCMS (ESI, m/z)=444.00 [M+1]+. ¹H NMR (400 MHz, DMSO-d6) δ 8.65 (s, 1H), 7.10 (s, 1H), 3.65 (s, 3H), 3.52-3.40 (m, 4H), 3.29 (s, 3H).

### Example 30: N-(5-(4-cyano-2-methylthiophen-3-yl)-1,3,4-thiadiazol-2-yl)-3-methoxy-4-((2-methoxyethyl)amino)-2-oxo-2H-pyran-6-carboxamide (Compound 140)

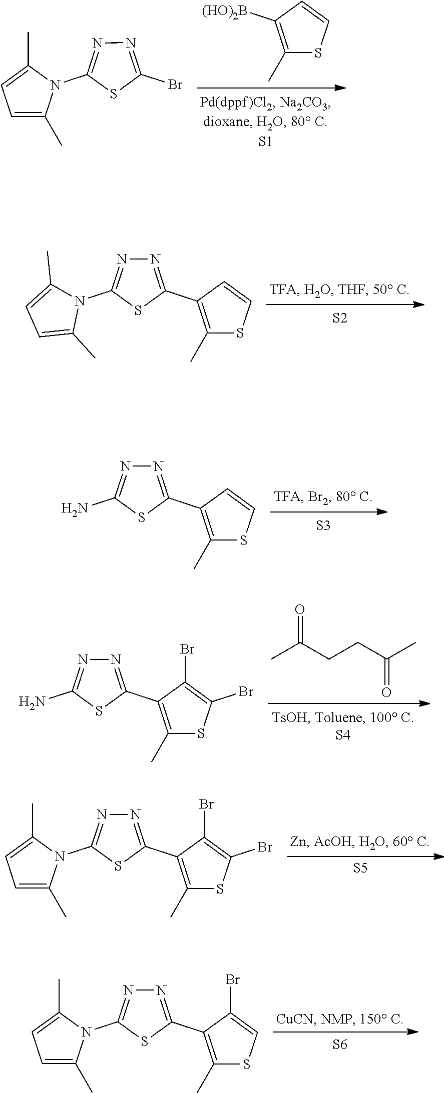

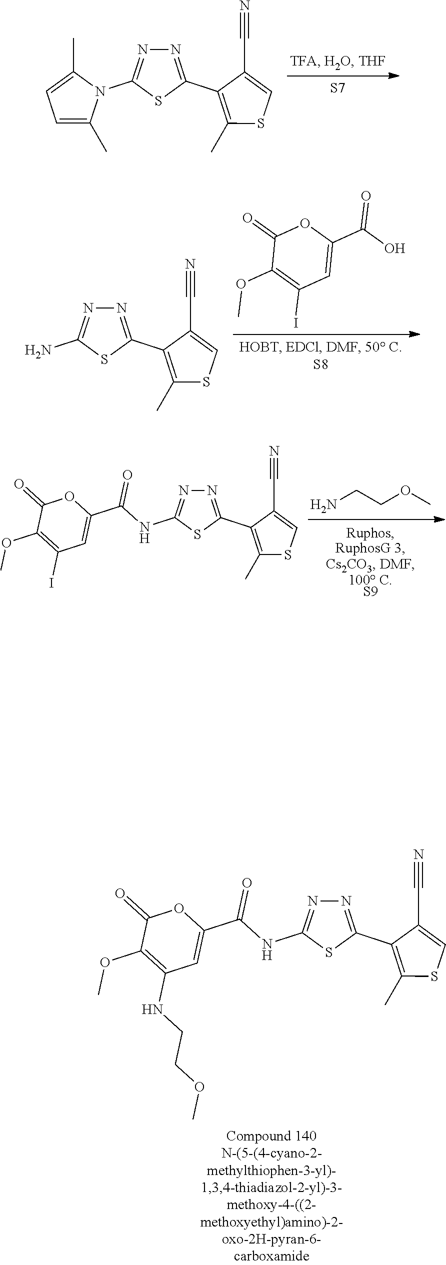

Step 1: To a stirred solution of 2-bromo-5-(2,5-dimethylpyrrol-1-yl)-1,3,4-thiadiazole (product of Example 27, Step 1) (2.50 g, 9.68 mmol, 1.00 equiv) and 2-methylthiophen-3-ylboronic acid (1.66 g, 11.7 mmol, 1.21 equiv) in dioxane (8 mL) were added [1,1′-bis(diphenylphosphino)ferrocene]dichloropalladium(II) (Pd(dppf)Cl₂) (0.80 g, 1.09 mmol, 0.11 equiv), H₂O (3 mL) and K₂CO₃ (2.70 g, 19.5 mmol, 2.02 equiv) dropwise room temperature under nitrogen (N₂). The resulting mixture was stirred for 4 h at 80° C. under N₂. The mixture was then diluted with water and extracted with ethyl acetate (EtOAc) (3×30 mL). The combined organic layers were washed with water and brine (3×5 mL), and dried over anhydrous Na₂SO₄. After filtration, the filtrate was concentrated under reduced pressure and the residue purified by silica gel column chromatography, eluted with PE/EtOAc (5:1), to afford 2-(2,5-dimethylpyrrol-1-yl)-5-(2-methylthiophen-3-yl)-1,3,4-thiadiazole (1.5 g, 56% yield). LCMS (ESI, m/z)=276 [M+1]+.

Step 2: To a stirred solution of 2-(2,5-dimethylpyrrol-1-yl)-5-(2-methylthiophen-3-yl)-1,3,4-thiadiazole (1.50 g, 5.45 mmol, 1.0) equiv) in H₂O (2 mL) and tetrahydrofuran (THF) (1 mL) was added trifluoroacetic acid (TFA) (2 mL) dropwise at room temperature. The resulting mixture was stirred for 5 h at 50° C. then concentrated under vacuum. The residue was then purified by C18 reverse phase flash chromatography (acetonitrile (MeCN) in water, 0% to 100% gradient in 10 min; detector, UV 254 nm) to provide 5-(2-methylthiophen-3-yl)-1,3,4-thiadiazol-2-amine (600 mg, 56% yield). LCMS (ESI, m/z)=198 [M+1]+.

Step 3: To a stirred solution of 5-(2-methylthiophen-3-yl)-1,3,4-thiadiazol-2-amine (500 mg, 2.54 mmol, 1.0) equiv) in trifluoroacetic acid (TFA) (20 mL) was added Br₂ (3.0 g, 19 mmol, 7.4 equiv) dropwise at room temperature. The resulting mixture was stirred overnight at 80° C. The mixture was then quenched with sat. NaHSO₃ and extracted with ethyl acetate (EtOAc) (3×30 mL). The combined organic layers were washed with water and brine (3×5 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (5:1), to afford 5-(4,5-dibromo-2-methylthiophen-3-yl)-1,3,4-thiadiazol-2-amine (800 mg, 89% yield). LCMS (ESI, w)=355 [M+1]+.

Step 4: To a stirred solution of 5-(4,5-dibromo-2-methylthiophen-3-yl)-1,3,4-thiadiazol-2-amine (700 mg, 1.97 mmol, 1.00 equiv) and 2,5-hexanedione (336 mg, 2.94 mmol, 1.49 equiv) in toluene (5 mL) was added tosic acid (TsOH) (112 mg, 0.65 mmol, 0.33 equiv) at room temperature. The resulting mixture was stirred overnight at 100° C. then concentrated under reduced pressure. The residue was then purified by silica gel column chromatography, eluted with petroleum ether/ethyl acetate (PE/EtOAc) (5:1), to afford 2-(4,5-dibromo-2-methylthiophen-3-yl)-5-(2,5-dimethylpyrrol-1-yl)-1,3,4-thiadiazole (600 mg, 70% yield). LCMS (EST, m/z)=433 [M+1]+.

Step 5: To a stirred solution of 2-(4,5-dibromo-2-methylthiophen-3-yl)-5-(2,5-dimethylpyrrol-1-yl)-1,3,4-thiadiazole (600 mg, 1.38 mmol, 1.00 equiv) in acetic acid (AcOH) (5 mL) and H₂O (5 mL) was added Zn (540 mg, 8.26 mmol, 5.96 equiv) at room temperature. The resulting mixture was stirred for 3 h at room temperature. The mixture was then diluted with water and extracted with ethyl acetate (EtOAc) (3×50 mL). The combined organic layers were washed with water and brine (3×5 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (5:1), to afford 2-(4-bromo-2-methylthiophen-3-yl)-5-(2,5-dimethylpyrrol-1-yl)-1,3,4-thiadiazole (440 mg, 90/o yield). LCMS (ESI, m/z)=355 [M+1]+.

Step 6: To a stirred solution of 2-(4-bromo-2-methylthiophen-3-yl)-5-(2,5-dimethylpyrrol-1-yl)-1,3,4-thiadiazole (440 mg, 1.24 mmol, 1.00 equiv) in NMP (5 mL) was added CuCN (230 mg, 2.57 mmol, 2.07 equiv) at room temperature. The resulting mixture was stirred for 3 h at 150° C. The mixture was then extracted with EtOAc (3×30 mL). The combined organic layers were washed with water and brine (3×5 mL), and dried over anhydrous Na₂SO₄. After filtration, the filtrate was concentrated under reduced pressure and the resulting residue was purified by silica gel column chromatography, eluted with petroleum ether/ethyl acetate (PE/EtOAc) (5:1), to afford 4-[5-(2,5-dimethylpyrrol-1-yl)-1,3,4-thiadiazol-2-yl]-5-methylthiophene-3-carbonitrile (300 mg, 80% yield). LCMS (ESI, m/z)=301 [M+1]+.

Step 7: To a stirred solution of 4-[5-(2,5-dimethylpyrrol-1-yl)-1,3,4-thiadiazol-2-yl]-5-methylthiophene-3-carbonitrile (300 mg, 0.99 mmol, 1.00 equiv) in H₂O (2 mL) and tetrahydrofuran (THF) (1 mL) was added trifluoroacetic acid (TFA) (2 mL) dropwise at room temperature. The resulting mixture was stirred for 2 h at 50° C. then concentrated under vacuum. The resulting residue was then purified by C18 reverse phase flash chromatography (acetonitrile (MeCN) in Water, 0% to 100% gradient in 10 min; detector, UV 254 nm) to provide 4-(5-amino-1,3,4-thiadiazol-2-yl)-5-methylthiophene-3-carbonitrile (130 mg, 58% yield). LCMS (ESI, m/z)=222 [M+1]+.

Step 8: To a stirred solution of 4-iodo-5-methoxy-6-oxopyran-2-carboxylic acid (product of Example 6, Step 3) (192 mg, 0.649 mmol, 1.07 equiv) and 4-(5-amino-1,3,4-thiadiazol-2-yl)-5-methylthiophene-3-carbonitrile (120 mg, 0.608 mmol, 1.00 equiv) in N,N-dimethylformamide (DMF) (5 mL) was added hydroxybenzotriazole (HOBT) (102 mg, 0.755 mmol, 1.24 equiv) and 1-ethyl-3-(3-dimethylaminopropyl)carbodiimide (EDCI) (141 mg, 0.736 mmol, 1.21 equiv) at room temperature. The resulting mixture was stirred for 3 h at 50° C. The resulting solids were collected by filtration and washed with DMF (3×1 mL) to provide N-[5-(4-cyano-2-methylthiophen-3-yl)-1,3,4-thiadiazol-2-yl]-4-iodo-5-methoxy-6-oxopyran-2-carboxamide (200 mg, 69% yield). LCMS (EST, m/z)=50) [M+1]+.

Step 9: N-(5-(4-Cyano-2-methylthiophen-3-yl)-1,3,4-thiadiazol-2-yl)-3-methoxy-4-((2-methoxyethyl)amino)-2-oxo-2H-pyran-6-carboxamide (Compound 140) was prepared according to Example 1, Part C, Step 2 using 2-methoxyethan-1-amine as the “amine reagent” and N-[5-(4-cyano-2-methylthiophen-3-yl)-1,3,4-thiadiazol-2-yl]-4-iodo-5-methoxy-6-oxopyran-2-carboxamide as the “halo-pyrone reagent”. LCMS (EST m/z)=448.05 [M+1]+. ¹H NMR (300 MHz, DMSO-d6) S 8.52 (s, 1H), 7.20 (s, 1H), 6.91 (s, 1H), 3.66 (s, 3H), 3.52-3.42 (m, 4H), 3.29 (s, 3H), 2.63 (s, 3H).

### Example 31: N-(5-(5-ethyl-1H-pyrazol-1-yl)-1,3,4-thiadiazol-2-yl)-3-methoxy-4-((2-methoxyethyl)amino)-2-oxo-2H-pyran-6-carboxamide (Compound 141)

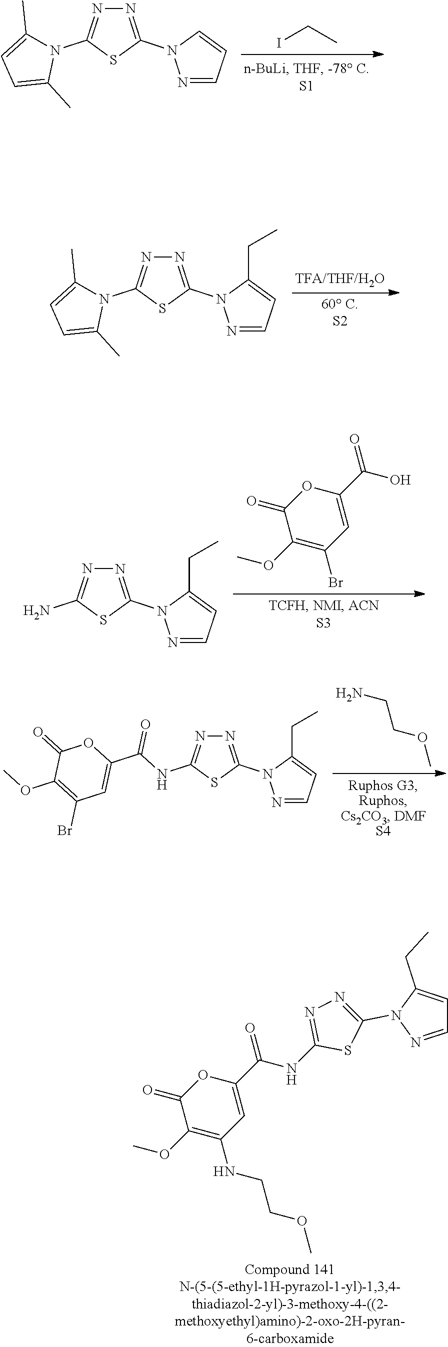

Step 1: Into a solution of 2-(2,5-dimethylpyrrol-1-yl)-5-(pyrazol-1-yl)-1,3,4-thiadiazole (product of Example 1, Part B, Step 2) (6.0 g, 24 mmol, 1.0 equiv) in tetrahydrofuran (THF) (150 mL) was added n-butyl lithium (n-BuLi) (12 mL, 30 mmol, 1.2 equiv) in hexanes (2.5 M) dropwise over 15 min at −78° C. under N₂ (nitrogen gas). The resulting mixture was stirred for an additional 1.5 h at −78° C. To the mixture was then added ethyl iodide (4500 mg, 28.85 mmol, 1.18 equiv) dropwise at −78° C. under N₂. The resulting mixture was stirred for a further 1.5 h at room temperature. The reaction was then quenched with sat. NH₄Cl (aq.) at 0° C. The aqueous layer was extracted with ethyl acetate (EtOAc) (3×300 mL). The combined organic layers were concentrated under reduced pressure and the residue was purified by silica gel column chromatography, eluted with petroleum ether/ethyl acetate (PE/EtOAc) (9:1), to afford 2-(2,5-dimethylpyrrol-1-yl)-5-(5-ethylpyrazol-1-yl)-1,3,4-thiadiazole (5100 mg, 72% yield). LCMS (ESI, m/z)=274.0 [M+1]+.

Step 2: Into a solution of 2-(2,5-dimethylpyrrol-1-yl)-5-(5-ethylpyrazol-1-yl)-1,3,4-thiadiazole (50001 mg, 19.28 mmol, 1.00 equiv) in THF (10 mL) were added H₂O (20 mL) and trifluoroacetic acid (TFA) (20 mL) at room temperature. The resulting mixture was stirred for 4 h at 60° C. then concentrated under reduced pressure. The residue was purified by C18 reverse phase flash chromatography (acetonitrile (MeCN) in Water, 10% to 50% gradient in 15 min detector, UV 254 nm) to afford 5-(5-ethylpyrazol-1-yl)-1,3,4-thiadiazol-2-amine (3000 mg, 82% yield). LCMS (ESI, m/z)=196.0 [M+1]+.

Step 3: Into a solution of 4-bromo-5-methoxy-6-oxopyran-2-carboxylic acid (product of Example 1, Part A, Step 4) (200 mg, 0.80 mmol, 1.00 equiv) in acetonitrile (MeCN) (5 mL) was added chloro-N,N,N′,N′-tetramethylformamidinium hexafluorophosphate (TCFH) (270 mg, 0.96 mmol, 1.20 equiv), N-methylimidazole (NMI) (132 mg, 1.61 mmol, 2.00 equiv) and 5-(5-ethylpyrazol-1-yl)-1,3,4-thiadiazol-2-amine (157 mg, 0.80 mmol, 1.0 equiv) at room temperature. The resulting mixture was stirred overnight at room temperature. The resulting solids were collected by filtration and washed with acetonitrile. The solids were then purified by trituration with acetonitrile and water (15 mL) to provide 4-bromo-N-[5-(5-ethylpyrazol-1-yl)-1,3,4-thiadiazol-2-yl]-5-methoxy-6-oxopyran-2-carboxamide (150 mg, 39% yield). LCMS (EST, m/z)=425.9 [M+1]+.

Step 4: N-(5-(5-Ethyl-1H-pyrazol-1-yl)-1,3,4-thiadiazol-2-yl)-3-methoxy-4-((2-methoxyethyl)amino)-2-oxo-2H-pyran-6-carboxamide (Compound 141) was prepared according to Example 1, Part C, Step 2 using 2-methoxyethan-1-amine as the “amine reagent” and 4-bromo-N-[5-(5-ethylpyrazol-1-yl)-1,3,4-thiadiazol-2-yl]-5-methoxy-6-oxopyran-2-carboxamide as the “halo-pyrone reagent”. LCMS (ESI, m/z)=421.10 [M+1]+. 1H NMR (400 MHz, DMSO-d6) δ 7.70 (d, J=1.6 Hz, 1H), 7.22 (s, 1H), 6.38 (d, J=1.6 Hz, 1H), 3.64 (s, 3H), 3.50-3.41 (m, 4H), 3.26 (s, 3H), 3.05 (q, J=7.2 Hz, 2H), 1.23 (t, J=7.2 Hz, 3H).

### Example 32: (R)—N-(5-(5-(hydroxymethyl)-1H-pyrazol-1-yl)-1,3,4-thiadiazol-2-yl)-3-methoxy-4-((1-methoxypropan-2-yl)amino)-2-oxo-2H-pyran-6-carboxamide (Compound 142)

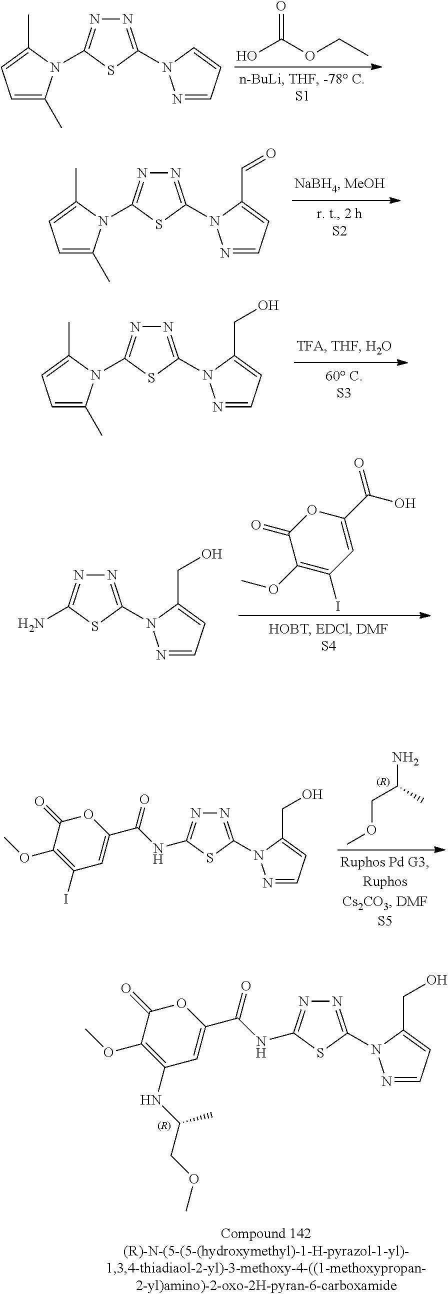

Step 1: Into a solution of 2-(2,5-dimethylpyrrol-1-yl)-5-(pyrazol-1-yl)-1,3,4-thiadiazole (product of Example 1, Part B, Step 2) (5.0 g, 20 mmol, 1.0 equiv) in tetrahydrofuran (THF) (100 mL) was added n-butyl lithium (n-BuLi) (8.97 mL, 22.4 mmol, 1.10 equiv) dropwise over 5 min at −78° C. The resulting mixture was stirred for 1 h at −78° C. To the mixture was then added ethyl formate (4.53 g, 61.1 mmol, 3.00 equiv) dropwise over 5 min at −78° C. The mixture was stirred for an additional 1 h at −78° C. then quenched with sat. NH₄Cl (aq.) at 0° C. The aqueous layer was extracted with ethyl acetate (EtOAc) (3×100 mL). The resulting mixture was concentrated under reduced pressure and the resulting residue was purified by silica gel column chromatography, eluted with petroleum ether/ethyl acetate (PE/EtOAc) (8:1), to afford 2-[5-(2,5-dimethylpyrrol-1-yl)-1,3,4-thiadiazol-2-yl]pyrazole-3-carbaldehyde (3.5 g, 56% yield). LCMS (ESI, m/z)=274.2 [M+1]+.

Step 2: Into a solution of 2-[5-(2,5-dimethylpyrrol-1-yl)-1,3,4-thiadiazol-2-yl]pyrazole-3-carbaldehyde (3.5 g, 13 mmol, 1.0 equiv) in methanol (MeOH) (50 mL) was added NaBH₄ (2.42 g, 64.0 mmol, 5.00 equiv) at 0° C. The resulting mixture was stirred for 2 h at room temperature then quenched with HCl (1M) at 0° C. The aqueous layer was extracted with EtOAc (3×60 mL) and concentrated under reduced pressure. The residue was purified by silica gel column chromatography, eluted with petroleum ether/ethyl acetate (PE/EtOAc) (6:1), to afford {2-[5-(2,5-dimethylpyrrol-1-yl)-1,3,4-thiadiazol-2-yl]pyrazol-3-yl}methanol (3.3 g, 84% yield). LCMS (ESI, m/z)=276.0 [M+1]+.

Step 3: Into a solution of {2-[5-(2,5-dimethylpyrrol-1-yl)-1,3,4-thiadiazol-2-yl]pyrazol-3-yl}methanol (3.3 g, 12 mmol, 10 equiv) in tetrahydrofuran (THF) (16 mL) was added H₂O (16 mL) and trifluoroacetic acid (TFA) (20 mL) at room temperature. The resulting mixture was stirred for 5 h at 60° C. then concentrated under reduced pressure. The residue was then purified by C18 reverse phase flash chromatography (acetonitrile (MeCN) in Water, 10% to 50% gradient in 10 min; detector, UV 254 nm) to provide [2-(5-amino-1,3,4-thiadiazol-2-yl)pyrazol-3-yl]methanol (1.5 g, 57% yield). LCMS (ESI, m/z)=198.0 [M+1]+.

Step 4: Into a solution of 4-iodo-5-methoxy-6-oxopyran-2-carboxylic acid (product of Example 6, Step 3) (2.25 g, 7.61 mmol, 1.00 equiv) in N,N-dimethylformamide (DMF) (20 mL) was added hydroxybenzotriazole (HOBT) (1.54 g, 11.4 mmol, 1.50 equiv), 1-ethyl-3-(3-dimethylaminopropyl)carbodiimide (EDCI) (2.92 g, 15.2 mmol, 2.00 equiv) and [2-(5-amino-1,3,4-thiadiazol-2-yl)pyrazol-3-yl]methanol (1.5 g, 7.6 mmol, 1.0 equiv) at room temperature. The resulting mixture was stirred for 1 h at room temperature. The reaction was then quenched with water at room temperature. The resulting solids were collected by filtration and washed with water. The filtrate was concentrated under reduced pressure to provide N-(5-[5-(hydroxymethyl)pyrazol-1-yl]-1,3,4-thiadiazol-2-yl)-4-iodo-5-methoxy-6-oxopyran-2-carboxamide (2.0 g, 50% yield). LCMS (ESI, m/z)=475.9 [M+1]+.

Step 5: (R)—N-(5-(5-(Hydroxymethyl)-1H-pyrazol-1-yl)-1,3,4-thiadiazol-2-yl)-3-methoxy-4-((1-methoxypropan-2-yl)amino)-2-oxo-2H-pyran-6-carboxamide (Compound 142) was prepared according to Example 1, Part C, Step 2 using (R)-1-methoxypropan-2-amine as the “amine reagent” and N-{5-[5-(hydroxymethyl)pyrazol-1-yl]-1,3,4-thiadiazol-2-yl}-4-iodo-5-methoxy-6-oxopyran-2-carboxamide as the “halo-pyrone reagent”. LCMS (ESI, m/z)=437.10 [M+1]+. ¹H NMR (400 MHz, DMSO-d6) δ 7.83 (d, J=1.6 Hz, 1H), 7.40 (s, 1H), 6.57 (d, J=1.6 Hz, 1H), 4.94 (d, J=1.0 Hz, 2H), 4.02-3.97 (m, 1H), 3.67 (s, 3H), 3.43-3.32 (m, 2H), 3.27 (s, 3H), 1.17 (d, J=6.4 Hz, 3H).

### Example 33: N-(5-(5-(difluoromethyl)-1H-pyrazol-1-yl)-1,3,4-thiadiazol-2-yl)-3-methoxy-4-((2-methoxyethyl)amino)-2-oxo-2H-pyran-6-carboxamide (Compound 144)

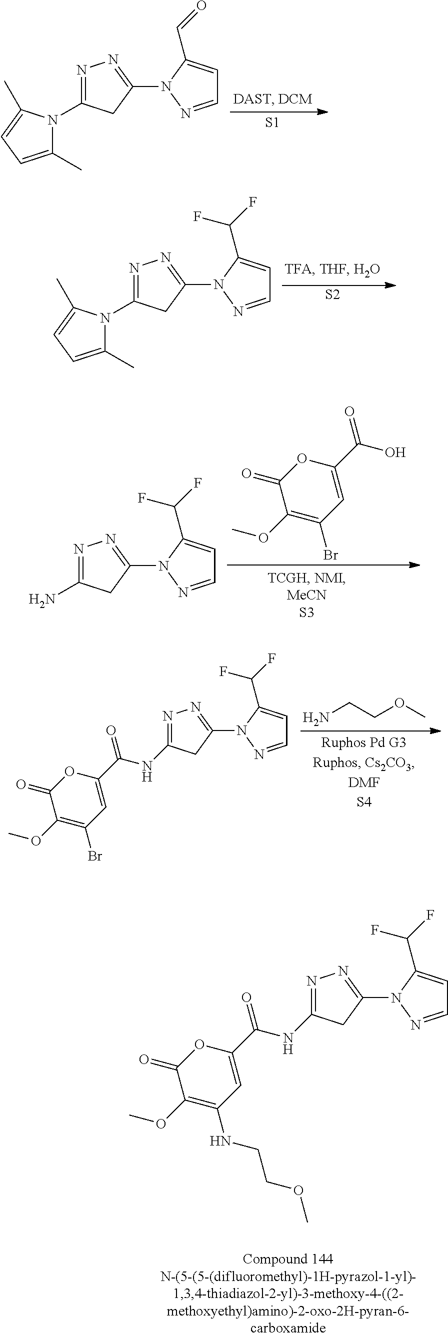

Step 1: To a stirred solution of 2-[5-(2,5-dimethylpyrrol-1-yl)-1,3,4-thiadiazol-2-yl]pyrazole-3-carbaldehyde (product of Step 1 of Example 32) (700 mg, 2.56 mmol, 1.00 equiv) in dichloromethane (DCM) (5 mL) was added diethylaminosulfur trifluoride (DAST) (826 mg, 5.12 mmol, 2.00 equiv) at 0° C. The resulting mixture was stirred for 2 h at room temperature under air atmosphere. The reaction was then quenched by the addition of water (2 mL) at room temperature and extracted with ethyl acetate (EtOAc) (3×50 mL). The combined organic layers were concentrated under reduced pressure and the residue purified by silica gel column chromatography, eluted with petroleum ether/ethyl acetate (PE/EtOAc) (5:1), to afford 2-[5-(difluoromethyl)pyrazol-1-yl]-5-(2,5-dimethylpyrrol-1-yl)-1,3,4-thiadiazole (300 mg, 40% yield).

Step 2: Into a solution of 2-[5-(difluoromethyl)pyrazol-1-yl]-5-(2,5-dimethylpyrrol-1-yl)-1,3,4-thiadiazole (300 mg, 1.02 mmol, 1.00 equiv) in tetrahydrofuran (THF) (1 mL) and H₂O (0.5 mL) was added trifluoroacetic acid (TFA) (0.5 mL) at 0° C. The resulting mixture was stirred for 2 h at 60° C. under air atmosphere. The mixture was then concentrated under reduced pressure and the residue purified by silica gel column chromatography, eluted with petroleum ether/ethyl acetate (PE/EtOAc) (5:1), to afford 5-[5-(difluoromethyl)pyrazol-1-yl]-1,3,4-thiadiazol-2-amine (190 mg, 86% yield). LCMS (ESI, m/z)=218.0 [M+1]+.

Step 3: To a stirred solution of 4-bromo-5-methoxy-6-oxopyran-2-carboxylic acid (product of Example 1, Part A, Step 4) (218 mg, 0.87 mmol, 1.00 equiv) in acetonitrile (MeCN) (1 mL) was added chloro-N,N,N′,N′-tetramethylformamidinium hexafluorophosphate (TCFH) (294 mg, 1.05 mmol, 1.20 equiv), N-methylimidazole (NMI) (215 mg, 2.62 mmol, 3.00 equiv) and 5-[5-(difluoromethyl)pyrazol-1-yl]-1,3,4-thiadiazol-2-amine (190 mg, 0.875 mmol, 1.00 equiv) at room temperature. The resulting mixture was stirred for an additional 50 min at room temperature. The resulting solids were collected by filtration and washed with MeCN (3×10 mL) to provide 4-bromo-N-{5-[5-(difluoromethyl)pyrazol-1-yl]-1,3,4-thiadiazol-2-yl}-5-methoxy-6-oxopyran-2-carboxamide (220 mg, 56% yield). LCMS (ESI, m/z)=447.9 [M+1]+.

Step 4: N-(5-(5-(Difluoromethyl)-1H-pyrazol-1-yl)-1,3,4-thiadiazol-2-yl)-3-methoxy-4-((2-methoxyethyl)amino)-2-oxo-2H-pyran-6-carboxamide (Compound 144) was prepared according to Example 1, Part C, Step 2 using 4-bromo-N-{5-[5-(difluoromethyl)pyrazol-1-yl]-1,3,4-thiadiazol-2-yl}-5-methoxy-6-oxopyran-2-carboxamide as “halo-pyrone reagent” and 2-methoxyethan-1-amine as the “amine reagent”. LCMS (ESI, m/z)=443.1 [M+1]+. ¹H NMR (400 MHz, DMSO-d6) δ 7.94 (s, 1H), 7.72 (t, J=50.2 Hz, 1H), 7.15 (s, 1H), 6.97 (s, 1H), 6.82 (s, 1H), 3.67 (s, 3H), 3.48 (s, 4H), 3.29 (s, 3H).

### Example 34: N-(5-(3-cyanothiophen-2-yl)-1,3,4-thiadiazol-2-yl)-3-methoxy-4-((2-methoxyethyl)amino)-2-oxo-2H-pyran-6-carboxamide (Compound 145)

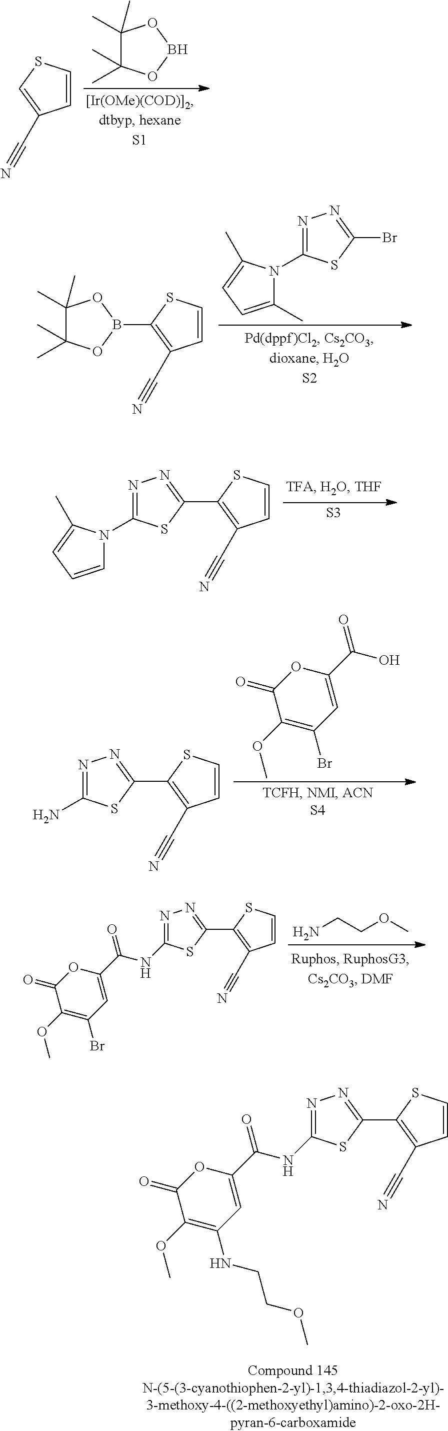

Step 1: A solution of thiophene-3-carbonitrile (500 mg, 4.58 mmol, 1.00 equiv) and 4,4′-Di-tert-butyl-2,2′-bipyridine (dtbpy) (123 mg, 0.458 mmol, 0.10 equiv). Bis(1,5-cyclooctadiene)di-mu-methoxydiiridium(I) [Ir(OMe)(COD)]2 (304 mg, 0.458 mmol, 0.10 equiv) in hexanes (2 mL) was stirred for 2 min at room temperature under N₂ (nitrogen gas). To the above mixture was added 4,4,5,5-tetramethyl-1,3,2-dioxaborolane (586 mg, 4.58 mmol, 1.00 equiv) dropwise over 2 min at 0° C. The resulting mixture was stirred for an additional 48 h at room temperature then concentrated under reduced pressure. The residue was then purified by silica gel column chromatography, eluted with petroleum ether/ethyl acetate (PE/EtOAc) (1:1), to afford 2-(4,4,5,5-tetramethyl-1,3,2-dioxaborolan-2-yl)thiophene-3-carbonitrile (220 mg, 20% yield).

Steps 2-5: N-(5-(3-Cyanothiophen-2-yl)-1,3,4-thiadiazol-2-yl)-3-methoxy-4-((2-methoxyethyl)amino)-2-oxo-2H-pyran-6-carboxamide (Compound 145) was prepared according to Example 28 steps 1-4 using 2-(4,4,5,5-tetramethyl-1,3,2-dioxaborolan-2-yl)thiophene-3-carbonitrile in place of 4-(4,4,5,5-tetramethyl-1,3,2-dioxaborolan-2-yl)thiophene-3-carbonitrile to provide 2-(5-amino-1,3,4-thiadiazol-2-yl)thiophene-3-carbonitrile (product of Step 3 of this Example), followed by coupling with 4-bromo-3-methoxy-2-oxo-2H-pyran-6-carboxylic acid to provide 4-bromo-N-(5-(3-cyanothiophen-2-yl)-1,3,4-thiadiazol-2-yl)-3-methoxy-2-oxo-2H-pyran-6-carboxamide as the “halo-pyrone reagent” and 2-methoxyethan-1-amine as the “amine reagent”. LCMS (EST, m/z)=434.00 [M+1]+. 1H NMR (400 MHz, DMSO-d6) δ 8.54 (s, 1H), 7.80 (s, 1H), 7.06 (s, 1H), 6.74 (t, J=5.6 Hz, 1H), 3.65 (s, 3H), 3.48-3.46 (m, 4H), 3.29 (s, 3H).

### Example 35: N-[5-(5-chloropyrazol-1-yl)-1,3,4-thiadiazol-2-yl]-5-methoxy-4-[(2-methoxyethyl)amino]-6-oxopyran-2-carboxamide (Compound 148)

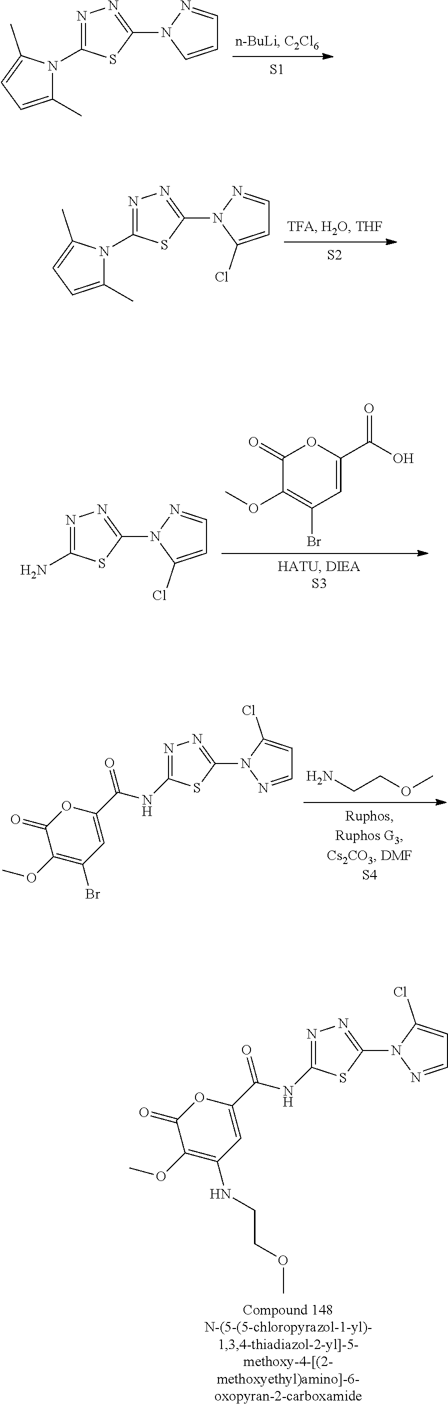

Step 1: A solution of 2-(2,5-dimethylpyrrol-1-yl)-5-(pyrazol-1-yl)-1,3,4-thiadiazole (product of Example 1, Part B, Step 2) (1000 mg, 4.077 mmol, 1.00 equiv) in tetrahydrofuran (THF) was added dropwise n-butyl lithium (n-BuLi) (2.4 mL, 6.0 mmol, 1.5 equiv) at −78° C. under N₂ (nitrogen gas). The solution was stirred at −78° C. for 1 h followed by the addition of hexachloroethane (C₂Cl₆) (0000 mg, 4.22 mmol, 1.04 equiv) dropwise at −78° C. The resulting mixture was stirred for 2 h at room temperature under N₂ (nitrogen gas) then quenched with sat. NH₄Cl (aq.) at 0° C. The aqueous layer was extracted with ethyl acetate (EtOAc) (3×30 mL). The extracts were concentrated under reduced pressure and the resulting residue was purified by silica gel column chromatography, eluted with petroleum ether/ethyl acetate (PE/EtOAc) (9:1), to afford 2-(5-chloropyrazol-1-yl)-5-(2,5-dimethylpyrrol-1-yl)-1,3,4-thiadiazole (880 mg, 76% yield). LCMS (ESI, m/z)=280.0 [M+1]+.

Step 2: Into a solution of 2-(5-chloropyrazol-1-yl)-5-(2,5-dimethylpyrrol-1-yl)-1,3,4-thiadiazole (880 mg, 3.15 mmol, 1.00 equiv) in THE (1 mL) and H₂O (2 mL) was added trifluoroacetic acid (TFA) (2 mL, 27 mmol, 8.6 equiv) at room temperature. The resulting mixture was stirred for 3 h at 60° C. then concentrated under reduced pressure. The residue was purified by C18 reverse phase flash chromatography (acetonitrile (MeCN) in water, 10% to 30% gradient in 7 min; detector, UV 254 nm) to afford 5-(5-chloropyrazol-1-yl)-1,3,4-thiadiazol-2-amine (390 mg, 61% yield). LCMS (ESI, m/z)=202.0 [M+1]+.

Step 3: Into a solution of 4-bromo-5-methoxy-6-oxopyran-2-carboxylic acid (product of Example 1, Part A, Step 4) (300) mg, 1.21 mmol, 1.00 equiv) and 5-(5-chloropyrazol-1-yl)-1,3,4-thiadiazol-2-amine (270 mg, 1.34 mmol, 1.11 equiv) in N,N-dimethylformamide (DMF) (3 mL) was added 1-((dimethylamino)(dimethyliminio)methyl)-1H-[1,2,3]triazolo[4,5-b]pyridine 3-oxide hexafluorophosphate(V) (HATU) (692 mg, 1.82 mmol, 1.51 equiv) and diisopropylethylamine (DIEA) (469 mg, 3.63 mmol, 3.01 equiv) at 0° C. under N₂ (nitrogen gas). The resulting mixture was stirred at room temperature for 16 h under N₂ then diluted with water and extracted with ethyl acetate (EtOAc) (3×100 mL). The combined organic layers were dried over anhydrous MgSO₄. After filtration, the filtrate was concentrated under reduced pressure and the residue was purified by silica gel column chromatography, eluted with methanol (MeOH)/dichloromethane (DCM) (1:4), to afford 4-bromo-N-[5-(5-chloropyrazol-1-yl)-1,3,4-thiadiazol-2-yl]-5-methoxy-6-oxopyran-2-carboxamide (also referred to herein as 4-bromo-N-(5-(5-chloro-1H-pyrazol-1-yl)-1,3,4-thiadiazol-2-yl)-3-methoxy-2-oxo-2H-pyran-6-carboxamide) (400 mg, 69% yield). LCMS (ES, m/z)=432.2 [M+1]+.

Step 4: N-[5-(5-Chloropyrazol-1-yl)-1,3,4-thiadiazol-2-yl]-5-methoxy-4-[(2-methoxyethyl)amino]-6-oxopyran-2-carboxamide (Compound 148) was prepared according to Example 1, Part C, Step 2 using 4-bromo-N-(5-(5-chloro-1H-pyrazol-1-yl)-1,3,4-thiadiazol-2-yl)-3-methoxy-2-oxo-2H-pyran-6-carboxamide as “halo-pyrone reagent” and 2-methoxyethan-1-amine as the “amine reagent”. LCMS (ESI, m/z)=427.0 [M+1]+. ¹H NMR (400 MHz, DMSO-d₆) δ 13.52 (br, 1H), 8.02 (d, J=2.0 Hz, 1H), 7.41 (s, 1H), 7.08 (br, 1H), 6.85 (d, J=2.0 Hz, 1H), 3.69 (s, 3H), 3.51-3.48 (m, 4H), 3.29 (s, 3H).

### Example 36: 4-(bicyclo[1,1,1]pentan-1-ylamino)-(3-methoxy-2,2-dimethylpropoxy)-N-(5-(5-methyl-1H-pyrazol-1-yl)-1,3,4-thiadiazol-2-yl)-2-oxo-2H-pyran-6-carboxamide (Compound 149)

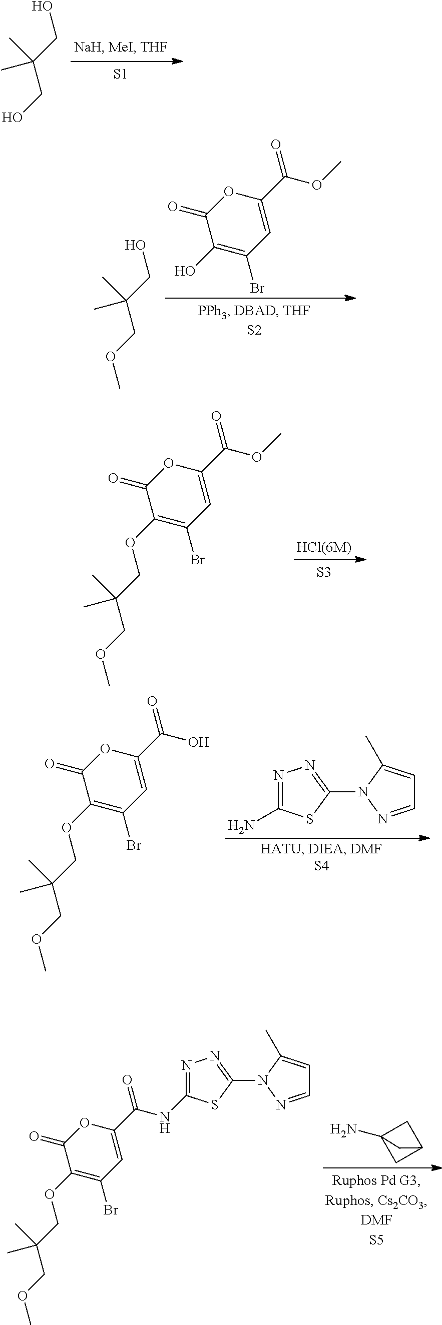

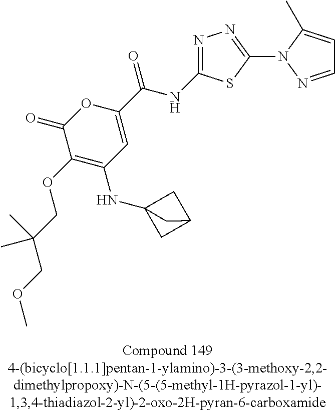 [SMILES: COCC(C)(C)COc1c(NC23CC(C2)C3)cc(C(=O)Nc2nnc(-n3nccc3C)s2)oc1=O]

Step 1: To a stirred solution of 2,2-dimethylpropane-1,3-diol (10.0 g, 96.0 mmol, 1.00 equiv) in tetrahydrofuran (THF) (100 mL) was added NaH (3.60 g, 150 mmol, 1.56 equiv) at 0° C. The resulting mixture was stirred for 30 min at 0° C. then methyl iodide (CH₃I) (16.0 g, 113 mmol, 1.17 equiv) added at 0° C. The resulting mixture was stirred overnight at room temperature then quenched with sat. NH₄Cl (aq.) at room temperature. The resulting mixture was poured into water and extracted with ethyl acetate (EtOAc) (3×200 mL). The combined organic layers were washed with water (3×100 mL) and brine (3×100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (4:1), to afford 3-methoxy-2,2-dimethylpropan-1-ol (2.9 g, 25% yield). LCMS (ES, m/z)=119 [M+1]+.

Step 2: To a stirred solution of 3-methoxy-2,2-dimethylpropan-1-ol (479 mg, 4.06 mmol, 2.02 equiv) and methyl 4-bromo-3-hydroxy-2-oxo-2H-pyran-6-carboxylate (product of Example 1, Part A, Step 4) (500 mg, 2.01 mmol, 1.00 equiv) in THF (5 mL) was added triphenylphosphine (PPh₃) (800 mg, 3.05 mmol, 1.52 equiv) at room temperature. To the above mixture was added DBAD (70) mg, 3.04 mmol, 1.51 equiv) at 0° C. The resulting mixture was stirred for additional 1 h at 60° C. then poured into water and extracted with EtOAc (3×100 mL). The combined organic layers were washed with water (3×100 mL) and brine (3×100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (85:15), to afford methyl 4-bromo-3-(3-methoxy-2,2-dimethylpropoxy)-2-oxo-2H-pyran-6-carboxylate (544 mg, 77% yield). LCMS (ES, m/z)=349 [M+1]+. ¹H NMR (400 MHz, DMSO-d6) δ 7.40 (s, 1H), 4.06 (s, 2H), 3.85 (s, 3H), 3.24 (s, 3H), 3.20 (s, 2H), 0.97 (s, 6H).

Step 3: Into a solution of methyl 4-bromo-3-(3-methoxy-2,2-dimethylpropoxy)-2-oxo-2H-pyran-6-carboxylate (540 mg, 1.55 mmol, 1.00 equiv) was added HCl (6M) in water (10 mL) at room temperature. The resulting mixture was stirred overnight at 80° C. then concentrated under reduced pressure. The residue was purified by C18 reverse phase flash chromatography (acetonitrile (MeCN) in water, 0% to 100% gradient in 10 min (H₂O:MeCN=1:1)) to afford 4-bromo-3-(3-methoxy-2,2-dimethylpropoxy)-2-oxo-2H-pyran-6-carboxylic acid (370 mg, 71% yield). LCMS (ES, m/z)=335 [M+1]+.

Step 4: To a stirred solution of 4-bromo-3-(3-methoxy-2,2-dimethylpropoxy)-2-oxo-2H-pyran-6-carboxylic acid (310 mg, 0.925 mmol, 1.00 equiv) and 5-(5-methyl-1H-pyrazol-1-yl)-1,3,4-thiadiazol-2-amine (product of Example 1, Part B, Step 4) (260 mg, 1.44 mmol, 1.55 equiv) in N,N-dimethylformamide (DMF) (3 mL) was added 1-((dimethylamino)(dimethyliminio)methyl)-1H-[1,2,3]triazolo[4,5-b]pyridine 3-oxide hexafluorophosphate(V) (HATU) (530 mg, 1.39 mmol, 1.51 equiv) and diisopropylethylamine (DIEA) (180 mg, 1.39 mmol, 1.51 equiv) at room temperature. The resulting mixture was stirred for 2 h at room temperature. The resulting solids were collected by filtration and washed with water (5×5 mL) to provide 4-bromo-3-(3-methoxy-2,2-dimethylpropoxy)-N-(5-(5-methyl-1H-pyrazol-1-yl)-1,3,4-thiadiazol-2-yl)-2-oxo-2H-pyran-6-carboxamide (310 mg, 67% yield). LCMS (ES, m/z)=498 [M+1]+.

Step 5: 4-(Bicyclo[1.1.1]pentan-1-ylamino)-3-(3-methoxy-2,2-dimethylpropoxy)-N-(5-(5-methyl-JH-pyrazol-1-yl)-1,3,4-thiadiazol-2-yl)-2-oxo-2H-pyran-6-carboxamide (Compound 149) was prepared according to Example 1, Part C, Step 2 using 4-bromo-3-(3-methoxy-2,2-dimethylpropoxy)-N-(5-(5-methyl-JH-pyrazol-1-yl)-1,3,4-thiadiazol-2-yl)-2-oxo-2H-pyran-6-carboxamide as “halo-pyrone reagent” and bicyclo[1.1.1]pentan-1-amine as the “amine reagent”. LCMS (ES, m/z)=501.15 [M+1]+. ¹H NMR (400 MHz, DMSO-d6) δ 7.79 (s, 1H), 7.33 (s, 1H), 6.71 (s, 1H), 6.45 (s, 1H), 3.67 (s, 2H), 3.32 (s, 3H), 3.26 (s, 2H), 2.68 (s, 3H), 2.56 (s, 1H), 2.20 (s, 6H), 0.94 (s, 6H).

### Example 37: (R)-3-methoxy-4-(3-methoxy-2-oxopyrrolidin-1-yl)-N-(5-(5-methyl-1H-pyrazol-1-yl)-1,3,4-thiadiazol-2-yl)-2-oxo-2H-pyran-6-carboxamide (Compound 150)

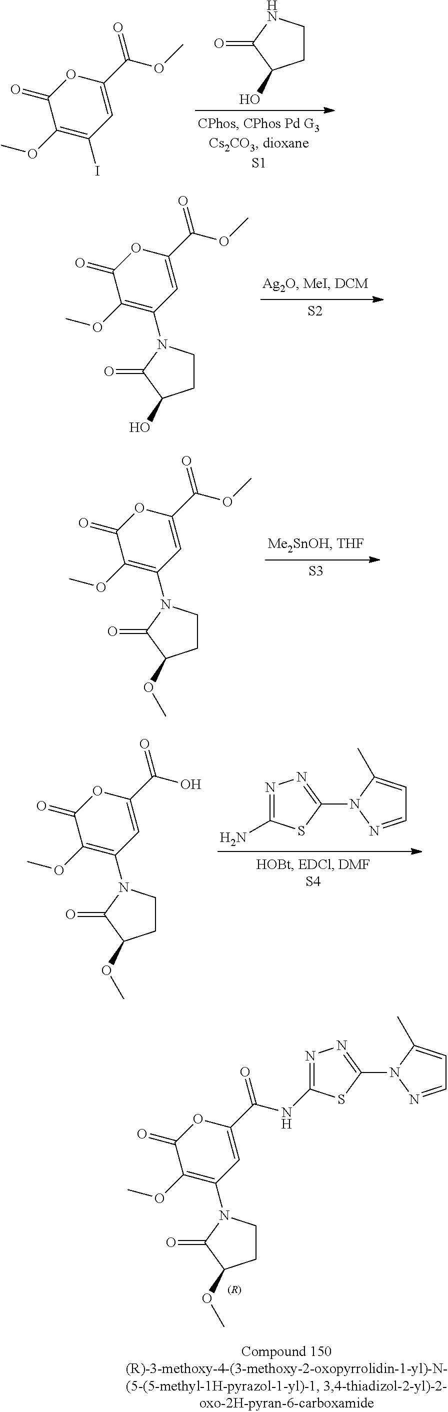

Step 1: To a stirred solution of (3R)-3-hydroxypyrrolidin-2-one (390 mg, 3.86 mmol, 1.99 equiv) and methyl 4-iodo-5-methoxy-6-oxopyran-2-carboxylate (product of Example 6, Step 2) (600 mg, 1.94 mmol, 1.00 equiv) in dioxane (9 mL) was added [(2-dicyclohexylphosphino-2′,6′-bis(N,N-dimethylamino)-1,1′-biphenyl)-2-(2′-amino-1,1′-biphenyl)] palladium(II) methanesulfonate (CPhos Pd G3) (50 mg, 0.061 mmol, 0.19 equiv), 2′-(Dicyclohexylphosphanyl)-N²,N²,N⁶,N⁶-tetramethyl[1,1′-biphenyl]-2,6-diamine (CPhos) (172 mg, 0.394 mmol, 0.20 equiv) and cesium carbonate (1880 mg, 5.77 mmol, 2.98 equiv) at room temperature under N₂ (nitrogen gas). The resulting mixture was stirred for 1 h at 100° C. under N₂ (nitrogen gas) then purified directly by silica gel column chromatography, eluted with petroleum ether/ethyl acetate (PE/EtOAc) (50%-100% yield), to afford methyl 4-[(3R)-3-hydroxy-2-oxopyrrolidin-1-yl]-5-methoxy-6-oxopyran-2-carboxylate (90 mg, 13% yield). LCMS (ES, m/z)=284[M+1]+.

Step 2: To a stirred solution of methyl 4-[(3R)-3-hydroxy-2-oxopyrrolidin-1-yl]-5-methoxy-6-oxopyran-2-carboxylate (50 mg, 0.18 mmol, 1.0 equiv) in dichloromethane (DCM) (0.6 mL) was added Ag₂O (50 mg, 0.22 mmol, 1.22 equiv) and MeI (0.6 mL) at room temperature. The resulting mixture was stirred for 2 h at 50° C. The resulting mixture was then filtered and the filter cake was washed with dichloromethane (DCM) (3×10 mL). The filtrate was concentrated under reduced pressure to provide methyl 5-methoxy-4-[(3R)-3-methoxy-2-oxopyrrolidin-1-yl]-6-oxopyran-2-carboxylate (40 mg, 61% yield). LCMS (ES, m/z)=298 [M+1]+.

Step 3: To a stirred solution of methyl 5-methoxy-4-[(3R)-3-methoxy-2-oxopyrrolidin-1-yl]-6-oxopyran-2-carboxylate (40 mg, 0.14 mmol, 1.0 equiv) in tetrahydrofuran (THF) (1 mL) was added trimethyltin hydroxide (37 mg, 0.20 mmol, 1.52 equiv) at room temperature. The resulting mixture was stirred overnight at room temperature then concentrated under reduced pressure to provide 5-methoxy-4-[(3R)-3-methoxy-2-oxopyrrolidin-1-yl]-6-oxopyran-2-carboxylic acid (30 mg) which was used as is without further purification. LCMS (ES, m/z): 284[M+H]⁺.

Step 4: To a stirred solution of 5-methoxy-4-[(3R)-3-methoxy-2-oxopyrrolidin-1-yl]-6-oxopyran-2-carboxylic acid (“amino-pyrone reagent”) (80 mg, 0.28 mmol, 1.0 equiv) in N,N-dimethylformamide (DMF) (2 mL) was added hydroxybenzotriazole (HOBT) (60 mg, 0.44 mmol, 1.57 equiv), 1-ethyl-3-(3-dimethylaminopropyl)carbodiimide (EDCI) (108 mg, 0.56 mmol, 1.99 equiv) and 5-(5-methylpyrazol-1-yl)-1,3,4-thiadiazol-2-amine (product of Example 1, Part B, Step 4; “ADT amine reagent”) (50 mg, 0.28 mmol, 0.98 equiv) at room temperature. The resulting mixture was stirred for 2 h at room temperature then purified directly by C18 reverse phase flash chromatography (acetonitrile (MeCN) in water (0.1% trifluoroacetic acid (TFA)), 20% to 40% gradient in 10 min; detector, UV 254 nm) to provide crude product which was further purified by Chiral-Prep-HPLC (SHIMADZU: Column, Xselect CSH C18 OBD Column 30*150 mm 5 um; mobile phase, Water (0.05% TFA) and MeCN (29% MeCN up to 39% in 10 min)) to provide (R)-3-methoxy-4-(3-methoxy-2-oxopyrrolidin-1-yl)-N-(5-(5-methyl-1H-pyrazol-1-yl)-1,3,4-thiadiazol-2-yl)-2-oxo-2H-pyran-6-carboxamide (Compound 150) (7.4 mg, 5.8% yield). LCMS (ES, m/z): 447.1 [M+1]+. ¹H NMR (400 MHz, DMSO-d₆) δ 7.89-7.70 (m, 2H), 6.44 (d, J=1.2 Hz, 1H), 4.18 (t, J=8.0 Hz, 1H), 3.92 (s, 3H), 3.85-3.74 (m, 2H), 3.48 (s, 3H), 2.67 (s, 3H), 2.56-2.51 (m, 1H), 2.03-1.94 (m, 1H).

### Example 38: 3-methoxy-4-((1-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 152)

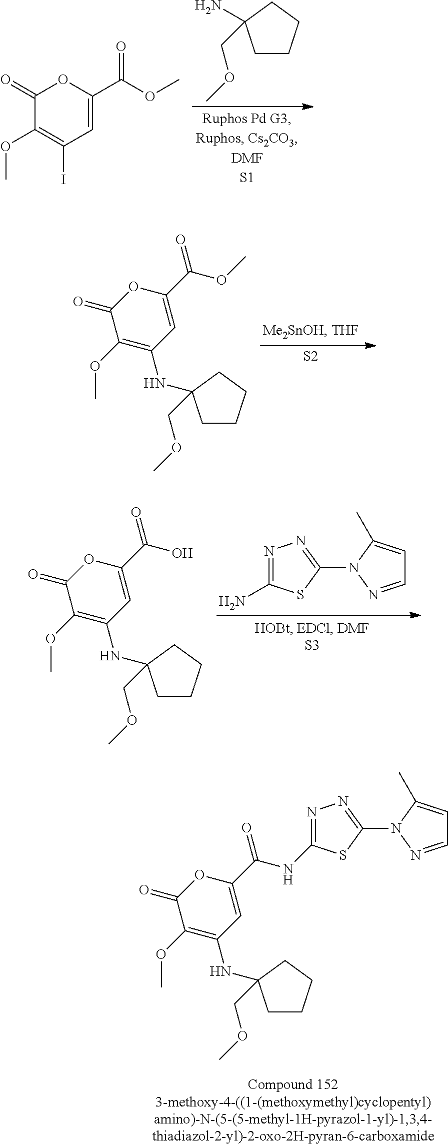

Step 1: Methyl 3-methoxy-4-((1-(methoxymethyl)cyclopentyl)amino)-2-oxo-2H-pyran-6-carboxylate was prepared according to Example 6, Step 5 using methyl 4-iodo-5-methoxy-6-oxopyran-2-carboxylate (product of Example 6, Step 2) and 1-(methoxymethyl)cyclopentan-1-amine. LCMS (ES, m/z)=312 [M+1]+.

Step 2-3: 3-Methoxy-4-((1-(methoxymethyl)cyclopentyl)amino)-N-(5-(5-methyl-1H-pyrazol-1-yl)-1,3,4-thiadiazol-2-yl)-2-oxo-2H-pyran-6-carboxamide (Compound 152) was prepared according to Example 37, Steps 3-4 using methyl 3-methoxy-4-((1-(methoxymethyl)cyclopentyl)amino)-2-oxo-2H-pyran-6-carboxylate in place of methyl 5-methoxy-4-[(3R)-3-methoxy-2-oxopyrrolidin-1-yl]-6-oxopyran-2-carboxylate to provide 3-methoxy-4-((1-(methoxymethyl)cyclopentyl)amino)-2-oxo-2H-pyran-6-carboxylic acid as the “amino-pyrone reagent”, followed by coupling with 5-(5-methyl-1H-pyrazol-1-yl)-1,3,4-thiadiazol-2-amine (product of Example 1, Part B, Step 4) as the “ADT amine reagent”. LCMS (ES, m/z)=461.1 [M+1]+. ¹H NMR (300 MHz, DMSO-d6) δ 7.79 (d, J=1.6 Hz, 1H), 7.27 (s, 1H), 6.45 (d, J=1.6 Hz, 1H), 6.17 (s, 1H), 3.71 (s, 3H), 3.46 (s, 2H), 3.30 (s, 3H), 2.68 (s, 3H), 1.96-1.91 (m, 4H), 1.74-1.63 (m, 4H).

### Example 39: 3-methoxy-4-(((cis)-2-methoxycyclopentyl)(methyl)amino)-N-(5-(5-methyl-1H-pyrazol-1-yl)-1,3,4-thiadiazol-2-yl)-2-oxo-2H-pyran-6-carboxamide (Compound 153, Rac-153), 3-methoxy-4-(((1S,2R)-2-methoxycyclopentyl)(methyl)amino)-N-(5-(5-methyl-1H-pyrazol-1-yl)-1,3,4-thiadiazol-2-yl)-2-oxo-2H-pyran-6-carboxamide (Compound 153a*) and 3-methoxy-4-(((1R,2S)-2-methoxycyclopentyl)(methyl)amino)-N-(5-(5-methyl-1H-pyrazol-1-yl)-1,3,4-thiadiazol-2-yl)-2-oxo-2H-pyran-6-carboxamide (Compound 153b*)

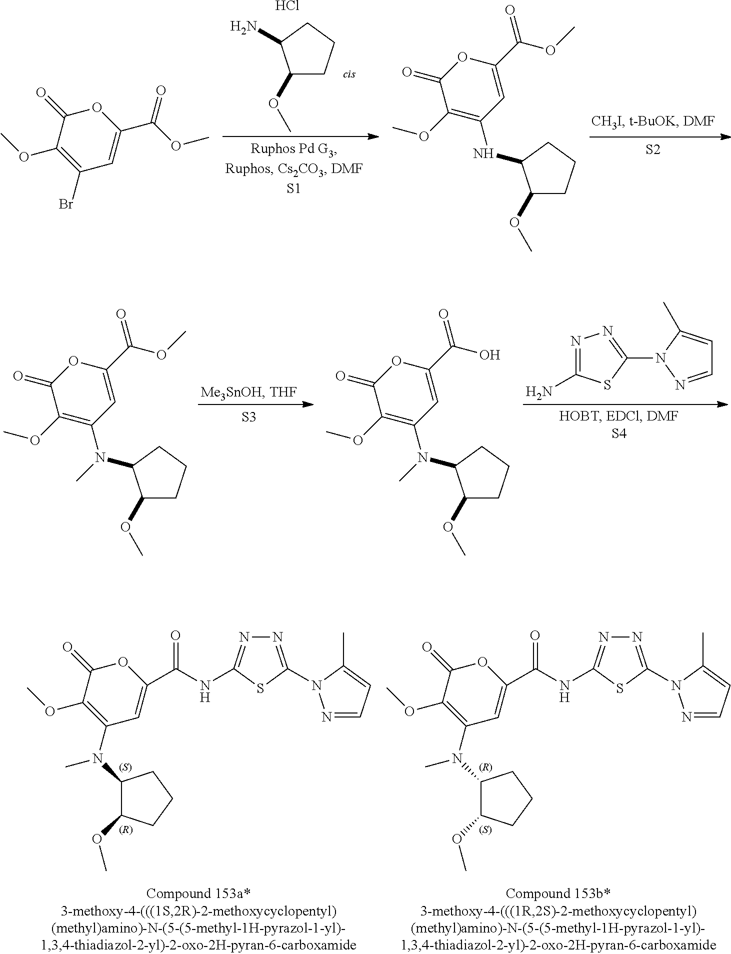

Step 1: Rac-5-methoxy-4-{[cis-2-methoxycyclopentyl]amino}-6-oxopyran-2-carboxylate was prepared according to Example 6 Step 5 using methyl 4-bromo-3-methoxy-2-oxo-2H-pyran-6-carboxylate and (1,2-cis)-2-methoxycyclopentan-1-amine hydrochloride. LCMS (ES, m/z)=298 [M+1]+.

Step 2: To a stirred solution of rac-methyl 5-methoxy-4-{[cis-2-methoxycyclopentyl]amino}-6-oxopyran-2-carboxylate (81 mg, 0.27 mmol, 1.0 equiv) in dimethylformamide (DMF) (5 mL) were added methyl iodide (360 mg, 2.54 mmol, 9.31 equiv) and tert-butoxypotassium (122 mg, 1.09 mmol, 3.99 equiv) at 0° C. The resulting mixture was stirred overnight at room temperature. The mixture was then diluted with water (100 mL) and extracted with ethyl acetate (EtOAc) (3×100 mL). The combined organic layers were washed with brine (50 mL), and dried over anhydrous Na₂SO₄. After filtration, the filtrate was concentrated under reduced pressure and purified by silica gel column chromatography, eluted with petroleum ether/ethyl acetate (PE/EtOAc) (1:1), to afford rac-methyl 5-methoxy-4-{[cis-2-methoxycyclopentyl](methyl)amino}-6-oxopyran-2-carboxylate (63 mg, 66% yield). LCMS (ES, m/z)=312 [M+1]+.

Steps 3-4: 3-Methoxy-4-(((1S,2R)-2-methoxycyclopentyl)(methyl)amino)-N-(5-(5-methyl-1H-pyrazol-1-yl)-1,3,4-thiadiazol-2-yl)-2-oxo-2H-pyran-6-carboxamide (Compound 153a*) and 3-methoxy-4-(((1R,2S)-2-methoxycyclopentyl)(methyl)amino)-N-(5-(5-methyl-1H-pyrazol-1-yl)-1,3,4-thiadiazol-2-yl)-2-oxo-2H-pyran-6-carboxamide (Compound 153b*) were prepared as a racemic mixture from rac-methyl 5-methoxy-4-{[cis-2-methoxycyclopentyl](methyl)amino}-6-oxopyran-2-carboxylate according to Example 37 steps 3-4 using rac-methyl 5-methoxy-4-{[cis-2-methoxycyclopentyl](methyl)amino}-6-oxopyran-2-carboxylate in place of methyl 5-methoxy-4-[(3R)-3-methoxy-2-oxopyrrolidin-1-yl]-6-oxopyran-2-carboxylate to provide Rac-3-methoxy-4-(((1,2-cis)-2-methoxycyclopentyl)(methyl)amino)-2-oxo-2H-pyran-6-carboxylic acid as the “amino-pyrone reagent”, followed by coupling with 5-(5-methyl-1H-pyrazol-1-yl)-1,3,4-thiadiazol-2-amine (product of Example 1, Part B, Step 4) as the “ADT amine reagent”. LCMS (ES, m/z)=461.1 [M+1]+. ¹H NMR (400 MHz, DMSO-d6) δ 13.40 (br, 1H), 7.78 (s, 1H), 7.33 (s, 1H), 6.44 (s, 1H), 4.26-4.20 (m, 1H), 3.87-3.79 (m, 1H), 3.68 (s, 3H), 3.22 (s, 3H), 3.10 (s, 3H), 2.67 (s, 3H), 2.04-1.97 (m, 1H), 1.79-1.65 (m, 4H), 1.53-1.47 (m, 1H).

### Example 40: N-(5-(3,5-dichloroisothiazol-4-yl)-1,3,4-thiadiazol-2-yl)-3-methoxy-4-((2-methoxyethyl)amino)-2-oxo-2H-pyran-6-carboxamide (Compound 156)

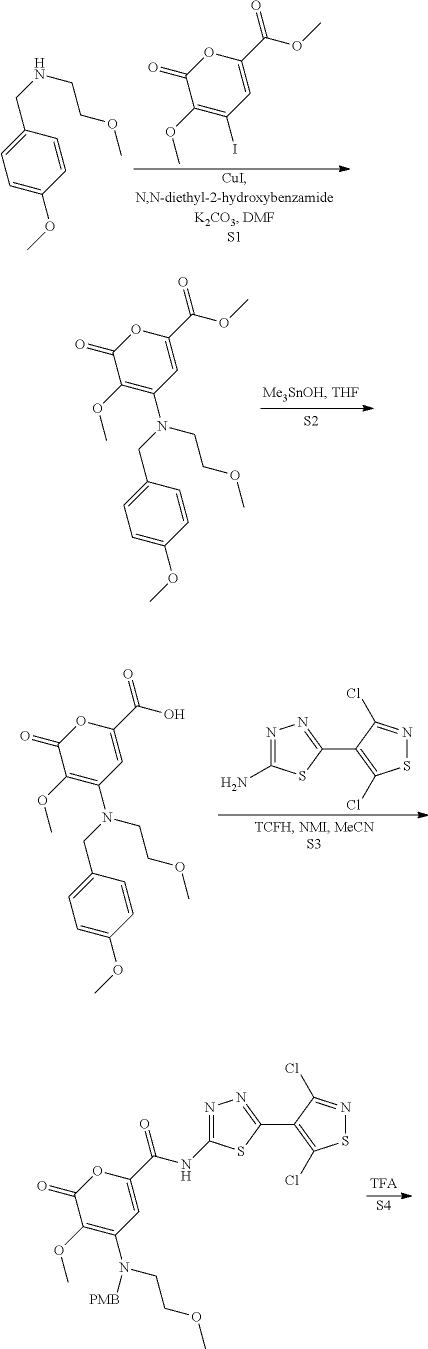

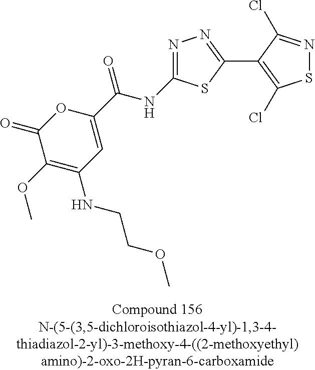 [SMILES: COCCNc1cc(C(=O)Nc2nnc(-c3c(Cl)nsc3Cl)s2)oc(=O)c1OC]

Step 1: To a mixture of (2-methoxyethyl) [(4-methoxyphenyl) methyl] amine (1000 mg, 5.12 mmol, 1.00 equiv) and methyl 4-iodo-5-methoxy-6-oxopyran-2-carboxylate (product of Example 6, Step 2) (1000 mg, 3.22 mmol, 0.63 equiv) in N,N-dimethylformamide (DMF) (10 mL) was added CuI (205 mg, 1.08 mmol, 0.21 equiv), N,N-diethyl-2-hydroxybenzamide (198 mg, 1.02 mmol, 0.20 equiv) and K₂CO₃ (1000 mg, 7.24 mmol, 1.41 equiv). The resulting mixture was stirred for 1 h at 100° C. under N₂ (nitrogen gas) then diluted with water (50 mL). The mixture was extracted with ethyl acetate (EtOAc) (3×150 mL). The combined organic layers were washed with water (3×10 mL), and dried over anhydrous Na₂SO₄. After filtration, the filtrate was concentrated under reduced pressure and purified by silica gel column chromatography, eluted with petroleum ether/ethyl acetate (PE/EtOAc) (1:2), to afford methyl 5-methoxy-4-[(2-methoxyethyl) [(4-methoxy phenyl) methyl] amino]-6-oxopyran-2-carboxylate (600 mg, 31% yield). LCMS (ES, m/z)=378.0 [M+1]+.

Step 2: To a mixture of methyl 5-methoxy-4-[(2-methoxyethyl) [(4-methoxyphenyl) methyl] amino]-6-oxopyran-2-carboxylate (190 mg, 0.503 mmol, 1.00 equiv) in tetrahydrofuran (THF) (2 mL) was added trimethyltin hydroxide (137 mg, 0.758 mmol, 1.50 equiv). The resulting mixture was stirred for 2 h at room temperature then concentrated under reduced pressure. The crude product 5-methoxy-4-[(2-methoxyethyl) [(4-methoxyphenyl) methyl] amino]-6-oxopyran-2-carboxylic acid was used in the next step directly without further purification. LCMS (ES, m/z)=364.0 [M+1]+.

Step 3: To a mixture of 5-methoxy-4-[(2-methoxyethyl) [(4-methoxyphenyl) methyl] amino]-6-oxopyran-2-carboxylic acid (“amino-pyrone reagent”) (190 mg, 0.523 mmol, 1.00 equiv) and 5-(3,5-dichloro-1,2-thiazol-4-yl)-1,3,4-thiadiazol-2-amine (133 mg, 0.525 mmol, 1.00 equiv) as the “ADT amine reagent” (which may be synthesized following Example 29, Step 1, but using 3,5-dichloroisothiazole-4-carboxylic acid instead of 3-chloro-1,2-thiazole-4-carboxylic acid) in acetonitrile (MeCN) (3 mL) was added chloro-N,N,N′,N′-tetramethylformamidinium hexafluorophosphate (TCFH) (300 mg, 1.07 mmol, 2.04 equiv) and N-methylimidazole (NMI) (130 mg, 1.58 mmol, 3.03 equiv). The resulting mixture was stirred for 1 h at room temperature then diluted with water (20 mL). The mixture was extracted with EtOAc (5×20 mL). The combined organic layers were washed with brine (10 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (1:1), to afford N-[5-(3,5-dichloro-1,2-thiazol-4-yl)-1,3,4-thiadiazol-2-yl]-5-methoxy-4-[(2-methoxyethyl) 1(4-methoxyphenyl) methyl] aminol-6-oxopyran-2-carboxamide (70 mg, 22% yield). LCMS (ES, m/z)=598.0 [M+1]+. PMB=4-methoxybenzyl.

Step 4: Into a 25 mL round-bottom flask was added N-[5-(3,5-dichloro-1,2-thiazol-4-yl)-1,3,4-thiadiazol-2-yl]-5-methoxy-4-[(2-methoxyethyl) [(4-methoxyphenyl) methyl] amino]-6-oxopyran-2-carboxamide (50 mg, 0.084 mmol, 1.00 equiv) and trifluoroacetic acid (1 mL). The resulting mixture was stirred for 1 h at room temperature then concentrated under reduced pressure. The residue was then purified by C18 reverse phase flash chromatography (acetonitrile (MeCN) in water, 10% to 50% gradient in 20 min; detector, UV 254 nm) to provide N-(5-(3,5-dichloroisothiazol-4-yl)-1,3,4-thiadiazol-2-yl)-3-methoxy-4-((2-methoxyethyl)amino)-2-oxo-2H-pyran-6-carboxamide (Compound 156) (11 mg, 27% yield). LCMS (ES, m/z)=477.95 [M+1]+. ¹H NMR (400 MHz, DMSO-d6) δ 7.42 (s, 1H), 7.10 (s, 1H), 3.70 (s, 3H), 3.49 (t, J=2.4 Hz, 4H), 3.29 (s, 3H).

### Example 41: 4-((2-hydroxyethyl)(2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 157), 4-((2-(benzyloxy)ethyl)(2-methoxyethyl)amino)-3-methoxy-N-(5-(5-methyl-1H-pyrazol-1-yl)-1,3,4-thiadiazol-2-yl)-2-oxo-2H-pyran-6-carboxamide (Compound 157-OBn), 4-(bis(2-hydroxyethyl)amino)-3-methoxy-N-(5-(5-methyl-1H-pyrazol-1-yl)-1,3,4-thiadiazol-2-yl)-2-oxo-2H-pyran-6-carboxamide (Compound 158)

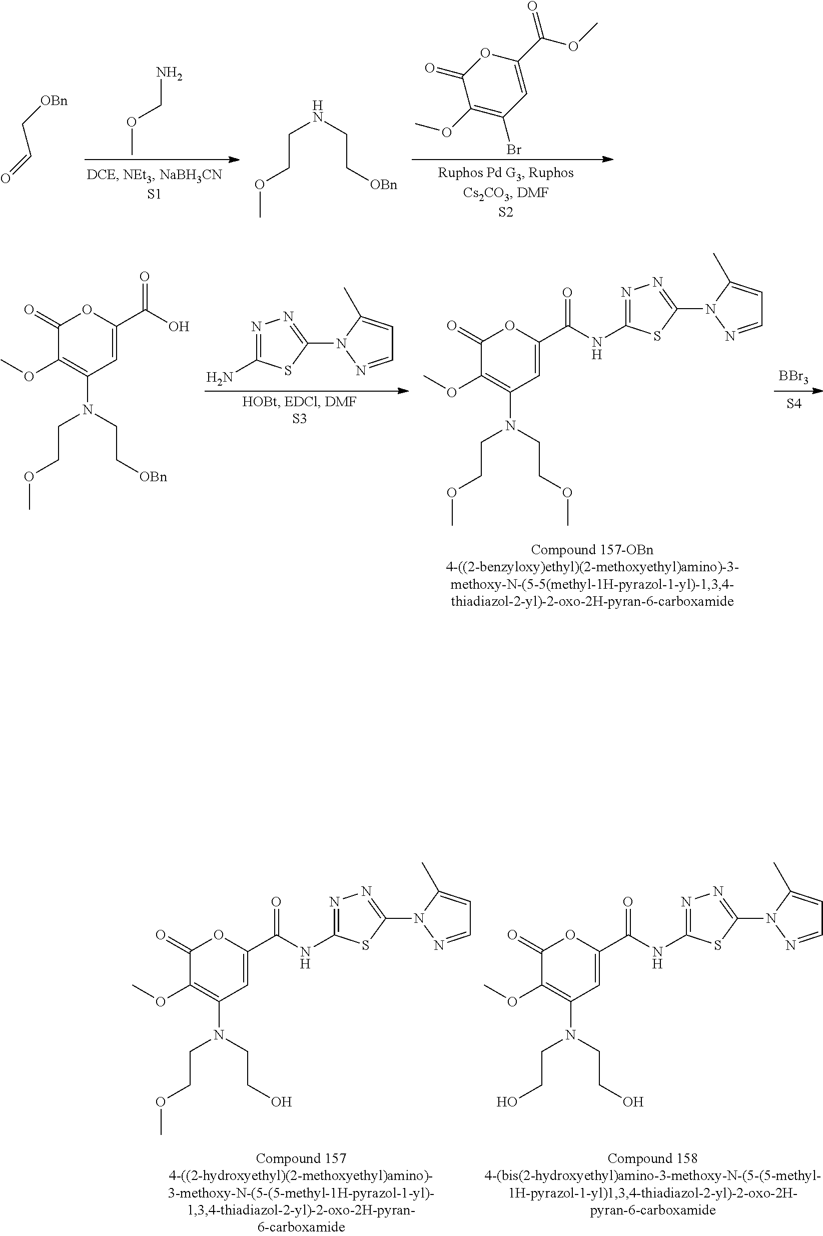

Step 1: To a stirred mixture of 2-(benzyloxy)acetaldehyde (1.0 g, 6.66 mmol, 1.0 equiv) and 2-methoxyethan-1-amine (666 mg, 8.87 mmol, 1.33 equiv) in dichloroethane (DCE) (50 mL) was added triethylamine (NEt₃) (1.0 g, 9.9 mmol, 1.5 equiv) and sodium triacetoxyborohydride (NaBH(OAc)₃) (2.46 g, 11.6 mmol, 1.74 equiv). The resulting mixture was stirred for 1 h at room temperature then quenched with NaHCO₃ at room temperature. The mixture was extracted with dichloromethane (DCM). The organic layer was washed with water, and dried over anhydrous Na₂SO₄. After filtration, the filtrate was concentrated under reduced pressure and the residue was purified by silica gel column chromatography, eluted with petroleum ether/ethyl acetate (PE/EtOAc) (1:1), to afford [2-(benzyloxy)ethyl](2-methoxyethyl)amine (700 mg, 46% yield). LCMS (ES, m/z)=210 [M+1]+.

Step 2: To a stirred mixture of [2-(benzyloxy)ethyl](2-methoxyethyl)amine (430 mg, 2.05 mmol, 1.00 equiv) and methyl 4-bromo-5-methoxy-6-oxopyran-2-carboxylate (product of Example 1, Part A, Step 3) (443 mg, 1.68 mmol, 0.82 equiv) in N,N-dimethylformamide (DMF) (5 mL) was added (2-dicyclohex)ylphosphino-2′,6′-diisopropoxy-1,1′-biphenyl)[2-(2′-amino-1,1′-biphenyl)]palladium(II) methanesulfonate (RuPhos Palladacycle Gen3) (140 mg, 0.167 mmol, 0.08 equiv), 2-Dicyclohexylphosphino-2′,6′-diisopropoxybiphenyl (RuPhos) (157 mg, 0.336 mmol, 0.16 equiv) and Cesium carbonate (1196 mg, 3.67 mmol, 1.79 equiv). The resulting mixture was stirred for 1 h at 110° C. then directly purified by C18 reverse phase flash chromatography (acetonitrile (MeCN) in water (5 mmol/L NH₄HCO₃), 0% to 100% gradient in 30 min; detector, UV 254 nm) to provide 4-{[2-(benzyloxy)ethyl](2-methoxyethyl)amino}-5-methoxy-6-oxopyran-2-carboxylic acid (190 mg, 24% yield). LCMS (ES, m/z)=378 [M+1]+.

Step 3: To a stirred mixture of 4-{[2-(benzyloxy)ethyl](2-methoxyethyl)amino}-5-methoxy-6-oxopyran-2-carboxylic acid (“amino-pyrone reagent”) (180 mg, 0.477 mmol, 1.00 equiv) and 5-(5-methylpyrazol-1-yl)-1,3,4-thiadiazol-2-amine (product of Example 1, Part B, Step 4; “ADT amine reagent”) (83 mg, 0.46 mmol, 0.96 equiv) in N,N-dimethylformamide (DMF) (2 mL) was added hydroxybenzotriazole (HOBT) (124 mg, 0.918 mmol, 1.92 equiv) and 1-ethyl-3-(3-dimethylaminopropyl)carbodiimide (EDCI) (264 mg, 1.38 mmol, 2.89 equiv). The resulting mixture was stirred for 1 h at room temperature under air atmosphere then extracted with ethyl acetate (EtOAc). The organic layer was washed with brine, and dried over anhydrous Na₂SO₄. After filtration, the filtrate was concentrated under reduced pressure and purified by silica gel column chromatography, eluted with petroleum ether/ethyl acetate (PE/EtOAc) (1:1), to afford 4-{[2-(benzyloxy)ethyl](2-methoxyethyl)amino}-5-methoxy-N-[5-(5-methylpyrazol-1-yl)-1,3,4-thiadiazol-2-yl]-6-oxopyran-2-carboxamide (also referred to herein as 4-((2-(benzyloxy)ethyl)(2-methoxyethyl)amino)-3-methoxy-N-(5-(5-methyl-1H-pyrazol-1-yl)-1,3,4-thiadiazol-2-yl)-2-oxo-2H-pyran-6-carboxamide; Compound 157-OBn) (75 mg, 29% yield). LCMS (ES, m/z)=541 [M+1]+.

Step 4: To a stirred mixture of 4-{[2-(benzyloxy)ethyl](2-methoxyethyl)amino}-5-methoxy-N-[5-(5-methylpyrazol-1-yl)-1,3,4-thiadiazol-2-yl]-6-oxopyran-2-carboxamide (Compound 157-OBn) (50 mg, 0.092 mmol, 1.00 equiv) in dichloromethane (DCM) (0.5 mL) was added BBr₃ (0.6 ml, 0.6 mmol, 6.47 equiv) in DCM (1 M) at 0° C. The resulting mixture was stirred for 40 min at room temperature then concentrated under reduced pressure. The crude product was purified by Prep-HPLC (XBridge Shield RP18 OBD Column, 30*150 mm, 5 μm; Mobile Phase A: water (10 mmol/L NH₄HCO₃), Mobile Phase B: acetonitrile (MeCN); Flow rate: 60 mL/min; Gradient: 10% B to 20/u B in 10 min, 20% B; Wave Length: 254 nm; RT1 (min); 8.8, RT2 (min): 9.8) to afford 4-((2-hydroxyethyl)(2-methoxyethyl)amino)-3-methoxy-N-(5-(5-methyl-1H-pyrazol-1-yl)-1,3,4-thiadiazol-2-yl)-2-oxo-2H-pyran-6-carboxamide (2.9 mg, 6.5% yield) (Compound 157), first eluting peak, LCMS (ES, m/z)=451.15 [M+1]+. ¹H NMR (400 MHz, DMSO-d6) δ7.62 (s, 1H), 7.06 (d, J=1.2 Hz, 1H), 6.31 (d, J=0.8 Hz, 1H), 4.86 (t, J=4.8 Hz, 1H), 3.59-3.52 (m, 11H), 3.29 (s, 3H), 2.60 (s, 3H); and 4-(bis(2-hydroxyethyl)amino)-3-methoxy-N-(5-(5-methyl-1H-pyrazol-1-yl)-1,3,4-thiadiazol-2-yl)-2-oxo-2H-pyran-6-carboxamide (4.3 mg, 9.7% yield) (Compound 158), second eluting peak, LCMS (ES, m/z)=437.1 [M+1]+, ¹H NMR (400 MHz, DMSO-d6) δ 7.62 (s, 1H), 7.07 (d, J=1.2 Hz, 1H), 6.30 (d, J=0.8 Hz 1H), 4.94-4.82 (m, 2H), 3.62-3.60 (m, 4H), 3.47-3.48 (m, 7H), 2.59 (s, 3H).

### Example 42: 4-(((cis)-2-hydroxycyclopentyl)(methyl)amino)-3-methoxy-N-(5-(5-methyl-1H-pyrazol-1-yl)-1,3,4-thiadiazol-2-yl)-2-oxo-2H-pyran-6-carboxamide (Compound 159, Rac-159), 4-(((1S,2R)-2-hydroxycyclopentyl)(methyl)amino)-3-methoxy-N-(5-(5-methyl-1H-pyrazol-1-yl)-1,3,4-thiadiazol-2-yl)-2-oxo-2H-pyran-6-carboxamide (Compound 159a*) and 4-(((1R,2S)-2-hydroxycyclopentyl)(methyl)amino)-3-methoxy-N-(5-(5-methyl-1H-pyrazol-1-yl)-1,3,4-thiadiazol-2-yl)-2-oxo-2H-pyran-6-carboxamide (Compound 159b*)

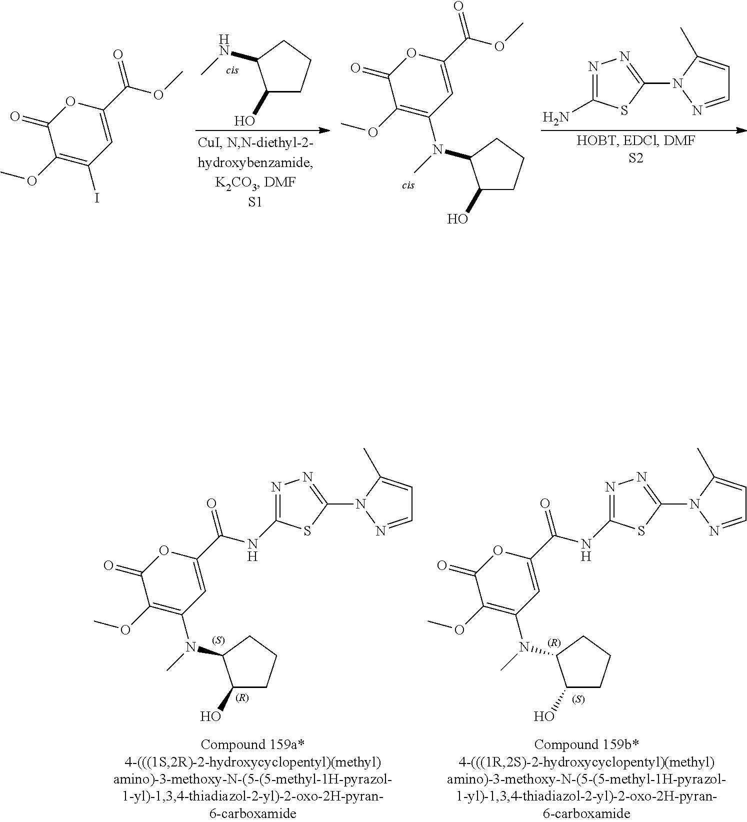

Step 1: 4-((cis-2-hydroxycyclopentyl)(methyl)amino)-3-methoxy-2-oxo-2H-pyran-6-carboxylic acid was prepared using cis-2-(methylamino)cyclopentan-1-ol and methyl 4-iodo-3-methoxy-2-oxo-2H-pyran-6-carboxylate (also referred to herein as methyl 4-iodo-5-methoxy-6-oxopyran-2-carboxylate; product of Example 6, Step 2) according to the procedure outlined in Example 40, Step 1. LCMS (ES, m/z)=284.0 [M+1]+.

Step 2: Into a solution of 4-(cis-2-hydroxycyclopentyl)(methyl)amino)-3-methoxy-2-oxo-2H-pyran-6-carboxylic acid (40 mg, 0.14 mmol, 1.00 equiv) in N,N-dimethylformamide (DMF) (2 mL) was added hydroxybenzotriazole (HOBT) (29 mg, 0.215 mmol, 1.52 equiv), 1-ethyl-3-(3-dimethylaminopropyl)carbodiimide (EDCI) (54 mg, 0.282 mmol, 1.99 equiv) and 5-(5-methylpyrazol-1-yl)-1,3,4-thiadiazol-2-amine (product of Example 1, Part B, Step 4) (23 mg, 0.127 mmol, 0.90 equiv) at room temperature. The resulting mixture was stirred for 2 h at room temperature then concentrated under reduced pressure. The residue was purified by Prep-HPLC (SHIMADZU: Column, XBridge Prep OBD C18 Column, 30*150 mm, 5 μm; mobile phase, Water (10 mmol/L NH₄HCO₃) and acetonitrile (MeCN) (19% MeCN up to 27% in 8 min); Detector, UV 254 nm) to provide 4-(((1S,2R)-2-hydroxycyclopentyl)(methyl)amino)-3-methoxy-N-(5-(5-methyl-1H-pyrazol-1-yl)-1,3,4-thiadiazol-2-yl)-2-oxo-2H-pyran-6-carboxamide (Compound 159a*) and 4-(((1R,2S)-2-hydroxycyclopentyl)(methyl)amino)-3-methoxy-N-(5-(5-methyl-1H-pyrazol-1-yl)-1,3,4-thiadiazol-2-yl)-2-oxo-2H-pyran-6-carboxamide (Compound 159b*) as a racemic mixture (14 mg, 23% yield). LCMS (ES, m/z)=447.10 [M+1]+. ¹H NMR (400 MHz, DMSO-d6) δ 7.63 (s, 1H), 7.12 (s, 1H), 6.32 (s, 1H), 4.20-4.18 (s, 1H), 4.06-4.03 (m, 1H), 3.65 (s, 3H), 3.12 (s, 3H), 2.59 (s, 3H), 2.07-1.95 (m, 1H), 1.94-1.71 (m, 3H), 1.51-1.45 (m, 2H).

### Example 43: 3-methoxy-4-(((3S,4S)-4-methoxytetrahydrofuran-3-yl)amino)-N-(5-(S-methyl-1H-pyrazol-1-yl)-1,3,4-thiadiazol-2-yl)-2-oxo-2H-pyran-6-carboxamide (Compound 160)

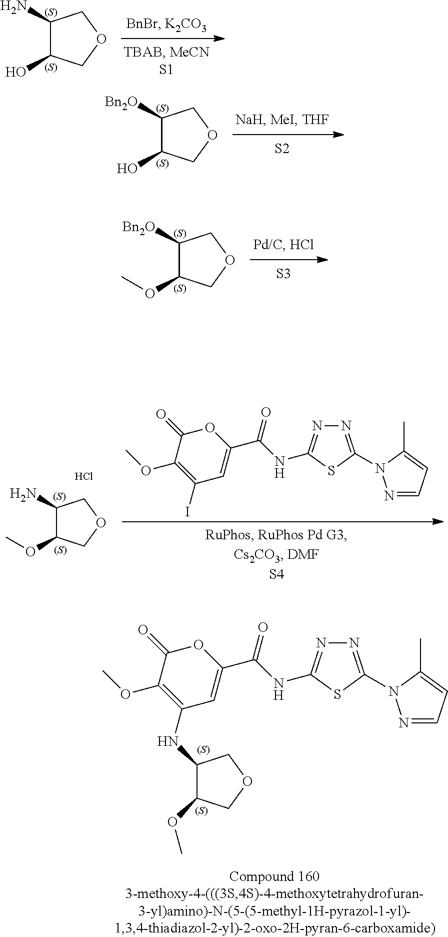

Step 1: To a stirred solution of (3S,4S)-4-aminooxolan-3-ol (300 mg, 2.91 mmol, 1.00 equiv) in acetonitrile (MeCN) (500 mL) was added benzyl bromide (BnBr) (1200 mg, 7.02 mmol, 2.41 equiv). K₂CO₃ (900 mg, 6.51 mmol, 2.24 equiv) and tetra-n-butylammonium bromide (TBAB) (180 mg, 0.56 mmol, 0.19 equiv) at room temperature. The resulting mixture was stirred overnight at room temperature then diluted with water (50 mL). The mixture was then extracted with ethyl acetate (EtOAc) (3×50 mL). The combined organic layers were washed with brine (2×8 mL), and dried over anhydrous Na₂SO₄. After filtration, the filtrate was concentrated under reduced pressure and purified by silica gel column chromatography, eluted with petroleum ether/ethyl acetate (PE/EtOAc) (1:1), to afford (3S,4S)-4-(dibenzylamino)oxolan-3-ol (760 mg, 92% yield). LCMS (ES, m/z)=284 [M+1]+.

Step 2: To a solution of (3S,4S)-4-(dibenzylamino)oxolan-3-ol (760 mg, 2.68 mmol, 1.00 equiv) in tetrahydrofuran (THF) (10 mL) was added 60% NaH in mineral oil (220 mg, 5.50 mmol, 2.05 equiv) at 0° C. The mixture was stirred for 30 min then methyl iodide (MeI) (1530 mg, 10.78 mmol, 4.02 equiv) was added and the mixture was allowed to warm to rt and stirred overnight. The reaction was quenched with water at room temperature and extracted with ethyl acetate (EtOAc) (3×70 mL). The combined organic layers were washed with brine (2×10 mL), and dried over anhydrous Na₂SO₄. After filtration, the filtrate was concentrated under reduced pressure and purified by silica gel column chromatography, eluted with petroleum ether/ethyl acetate (PE/EtOAc) (4:1), to afford (3S,4S)—N,N-dibenzyl-4-methoxyoxolan-3-amine (700 mg, 88% yield). LCMS (ES, m/z)=298 [M+1]+.

Step 3: To a solution of (3S,4S)—N,N-dibenzyl-4-methoxyoxolan-3-amine (350 mg, 1.18 mmol, 1.00 equiv) in tetrahydrofuran (THF) (20 mL) was added Pd/C (150 mg, 1.41 mmol, 1.20 equiv) and 2M HCl (5.5 mL, 11.00 mmol, 9.35 equiv). The mixture was stirred at room temperature for 2 h under H₂ (gas). The resulting mixture was filtered through a Celite pad and concentrated under reduced pressure to provide crude (3S,4S)-4-methoxytetrahydrofuran-3-amine hydrochloride (200 mg) which was used without further purification. LCMS (ES, m/z)=118 [M+1]+.

Step 4: 3-Methoxy-4-(((3S,4S)-4-methoxytetrahydrofuran-3-yl)amino)-N-(5-(5-methyl-1H-pyrazol-1-yl)-1,3,4-thiadiazol-2-yl)-2-oxo-2H-pyran-6-carboxamide (Compound 160) was prepared according to Example 6, Step 5 using 4-iodo-5-methoxy-N-[5-(5-methylpyrazol-1-yl)-1,3,4-thiadiazol-2-yl]-6-oxopyran-2-carboxamide (product of Step 4 of Example 6) as “halo-pyrone reagent” and (3S,4S)-4-methoxytetrahydrofuran-3-amine hydrochloride as the “amine reagent”. LCMS (ES, m/z)=449.1 [M+1]+. ¹H NMR (400 MHz, DMSO-d6) δ 7.73 (d, J=1.6 Hz, 1H), 7.36 (s, 1H), 6.45 (d, J=1.6 Hz, 1H), 6.25 (br, 1H), 4.47-4.41 (m, 1H), 4.03-3.96 (m, 2H), 3.91-3.82 (m, 2H), 3.72 (s, 3H), 3.65-3.62 (m, 1H), 3.33 (s, 3H), 2.66 (s, 3H).

### Example 44: (S)-3-methoxy-4-((1-methoxy-3-phenylpropan-2-yl)amino)-N-(5-(5-methyl-1H-pyrazol-1-yl)-1,3,4-thiadiazol-2-yl)-2-oxo-2H-pyran-6-carboxamide (Compound 165)

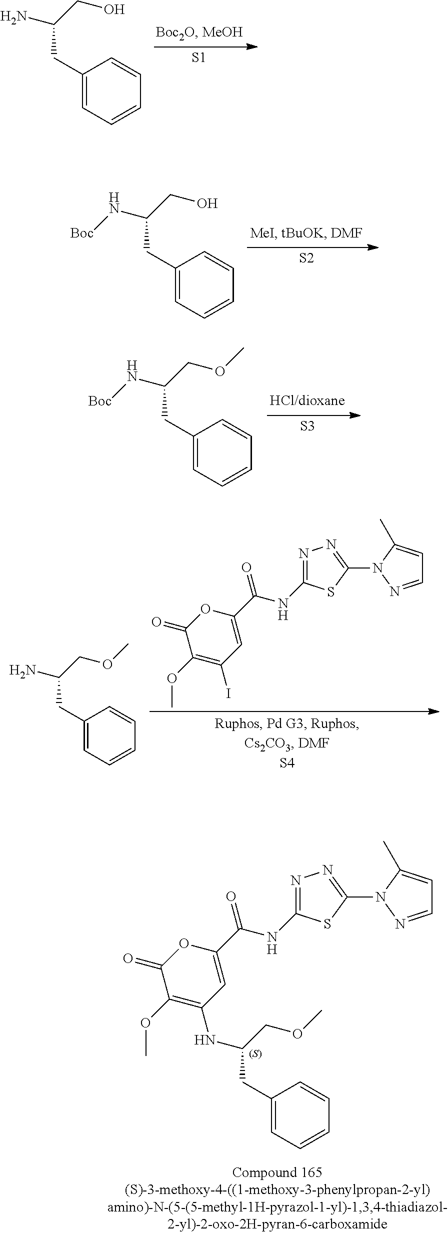

Step 1: To a stirred solution of L-phenylalaninol (1.0 g, 6.61 mmol, 1.0 equiv) in methanol (MeOH) (30 mL) was added di-tert-butyl decarbonate (Boc₂O) (2.90 g, 13.3 mmol, 2.01 equiv) at room temperature. The resulting mixture was stirred overnight at room temperature then concentrated under vacuum. The residue was dissolved with ethyl acetate (EtOAc) (100 mL) and washed with water (2×10 mL), and solids crashed out of the solution. The resulting solids were collected and dried in an oven under reduced pressure. The solids were then purified by silica gel column chromatography, eluted with petroleum ether/ethyl acetate (PE/EtOAc) (5:1), to afford tert-butyl N-[(2S)-1-hydroxy-3-phenylpropan-2-yl]carbamate (1.13 g, 48% yield). LCMS (ES, m/z)=252 [M+1]+.

Step 2: To a stirred solution of tert-butyl N-[(2S)-1-hydroxy-3-phenylpropan-2-yl]carbamate (940 mg, 3.74 mmol, 1.00 equiv) in dimethylformamide (DMF) (19 mL) was added methyl iodide (MeI) (1594 mg, 11.23 mmol, 3.00 equiv) was stirred for 15 min at 0° C. To the above mixture was added tert-butoxypotassium (630 mg, 5.61 mmol, 1.50 equiv) at 0° C. The resulting mixture was stirred for additional 2 h at room temperature. The reaction was quenched with sat. NH₄Cl (aq.) at room temperature. The mixture was then diluted with water (150 mL) and the aqueous layer was extracted with EtOAc (3×150 mL). The combined organic layers were washed with water (3×10 mL), and dried over anhydrous Na₂SO₄, filtered and concentrated. The residue was purified by silica gel column chromatography, eluted with petroleum ether/ethyl acetate (PE/EtOAc) (5:1), to afford tert-butyl N-[(2S)-1-methoxy-3-phenylpropan-2-yl]carbamate (845 mg, 60% yield). LCMS (ES, m/z)=266 [M+1]+.

Step 3: To a stirred solution of tert-butyl N-[(2S)-1-methoxy-3-phenylpropan-2-yl]carbamate (780 mg, 2.94 mmol, 1.00 equiv) in HCl in dioxane (7.8 mL, 4.0 M) at room temperature. The resulting mixture was stirred for 1 h at room temperature. The mixture was then diluted with water (100 mL) and neutralized to pH 8 with saturated NaHCO₃ (aq.). The aqueous layer was extracted with ethyl acetate (EtOAc) (3×150 mL). The combined organic layers were washed with water (3×10 mL), and dried over anhydrous Na₂SO₄, filtered and concentrated. The residue was purified by silica gel column chromatography, eluted with petroleum ether/ethyl acetate (PE/EtOAc) (1:1), to afford (2S)-1-methoxy-3-phenylpropan-2-amine (370 mg, 44% yield). LCMS (ES, m/z)=166 [M+1]+.

Step 4: (S)-3-Methoxy-4-((1-methoxy-3-phenylpropan-2-yl)amino)-N-(5-(5-methyl-1H-pyrazol-1-yl)-1,3,4-thiadiazol-2-yl)-2-oxo-2H-pyran-6-carboxamide (Compound 165) was prepared according to Example 6, Step 5 using 4-iodo-5-methoxy-N-[5-(5-methylpyrazol-1-yl)-1,3,4-thiadiazol-2-yl]-6-oxopyran-2-carboxamide (product of Step 4 of Example 6) as “halo-pyrone reagent” and (2S)-1-methoxy-3-phenylpropan-2-amine as the “amine reagent”. LCMS (ES, m/z)=497.16 [M+1]+. ¹H NMR (400 MHz, DMSO-d6) δ 7.61 (d, J=1.2 Hz, 1H), 7.33-7.26 (m, 4H), 7.18 (dd, J=2.0, 8.8 Hz 1H), 6.90 (s, 1H), 6.47 (s, 1H), 6.31 (s, 1H), 4.03-3.97 (m, 1H), 3.57 (s, 3H), 3.47-3.43 (m, 2H), 3.31 (s, 3H), 2.92-2.88 (m, 2H), 2.60 (s, 3H).

### Example 45: 3-methoxy-4-(((2R,3S)-3-methoxybutan-2-yl)amino)-N-(5-(5-methyl-1H-pyrazol-1-yl)-1,3,4-thiadiazol-2-yl)-2-oxo-2H-pyran-6-carboxamide (Compound 166)

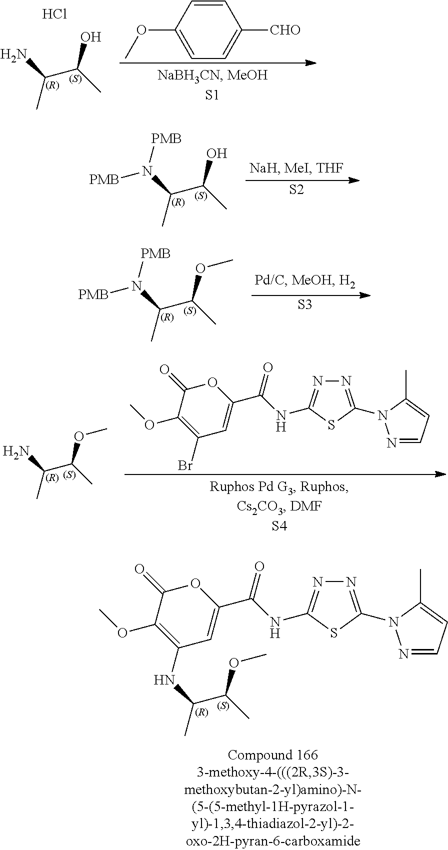

Step 1: To a solution of (2S,3R)-3-aminobutan-2-ol hydrochloride (600 mg, 4.78 mmol, 1.00 equiv) in methanol (MeOH) (10 mL) was added anisaldehyde (1.95 g, 14.3 mmol, 3.00 equiv). The resulting mixture was stirred for 0.5 h at room temperature. To the above mixture was added sodium cyanoborohydride (NaBH₃CN) (200 mg, 3.18 mmol, 2.00 equiv) at room temperature. The resulting mixture was stirred for additional 2 h at room temperature then concentrated under reduced pressure. The residue was then purified by C18 reverse phase flash chromatography (acetonitrile (MeCN) in water (0.1% formic acid (FA)), 10% to 50% gradient in 10 min; detector, UV 254 nm) to provide (2S,3R)-3-{bis[(4-methoxyphenyl)methyl]amino}butan-2-ol (720 mg, 40% yield). LCMS (ES, m/z)=330 [M+1]+. PMB=paramethoxybenzyl.

Step 2: To a solution of (2S,3R)-3-{bis[(4-methoxyphenyl)methyl]amino}butan-2-ol (1.10 g, 3.34 mmol, 1.00 equiv) in tetrahydrofuran (THF) (30 mL) was added NaH (60% in mineral oil, 262 mg, 6.56 mmol, 2.00 equiv) in portions at 0° C. The resulting mixture was stirred for additional 0.5 h at 0° C. To the above mixture was added methyl iodide (MeI) (948 mg, 6.68 mmol, 2.00 equiv) at 0° C. The resulting mixture was stirred for additional 2 h at room temperature then quenched by the addition of water (40 mL) at 0° C. The resulting mixture was extracted with ethyl acetate (EtOAc) (3×50 mL). The combined organic layers were washed with water (3×20 mL), and dried over anhydrous Na₂SO₄. After filtration, the filtrate was concentrated under reduced pressure and the residue was purified by silica gel chromatography (ethyl acetate/petroleum ether=3/1) (PE/EtOAc) to afford [(2R,3S)-3-methoxybutan-2-yl]bis[(4-methoxyphenyl)methyl]amine (600 mg, 76% yield). LCMS (ES, m/z)=344 [M+1]+.

Step 3: To a solution of [(2R,3S)-3-methoxybutan-2-yl]bis[(4-methoxyphenyl)methyl]amine (300 mg, 0.87 mmol, 1.00 equiv) in methanol (MeOH) (10 mL) was added Pd/C (10/o, 30 mg) under H₂ (gas). The resulting mixture was stirred for 2 h at room temperature under H₂ then filtered through a Celite pad and concentrated under reduced pressure. The crude (2R,3S)-3-methoxybutan-2-amine product was used in the next step directly without further purification. LCMS (ES, m/z)=104 [M+1]+.

Step 4: 3-Methoxy-4-(((2R,3S)-3-methoxybutan-2-yl)amino)-N-(5-(5-methyl-1H-pyrazol-1-yl)-1,3,4-thiadiazol-2-yl)-2-oxo-2H-pyran-6-carboxamide (Compound 166) was prepared according to Example 1, Part C, Step 2 using (2R,3S)-3-methoxybutan-2-amine as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent”. LCMS (ES, m/z)=435.15 [M+1]+. ¹H NMR (400 MHz, DMSO-d) δ 7.62 (d, J=1.6 Hz, 1H), 7.04 (s, 1H), 6.30 (d, J=1.6 Hz, 1H), 6.16 (br, 1H), 3.78-3.75 (m, 1H), 3.66 (s, 3H), 3.38-3.37 (m, 1H), 3.35 (s, 3H), 2.60 (s, 3H), 1.17 (d, J=6.8 Hz, 3H), 1.10 (d, J=6.4 Hz, 3H).

### Example 46: 4-((2-hydroxy-2-methylcyclopentyl)amino)-3-methoxy-N-(5-(5-methyl-1H-pyrazol-1-yl)-1,3,4-thiadiazol-2-yl)-2-oxo-2H-pyran-6-carboxamide (Compound 167, Rac-167), 4-(((1S,2R)-2-hydroxy-2-methylcyclopentyl)amino)-3-methoxy-N-(5-(5-methyl-1H-pyrazol-1-yl)-1,3,4-thiadiazol-2-yl)-2-oxo-2H-pyran-6-carboxamide (Compound 167a*) and 4-(((1R,2S)-2-hydroxy-2-methylcyclopentyl)amino)-3-methoxy-N-(5-(5-methyl-1H-pyrazol-1-yl)-1,3,4-thiadiazol-2-yl)-2-oxo-2H-pyran-6-carboxamide (Compound 167b*)

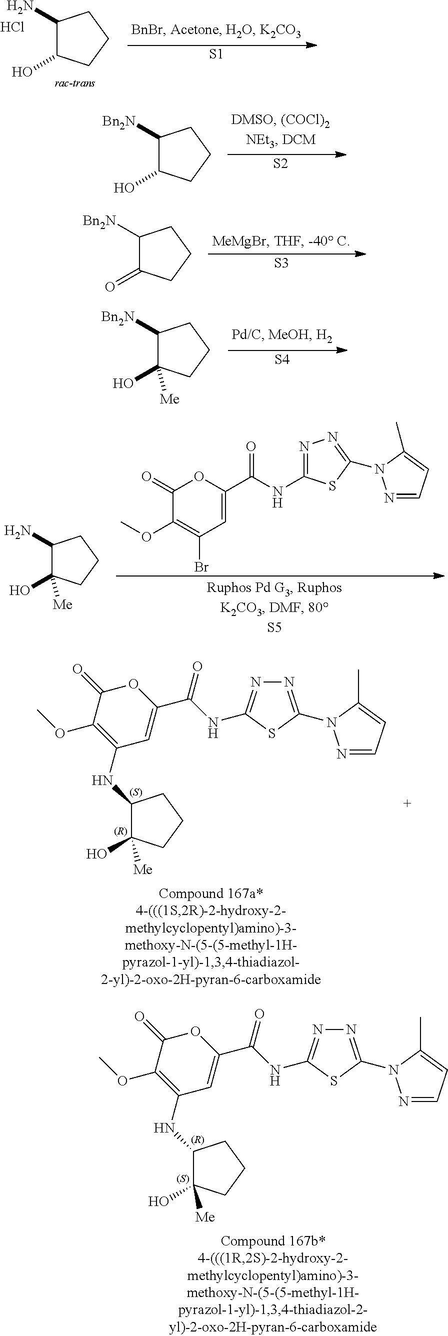

Step 1: To a stirred solution of racemic-trans-2-aminocyclopentan-1-ol (4.00 g, 39.54 mmol, 1.00 equiv) and benzyl bromide (BnBr) (16.3 g, 95.3 mmol, 2.41 equiv) in acetone (30 mL) and H₂O (30 mL) was added K₂CO₃ (1.09 g, 79.1 mmol, 2.00 equiv) at room temperature. The resulting mixture was stirred for 5 h at 60° C. under N₂ (nitrogen gas) then concentrated under vacuum. The mixture was extracted with ethyl acetate (EtOAc) (3×50 mL). The combined organic layers were washed with brine (3×10 mL), and dried over anhydrous Na₂SO₄. After filtration, the filtrate was concentrated under reduced pressure and the material was purified by C18 reverse phase flash chromatography (acetonitrile (MeCN):H₂O=1:1) to afford trans-2-(dibenzylamino)cyclopentan-1-ol (5.00 g, 40% yield) as a yellow oil. LCMS (ESI, m/z)=282 [M+1]+.

Step 2: Oxalic dichloride (2.70 g, 21.3 mmol, 1.20 equiv) was dissolved in dichloromethane (DCM) (50 mL) at room temperature and stirred for 5 min at −60° C. under N₂ (nitrogen gas). Then dimethylsulfoxide (DMSO) (1.66 g, 21.3 mmol, 1.20 equiv) was added dropwise at −60° C. under N₂. The resulting mixture was stirred for 30 min at −60° C. under N₂ then trans-2-(dibenzylamino)cyclopentan-1-ol (5.00 g, 17.8 mmol, 1.00 equiv) in tetrahydrofuran (THF) (5 ml) added dropwise over 5 min at −60° C. The resulting mixture was stirred for additional 1 h at room temperature then quenched by the addition of water (50 mL) at 0° C. The mixture was then extracted with DCM (100 mL). The organic layer was washed with brine (90 mL), and dried over anhydrous Na₂SO₄. After filtration, the filtrate was concentrated under reduced pressure and the crude material was purified by C18 reverse phase flash chromatography (MeCN:H₂O=1:1) to afford 2-(dibenzylamino)cyclopentan-1-one (2.40 g, 45% yield). LCMS (ESI, m/z)=280.0 [M+1]+.

Step 3: To a stirred solution of 2-(dibenzylamino)cyclopentan-1-one (2.40 g, 8.59 mmol, 1.00 equiv) in THF (50 mL) was added methyl magnesium bromide (MeMgBr) (43.0 mL of 1 M in THF, 5.00 equiv) dropwise at −40° C. under N₂ (nitrogen gas). The resulting mixture was stirred for 2 h at 0° C. under N₂. The reaction was quenched with water (100 mL) at room temperature. The resulting mixture was filtered and the filter cake was washed with diethyl ether (Et₂O) (3×100 mL). The filtrate was concentrated under reduced pressure. The residue obtained was extracted with ethyl acetate (EtOAc) (3×50 mL), the combined organic layers were washed with brine (50 mL), and dried over anhydrous Na₂SO₄. After filtration, the filtrate was concentrated under reduced pressure and the residue was purified by C18 reverse phase flash chromatography (MeCN:H₂O=4:1) to afford cis-2-(dibenzylamino)-1-methylcyclopentan-1-ol (900 mg, 32% yield). LCMS (ESI, m/z)=296.0 [M+1]+.

Step 4: To a stirred solution of cis-2-(dibenzylamino)-1-methylcyclopentan-1-ol (800 mg, 2.70 mmol, 1.00 equiv) in 5.00 mL methanol (MeOH) was added Pd/C (10%, 800 mg). The mixture was stirred at room temperature for 5 h under H₂ (gas). The mixture was filtered and the filtrate was concentrated under vacuum to provide crude cis-(1-methyl, 2-amino)-cyclopentan-1-ol (200 mg) which was used in the next step directly without further purification. LCMS (EST, mli)=116.0 [M+1]+.

Step 5: 4-(((1S,2R)-2-Hydroxy-2-methylcyclopentyl)amino)-3-methoxy-N-(5-(5-methyl-1H-pyrazol-1-yl)-1,3,4-thiadiazol-2-yl)-2-oxo-2H-pyran-6-carboxamide (Compound 167a*) and 4-(((1R,2S)-2-hydroxy-2-methylcyclopentyl)amino)-3-methoxy-N-(5-(5-methyl-H-pyrazol-1-yl)-1,3,4-thiadiazol-2-yl)-2-oxo-2H-pyran-6-carboxamide (Compound 167b*) were prepared as a racemic mixture according to Example 1, Part C, Step 2 using cis-(1-methyl,2-amino)-cyclopentan-1-ol as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent”. LCMS (ES, m/z)=447.0 [M+1]+; ¹H NMR (400 MHz, DMSO-d) δ 13.32 (br, 1H), 7.79 (d, J=1.6 Hz, 1H), 7.40 (s, 1H), 6.44 (d, J=1.6 Hz, 1H), 6.04 (br, 1H), 4.98 (br, 1H), 3.74 (s, 3H), 2.68 (s, 3H), 2.13-2.03 (m, 1H), 1.80-1.70 (m, 3H), 1.67-1.69 (m, 2H), 1.21 (s, 3H).

### Example 47: 4-((2-cyclopropoxyethyl)amino)-3-methoxy-N-(5-(5-methyl-1H-pyrazol-1-yl)-1,3,4-thiadiazol-2-yl)-2-oxo-2H-pyran-6-carboxamide (Compound 168)

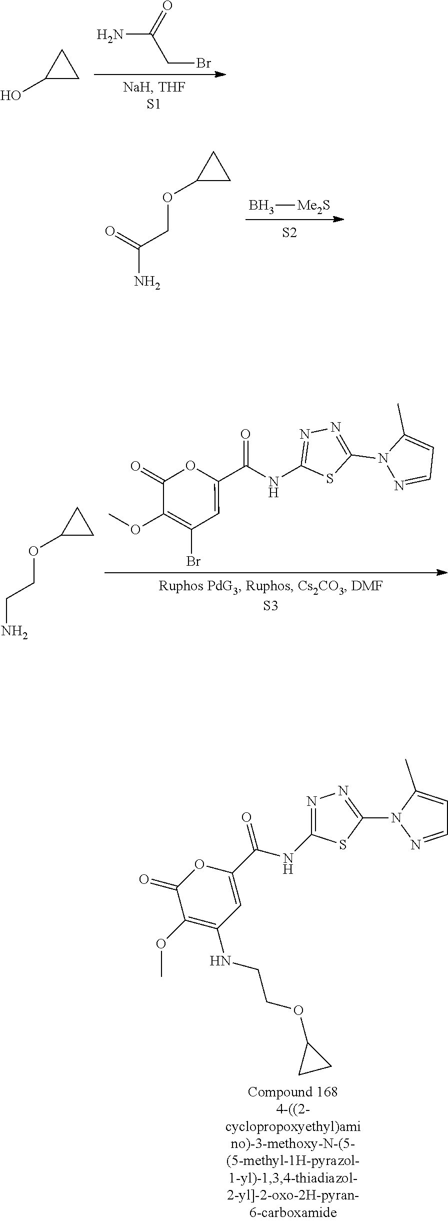

Step 1: To a stirred solution of cyclopropanol (1.00 g, 17.2 mmol, 1.00 equiv) in tetrahydrofuran (THF) was added sodium hydride (60% in mineral oil, 0.62 g) at 0° C. The mixture was stirred for 30 min then bromoacetamide (2.14 g, 15.50 mmol, 0.90 equiv) was added and the mixture was allowed to warm to rt and stirred for 1 h. The mixture was quenched by water and extracted with dichloromethane (DCM) (3×25 mL). The combined organic layers were dried over anhydrous Na₂SO₄, filtered and the filtrate was concentrated under vacuum. The resulting residue was purified by C18 reverse phase flash chromatography (H₂O:acetonitrile (MeCN)=10:1) to provide 2-cyclopropoxyacetamide (700 mg, 35% yield).

Step 2: 2-cyclopropoxyacetamide (700 mg, 6.08 mmol, 1.00 equiv) was dissolved in BH₃-Me₂S (7.00 mL of 10 M solution, 7.00 mmol, 1.15 equiv.) at room temperature. The resulting mixture was stirred for 12 h at 60° C. under air atmosphere then concentrated under reduced pressure. The residue was purified by prep-Thin Layer Chromatography (prep-TLC) (dichloromethane (DCM):MeOH=10:1) to afford 2-cyclopropoxyethanamine (30 mg, 5% yield).

Step 3: 4-(2-Cyclopropoxyethylamino)-5-methoxy-N-(5-(5-methyl-1H-pyrazol-1-yl)-1,3,4-thiadiazol-2-yl)-6-oxo-6H-pyran-2-carboxamide (Compound 168) was prepared according to Example 1, Part C, Step 2 using 2-cyclopropoxyethanamine as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent”. LCMS (ES, m/z)=433.0 [M+1]+; ¹H NMR (400 MHz, DMSO-d₆) δ 7.62 (d, J=1.6 Hz, 1H), 7.02 (s, 1H), 6.70 (t, J=6.0 Hz, 1H), 6.31 (d, J=1.6 Hz, 1H), 3.64 (s, 3H), 3.62-3.57 (m, 2H), 3.45-3.42 (m, 2H), 2.60 (s, 3H), 0.60-0.33 (m, 4H).

### Example 48: 4-(((1R, 2S)-2-(2,2-difluoroethoxy)cyclopentyl)amino)-3-methoxy-N-(5-(5-methyl-1H-pyrazol-1-yl)-1,3,4-thiadiazol-2-yl)-2-oxo-2H-pyran-6-carboxamide (Compound 171)

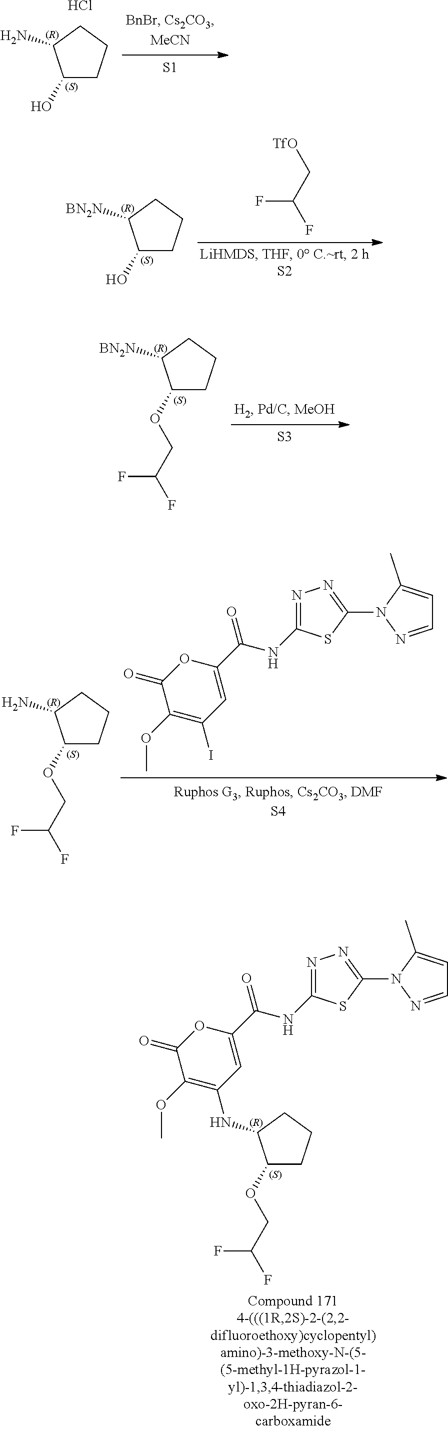

Step 1: Into a solution of (1S,2R)-2-aminocyclopentan-1-ol hydrochloride (1.83 g, 13.35 mmol, 1.0) equiv) in acetonitrile (MeCN) (50 mL) was added benzyl bromide (BnBr) (6.30 g, 36.8 mmol, 2.76 equiv) and cesium carbonate (12.7 g, 39.0 mmol, 2.92 equiv) at room temperature. The resulting mixture was stirred for 2 h at 60° C. The precipitated solids were collected by filtration and washed with acetonitrile (3×10 mL) and the filtrate was concentrated under reduced pressure. The resulting residue was purified by silica gel column chromatography, eluted with petroleum ether/ethyl acetate (PE/EtOAc) (1:1), to afford (1S,2R)-2-(dibenzylamino)cyclopentan-1-ol (2.1 g, 48% yield). LCMS (ES, m/z)=282.2 [M+1]+.

Step 2: Into a solution of (1S,2R)-2-(dibenzylamino)cyclopentan-1-ol (400 mg, 1.42 mmol, 1.00 equiv) in tetrahydrofuran (THF) (5 mL) was added lithium bis(trimethylsilyl)amide (LiHMDS) (2.80 mL, 2.80 mmol, 1.97 equiv) dropwise at 0° C. The mixture was stirred for 15 min at room temperature then to this was added 2,2-difluoroethyl trifluoromethanesulfonate (600 mg, 2.80 mmol, 1.97 equiv) dropwise at 0° C. The resulting mixture was stirred for additional 2 h at room temperature then quenched with sat. NH₄Cl (aq.) at room temperature. The aqueous layer was extracted with ethyl acetate (EtOAc) (3×20 mL) and concentrated under reduced pressure. The resulting residue was purified by silica gel column chromatography, eluted with petroleum ether/ethyl acetate (PE/EtOAc) (1:1), to afford (1R,2S)—N,N-dibenzyl-2-(2,2-difluoroethoxy)cyclopentan-1-amine (260 mg, 47% yield). LCMS (ES, m/z)=346.2 [M+1]+. Tf=triflate.

Step 3: Into a solution of (1R,2S)—N,N-dibenzyl-2-(2,2-difluoroethoxy)cyclopentan-1-amine (200 mg, 0.579 mmol, 1.00 equiv) in methanol (MeOH) (4 mL) was added Pd/C (800 mg, 7.52 mmol, 13.0 equiv) at room temperature. The resulting mixture was stirred for 1 h at room temperature under H₂ (gas). The mixture was then filtered and the filter cake was washed with methanol (MeOH) (3×4 mL). The filtrate was concentrated under reduced pressure to provide (1R,2S)-2-(2,2-difluoroethoxy) cyclopentane-1-amine (40 mg, 42% yield) which was used directly without further purification.

Step 4: 4-(((1R, 2S)-2-(2,2-Difluoroethoxy)cyclopentyl)amino)-3-methoxy-N-(5-(5-methyl-1H-pyrazol-1-yl)-1,3,4-thiadiazol-2-yl)-2-oxo-2H-pyran-6-carboxamide (Compound 171) was prepared according to Example 6, Step 5 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and (1R,2S)-2-(2,2-difluoroethoxy)cyclopentan-1-amine as the “amine reagent”. LCMS (ES, m/z)=497.1 [M+1]+. ¹H NMR (400 MHz, DMSO-d6) δ 7.70 (s, 1H), 7.24 (s, 1H), 6.37 (s, 1H), 6.14-5.93 (m, 2H), 4.10-4.07 (m, 1H), 4.03-3.96 (m, 1H), 3.86-3.66 (m, 2H), 3.65 (s, 3H), 2.64 (s, 3H), 2.05-1.95 (m, 1H), 1.93-1.70 (m, 3H), 1.69-1.50 (m, 2H).

### Example 49: 4-(((1S,2R)-2-(2,2-difluoroethoxy)cyclobutyl)amino)-3-methoxy-N-(5-(5-methyl-1H-pyrazol-1-yl)-1,3,4-thiadiazol-2-yl)-2-oxo-2H-pyran-6-carboxamide (Compound 172*) and 4-(((1R,2S)-2-(2,2-difluoroethoxy)cyclobutyl)amino)-3-methoxy-N-(5-(5-methyl-1H-pyrazol-1-yl)-1,3,4-thiadiazol-2-yl)-2-oxo-2H-pyran-6-carboxamide (Compound 173*)

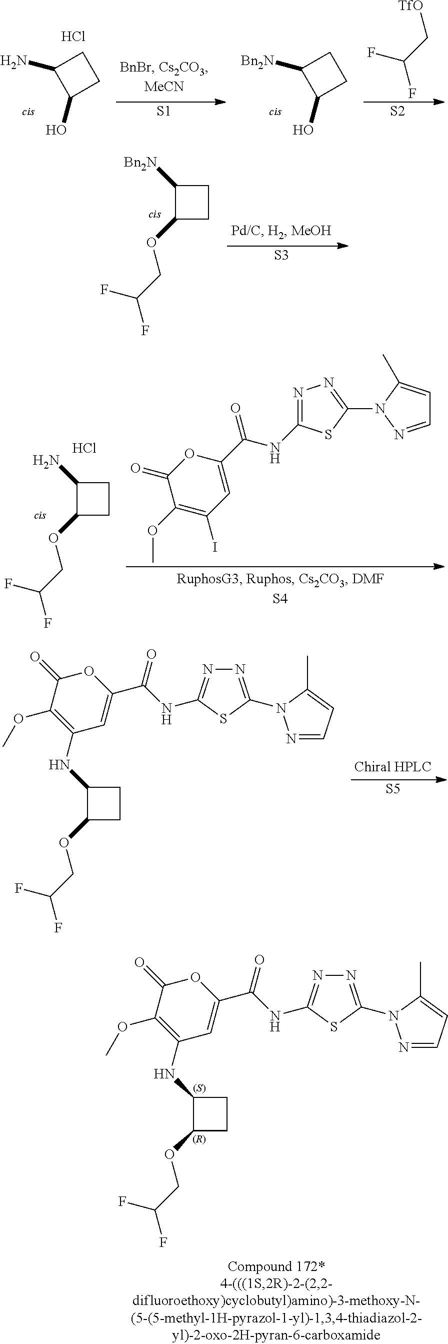

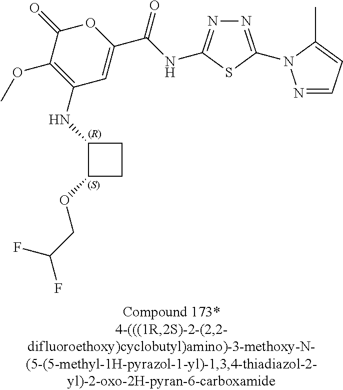

Racemic 4-(((1,2-cis)-2-(2,2-difluoroethoxy)cyclobutyl)amino)-3-methoxy-N-(5-(5-methyl-1H-pyrazol-1-yl)-1,3,4-thiadiazol-2-yl)-2-oxo-2H-pyran-6-carboxamide was prepared from cis-2-aminocyclobutan-1-ol hydrochloride according to the procedure outlined in Example 48, Steps 1-4, using rac-(1,2-cis)-2-aminocyclobutan-1-ol hydrochloride instead of (1S,2R)-2-aminocyclopentan-1-ol hydrochloride to provide (1,2-cis)-2-(2,2-difluoroethoxy)cyclobutan-1-amine (“amine reagent”), followed by coupling with 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6; “halo-pyrone reagent”). Separation of constituent enantiomers by Chiral-HPLC (Chiral ART Cellulose-SA, 2*25 cm, 5 μm; Mobile Phase A: Hexanes (0.1% trifluoroacetic acid (TFA)). Mobile Phase B: ethanol (EtOH):dichloromethane (DCM)=1:1; Flow rate: 20 mL/min; Gradient: 70% B to 70% B in 8.5 min; Wave Length: 220/254 nm; RT1 (min): 4.86; RT2 (min): 7.61; Sample Solvent: methanol (MeOH):DCM=1:1) provided two enantiomers with arbitrarily assigned stereochemistry: 4-(((1S,2R)-2-(2,2-difluoroethoxy)cyclobutyl)amino)-3-methoxy-N-(5-(5-methyl-1H-pyrazol-1-yl)-1,3,4-thiadiazol-2-yl)-2-oxo-2H-pyran-6-carboxamide 2,2,2-trifluoroacetate (Compound 172*), first eluting peak. LCMS (ES, m/z)=483.10 [M+1]+. ¹H NMR (400 MHz, DMSO-d6) δ 13.37 (br, 1H), 7.79 (d, J=1.6 Hz, 1H), 7.29 (s, 1H), 6.72 (br, 1H), 6.45 (d, J=1.6 Hz, 1H), 6.11 (t, J=84.0 Hz, 1H), 4.39-4.30 (m, 2H), 3.76 (s, 3H), 3.75-3.55 (m, 2H), 2.68 (s, 3H), 2.14-2.02 (m, 4H); and 4-(((1R,2S)-2-(2,2-difluoroethoxy)cyclobutyl)amino)-3-methoxy-N-(5-(5-methyl-1H-pyrazol-1-yl)-1,3,4-thiadiazol-2-yl)-2-oxo-2H-pyran-6-carboxamide 2,2,2-trifluoroacetate (Compound 173*), second eluting peak, LCMS (ES, m/z)=483.05 [M+1]+, ¹H NMR (400 MHz, DMSO-d6) δ 13.37 (br, 1H), 7.79 (d, J=1.6 Hz, 1H), 7.29 (s, 1H), 6.72 (br, 1H), 6.45 (d, J=1.6 Hz, 1H), 6.11 (t, J=84.0 Hz, 1H), 4.39-4.30 (m, 2H), 3.76 (s, 3H), 3.75-3.55 (m, 2H), 2.68 (s, 3H), 2.14-2.02 (m, 4H).

### Example 50: (R)-4-(2-(cyanomethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 174) and (R)-4-(2-(hydroxymethyl)pyrrolidin-1-yl)-3-methoxy-N-(5-(S-methyl-1H-pyrazol-1-yl)-1,3,4-thiadiazol-2-yl)-2-oxo-2H-pyran-6-carboxamide (Compound 174-OH)

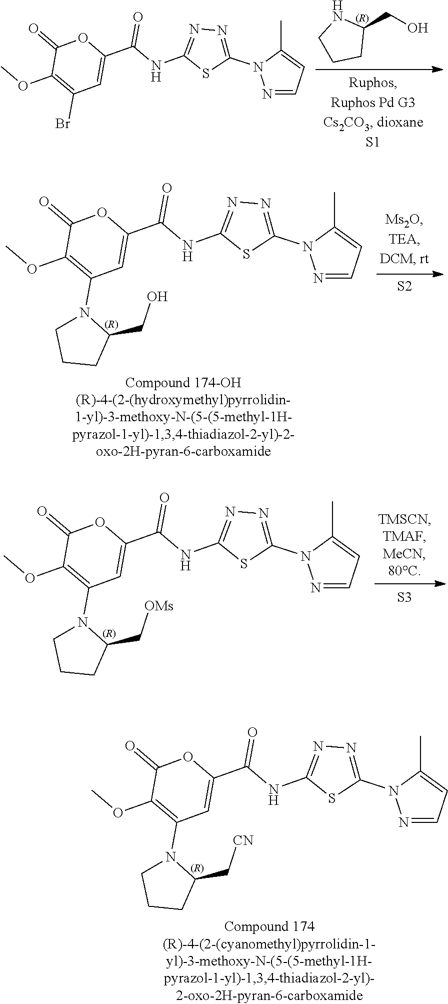

Step 1: Into a 30 mL sealed tube were added 4-bromo-3-methoxy-N-(5-(5-methyl-1H-pyrazol-1-yl)-1,3,4-thiadiazol-2-yl)-2-oxo-2H-pyran-6-carboxamide (product of Example 1, Part C, Step 1) (100 mg, 0.25 mmol, 1 equiv), (R)-prolinol (30.5 mg, 0.30 mmol, 1.20 equiv), N,N-dimethylformamide (DMF) (5.0 mL), CS₂CO₃ (164 mg, 0.500 mmol, 2.00 equiv), 2-Dicyclohexylphosphino-2′,6′-diisopropoxybiphenyl (RuPhos) (35.16 mg, 0.07 mmol, 0.30 equiv) and (2-dicyclohexylphosphino-2′,6′-diisopropoxy-1,1′-biphenyl)[2-(2′-amino-1,1′-biphenyl)]palladium(I) methanesulfonate (RuPhos Palladacycle Gen3 (42 mg, 0.050 mmol, 0.20 equiv) at room temperature. The resulting mixture was stirred for 1 h at 80° C. under nitrogen atmosphere. The reaction was monitored by LCMS. The reaction was quenched with water (10 mL) at room temperature. The resulting mixture was extracted with ethyl acetate (EtOAc) (3×10 mL). The organic layers were washed with water (3×10 mL), dried over anhydrous Na₂SO₄ and was concentrated under reduced pressure. The crude residue was purified by reversed-phase flash chromatography with the following conditions: column, C18; mobile phase, ACN in water, 23% to 23% gradient in 10 min; detector, UV 254 nm to afford (R)-4-(2-(hydroxymethyl)pyrrolidin-1-yl)-3-methoxy-N-(5-(5-methyl-JH-pyrazol-1-yl)-1,3,4-thiadiazol-2-yl)-2-oxo-2H-pyran-6-carboxamide (30 mg, purity=85%). The product was further purified by Prep-HPLC with the following conditions (Column: XBridge Shield RP18 OBD Column, 30*150 mm, 5 μm; Mobile Phase A: water (10 mmol/L NH₄HCO₃). Mobile Phase B: ACN; Flow rate: 60 mL/min; Gradient: 15% B to 25% B in 10 min, 25% B; Wave Length: 254 nm; RT1 (min): 8.5) to afford (R)-4-(2-(hydroxymethyl)pyrrolidin-1-yl)-3-methoxy-N-(5-(5-methyl-JH-pyrazol-1-yl)-1,3,4-thiadiazol-2-yl)-2-oxo-2H-pyran-6-carboxamide (Compound 174-OH) (15.4 mg, 14% yield). LCMS (ES, m/z)=433.0 [M+1]+. ¹H NMR (400 MHz, Methanol-d₄) δ 7.64 (s, 1H), 7.30 (s, 1H), 6.35 (s, 1H), 4.35 (s, 1H), 3.94 (s, 1H), 3.72-3.62 (t, J=4.0 Hz, 3H), 3.60-3.51 (m, 3H), 3.35 (s, 1H), 2.75-2.62 (t, J=18.2 Hz, 3H), 2.05-1.95 (m, 4H). The H of the —OH in the ¹HNMR was not detected.

Step 2: To a stirred solution of (R)-4-(2-(hydroxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 174-OH) (240 mg, 0.555 mmol, 1.00 equiv) in dichloromethane (DCM) (10 mL) was added triethylamine (TEA) (36 mg, 0.356 mmol, 0.64 equiv) and methanesulfonic anhydride (Ms₂O) (192 mg, 1.102 mmol, 1.99 equiv) at room temperature. The resulting mixture was stirred overnight at room temperature then concentrated under vacuum. The residue was then purified by silica gel column chromatography, eluted with petroleum ether/ethyl acetate (PE/EtOAc) (1:1), to afford (R)-(1-(3-methoxy-6-((5-(5-methyl-1H-pyrazol-1-yl)-1,3,4-thiadiazol-2-yl)carbamoyl)-2-oxo-2H-pyran-4-yl)pyrrolidin-2-yl)methyl methanesulfonate (100 mg, 35% yield). LCMS (ES, m/z)=511 [M+1]+.

Step 3: To a stirred solution of (R)-(1-(3-methoxy-6-((5-(5-methyl-1H-pyrazol-1-yl)-1,3,4-thiadiazol-2-yl)carbamoyl)-2-oxo-2H-pyran-4-yl)pyrrolidin-2-yl)methyl methanesulfonate (25 mg, 0.049 mmol, 1.00 equiv) in acetonitrile (MeCN) (3 mL) was added trimethylsilyl cyanide (TMSCN) (58 mg, 0.585 mmol, 11.9 equiv) and tetramethylammonium fluoride (TMAF) (18 mg, 0.193 mmol, 3.95 equiv) dropwise at room temperature. The resulting mixture was stirred overnight at 80° C. then concentrated under reduced pressure. The crude product was purified by Prep-HPLC (XBridge Prep Phenyl OBD Column, 19*250 mm, 5 μm; Mobile Phase A: water (10 mmol/L NH₄HCO₃), Mobile Phase B: acetonitrile (MeCN); Flow rate: 25 mL/min; Gradient: 20% B to 30% B in 10 min. 30% B; Wave Length: 254 nm; RT1 (min): 9) to afford (R)-4-(2-(cyanomethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 174) (7.9 mg, 36% yield). LCMS (ES, m/z)=442.10 [M+1]+. 1H NMR (400 MHz, DMSO-d6) δ 7.72 (d, J=1.6 Hz, 1H), 7.17 (s, 1H), 6.40 (d, J=1.6 Hz, 1H), 4.54-4.46 (m, 1H), 3.81-3.76 (m, 1H), 3.69 (s, 3H), 3.58-3.51 (m, 1H), 2.83-2.78 (m, 2H), 2.64 (s, 3H), 2.21-2.05 (m, 2H), 1.98-1.87 (m, 2H).

### Example 51: N-(5-(5-amino-1H-pyrazol-1-yl)-1,3,4-thiadiazol-2-yl)-4-(bicyclo[1.1.1]pentan-1-ylamino)-3-(2-hydroxyethoxy)-2-oxo-2H-pyran-6-carboxamide (Compound 177) and N-(5-(5-acetamido-1H-pyrazol-1-yl)-1,3,4-thiadiazol-2-yl)-4-(bicyclo[1.1.1]pentan-1-ylamino)-3-(2-(tert-butoxy)ethoxy)-2-oxo-2H-pyran-6-carboxamide (Compound 177-Ac-OtBu)

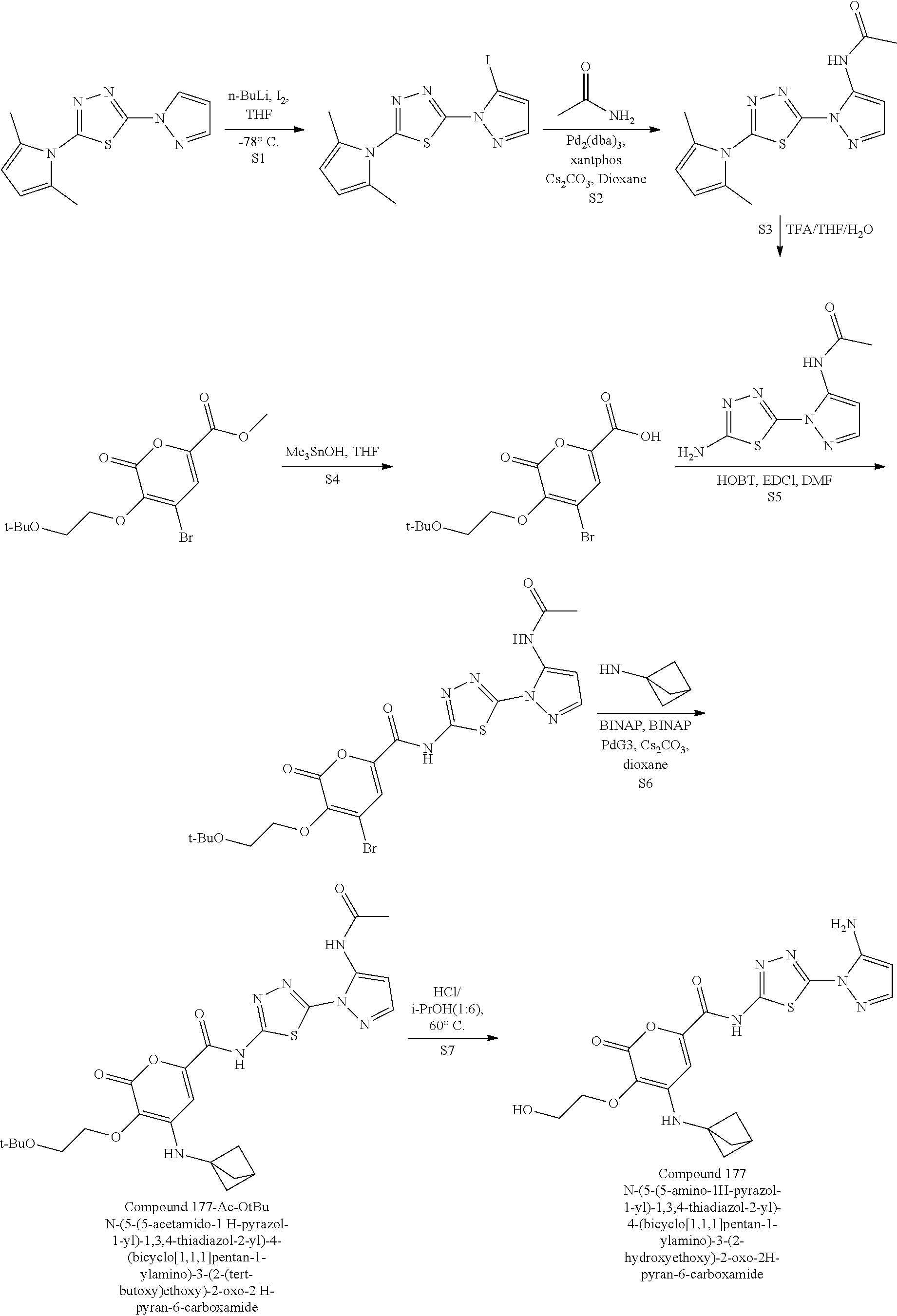

Step 1: To a stirred solution of 2-(2,5-dimethylpyrrol-1-yl)-5-(pyrazol-1-yl)-1,3,4-thiadiazole (product of Example 1, Part B, Step 2) (6.00 g, 24.46 mmol, 1.00 equiv) in tetrahydrofuran (THF) (70 mL) was added n-butyl lithium (n-BuLi) (11.74 mL, 29.35 mmol, 1.20 equiv, 2.5 M) in portions at −78° C. under N₂ (nitrogen gas). The resulting mixture was stirred for 1 h at −78° C. under N₂. And then to the above mixture was added I₂ (18.62 g, 73.38 mmol, 3.00 equiv) in THF (20 mL) in portions over 30 min at −78° C. under N₂. The resulting mixture was stirred for additional 40 min at −78° C., and then the resulting mixture was stirred for 2 h at room temperature under N₂. The reaction was then quenched with sat. NH₄Cl (aq.) at room temperature. The mixture was extracted with ethyl acetate (EtOAc) (3×100 mL), washed with sat. Na₂S₂O₃ (2×100 mL), then with brine (2×100 mL), and dried over anhydrous Na₂SO₄. After filtration, the filtrate was concentrated under reduced pressure. The residue was purified by silica gel column chromatography, eluted with petroleum ether/ethyl acetate (PE/EtOAc) (15:1), to afford 2-(2,5-dimethylpyrrol-1-yl)-5-(5-iodopyrazol-1-yl)-1,3,4-thiadiazole (6.8 g, 75% yield). LCMS (ES, m/z)=372 [M+1]+.

Step 2: A mixture of 2-(2,5-dimethylpyrrol-1-yl)-5-(5-iodopyrazol-1-yl)-1,3,4-thiadiazole (2.00 g, 5.39 mmol, 1.00 equiv), tris(dibenzylidenaceton)dipalladium(0) dibenzylidenacetone (Pd₂(dba)₃) (494 mg, 0.539 mmol, 0.10 equiv), (9,9-Dimethyl-9H-xanthene-4,5-diyl)bis(diphenylphosphane) (XantPhos) (624 mg, 1.078 mmol, 0.20 equiv), Cs₂CO₃ (3.53 g, 10.8 mmol, 2.01 equiv) and acetamide (962 mg, 16.3 mmol, 3.02 equiv) in dioxane (25 mL) was stirred for 1 h at 120° C. under N₂ then concentrated under vacuum. The resulting residue was purified by silica gel column chromatography, eluted with petroleum ether/ethyl acetate (PE/EtOAc) (8:1), to afford N-(2-[5-(2,5-dimethylpyrrol-1-yl)-1,3,4-thiadiazol-2-yl]pyrazol-3-yl)acetamide (1.1 g, 67% yield). LCMS (ES, m/z)=303 [M+1]+.

Step 3: To a stirred solution of N-{2-[5-(2,5-dimethylpyrrol-1-yl)-1,3,4-thiadiazol-2-yl]pyrazol-3-yl}acetamide (1.10 g, 3.64 mmol, 1.00 equiv) in tetrahydrofuran (THF) (4 mL) and H₂O (4 mL) was slowly added trifluoroacetic acid (TFA) (8 mL) dropwise at 0° C. The resulting mixture was stirred overnight at room temperature then volatiles removed under a stream of N₂. The residue was then diluted with dichloromethane (DCM) (3 mL). The precipitated solids were collected by filtration and washed with DCM (2×1 mL) to afford N-[2-(5-amino-1,3,4-thiadiazol-2-yl)pyrazol-3-yl]acetamide (400 mg, 49% yield). LCMS (ES, m/z)=225 [M+1]+.

Step 4: To a stirred solution of methyl 4-bromo-5-[2-(tert-butoxy)ethoxy]-6-oxopyran-2-carboxylate (prepared following Example 15, Step 1, but using 2-tertbutoxyethanol instead of 2-methoxyethanol) (3.0 g, 8.59 mmol, 1.00 equiv) in tetrahydrofuran (THF) (30 mL) was added trimethyltin hydroxide (4.00 g, 22.12 mmol, 2.57 equiv) at room temperature. The resulting mixture was stirred for 1 h at room temperature then concentrated under reduced pressure. The resulting residue was purified by C18 reverse phase flash chromatography (acetonitrile (MeCN) in water, 0% to 100% gradient in 10 min) to provide 4-bromo-5-[2-(tert-butoxy)ethoxy]-6-oxopyran-2-carboxylic acid (2.4 g, 83% yield). LCMS (ES, m/z)=335 [M+1]+.

Step 5: To a stirred solution of 4-bromo-5-[2-(tert-butoxy)ethoxy]-6-oxopyran-2-carboxylic acid (550 mg, 1.64 mmol, 1.00 equiv) in N,N-dimethylformamide (DMF) (6 mL) was added hydroxybenzotriazole (HOBT) (445 mg, 3.29 mmol, 2.01 equiv), 1-ethyl-3-(3-dimethylaminopropyl)carbodiimide (EDCI) (943 mg, 4.92 mmol, 3.00 equiv) and N-[2-(5-amino-1,3,4-thiadiazol-2-yl)pyrazol-3-yl]acetamide (product of Step 3 of this Example) (440 mg, 1.96 mmol, 1.20 equiv) at room temperature. The resulting mixture was stirred for 1.5 h at room temperature. The precipitated solids were then collected by filtration and washed with acetonitrile (MeCN) (3×5 mL) to provide 4-bromo-5-[2-(tert-butoxy)ethoxy]-N-[5-(5-acetamidopyrazol-1-yl)-1,3,4-thiadiazol-2-yl]-6-oxopyran-2-carboxamide (377 mg, 42% yield). LCMS (ES, m/z)=541 [M+1]+.

Step 6: To a stirred solution of 4-bromo-5-[2-(tert-butoxy)ethoxy]-N-[5-(5-acetamidopyrazol-1-yl)-1,3,4-thiadiazol-2-yl]-6-oxopyran-2-carboxamide (“halo-pyrone reagent”) (250 mg, 0.462 mmol, 1.00 equiv) in dioxane (3 mL) was added ([1,1′-binaphthalene]-2,2′-diyl)bis(diphenylphosphane) (BINAP) (61 mg, 0.098 mmol, 0.21 equiv), [2′-(diphenylphosphanyl)-[1,1′-binaphthalen]-2-yl]diphenylphosphane; [2′-amino-[1,1′-biphenyl]-2-yl]palladio methanesulfonate (BINAP Pd G3) (101 mg, 0.102 mmol, 0.22 equiv), cesium carbonate (400 mg, 1.23 mmol, 2.66 equiv) and bicyclo[1.1.1]pentan-1-amine (“amine reagent”) (100 mg, 1.20 mmol, 2.60 equiv) at room temperature. The resulting mixture was stirred for 3 h at 100° C. under N₂ then concentrated under vacuum. The resulting residue was purified by C18 reverse phase flash chromatography (MeCN in water, 0% to 100% gradient in 10 min; detector, UV 254 nm (MeCN:H₂O=2:8) to provide 4-[bicyclo[1.1.1]pentan-1-ylamino]-5-[2-(tert-butoxy) ethoxy]-N-[5-(5-acetamidopyrazol-1-yl)-1,3,4-thiadiazol-2-yl]-6-oxopyran-2-carboxamide (also referred to herein as N-(5-(5-acetamido-1H-pyrazol-1-yl)-1,3,4-thiadiazol-2-yl)-4-(bicyclo[1.1.1]pentan-1-ylamino)-3-(2-tert-butoxy)ethoxy)-2-oxo-2H-pyran-6-carboxamide, Compound 177-Ac-OtBu) (100 mg, 40% yield). LCMS (ES, m/z)=544 [M+1]+.

Step 7: A solution of 4-[bicyclo[1.1.1]pentan-1-ylamino]-5-[2-(tert-butoxy)ethoxy]-N-[5-(5-acetamidopyrazol-1-yl)-1,3,4-thiadiazol-2-yl]-6-oxopyran-2-carboxamide (80 mg, 0.15 mmol, 1.00 equiv) in conc. HCl (0.4 mL) and isopropyl alcohol (r-PrOH) (2.4 mL) was stirred for 14 h at 60° C. then concentrated under reduced pressure. The residue was then purified by C18 reverse phase flash chromatography (MeCN in water (0.1% formic acid (FA)), 0% to 100% gradient in 10 min (MeCN:H₂O=2:8) then further purified by Prep-HPLC (XBridge Prep OBD C18 Column, 30*150 mm, 5 μm; Mobile Phase A: Water (10 mmol/L NH₄HCO₃), Mobile Phase B: acetonitrile (MeCN); Flow rate: 60 mL/min; Gradient: 16% B to 24% B in 8 min, 24% B; Wave Length: 220/254 nm; RT1 (min): 6.17) to afford N-(5-(5-amino-1H-pyrazol-1-yl)-1,3,4-thiadiazol-2-yl)-4-(bicyclo[1.1.1]pentan-1-ylamino)-3-(2-hydroxyethoxy)-2-oxo-2H-pyran-6-carboxamide (Compound 177) (9.8 mg, 15% yield). LCMS (ES, m/z)=446.05 [M+1]+. ¹H NMR (400 MHz, DMSO-d6) δ 7.40 (d, J=2.0 Hz, 2H), 7.26 (s, 1H), 6.63 (s, 2H), 5.42 (d, J=2.0 Hz, 1H), 5.22-5.08 (m, 1H), 3.96-3.84 (m, 2H), 3.67-3.54 (m, 2H), 2.54 (s, 1H), 2.16 (s, 6H).

### Example 52: N-(5-(5-amino-1H-pyrazol-1-yl)-1,3,4-thiadiazol-2-yl)-4-(bicyclo[1.1.1]pentan-1-ylamino)-3-methoxy-2-oxo-2H-pyran-6-carboxamide (Compound 178) and N-(5-(5-acetamido-1H-pyrazol-1-yl)-1,344-thiadiazol-2-yl)-4-(bicyclo[1.1.1]pentan-1-ylamino)-3-methoxy-2-oxo-2H-pyran-6-carboxamide (Compound 178-Ac)

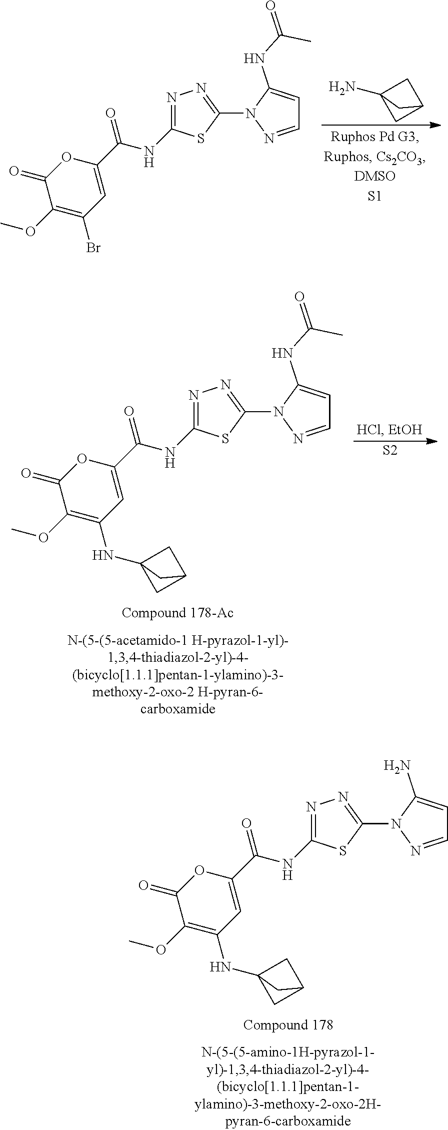

Step 1: N-(5-(5-Acetamido-1H-pyrazol-1-yl)-1,3,4-thiadiazol-2-yl)-4-(bicyclo[1.1.1]pentan-1-ylamino)-3-methoxy-2-oxo-2H-pyran-6-carboxamide (Compound 178-Ac) was prepared according to Example 1, Part C, Step 2 using N-(5-(5-acetamido-1H-pyrazol-1-yl)-1,3,4-thiadiazol-2-yl)-4-bromo-3-methoxy-2-oxo-2H-pyran-6-carboxamide (also referred to herein as 4-bromo-N-[5-(5-acetamidopyrazol-1-yl)-1,3,4-thiadiazol-2-yl]-5-methoxy-6-oxopyran-2-carboxamide; the “halo-pyrone reagent”; product of Example 56, Step 1) and bicyclo[1.1.1]pentan-1-amine as the “amine reagent”. LCMS (ES, m/z)=447 [M+1]+.

Step 2: A solution of N-(5-(5-acetamido-1H-pyrazol-1-yl)-1,3,4-thiadiazol-2-yl)-4-(bicyclo[1.1.1]pentan-1-ylamino)-3-methoxy-2-oxo-2H-pyran-6-carboxamide (Compound 178-Ac) (23 mg, 0.050 mmol, 1.00 equiv) in conc. HCl (0.2 mL) and ethanol (EtOH) (1.2 mL) was stirred for 4 h at 60° C. The mixture was then filtered, and the filter cake was washed with EtOH (3×2 mL). The filtrate was concentrated under reduced pressure and the resulting residue purified by Prep-HPLC (XBridge Prep Phenyl OBD Column, 19*150 mm, 5 μm; Mobile Phase A: water (10 mmol/L NH₄HCO₃), Mobile Phase B: acetonitrile (MeCN); Flow rate: 60 mL/min; Gradient: 14% B to 24% B in 10 min. 24% B; Wave Length: 254 nm; RT1 (min): 9) to afford N-(5-(5-amino-1H-pyrazol-1-yl)-1,3,4-thiadiazol-2-yl)-4-(bicyclo[1.1.1]pentan-1-ylamino)-3-methoxy-2-oxo-2H-pyran-6-carboxamide (Compound 178) (1.1 mg, 5% yield). LCMS (ES, m/z)=416.05 [M+1]+. ¹H NMR (400 MHz, Methanol-d₄) δ 7.48 (s, 1H), 7.40 (d, J=2.0 Hz, 1H), 5.51 (d, J=2.0 Hz, 1H), 3.77 (s, 3H), 2.55 (s, 1H), 2.28 (s, 6H).

### Example 53: (R)-3-methoxy-4-((1-methoxybutan-2-yl)amino)-N-(5-(5-methyl-1H-pyrazol-1-yl)-1,3,4-thiadiazol-2-yl)-2-oxo-2H-pyran-6-carboxamide (Compound 191*) and (S)-3-methoxy-4-((1-methoxybutan-2-yl)amino)-N-(5-(5-methyl-1H-pyrazol-1-yl)-1,3,4-thiadiazol-2-yl)-2-oxo-2H-pyran-6-carboxamide (Compound 192*)

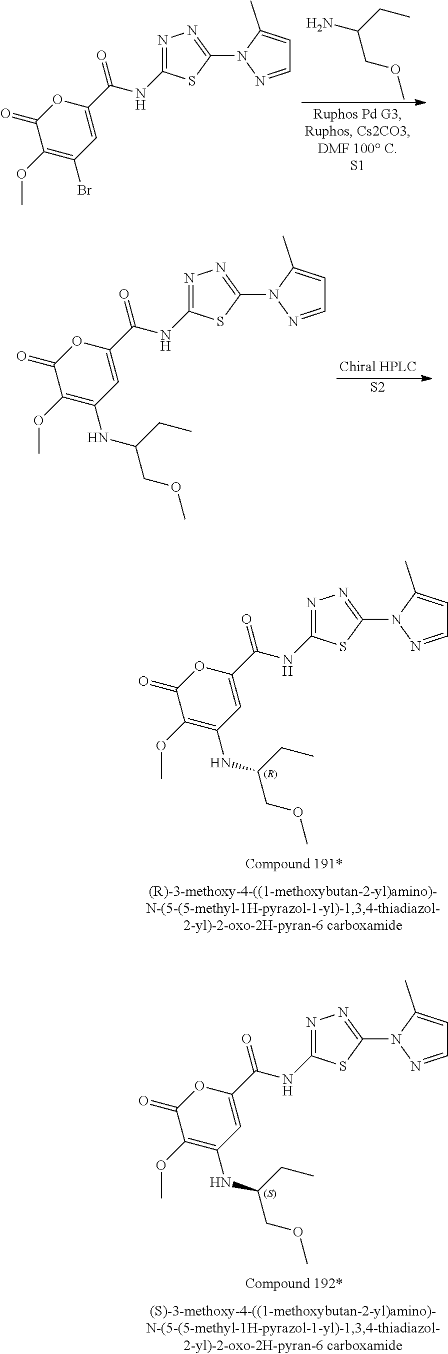

3-Methoxy-4-((1-methoxybutan-2-yl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using racemic 1-methoxybutan-2-amine as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent”. Constituent enantiomers were then separated by PREP-CHIRAL-HPLC (Column: CHIRALPAK ID, 2*25 cm, 5 μm, Mobile Phase A: methyl tert-butyl ether (MtBE) (0.1% trifluoroacetic acid (TFA)), Mobile Phase B: ethanol (EtOH):dichloromethane (DCM)=1:1; Flow rate: 20 mL/min; Gradient: 60% B to 60% B in 25 min; Wave Length: 254/220 nm; RT1 (min): 12.10; RT2 (min): 16.43; Sample Solvent: methanol (MeOH):DCM=1:1) to provide two isomers with arbitrarily assigned stereochemistry: (R)-3-methoxy-4-((1-methoxybutan-2-yl)amino)-N-(5-(5-methyl-1H-pyrazol-1-yl)-1,3,4-thiadiazol-2-yl)-2-oxo-2H-pyran-6-carboxamide (Compound 191*), first eluting peak, LCMS (ES, m/z)=435.1 [M+1]+. ¹H NMR (400 MHz, DMSO-d6) δ 7.78 (d, J=1.6 Hz, 1H), 7.41 (s, 1H), 6.44 (d, J=1.6 Hz, 1H), 3.86-3.81 (m, 1H), 3.69 (s, 3H), 3.42-3.39 (m, 2H), 3.27 (s, 3H), 2.67 (s, 3H), 1.66-1.49 (m, 2H), 0.92-0.86 (m, 3H); and (S)-3-methoxy-4-((1-methoxybutan-2-yl)amino)-N-(5-(5-methyl-1H-pyrazol-1-yl)-1,3,4-thiadiazol-2-yl)-2-oxo-2H-pyran-6-carboxamide (Compound 192*), second eluting peak, LCMS (ES, m/z)=435.0 [M+1]+, ¹H NMR (400 MHz, DMSO-d6) δ 7.78 (d, J=1.6 Hz, 1H), 7.41 (s, 1H), 6.44 (d, J=1.6 Hz, 1H), 3.86-3.81 (m, 1H), 3.69 (s, 3H), 3.42-3.39 (m, 2H), 3.27 (s, 3H), 2.67 (s, 3H), 1.66-1.49 (m, 2H), 0.92-0.86 (m, 3H).

### Example 54: (R)-3-methoxy-4-((2-methoxy-1-(2-methoxyphenyl)ethyl)amino)-N-(5-(5-methyl-1H-pyrazol-1-yl)-1,3,4-thiadiazol-2-yl)-2-oxo-2H-pyran-6-carboxamide (Compound 198*) and (S)-3-methoxy-4-((2-methoxy-1-(2-methoxyphenyl)ethyl)amino)-N-(5-(5-methyl-1H-pyrazol-1-yl)-1,3,4-thiadiazol-2-yl)-2-oxo-2H-pyran-6-carboxamide (Compound 199*)

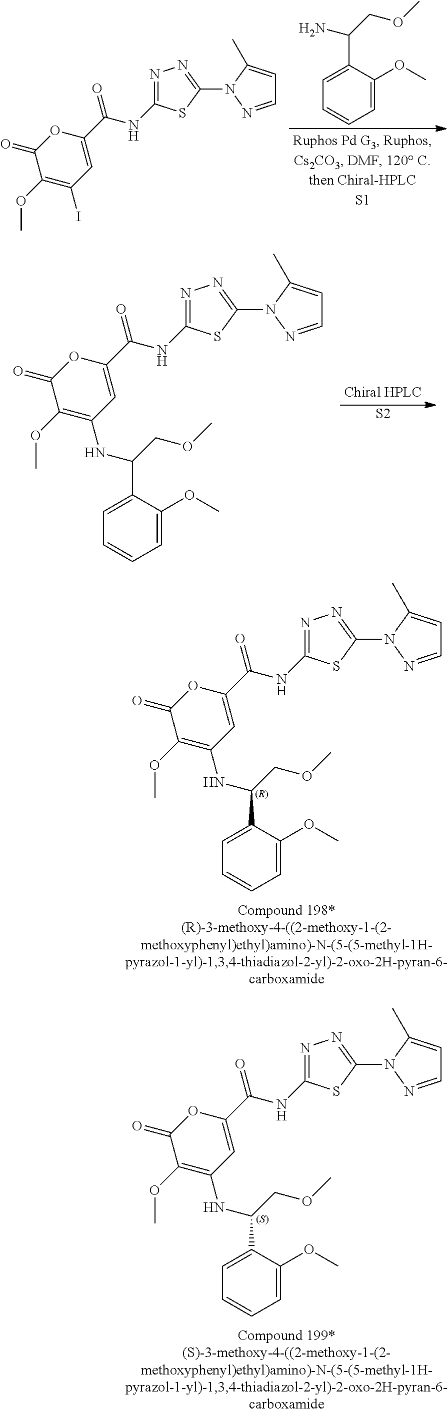

3-Methoxy-4-((2-methoxy-1-(2-methoxyphenyl)ethyl)amino)-N-(5-(5-methyl-1H-pyrazol-1-yl)-1,3,4-thiadiazol-2-yl)-2-oxo-2H-pyran-6-carboxamide was prepared according to Example 6, Step 5 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and 2-methoxy-1-(2-methoxyphenyl)ethan-1-amine as the “amine reagent”. Constituent enantiomers separated by Pre-Chiral HPLC (Column: CHIRALPAK IF, 2*25 cm, 5 μm; Mobile Phase A: Hexanes (0.1% trifluoroacetic acid (TFA)), Mobile Phase B: ethanol (EtOH):dichloromethane (DCM)=1:1; Flow rate: 20 mL/min; Gradient: 50% B to 50% B in 11.6 min; Wave Length: 220/254 nm; RT1 (min); 9.19; RT2 (min): 11.89; Sample Solvent: methanol (MeOH):DCM=1:1; Injection Volume: 0.6 mL) to provide two enantiomers with arbitrarily assigned stereochemistry: (R)-3-methoxy-4-((2-methoxy-1-(2-methoxyphenyl)ethyl)amino)-N-(5-(5-methyl-1H-pyrazol-1-yl)-1,3,4-thiadiazol-2-yl)-2-oxo-2H-pyran-6-carboxamide (Compound 198*), first eluting peak. LCMS (ES, m/z)=513.15 [M+1]+. ¹H NMR (300 MHz, DMSO-d6) δ 13.31 (br, 1H), 7.77 (s, 1H), 7.39-7.17 (m, 4H), 7.09-7.01 (m, 1H), 7.00-6.92 (m, 1H), 6.44 (s, 1H), 5.33-5.21 (m, 1H), 3.92 (s, 3H), 3.83-3.69 (m, 4H), 3.56-3.51 (m, 1H), 3.31 (s, 3H), 2.67 (s, 3H); and (5)-3-methoxy-4-((2-methoxy-1-(2-methoxyphenyl)ethyl)amino)-N-(5-(5-methyl-1H-pyrazol-1-yl)-1,3,4-thiadiazol-2-yl)-2-oxo-2H-pyran-6-carboxamide (Compound 199*), second eluting peak. LCMS (ES, m/z)=513.15 [M+1]+. ¹H NMR (300 MHz, DMSO-d6) δ 13.31 (br, 1H), 7.77 (s, 1H), 7.39-7.17 (m, 4H), 7.09-7.01 (m, 1H), 7.0-6.92 (m, 1H), 6.44 (s, 1H), 5.33-5.21 (m, 1H), 3.92 (s, 3H), 3.83-3.69 (m, 4H), 3.56-3.51 (m, 1H), 3.31 (s, 3H), 2.67 (s, 3H).

### Example 55: 4-((2-aminoethyl)amino)-3-methoxy-N-(5-(5-methyl-1H-pyrazol-1-yl)-1,3,4-thiadiazol-2-yl)-2-oxo-2H-pyran-6-carboxamide (Compound 203)

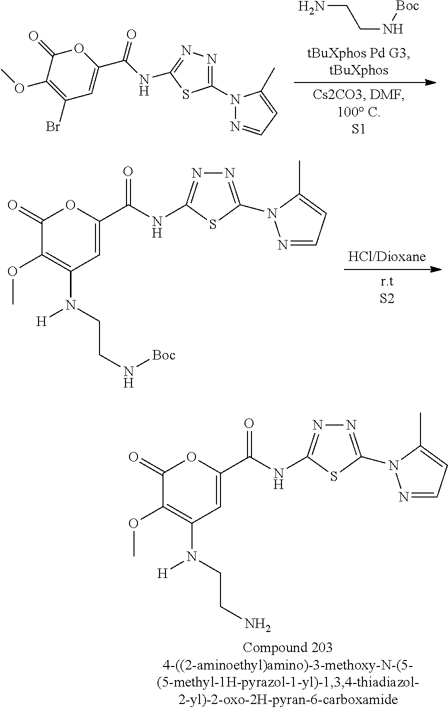

Step 1: tert-Butyl (2-((3-methoxy-6-((5-(5-methyl-1H-pyrazol-1-yl)-1,3,4-thiadiazol-2-yl)carbamoyl)-2-oxo-2H-pyran-4-yl)amino)ethyl)carbamate was prepared using 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “halo-pyrone reagent” and tert-butyl (2-aminoethyl)carbamate as the “amine reagent” according to Example 23 Step 1. LCMS (ES, m/z)=492.2 [M+1]+.

Step 2: A solution of tert-butyl (2-((3-methoxy-6-((5-(5-methyl-1H-pyrazol-1-yl)-1,3,4-thiadiazol-2-yl)carbamoyl)-2-oxo-2H-pyran-4-yl)amino)ethyl)carbamate in HCl (4 M) in 1,4-dioxane (2 mL) was stirred for 0.5 h at room temperature then concentrated under vacuum. The residue was purified by Prep-HPLC (Column: Xselect CSH C18 OBD Column 30*150 mm 5 μm; Mobile Phase A: acetonitrile (MeCN). Mobile Phase B: Water (0.05% trifluoroacetic acid (TFA)); Flow rate: 60 mL/min; Gradient: 10% B to 20% B in 10 min. 20% B; Wave Length: 254/220 nm; RT1 (min): 8.67) to afford 4-((2-aminoethyl)amino)-3-methoxy-N-(5-(5-methyl-1H-pyrazol-1-yl)-1,3,4-thiadiazol-2-yl)-2-oxo-2H-pyran-6-carboxamide 2,2,2-trifluoroacetate (Compound 203) (16 mg, 16% yield). LCMS (ES, m/z)=392.1 [M+1]+. ¹H NMR (400 MHz, DMSO-d₆) δ 7.77 (s, 1H), 7.32 (s, 1H), 6.43 (s, 1H), 3.73 (s, 3H), 3.55 (t, J=6.0 Hz, 2H), 3.01 (t, J=6.0 Hz, 2H), 2.67 (s, 3H).

### Example 56: N-(5-(5-amino-1H-pyrazol-1-yl)-1,3,4-thiadiazol-2-yl)-3-methoxy-4-((2-methoxyethyl)amino)-2-oxo-2H-pyran-6-carboxamide (Compound 205) and N-(5-(5-acetamido-1H-pyrazol-1-yl)-1,3,4-thiadiazol-2-yl)-3-methoxy-4-((2-methoxyethyl)amino)-2-oxo-2H-pyran-6-carboxamide (Compound 205-Ac)

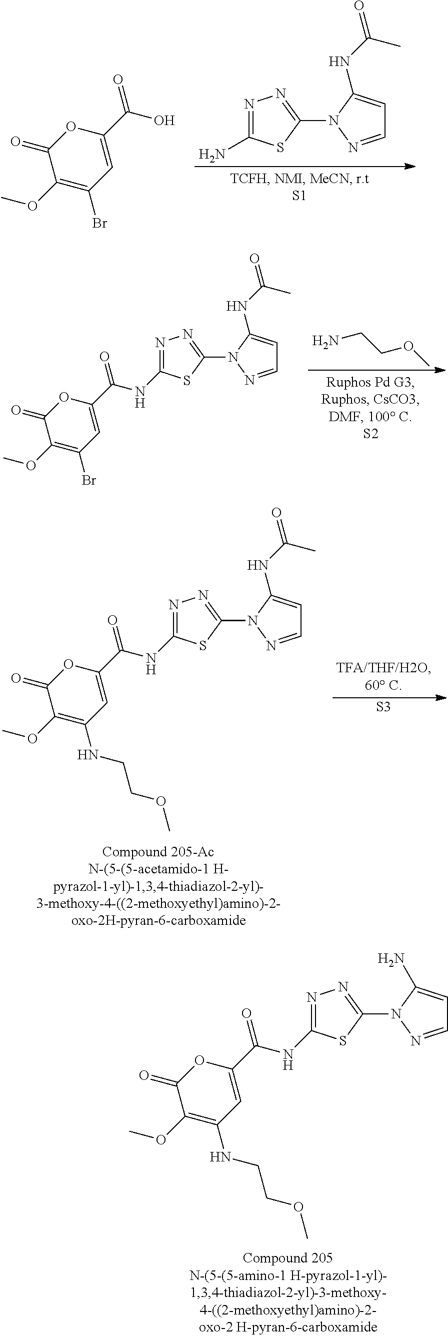

Step 1: A solution of 4-bromo-5-methoxy-6-oxopyran-2-carboxylic acid (product of Example 1, Part A, Step 4) (220 mg, 0.883 mmol, 1.00 equiv) in acetonitrile (MeCN) (3 mL) was added chloro-N,N,N′,N′-tetramethylformamidinium hexafluorophosphate (TCFH) (506 mg, 1.80 mmol, 2.04 equiv) and N-methylimidazole (NMI) (375 mg, 4.57 mmol, 5.17 equiv) was stirred for 5 min at room temperature. To the above mixture was added N-[2-(5-amino-1,3,4-thiadiazol-2-yl)pyrazol-3-yl]acetamide (product of Step 3 of Example 51) (198 mg, 0.883 mmol, 1.00 equiv). The resulting mixture was stirred for 1 h at room temperature then diluted with water (4 mL) at room temperature. The precipitated solids were collected by filtration to afford 4-bromo-N-[5-(5-acetamidopyrazol-1-yl)-1,3,4-thiadiazol-2-yl]-5-methoxy-6-oxopyran-2-arboxamide (140 mg, 35% yield). LCMS (ES, m/z)=455, 457 [M+1]+.

Step 2: A solution of 4-bromo-N-[5-(5-acetamidopyrazol-1-yl)-1,3,4-thiadiazol-2-yl]-5-methoxy-6-oxopyran-2-carboxamide (200 mg, 0.439 mmol, 1.00 equiv) in N,N-dimethylformamide (DMF) (3 mL) was added 2-Dicyclohexylphosphino-2′,6′-diisopropoxybiphenyl (RuPhos) (41 mg, 0.088 mmol, 0.20 equiv). (2-Dicyclohexylphosphino-2′,6′-diisopropoxy-1,1′-biphenyl)[2-(2′-amino-1,1′-biphenyl)]palladium(IT) methanesulfonate (RuPhos Palladacycle Gen3) (81 mg, 0.097 mmol, 0.22 equiv), Cesium carbonate (440 mg, 1.350 mmol, 3.07 equiv) and 2-methoxyethan-1-amine (80 mg, 1.065 mmol, 2.42 equiv). The mixture was stirred for 1 h at 100° C. under N₂. The mixture was then concentrated under vacuum and the residue was purified by silica gel column chromatography, eluted with dichloromethane (DCM)/methanol (MeOH) (10:1), to afford N-[5-(5-acetamidopyrazol-1-yl)-1,3,4-thiadiazol-2-yl]-5-methoxy-4-[(2-methoxyethyl)amino]-6-oxopyran-2-carboxamide (also referred to herein as N-(5-(5-acetamido-1H-pyrazol-1-yl)-1,3,4-thiadiazol-2-yl)-3-methoxy-4-((2-methoxyethyl)amino)-2-oxo-2H-pyran-6-carboxamide (Compound 205-Ac)) (60 mg, 30% yield). LCMS (ES, m/z)=450.1 [M+1]+.

Step 3: To a solution of N-[5-(5-acetamidopyrazol-1-yl)-1,3,4-thiadiazol-2-yl]-5-methoxy-4-[(2-methoxyethyl)amino]-6-oxopyran-2-carboxamide (Compound 205-Ac) (60 mg, 0.133 mmol, 1.00 equiv) in H₂O (0.6 mL) and tetrahydrofuran (THF) (0.6 mL) was slowly added trifluoroacetic acid (TFA) (1.2 mL) at 0° C. The resulting solution was stirred overnight at 50° C. then concentrated under vacuum. The crude product was purified by Prep-HPLC (Column: XBridge Prep Phenyl OBD Column, 19*150 mm, 5 μm; Mobile Phase A: Water (10 mmol/L NH₄HCO₃), Mobile Phase B: acetonitrile (MeCN); Flow rate: 60 mL/min; Gradient: 7% B to 20% B in 8 min, 20% B; Wave Length: 254 nm; RT1 (min): 7.5) to afford N-5-(5-amino-1H-pyrazol-1-yl)-3,4-thiadiazol-2-yl)-3-methoxy-4-((2-methoxyethyl)amino)-2-oxo-2H-pyran-6-carboxamide (Compound 205) (1.2 mg, 2.2% yield). LCMS (ES, m/z)=408.1 [M+1]+. ¹H NMR (400 MHz, DMSO-d6) δ 7.33 (s, 1H), 7.00 (s, 1H), 6.73-6.70 (m, 1H), 6.62-6.55 (m, 2H), 5.38 (s, 1H), 3.65 (s, 3H), 3.57-3.35 (m, 5H), 3.29-3.32 (m, 2H).

### Example 57: 4-((2-(difluoromethoxy)ethyl)amino)-3-methoxy-N-(5-(5-methyl-1H-pyrazol-1-yl)-1,3,4-thiadiazol-2-yl)-2-oxo-2H-pyran-6-carboxamide (Compound 208)

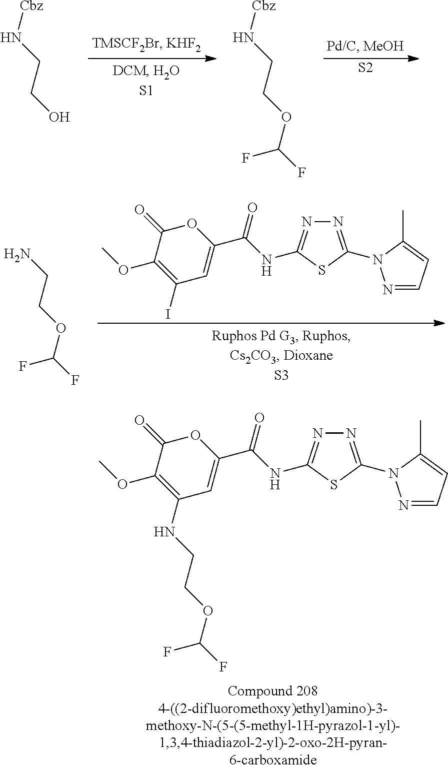 [SMILES: O=C(NCCO)OCc1ccccc1 ; O=C(NCCOC(F)F)OCc1ccccc1 ; "S2" ; NCCOC(F)F ; COc1c(I)cc(C(=O)Nc2nnc(-n3nccc3C)s2)oc1=O ; COc1c(NCCOC(F)F)cc(C(=O)Nc2nnc(-n3nccc3C)s2)oc1=O]

Step 1: To a stirred solution of benzyl N-(2-hydroxyethyl)carbamate (1.00 g, 5.12 mmol, 1.00 equiv) in dichloromethane (DCM) (6 mL) and H₂O (6 mL) was added KHF₂ (1.20 g, 15.4 mmol, 3.00 equiv) and (bromodifluoromethyl)trimethylsilane (TMS-CF₂Br) (4.16 g, 20.5 mmol, 4.00 equiv) at 0° C. The resulting mixture was stirred for 12 h at room temperature. The mixture was then diluted with DCM (100 mL) and washed with water (3×10 mL). The combined organic extracts were concentrated under reduced pressure and the crude material was purified by silica gel column chromatography, eluted with petroleum ether/ethyl acetate (PE/EtOAc) (6:1), to afford benzyl N-[2-(difluoromethoxy)ethyl]carbamate (700 mg, 56% yield). LCMS (ES, m/z)=246.1 [M+1]+. Cbz=benzyloxycarbonyl.

Step 2: Into a stirred solution of benzyl N-[2-(difluoromethoxy)ethyl]carbamate (600 mg, 2.45 mmol, 1.0) equiv) in methanol (MeOH) (30 mL) was added conc. HCl (0.2 mL) and Pd/C (100 mg, 0.940 mmol, 0.38 equiv). The resulting mixture was stirred for 4 h at room temperature under H₂ (gas). The mixture was then filtered and the filter cake was washed with MeOH (3×5 mL). The combined filtrate was concentrated under reduced pressure to provide 2-(difluoromethoxy)ethan-1-amine which was used directly without further purification. LCMS (ES, m/z)=148.55 [M+1]+.

Step 3: 4-((2-(Difluoromethoxy)ethyl)amino)-3-methoxy-N-(5-(5-methyl-1H-pyrazol-1-yl)-1,3,4-thiadiazol-2-yl)-2-oxo-2H-pyran-6-carboxamide (Compound 208) was prepared according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and 2-(difluoromethoxy)ethan-1-amine as the “amine reagent”. LCMS (ES, m/z)=443.15 [M+1]+. ¹H NMR (300 MHz, DMSO-d6) δ 13.34 (br, 1H), 7.78 (d, J=2.0 Hz, 1H), 7.39 (s, 1H), 7.16-7.12 (m, 1H), 6.95-6.43 (m, 1H), 6.42 (d, J=2.0 Hz, 1H), 3.98 (t, J=7.2 Hz, 2H), 3.70 (s, 3H), 3.62-3.57 (m, 2H), 2.68 (s, 3H).

### Example 58: 4-((trans)-2,5-bis(methoxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 209, Rac-209), 4-((2R,5R)-2,5-bis(methoxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 209a*) and 4-((2R,5R)-2,5-bis(methoxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 209b*)

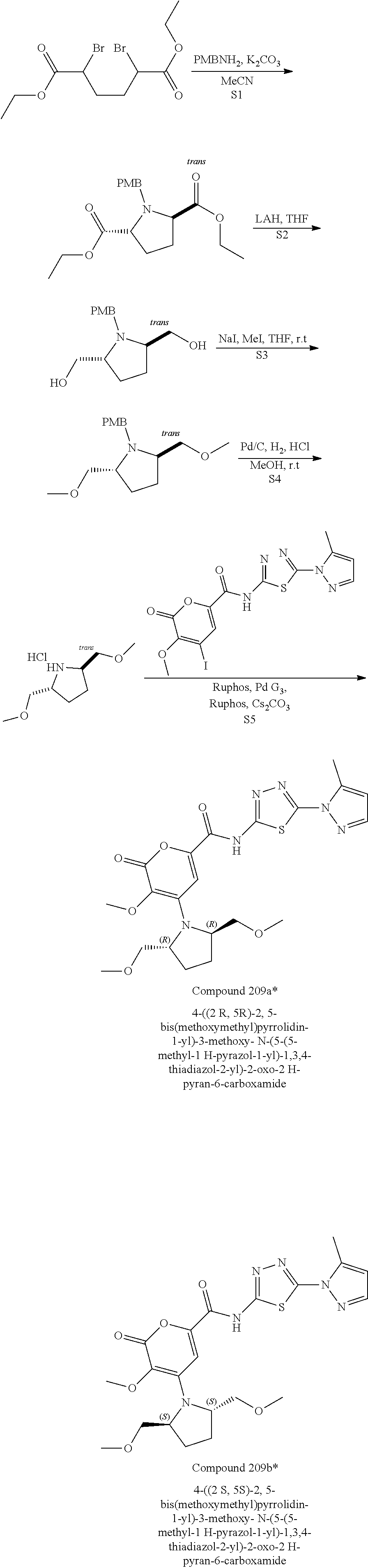

Step 1: A solution of 1,6-diethyl 2,4-dibromohexanedioate (20.0 g, 55.5 mmol, 1.00 equiv) in acetonitrile (MeCN) (70 mL) was added K₂CO₃ (9.21 g, 66.6 mmol, 1.20 equiv) and (4-methoxyphenyl)methanamine (PMB-NH₂) (7.62 g, 55.5 mmol, 1.00 equiv). The mixture was stirred for 2 h at 80° C. then diluted with water (300 mL). The mixture was then extracted with ethyl acetate (EtOAc) (3×300 mL). The combined organic layers were washed with brine (100 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (12:1), to afford trans-2,5-diethyl 1-[(4-methoxyphenyl)methyl]pyrrolidine-2,5-dicarboxylate (4.0 g, 21% yield) and cis-2,5-diethyl 1-(4-methoxybenzyl)pyrrolidine-2,5-dicarboxylate (3.0 g, 16% yield). LCMS (ES, m/z)=336.1 [M+1]+. PMB=paramethoxybenzyl.

Step 2: To a stirred solution of trans-2,5-diethyl 1-[(4-methoxyphenyl)methyl]pyrrolidine-2,5-dicarboxylate (4.0 g, 11.9 mmol, 1.00 equiv) in tetrahydrofuran (THF) (50 mL) was added LiAlH₄ (960 mg, 25.3 mmol, 2.12 equiv) dropwise at 0° C. The resulting mixture was stirred for 2 h at room temperature then quenched with water (0.96 mL) at 0° C. The mixture was then diluted with THF (50 mL) and a solution of NaOH (0.96 mL, 15% yield) in water was added at 0° C. then diluted with water (2.8 mL). The solids were filtered off and the filtrate was dried over anhydrous MgSO₄, filtered and the filtrate was concentrated under reduced pressure to provide trans-[5-(hydroxymethyl)-1-[(4-methoxyphenyl)methyl]pyrrolidin-2-yl]methanol (2.4 g, 80% yield). LCMS (ES, m/z)=252.2 [M+1]+.

Step 3: To a solution of trans-[5-(hydroxymethyl)-1-[(4-methoxyphenyl)methyl]pyrrolidin-2-yl]methanol (400 mg, 1.59 mmol, 1.0) equiv) in tetrahydrofuran (THF) (8 mL) was added and NaH (128 mg, 3.20 mmol, 2.01 equiv, 60% in mineral oil). The resulting solution was stirred for 15 min at 0° C., then methyl iodide (MeI) (1.81 g, 12.76 mmol, 8.00 equiv) was added dropwise at 0° C. The resulting mixture was stirred overnight at room temperature then quenched by the addition of water (60 mL) at room temperature and extracted with ethyl acetate (EtOAc) (3×80 mL). The combined organic layers were washed with brine (10 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (8:1), to afford trans-2,5-bis(methoxymethyl)-1-[(4-methoxyphenyl)methyl]pyrrolidine (200 mg, 45% yield). LCMS (ES, m/z)=280.3 [M+1]+.

Step 4: A solution of trans-2,5-bis(methoxymethyl)-1-[(4-methoxyphenyl)methyl]pyrrolidine (250 mg, 0.895 mmol, 1.00 equiv) in methanol (MeOH) (15 mL) was added Pd/C (100 mg, 0.940 mmol, 1.05 equiv) and conc. HCl (0.2 mL). The mixture was stirred for 1 h at room temperature under H₂ (gas). The resulting mixture was filtered, and the filter cake was washed with MeOH (6 mL). The filtrate was concentrated under reduced pressure to afford trans-2,5-bis(methoxymethyl)pyrrolidine hydrochloride (180 mg). LCMS (ES, m/z)=160.2 [M+1]+.

Step 5: 4-((2R,5R)-2,5-Bis(methoxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 209a*) and 4-((2R,5R)-2,5-bis(methoxymethyl)pyrrolidin-1-yl)-3-methoxy-N-(5-(5-methyl-1H-pyrazol-1-yl)-1,3,4-thiadiazol-2-yl)-2-oxo-2H-pyran-6-carboxamide (Compound 209b*) were prepared as a racemic mixture according to Example 1, Part C, Step 2 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as the “halo-pyrone reagent” and trans-2,5-bis(methoxymethyl)pyrrolidine hydrochloride as the “amine reagent”. LCMS (ES, m/z)=491.1 [M+1]+. ¹H NMR (400 MHz, DMSO-d6) δ 7.65 (d, J=1.6 Hz, 1H), 7.14 (s, 1H), 6.33 (d, J=1.6 Hz, 1H), 4.21-4.18 (m, 2H), 3.68 (s, 3H), 3.51 (dd, J=10.0, 4.0 Hz, 2H), 3.32 (s, 6H), 3.26 (dd, J=6.0 Hz, 3.2 Hz, 2H), 2.61 (s, 3H), 2.06-1.80 (m, 4H).

### Example 59: N-(5-(5-amino-1H-pyrazol-1-yl)-1,3,4-thiadiazol-2-yl)-4-(bicyclo[1.1.1]pentan-1-ylamino)-3-(2-methoxyethoxy)-2-oxo-2H-pyran-6-carboxamide (Compound 210) and N-(5-(5-acetamido-1H-pyrazol-1-yl)-1,3,4-thiadiazol-2-yl)-4-(bicyclo[1.1.1]pentan-1-ylamino)-3-(2-methoxyethoxy)-2-oxo-2H-pyran-6-carboxamide (Compound 210-Ac)

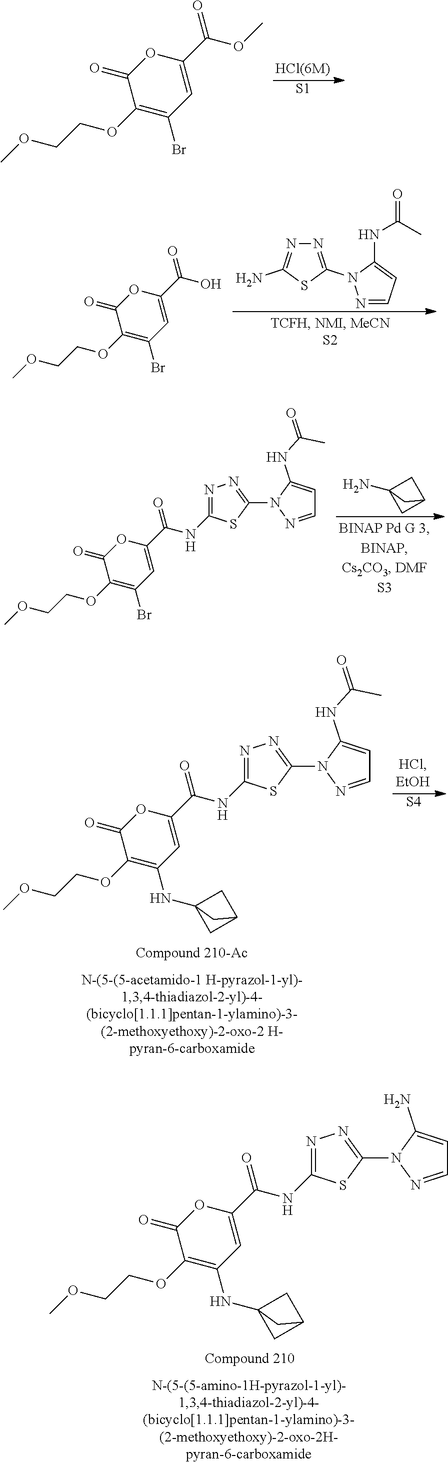

Step 1: A solution of methyl 4-bromo-5-(2-methoxyethoxy)-6-oxopyran-2-carboxylate (product of Example 15, Step 1) (1700 mg, 5.536 mmol, 1.00 equiv) in HCl (6M) (30 mL) was stirred for 3 h at 80° C. then concentrated under reduced pressure. The residue was diluted with ethyl acetate (EtOAc) (200 mL) then washed with brine (3×10 mL), and dried over anhydrous Na₂SO₄. After filtration, the filtrate was concentrated under reduced pressure to provide crude 4-bromo-5-(2-methoxyethoxy)-6-oxopyran-2-carboxylic acid, which was used in the next step directly without further purification. LCMS (ES, m/z)=293.0 [M+1]+.

Step 2: To a stirred solution of 4-bromo-5-(2-methoxyethoxy)-6-oxopyran-2-carboxylic acid (300 mg, 1.02 mmol, 1.00 equiv) and N-[2-(5-amino-1,3,4-thiadiazol-2-yl)pyrazol-3-yl]acetamide (product of Step 3 of Example 51) (253 mg, 1.13 mmol, 1.10 equiv) in acetonitrile (MeCN) (6 mL) was added chloro-N,N,N′,N′-tetramethylformamidinium hexafluorophosphate (TCFH) (575 mg, 2.05 mmol, 2.00 equiv) and N-methylimidazole (NMI) (842 mg, 10.2 mmol, 10.0 equiv) at room temperature. The resulting mixture was stirred for 1 h at room temperature then diluted with water (4 mL). The resulting mixture was filtered, and the filtrate was concentrated under reduced pressure. The residue was purified by trituration with MeCN (1 mL) and H₂O (1 mL) to afford 4-bromo-N-[5-(5-acetamidopyrazol-1-yl)-1,3,4-thiadiazol-2-yl]-5-(2-methoxyethoxy)-6-oxopyran-2-carboxamide (160 mg, 31% yield). LCMS (ES, m/z)=499.0 [M+1]+.

Step 3: To a stirred solution of 4-bromo-N-[5-(5-acetamidopyrazol-1-yl)-1,3,4-thiadiazol-2-yl]-5-(2-methoxyethoxy)-6-oxopyran-2-carboxamide (“halo-pyrone reagent”) (160 mg, 0.320 mmol, 1.00 equiv) and bicyclo[1.1.1]pentan-1-amine (“amine reagent”) (48 mg, 0.577 mmol, 1.80 equiv) in N,N-dimethylformamide (DMF) (3 mL) was added rac-BINAP-Pd-G3 ([1-(2-diphenylphosphanylnaphthalen-1-yl)naphthalen-2-yl]-diphenylphosphane methanesulfonic acid, palladium, 2-phenylaniline) (64 mg, 0.064 mmol, 0.20 equiv), (t)-2,2′-bis(diphenylphosphino)-1,1′-binaphthalene (rac-BINAP) (48 mg, 0.077 mmol, 0.24 equiv) and cesium carbonate (208 mg, 0.638 mmol, 1.99 equiv) at room temperature. The resulting mixture was stirred for 1 h at 100° C. under N₂ then purified directly by C18 reverse phase flash chromatography (acetonitrile (MeCN) in water (10 mmol/L NH₄HCO₃), 10% to 50% gradient in 10 min; detector, UV 254 nm) to provide N-(5-(5-acetamido-1H-pyrazol-1-yl)-1,3,4-thiadiazol-2-yl)-4-(bicyclo[1.1.1]pentan-1-ylamino)-3-(2-methoxyethoxy)-2-oxo-2H-pyran-6-carboxamide (Compound 210-Ac) (55 mg, 34% yield). LCMS (ES, mli)=502.50 [M+1]+.

Step 4: A stirred solution of N-(5-(5-acetamido-1H-pyrazol-1-yl)-1,3,4-thiadiazol-2-yl)-4-(bicyclo[1.1.1]pentan-1-ylamino)-3-(2-methoxyethoxy)-2-oxo-2H-pyran-6-carboxamide (Compound 210-Ac) (80 mg, 0.160 mmol, 1.00 equiv) in ethanol (EtOH) (1 mL) and HCl (2 mL, 6 M) was stirred for 12 h at room temperature. The mixture was then concentrated under reduced pressure and the crude product purified by Prep-HPLC (Column: XBridge Prep OBD C18 Column, 30*150 mm, 5 μm; Mobile Phase A: Water (10 mmol/L NH₄CO₃), Mobile Phase B: acetonitrile (MeCN); Flow rate: 60 mL/min; Gradient: 20% B to 30% B in 10 min, 30% B; Wave Length: 254 nm; RT (min): 8.5) to afford N-[5-(5-aminopyrazol-1-yl)-1,3,4-thiadiazol-2-yl]-4-{bicyclo[1.1.1]pentan-1-ylamino}-5-(2-methoxyethoxy)-6-oxopyran-2-carboxamide (Compound 210) (21 mg, 28% yield). LCMS (ES, m/z)=460.15 [M+1]+. ¹H NMR (300 MHz, DMSO-d6) S 7.41 (s, 1H), 7.26 (s, 1H), 7.03 (s, 1H), 6.62 (s, 2H), 5.42-5.41 (m, 1H), 4.00 (t, J=5.2 Hz, 2H), 3.55 (t, J=5.2 Hz, 2H), 3.33 (s, 3H), 2.56 (s, 1H), 2.17 (s, 6H).

### Example 60: 3-methoxy-4-((2S,3R)-3-methoxy-2-(methoxymethyl)pyrrolidin-1-yl)-N-(5-(5-methyl-1H-pyrazol-1-yl)-1,3,4-thiadiazol-2-yl)-2-oxo-2H-pyran-6-carboxamide (Compound 211)

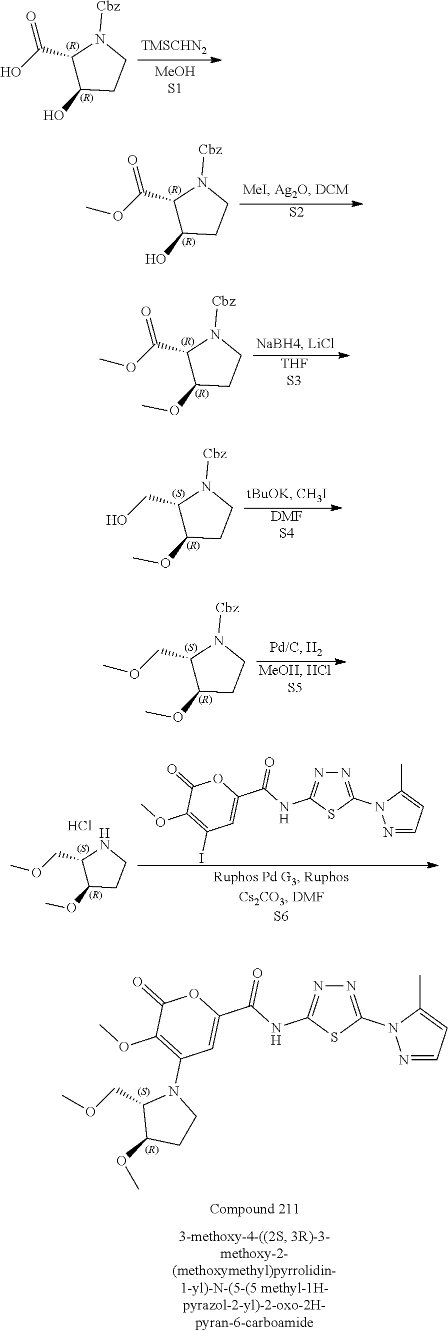

Step 1: To a solution of (2R,3R)-1-[(benzyloxy)carbonyl]-3-hydroxypyrrolidine-2-carboxylic acid (1000 mg, 3.770 mmol, 1.00 equiv) in methanol (MeOH) (10 mL) was added trimethylsilyldiazomethane (TMSCHN₂) (20 mL, 0.600 mmol) at room temperature. The resulting mixture was stirred for 2 h at room temperature then concentrated under vacuum to afford 1-benzyl 2-methyl (2R,3R)-3-hydroxypyrrolidine-1,2-dicarboxylate (1.11 g), which was used in the next step without further purification. LCMS (ES, m/z)=280 [M+1]+. Cbz=benzyloxycarbonyl.

Step 2: To a solution of 1-benzyl 2-methyl (2R,3R)-3-hydroxypyrrolidine-1,2-dicarboxylate (1.11 g, 3.97 mmol, 1.00 equiv) in dichloromethane (DCM) (20 mL) was added was added methyl iodide (MeI) (1.69 g, 11.9 mmol, 3.00 equiv) and Ag₂O (1.84 g, 7.95 mmol, 2.00 equiv). The resulting mixture was stirred overnight at room temperature in the dark. The resulting mixture was filtered, and the filtrate was concentrated under reduced pressure to afford 1-benzyl 2-methyl (2R,3R)-3-methoxypyrrolidine-1,2-dicarboxylate (1.0 g, 86% yield). LCMS (ES, m/z)=294 [M+1]+.

Step 3: To a solution of 1-benzyl 2-methyl (2R,3R)-3-methoxypyrrolidine-1,2-dicarboxylate (500 mg, 1.70 mmol, 1.00 equiv) in tetrahydrofuran (THF) (6 mL) was added NaBH₄ (258 mg, 6.82 mmol, 4.00 equiv) and LiCl (285 mg, 6.72 mmol, 3.94 equiv). The resulting solution was stirred overnight at room temperature. The reaction was quenched with water (10 mL) and extracted with ethyl acetate (EtOAc) (3×20 mL). The combined organic layers were dried over anhydrous Na₂SO₄. After filtration, the filtrate was concentrated under reduced pressure to afford benzyl (2S,3R)-2-(hydroxymethyl)-3-methoxypyrrolidine-1-carboxylate (400 mg, 88% yield). LCMS (ES, m/z)=266 [M+1]+.

Step 4: To a solution of benzyl (2S,3R)-2-(hydroxymethyl)-3-methoxypyrrolidine-1-carboxylate (400 mg, 1.51 mmol, 1.00 equiv) and potassium tert-butoxide (t-BuOK) (860 mg, 7.66 mmol, 5.08 equiv) in N,N-dimethylformamide (DMF) (15 mL) was added in methyl iodide (MeI) (1.12 g, 7.89 mmol, 5.23 equiv) at 0° C. And then the resulting mixture was stirred for 1 h at room temperature. The mixture was then diluted in ethyl acetate (EtOAc) (100 mL), washed with water (3×20 mL). The organic layer was then concentrated under vacuum to afford benzyl (2S,3R)-3-methoxy-2-(methoxymethyl)pyrrolidine-1-carboxylate (300 mg, 71% yield). LCMS (ES, m/z)=280 [M+1]+.

Step 5: A solution of benzyl (2S,3R)-3-methoxy-2-(methoxymethyl)pyrrolidine-1-carboxylate (300 mg, 1.07 mmol, 1.00 equiv) in methanol (MeOH) (15 mL) was added HCl (5 uL) and Pd/C (400 mg). The mixture was stirred for 2 h at room temperature under H₂ (gas). The mixture was filtered, and the filtrate was concentrated under reduced pressure to afford (2S,3R)-3-methoxy-2-(methoxymethyl)pyrrolidine (120 mg, 77% yield). LCMS (ES, m/z)=146 [M+1]+.

Step 6: 3-Methoxy-4-((2S,3R)-3-methoxy-2-(methoxymethyl)pyrrolidin-1-yl)-N-(5-(5-methyl-1H-pyrazol-1-yl)-1,3,4-thiadiazol-2-yl)-2-oxo-2H-pyran-6-carboxamide (Compound 211) was prepared according to Example 6, Step 5 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and (2S,3R)-3-methoxy-2-(methoxymethyl)pyrrolidine hydrochloride as the “amine reagent”. LCMS (ES, m/z)=477.15 [M+1]+. ¹H NMR (400 MHz, DMSO-d6) δ 13.50 (br, 1H), 7.76 (s, 1H), 7.25 (s, 1H), 6.43 (s, 1H), 4.36 (d, J=2.4 Hz, 1H), 3.91-3.83 (m, 2H), 3.72 (s, 3H), 3.58-3.51 (m, 1H), 3.47-3.43 (m, 2H), 3.32 (s, 6H), 2.83 (s, 3H), 2.33-2.13 (m, 2H).

### Example 61: 4-(((1S,2R)-2-(difluoromethoxy)cyclopentyl)amino)-3-methoxy-N-(5-(5-methyl-1H-pyrazol-1-yl)-1,3,4-thiadiazol-2-yl)-2-oxo-2H-pyran-6-carboxamide (Compound 212)

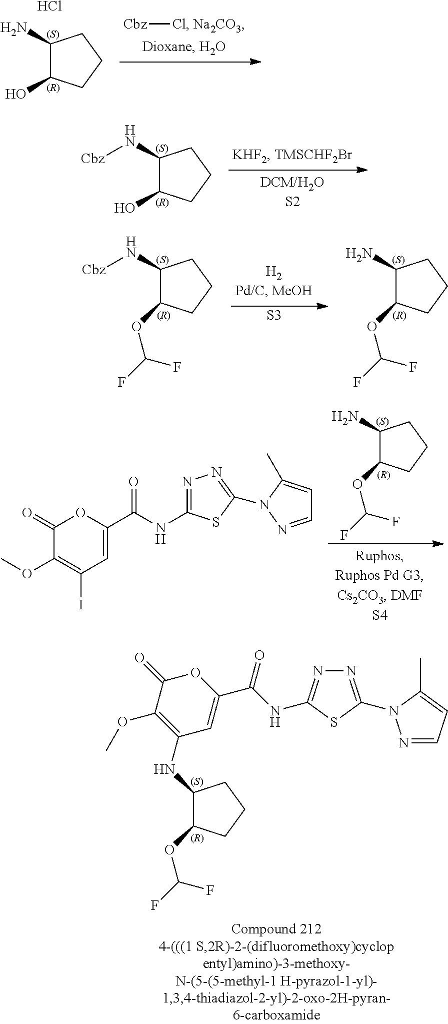

Step 1: To a solution of (1R,2S)-2-aminocyclopentan-1-ol hydrochloride (300 mg, 2.18 mmol, 1.00 equiv) in dioxane (0.6 mL) and H₂O (3 mL) was added benzyl chloroformate (Cbz-Cl) (558 mg, 3.27 mmol, 1.50 equiv) and Na₂CO₃ (462 mg, 4.36 mmol, 2.00 equiv) at 0° C. The resulting mixture was stirred overnight at room temperature. The reaction was quenched by the addition of water (10 mL), and extracted with ethyl acetate (EtOAc) (3×20 mL). The combined organic layers were washed with brine (10 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (4:1), to afford benzyl N-[(1S,2R)-2-hydroxycyclopentyl]-carbamate (450 mg, 79% yield). Cbz=benzyloxycarbonyl.

Step 2: To a solution of benzyl N-[(1S,2R)-2-hydroxycyclopentyl]carbamate (400 mg, 1.70 mmol, 1.00 equiv) in H₂O (14 mL) and dichloromethane (DCM) (14 mL) was added (bromodifluoromethyl)trimethyl-silane (TMS-CF₂Br) (4.00 g, 19.7 mmol, 11.6 equiv) and KHF₂ (1.06 g, 13.6 mmol, 8.00 equiv) at room temperature. The resulting mixture was stirred for 17 h at room temperature. The mixture was then diluted with DCM (100 mL). The organic layer was washed with brine (20 mL), and dried over anhydrous Na₂SO₄. After filtration, the filtrate was concentrated under reduced pressure and the residue was purified by silica gel column chromatography, eluted with petroleum ether/ethyl acetate (PE/EtOAc) (1:1), to afford benzyl N-[(1S,2R)-2-(difluoromethoxy) cyclopentyl]carbamate (230 mg, 43% yield). LCMS (ES, m/z)=286.1 [M+1]+.

Step 3: A solution of benzyl N-[(1S,2R)-2-(difluoromethoxy)cyclopentyl]carbamate (230 mg, 0.806 mmol, 1.00 equiv) in methanol (MeOH) (1 mL) was added Pd/C (30 mg). The mixture was hydrogenated at room temperature for 2 h under hydrogen atmosphere using a hydrogen balloon, then filtered through a Celite pad and concentrated under reduced pressure to provide (1S,2R)-2-(difluoromethoxy)cyclopentan-1-amine (100 mg, 74% yield). LCMS (ES, m/z)=152.1 [M+1]+.

Step 4: 4-(((1S,2R)-2-(Difluoromethoxy)cyclopentyl)amino)-3-methoxy-N-(5-(5-methyl-1H-pyrazol-1-yl)-1,3,4-thiadiazol-2-yl)-2-oxo-2H-pyran-6-carboxamide (Compound 212) was prepared according to Example 6, Step 5 using 4-iodo-3-methoxy-N-(5-(5-methyl-1H-pyrazol-1-yl)-1,3,4-thiadiazol-2-yl)-2-oxo-2H-pyran-6-carboxamide (product of Step 4 of Example 6) as “halo-pyrone reagent” and (1S,2R)-2-(difluoromethoxy)cyclopentan-1-amine as the “amine reagent”. LCMS (ES, m/z)=483.05 [M+1]+. ¹H NMR (400 MHz, DMSO-4) δ 7.66 (s, 1H), 7.18 (s, 1H), 6.89-6.55 (m, 1H), 6.34 (s, 1H), 6.14-6.08 (m, 1H), 4.62-4.57 (m, 1H), 4.27-4.12 (m, 1H), 3.67 (s, 3H), 2.68 (s, 3H), 2.05-1.95 (m, 2H), 1.90-1.76 (m, 3H), 1.61-1.59 (m, 1H).

### Example 62: 3-methoxy-4-(((1S,2R)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 214*) and 3-methoxy-4-(((1R,2S)-2-methoxycyclopentyl)amino)-N-(5-(5-methyl-1H-pyrazol-1-yl)-1,3,4-thiadiazol-2-yl)-2-oxo-2H-pyran-6-carboxamide (Compound 215*)

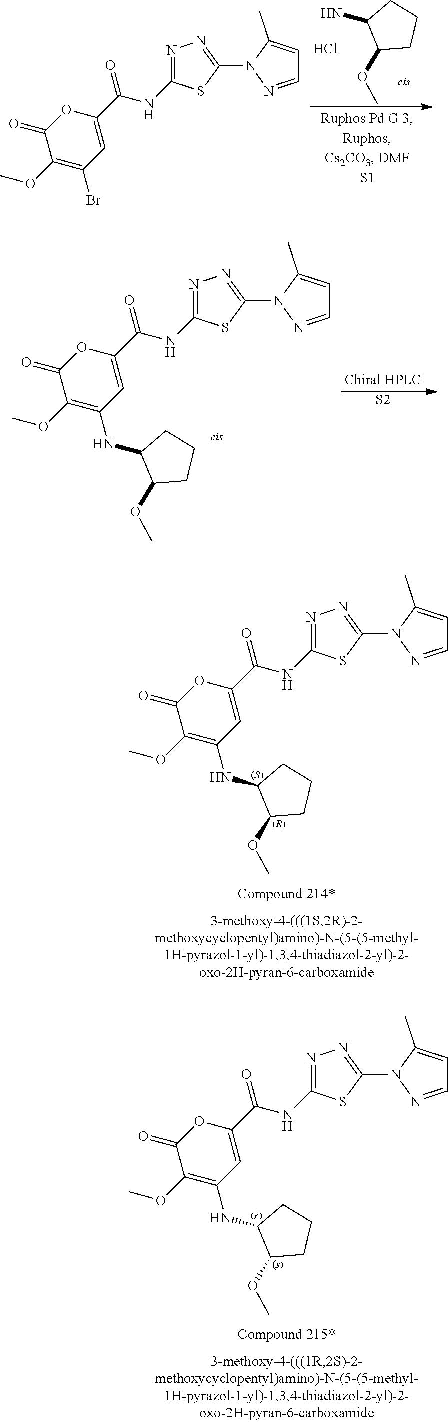

Racemic 3-methoxy-4-(((1,2-cis)-2-methoxycyclopentyl)amino)-N-(5-(5-methyl-1H-pyrazol-1-yl)-1,3,4-thiadiazol-2-yl)-2-oxo-2H-pyran-6-carboxamide was prepared according to Example 1, Part C, Step 2 using cis-2-methoxycyclopentan-1-amine hydrochloride as the “amine reagent” and 4-bromo-5-methoxy-N-[5-(5-methylpyrazol-1-yl)-1,3,4-thiadiazol-2-yl]-6-oxopyran-2-carboxamide (product of Example 1, Part C, Step 1) as the “hako-pyrone reagent”. Constituent enantiomers were separated by chiral prep-HPLC (Column: CHIRALPAK IG-3, 4.6*50 mm, 3 um; Mobile Phase: Hexanes (0.1% trifluoroacetic acid (TFA)):(ethanol (EtOH):dichloromethane (DCM)=1:1)=30:70; Flow rate: 1 mL/min) to provide two enantiomers with arbitrarily assigned stereochemistry: 3-methoxy-4-(((1S,2R)-2-methoxycyclopentyl)amino)-N-5-(5-methyl-1H-pyrazol-1-yl)-1,3,4-thiadiazol-2-yl)-2-oxo-2H-pyran-6-carboxamide (Compound 214*), first eluting peak, LCMS (ES, m/z)=447.1 [M+1]+, ¹HNMR (400 MHz, DMSO-d6) δ 13.33 (br, 1H), 7.79 (d, J=1.6 Hz, 1H), 7.42 (s, 1H), 6.45 (d, J=1.6 Hz, 1H), 6.17 (br, 1H), 4.14-4.01 (m, 1H), 3.80-3.75 (m, 1H), 3.73 (s, 3H), 3.27 (s, 3H), 2.68 (s, 3H), 2.06-1.95 (m, 1H), 1.85-1.50 (m, 5H); and 3-methoxy-4-(((1R,2S)-2-methoxycyclopentyl)amino)-N-(5-(5-methyl-1-H-pyrazol-1-yl)-1,3,4-thiadiazol-2-yl)-2-oxo-2H-pyran-6-carboxamide (Compound 215*), second eluting peak, LCMS (ES, m/z)=447.1 [M+1]+, ¹HNMR (400 MHz, DMSO-d6) δ 13.33 (br, 1H), 7.79 (d, J=1.6 Hz, 1H), 7.42 (s, 1H), 6.45 (d, J=1.6 Hz, 1H), 6.17 (br, 1H), 4.14-4.01 (m, 1H), 3.80-3.75 (m, 1H), 3.73 (s, 3H), 3.27 (s, 3H), 2.68 (s, 3H), 2.06-1.95 (m, 1H), 1.85-1.50 (m, 5H).

### Example 63: 4-((2-oxaspiro[3.3]heptan-5-yl)amino)-3-methoxy-N-(5-(5-methyl-1H-pyrazol-1-yl)-1,3,4-thiadiazol-2-yl)-2-oxo-2H-pyran-6-carboxamide (Compound 56, Rac-56), (S)-4-((2-oxaspiro[3.3]heptan-5-yl)amino)-3-methoxy-N-(5-(5-methyl-1H-pyrazol-1-yl)-1,3,4-thiadiazol-2-yl)-2-oxo-2H-pyran-6-carboxamide (Compound 56a*), and (R)-4-((2-oxaspiro[3.3]heptan-5-yl)amino)-3-methoxy-N-(5-(5-methyl-1H-pyrazol-1-yl)-1,3,4-thiadiazol-2-yl)-2-oxo-2H-pyran-6-carboxamide (Compound 56b*)

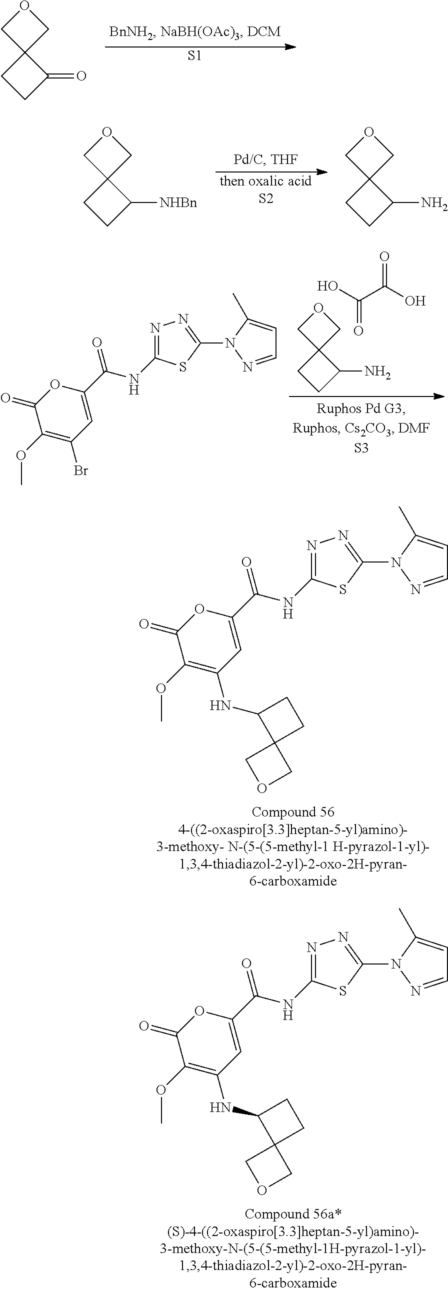

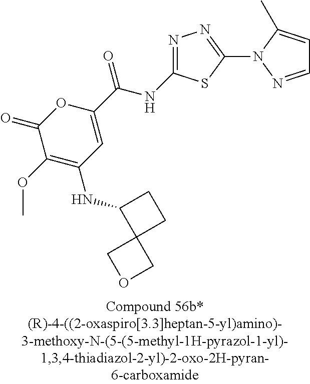 [SMILES: COc1c(N[C@@H]2CCC23COC3)cc(C(=O)Nc2nnc(-n3nccc3C)s2)oc1=O]

Step 1: To a stirred solution of 2-oxaspiro[3.3]heptan-5-one (300 mg, 2.68 mmol, 1.00 equiv) and benzylamine (BnNH₂) (570 mg, 5.32 mmol, 1.99 equiv) in dichloromethane (DCM) (5 mL) was added sodium triacetoxyborohydride (NaBH(OAc)₃) (1140 mg, 5.38 mmol, 2.01 equiv) at room temperature. The resulting mixture was stirred overnight at room temperature then quenched with water at room temperature. The mixture was then extracted with ethyl acetate (EtOAc) (3×20 mL) and the combined organic layers were washed with water (3×5 mL), and dried over anhydrous Na₂SO₄. After filtration, the filtrate was concentrated under reduced pressure and the residue was purified by C18 reverse phase flash chromatography (acetonitrile (MeCN) in water, 0% to 100% gradient in 10 min; detector, UV 254 nm) to provide N-benzyl-2-oxaspiro[3.3]heptan-5-amine (350 mg, 64% yield). LCMS (ES, m/z)=204 [M+1]+.

Step 2: To a stirred solution of N-benzyl-2-oxaspiro[3.3]heptan-5-amine (300 mg, 1.48 mmol, 1.00 equiv) in tetrahydrofuran (THF) (5 mL) was added Pd/C (00 mg, 0.94 mmol, 0.64 equiv) at room temperature under H₂ (gas). The resulting mixture was stirred overnight at room temperature under H₂ (gas). The mixture was then filtered, and the filter cake was washed with THF (3×5 mL). To the filtrate was added oxalic acid (140 mg, 1.55 mmol, 1.05 equiv) and then stirred for 0.5 h. The resulting solids were collected by filtration and washed with THF (2×1 mL) to provide 2-oxaspiro[3.3]heptan-5-amine oxalate (80 mg, 48% yield). LCMS (ES, m/z)=114 [M+1]+.

Step 3: 4-((2-Oxaspiro[3.3]heptan-5-yl)amino)-3-methoxy-N-(5-(5-methyl-1H-pyrazol-1-yl)-1,3,4-thiadiazol-2-yl)-2-oxo-2H-pyran-6-carboxamide (Compound 56) was prepared as a racemic mixture (comprising Compounds 56a* and 56b*) according to Example 1, Part C, Step 2 using 2-oxaspiro[3.3]heptan-5-amine oxalate as the “amine reagent” and 4-bromo-5-methoxy-N-[5-(5-methylpyrazol-1-vi)-1,3,4-thiadiazol-2-yl]-6-oxopyran-2-carboxamide (product of Example 1, Part C, Step 1) as the “halo-pyrone reagent”. LCMS (ES, m/z)=445.15 [M+1]+. ¹H NMR (400 MHz, DMSO-d6) δ 7.62 (d, J=1.6 Hz, 1H), 7.21 (s, 1H), 7.02-6.98 (m, 1H), 6.32 (d, J=1.6 Hz, 1H), 4.75 (d, J=6.5 Hz, 1H), 4.58 (d, J=6.9 Hz, 1H), 4.45 (dd, J=12.5, 6.8 Hz, 2H), 4.30 (q, J=8.5 Hz, 1H), 3.70 (s, 3H), 2.60 (s, 3H), 2.08-1.92 (m, 4H).

### Example 64: 1-(1,3-dimethoxypropan-2-yl)-N-(5-(5-methyl-1H-pyrazol-1-yl)-1,3,4-thiadiazol-2-yl)-5-oxo-1,2,3,5-tetrahydropyrano[3,4-b][1,4]oxazine-7-carboxamide (Compound 221)

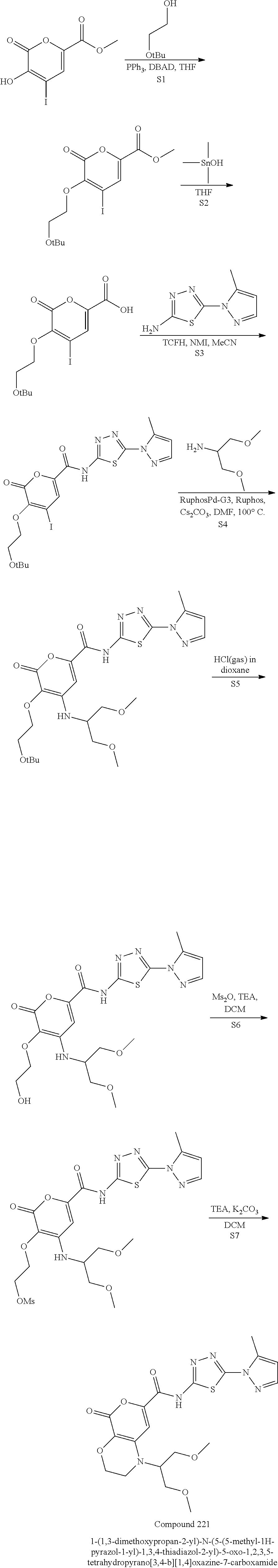

Step 1: To a stirred mixture of methyl 5-hydroxy-4-iodo-6-oxopyran-2-carboxylate (product of Step 1, Example 6) (1.00 g, 3.38 mmol, 1 equiv) and triphenyl phosphine (PPh₃) (1.33 g, 5.06 mmol, 1.5 equiv) in tetrahydrofuran (THF) (15 mL) was added di-tert-butyl azodicarboxylate (DBAD) (1.17 g, 5.06 mmol, 1.5 equiv) in portions at 0° C. The resulting mixture was stirred for 10 min at 0° C. To the above mixture was added 2-(t-butoxy)ethanol (478 mg, 4.04 mmol, 1.2 equiv) in portions at 0° C. The resulting mixture was stirred for additional overnight at room temperature. The reaction was quenched by the addition of water (150 mL) at room temperature. The resulting mixture was extracted with ethyl acetate (EtOAc) (3×30 mL). The combined organic layers were washed with NaCl (3 mL) and dried over anhydrous Na₂SO₄. After filtration, the filtrate was concentrated under reduced pressure. The residue was purified by silica gel column chromatography, eluting with petroleum ether/ethyl acetate (PE/EtOAc) (10:1), to afford methyl 5-[2-(tert-butoxy)ethoxy]-4-iodo-6-oxopyran-2-carboxylate (1 g, 74% yield).

Step 2: Into a 8 mL vial was added methyl 5-[2-(tert-butoxy)ethoxy]4-iodo-6-oxopyran-2-carboxylate (380 mg, 0.959 mmol, 1 equiv) in tetrahydrofuran (THF) (2 mL) and trimethyltin hydroxide (180 mg, 0.995 mmol, 1 equiv) at room temperature. The resulting mixture was stirred for 16 h at room temperature. The resulting mixture was concentrated under reduced pressure. The residue was purified by reversed-phase flash chromatography (C18 silica gel; mobile phase, acetonitrile (MeCN) in water, 10% to 20% gradient in 10 min; detector, UV 254 nm) to provide 5-[2-(tert-butoxy)ethoxy]-4-iodo-6-oxopyran-2-carboxylic acid (370 mg, 96% yield).

Step 3: Into a 40 mL vial was added 5-[2-(tert-butoxy)ethoxy]-4-iodo-6-oxopyran-2-carboxylic acid (370 mg, 0.968 mmol, 1 equiv), acetonitrile (MeCN) (5 mL), chloro-N,N,N′,N′-tetramethylformamidinium hexafluorophosphate (TCFH) (461 mg, 1.643 mmol, 1.7 equiv), N-methylimidazole (NMI) (238 mg, 2.90 mmol, 3 equiv) and 5-(5-methylpyrazol-1-yl)-1,3,4-thiadiazol-2-amine (product of Example 1, Part B, Step 4) (180 mg, 0.993 mmol, 1 equiv) at room temperature. The resulting mixture was stirred for 1 h at room temperature. The precipitated solids were collected by filtration, washed with acetonitrile (MeCN) (3×2 mL), and dried by lyophilization to provide 5-[2-(tert-butoxy)ethoxy]-4-iodo-N-[5-(5-methylpyrazol-1-yl)-1,3,4-thiadiazol-2-yl]-6-oxopyran-2-carboxamide (230 mg, 41% yield).

Step 4: Into a 40 mL vial containing 5-[2-(tert-butoxy)ethoxy]-4-iodo-N-[5-(5-methylpyrazol-1-yl)-1,3,4-thiadiazol-2-yl]-6-oxopyran-2-carboxamide (220 mg, 0.403 mmol, 1 equiv) and 1,3-dimethoxypropan-2-amine as the “amine reagent” (110 mg, 0.923 mmol, 2.3 equiv) in N,N-dimethylformamide (DMF) (3.5 mL) was added (2-dicyclohexylphosphino-2′,6′-diisopropoxy-1,1′-biphenyl)[2-(2′-amino-1,1′-biphenyl)]palladium(II) methanesulfonate (RuPhos Palladacycle Gen3) (67 mg, 0.080 mmol, 0.2 equiv), 2-dicyclohexylphosphino-2′,6′-diisopropoxybiphenyl (RuPhos) (38 mg, 0.081 mmol, 0.2 equiv) and Cs₂CO₃ (396 mg, 1.215 mmol, 3 equiv) at room temperature. The resulting mixture was stirred for 2 h at 100° C. under nitrogen atmosphere. The reaction was quenched with water at room temperature. The aqueous layer was extracted with ethyl acetate (EtOAc) (3×5 mL). The resulting mixture was concentrated under reduced pressure. The residue was purified by reversed-phase flash chromatography (C18 silica gel: mobile phase, acetonitrile (MeCN) in water, 10% to 50% gradient in 10 min; detector, UV 254 nm) to provide 5-[2-(tert-butoxy)ethoxy]-4-[(1,3-dimethoxypropan-2-yl)amino]-N-[5-(5-methylpyrazol-1-yl)-1,3,4-thiadiazol-2-yl]-6-oxopyran-2-carboxamide (150 mg, 62% yield).

Step 5: Into a 50 mL round-bottom flask were added 5-[2-(tert-butoxy)ethoxy]-4-[(1,3-dimethoxypropan-2-yl)amino]-N-[5-(5-methylpyrazol-1-yl)-1,3,4-thiadiazol-2-yl]-6-oxopyran-2-carboxamide (150 mg, 0.280 mmol, 1 equiv) and HCl (gas) in 1,4-dioxane (5 mL) at room temperature. The resulting mixture was stirred for 16 h at room temperature. The resulting mixture was concentrated under reduced pressure. This resulted in 4-[(1,3-dimethoxypropan-2-yl)amino]-5-(2-hydroxyethoxy)-N-[5-(5-methylpyrazol-1-yl)-1,3,4-thiadiazol-2-yl]-6-oxopyran-2-carboxamide (145 mg, 97% yield), which was used in the next step directly without further purification.

Step 6: Into a 50 mL round-bottom flask was added 4-[(1,3-dimethoxypropan-2-yl)amino]-5-(2-hydroxyethoxy)-N-[5-(5-methylpyrazol-1-yl)-1,3,4-thiadiazol-2-yl]-6-oxopyran-2-carboxamide (150 mg, 0.312 mmol, 1 equiv), dichloromethane (DCM) (6 mL) and triethylamine (TEA) (95 mg, 0.94 mmol, 3 equiv) at room temperature. To the above mixture was added methanesulfonyl methanesulfonate (135 mg, 0.775 mmol, 2.5 equiv) in portions at 0° C. The resulting mixture was stirred overnight at room temperature, and then quenched with water at room temperature. The aqueous layer was extracted with DCM (3×10 mL), the organic layer concentrated under reduced pressure, to provide a residue which was purified by silica gel column chromatography, eluting with petroleum ether/ethyl acetate (PE/EtOAc) (1:1), to provide 2-({4-[(1,3-dimethoxypropan-2-yl)amino]-6-{[5-(5-methylpyrazol-1-yl)-1,3,4-thiadiazol-2-yl]carbamoyl}-2-oxopyran-3-yl}oxy)ethyl methanesulfonate (80 mg, 41% yield).

Step 7: Into a 40 mL vial was added 2-((4-[(1,3-dimethoxypropan-2-yl)amino]-6-{[5-(5-methylpyrazol-1-yl)-1,3,4-thiadiazol-2-yl]carbamoyl}-2-oxopyran-3-yl)oxy)ethyl methanesulfonate (70 mg, 0.12 mmol, 1 equiv), dichloromethane (DCM) (5 mL), triethylamine (TEA) (100 mg, 0.988 mmol, 8 equiv) and K₂CO₃ (100 mg, 0.724 mmol, 6 equiv) at room temperature. The resulting mixture was stirred for 16 h at 40° C., and then quenched with water at room temperature. The aqueous layer was extracted with DCM (3×5 mL), the organic layer concentrated under reduced pressure, and the residue was purified by reversed-phase flash chromatography (C18 silica gel, mobile phase, acetonitrile (MeCN) in water, 10% to 50% gradient in 10 min; detector, UV 254 nm) to provide a crude product, which was further purified by Chiral-Prep-HPLC (XBridge Prep Phenyl OBD Column, 19*250 mm, 5 μm; mobile phase, water (0.05% trifluoroacetic acid (TFA)) and MeCN (35% MeCN up to 45% in 10 min); Detector, UV 254 nm) to provide 1-(1,3-dimethoxypropan-2-yl)-N-(5-(5-methyl-1H-pyrazol-1-yl)-5-oxo-1,2,3,5-tetrahydropyrano[3,4-b][1,4]oxazine-7-carboxamide (5.5 mg, 9% yield). LCMS (ES, m/z)=363.1 [M+1]+. ¹H NMR (400 MHz, DMSO-d₆) δ 7.73 (d, J=2.9 Hz, 1H), 7.53 (d, J=3.4 Hz, 1H), 6.41 (s, 1H), 4.45-4.32 (m, 2H), 4.07 (d, J=4.2 Hz, 2H), 3.59-3.50 (m, 2H), 3.49-3.35 (m, 4H), 3.24 (d, J=2.2 Hz, 6H), 2.65 (d, J=2.5 Hz, 3H).

Compounds provided in below Table A were synthesized, or may be synthesized, following the Procedures as described, with reference to the Examples. LC-MS data is provided for each compound synthesized. Dashed lines (--) indicate no data available. For purposes of the Examples and the Assay section, “Rac-X” signifies a mixture of stereoisomers: Compounds “Xa” and “Xb”. Structures of the compounds listed in Table A are also provided in Table 1 or Table 2.

TABLE A
Additional compounds

| | | LCMS | |
|---|---|---|---|
| Comp'd # | Name | [M + 1]+ | Procedure |
| 1 | (R)-3-methoxy-4-(3- | 447.1 | Prepared according to Example 1, |
| | (methoxymethyl)pyrrolidin-1-yl)-N- | | Part C, Step 2 using (R)-3- |
| | (5-(5-methyl-1H-pyrazol-1-yl)-1,3,4- | | (methoxymethyl)pyrrolidine as the |
| | thiadiazol-2-yl)-2-oxo-2H-pyran-6- | | “amine reagent” |
| | carboxamide | | |
| 2 | 3-methoxy-4-(4- | 461.15 | Prepared according to Example 1, |
| | (methoxymethyl)piperidin-1-yl)-N-(5- | | Part C, Step 2 using 4- |
| | (5-methyl-1H-pyrazol-1-yl)-1,3,4- | | (methoxymethyl)piperidine as the |
| | thiadiazol-2-yl)-2-oxo-2H-pyran-6- | | “amine reagent” |
| | carboxamide | | |
| 5 | 4-(((1S,2R)-2- | 433.10 | Prepared according to Example 1, |
| | hydroxycyclopentyl)amino)-3- | | Part C, Step 2 using (1R,2S)-2- |
| | methoxy-N-(5-(5-methyl-1H-pyrazol- | | aminocyclopentan-1-ol as the |
| | 1-yl)-1,3,4-thiadiazol-2-yl)-2-oxo- | | “amine reagent” |
| | 2H-pyran-6-carboxamide | | |
| Rac-6 | 4-(((1S,3S)-3- | 433.10 | Prepared according to Example 1, |
| | hydroxycyclopentyl)amino)-3- | | Part C, Step 2 using rac-(1,3-trans)- |
| | methoxy-N-(5-(5-methyl-1H-pyrazol- | | 3-aminocyclopentan-1-ol as the |
| | 1-yl)-1,3,4-thiadiazol-2-yl)-2-oxo- | | “amine reagent” |
| | 2H-pyran-6-carboxamide (Compound | | |
| | 6a*) | | |
| | 4-(((1R,3R)-3- | | |
| | hydroxycyclopentyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 6b*) | | |
| 8 | (R)-3-methoxy-N-(5-(5-methyl-1H- | 481.10 | Prepared according to Example 1. |
| | pyrazol-1-yl)-1,3,4-thiadiazol-2-yl)-4- | | Part C, Step 2 using (R)-3- |
| | (3-(methylsulfonyl)pyrrolidin-1-yl)-2- | | (methylsulfonyl)pyrrolidine as the |
| | oxo-2H-pyran-6-carboxamide | | “amine reagent” |
| 9 | 3-methoxy-4-(((1,3-cis)-3- | 433.10 | Prepared according to Example 1, |
| | methoxycyclobutyl)amino)-N-(5-(5- | | Part C, Step 2 using (1,3-cis)-3- |
| | methyl-1H-pyrazol-1-yl)-1,3,4- | | methoxycyclobutan-1-amine as the |
| | thiadiazol-2-yl)-2-oxo-2H-pyran-6- | | “amine reagent” |
| | carboxamide | | |
| 11 | 3-methoxy-4-(((1,4-trans)-4- | 461.10 | Prepared according to Example 1, |
| | methoxycyclohexyl)amino)-N-(5-(5- | | Part C, Step 2 using (1,4-trans)-4- |
| | methyl-1H-pyrazol-1-yl)-1,3,4- | | methoxycyclohexan-1-amine as the |
| | thiadiazol-2-yl)-2-oxo-2H-pyran-6- | | “amine reagent” |
| | carboxamide | | |
| 12 | 4-(4-(hydroxymethyl)piperidin-1-yl)- | 447.05 | Prepared according to Example 1, |
| | 3-methoxy-N-(5-(5-methyl-1H- | | Part C, Step 2 using piperidin-4- |
| | pyrazol-1-yl)-1,3,4-thiadiazol-2-yl)-2- | | ylmethanol as the “amine reagent” |
| | oxo-2H-pyran-6-carboxamide | | |
| 13 | 3-methoxy-4-(((3S,4R)-4- | 449.05 | Prepared according to Example 13 |
| | methoxytetrahydrofuran-3-yl)amino)- | | using (3S,4R)-4- |
| | N-(5-(5-methyl-1H-pyrazol-1-yl)- | | methoxytetrahydrofuran-3-amine as |
| | 1,3,4-thiadiazol-2-yl)-2-oxo-2H- | | the “amine reagent” |
| | pyran-6-carboxamide | | |
| Rac-14 | 3-methoxy-4-(((1S,2R)-2- | 447.10 | Prepared according to Example 1, |
| | methoxycyclopentyl)amino)-N-(5-(5- | | Part C, Step 2 using rac-(1,2-cis)-2- |
| | methyl-1H-pyrazol-1-yl)-1,3,4- | | methoxycyclopentan-1-amine as the |
| | thiadiazol-2-yl)-2-oxo-2H-pyran-6- | | “amine reagent” |
| | carboxamide (Compound 14a*) | | |
| | 3-methoxy-4-(((1R,2S)-2- | | |
| | methoxycyclopentyl)amino)-N-(5-(5- | | |
| | methyl-1H-pyrazol-1-yl)-1,3,4- | | |
| | thiadiazol-2-yl)-2-oxo-2H-pyran-6- | | |
| | carboxamide (Compound 14b*) | | |
| Rac-15 | 4-(((1S,2S)-2- | 433.10 | Prepared according to Example 1. |
| | hydroxycyclopentyl)amino)-3- | | Part C, Step 2 using rac-(1,2-trans)- |
| | methoxy-N-(5-(5-methyl-1H-pyrazol- | | 2-aminocyclopentan-1-ol as the |
| | 1-yl)-1,3,4-thiadiazol-2-yl)-2-oxo- | | “amine reagent” |
| | 2H-pyran-6-carboxamide (Compound | | |
| | 15a*) | | |
| | 4-(((1R,2R)-2- | | |
| | hydroxycyclopentyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 15b*) | | |
| 16 | 4-(((1,3-trans)-3- | 419.05 | Prepared according to Example 1, |
| | hydroxycyclobutyl)amino)-3- | | Part C, Step 2 using (1,3-trans)-3- |
| | methoxy-N-(5-(5-methyl-1H-pyrazol- | | aminocyclobutan-1-ol as the “amine |
| | 1-yl)-1,3,4-thiadiazol-2-yl)-2-oxo- | | reagent” |
| | 2H-pyran-6-carboxamide | | |
| 17 | 4-((2-(2,2- | 457.05 | Prepared according to Example 1, |
| | difluoroethoxy)ethyl)amino)-3- | | Part C, Step 2 using 2-(2,2- |
| | methoxy-N-(5-(5-methyl-1H-pyrazol- | | difluoroethoxy)ethan-1-amine as the |
| | 1-yl)-1,3,4-thiadiazol-2-yl)-2-oxo- | | “amine reagent” |
| | 2H-pyran-6-carboxamide | | |
| 18 | 4-(bicyclo[2.2.2]octan-1-ylamino)-3- | 457.10 | Prepared according to Example 1, |
| | methoxy-N-(5-(5-methyl-1H-pyrazol- | | Part C, Step 2 using |
| | 1-yl)-1,3,4-thiadiazol-2-yl)-2-oxo- | | bicyclo[2.2.2]octan-1-amine as the |
| | 2H-pyran-6-carboxamide | | “amine reagent” |
| 19 | 3-methoxy-4-(4-methoxy piperidin-1- | 447.05 | Prepared according to Example 1, |
| | yl)-N-(5-(5-methyl-1H-pyrazol-1-yl)- | | Part C, Step 2 using 4- |
| | 1,3,4-thiadiazol-2-yl)-2-oxo-2H- | | methoxypiperidine as the “amine |
| | pyran-6-carboxamide | | reagent” |
| 20 | (R)-3-methoxy-N-(5-(5-methyl-1H- | 433.10 | Prepared according to Example 1, |
| | pyrazol-1-yl)-1,3,4-thiadiazol-2-yl)-2- | | Part C, Step 2 using (R)- |
| | oxo-4-(((tetrahydrofuran-2- | | (tetrahydrofuran-2-yl)methanamine |
| | yl)methyl)amino)-2H-pyran-6- | | as the “amine reagent” |
| | carboxamide | | |
| 24 | (S)-4-((1-hydroxypropan-2- | 407.0 | Prepared according to Example 3 |
| | yl)amino)-3-methoxy-N-(5-(5- | | using (S)-2-aminopropan-1-ol as the |
| | methyl-1H-pyrazol-1-yl)-1,3,4- | | “amine reagent” |
| | thiadiazol-2-yl)-2-oxo-2H-pyran-6- | | |
| | carboxamide | | |
| 25 | (R)-4-((1-hydroxypropan-2- | 407.10 | Prepared according to Example 3 |
| | yl)amino)-3-methoxy-N-(5-(5- | | using (R)-2-aminopropan-1-ol as the |
| | methyl-1H-pyrazol-1-yl)-1,3,4- | | “amine reagent” |
| | thiadiazol-2-yl)-2-oxo-2H-pyran-6- | | |
| | carboxamide | | |
| 27 | 3-methoxy-4-(((1S,2R)-2- | 461.20 | Prepared according to Example 7 |
| | methoxycyclohexyl)amino)-N-(5-(5- | | using (1S,2R)-2- |
| | methyl-1H-pyrazol-1-yl)-1,3,4- | | methoxycyclohexan-1-amine as the |
| | thiadiazol-2-yl)-2-oxo-2H-pyran-6- | | “amine reagent” |
| | carboxamide | | |
| 30 | (R)-4-(3-hydroxypyrrolidin-1-yl)-3- | 419.1 | Prepared according to Example 1, |
| | methoxy-N-(5-(5-methyl-1H-pyrazol- | | Part C, Step 2 using (R)-pyrrolidin-3- |
| | 1-yl)-1,3,4-thiadiazol-2-yl)-2-oxo- | | ol as the “amine reagent” |
| | 2H-pyran-6-carboxamide | | |
| 31 | (R)-5-methoxy-4-(3- | 433.1 | Prepared according to Example 1, |
| | methoxypyrrolidin-1-yl)-N-(5-(5- | | Part C, Step 2 using (R)-3- |
| | methyl-1H-pyrazol-1-yl)-1,3,4- | | methoxypyrrolidine as the “amine |
| | thiadiazol-2-yl)-6-oxo-6H-pyran-2- | | reagent” |
| | carboxamide | | |
| 32 | 5-methoxy-4-(3- | 421.0 | Prepared according to Example 1, |
| | methoxypropylamino)-N-(5-(5- | | Part C, Step 2 using 3- |
| | methyl-1H-pyrazol-1-yl)-1,3,4- | | methoxypropan-1-amine as the |
| | thiadiazol-2-yl)-6-oxo-6H-pyran-2- | | “amine reagent” |
| | carboxamide | | |
| 33 | 4-(2-hydroxy-2-methylpropylamino)- | 421.0 | Prepared according to Example 1, |
| | 5-methoxy-N-(5-(5-methyl-1H- | | Part C, Step 2 using 1-amino-2- |
| | pyrazol-1-yl)-1,3,4-thiadiazol-2-yl)-6- | | methylpropan-2-ol as the “amine |
| | oxo-6H-pyran-2-carboxamide | | reagent” |
| 34 | (R)-4-(2-(hydroxymethyl)pyrrolidin- | 433.0 | Prepared according to Example 1, |
| | 1-yl)-5-methoxy-N-(5-(5-methyl-1H- | | Part C, Step 2 using (R)-pyrrolidin-2- |
| | pyrazol-1-yl)-1,3,4-thiadiazol-2-yl)-6- | | ylmethanol as the “amine reagent” |
| | oxo-6H-pyran-2-carboxamide | | |
| 35 | 4-((3-hydroxypropyl)amino)-3- | 407.0 | Prepared according to Example 1, |
| | methoxy-N-(5-(5-methyl-1H-pyrazol- | | Part C, Step 2 using 3-aminopropan- |
| | 1-yl)-1,3,4-thiadiazol-2-yl)-2-oxo- | | 1-ol as the “amine reagent” |
| | 2H-pyran-6-carboxamide | | |
| Rac-36 | 3-methoxy-4-(((1R,2R)-2- | 433.0 | Prepared according to Example 1, |
| | methoxycyclobutyl)amino)-N-(5-(5- | | Part C, Step 2 using rac-(1,2-trans)- |
| | methyl-1H-pyrazol-1-yl)-1,3,4- | | 2-methoxycyclobutan-1-amine as the |
| | thiadiazol-2-yl)-2-oxo-2H-pyran-6- | | “amine reagent” |
| | carboxamide (Compound 36a*) | | |
| | 3-methoxy-4-(((1S,2S)-2- | | |
| | methoxycyclobutyl)amino)-N-(5-(5- | | |
| | methyl-1H-pyrazol-1-yl)-1,3,4- | | |
| | thiadiazol-2-yl)-2-oxo-2H-pyran-6- | | |
| | carboxamide (Compound 36b*) | | |
| Rac-37 | 4-(((1R,2R)-2- | 419.0 | Prepared according to Example 1, |
| | hydroxycyclobutyl)amino)-3- | | Part C, Step 2 using rac-(1,2-cis)-2- |
| | methoxy-N-(5-(5-methyl-1H-pyrazol- | | aminocyclobutan-1-ol as the “amine |
| | 1-yl)-1,3,4-thiadiazol-2-yl)-2-oxo- | | reagent” |
| | 2H-pyran-6-carboxamide (Compound | | |
| | 37a*) | | |
| | 4-(((1S,2S)-2- | | |
| | hydroxycyclobutyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 37b*) | | |
| 38 | (R)-3-methoxy-4-(2- | 447.1 | Prepared according to Example 1, |
| | (methoxymethyl)pyrrolidin-1-yl)-N- | | Part C, Step 2 using (R)-2- |
| | (5-(5-methyl-1H-pyrazol-1-yl)-1,3,4- | | (methoxymethyl)pyrrolidine as the |
| | thiadiazol-2-yl)-2-oxo-2H-pyran-6- | | “amine reagent” |
| | carboxamide | | |
| 39 | (S)-3-methoxy-4-(2- | 447.1 | Prepared according to Example 1, |
| | (methoxymethyl)pyrrolidin-1-yl)-N- | | Part C, Step 2 using (S)-2- |
| | (5-(5-methyl-1H-pyrazol-1-yl)-1,3,4- | | (methoxymethyl)pyrrolidine as the |
| | thiadiazol-2-yl)-2-oxo-2H-pyran-6- | | “amine reagent” |
| | carboxamide | | |
| 40 | (S)-4-(2-(hydroxymethyl)pyrrolidin- | 433.1 | Prepared according to Example 1, |
| | 1-yl)-3-methoxy-N-(5-(5-methyl-1H- | | Part C, Step 2 using (S)-pyrrolidin-2- |
| | pyrazol-1-yl)-1,3,4-thiadiazol-2-yl)-2- | | ylmethanol as the “amine reagent” |
| | oxo-2H-pyran-6-carboxamide | | |
| 41 | 4-{bicyclo[1.1.1]pentan-1-ylamino}- | 448.0 | Prepared according to Example 10, |
| | N-[5-(3-chloro-1-methylpyrrol-2-yl)- | | Step 7 using bicyclo[1.1.1]pentan-1- |
| | 1,3,4-thiadiazol-2-yl]-5-methoxy-6- | | amine as the “amine reagent” |
| | oxopyran-2-carboxamide | | |
| 42 | 3-methoxy-N-(5-(5-methyl-1H- | 417.0 | Prepared according to Example 1, |
| | pyrazol-1-yl)-1,3,4-thiadiazol-2-yl)-2- | | Part C, Step 2 using pyrrolidin-2-one |
| | oxo-4-(2-oxopyrrolidin-1-yl)-2H- | | as the “amine reagent” |
| | pyran-6-carboxamide | | |
| Rac-43 | 4-(((1R,2S)-2- | 419.0 | Prepared according to Example 1, |
| | hydroxycyclobutyl)amino)-3- | | Part C, Step 2 using rac-(1,2-cis)-2- |
| | methoxy-N-(5-(5-methyl-1H-pyrazol- | | aminocyclobutan-1-ol as the “amine |
| | 1-yl)-1,3,4-thiadiazol-2-yl)-2-oxo- | | reagent” |
| | 2H-pyran-6-carboxamide (Compound | | |
| | 43a*) | | |
| | 4-(((1S,2R)-2- | | |
| | hydroxycyclobutyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 43b*) | | |
| 44 | 3-methoxy-4-((3- | 459.1 | Prepared according to Example 1, |
| | (methoxymethyl)bicyclo[1.1.1]pentan- | | Part C, Step 2 using 3- |
| | 1-yl)amino)-N-(5-(5-methyl-1H- | | methoxybicyclo[1.1.1]pentan-1- |
| | pyrazol-1-yl)-1,3,4-thiadiazol-2-yl)-2- | | amine as the “amine reagent” |
| | oxo-2H-pyran-6-carboxamide | | |
| 45 | 4-((3-cyanobicyclo[1.1.1]pentan-1- | 440.0 | Prepared according to Example 1, |
| | yl)amino)-3-methoxy-N-(5-(5- | | Part C, Step 2 using 3- |
| | methyl-1H-pyrazol-1-yl)-1,3,4- | | aminobicyclo[1.1.1]pentane-1- |
| | thiadiazol-2-yl)-2-oxo-2H-pyran-6- | | carbonitrile as the “amine reagent” |
| | carboxamide | | |
| 46 | 4-(4-hydroxypiperidin-1-yl)-3- | 433.1 | Prepared according to Example 1, |
| | methoxy-N-(5-(5-methyl-1H-pyrazol- | | Part C, Step 2 using piperidin-4-ol as |
| | 1-yl)-1,3,4-thiadiazol-2-yl)-2-oxo- | | the “amine reagent” |
| | 2H-pyran-6-carboxamide | | |
| 47* | N-(5-(3-chloro-1-methyl-1H-pyrrol-2- | 480.0 | Compounds 47* and 48* were |
| | yl)-1,3,4-thiadiazol-2-yl)-3-methoxy- | | prepared and stereoisomers separated |
| | 4-(((1S,2R)-2- | | according to Example 2 using 4- |
| | methoxycyclopentyl)amino)-2-oxo- | | bromo-N-(5-(3-chloro-1-methyl-1H- |
| | 2H-pyran-6-carboxamide | | pyrrol-2-yl)-1,3,4-thiadiazol-2-yl)-3- |
| 48* | N-(5-(3-chloro-1-methyl-1H-pyrrol-2- | 480.0 | methoxy-2-oxo-2H-pyran-6- |
| | yl)-1,3,4-thiadiazol-2-yl)-3-methoxy- | | carboxamide (product of Example |
| | 4-(((1R,2S)-2- | | 10, Step 6) as the “halo-pyrone |
| | methoxycyclopentyl)amino)-2-oxo- | | reagent” and rac-(1,2-cis)-2- |
| | 2H-pyran-6-carboxamide | | methoxycyclopentan-1-amine as the |
| | | | “amine reagent”. |
| | | | Compound 47* was the first eluting |
| | | | peak; Compound 48* was the second |
| | | | eluting peak. |
| 49 | (S)-3-methoxy-4-((2-methoxy-1- | 483.1 | Prepared according to Example 9 |
| | phenylethyl)amino)-N-(5-(5-methyl- | | using (S)-2-methoxy-1-phenylethan- |
| | 1H-pyrazol-1-yl)-1,3,4-thiadiazol-2- | | 1-amine as the “amine reagent” |
| | yl)-2-oxo-2H-pyran-6-carboxamide | | |
| 50 | 4-((2-oxaspiro[3.3]heptan-6- | 444.1 | Prepared according to Example 1, |
| | yl)amino)-3-methoxy-N-(5-(5- | | Part C, Step 2 using 2- |
| | methyl-1H-pyrazol-1-yl)-1,3,4- | | oxaspiro[3.3]heptan-6-amine as the |
| | thiadiazol-2-yl)-2-oxo-2H-pyran-6- | | “amine reagent” |
| | carboxamide | | |
| 51 | 4-((2-oxabicyclo[2.1.1]hexan-4- | 431.0 | Prepared according to Example 1, |
| | yl)amino)-3-methoxy-N-(5-(5- | | Part C, Step 2 using 2- |
| | methyl-1H-pyrazol-1-yl)-1,3,4- | | oxabicyclo[2.1.1]hexan-4-amine as |
| | thiadiazol-2-yl)-2-oxo-2H-pyran-6- | | the “amine reagent” |
| | carboxamide | | |
| 52 | 3-methoxy-4-(((1R,2S)-2- | 461.1 | Prepared according to Example 13 |
| | methoxycyclohexyl)amino)-N-(5-(5- | | using (1R,2S)-2-methoxycyclohexan- |
| | methyl-1H-pyrazol-1-yl)-1,3,4- | | 1-amine as the “amine reagent” |
| | thiadiazol-2-yl)-2-oxo-2H-pyran-6- | | |
| | carboxamide | | |
| Rac-55 | 4-(((1S,2R)-2- | 433.15 | Prepared according to Example 1, |
| | (hydroxymethyl)cyclopentyl)amino)- | | Part C, Step 2 using rac-((1,2-cis)-2- |
| | 3-methoxy-N-(5-(5-methyl-1H- | | aminocyclopentyl)methanol as the |
| | pyrazol-1-yl)-1,3,4-thiadiazol-2-yl)-2- | | “amine reagent” |
| | oxo-2H-pyran-6-carboxamide | | |
| | (Compound 55a*) | | |
| | 4-(((1R,2S)-2- | | |
| | (hydroxymethyl)cyclopentyl)amino)- | | |
| | 3-methoxy-N-(5-(5-methyl-1H- | | |
| | pyrazol-1-yl)-1,3,4-thiadiazol-2-yl)-2- | | |
| | oxo-2H-pyran-6-carboxamide | | |
| | (Compound 55b*) | | |
| 57 | (S)-3-methoxy-4-(3- | 433.15 | Prepared according to Example 1, |
| | methoxypyrrolidin-1-yl)-N-(5-(5- | | Part C, Step 2 using (S)-3- |
| | methyl-1H-pyrazol-1-yl)-1,3,4- | | methoxypyrrolidine as the “amine |
| | thiadiazol-2-yl)-2-oxo-2H-pyran-6- | | reagent” |
| | carboxamide | | |
| 58 | (S)-4-(3-(hydroxymethyl)pyrrolidin- | 433.05 | Prepared according to Example 1, |
| | 1-yl)-3-methoxy-N-(5-(5-methyl-1H- | | Part C, Step 2 using (S)-pyrrolidin-3- |
| | pyrazol-1-yl)-1,3,4-thiadiazol-2-yl)-2- | | ylmethanol |
| | oxo-2H-pyran-6-carboxamide | | |
| 59 | 4-((3S,4R)-3,4-dimethoxypyrrolidin- | 463.05 | Prepared according to Example 1, |
| | 1-yl)-3-methoxy-N-R(5-(5-methyl- | | Part C, Step 2 using (3S,4R)-3,4- |
| | 1H-pyrazol-1-yl)-1,3,4-thiadiazol-2- | | dimethoxypyrrolidine as the “amine |
| | yl)-2-oxo-2H-pyran-6-carboxamide | | reagent” |
| 60 | (S)-3-methoxy-N-(5-(5-methyl-1H- | 481.10 | Prepared according to Example 1, |
| | pyrazol-1-yl)-1,3,4-thiadiazol-2-yl)-4- | | Part C, Step 2 using (S)-3- |
| | (3-(methylsulfonyl)pyrrolidin-1-yl)-2- | | (methylsulfonyl)pyrrolidine as the |
| | oxo-2H-pyran-6-carboxamide | | “amine reagent” |
| Rac-61 | 4-(((1S,3R)-3- | 432.12 | Prepared according to Example 1, |
| | hydroxycyclopentyl)amino)-3- | | Part C, Step 2 using rac-(1,3-cis)-3- |
| | methoxy-N-(5-(5-methyl-1H-pyrazol- | | aminocyclopentan-1-ol as the |
| | 1-yl)-1,3,4-thiadiazol-2-yl)-2-oxo- | | “amine reagent” |
| | 2H-pyran-6-carboxamide (Compound | | |
| | 61a*) | | |
| | 4-(((1R,3S)-3- | | |
| | hydroxycyclopentyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 61b*) | | |
| 62 | (S)-4-((1,1- | 467.05 | Prepared according to Example 1, |
| | dioxidotetrahydrothiophen-3- | | Part C, Step 2 using (S)-3- |
| | yl)amino)-3-methoxy-N-(5-(5- | | aminotetrahydrothiophene 1,1- |
| | methyl-1H-pyrazol-1-yl)-1,3,4- | | dioxide as the “amine reagent” |
| | thiadiazol-2-yl)-2-oxo-2H-pyran-6- | | |
| | carboxamide | | |
| 63 | 4-(bis(2-methoxyethyl)amino)-3- | 465.10 | Prepared according to Example 1, |
| | methoxy-N-(5-(5-methyl-1H-pyrazol- | | Part C, Step 2 using bis(2- |
| | 1-yl)-1,3,4-thiadiazol-2-yl)-2-oxo- | | methoxyethyl)amine as the “amine |
| | 2H-pyran-6-carboxamide | | reagent” |
| 64 | (S)-3-methoxy-4-((1-methoxypropan- | 421.20 | Prepared according to Example 1, |
| | 2-yl)amino)-N-(5-(5-methyl-1H- | | Part C, Step 2 using (S)-1- |
| | pyrazol-1-yl)-1,3,4-thiadiazol-2-yl)-2- | | methoxypropan-2-amine as the |
| | oxo-2H-pyran-6-carboxamide | | “amine reagent” |
| 65 | 4-(((1,3-cis)-3- | 419.20 | Prepared according to Example 1, |
| | hydroxycyclobutyl)amino)-3- | | Part C, Step 2 using (1,3-cis)-3- |
| | methoxy-N-(5-(5-methyl-1H-pyrazol- | | aminocyclobutan-1-o as the “amine |
| | 1-yl)-1,3,4-thiadiazol-2-yl)-2-oxo- | | reagent” |
| | 2H-pyran-6-carboxamide | | |
| 66 | (S)-3-methoxy-N-(5-(5-methyl-1H- | 433.05 | Prepared according to Example 1, |
| | pyrazol-1-yl)-1,3,4-thiadiazol-2-yl)-2- | | Part C, Step 2 using (S)- |
| | oxo-4-(((tetrahydrofuran-2- | | (tetrahydrofuran-2-yl)methanamine |
| | yl)methyl)amino)-2H-pyran-6- | | as the “amine reagent” |
| | carboxamide | | |
| Rac-67 | 3-methoxy-4-(((1R,2S)-2- | 443.10 | Prepared according to Example 1, |
| | methoxycyclobutyl)amino)-N-(5-(5- | | Part C, Step 2 using rac-(1,2-cis)-2- |
| | methyl-1H-pyrazol-1-yl)-1,3,4- | | methoxycyclobutan-1-amine as the |
| | thiadiazol-2-yl)-2-oxo-2H-pyran-6- | | “amine reagent” |
| | carboxamide (Compound 67a*) | | |
| | 3-methoxy-4-(((1S,2R)-2- | | |
| | methoxycyclobutyl)amino)-N-(5-(5- | | |
| | methyl-1H-pyrazol-1-yl)-1,3,4- | | |
| | thiadiazol-2-yl)-2-oxo-2H-pyran-6- | | |
| | carboxamide (Compound 67b*) | | |
| Rac-68 | 4-((3S,4R)-3-hydroxy-4- | 449.10 | Prepared according to Example 1. |
| | methoxypyrrolidin-1-yl)-3-methoxy- | | Part C, Step 2 using rac-(3,4-cis)-4- |
| | N-(5-(5-methyl-1H-pyrazol-1-yl)- | | methoxypyrrolidin-3-ol as the |
| | 1,3,4-thiadiazol-2-yl)-2-oxo-2H- | | “amine reagent” |
| | pyran-6-carboxamide (Compound | | |
| | 68a*) | | |
| | 4-((3R,4S)-3-hydroxy-4- | | |
| | methoxypyrrolidin-1-yl)-3-methoxy- | | |
| | N-(5-(5-methyl-1H-pyrazol-1-yl)- | | |
| | 1,3,4-thiadiazol-2-yl)-2-oxo-2H- | | |
| | pyran-6-carboxamide (Compound | | |
| | 68b*) | | |
| 69 | 3-methoxy-4-(((1,3-trans)-3- | 433.12 | Prepared according to Example 1, |
| | methoxycyclobutyl)amino)-N-(5-(5- | | Part C, Step 2 using (1,3-trans)-3- |
| | methyl-1H-pyrazol-1-yl)-1,3,4- | | methoxycyclobutan-1-amine as the |
| | thiadiazol-2-yl)-2-oxo-2H-pyran-6- | | “amine reagent” |
| | carboxamide | | |
| 70 | 3-methoxy-4-(((1S,2S)-2- | 461.10 | Prepared according to Example 13 |
| | methoxycyclohexyl)amino)-N-(5-(5- | | using (1S,2S)-2-methoxycyclohexan- |
| | methyl-1H-pyrazol-1-yl)-1,3,4- | | 1-amine as the “amine reagent” |
| | thiadiazol-2-yl)-2-oxo-2H-pyran-6- | | |
| | carboxamide | | |
| 71* | (S)-3-methoxy-N-(5-(5-methyl-1H- | 446.12 | Compounds 71* and 72* were |
| | pyrazol-1-yl)-1,3,4-thiadiazol-2-yl)-4- | | prepared and stereoisomers separated |
| | ((1-methyl-2-oxopyrrolidin-3- | | according to Example 2 using 3- |
| | yl)amino)-2-oxo-2H-pyran-6- | | amino-1-methylpyrrolidin-2-one as |
| | carboxamide | | the “amine reagent”. |
| 72* | (R)-3-methoxy-N-(5-(5-methyl-1H- | 446.12 | Compound 71* was the first eluting |
| | pyrazol-1-yl)-1,3,4-thiadiazol-2-yl)-4- | | peak; Compound 72* was the second |
| | ((1-methyl-2-oxopyrrolidin-3- | | eluting peak. |
| | yl)amino)-2-oxo-2H-pyran-6- | | |
| | carboxamide | | |
| 76 | 3-methoxy-4-((1-methoxy-2- | 435.14 | Prepared according to Example 13 |
| | methylpropan-2-yl)amino)-N-(5-(5- | | using 1-methoxy-2-methylpropan-2- |
| | methyl-1H-pyrazol-1-yl)-1,3,4- | | amine as the “amine reagent” |
| | thiadiazol-2-yl)-2-oxo-2H-pyran-6- | | |
| | carboxamide | | |
| 77 | (S)-3-methoxy-4-((1-methoxy-3- | 449.15 | Prepared according to Example 13 |
| | methylbutan-2-yl)amino)-N-(5-(5- | | using (S)-1-methoxy-3-methylbutan- |
| | methyl-1H-pyrazol-1-yl)-1,3,4- | | 2-amine as the “amine reagent” |
| | thiadiazol-2-yl)-2-oxo-2H-pyran-6- | | |
| | carboxamide | | |
| 78 | (R)-3-methoxy-4-((1-methoxy-3- | 449.15 | Prepared according to Example 13 |
| | methylbutan-2-yl)amino)-N-(5-(5- | | using (R)-1-methoxy-3-methylbutan- |
| | methyl-1H-pyrazol-1-yl)-1,3,4- | | 2-amine as the “amine reagent” |
| | thiadiazol-2-yl)-2-oxo-2H-pyran-6- | | |
| | carboxamide | | |
| Rac-81 | 4-(((1S,2R)-1-hydroxy-2,3-dihydro- | 481.05 | Prepared according to Example 13 |
| | 1H-inden-2-yl)amino)-3-methoxy-N- | | using rac-(1,2-cis)-2-amino-2,3- |
| | (5-(5-methyl-1H-pyrazol-1-yl)-1,3,4- | | dihydro-1H-inden-1-ol as the “amine |
| | thiadiazol-2-yl)-2-oxo-2H-pyran-6- | | reagent” |
| | carboxamide (Compound 81a*) | | |
| | 4-(((1R,2S)-1-hydroxy-2,3-dihydro- | | |
| | 1H-inden-2-yl)amino)-3-methoxy-N- | | |
| | (5-(5-methyl-1H-pyrazol-1-yl)-1,3,4- | | |
| | thiadiazol-2-yl)-2-oxo-2H-pyran-6- | | |
| | carboxamide (Compound 81b*) | | |
| 82 | (R)-4-((1,1- | 467.10 | Prepared according to Example 1, |
| | dioxidotetrahydrothiophen-3- | | Part C, Step 2 using (R)-3- |
| | yl)amino)-3-methoxy-N-(5-(5- | | aminotetrahydrothiophene 1,1- |
| | methyl-1H-pyrazol-1-yl)-1,3,4- | | dioxide as the “amine reagent” |
| | thiadiazol-2-yl)-2-oxo-2H-pyran-6- | | |
| | carboxamide | | |
| 83 | N-(5-(3-chloro-1-methyl-1H-pyrrol-2- | 466.10 | Prepared according to Example 10, |
| | yl)-1,3,4-thiadiazol-2-yl)-4-(((1S,2R)- | | Step 7 using (1R,2S)-2- |
| | 2-hydroxycyclopentyl)amino)-3- | | aminocyclopentan-1-ol as the |
| | methoxy-2-oxo-2H-pyran-6- | | “amine reagent” |
| | carboxamide | | |
| 84 | N-(5-(3-chloro-1-methyl-1H-pyrrol-2- | 484.10 | Prepared according to Example 10, |
| | yl)-1,3,4-thiadiazol-2-yl)-4-((1,3- | | Step 7 using 1,3-dimethoxypropan-2- |
| | dimethoxypropan-2-yl)amino)-3- | | amine as the “amine reagent” |
| | methoxy-2-oxo-2H-pyran-6- | | |
| | carboxamide | | |
| 85 | N-(5-(3-chloro-1-methyl-1H-pyrrol-2- | 510.10 | Prepared according to Example 16 |
| | yl)-1,3,4-thiadiazol-2-yl)-4-(((1S,2R)- | | using (1R,2S)-2-aminocyclopentan- |
| | 2-hydroxycyclopentyl)amino)-3-(2- | | 1-ol as the “amine reagent” in Step |
| | methoxyethoxy)-2-oxo-2H-pyran-6- | | 2 |
| | carboxamide | | |
| 86 | N-(5-(3-chloro-1-methyl-1H-pyrrol-2- | 449.15 | Prepared according to Example 16 |
| | yl)-1,3,4-thiadiazol-2-yl)-4-(((1R,2S)- | | using (1S,2R)-2-aminocyclopentan- |
| | 2-hydroxycyclopentyl)amino)-3-(2- | | 1-ol as the “amine reagent” in Step |
| | methoxyethoxy)-2-oxo-2H-pyran-6- | | 2 |
| | carboxamide | | |
| 88 | (R)-4-(3-(hydroxymethyl)pyrrolidin- | 433.0 | Prepared according to Example 1, |
| | 1-yl)-3-methoxy-N-(5-(5-methyl-1H- | | Part C, Step 2 using (R)-pyrrolidin-3- |
| | pyrazol-1-yl)-1,3,4-thiadiazol-2-yl)-2- | | ylmethanol as the “amine reagent” |
| | oxo-2H-pyran-6-carboxamide | | |
| 89 | 4-((2,2-difluoroethyl)amino)-3- | 413.00 | Prepared according to Example 1, |
| | methoxy-N-(5-(5-methyl-1H-pyrazol- | | Part C, Step 2 using 2,2- |
| | 1-yl)-1,3,4-thiadiazol-2-yl)-2-oxo- | | difluoroethan-1-amine as the “amine |
| | 2H-pyran-6-carboxamide | | reagent” |
| 90 | 3-methoxy-N-(5-(5-methyl-1H- | 431.00 | Prepared according to Example 1, |
| | pyrazol-1-yl)-1,3,4-thiadiazol-2-yl)-2- | | Part C, Step 2 using 2,2,2- |
| | oxo-4-((2,2,2-trifluoroethyl)amino)- | | trifluoroethan-1-amine as the “amine |
| | 2H-pyran-6-carboxamide | | reagent” |
| Rac-91 | 4-((3S,4S)-3-hydroxy-4- | 449.05 | Prepared according to Example 1, |
| | methoxypyrrolidin-1-yl)-3-methoxy- | | Part C, Step 2 using rac-(3,4-trans)- |
| | N-(5-(5-methyl-1H-pyrazol-1-yl)- | | 4-methoxypyrrolidin-3-ol as the |
| | 1,3,4-thiadiazol-2-yl)-2-oxo-2H- | | “amine reagent” |
| | pyran-6-carboxamide (Compound | | |
| | 91a*) | | |
| | 4-((3R,4R)-3-hydroxy-4- | | |
| | methoxypyrrolidin-1-yl)-3-methoxy- | | |
| | N-(5-(5-methyl-1H-pyrazol-1-yl)- | | |
| | 1,3,4-thiadiazol-2-yl)-2-oxo-2H- | | |
| | pyran-6-carboxamide (Compound | | |
| | 91b*) | | |
| Rac-92 | 4-((3S,4S)-3,4-dimethoxypyrrolidin- | 463.05 | Prepared according to Example 1, |
| | 1-yl)-3-methoxy-N-(5-(5-methyl-1H- | | Part C, Step 2 using rac-(3,4-trans)- |
| | pyrazol-1-yl)-1,3,4-thiadiazol-2-yl)-2- | | 3,4-dimethoxypyrrolidine as the |
| | oxo-2H-pyran-6-carboxamide | | “amine reagent” |
| | (Compound 92a*) | | |
| | 4-((3S,4S)-3,4-dimethoxypyrrolidin- | | |
| | 1-yl)-3-methoxy-N-(5-(5-methyl-1H- | | |
| | pyrazol-1-yl)-1,3,4-thiadiazol-2-yl)-2- | | |
| | oxo-2H-pyran-6-carboxamide | | |
| | (Compound 92b*) | | |
| 93 | (S)-3-methoxy-N-(5-(5-methyl-1H- | 419.5 | Prepared according to Example 1, |
| | pyrazol-1-yl)-1,3,4-thiadiazol-2-yl)-4- | | Part C, Step 2 using (S)-oxetan-2- |
| | ((oxetan-2-ylmethyl)amino)-2-oxo- | | ylmethanamine as the “amine |
| | 2H-pyran-6-carboxamide | | reagent” |
| 95 | (S)-3-methoxy-4-(2- | 433.15 | Prepared according to Example 13 |
| | (methoxymethyl)azetidin-1-yl)-N-(5- | | using (S)-2- |
| | (5-methyl-1H-pyrazol-1-yl)-1,3,4- | | (methoxymethyl)azetidine as the |
| | thiadiazol-2-yl)-2-oxo-2H-pyran-6- | | “amine reagent” |
| | carboxamide | | |
| 96 | 3-methoxy-N-(5-(5-methyl-1H- | 431.05 | Prepared according to Example 13 |
| | pyrazol-1-yl)-1,3,4-thiadiazol-2-yl)-2- | | using piperidin-2-one as the “amine |
| | oxo-4-(2-oxopiperidin-1-y])-2H- | | reagent” |
| | pyran-6-carboxamide | | |
| 97 | 3-methoxy-4-((1- | 433.10 | Prepared according to Example 13 |
| | (methoxymethyl)cyclopropyl)amino)- | | using 1- |
| | N-(5-(5-methyl-1H-pyrazol-1-yl)- | | (methoxymethyl)cyclopropan-1- |
| | 1,3,4-thiadiazol-2-yl)-2-oxo-2H- | | amine as the “amine reagent” |
| | pyran-6-carboxamide | | |
| 98 | 3-methoxy-4-((3- | 449.10 | Prepared according to Example 13 |
| | (methoxymethyl)oxetan-3-yl)amino)- | | using 3-(methoxymethyl)oxetan-3- |
| | N-(5-(5-methyl-1H-pyrazol-1-yl)- | | amine as the “amine reagent” |
| | 1,3,4-thiadiazol-2-yl)-2-oxo-2H- | | |
| | pyran-6-carboxamide | | |
| 99 | 4-(1,1-dioxidoisothiazolidin-2-yl)-3- | 453.0 | Prepared according to Example 13 |
| | methoxy-N-(5-(5-methyl-1H-pyrazol- | | using isothiazolidine 1,1-dioxide as |
| | 1-yl)-1,3,4-thiadiazol-2-yl)-2-oxo- | | the “amine reagent” |
| | 2H-pyran-6-carboxamide | | |
| 102 | 3-methoxy-4-(((3R,4S)-4- | 449.1 | Prepared according to Example 13 |
| | methoxytetrahydrofuran-3-yl)amino)- | | using (3R,4S)-4- |
| | N-(5-(5-methyl-1H-pyrazol-1-yl)- | | methoxytetrahydrofuran-3-amine as |
| | 1,3,4-thiadiazol-2-yl)-2-oxo-2H- | | the “amine reagent” |
| | pyran-6-carboxamide | | |
| 103 | 4-(benzylamino)-3-methoxy-N-(5-(5- | 439.05 | Prepared according to Example 1, |
| | methyl-1H-pyrazol-1-yl)-1,3,4- | | Part C, Step 2 using |
| | thiadiazol-2-yl)-2-oxo-2H-pyran-6- | | phenylmethanamine as the “amine |
| | carboxamide | | reagent” |
| 104 | 3-methoxy-N-(5-(5-methyl-1H- | 405.05 | Prepared according to Example 3 |
| | pyrazol-1-yl)-1,3,4-thiadiazol-2-yl)-4- | | using oxetan-3-amine as the “amine |
| | (oxetan-3-ylamino)-2-oxo-2H-pyran- | | reagent” |
| | 6-carboxamide | | |
| 105 | 3-methoxy-N-(5-(5-methyl-1H- | 363.10 | Prepared according to Example 1, |
| | pyrazol-1-yl)-1,3,4-thiadiazol-2-yl)-4- | | Part C, Step 2 using methylamine |
| | (methylamino)-2-oxo-2H-pyran-6- | | hydrochloride as the “amine |
| | carboxamide | | reagent” |
| 106 | 4-(dimethylamino)-3-methoxy-N-(5- | 377.0 | Prepared according to Example 3 |
| | (5-methyl-1H-pyrazol-1-yl)-1,3,4- | | using dimethylamine hydrochloride |
| | thiadiazol-2-yl)-2-oxo-2H-pyran-6- | | as the “amine reagent” |
| | carboxamide | | |
| Rac-107 | 3-methoxy-4-(((1S,2S)-2- | 447.05 | Prepared according to Example 1, |
| | methoxycyclopentyl)amino)-N-(5-(5- | | Part C, Step 2 using rac-(1,2-trans)- |
| | methyl-1H-pyrazol-1-yl)-1,3,4- | | 2-methoxycyclopentan-1-amine as |
| | thiadiazol-2-yl)-2-oxo-2H-pyran-6- | | the “amine reagent” |
| | carboxamide (Compound 107a*) | | |
| | 3-methoxy-4-(((1R,2R)-2- | | |
| | methoxycyclopentyl)amino)-N-(5-(5- | | |
| | methyl-1H-pyrazol-1-yl)-1,3,4- | | |
| | thiadiazol-2-yl)-2-oxo-2H-pyran-6- | | |
| | carboxamide (Compound 107b*) | | |
| 110 | (S)-4-(3-hydroxypyrrolidin-1-yl)-3- | 419.05 | Prepared according to Example 1, |
| | methoxy-N-(5-(5-methyl-1H-pyrazol- | | Part C, Step 2 using (S)-pyrrolidin-3- |
| | 1-yl)-1,3,4-thiadiazol-2-yl)-2-oxo- | | ol as the “amine reagent” |
| | 2H-pyran-6-carboxamide | | |
| 111 | 3-methoxy-4-((3- | 445.10 | Prepared according to Example 1, |
| | methoxybicyclo[1.1.1]pentan-1- | | Part C, Step 2 using 3- |
| | yl)amino)-N-(5-(5-methyl-1H- | | methoxybicyclo[1.1.1]pentan-1- |
| | pyrazol-1-yl)-1,3,4-thiadiazol-2-yl)-2- | | amine as the “amine reagent” |
| | oxo-2H-pyran-6-carboxamide | | |
| 112 | 4-(((1R,2S)-2- | 433.10 | Prepared according to Example 1, |
| | hydroxycyclopentyl)amino)-3- | | Part C, Step 2 using (1S,2R)-2- |
| | methoxy-N-(5-(5-methyl-1H-pyrazol- | | aminocyclopentan-1-ol as the |
| | 1-yl)-1,3,4-thiadiazol-2-yl)-2-oxo- | | “amine reagent” |
| | 2H-pyran-6-carboxamide | | |
| 113 | 4-(((3S,4S)-4- | 435.00 | Prepared according to Example 1, |
| | hydroxytetrahydrofuran-3-yl)amino)- | | Part C, Step 2 using (3S,4S)-4- |
| | 3-methoxy-N-(5-(5-methyl-1H- | | aminotetrahydrofuran-3-ol as the |
| | pyrazol-1-yl)-1,3,4-thiadiazol-2-yl)-2- | | “amine reagent” |
| | oxo-2H-pyran-6-carboxamide | | |
| 114 | (R)-3-methoxy-4-((2-methoxy-1- | 483.1 | Prepared according to Example 1, |
| | phenylethyl)amino)-N-(5-(5-methyl- | | Part C, Step 2 using (R)-2-methoxy- |
| | 1H-pyrazol-1-yl)-1,3,4-thiadiazol-2- | | 1-phenylethan-1-amine as the |
| | yl)-2-oxo-2H-pyran-6-carboxamide | | “amine reagent” |
| 115 | 4-((3- | 464.95 | Prepared according to Example 1, |
| | (difluoromethyl)bicyclo[1.1.1]pentan- | | Part C, Step 2 using 3- |
| | 1-yl)amino)-3-methoxy-N-(5-(5- | | (difluoromethyl)bicyclo[1.1.1]pentan- |
| | methyl-1H-pyrazol-1-yl)-1,3,4- | | 1-amine as the “amine reagent” |
| | thiadiazol-2-yl)-2-oxo-2H-pyran-6- | | |
| | carboxamide | | |
| 116 | 4-((3- | 454.1 | Prepared according to Example 1, |
| | (cyanomethyl)bicyclo[1.1.1]pentan-1- | | Part C, Step 2 using 2-(3- |
| | yl)amino)-3-methoxy-N-(5-(5- | | aminobicyclo[1.1.1]pentan-1- |
| | methyl-1H-pyrazol-1-yl)-1,3,4- | | yl)acetonitrile as the “amine |
| | thiadiazol-2-yl)-2-oxo-2H-pyran-6- | | reagent” |
| | carboxamide | | |
| 117 | 4-((3-(2-hydroxypropan-2- | 473.15 | Prepared according to Example 1, |
| | yl)bicyclo[1.1.1]pentan-1-yl)amino)- | | Part C, Step 2 using 2-(3- |
| | 3-methoxy-N-(5-(5-methyl-1H- | | aminobicyclo[1.1.1]pentan-1- |
| | pyrazol-1-yl)-1,3,4-thiadiazol-2-yl)-2- | | yl)propan-2-ol as the “amine |
| | oxo-2H-pyran-6-carboxamide | | reagent” |
| 118 | (R)-3-methoxy-N-(5-(5-methyl-1H- | 419.05 | Prepared according to Example 1, |
| | pyrazol-1-yl)-1,3,4-thiadiazol-2-yl)-4- | | Part C, Step 2 using (R)-oxetan-2- |
| | ((oxetan-2-ylmethyl)amino)-2-oxo- | | ylmethanamine as the “amine |
| | 2H-pyran-6-carboxamide | | reagent” |
| 119 | (R)-3-methoxy-4-((2- | 421.10 | Prepared according to Example 1, |
| | methoxypropyl)amino)-N-(5-(5- | | Part C, Step 2 using (R)-2- |
| | methyl-1H-pyrazol-1-yl)-1,3,4- | | methoxypropan-1-amine as the |
| | thiadiazol-2-yl)-2-oxo-2H-pyran-6- | | “amine reagent” |
| | carboxamide | | |
| 120 | 3-methoxy-4-((2-methoxy-2- | 435.15 | Prepared according to Example 13 |
| | methylpropyl)amino)-N-(5-(5-methyl- | | using 2-methoxy-2-methylpropan-1- |
| | 1H-pyrazol-1-yl)-1,3,4-thiadiazol-2- | | amine as the “amine reagent” |
| | yl)-2-oxo-2H-pyran-6-carboxamide | | |
| 121 | (R)-3-methoxy-4-((1-methoxypropan- | 421.1 | Prepared according to Example 13 |
| | 2-yl)amino)-N-(5-(5-methyl-1H- | | using (R)-1-methoxypropan-2-amine |
| | pyrazol-1-yl)-1,3,4-thiadiazol-2-yl)-2- | | as the “amine reagent” |
| | oxo-2H-pyran-6-carboxamide | | |
| 122* | 3-methoxy-4-(((1R,2S)-2- | 433.0 | Compounds 122* and 123* were |
| | methoxycyclobutyl)amino)-N-(5-(5- | | prepared and stereoisomers separated |
| | methyl-1H-pyrazol-1-yl)-1,3,4- | | according to Example 14 using rac- |
| | thiadiazol-2-yl)-2-oxo-2H-pyran-6- | | (1,2-cis)-2-methoxycyclobutan-1- |
| | carboxamide | | amine as the “amine reagent”. |
| 123* | 3-methoxy-4-(((1S,2R)-2- | 433.0 | Compound 122* was the first eluting |
| | methoxycyclobutyl)amino)-N-(5-(5- | | peak; Compound 123* was the |
| | methyl-1H-pyrazol-1-yl)-1,3,4- | | second eluting peak. |
| | thiadiazol-2-yl)-2-oxo-2H-pyran-6- | | |
| | carboxamide | | |
| 124* | 3-methoxy-4-(((1S,3R)-3- | 447.10 | Compounds 124* and 125* were |
| | methoxycyclopentyl)amino)-N-(5-(5- | | prepared and stereoisomers separated |
| | methyl-1H-pyrazol-1-yl)-1,3,4- | | according to Example 14 using 4- |
| | thiadiazol-2-yl)-2-oxo-2H-pyran-6- | | bromo-5-methoxy-N-[5-(5- |
| | carboxamide | | methylpyrazol-1-yl)-1,3,4-thiadiazol- |
| 125* | 3-methoxy-4-(((1R,3S)-3- | 447.10 | 2-yl]-6-oxopyran-2-carboxamide as |
| | methoxycyclopentyl)amino)-N-(5-(5- | | “halo-pyrone reagent” and rac-(1,3- |
| | methyl-1H-pyrazol-1-yl)-1,3,4- | | cis)-3-methoxycyclopentan-1-amine |
| | thiadiazol-2-yl)-2-oxo-2H-pyran-6- | | as the “amine reagent”. |
| | carboxamide | | Compound 124* was the first eluting |
| | | | peak; Compound 125* was the |
| | | | second eluting peak. |
| 127 | N-(5-(3-chloro-1-methyl-1H-pyrrol-2- | 447.0 | Prepared according to Example 10, |
| | yl)-1,3,4-thiadiazol-2-yl)-3-methoxy- | | Step 7 using 2-methoxyethan-1- |
| | 4-((2-methoxyethyl)amino)-2-oxo- | | amine as the “amine reagent” |
| | 2H-pyran-6-carboxamide | | |
| 128 | (R)-N-(5-(3-chloro-1-methyl-1H- | 453.95 | Prepared according to Example 10, |
| | pyrrol-2-yl)-1,3,4-thiadiazol-2-yl)-3- | | Step 7 using (R)-1-methoxypropan- |
| | methoxy-4-((1-methoxypropan-2- | | 2-amine as the “amine reagent” |
| | yl)amino)-2-oxo-2H-pyran-6- | | |
| | carboxamide | | |
| 143 | 4-((1,3-dimethoxypropan-2- | 467.0 | Prepared according to Example 32, |
| | yl)amino)-N-(5-(5-(hydroxymethyl)- | | Step 5 using 1,3-dimethoxypropan-2- |
| | 1H-pyrazol-1-yl)-1,3,4-thiadiazol-2- | | amine as the “amine reagent” |
| | yl)-3-methoxy-2-oxo-2H-pyran-6- | | |
| | carboxamide | | |
| 146 | N-[5-(3-chlorothiophen-2-yl)-1,3,4- | 442.9 | Prepared according to Example 4 by |
| | thiadiazol-2-yl]-5-methoxy-4-[(2- | | replacing 4-chlorothiophene-3- |
| | methoxyethyl)amino]-6-oxopyran-2- | | carbonitrile with 3-chlorothiophene- |
| | carboxamide | | 2-carbonitrile |
| 147 | N-[5-(3-chlorothiophen-2-yl)-1,3,4- | 428.90 | Prepared according to step 2 of |
| | thiadiazol-2-yl]-4-[(2- | | Example 21 by replacing 5-methoxy- |
| | hydroxyethyl)amino]-5-methoxy-6- | | 4-({3-methoxybicyclo[1.1.1]pentan- |
| | oxopyran-2-carboxamide | | 1-yl}amino)-N-[5-(5-methylpyrazol- |
| | | | 1-yl)-1,3,4-thiadiazol-2-yl]-6- |
| | | | oxopyran-2-carboxamide with N-[5- |
| | | | (3-chlorothiophen-2-yl)-1,3,4- |
| | | | thiadiazol-2-yl]-5-methoxy-4-[(2- |
| | | | methoxyethyl)amino]-6-oxopyran-2- |
| | | | carboxamide (Compound 146) |
| 151 | 5-methoxy-4-[(3S)-3-methoxy-2- | 447.1 | Prepared according to Example 37 |
| | oxopyrrolidin-1-yl]-N-[5-(5- | | by replacing (R)-3- |
| | methylpyrazol-1-yl)-1,3,4-thiadiazol- | | hydroxypyrrolidin-2-one with (S)-3- |
| | 2-yl]-6-oxopyran-2-carboxamide | | hydroxypyrrolidin-2-one |
| 154 | (R)-3-methoxy-4-(4-methoxy-2- | 447.1 | Prepared according to Example 37 |
| | oxopyrrolidin-1-yl)-N-(5-(5-methyl- | | by replacing (R)-3- |
| | 1H-pyrazol-1-yl)-1,3,4-thiadiazol-2- | | hydroxypyrrolidin-2-one with (R)-4- |
| | yl)-2-oxo-2H-pyran-6-carboxamide | | hydroxypyrrolidin-2-one |
| 155 | (S)-3-methoxy-4-(4-methoxy-2- | 447.1 | Prepared according to Example 37 |
| | oxopyrrolidin-1-yl)-N-(5-(5-methyl- | | by replacing (R)-3- |
| | 1H-pyrazol-1-yl)-1,3,4-thiadiazol-2- | | hydroxypyrrolidin-2-one with (S)-4- |
| | yl)-2-oxo-2H-pyran-6-carboxamide | | hydroxypyrrolidin-2-one |
| 161 | 5-methoxy-4-{[(2R,3R)-3- | 435.1 | Prepared according to Example 43 |
| | methoxybutan-2-yl]amino}-N-[5-(5- | | by replacing (3S,4S)-4-aminooxolan- |
| | methylpyrazol-1-yl)-1,3,4-thiadiazol- | | 3-ol with (2R,3R)-3-aminobutan-2-ol |
| | 2-yl]-6-oxopyran-2-carboxamide | | |
| 162 | 5-methoxy-4-{[(3R,4R)-4- | 449.10 | Prepared according to Example 43 |
| | methoxyoxolan-3-yl]amino}-N-[5-(5- | | by replacing (3S,4S)-4-aminooxolan- |
| | methylpyrazol-1-yl)-1,3,4-thiadiazol- | | 3-ol with (3R,4R)-4-aminooxolan-3- |
| | 2-yl]-6-oxopyran-2-carboxamide | | ol |
| 163 | 5-methoxy-4-{[(2S,3S)-3- | 435.05 | Prepared according to Example 43 |
| | methoxybutan-2-yl]amino}-6-oxo-N- | | by replacing (3S,4S)-4-aminooxolan- |
| | [5-(pyrazol-1-yl)-1,3,4-thiadiazol-2- | | 3-ol with (2S,3S)-3-aminobutan-2-ol |
| | yl]pyran-2-carboxamide | | |
| Rac-164 | 3-methoxy-4-(((1R,2S)-2-methoxy- | 495.20 | Prepared according to Example 43 |
| | 2,3-dihydro-1H-inden-1-yl)amino)-N- | | by replacing (3S,4S)-4-aminooxolan- |
| | (5-(5-methyl-1H-pyrazol-1-yl)-1,3,4- | | 3-ol with rac-(1,2-cis)-1-amino-2,3- |
| | thiadiazol-2-yl)-2-oxo-2H-pyran-6- | | dihydro-1H-inden-2-ol |
| | carboxamide (Compound 164a*) | | |
| | 3-methoxy-4-(((1S,2R)-2-methoxy- | | |
| | 2,3-dihydro-1H-inden-1-yl)amino)-N- | | |
| | (5-(5-methyl-1H-pyrazol-1-yl)-1,3,4- | | |
| | thiadiazol-2-yl)-2-oxo-2H-pyran-6- | | |
| | carboxamide (Compound 164b*) | | |
| 169 | 5-methoxy-N-[5-(5-methylpyrazol-1- | 463.1 | Prepared according to Example 47 |
| | yl)-1,3,4-thiadiazol-2-yl]-6-oxo-4- | | by replacing cyclopropanol with (R)- |
| | ({2-[(3R)-oxolan-3- | | tetrahydrofuran-3-ol |
| | yloxy]ethyl}amino)pyran-2- | | |
| | carboxamide | | |
| 170 | (R)-3-methoxy-4-((1-methoxy-3- | 497.15 | Prepared according to Example 43 |
| | phenylpropan-2-yl)amino)-N-(5-(5- | | by replacing (3S,4S)-4-aminooxolan- |
| | methyl-1H-pyrazol-1-yl)-1,3,4- | | 3-ol with (R)-2-amino-3- |
| | thiadiazol-2-yl)-2-oxo-2H-pyran-6- | | phenylpropan-1-ol |
| | carboxamide | | |
| 175 | (S)-4-(2-(cyanomethyl)pyrrolidin-1- | 442.1 | Prepared according to Example 50 |
| | yl)-3-methoxy-N-(5-(5-methyl-1H- | | by replacing (R)-prolinol with (S)- |
| | pyrazol-1-yl)-1,3,4-thiadiazol-2-yl)-2- | | prolinol |
| | oxo-2H-pyran-6-carboxamide | | |
| 176 | 4-((2-cyanoethyl)amino)-3-methoxy- | 402.05 | Prepared according to Example 50 |
| | N-(5-(5-methyl-1H-pyrazol-1-yl)- | | by replacing (R)-prolinol with 2- |
| | 1,3,4-thiadiazol-2-yl)-2-oxo-2H- | | aminoethan-1-ol |
| | pyran-6-carboxamide | | |
| 179 | 4-(isopropylamino)-5-methoxy-N-[5- | 391.10 | Prepared according to Example 17, |
| | (5-methylpyrazol-1-yl)-1,3,4- | | using propan-2-amine as the “amine |
| | thiadiazol-2-yl]-6-oxopyran-2- | | reagent” |
| | carboxamide | | |
| 180 | 4-(cyclopropylamino)-5-methoxy-N- | 389.05 | Prepared according to Example 1, |
| | [5-(5-methylpyrazol-1-yl)-1,3,4- | | Part C, Step 2 using |
| | thiadiazol-2-yl]-6-oxopyran-2- | | cyclopropanamine as the “amine |
| | carboxamide | | reagent” |
| 181 | 4-((2-(dimethylamino)ethyl)amino)- | 420.20 | Prepared according to Example 1, |
| | 3-methoxy-N-(5-(5-methyl-1H- | | Part C, Step 2 using N,N- |
| | pyrazol-1-yl)-1,3,4-thiadiazol-2-yl)-2- | | dimethylethane-1,2-diamine as the |
| | oxo-2H-pyran-6-carboxamide | | “amine reagent” |
| 182 | 4-(bicyclo[1.1.1]pentan-1-ylamino)- | 415.05 | Prepared according to Example 1, |
| | 3-methoxy-N-(5-(5-methyl-1H- | | Part C, Step 2 using 3- |
| | pyrazol-1-yl)-1,3,4-thiadiazol-2-yl)-2- | | methylbicyclo[1.1.1]pentan-1-amine |
| | oxo-2H-pyran-6-carboxamide | | as the “amine reagent” |
| 183 | 3-methoxy-N-(5-(5-methyl-1H- | 455.10 | Prepared according to Example 1, |
| | pyrazol-1-yl)-1,3,4-thiadiazol-2-yl)-4- | | Part C, Step 2 using 2- |
| | (2-(methylsulfonyl)ethyl)amino)-2- | | (methylsulfonyl)ethan-1-amine as the |
| | oxo-2H-pyran-6-carboxamide | | amine reagent” |
| 184 | (R)-3-methoxy-N-(5-(5-methyl-1H- | 419.10 | Prepared according to Example 1, |
| | pyrazol-1-yl)-1,3,4-thiadiazol-2-yl)-2- | | Part C, Step 2 using (R)- |
| | oxo-4-((tetrahydrofuran-3-yl)amino)- | | tetrahydrofuran-3-amine as the |
| | 2H-pyran-6-carboxamide | | amine reagent” |
| 185 | (S)-3-methoxy-N-(5-(5-methyl-1H- | 419.05 | Prepared according to Example 1, |
| | pyrazol-1-yl)-1,3,4-thiadiazol-2-yl)-2- | | Part C, Step 2 using (S)- |
| | oxo-4-((tetrahydrofuran-3-yl)amino)- | | tetrahydrofuran-3-amine as the |
| | 2H-pyran-6-carboxamide | | “amine reagent” |
| 186 | 3-methoxy-N-(5-(5-methyl-1H- | 419.05 | Prepared according to Example 1, |
| | pyrazol-1-yl)-1,3,4-thiadiazol-2-yl)-4- | | Part C, Step 2 using morpholine as |
| | morpholino-2-oxo-2H-pyran-6- | | the “amine reagent” |
| | carboxamide | | |
| 187 | (S)-3-methoxy-4-((2- | 421.10 | Prepared according to Example 1, |
| | methoxypropyl)amino)-N-(5-(5- | | Part C, Step 2 using (S)-2- |
| | methyl-1H-pyrazol-1-yl)-1,3,4- | | methoxypropan-1-amine as the |
| | thiadiazol-2-yl)-2-oxo-2H-pyran-6- | | “amine reagent” |
| | carboxamide | | |
| Rac-188 | N-(5-(3-chloro-1-methyl-1H-pyrrol-2- | 466.05 | Prepared according to Example 10 |
| | yl)-1,3,4-thiadiazol-2-yl)-4-(((1S,2R)- | | Step 7 using rac-(1,2-cis)-2- |
| | 2-hydroxycyclopentyl)amino)-3- | | aminocyclopentan-1-ol as the |
| | methoxy-2-oxo-2H-pyran-6- | | “amine reagent” |
| | carboxamide (Compound 188a*) | | |
| | N-(5-(3-chloro-1-methyl-1H-pyrrol-2- | | |
| | yl)-1,3,4-thiadiazol-2-yl)-4-(((1R,2S)- | | |
| | 2-hydroxycyclopentyl)amino)-3- | | |
| | methoxy-2-oxo-2H-pyran-6- | | |
| | carboxamide (Compound 188b*) | | |
| Rac-189 | 4-(((1S,2R)-2- | 447.1 | Prepared according to Example 1, |
| | hydroxycyclohexyl)amino)-3- | | Part C, Step 2 using rac-(1,2-cis)-2- |
| | methoxy-N-(5-(5-methyl-1H-pyrazol- | | aminocyclohexan-1-ol as the “amine |
| | 1-yl)-1,3,4-thiadiazol-2-yl)-2-oxo- | | reagent” |
| | 2H-pyran-6-carboxamide (Compound | | |
| | 189a*) | | |
| | 4-(((1R,2S)-2- | | |
| | hydroxycyclohexyl)amino)-3- | | |
| | methoxy-N-(5-(5-methyl-1H-pyrazol- | | |
| | 1-yl)-1,3,4-thiadiazol-2-yl)-2-oxo- | | |
| | 2H-pyran-6-carboxamide (Compound | | |
| | 189b*) | | |
| 190 | 3-methoxy-4-(((1- | 433.05 | Prepared according to Example 1, |
| | methoxycyclopropyl)methyl)amino)- | | Part C, Step 2 using (1- |
| | N-(5-(5-methyl-1H-pyrazol-1-y])- | | methoxycyclopropy 1)methanamine |
| | 1,3,4-thiadiazol-2-yl)-2-oxo-2H- | | as the “amine reagent” |
| | pyran-6-carboxamide | | |
| 193 | (S)-N-(5-(3-chloro-1-methyl-1H- | 454.10 | Prepared according to Example 1, |
| | pyrrol-2-yl)-1,3,4-thiadiazol-2-yl)-3- | | Part C, Step 2 using (S)-2- |
| | methoxy-4-((2- | | methoxypropan-1-amine as the |
| | methoxypropyl)amino)-2-oxo-2H- | | “amine reagent” |
| | pyran-6-carboxamide | | |
| 194 | (R)-3-methoxy-4-(2- | 433.1 | Prepared according to Example 1, |
| | (methoxymethyl)azetidin-1-yl)-N-(5- | | Part C, Step 2 using (R)-2- |
| | (5-methyl-1H-pyrazol-1-yl)-1,3,4- | | (methoxymethyl)azetidine as the |
| | thiadiazol-2-yl)-2-oxo-2H-pyran-6- | | “amine reagent” |
| | carboxamide | | |
| 195 | 4-((2,2-dimethyl-1,3-dioxan-5- | 461.15 | Prepared according to Example 1, |
| | yl)amino)-3-methoxy-N-(5-(5- | | Part C, Step 2 using 2,2-dimethyl- |
| | methyl-1H-pyrazol-1-yl)-1,3,4- | | 1,3-dioxan-5-amine as the “amine |
| | thiadiazol-2-yl)-2-oxo-2H-pyran-6- | | reagent” |
| | carboxamide | | |
| 196 | 3-methoxy-4-((1- | 447.10 | Prepared according to Example 1, |
| | (methoxymethyl)cyclobutyl)amino)- | | Part C, Step 2 using 1- |
| | N-(5-(5-methyl-1H-pyrazol-1-y])- | | (methoxymethyl)cyclobutan-1-amine |
| | 1,3,4-thiadiazol-2-yl)-2-oxo-2H- | | as the “amine reagent” |
| | pyran-6-carboxamide | | |
| 197 | N-(5-(3-chloro-1-methyl-1H-pyrrol-2- | 466.10 | Prepared according to Example 10 |
| | yl)-1,3,4-thiadiazol-2-yl)-4-(((1R,2S)- | | Step 7 (1S,2R)-2-aminocyclopentan- |
| | 2-hydroxycyclopentyl)amino)-3- | | 1-ol as the “amine reagent” |
| | methoxy-2-oxo-2H-pyran-6- | | |
| | carboxamide | | |
| 202 | 3-methoxy-4-(((1R,2R)-2- | 462.20 | Prepared according to Example 1, |
| | methoxycyclohexyl)amino)-N-(5-(5- | | Part C, Step 2 using (1R,2R)-2- |
| | methyl-1H-pyrazol-1-yl)-1,3,4- | | methoxycyclohexan-1-amine as the |
| | thiadiazol-2-yl)-2-oxo-2H-pyran-6- | | “amine reagent” |
| | carboxamide | | |
| 204 | 3-methoxy-N-(5-(5-methyl-1H- | 406.15 | Prepared according to Example 55 |
| | pyrazol-1-yl)-1,3,4-thiadiazol-2-yl)-4- | | by replacing tert-butyl (2- |
| | ((2-(methylamino)ethyl)amino)-2- | | aminoethyl)carbamate with tert-butyl |
| | oxo-2H-pyran-6-carboxamide | | (2-aminoethyl)(methyl)carbamate |
| 206 | 4-(((1S,2R)-2-(2,2- | 497.15 | Prepared according to Example 48 |
| | difluoroethoxy)cyclopentyl)amino)-3- | | by replacing (1S,2R)-2- |
| | methoxy-N-(5-(5-methyl-1H-pyrazol- | | aminocyclopentan-1-ol |
| | 1-yl)-1,3,4-thiadiazol-2-yl)-2-oxo- | | hydrochloride with (1R,2S)-2- |
| | 2H-pyran-6-carboxamide | | aminocyclopentan-1-ol |
| | | | hydrochloride |
| 207 | 3-methoxy-4-(((2S,3R)-3- | 435.15 | Prepared according to Example 43 |
| | methoxybutan-2-yl)amino)-N-(5-(5- | | by replacing (3S,4S)-4-aminooxolan- |
| | methyl-1H-pyrazol-1-yl)-1,3,4- | | 3-ol with (2R,3S)-3-aminobutan-2-ol |
| | thiadiazol-2-yl)-2-oxo-2H-pyran-6- | | |
| | carboxamide | | |
| 213 | (R)-N-(5-(3-chloro-1-methyl-1H- | 516.1 | Prepared according to Example 10 |
| | pyrrol-2-yl)-1,3,4-thiadiazol-2-yl)-3- | | Step 7 by replacing propan-2-amine |
| | methoxy-4-((2-methoxy-1- | | with (R)-2-methoxy-1-phenylethan- |
| | phenylethyl)amino)-2-oxo-2H-pyran- | | 1-amine |
| | 6-carboxamide | | |
| 216 | 3-methoxy-N-(5-(5-methyl-1H- | 403.10 | Prepared according to Example 1, |
| | pyrazol-1-yl)-1,3,4-thiadiazol-2-yl)-2- | | Part C, Step 2 using pyrrolidine as |
| | oxo-4-(pyrrolidin-1-yl)-2H-pyran-6- | | the “amine reagent” |
| | carboxamide | | |
| 217 | 4-(trans-4- | 447.00 | Prepared according to Example 1, |
| | hydroxycyclohexyl)amino)-3- | | Part C, Step 2 using (1,4-trans)-4- |
| | methoxy-N-(5-(5-methyl-1H-pyrazol- | | aminocyclohexan-1-ol as the “amine |
| | 1-yl)-1,3,4-thiadiazol-2-yl)-2-oxo- | | reagent” |
| | 2H-pyran-6-carboxamide | | |
| 218 | 4-((3- | 445.10 | Prepared according to Example 1, |
| | (hydroxymethyl)bicyclo[1.1.1]pentan- | | Part C, Step 2 using (3- |
| | 1-yl)amino)-3-methoxy-N-(5-(5- | | aminobicyclo[1.1.1]pentan-1- |
| | methyl-1H-pyrazol-1-yl)-1,3,4- | | yl)methanol as the “amine reagent” |
| | thiadiazol-2-yl)-2-oxo-2H-pyran-6- | | |
| | carboxamide | | |
| 219 | 3-methoxy-4-((1,4-cis-4- | 461.00 | Prepared according to Example 1, |
| | methoxycyclohexyl)amino)-N-(5-(5- | | Part C, Step 2 using (1,4-cis)-4- |
| | methyl-1H-pyrazol-1-yl)-1,3,4- | | methoxycyclohexan-1-amine as the |
| | thiadiazol-2-yl)-2-oxo-2H-pyran-6- | | “amine reagent” |
| | carboxamide | | |
| 220 | 4-(1,4-cis-4- | 447.10 | Prepared according to Example 1, |
| | hydroxycyclohexyl)amino)-3- | | Part C, Step 2 using (1,4-cis)-4- |
| | methoxy-N-(5-(5-methyl-1H-pyrazol- | | aminocyclohexan-1-ol as the “amine |
| | 1-yl)-1,3,4-thiadiazol-2-yl)-2-oxo- | | reagent” |
| | 2H-pyran-6-carboxamide | | |
| 222 | (R)-1-(1-methoxypropan-2-yl)-N-(5- | 433.0 | Prepared according to Example 64 |
| | (5-methyl-1H-pyrazol-1-yl)-1,3,4- | | with the following modifications: |
| | thiadiazol-2-yl)-5-oxo-1,2,3,5- | | (2S)-1-methoxypropan-2-amine was |
| | tetrahydropyrano[3,4-6][1,4]oxazine- | | used as the “amine reagent” in Step |
| | 7-carboxamide | | 4. In Step 7, no triethylamine was |
| | | | used and the reaction was performed |
| | | | at 80° C. for 1.5 h. |
| 223 | 1-((1R,2S)-2-cyanocyclopentyl)-N-(5- | 454.0 | Prepared according to Example 64 |
| | (5-methyl-1H-pyrazol-1-yl)-1,3,4- | | with the following modifications: |
| | thiadiazol-2-yl)-5-oxo-1,2,3,5- | | ((1R,2S)-2-aminocyclopentane-1- |
| | tetrahydropyrano[3,4-b][1,4]oxazine- | | carbonitrile was used as the “amine |
| | 7-carboxamide | | reagent” in Step 4. In Step 7, no |
| | | | triethylamine was used and the |
| | | | second stage purification was |
| | | | performed using Chiral-Prep-HPLC |
| | | | with the following conditions: |
| | | | CHIRAL ART Amylose-SA, 2*25 |
| | | | cm, 5 um; mobile phase, Hexanes |
| | | | (0.1% TFA) and MeOH:EtOH = 1:1 |
| | | | (hold 70% MeOH:EtOH = 1:1 in 7 |
| | | | min); Detector, UV 254 nm. |
| 224 | 1-((1S,2R)-2-cyanocyclopentyl)-N-(5- | 454.0 | Prepared according to Example 64 |
| | (5-methyl-1H-pyrazol-1-yl)-1,3,4- | | with the following modifications: |
| | thiadiazol-2-yl)-5-oxo-1,2,3,5- | | (1S,2R)-2-aminocyclopentane-1- |
| | tetrahydropyrano[3,4-b][1,4]oxazine- | | carbonitrile was used as the “amine |
| | 7-carboxamide | | reagent” in Step 4. In Step 7, no |
| | | | triethylamine was used and the |
| | | | second stage purification was |
| | | | performed using Chiral-Prep-HPLC |
| | | | with the following conditions: |
| | | | CHIRAL ART Amylose-SA, 2*25 |
| | | | cm, 5 um; mobile phase, Hexanes |
| | | | (0.1% TFA) and MeOH:EtOH = 1:1 |
| | | | (hold 70% MeOH:EtOH = 1:1 in 7 |
| | | | min); Detector, UV 254 nm. |
| 225 | (R)-1-(1-cyclopropyl-2- | 459.2 | Prepared according to Example 64 |
| | methoxyethyl)-N-(5-(5-methyl-1H- | | with the following modifications: |
| | pyrazol-1-yl)-1,3,4-thiadiazol-2-yl)-5- | | (1R)-1-cyclopropyl-2- |
| | oxo-1,2,3,5-tetrahydropyrano[3,4- | | methoxyethanamine was used as the |
| | b][1,4]oxazine-7-carboxamide | | “amine reagent” in Step 4. In Step |
| | | | 7, no triethylamine was used and the |
| | | | reaction was performed at 80° C. for |
| | | | 1 h. |
| 226 | (S)-1-(1-cyclopropyl-2- | 459.2 | Prepared according to Example 64 |
| | methoxyethyl)-N-(5-(5-methyl-1H- | | with the following modifications: |
| | pyrazol-1-yl)-1,3,4-thiadiazol-2-yl)-5- | | (1S)-1-cyclopropyl-2- |
| | oxo-1,2,3,5-tetrahydropyrano[3,4- | | methoxyethanamine was used as the |
| | b][1,4]oxazine-7-carboxamide | | “amine reagent” in Step 4. In Step |
| | | | 7, no triethylamine was used and the |
| | | | reaction was performed at 80° C. for |
| | | | 1 h. |
| 227 | (R)-1-(2-methoxy-1-phenylethyl)-N- | 495.2 | Prepared according to Example 64 |
| | (5-(5-methyl-1H-pyrazol-1-yl)-1,3,4- | | with the following modifications: |
| | thiadiazol-2-yl)-5-oxo-1,2,3,5- | | (1R)-2-methoxy-1-phenylethanamine |
| | tetrahydropyrano[3,4-6][1,4]oxazine- | | was used as the “amine reagent” in |
| | 7-carboxamide | | Step 4. In Step 7, no triethylamine |
| | | | was used and the reaction was |
| | | | performed at 50° C. for 5 h. |
| 228 | 4-((2-methoxyethyl)amino)-N-(5-(5- | — | Compound 228 may be prepared |
| | methyl-1H-pyrazol-1-yl)-1,3,4- | | according to Example 15 using 2- |
| | thiadiazol-2-yl)-3-(2- | | morpholinoethan-1-ol in place of 2- |
| | morpholinoethoxy)-2-oxo-2H-pyran- | | methoxyethanol in step 1 and using |
| | 6-carboxamide | | 2-methoxyethan-1-amine in place of |
| | | | (R)-2-methoxy-1-phenylethan-1- |
| | | | amine as the “amine reagent” in |
| | | | step 4. |
| 229 | 4-((2-methoxyethyl)amino)-N-(5-(5- | — | Compound 229 may be prepared |
| | methyl-1H-pyrazol-1-yl)-1,3,4- | | according to Example 15 using 2- |
| | thiadiazol-2-yl)-2-oxo-3-(2- | | thiomorpholinoethan-1-ol in place of |
| | thiomorpholinoethoxy)-2H-pyran-6- | | 2-methoxyethanol in step 1 and using |
| | carboxamide | | 2-methoxyethan-1-amine in place of |
| | | | (R)-2-methoxy-1-phenylethan-1- |
| | | | amine as the “amine reagent” in |
| | | | step 4. |
| 230 | 3-((1-methoxycyclopropyl)methoxy)- | — | Compound 230 may be prepared |
| | 4-((2-methoxyethyl)amino)-N-(5-(5- | | according to Example 15 using (1- |
| | methyl-1H-pyrazol-1-yl)-1,3,4- | | methoxycyclopropyl)methanol in |
| | thiadiazol-2-yl)-2-oxo-2H-pyran-6- | | place of 2-methoxyethanol in step 1 |
| | carboxamide | | and using 2-methoxyethan-1-amine |
| | | | in place of (R)-2-methoxy-1- |
| | | | phenylethan-1-amine as the “amine |
| | | | reagent” in step 4. |
| 231 | (S)-4-((2-methoxyethyl)amino)-N-(5- | — | Compound 231 may be prepared |
| | (5-methyl-1H-pyrazol-1-yl)-1,3,4- | | according to Example 15 using (R)- |
| | thiadiazol-2-yl)-2-oxo-3- | | tetrahydrofuran-3-ol in place of 2- |
| | ((tetrahydrofuran-3-yl)oxy)-2H- | | methoxyethanol in step 1 and using |
| | pyran-6-carboxamide | | 2-methoxyethan-1-amine in place of |
| | | | (R)-2-methoxy-1-phenylethan-1- |
| | | | amine as the “amine reagent” in |
| | | | step 4. |
| 232 | (R)-4-((2-methoxyethyl)amino)-N-(5- | — | Compound 232 may be prepared |
| | (5-methyl-1H-pyrazol-1-yl)-1,3,4- | | according to Example 15 using (S)- |
| | thiadiazol-2-yl)-2-oxo-3- | | tetrahydrofuran-3-ol in place of 2- |
| | ((tetrahydrofuran-3-yl)oxy)-2H- | | methoxyethanol in step 1 and using |
| | pyran-6-carboxamide | | 2-methoxyethan-1-amine in place of |
| | | | (R)-2-methoxy-1-phenylethan-1- |
| | | | amine as the “amine reagent” in |
| | | | step 4. |

### Biological Assay Methods

cGAS catalyzes the cyclization of ATP and GTP to produce cGAMP, which is the activating ligand of STING. Human cGAS (hcGAS) inhibition can thus be quantified by measuring how much cGAMP is formed either indirectly, by monitoring ATP-depletion as in the hcGAS Kinase-Glo assay (as described below), or directly, such as using the hcGAS LCMS assay (as described below).

(i) hcGAS Kinase-Glo Assay

Compounds were tested for their human-cGAS (11-cGAS) inhibition activity using the methodology reported in Lama et al., “Development of human cGAS-specific small molecule inhibitors for repression of dsDNA-triggered interferon expression”, Nature Communications 10, Article number: 2261 (2019), with slight changes to some conditions as shown in Table B.

The results of the assay, expressed in IC₅₀ values, are reported in Table D with the following designations: A represents an IC₅₀ value<0.01 μM; B represents an IC₅₀ value≥0.01 μM and <0.03 μM; C represents an IC₅₀ value≥0.03 μM and <0.1 μM; D represents an IC₅₀ value≥0.1.

TABLE B
Summary of hcGAS Kinase-Glo assay conditions

| | | Lama | Changes |
|---|---|---|---|
| Reagent/Conditions | | et al. | to Lama |
| Enzyme | hcGAS (nM) | 100 | 40 |
| Buffer | Tris(hydroxymethyl)aminomethane | 20 | 20 |
| | hydrochloride (Tris-HCl) pH 7.4 (mM) | | |
| | MgCl₂ (mM) | 5 | 10 |
| | NaCl (mM) | 150 | 25 |
| | Tween ™-20 (%) | 0.01 | 0.01 |
| | ZnCl₂ (μM) | 1 | 1 |
| | Dithiothreitol (DTT) (mM) | 1 | 1 |
| | Dimethylsulfoxide (DMSO) (%) | 0.5 | 5 |
| Substrates | Adenosine triphosphate (ATP) (μM) | 100 | 100 |
| | Guanosine triphosphate (GTP) (μM) | 100 | 100 |
| | Double stranded DNA (dsDNA) (nM) | 25 | 25 |
| Assay | Plate (wells) | 384 | 384 |
| | Incubation length (h) | 7 | 3 |
| | Total volume (μL) | 20 | 20 |
| | Kinase-Glo Max (μL) | 20 | 20 |

(ii) hcGAS LCMS Assay

Compounds were tested for their h-cGAS inhibition activity using direct measurement of cGAMP production by LC/MS. Briefly, compounds were incubated with enzyme and substrates for 4 hours (see Table C), before the reaction was stopped with 3 volumes of 70/30 Acetonitrile/H₂O mix containing 0.15 mM cGAMP-¹³C₁₀⁵N₅ as internal standard and mixed for min. After centrifugation (3700 rpm, 10 min, 10° Celsius), 100 mL of each reaction was collected and mixed to equal volume of Acetonitrile. Samples were then analyzed on Qexective (Column: XBridge BEH Amide Column, 130 Å, 3.5 μm, 3 mm×50 mm, Mobile phase A: 10 mM Ammonium acetate in 95/5% H₂O/Acetonitrile (0.5% DMSO), Mobile phase B: Acetonitrile (0.5% DMSO).

The results of the assay, expressed as IC₅₀ values, are reported in Table D with the following designations: A represents an IC₅₀ value<0.005 μM; B represents an IC₅₀ value≥0.005 μM and <0.02 μM, C represents an IC₅₀ value≥0.02 μM and <0.06 μM; D represents an IC₅₀ value≥0.06 and <0.2 μM; E represents an IC₅₀ value≥0.2.

TABLE C
Summary of heGAS LCMS assay conditions

| | | |
|---|---|---|
| Enzyme | hcGAS (nM) | 0.5 |
| Buffer | Tris(hydroxymethyl)aminomethane | 20 |
| | hydrochloride (Tris-HCl) pH 7.4 (mM) | |
| | MgCl₂ (mM) | 10 |
| | NaCl (mM) | 25 |
| | Tween ™-20 (%) | 0.01 |
| | ZnCl₂ (μM) | 1 |
| | Dithiothreitol (DTT) (mM) | 1 |
| | Dimethylsulfoxide (DMSO) (%) | 5 |
| Substrates | ATP (μM) | 100 |
| | Guanosine triphosphate (GTP) (μM) | 100 |
| | Double stranded DNA (dsDNA) (nM) | 25 |
| Assay | Plate (wells) | 96 |
| | Incubation length (h) | 4 |
| | Total volume (μL) | 40 |

(iii) Results

Activity data obtained from the above referenced assay methods is provided in Table D. Dashed lines (--) indicate no data is available.

TABLE D
Activity Data

| | hcGAS | hcGAS | | hcGAS | hcGAS |
|---|---|---|---|---|---|
| | kinase- | LCMS | | kinase- | LCMS |
| | glo | IC₅₀ | | glo | IC₅₀ |
| Comp'd | IC₅₀ (uM) | (uM) | Comp'd | IC₅₀ (uM) | (uM) |
| 1 | B | B | 118 | — | B |
| 2 | B | — | 119 | B | B |
| 3* | — | A | 120 | B | B |
| 4* | — | B | 121 | B | B |
| 5 | — | B | 122* | — | B |
| Rac-6 | B | B | 123* | — | B |
| Rac-7 | A | A | 124* | — | B |
| 8 | B | B | 125* | — | B |
| 9 | C | — | 126 | — | B |
| 10 | B | B | 126-OMe | B | B |
| 11 | B | C | 127 | B | A |
| 12 | B | — | 128 | — | A |
| 13 | — | B | 129 | A | B |
| Rac-14 | B | B | 129-OMe | B | A |
| Rac-15 | C | — | 130 | C | C |
| 16 | C | — | 131 | B | B |
| 17 | — | C | 132* | — | B |
| 18 | — | A | 133* | — | B |
| 19 | B | — | 134 | C | — |
| 20 | — | C | 135 | — | C |
| 21* | — | B | 136 | B | B |
| 22* | — | A | 137 | D | — |
| Rac-23 | B | A | 138 | — | E |
| 24 | — | — | 139 | C | — |
| 25 | C | — | 140 | — | D |
| Rac-26 | — | A | 141 | D | — |
| 27 | — | B | 142 | — | C |
| Rac-28 | — | E | 143 | — | C |
| 29 | C | — | 144 | B | — |
| 30 | B | B | 145 | — | E |
| 31 | B | B | 146 | B | B |
| 32 | C | — | 147 | D | — |
| 33 | B | C | 148 | D | — |
| 34 | C | — | 149 | — | B |
| 35 | B | — | 150 | — | B |
| Rac-36 | B | B | 151 | — | B |
| Rac-37 | B | — | 152 | — | B |
| 38 | B | B | Rac-153 | — | B |
| 39 | B | — | 154 | — | C |
| 40 | B | — | 155 | — | B |
| 41 | A | A | 156 | — | D |
| 42 | A | A | 157 | — | E |
| Rac-43 | B | B | 158 | — | D |
| 44 | C | — | Rac-159 | — | B |
| 45 | B | — | 160 | — | B |
| 46 | B | — | 161 | — | A |
| 47* | — | A | 162 | — | B |
| 48* | — | B | 163 | — | D |
| 49 | — | C | Rac-164 | — | B |
| 50 | — | D | 165 | — | D |
| 51 | — | B | 166 | — | A |
| 52 | — | B | Rac-167 | — | A |
| 53 | — | A | 168 | — | D |
| 54 | — | A | 169 | — | E |
| Rac-55 | — | E | 170 | — | B |
| Rac-56 | — | B | 171 | — | B |
| 57 | B | B | 172* | — | B |
| 58 | B | — | 173* | — | C |
| 59 | B | B | 174 | — | C |
| 60 | B | B | 174-OH | C | — |
| Rac-61 | B | B | 175 | — | B |
| 62 | B | B | 176 | B | B |
| 63 | D | — | 177 | — | A |
| 64 | C | C | 177-Ac—OtBu | — | — |
| 65 | C | — | 178 | — | C |
| 66 | B | B | 178-Ac | — | — |
| Rac-67 | B | B | 179 | C | B |
| Rac-68 | B | B | 180 | C | — |
| 69 | B | — | 181 | D | — |
| 70 | — | B | 182 | B | B |
| 71* | — | B | 183 | C | B |
| 72* | — | B | 184 | C | C |
| 73 | — | A | 185 | C | B |
| 74 | — | A | 186 | C | — |
| 75 | C | B | 187 | B | B |
| 76 | B | B | Rac-188 | A | A |
| 77 | — | B | Rac-189 | B | B |
| 78 | — | A | 190 | — | B |
| 79* | — | D | 191* | — | C |
| 80* | — | E | 192* | — | B |
| Rac-81 | — | E | 193 | — | A |
| 82 | C | — | 194 | — | B |
| 83 | — | A | 195 | — | B |
| 84 | — | A | 196 | — | B |
| 85 | — | B | 197 | — | B |
| 86 | — | A | 198* | — | C |
| 87 | C | — | 199* | — | A |
| 88 | B | — | 200 | — | A |
| 89 | B | — | 201 | — | A |
| 90 | B | — | 202 | — | B |
| Rac-91 | A | B | 203 | D | — |
| Rac-92 | B | B | 204 | D | — |
| 93 | — | C | 205 | — | B |
| 94 | — | B | 205-Ac | — | — |
| 95 | — | B | 206 | — | B |
| 96 | — | A | 207 | — | C |
| 97 | — | B | 208 | — | B |
| 98 | — | B | Rac-209 | — | B |
| 99 | — | C | 210 | — | B |
| 100* | — | B | 210-Ac | — | — |
| 101* | — | B | 211 | — | B |
| 102 | — | B | 212 | — | B |
| 103 | C | B | 213 | — | A |
| 104 | C | — | 214* | — | B |
| 105 | C | — | 215* | — | B |
| 106 | C | C | 216 | C | B |
| Rac-107 | A | B | 217 | B | — |
| Rac-108 | B | B | 218 | B | — |
| Rac-109 | B | B | 219 | B | — |
| 110 | B | B | 220 | B | B |
| 111 | B | B | 223 | — | B |
| 112 | A | A | 224 | — | A |
| 111 | B | B | 225 | — | A |
| 112 | A | A | 226 | — | A |
| 113 | B | — | 227 | — | A |
| 114 | A | A | 228 | — | — |
| 115 | B | — | 229 | — | — |
| 116 | B | B | 230 | — | — |
| 117 | B | B | 231A | — | — |
| | | | 231B | — | — |

### OTHER EMBODIMENTS

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims or embodiments is introduced into another claim or embodiment. For example, any claim that is dependent on another claim can be modified to include one or more limitations found in any other claim that is dependent on the same base claim. Where the present disclosure recites elements presented as lists. e.g., in Markush group format, each subgroup of the elements is also disclosed, and any element(s) can be removed from the group. It should be further understood that when any variable (e.g., an R group) is present more than one time in a given Markush structure (e.g., 2 or more times), and that variable may be selected from a given list of two or more elements, unless otherwise stated or understood within the context of the present disclosure, that variable (e.g., the R group) at each repeated occurrence (e.g., 2 or more times) is independent of each other, being independently selected from that given list.

It should also be understood that, in general, where the present disclosure, or aspects of the present disclosure, is/are referred to as comprising particular elements and/or features, certain embodiments of the present disclosure or aspects of the present disclosure also consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are understood to be included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the present disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

The scope of the present embodiments described herein is not intended to be limited to the above Description, but also includes that as set forth in the appended claims. Those of ordinary skill in the art will appreciate that various changes and modifications to this description may be made without departing from the spirit or scope of the present disclosure, as defined in the following claims.

1. A compound of Formula (I): 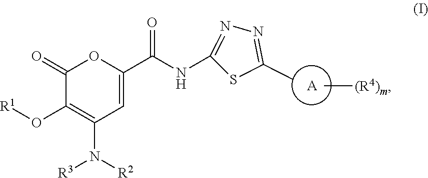
or a pharmaceutically acceptable salt thereof, wherein:
Ring A is a 5-membered monocyclic heteroaryl;
R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄haloalkyl, wherein the alkyl and haloalkyl are independently substituted with 0, 1, 2, 3, or 4 R^{1D}; each R^{1C} is independently hydrogen, C₁-C₃ alkyl, C₁-C₃ haloalkyl, or —OR^{1F}; each R^{1D} is independently halogen, —OR^{1F}, or —N(R^{1F})₂; and each R^{1F} is independently hydrogen, C₁-C₃ alkyl, or C₁-C₃ haloalkyl;
L₃ of group R¹ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O;
R² is hydrogen or C₁-C₆ alkyl substituted with 0, 1, 2, 3, or 4 R^{2A}, and each R^{2A} is independently halogen, —OR^{2B}, or —N(R^{2B})₂, wherein each R^{2B} is independently hydrogen, C₁-C₃ alkyl, or C₁-C₃ haloalkyl;
or R¹ and R² are joined, with the atoms to which they are attached, to form a 6- or 7-membered heterocyclyl independently substituted with 0, 1, 2, 3, or 4 R^{1A};
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, —CN, C₁-C₃ alkyl, or C₁-C₃ haloalkyl;
each R^{3E} is independently hydrogen, C₁-C₄ alkyl, or C₁-C₄ haloalkyl;
each R⁴ is independently halogen, —CN, -L₂-OR^{4A}, -L₂-N(R^{4B})₂, C₁-C₆ alkyl, or C₁-C₆ haloalkyl, wherein each R^{4A} and R^{4B} are independently hydrogen, C₁-C₃ alkyl, C₁-C₃ haloalkyl, or —C(═O)R^{4C}, wherein R^{4C} is C₁-C₆ alkyl or C₁-C₆ haloalkyl;
each L₁ and L₂ is independently a bond, C₁-C₃ alkylene, or C₁-C₃ haloalkylene; and
m is 0, 1 or 2.

2. The compound of claim 1, or a pharmaceutically acceptable salt thereof, wherein:
Ring A is a 5-membered monocyclic heteroaryl;
R¹ is C₁-C₆ alkyl, C₂-C₆ alkenyl, or C₂-C₆ alkynyl, wherein the alkyl, alkenyl, and alkynyl are independently substituted with 0, 1, 2, 3, or 4 R^{1A}; each R^{1A} is independently halogen, —OR^{1B} or —N(R^{1B})₂; and each R^{1B} is independently hydrogen, C₁-C₃ alkyl or C₁-C₃ haloalkyl;
R² is hydrogen or C₁-C₆ alkyl substituted with 0, 1, 2, 3, or 4 R^{2A}, and each R^{2A} is independently halogen, —OR^{2B}, or —N(R^{2B})₂, wherein each R^{2B} is independently hydrogen, C₁-C₃ alkyl, or C₁-C₃ haloalkyl;
R³ is C₁-C₁₀ alkyl, C₂-C₁₀ alkenyl, C₂-C₁₀ alkynyl, C₃-C₁₀ carbocyclyl, or 4- to 10-membered heterocyclyl, wherein the alkyl, alkenyl, alkynyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, C₂-C₃ alkenyl, C₂-C₃ alkynyl, halogen, ═O, -L₁-CN, -L₁-SOR^{3C}, -L₁-SO₂R^{3C}, -L₁-SR^{3B}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, C₁-C₃ alkyl, or C₁-C₃ haloalkyl;
each R^{3E} is independently hydrogen, C₁-C₃ alkyl, or C₁-C₃ haloalkyl;
each R⁴ is independently halogen, —CN, -L₂-OR^{4A}, -L₂-N(R^{4B})₂, C₁-C₆ alkyl, or C₁-C₆ haloalkyl, wherein each R^{4A} and R^{4B} are independently hydrogen, C₁-C₃ alkyl, or C₁-C₃ haloalkyl;
each L₁ and L₂ is independently a bond, C₁-C₃ alkylene, or C₁-C₃ haloalkylene; and
m is 0, 1 or 2.

3. (canceled)

4. The compound of claim 2, wherein the amino moiety 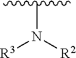
is a group of formula (i-a): 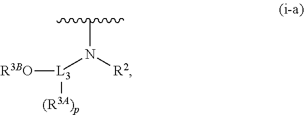
wherein L₃ of formula (i-a) is C₁-C₁₀ alkylene, C₂-C₁₀ alkenylene, or C₂-C₁₀ alkynylene and p is 0, 1, 2, or 3; and the compound is of Formula (I′): 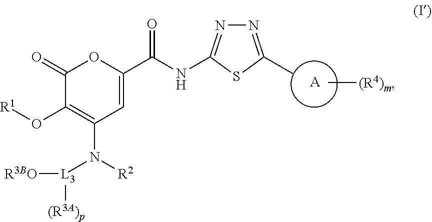
or a pharmaceutically acceptable salt thereof.

5. The compound of claim 2, wherein the amino moiety 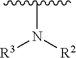
is a group of formula (ii-a): 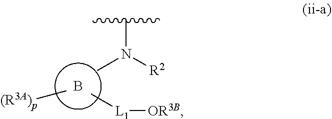
wherein Ring B of formula (ii-a) is a C₃-C₁₀ carbocyclyl or 4- to 10-membered heterocyclyl and p is 0, 1, 2, or 3; and the compound is of Formula (I″): 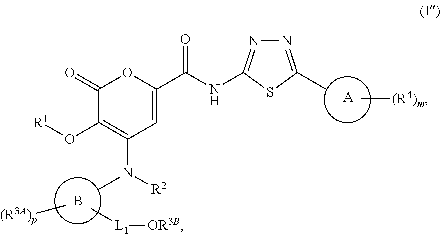
or a pharmaceutically acceptable salt thereof.

6. The compound of claim 2, wherein the amino moiety 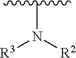
is a group of formula (iii-a): 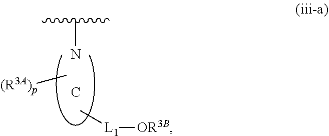
wherein Ring C of formula (iii-a) is a 5- to 10-membered heterocyclyl and p is 0, 1, 2, or 3; and the compound is of Formula (I′″): 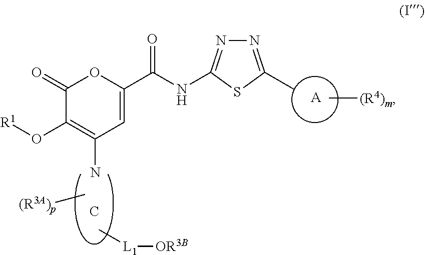
or a pharmaceutically acceptable salt thereof.

7. The compound of claim 2, wherein the compound is of Formula (I″″): 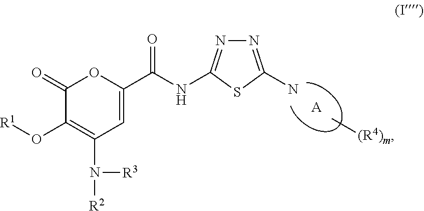
or a pharmaceutically acceptable salt thereof, wherein the nitrogen atom of the heteroaryl Ring A is directly linked to the thiadiazole moiety.

8. The compound of claim 2, or a pharmaceutically acceptable salt thereof, wherein R¹ is C₁-C₆ alkyl substituted with 0, 1, 2, 3, or 4 R^{1A}.

9. The compound of claim 8, or a pharmaceutically acceptable salt thereof, wherein R¹ is —CH₃, —CH₂—C(CH₃)₂—CH₂OCH₃, —CH₂CH₂OH, or —CH₂CH₂OCH₃.

10. The compound of claim 2, or a pharmaceutically acceptable salt thereof, wherein R² is hydrogen.

11-15. (canceled)

16. The compound of claim 2, or a pharmaceutically acceptable salt thereof, wherein 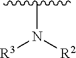
is selected from the group consisting of:

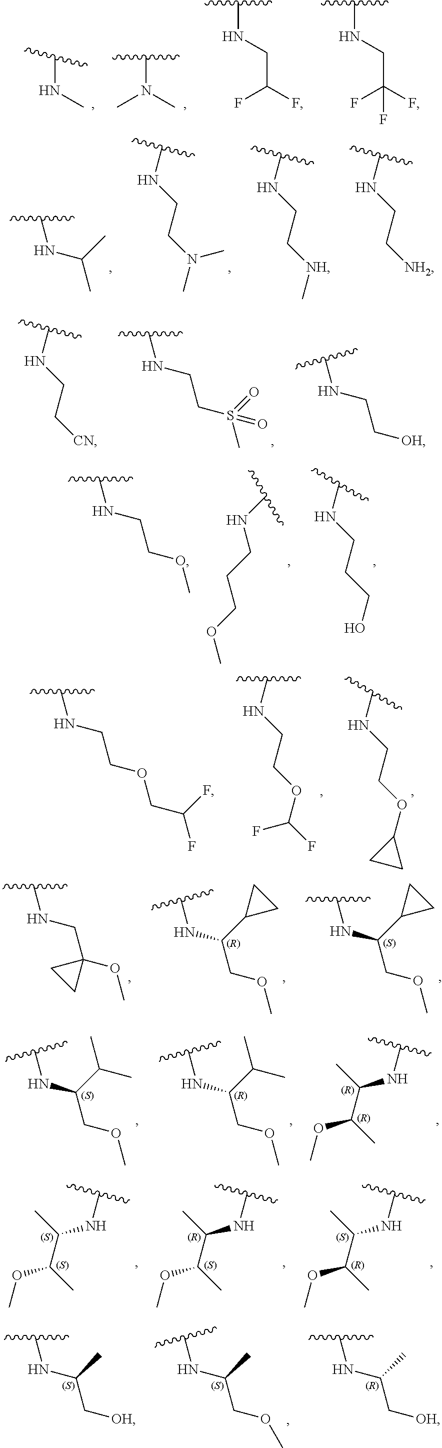

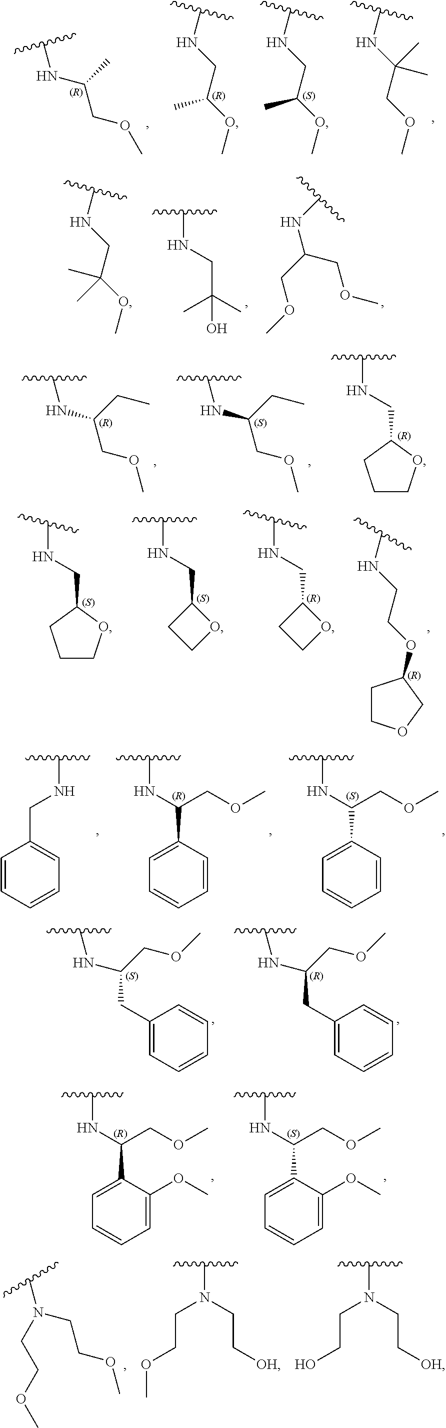

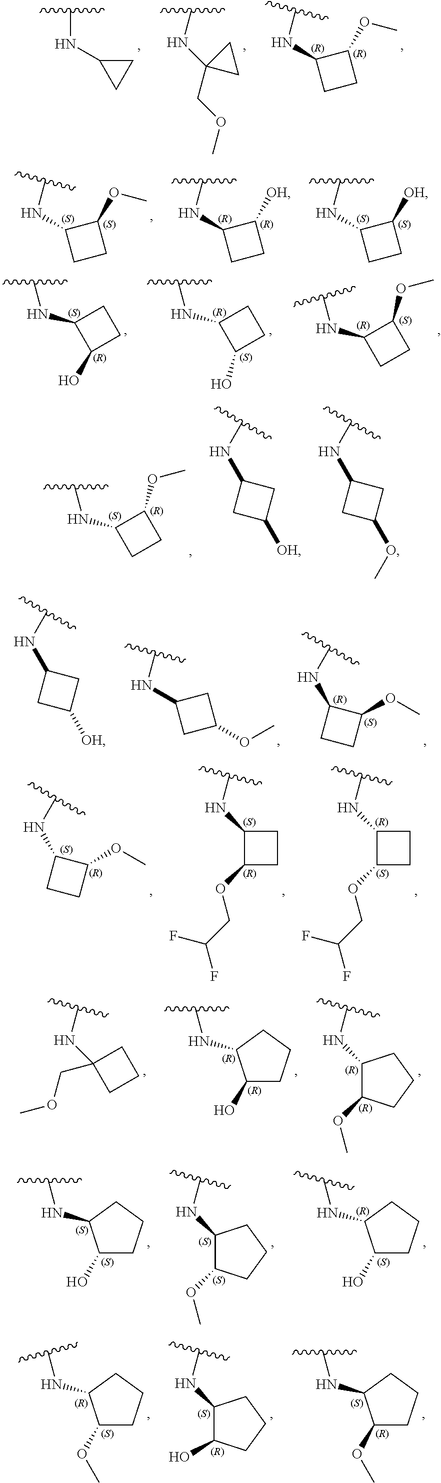

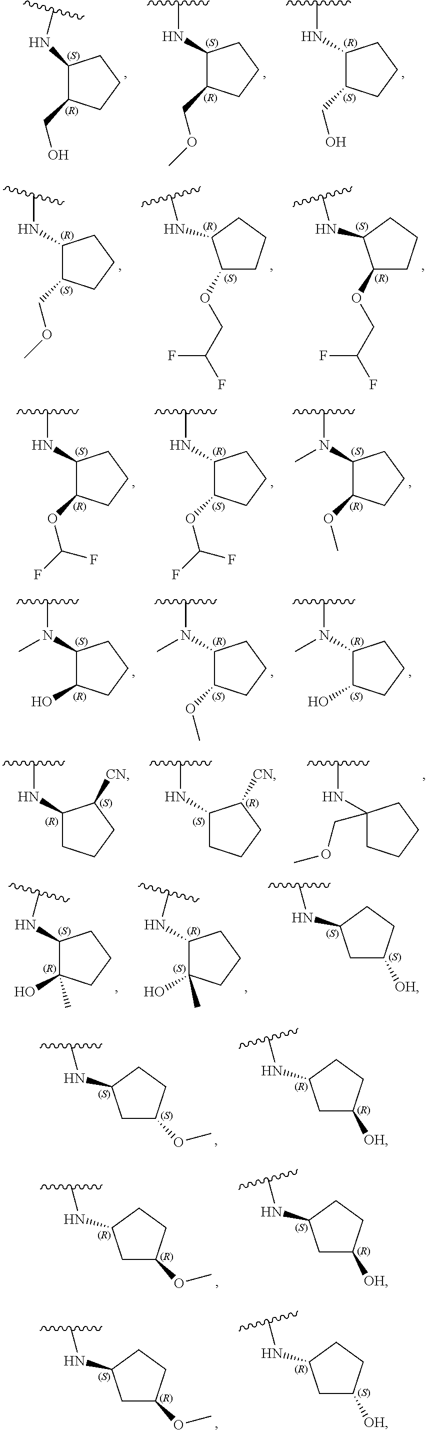

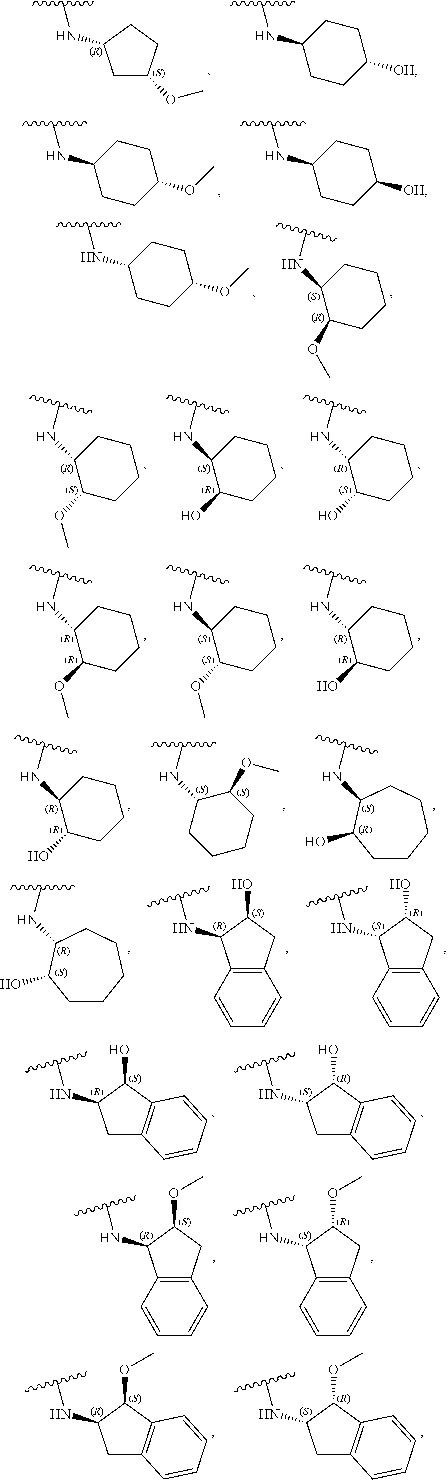

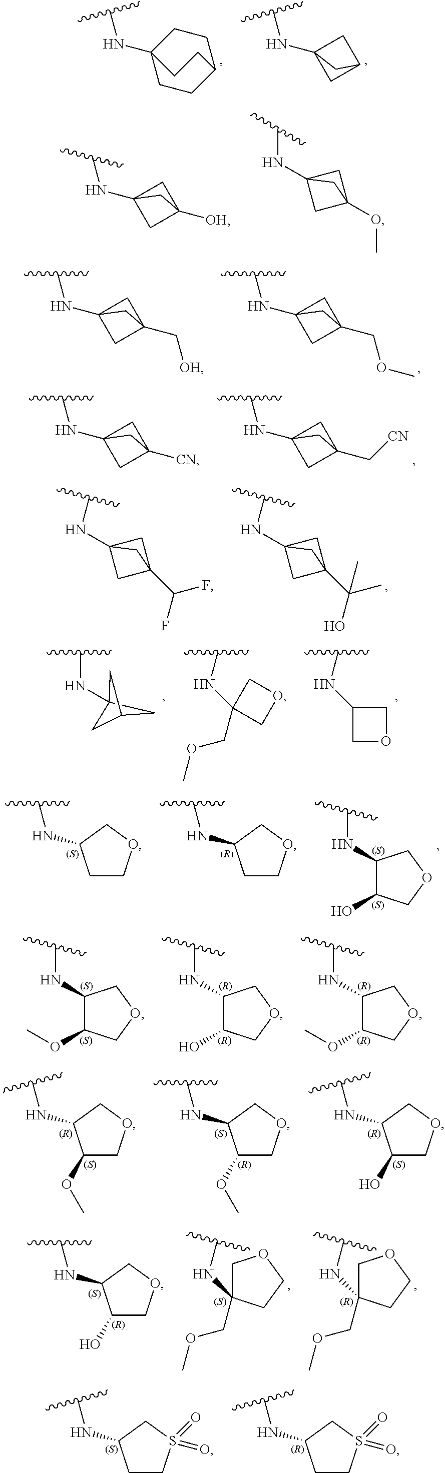

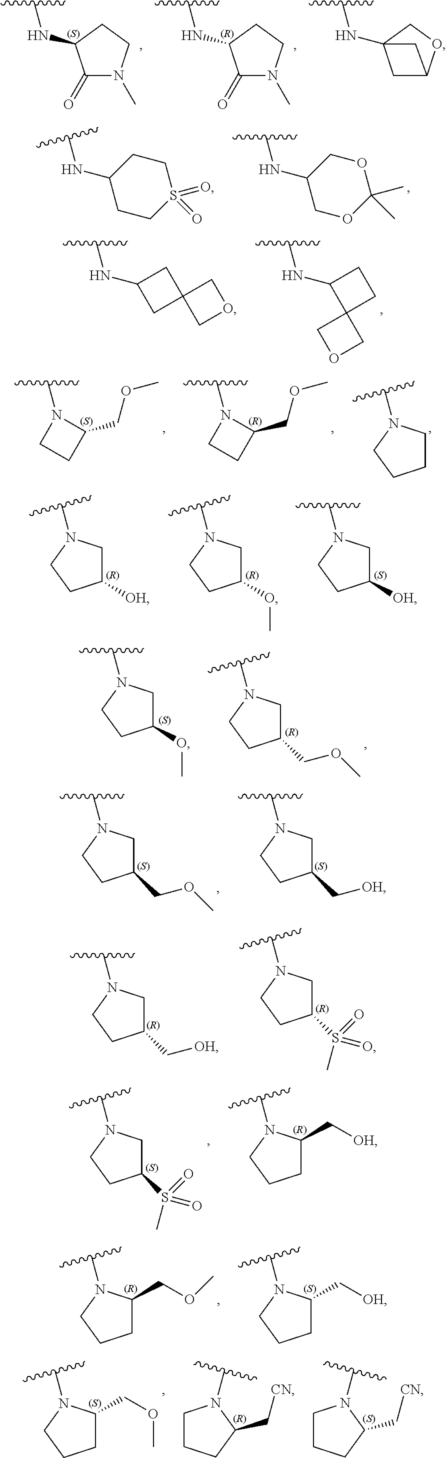

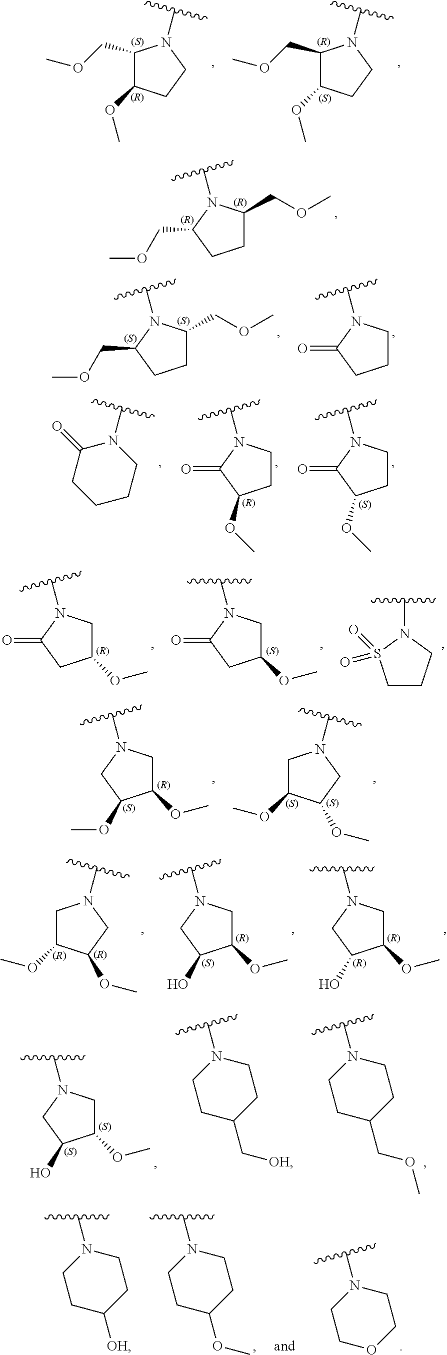

17-19. (canceled)

20. The compound of claim 2, or pharmaceutically acceptable salt thereof, wherein Ring A is:

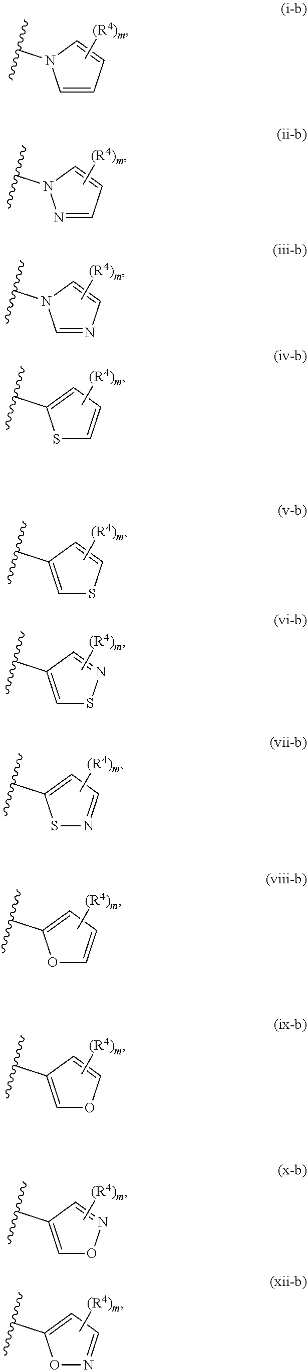

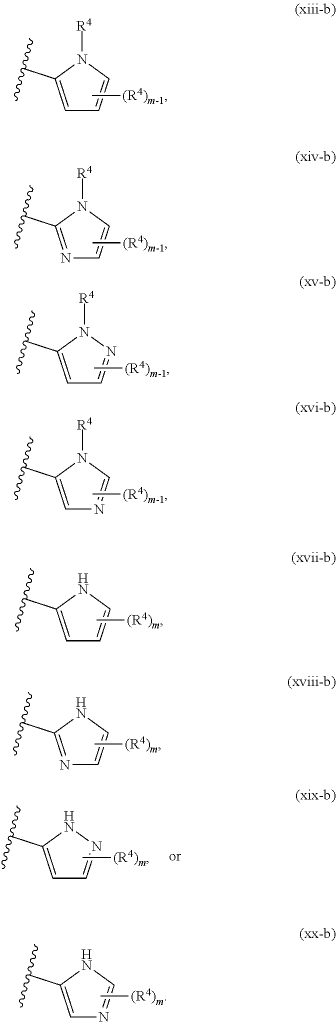

21-22. (canceled)

23. The compound of claim 20, or a pharmaceutically acceptable salt thereof, wherein each R⁴ is independently —CH₃, —CH₂CH₃, —CHF₂, —CF₃, —Cl, —CN, —NH₂, or —CH₂OH.

24. The compound of claim 2, or a pharmaceutically acceptable salt thereof, wherein each L₁ and L₂ is independently a bond or C₁-C₃ alkylene.

25. (canceled)

26. The compound of claim 23, or a pharmaceutically acceptable salt thereof, wherein m is 1.

27. (canceled)

28. The compound of claim 20, or a pharmaceutically acceptable salt thereof, wherein Ring A is:

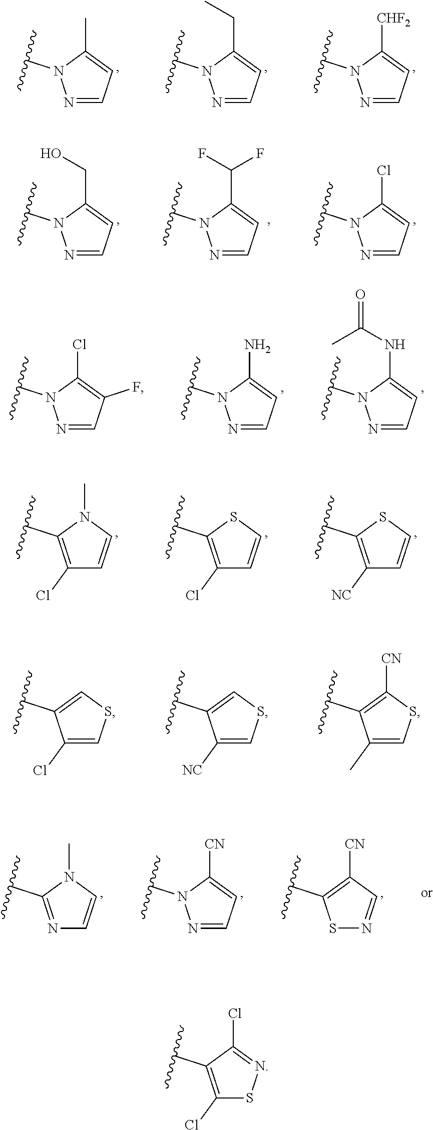

29. (canceled)

30. The compound of claim 1, wherein the compound is of Formula (I-a), (I-b), (I-c), (I-d), (I-e), (I-f), (I-g), (I-a-1), (I-a-2), (I-a-3), (I-f-1), (I-f-2), (I-f-3), (I-BC-a), or (I-BC-b): 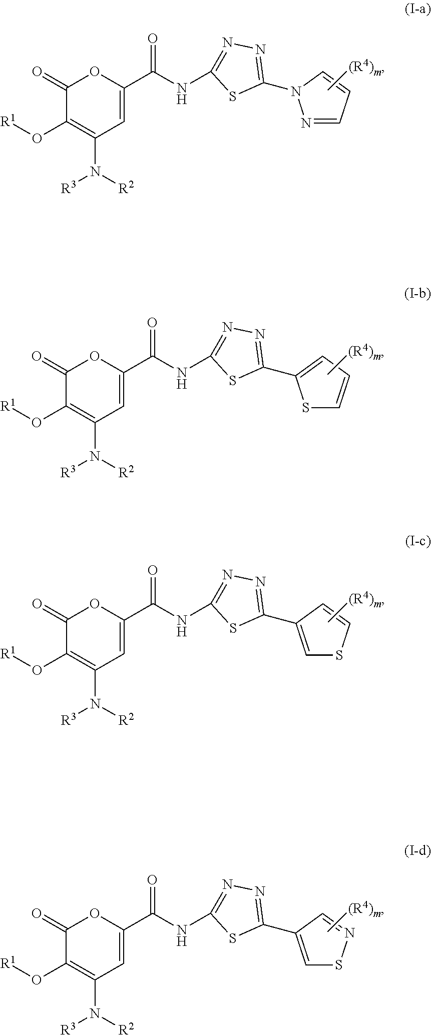 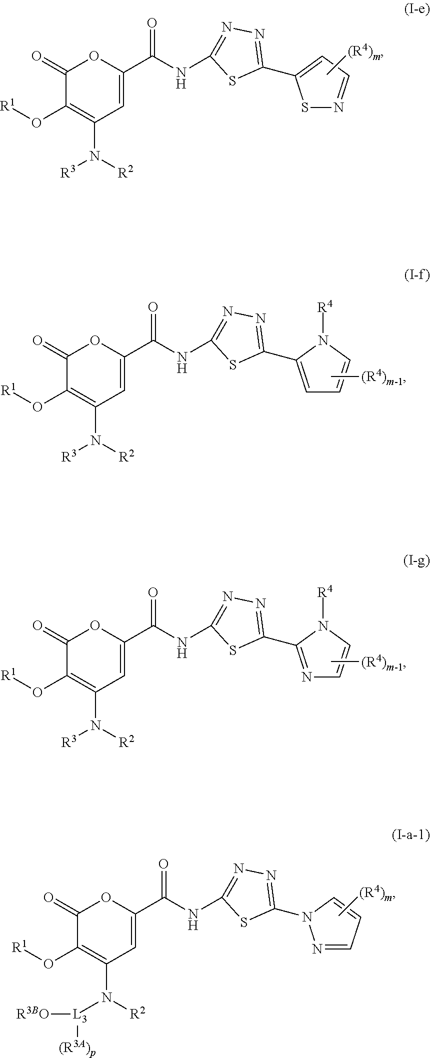 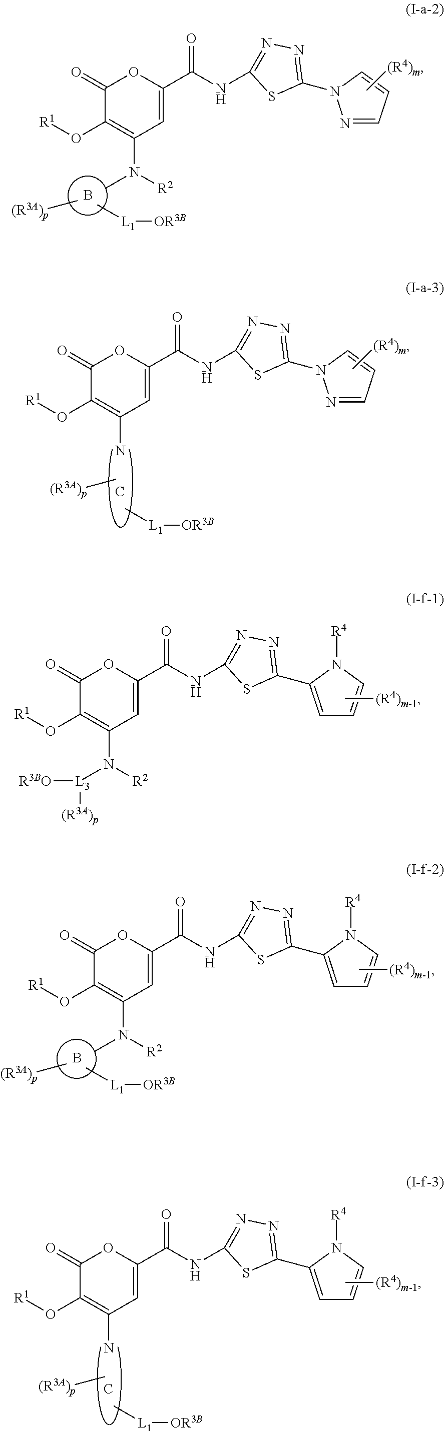 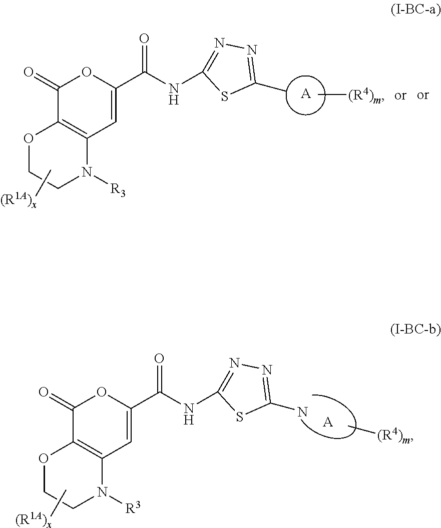
or a pharmaceutically acceptable salt of any of the foregoing wherein x is 0, 1, 2, 3, or 4.

31. The compound of claim 2, or pharmaceutically acceptable salt thereof, wherein:
R¹ is C₁-C₆ alkyl, substituted with 0, 1, 2, 3, or 4 R^{1A}; each R^{1A} is independently-OR^{1B}; and
each R^{1B} is independently hydrogen or C₁-C₃alkyl;
R² is hydrogen or C₁-C₆ alkyl substituted with 0, 1, 2, 3, or 4 R^{2A}, and each R^{2A} is independently —OR^{2B}, wherein each R^{2B} is independently hydrogen or C₁-C₃ alkyl;
R³ is C₁-C₁₀ alkyl, C₃-C₁₀ carbocyclyl, or 4- to 10-membered heterocyclyl, wherein the alkyl, carbocyclyl, and heterocyclyl are independently substituted with 0, 1, 2, 3, or 4 R^{3A};
or R² and R³ are joined, with the atom to which they are attached, to form a 4- to 10-membered heterocyclyl independently substituted with 0, 1, 2, 3, or 4 R^{3A};
each R^{3A} is independently C₁-C₃ alkyl, halogen, ═O, -L₁-CN, -L₁-SO₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, carbocyclyl, heterocyclyl, aryl, and heteroaryl are independently substituted with 0, 1, 2, 3, or 4 R^{3D};
each R^{3C} is independently C₁-C₃ alkyl;
each R^{3D} is independently halogen or —OR^{3E};
R^{3E} is C₁-C₃ alkyl;
each R⁴ is independently halogen, —CN, -L₂-OR^{4A}, -L₂-N(R^{4B})₂, C₁-C₆ alkyl, or C₁-C₆ haloalkyl;
each L₁ and L₂ is independently a bond or C₁-C₃ alkylene; and
m is 1 or 2.

32. The compound of claim 1, wherein the compound is selected from the group consisting of: 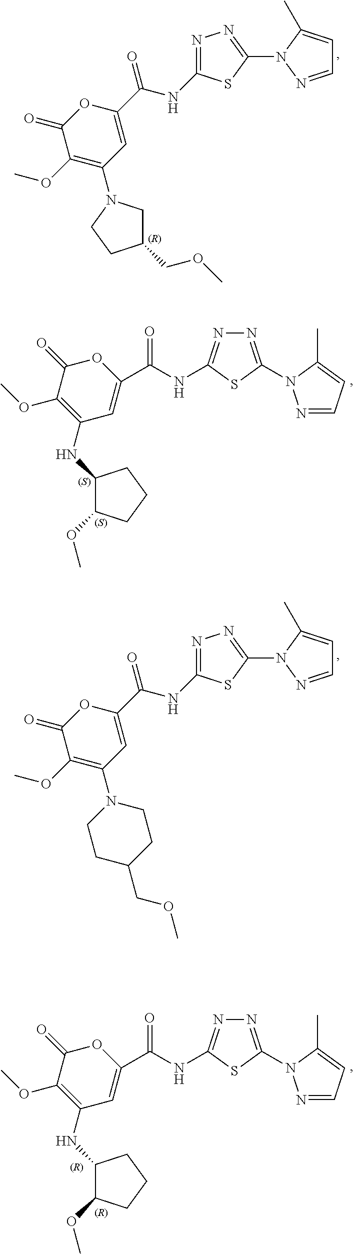 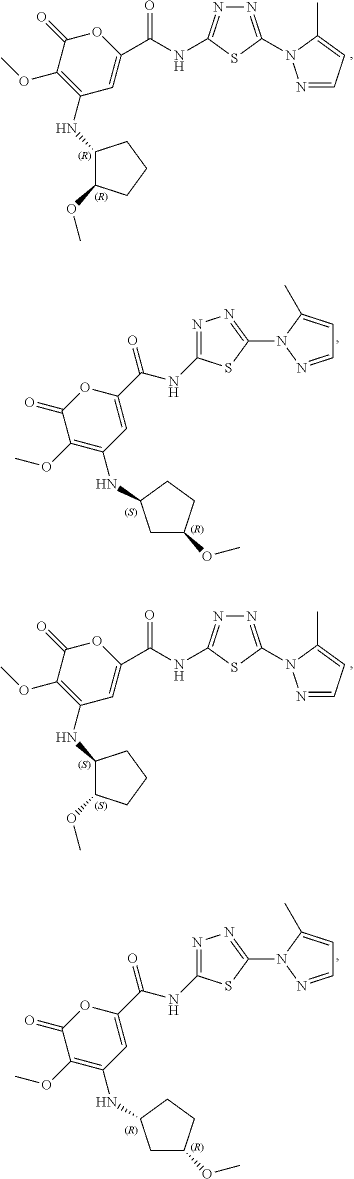 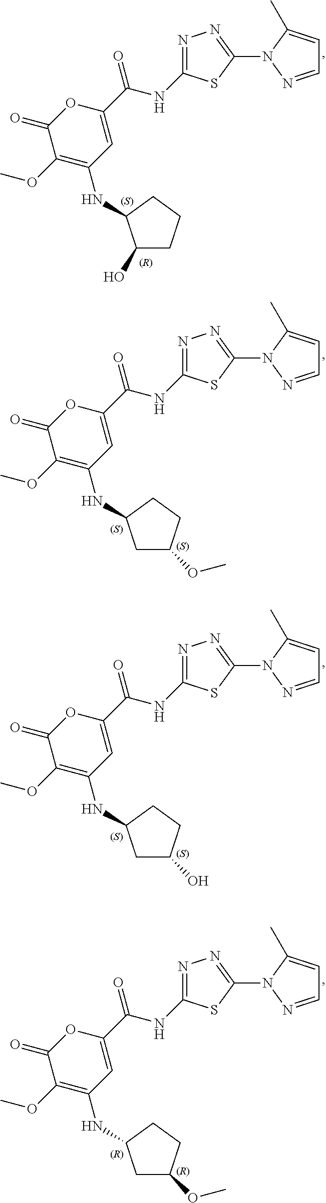 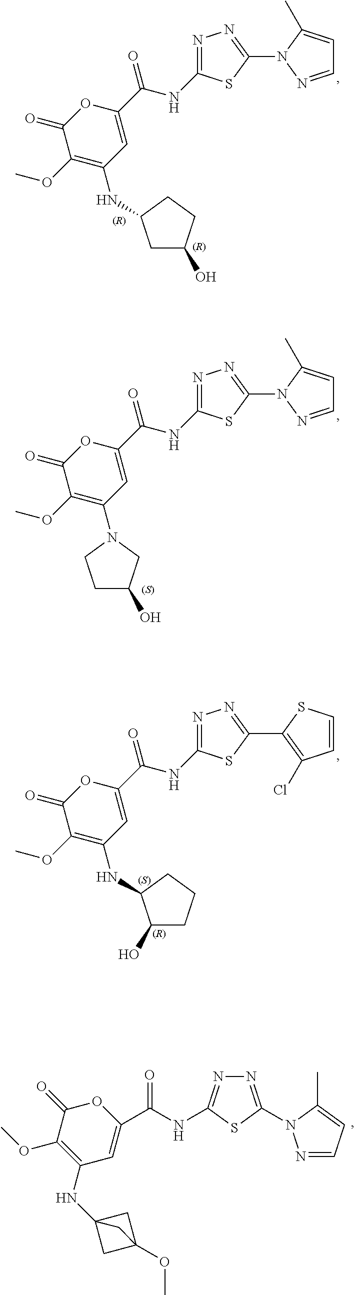 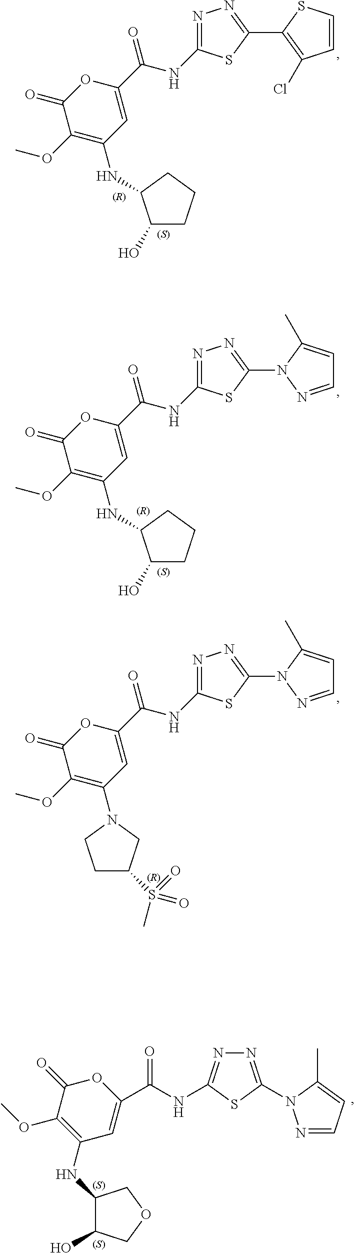 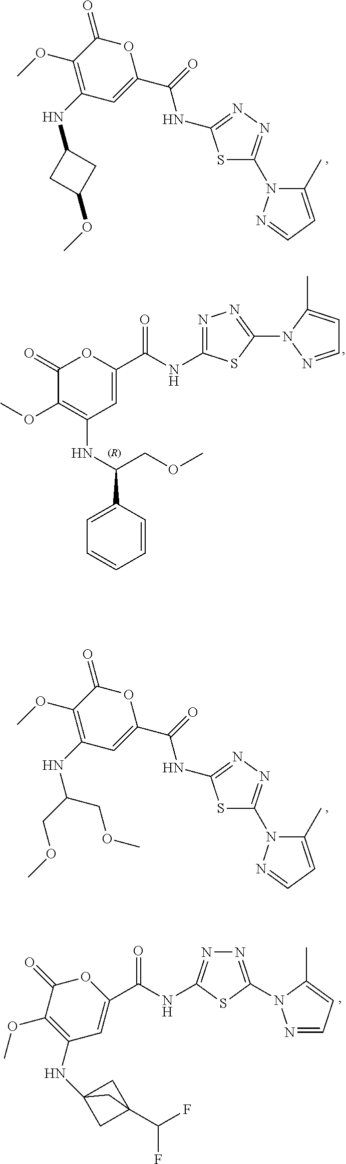 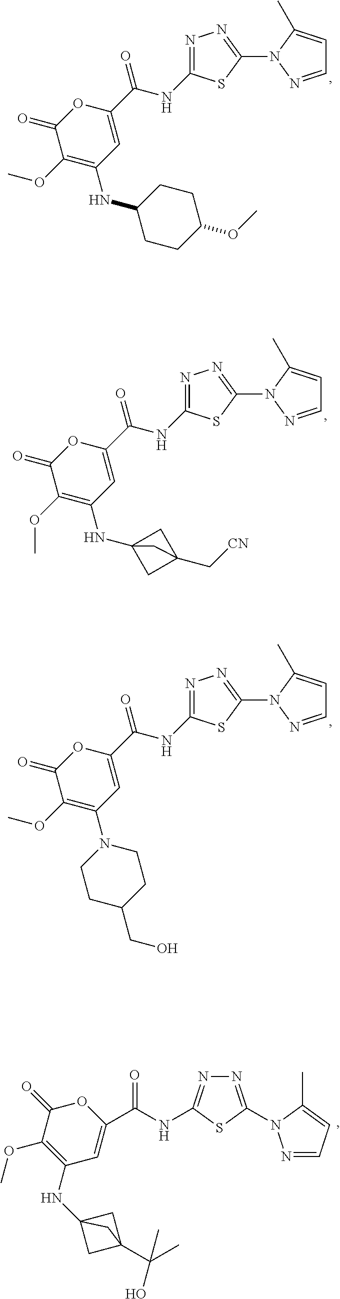 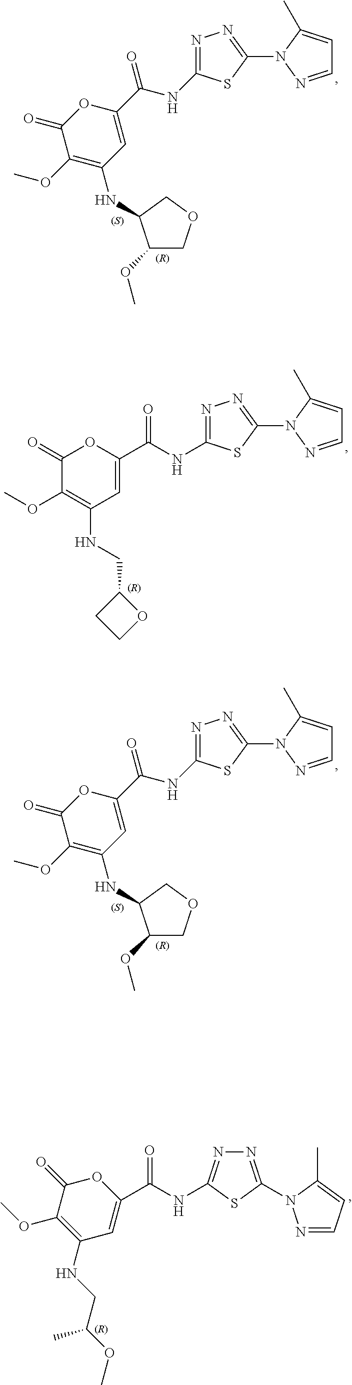 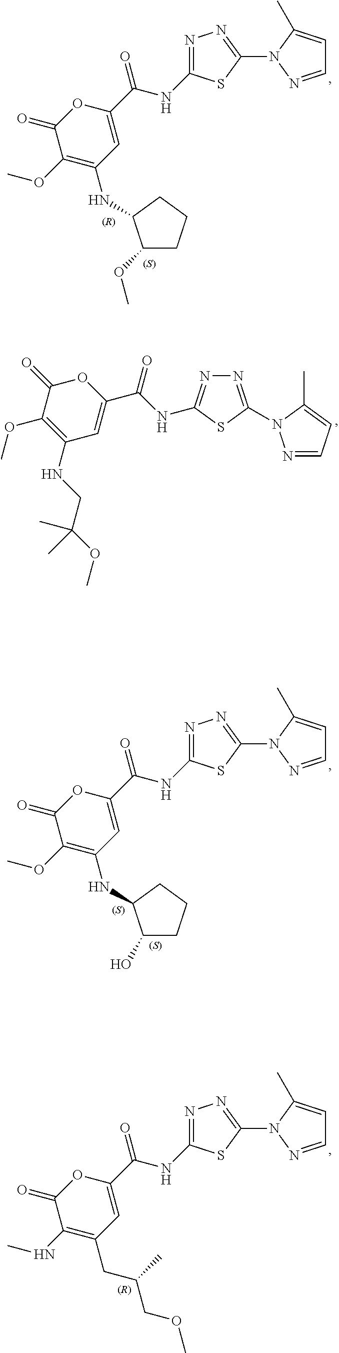 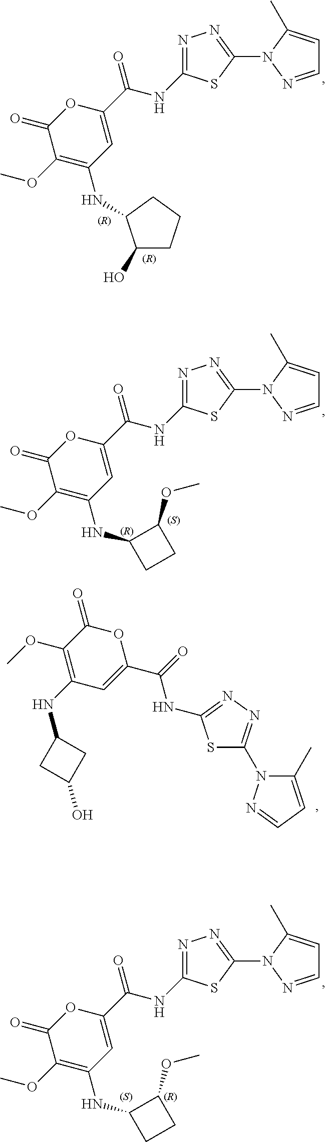 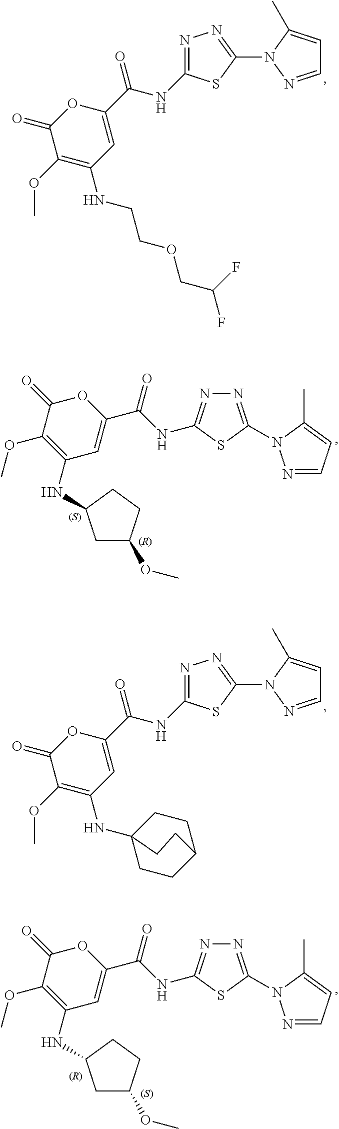 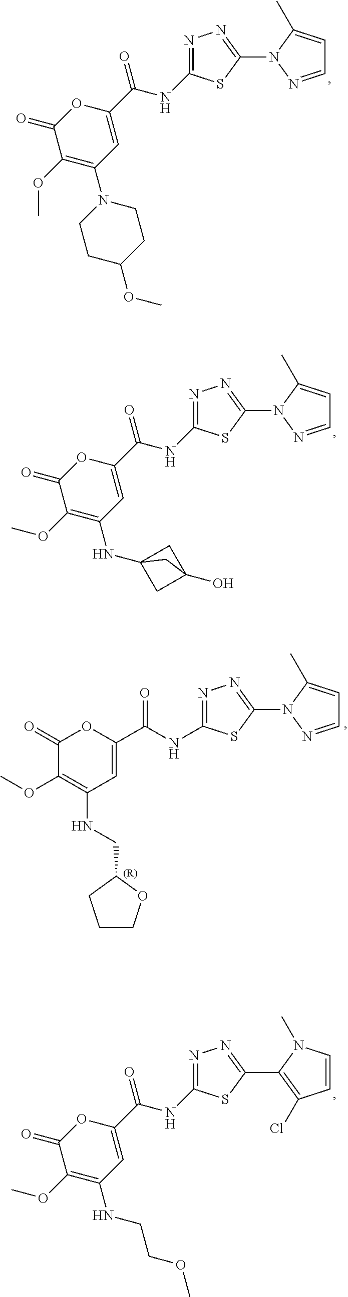 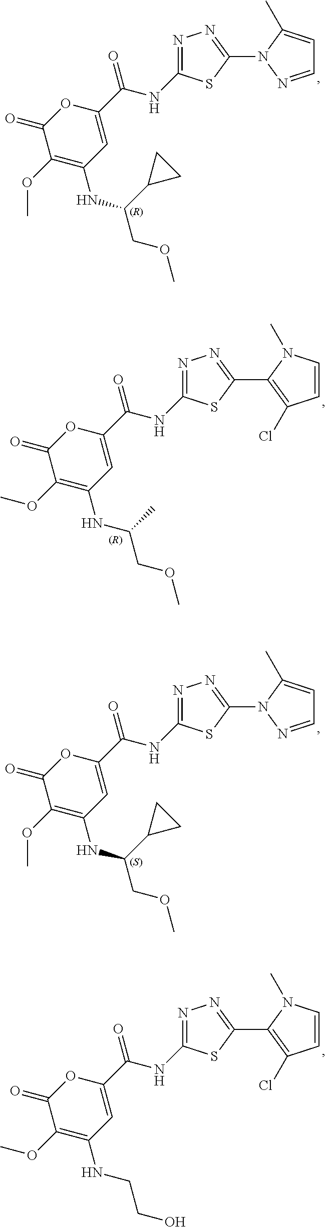 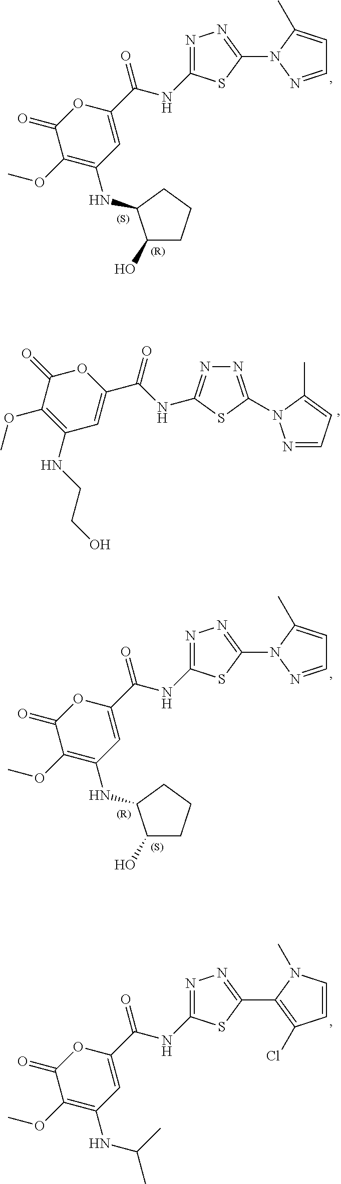 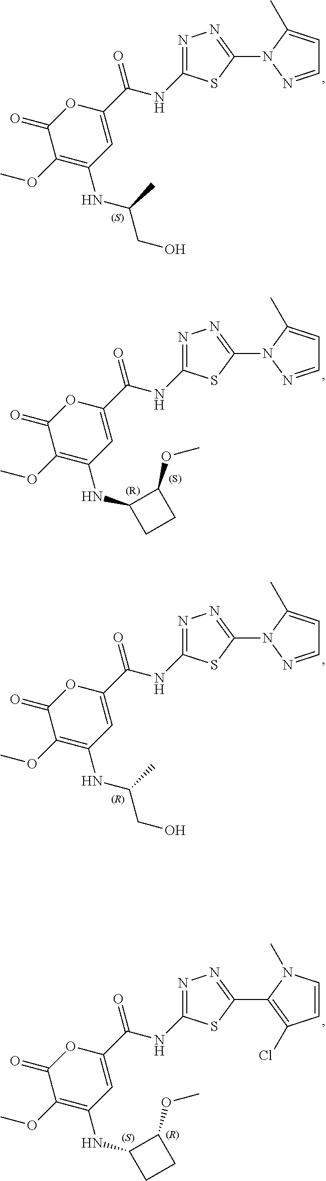 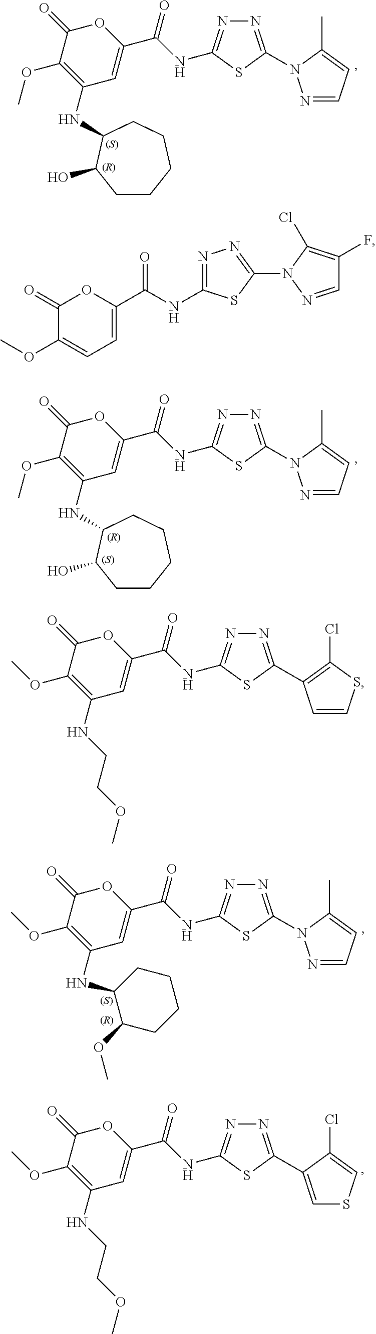 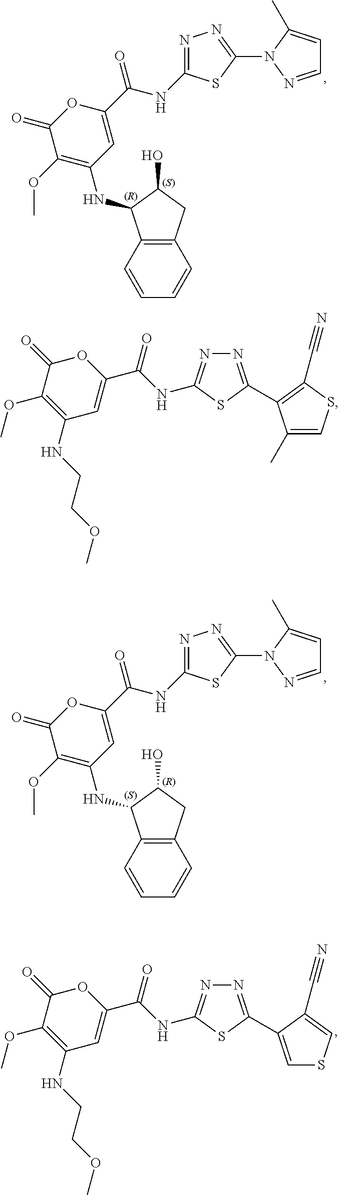 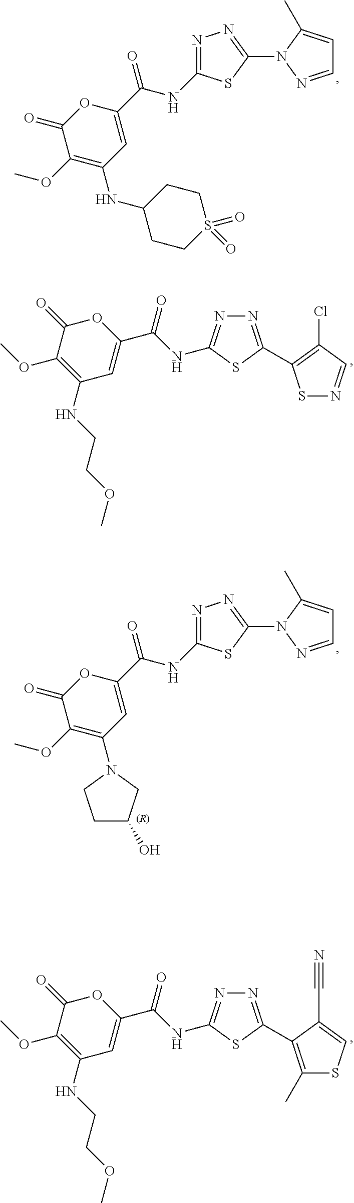 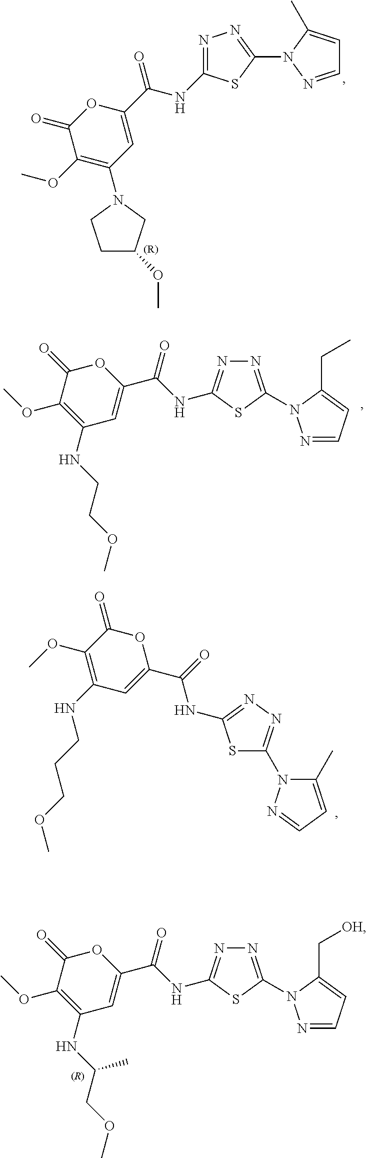 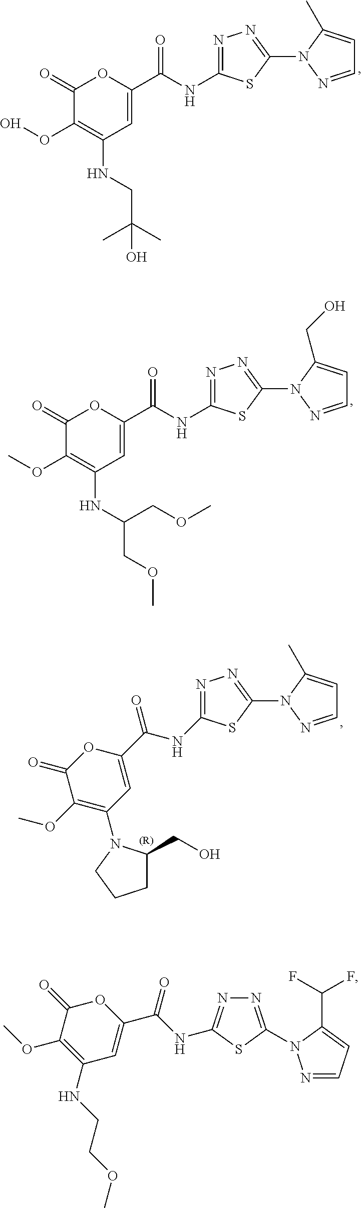 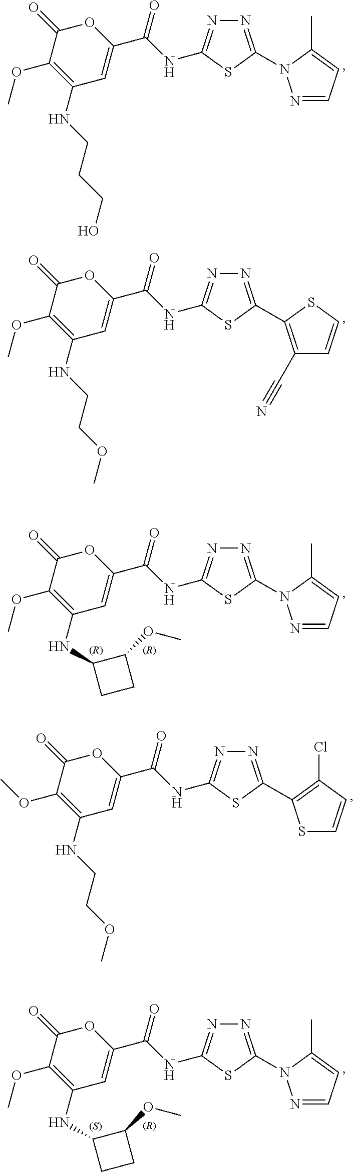 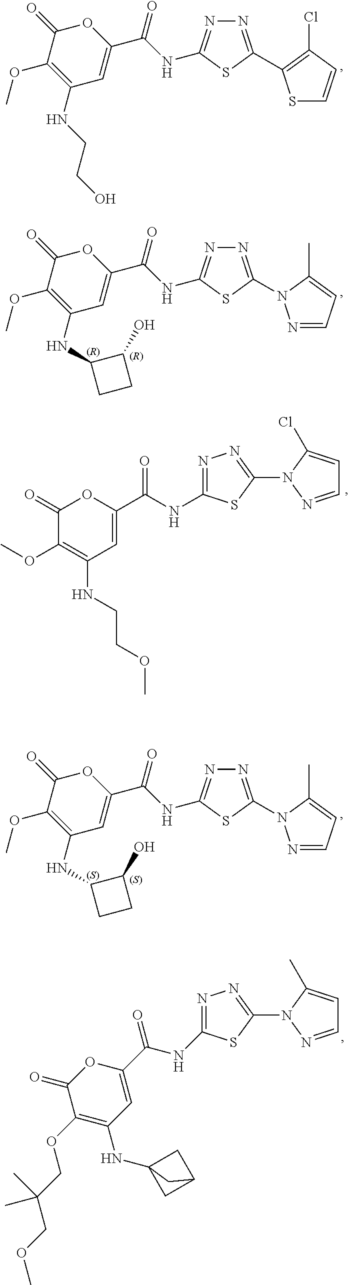 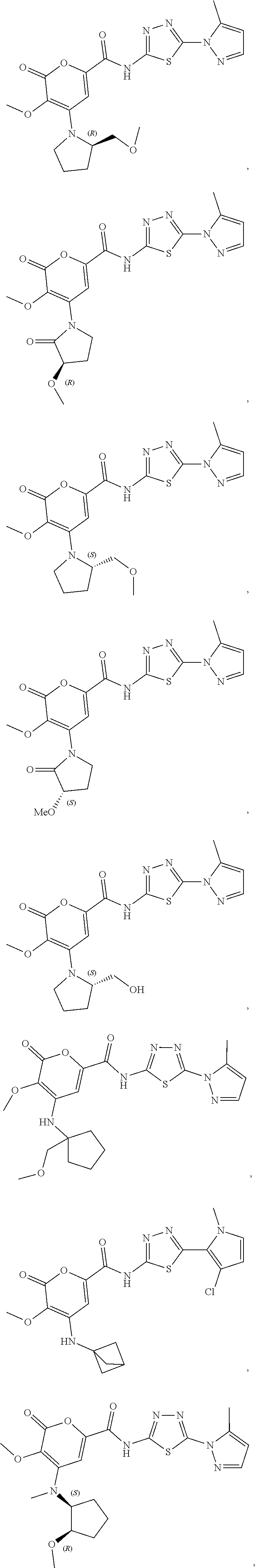 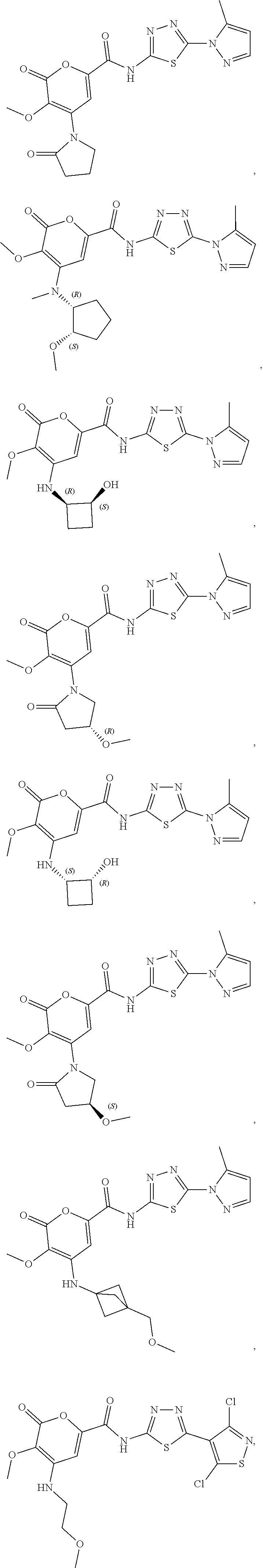 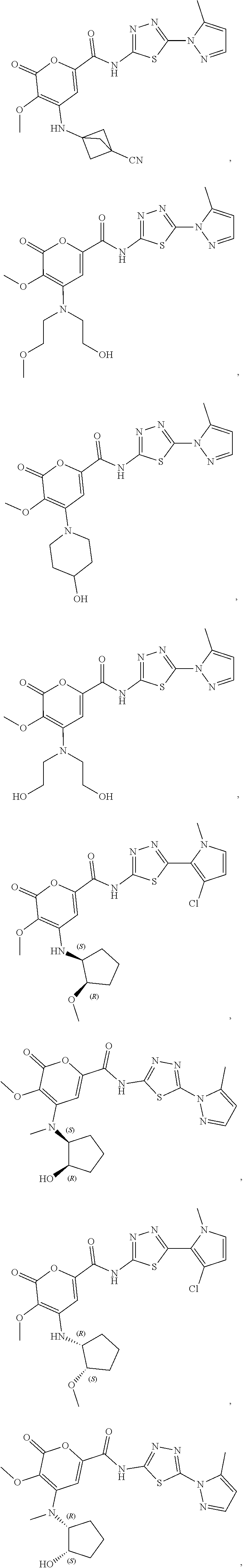 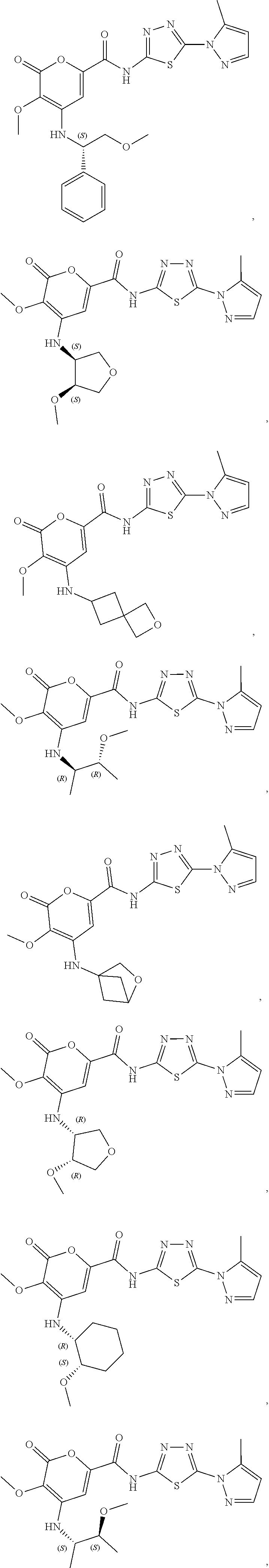 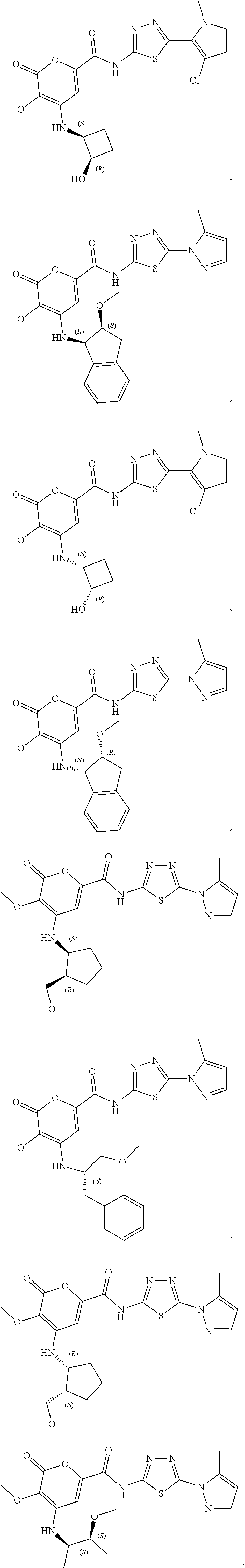 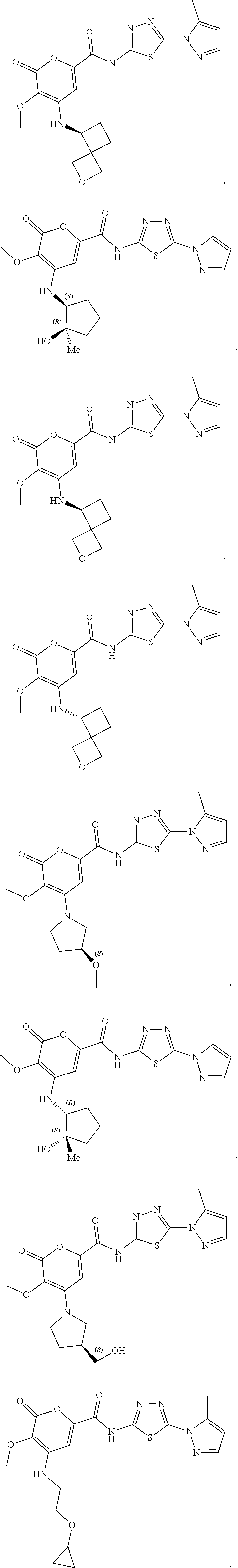 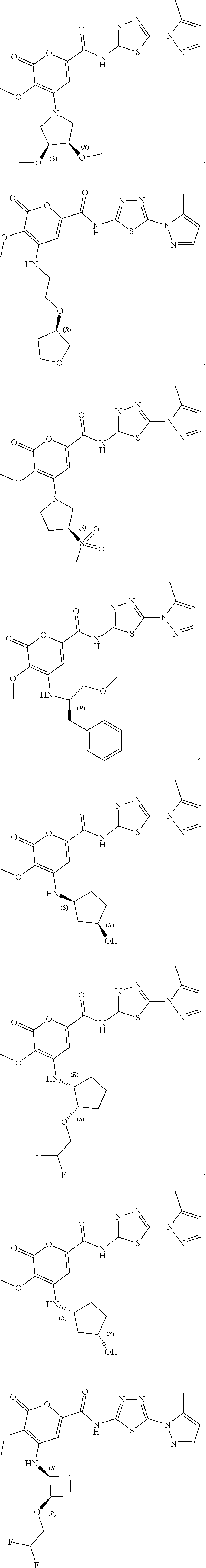 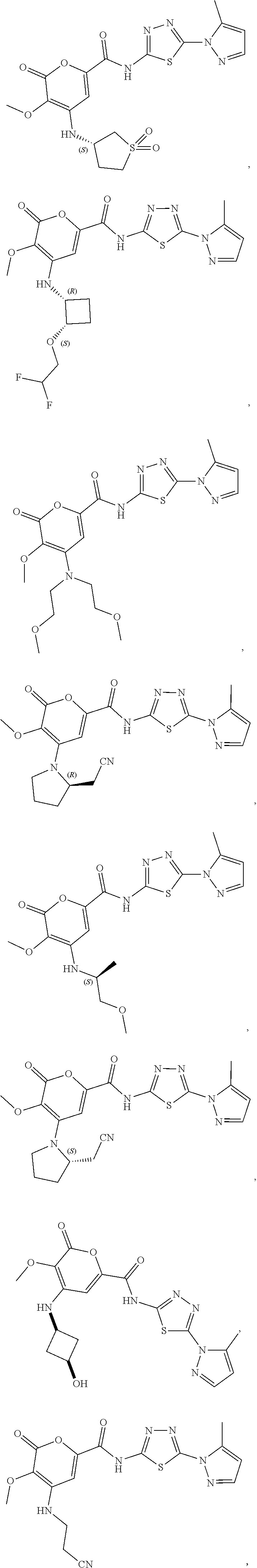 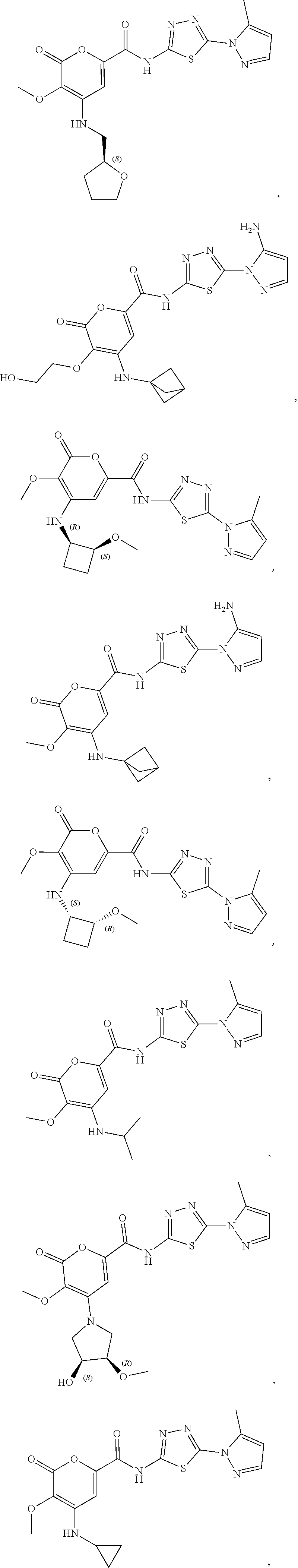 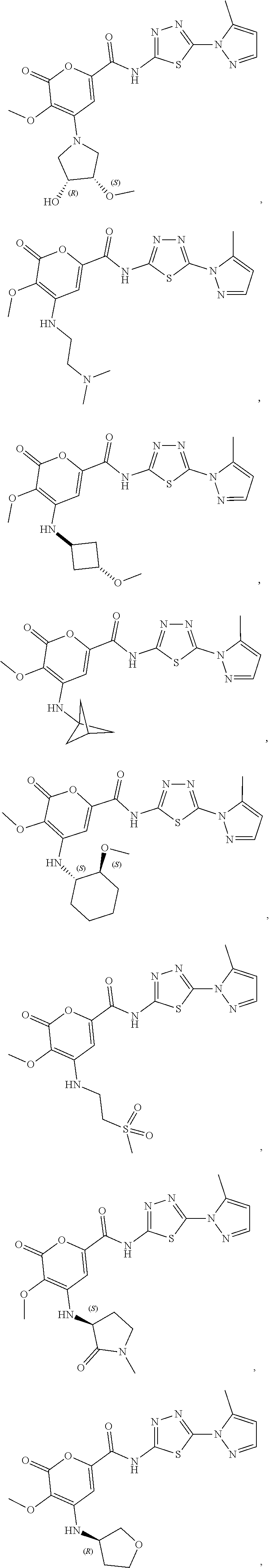 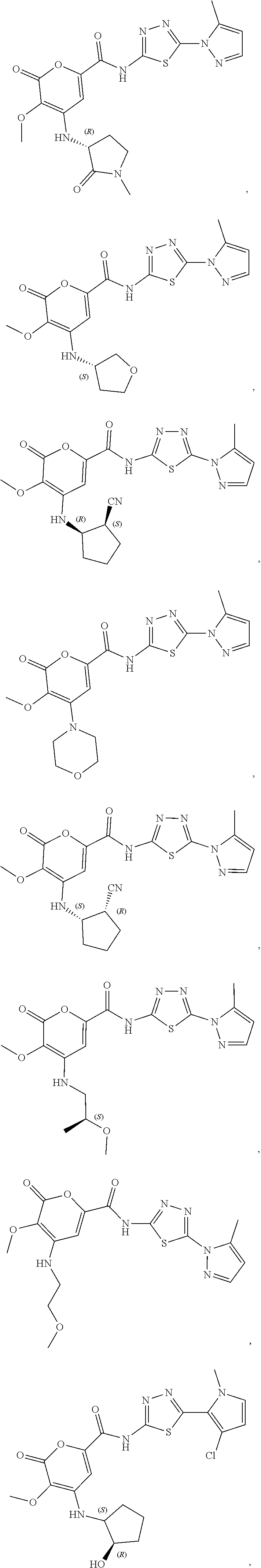 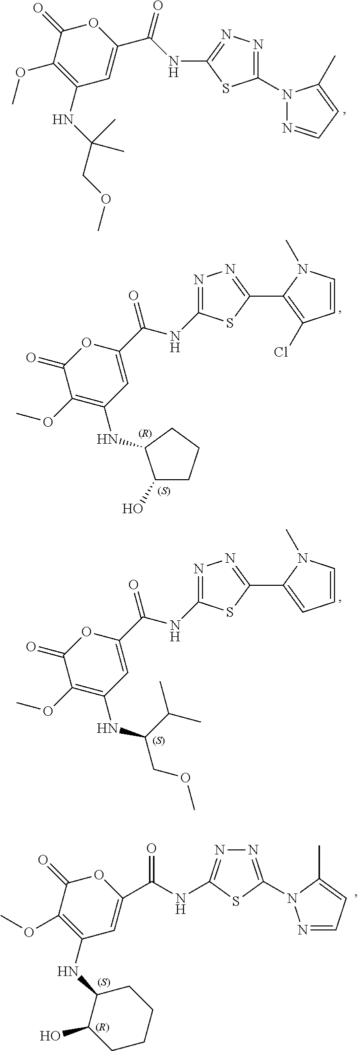 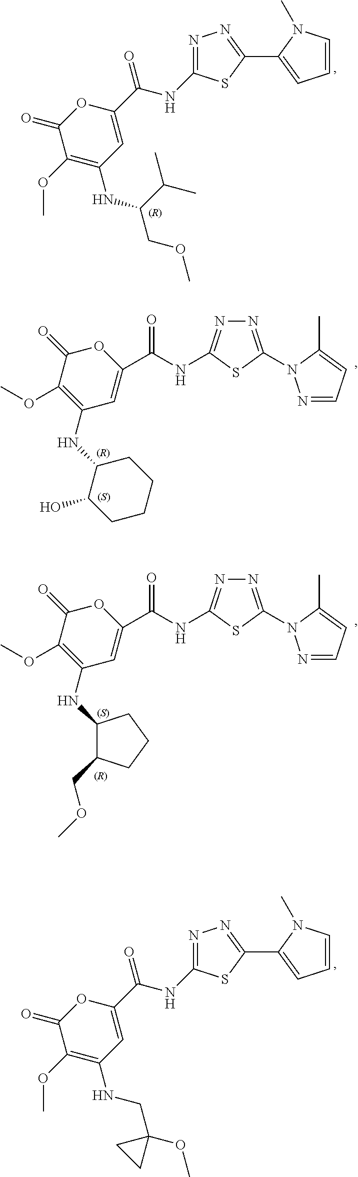 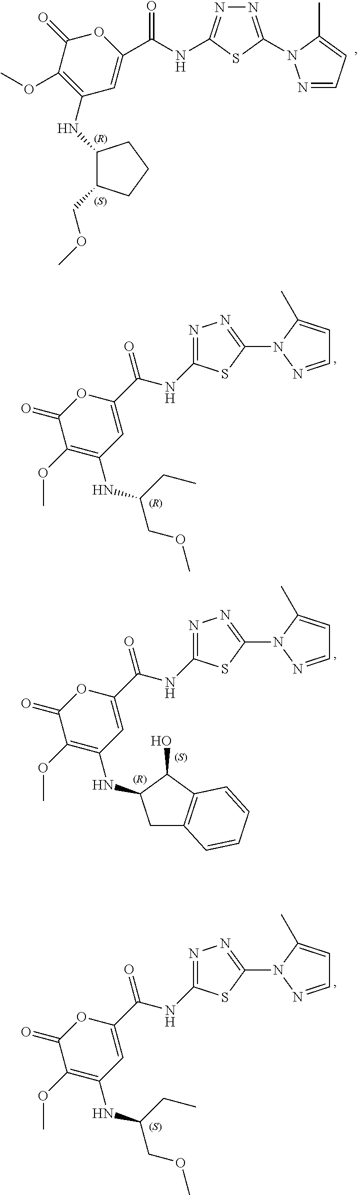 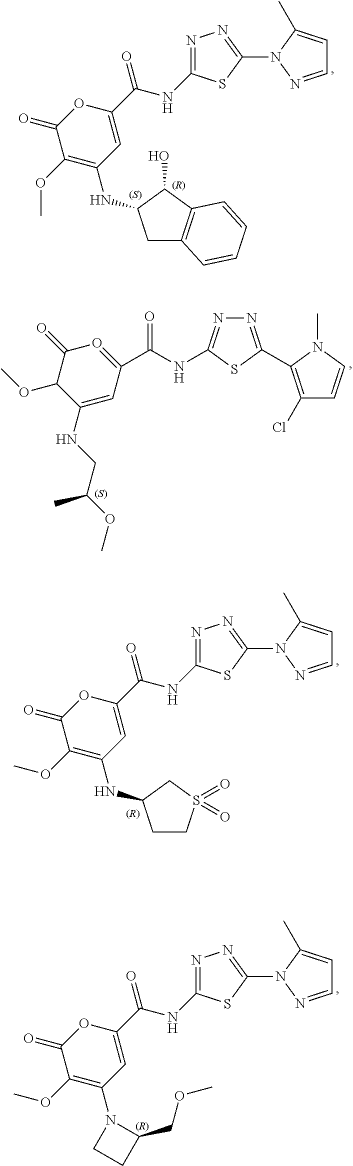 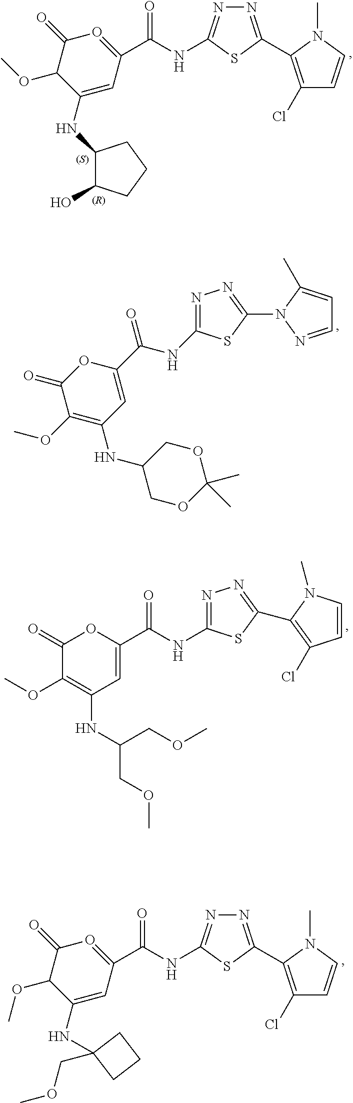 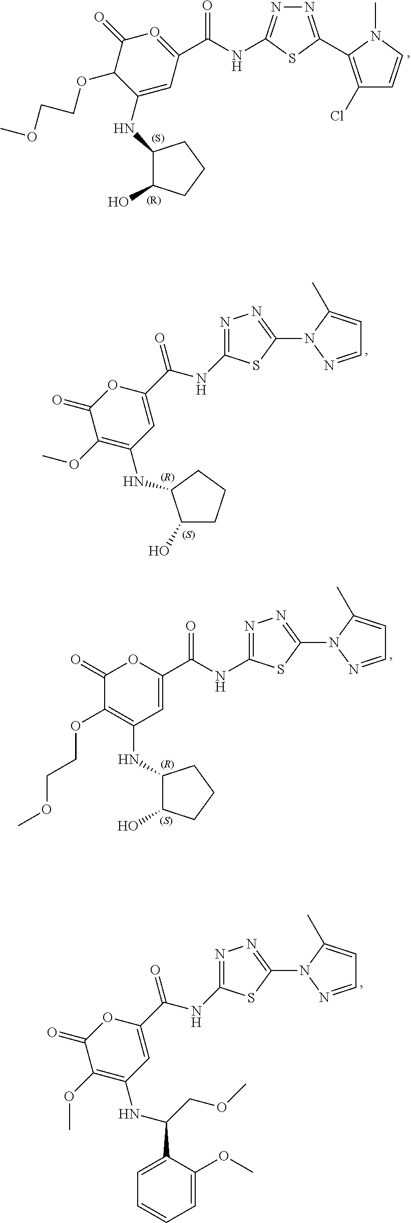 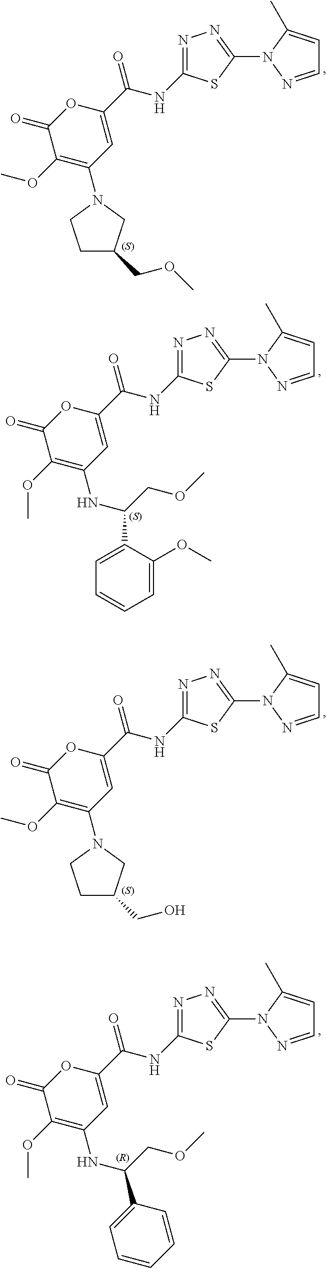 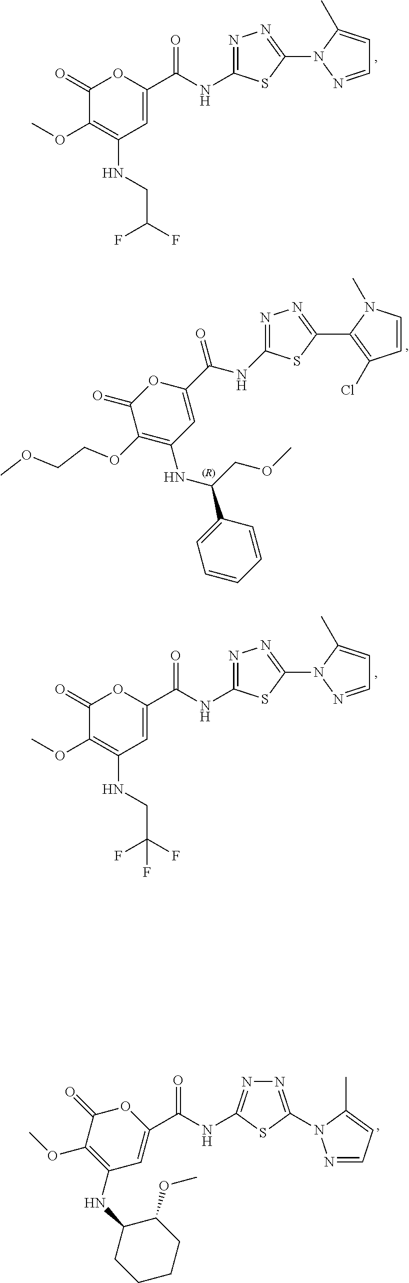 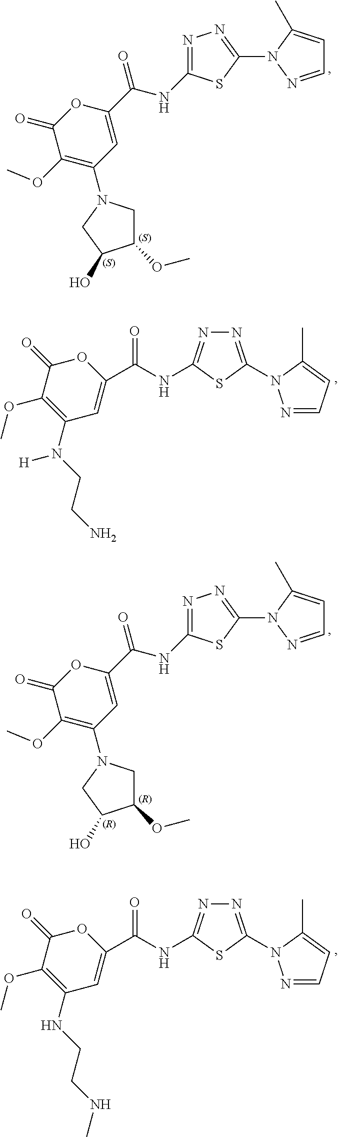 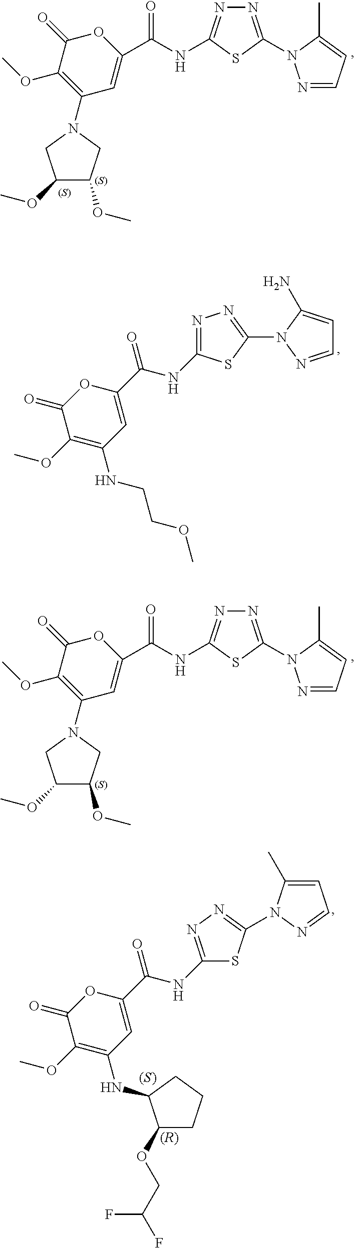 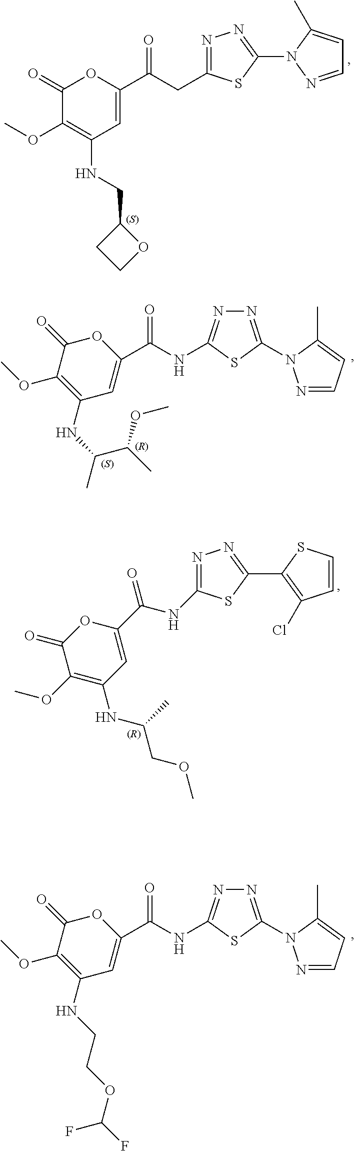 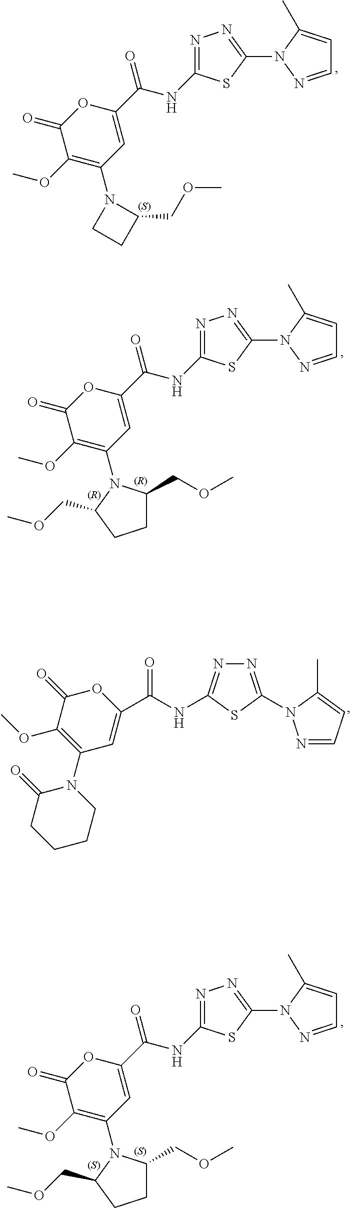 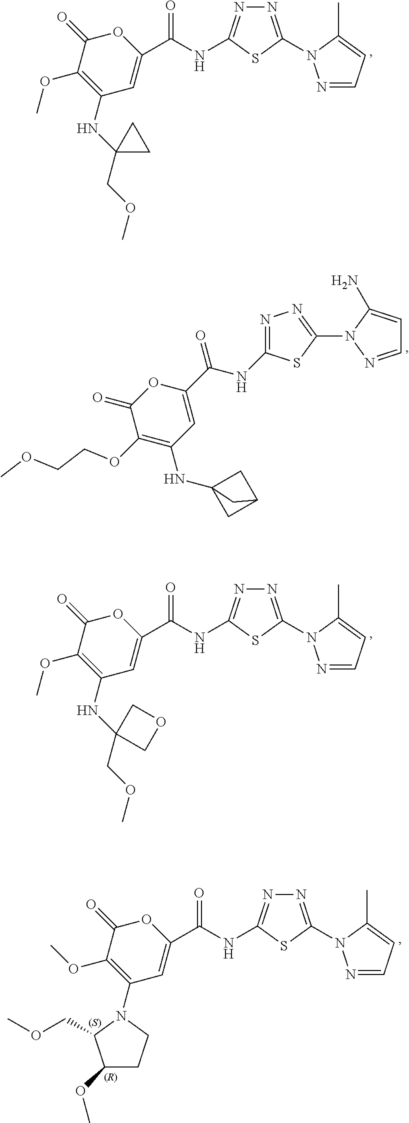 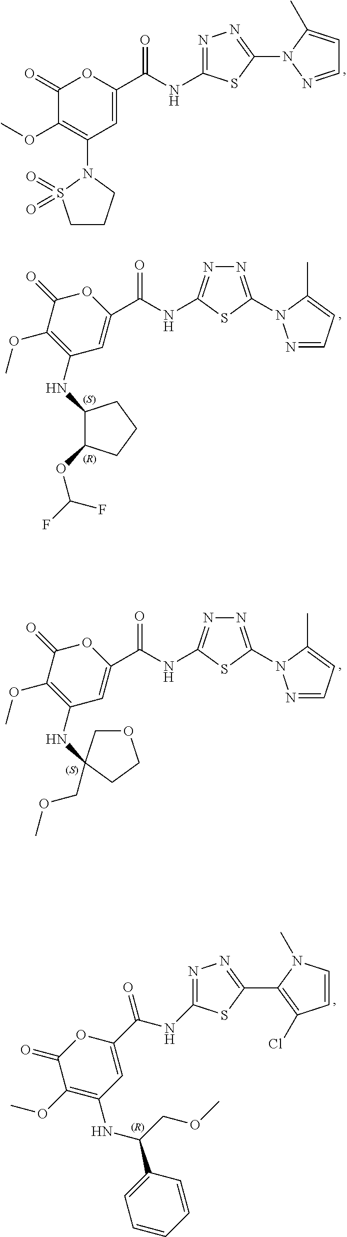 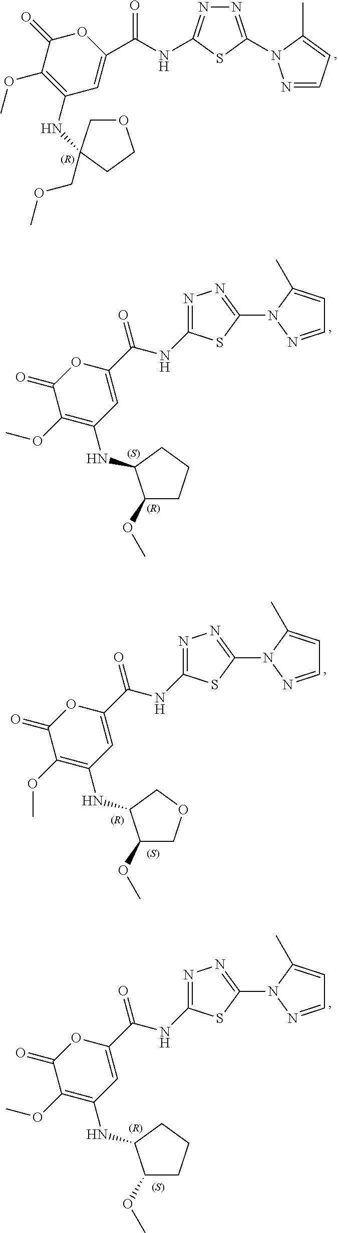 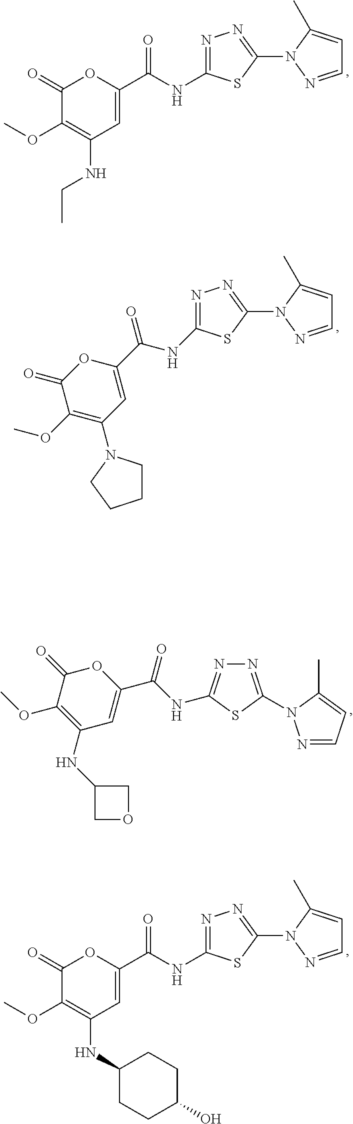 [SMILES: CCNc1cc(C(=O)Nc2nnc(-n3nccc3C)s2)oc(=O)c1OC ; COc1c(N2CCCC2)cc(C(=O)Nc2nnc(-n3nccc3C)s2)oc1=O ; COc1c(NC2COC2)cc(C(=O)Nc2nnc(-n3nccc3C)s2)oc1=O ; COc1c(N[C@H]2CC[C@H](O)CC2)cc(C(=O)Nc2nnc(-n3nccc3C)s2)oc1=O] 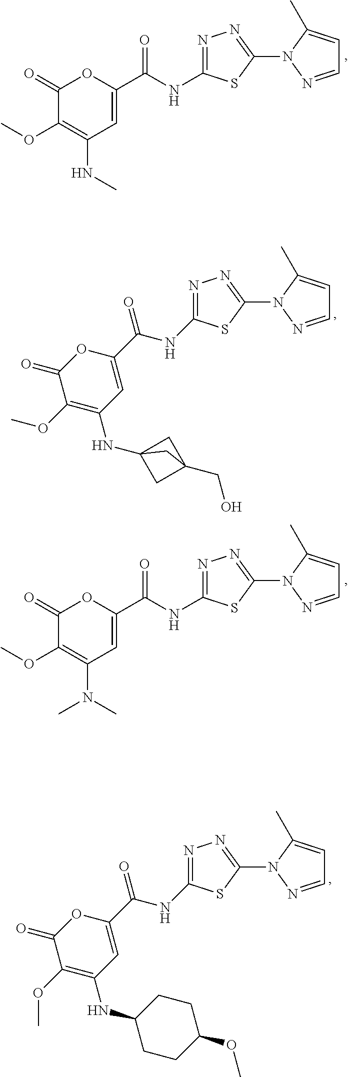 [SMILES: CNc1cc(C(=O)Nc2nnc(-n3nccc3C)s2)oc(=O)c1OC ; COc1c(NC23CC(CO)(C2)C3)cc(C(=O)Nc2nnc(-n3nccc3C)s2)oc1=O ; COc1c(N(C)C)cc(C(=O)Nc2nnc(-n3nccc3C)s2)oc1=O ; COc1c(N[C@H]2CC[C@@H](OC)CC2)cc(C(=O)Nc2nnc(-n3nccc3C)s2)oc1=O] 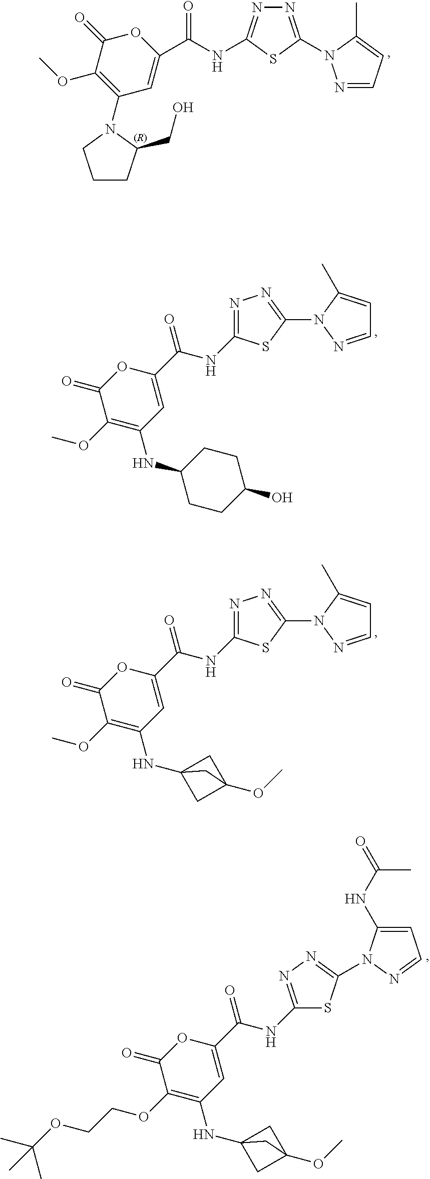 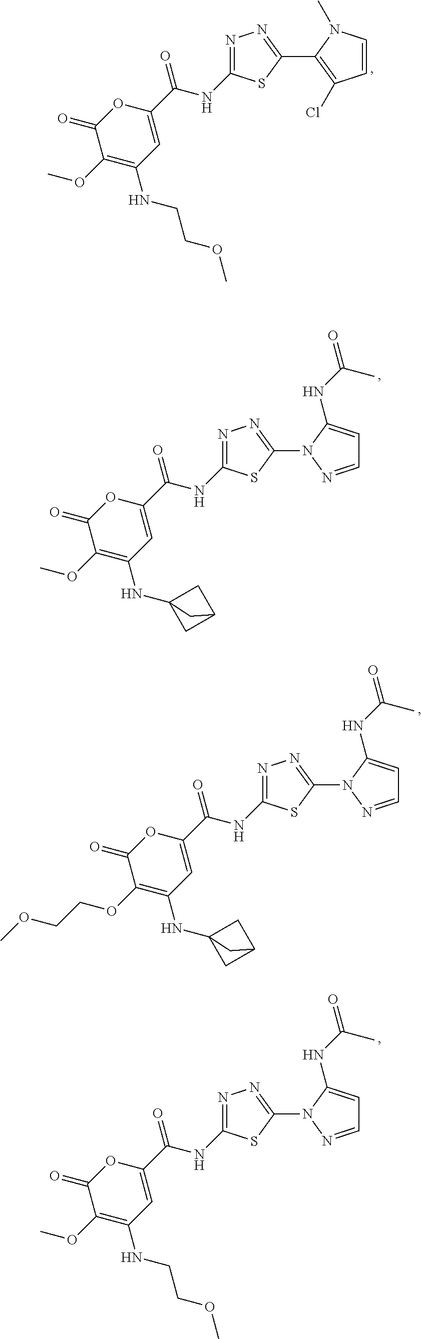 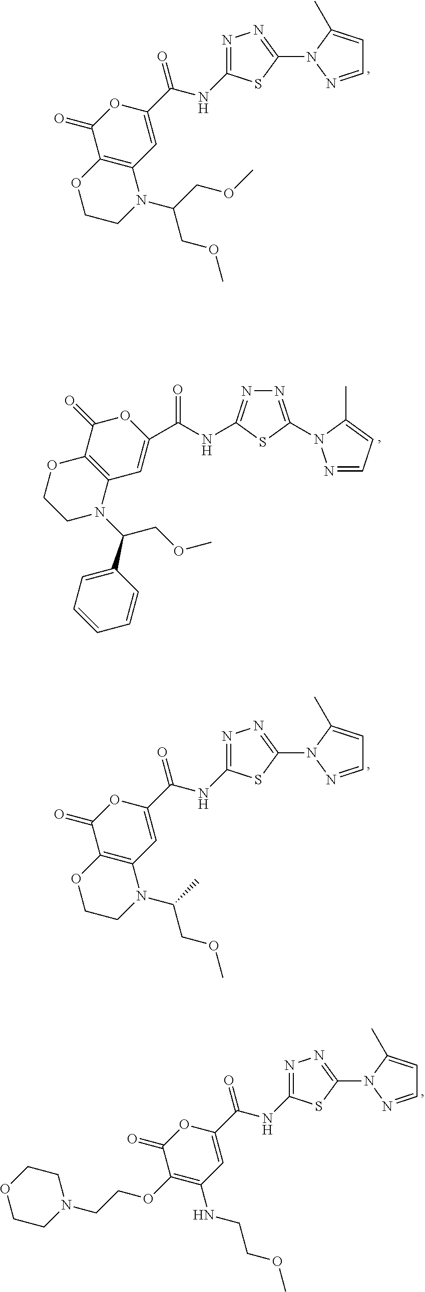 [SMILES: COCC(COC)N1CCOc2c1cc(C(=O)Nc1nnc(-n3nccc3C)s1)oc2=O ; COC[C@@H](c1ccccc1)N1CCOc2c1cc(C(=O)Nc1nnc(-n3nccc3C)s1)oc2=O ; COC[C@@H](C)N1CCOc2c1cc(C(=O)Nc1nnc(-n3nccc3C)s1)oc2=O ; COCCNc1cc(C(=O)Nc2nnc(-n3nccc3C)s2)oc(=O)c1OCCN1CCOCC1] 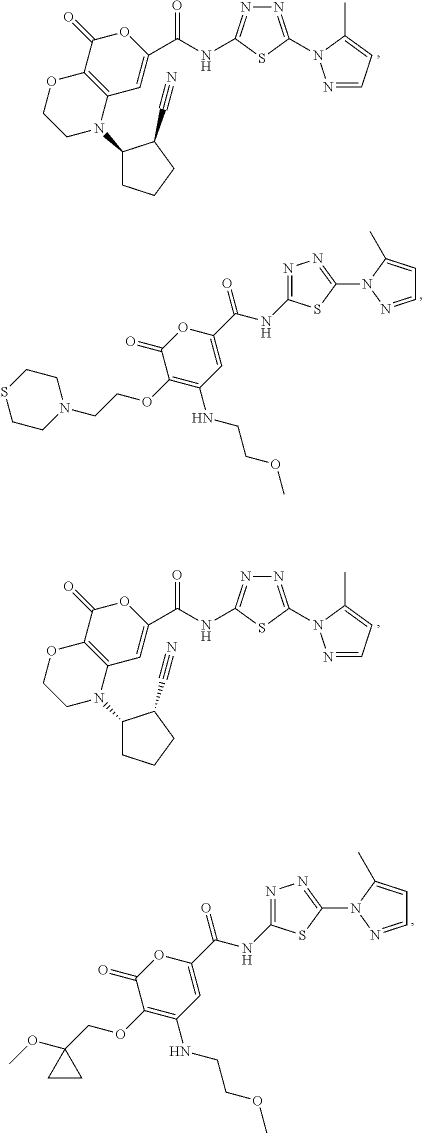 [SMILES: Cc1ccnn1-c1nnc(NC(=O)c2cc3c(c(=O)o2)OCCN3[C@@H]2CCC[C@@H]2C#N)s1 ; COCCNc1cc(C(=O)Nc2nnc(-n3nccc3C)s2)oc(=O)c1OCCN1CCSCC1 ; Cc1ccnn1-c1nnc(NC(=O)c2cc3c(c(=O)o2)OCCN3[C@H]2CCC[C@H]2C#N)s1 ; COCCNc1cc(C(=O)Nc2nnc(-n3nccc3C)s2)oc(=O)c1OCC1(OC)CC1] 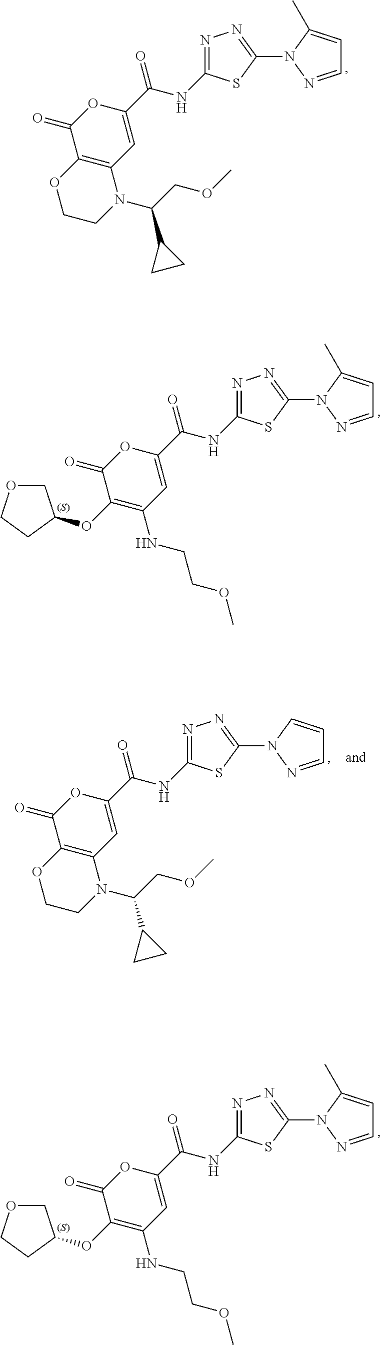
or a pharmaceutically acceptable salt of any of the foregoing.

33. (canceled)

34. A method of preparing a compound of Formula (I): 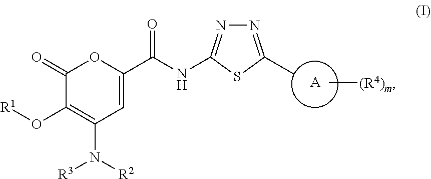
or a salt thereof, wherein Ring A, R¹, R², R³, R⁴, and m are defined in claim 1, the method comprising peptide coupling of a compound of Formula (H-1), or salt thereof, with a compound of Formula (N), or salt thereof: 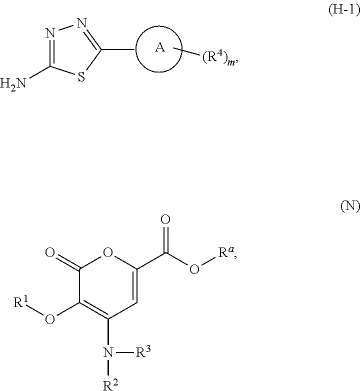
wherein R^{a} is hydrogen, C₁₋₆ alkyl, or C₁₋₆ haloalkyl, to provide a compound of Formula (I), or salt thereof.

35-36. (canceled)

37. A method of preparing a compound of Formula (I): 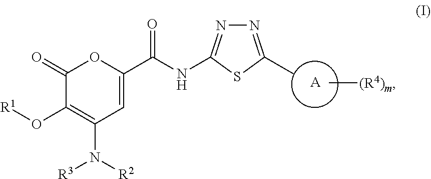
or a salt thereof, wherein Ring A, R¹, R², R³, R⁴, and m are defined in claim 1, the method comprising cross-coupling of the amine of Formula (K), or salt thereof, with a compound of Formula (J-1), or salt thereof: 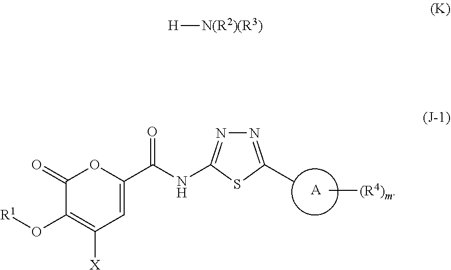
wherein X is Cl, Br, or I, to provide a compound of Formula (I), or salt thereof.

38-43. (canceled)

44. A method of preparing a compound of Formula (I-BC-a): 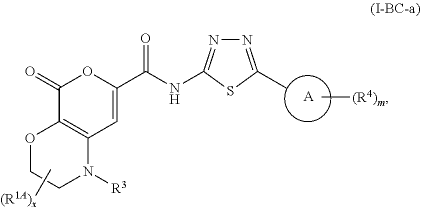
wherein Ring A, R¹, R³, R⁴, and m are defined in claim 1, the method comprising cyclizing a compound of Formula (P-1): 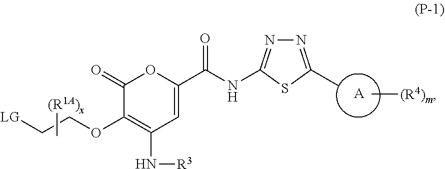
or salt thereof,
wherein LG is a leaving group.

45-46. (canceled)

47. A pharmaceutical composition comprising the compound of claim 1, or a pharmaceutically acceptable salt thereof, and a pharmaceutically acceptable carrier.

48. A method of treating a disease or disorder in a subject in need thereof, comprising administering to the subject a compound of claim 1, or a pharmaceutically acceptable salt thereof, or a pharmaceutical composition thereof.

49. (canceled)

50. A method of modulating cGAS activity, comprising contacting a cell with a compound of claim 1, or a pharmaceutically acceptable salt thereof, or a pharmaceutical composition thereof.

51. The compound of claim 1, or a pharmaceutically acceptable salt thereof, wherein R¹ is: [chemical structure]
